(19) United States
(12) Patent Application Publication
(10) Pub. No.: US 2024/0246937 A1
(43) Pub. Date: Jul. 25, 2024

(54) IL4I1 INHIBITORS AND METHODS OF USE

(71) Applicants: George Madalin GIAMBASU, Boston, MA (US); Andrew M. HAIDLE, Somerville, MA (US); Brett A. HOPKINS, Brownsburg, IN (US); James P. JEWELL, Boston, MA (US); Matthew A. LARSEN, Templeton, CA (US); Charles A. LESBURG, Waban, MA (US); Ping LIU, Boston, MA (US); Qinglin PU, Boston, MA (US); Sulagna SANYAL, Belmont, MA (US); Phieng SILIPHAIVANH, Boston, MA (US); Matthew TUDOR, Philadelphia, PA (US); Catherine M. WHITE, Boston, MA (US); Xin YAN, Boston, MA (US); Lianyun ZHAO, Shanghai (CN); Xiao Mei ZHENG, Boston, MA (US); William P. KAPLAN, Boston, MA (US); Michaelyn Claire LUX, Boston, MA (US); Derun LI, West Roxbury, MA (US); Rebecca JOHNSON, South San Francisco, CA (US); MERCK SHARP & DOHME LLC, Rahway, NJ (US); MSD R&D (CHINA) CO., LTD., Beijing (CN)

(72) Inventors: George Madalin GIAMBASU, Natick, MA (US); Andrew M. HAIDLE, Somerville, MA (US); Brett A. HOPKINS, Brownsburg, IN (US); James P. JEWELL, Newton, MA (US); Matthew A. LARSEN, Templeton, CA (US); Charles A. LESBURG, Waban, MA (US); Ping LIU, Westfield, NJ (US); Qinglin PU, Needham, MA (US); Sulagna SANYAL, Belmont, MA (US); Phieng SILIPHAIVANH, Newton, MA (US); Matthew TUDOR, Philadelphia, PA (US); Catherine M. WHITE, Canton, MA (US); Xin YAN, Newton Highlands, MA (US); Lianyun ZHAO, Shanghai (CN); Xiao Mei ZHENG, Dover, MA (US); William P. KAPLAN, Moseley, VA (US); Michaelyn Claire LUX, Brookline, MA (US); Derun LI, West Roxbury, MA (US); Rebecca JOHNSON, Oakland, CA (US)

(73) Assignees: MERCK SHARP & DOHME LLC, Rahway, NJ (US); MSD R&D (CHINA) CO., LTD., Beijing (CN)

(21) Appl. No.: 18/556,196

(22) PCT Filed: Apr. 28, 2022
(86) PCT No.: PCT/US2022/026626

§ 371 Date: Oct. 19, 2023

(30) Foreign Application Priority Data
Apr. 30, 2021 (WO) PCT/CN2021/091534

(51) Int. Cl. C07D 403/06; C07D 401/06; C07D 401/14; C07D 403/10; C07D 413/06; C07D 413/14; C07D 417/06; C07D 417/14; C07D 487/04

(52) U.S. Cl. CPC C07D 403/06; C07D 401/06; C07D 401/14; C07D 403/10; C07D 413/06; C07D 413/14; C07D 417/06; C07D 417/14; C07D 487/04

(57) ABSTRACT

The invention discloses a synthesizing method of benzimidazole derivant and intermediate, which is characterized by the following: proceeding arylation among aryl halide and primary amine; adopting L-pro as additive, cuprous iodide as catalyst; proceeding molecular arylation coupling reaction for aryl iodide or aryl bromide and primary amine; adopting iodo phenylamine compound and bromophenylamine compound as starting material; synthesizing the material through several-step transformation.

### FIELD OF THE INVENTION

The present invention is directed to IL4I1 inhibitors. Specifically, the IL4I1 inhibitors described herein can be useful in preventing, treating or acting as a remedial agent for IL4I1-related diseases.

### BACKGROUND OF THE INVENTION

IL4I1 is a glycosylated protein that belongs to the L-amino-acid oxidase (LAAO) family of flavin adenine dinucleotide (FAD)-bound enzymes. IL4I1 is secreted from certain cells and performs oxidative deamination of phenylalanine into phenylpyruvate, liberating H₂O₂ and NH₃.

The highest production of IL4I1 is found in cells of myeloid origin (monocyte/macrophages and dendritic cells) of the human immune system, particularly after stimulation with inflammatory and T helper type 1 (Th1) stimuli. Accordingly, IL4I1 is strongly produced by dendritic cell and macrophage populations from chronic Th1 granulomas of sarcoidosis and tuberculosis, but not Th2 granulomas (schistosomiasis). Moreover, tumor-infiltrating macrophages from various histological types of tumors strongly produce IL4I1. Molinier-Frenkel V., Prevost-Blondel A. and Castellano F., The IL4I1 Enzyme: A New Player in the Immunosuppressive Tumor Microenvironment, Cells, 2019, 8, 757-765.

The presence of IL4I1-producing cells in the tumor cell microenvironment restrains the anti-tumor immune response by directly limiting the proliferation and functionality of cytotoxic T cells and Th1 cells, or indirectly by facilitating the accumulation of Treg cells. Analyses of human tumor and normal tissue biopsies have identified increased expression of both IL4I1 mRNA and protein in tumor infiltrating myeloid cells. The Cancer Genome Atlas (TCGA) indicates that, among solid tumors, endometrial carcinoma contains the highest levels of IL4I1 mRNA expression, followed by serious ovarian and triple negative breast cancers. Phenylpyruvic acid, the product of phenylalanine oxidation by IL4I1, is elevated in endometrial and ovarian tumor samples relative to matched adjacent tissue from the same patients. Furthermore, accumulation of detectable phenylpyruvic acid in the tumor samples is dependent on the presence of IL4I1 itself.

Currently there are no specific inhibitors available against IL4I1. Some molecules have been shown to inhibit the related LAAOs found in snake venom, but they are generally non-selective and have little activity. Therefore, there is a need for specific inhibitors of IL4I1. More specifically there is a need for compounds that specifically inhibit IL4I1 and can be useful for the treatment of indications where IL4I1 is most expressed and/or active, including endometrial, ovarian and triple negative breast cancers.

### BRIEF SUMMARY OF THE INVENTION

### Compounds

Described herein are compounds of Formula I:

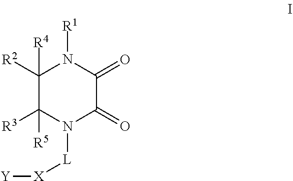

and pharmaceutically acceptable salts thereof, wherein are R¹, R², R³, R⁴, R⁵ L, X and Y are described below.

The compounds described herein are IL4I1 inhibitors, which can be useful in the prevention, treatment or amelioration of IL4I1-related diseases.

Also described herein are methods of preventing, treating or ameliorating the symptoms of cancer comprising administering to a patient in need thereof a compound described herein, or a pharmaceutically acceptable salt thereof.

Also described are uses of a compound described herein, or a pharmaceutically acceptable salt thereof, to prevent, treat or ameliorate the conditions of cancer in a patient in need thereof.

Also described are pharmaceutical compositions comprising a compound described herein, or a pharmaceutically acceptable salt thereof, and a pharmaceutically acceptable carrier.

Also described are pharmaceutical compositions comprising a compound described herein and a pharmaceutically acceptable carrier.

Also described herein are methods of preventing, treating or ameliorating the symptoms of cancer comprising administering to a patient in need thereof a compound described herein, or a pharmaceutically acceptable salt thereof, and another therapeutic agent.

Also described are uses of a compound described herein, or a pharmaceutically acceptable salt thereof, in combination with another therapeutic agent to prevent, treat or ameliorate the conditions of cancer in a patient in need thereof.

Also described are pharmaceutical compositions comprising a compound described herein, or a pharmaceutically acceptable salt thereof, another therapeutic agent and a pharmaceutically acceptable carrier.

Also described herein are pharmaceutical compositions comprising a compound described herein, another therapeutic agent, and a pharmaceutically acceptable carrier.

### DETAILED DESCRIPTION OF THE INVENTION

Described herein are compounds having a structural, Formula I.

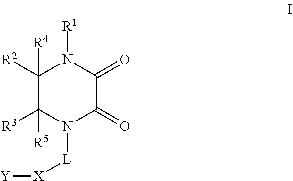

or a pharmaceutically acceptable salt thereof, wherein,
R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is unsubstituted or substituted with at least one substituent selected from the group consisting of oxo, —CN, —OH, halogen, C₁-C₆alkyl, C₁-C₆haloalkyl, phenyl, heteroaryl and C₃-C₆cycloalkyl and wherein the heteroaryl is unsubstituted or substituted with C₁-C₆alkoxy, or R¹, and the nitrogen to which R¹ is attached, is taken with R⁴ and forms a nitrogen-containing ring;
R² is H, C₁-C₆alkyl or together with R³ forms a bond;
R³ is H, C₁-C₆alkyl or together with R² forms a bond;
R⁴ is H, halogen, or C₁-C₆alkyl or R⁴ is taken with R¹, and the nitrogen to which R¹ is attached, and forms a nitrogen-containing ring, or R⁴ is taken with R⁵ and forms a C₃-C₆cycloalkyl;
R⁵ is H, halogen, or C₁-C₆alkyl, or R⁵ is taken with R⁴ and forms a C₃-C₆cycloalkyl;
L is a C₁-C₆alkylene or a C₃-C₆cycloalkylene linker;
X is phenyl, C₃-C₆cycloalkyl, or a nitrogen-containing ring, wherein the phenyl, C₃-C₆cycloalkyl or nitrogen-containing ring is unsubstituted or substituted with one or two substituents independently selected from the group consisting of halogen, —OH and oxo; and
Y is hydrogen, halogen, C₁-C₆alkoxy, phenyl, halogen-substituted phenyl, C¹-C⁶alkyl-substituted phenyl, nitrogen-containing ring, CN-substituted phenyl, a halogen-substituted nitrogen-containing ring, or a haloC₁-C₆alkyl-substituted nitrogen containing ring.

In certain embodiments, described herein are compounds of Formula I:

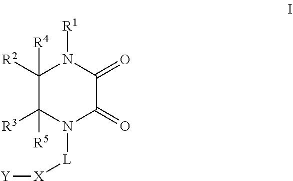

wherein, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is unsubstituted or substituted with one to three substituents independently selected independently from the group consisting of —CN, halogen, C₁-C₆alkyl, phenyl and C₃-C₆cycloalkyl;
R² is H, C₁-C₆alkyl or together with R³ forms a bond;
R³ is H, C₁-C₆alkyl or together with R² forms a bond;
R⁴ is H, halogen, or C₁-C₆alkyl;
R⁵ is H, halogen, or C₁-C₆alkyl;
L is a C₁-C₆alkylene or a C₃-C₆cycloalkylene linker;
X is phenyl, C₃-C₆cycloalkyl or a nitrogen-containing ring, wherein the phenyl, C₃-C₆cycloalkyl or nitrogen-containing ring is unsubstituted or substituted with at least one substituent selected from the group consisting of halogen and oxo; and
Y is hydrogen, halogen, C₁-C₆alkoxy, phenyl, halogen-substituted phenyl, nitrogen-containing ring, a halogen-substituted nitrogen-containing ring, or haloC₁-C₆alkyl-substituted nitrogen-containing ring.

With regards to the compounds described herein, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is unsubstituted or substituted with at least one substituent selected from the group consisting of oxo, —CN, —OH, halogen, C₁-C₆alkyl, C₁-C₆haloalkyl, phenyl, heteroaryl and C₃-C₆cycloalkyl, wherein the heteroaryl is unsubstituted or substituted with C₁-C₆alkoxy, or R¹, and the nitrogen to which R¹ is attached, is taken with R⁴ and forms a nitrogen-containing ring.

In certain embodiments, R¹ is C₁-C₆alkyl, C₃-C₆cycloalkyl, C₁-C₆alkylC₃-C₆cycloalkyl, aryl, heteroaryl, or heterocycloalkyl, wherein the C₁-C₆alkyl, C₃-C₆cycloalkyl, C₁-C₆alkylC₃-C₆cycloalkyl, aryl, heteroaryl or heterocycloalkyl is unsubstituted or substituted with at least one substituent selected from the group consisting of —CN, halogen, C₁-C₆alkyl, phenyl and C₃-C₆cycloalkyl.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is unsubstituted. In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is unsubstituted.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with at least one substituent selected from the group consisting of oxo, —CN, —OH, halogen, C₁-C₆alkyl, C₁-C₆haloalkyl, phenyl, heteroaryl and C₃-C₆cycloalkyl, wherein the heteroaryl is unsubstituted or substituted with C₁-C₆alkoxy.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with one to three substituents independently selected from the group consisting of oxo, —CN, —OH, halogen, C₁-C₆alkyl, C₁-C₆haloalkyl, phenyl, heteroaryl and C₃-C₆cycloalkyl, wherein the heteroaryl is unsubstituted or substituted with C₁-C₆alkoxy.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with one substituent selected from the group consisting of oxo, —CN, —OH, halogen, C₁-C₆alkyl, C₁-C₆haloalkyl, phenyl, heteroaryl and C₃-C₆cycloalkyl, wherein the heteroaryl is unsubstituted or substituted with C₁-C₆alkoxy.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with two substituents independently selected from the group consisting of oxo, —CN, —OH, halogen, C₁-C₆alkyl, C₁-C₆haloalkyl, phenyl, heteroaryl and C₃-C₆cycloalkyl, wherein the heteroaryl is unsubstituted or substituted with C₁-C₆alkoxy.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with three substituents independently selected from the group consisting of oxo, —CN, —OH, halogen, C₁-C₆alkyl, C₁-C₆haloalkyl, phenyl, heteroaryl and C₃-C₆cycloalkyl, wherein the heteroaryl is unsubstituted or substituted with C₁-C₆alkoxy.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with one to three substituents.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with one substituent.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with two substituents.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with three substituents.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with at least one substituent selected from the group consisting of —CN, halogen, C₁-C₆alkyl, phenyl and C₃-C₆cycloalkyl.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with a —CN. In certain embodiments, C₁-C₆alkyl, C₃-C₆cycloalkyl, C₁-C₆alkylC₃-C₆cycloalkyl, aryl, heteroaryl or heterocycloalkyl is substituted with a halogen. Suitable halogens include, but are not limited to, fluorine, chlorine, bromine or iodine.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with a C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with a phenyl.

In certain embodiments, R¹ is C₁-C₆alkyl, C₃-C₆cycloalkyl, C₁-C₆alkylC₃-C₆cycloalkyl, aryl, heteroaryl or heterocycloalkyl, wherein the C₁-C₆alkyl, C₃-C₆cycloalkyl, C₁-C₆alkylC₃-C₆cycloalkyl, aryl, heteroaryl or heterocycloalkyl is substituted with a C₃-C₆cycloalkyl. Suitable cycloalkyls include, but are not limited to, cyclopropyl, cyclobutyl, cyclopentyl and cyclohexyl.

In certain embodiments, R¹ is unsubstituted. In certain embodiments, R¹ is substituted with —CN, fluorine, methyl, phenyl or cyclopropyl.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with an oxo group.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with —CN.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with a halogen. Suitable halogens include, but are not limited to, fluorine, chlorine, bromine or iodine.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl. In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with methyl.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with C₁-C₆haloalkyl. Suitable haloalkyls include, but are not limited to, fluoromethyl, difluoromethyl, trifluoromethyl, fluoroethyl, difluoroethyl and trifluoromethyl.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with phenyl.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with a heteroaryl ring. In certain embodiments, the heteroaryl ring is a nitrogen-containing ring. In certain embodiments, the heteroaryl ring is pyridinyl. In certain embodiments, the heteroaryl is unsubstituted or substituted with C₁-C₆alkoxy.

In certain embodiments, R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is substituted with C₃-C₆cycloalkyl. Suitable cycloalkyls include, but are not limited to, cyclopropyl, cyclobutyl, cyclopentyl and cyclohexyl.

In certain embodiments, R¹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl.

In certain embodiments, R¹ is isopropyl, tert-butyl or neopentyl. In certain embodiments, R¹ is isopropyl or tert-butyl. In certain embodiments, R¹ is isopropyl, tert-butyl or neopentyl, wherein the isopropyl, tert-butyl or neopentyl is unsubstituted or substituted with fluorine. In certain embodiments, R¹ is isopropyl or tert-butyl, wherein the isopropyl or tert-butyl is unsubstituted or substituted with fluorine. In certain embodiments, R¹ is isopropyl. In certain embodiments, R¹ is tert-butyl. In certain embodiments, R¹ is neopentyl. In certain embodiments, R¹ is unsubstituted or substituted with fluorine.

In certain embodiments, R¹ is tert-butyl. In certain embodiments, R¹ is tert-butyl, substituted with heterocycle. In certain embodiments, R¹ is tert-butyl, substituted with pyridyl. In certain embodiments, R¹ is

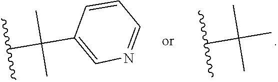

In certain embodiments, R¹ is C₃-C₆cycloalkyl. Suitable cycloalkyls include, but are not limited to, cyclopropyl, cyclobutyl, cyclopentyl, bicyclo[1.1.1]pentan-1-yl, bicyclo[3.1.0]hexane and cyclohexyl. In certain embodiments, R¹ is selected from the group consisting of.

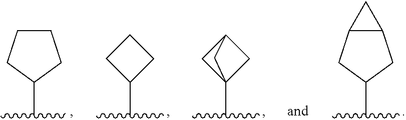

In certain embodiments, R¹ is selected from the group consisting of.

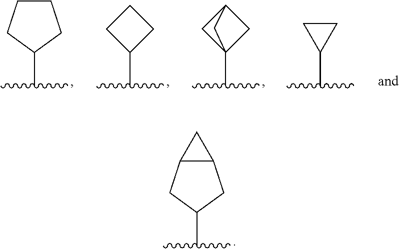

In certain embodiments, R¹ is

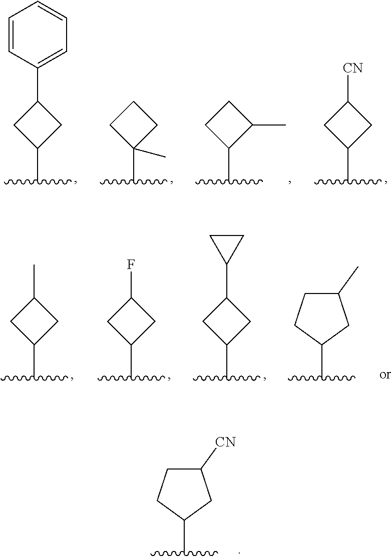

In certain embodiments, R¹ is C₁-C₆alkylC₃-C₆cycloalkyl. Suitable C₁-C₆alkylC₃-C₆cycloalkyl include, but are not limited to,

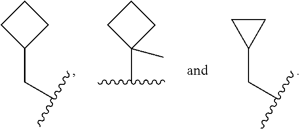

In certain embodiments, R¹ is aryl. Suitable aryls include, but are not limited to, phenyl and naphthyl. In certain embodiments, R¹ is phenyl.

In certain embodiments, R¹ is heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl.

In certain embodiments, R¹ is heterocycloalkyl. Suitable heterocycloalkyls include, but are not limited to, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and tetrahydropyran. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. In particular embodiments, R¹ is tetrahydrofuranyl.

In certain embodiments, R¹ is

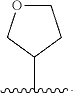 [SMILES: *C1CCOC1]

In certain embodiments, R¹ is

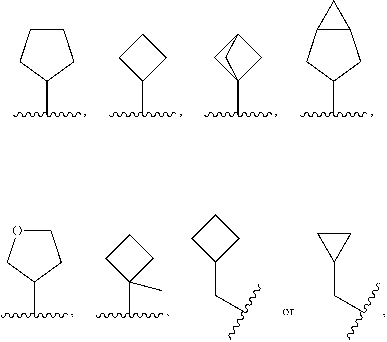

wherein R¹ is unsubstituted or substituted with —CN, fluorine, methyl, phenyl or cyclopropyl.

In certain embodiments, R¹ is C₁-C₆alkylOC₁-C₆alkyl. Suitable C₁-C₆alkylOC₁-C₆alkyls include, but are not limited to,

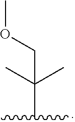 [SMILES: *C(C)(C)COC]

In certain embodiments, R¹ is

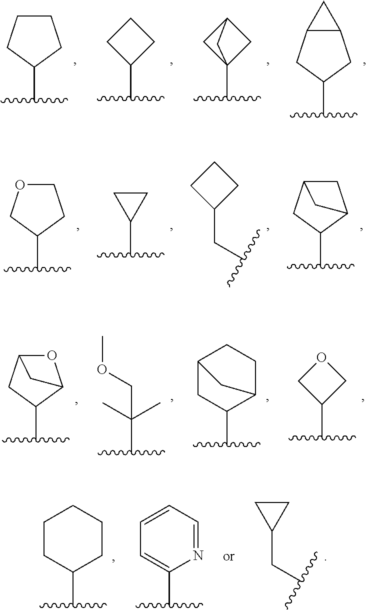

In certain embodiments, R¹ is

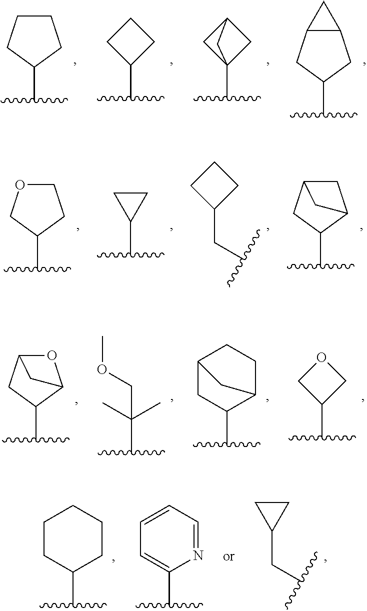

wherein R¹ is unsubstituted or substituted with one or two substituents independently selected from the group consisting of oxo, —CN, —OH, fluorine, methyl, phenyl, fluoromethyl, trifluoromethyl, pyridinyl, methoxypyridinyl and cyclopropyl.

In certain embodiments, R¹ and the nitrogen to which R¹ is attached is taken with R⁴ and forms a nitrogen-containing ring. In certain embodiments, R¹ and the nitrogen to which R¹ is attached is taken with R⁴ and forms a nitrogen-containing ring as shown below:

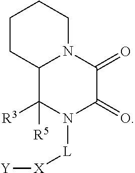

With regard to the compounds described herein, R² is H, C₁-C₆alkyl or together with R³ forms a bond. In certain embodiments, R² is H.

In certain embodiments, R² is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl. In particular embodiments, R² is methyl.

In certain embodiments, R² together with R³ forms a bond.

With regard to the compounds described herein, R³ is H, C₁-C₆alkyl or together with R² forms a bond. In certain embodiments, R³ is H.

In certain embodiments, R³ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl. In particular embodiments, R³ is methyl.

In certain embodiments, R³ together with R² forms a bond.

In certain embodiments, R² and R³ are both hydrogen. In certain embodiments, R² is methyl. In certain embodiments, R² is methyl and R³ is hydrogen. In certain embodiments, R³ is methyl. In certain embodiments, R³ is methyl and R² is hydrogen. In other embodiments, R² and R³ form a bond as shown in Formula II:

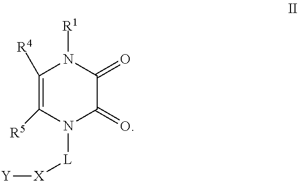

Concerning the compounds described herein, R⁴ is H, halogen, or C₁-C₆alkyl. In certain embodiments, R⁴ is H. In certain embodiments, R⁴ is halogen. Suitable halogens include, but are not limited to, fluorine, bromine, iodine and chlorine. In certain embodiments, R⁴ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl.

In certain embodiments, R⁴ is taken with R¹ and the nitrogen to which R¹ is attached and forms a nitrogen-containing ring. In certain embodiments, R⁴ is taken with R¹ and the nitrogen to which R¹ is attached and forms a nitrogen-containing ring as shown below:

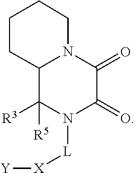

In certain embodiments, R⁴ is taken with R⁵ and forms a C₃-C₆cycloalkyl. Suitable cycloalkyls include, but are not limited to, cyclopropyl, cyclobutyl, cyclopentyl and cyclohexyl. In certain embodiments, R⁴ is taken with R⁵ and forms a C₃-C₆cycloalkyl as shown below:

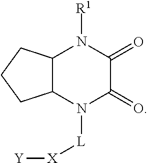

Concerning the compounds described herein, R⁵ is H, halogen, or C₁-C₆alkyl. In certain embodiments, R⁵ is H. In certain embodiments, R⁵ is halogen. Suitable halogens include, but are not limited to, fluorine, bromine, iodine and chlorine. In certain embodiments, R⁵ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl.

In certain embodiments, R⁴ and R⁵ are both hydrogen.

In certain embodiments, R², R³, R⁴ and R⁵ are all hydrogen.

In certain embodiments, R⁵ is taken with R⁴ and forms a C₃-C₆cycloalkyl. Suitable cycloalkyls include, but are not limited to, cyclopropyl, cyclobutyl, cyclopentyl and cyclohexyl. In certain embodiments, R⁴ is taken with R⁵ and forms a C₃-C₆cycloalkyl as shown below:

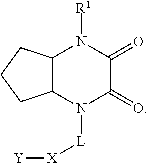

With regard to the compounds described herein, L is a C₁-C₆alkylene or C₃-C₆cycloalkylene linker. In certain embodiments, L is a C₁-C₆alkylene linker. Suitable alkylene linkers include, but are not limited to,

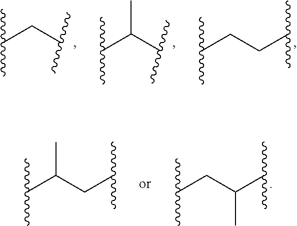

In certain embodiments, L is

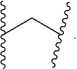 [SMILES: *C*]

In certain embodiments, L is

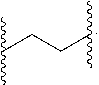 [SMILES: *CC*]

In certain embodiments, L is

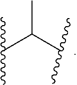 [SMILES: *C(*)C]

In certain embodiments, L is

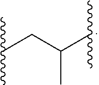 [SMILES: *CC(*)C]

In certain embodiments, L is a C₃-C₆cycloalkylene linker. Suitable cycloalkylene linkers include, but are not limited to

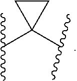 [SMILES: *C1(*)CC1]

With regard to the compounds described herein, X is phenyl, C₃-C₆cycloalkyl or a nitrogen-containing ring, wherein the phenyl, C₃-C₆cycloalkyl or nitrogen-containing ring is unsubstituted or substituted with at least one substituent selected from the group consisting of halogen, —OH and oxo.

In certain embodiments, X is phenyl, C₃-C₆cycloalkyl or a nitrogen-containing ring, wherein the phenyl, C₃-C₆cycloalkyl or nitrogen-containing ring is unsubstituted.

In certain embodiments, X is phenyl, C₃-C₆cycloalkyl or a nitrogen-containing ring, wherein the phenyl, C₃-C₆cycloalkyl or nitrogen-containing ring is substituted with a substituent with —OH.

In certain embodiments, X is phenyl, C₃-C₆cycloalkyl or a nitrogen-containing ring, wherein the phenyl, C₃-C₆cycloalkyl or nitrogen-containing ring is unsubstituted or substituted with at least one substituent selected from the group consisting of halogen and oxo.

In certain embodiments, X is phenyl. In certain embodiments, X is unsubstituted phenyl. In certain embodiments, X is phenyl, wherein the phenyl is substituted with halogen. In certain embodiments, X is phenyl, wherein the phenyl is substituted with fluorine. In other embodiments, X is 2-fluorophenyl.

In certain embodiments, X is C₃-C₆cycloalkyl. Suitable cycloalkyls include, but are not limited to, cyclopropyl, cyclobutyl, cyclopentyl, bicyclo[1.1.1]pentan-1-yl, bicyclo[3.1.0]hexane and cyclohexyl. In certain embodiments, X is

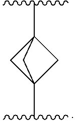 [SMILES: *C12CC(*)(C1)C2]

In certain embodiments, X is C₃-C₆cycloalkyl, wherein the C₃-C₆cycloalkyl is unsubstituted. In certain embodiments, X is C₃-C₆cycloalkyl, wherein the C₃-C₆cycloalkyl is substituted. In certain embodiments, X is C₃-C₆cycloalkyl, wherein the C₃-C₆cycloalkyl is substituted with one or two substituents independently selected from the group consisting of halogen, —OH and oxo. In certain embodiments, X is C₃-C₆cycloalkyl, wherein the C₃-C₆cycloalkyl is substituted with two fluorines. In certain embodiments, X is

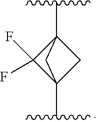 [SMILES: *C12CC(*)(C1)C2(F)F]

In certain embodiments, X is a nitrogen-containing ring. In certain embodiments, X is a monocyclic nitrogen-containing ring. In certain embodiments, X is a bicyclic nitrogen-containing ring. In certain embodiments, X is a nitrogen-containing heteroaryl. Suitable heteroaryls include,

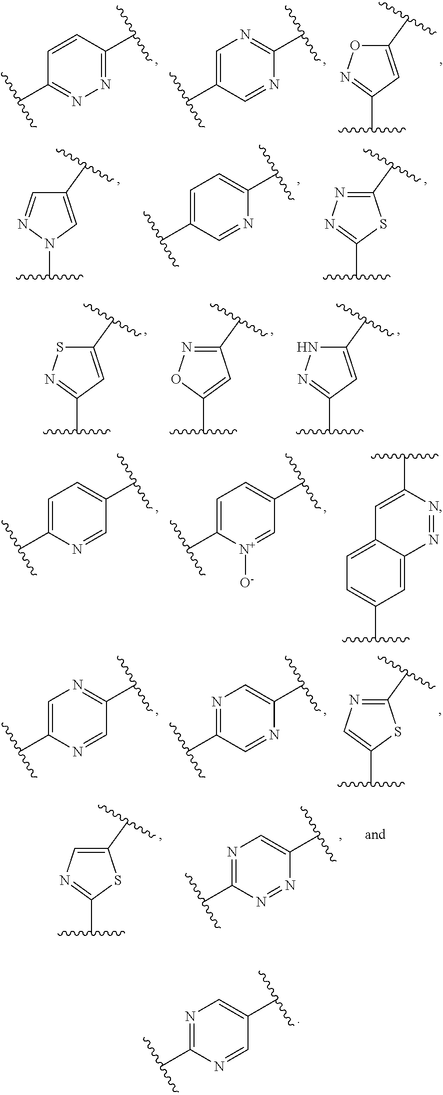

In certain embodiments, X is a bicyclic nitrogen-containing heteroaryl. In certain embodiments, X is a monocyclic nitrogen-containing heteroaryl.

In certain embodiments, X is a nitrogen-containing heterocycloalkyl. Suitable heterocycloalkyls include

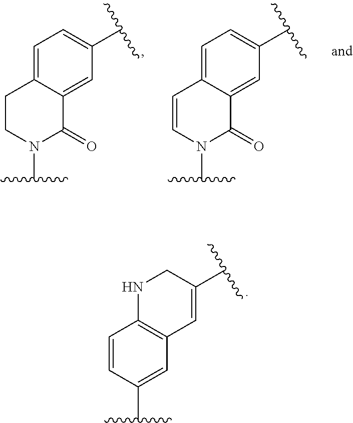 [SMILES: *c1ccc2c(c1)C(=O)N(*)CC2 ; *c1ccc2ccn(*)c(=O)c2c1 ; *C1=Cc2cc(*)ccc2NC1]

In certain embodiments, X is a nitrogen-containing heterocycloalkyl, wherein the nitrogen-containing heterocycloalkyl is substituted with an oxo.

In certain embodiments, X is a bicyclic nitrogen-containing heterocycloalkyl. In certain embodiments, X is a monocyclic nitrogen-containing heterocycloalkyl. In certain embodiments, X is a partially unsaturated nitrogen-containing ring. In certain embodiments, X is a bicyclic partially unsaturated nitrogen-containing ring. In certain embodiments, X is a monocyclic partially unsaturated nitrogen-containing ring.

In certain embodiments, X is an unsubstituted nitrogen-containing ring. In certain embodiments, X is a substituted nitrogen-containing ring. In certain embodiments, X is a nitrogen-containing ring, wherein the nitrogen-containing ring is substituted with halogen. In certain embodiments, X is a nitrogen-containing ring, wherein the nitrogen-containing ring is substituted with fluorine. In certain embodiments, X is a nitrogen-containing ring, wherein the nitrogen-containing ring is substituted with an oxo group.

In certain embodiments, X is pyrazolyl, thiazolyl, isoxazolyl, thiadiazolyl, pyridine-3-yl, 3-fluoro-pyridin-2-yl, pyridine-1-oxide, pyridazinyl, pyrazinyl, pyrimidinyl, triazinyl, 2-oxo-dihydroquinolinyl, 1-oxo-tetrahdyroisoquinolinyl, 1-oxo-dihydroisoquinolinyl, 2-cinnolinyl or 7-chlorocinnolinyl.

In certain embodiments, X is a nitrogen-containing ring, wherein the nitrogen containing ring is selected from the group consisting of

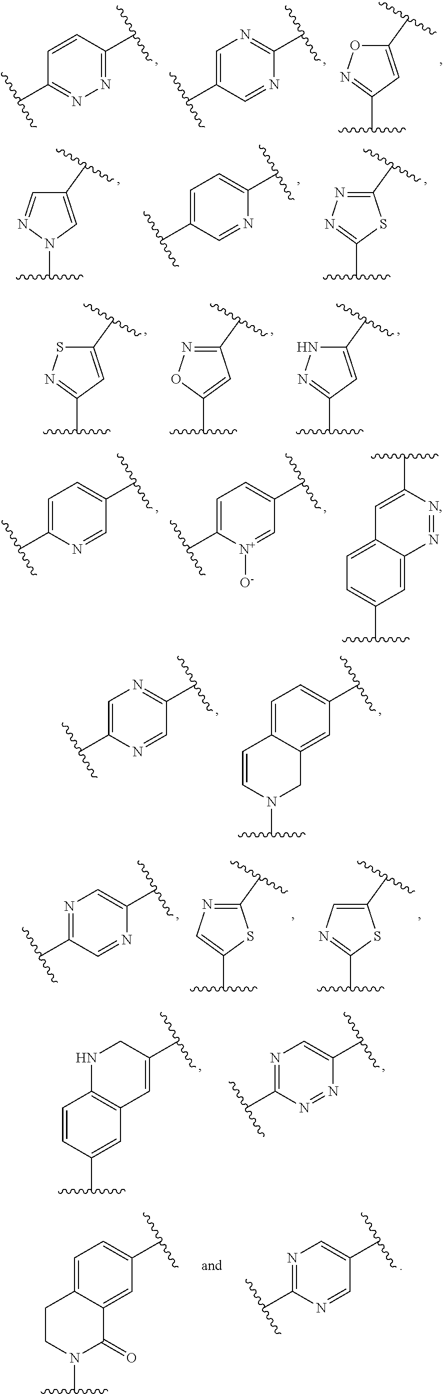

In certain embodiments, the nitrogen-containing ring is selected from the group consisting of

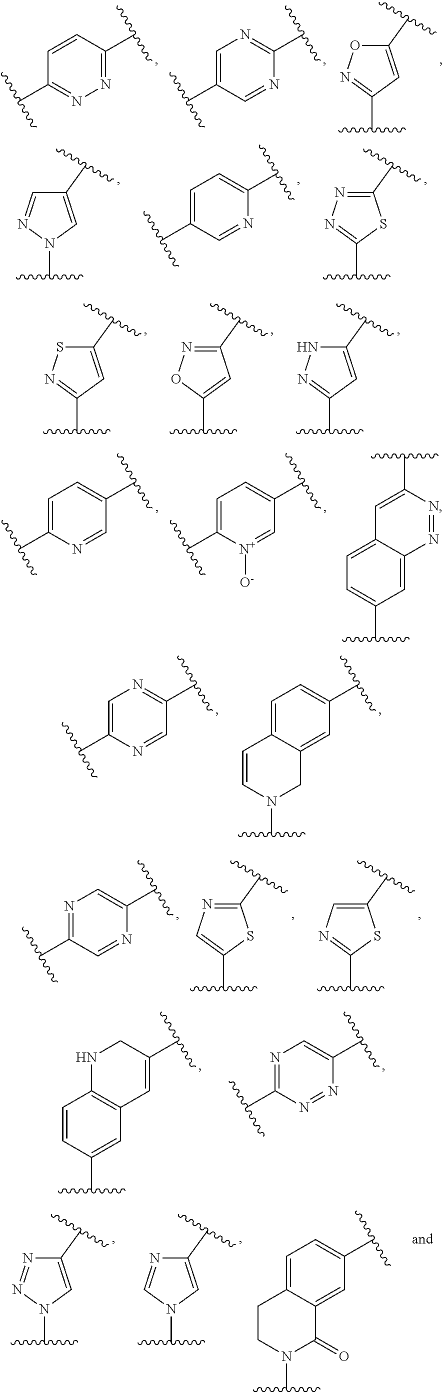

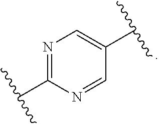 [SMILES: *c1cnc(*)nc1]

In certain embodiments, the nitrogen-containing ring is selected from the group consisting of

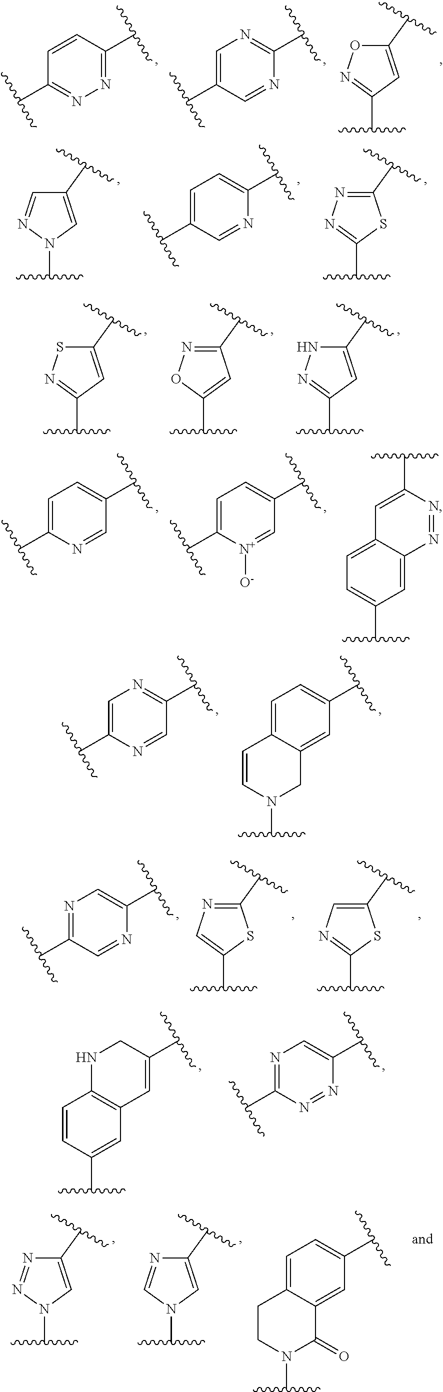

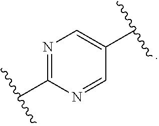

wherein X is unsubstituted or substituted with one or two substituents independently selected from the group consisting of fluorine and an oxo group.

In certain embodiments X is a nitrogen-containing ring, wherein the nitrogen containing ring

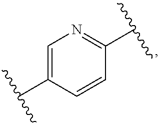

wherein X is substituted with fluorine.

In certain embodiments X is a nitrogen-containing ring, wherein the nitrogen containing ring is

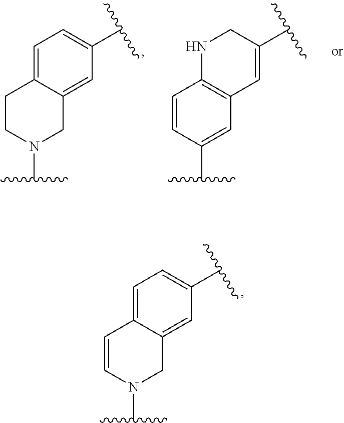

wherein X is substituted with an oxo group.

In certain embodiments X is

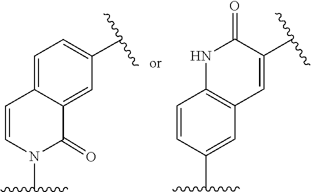 [SMILES: *c1ccc2ccn(*)c(=O)c2c1 ; *c1ccc2[nH]c(=O)c(*)cc2c1]

Such compounds can exist as, or

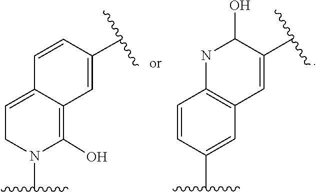 [SMILES: *c1ccc2c(c1)=C(O)N(*)CC=2 ; *C1=Cc2cc(*)ccc2NC1O]

With regard to the compounds described herein, Y is hydrogen, halogen, C₁-C₆alkoxy, phenyl, a halogen-substituted phenyl, C₁-C₆alkyl-substituted phenyl, a nitrogen-containing ring, —CN-substituted phenyl, a halogen-substituted nitrogen-containing ring, or a haloC₁-C₆alkyl-substituted nitrogen containing ring.

In certain embodiments, Y is hydrogen, halogen, C₁-C₆alkoxy, phenyl, a halogen-substituted phenyl, a nitrogen-containing ring, a halogen-substituted nitrogen-containing ring or a haloC₁-C₆alkyl-substituted nitrogen-containing ring.

In certain embodiments, Y is hydrogen. In certain embodiments, Y is halogen. Suitable halogens include, but are not limited to, chlorine, fluorine, bromine and iodine. In certain embodiments, Y is fluorine. In certain embodiments, Y is C₁-C₆alkoxy. Suitable alkoxys include, but are not limited to, methoxy, ethoxy, n-propoxy, isopropoxy and n-butoxy. In certain embodiments, Y is ethoxy or propoxy.

In certain embodiments, Y is phenyl. In certain embodiments, Y is a halogen-substituted phenyl. Suitable halogens include, but are not limited to, chlorine, fluorine, bromine and iodine. In certain embodiments, Y is phenyl substituted with fluorine. In certain embodiments, Y is phenyl substituted with one or two substituents independently selected from fluorine and bromine.

In certain embodiments, Y is a CN-substituted phenyl. In certain embodiments, Y is phenyl substituted with —CN.

In certain embodiments, Y is a C₁-C₆alkyl-substituted phenyl. In certain embodiments, Y is phenyl substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl. In certain embodiments, Y is phenyl substituted with methyl.

In certain embodiments, Y is a nitrogen-containing ring. In certain embodiments, Y is a halogen-substituted nitrogen-containing ring. In certain embodiments, Y is a haloC₁-C₆alkyl-substituted nitrogen-containing ring.

In certain embodiments, Y is phenyl, wherein the phenyl is substituted with one to two fluorines. In certain embodiments, Y is C₁-C₆alkoxy, wherein the C₁-C₆alkoxy is ethoxy, propoxy or butoxy. In certain embodiments, Y is C₁-C₆alkoxy, wherein the C₁-C₆alkoxy is propoxy.

In certain embodiments, Y is a monocyclic nitrogen-containing ring. Examples of a monocyclic nitrogen containing ring include

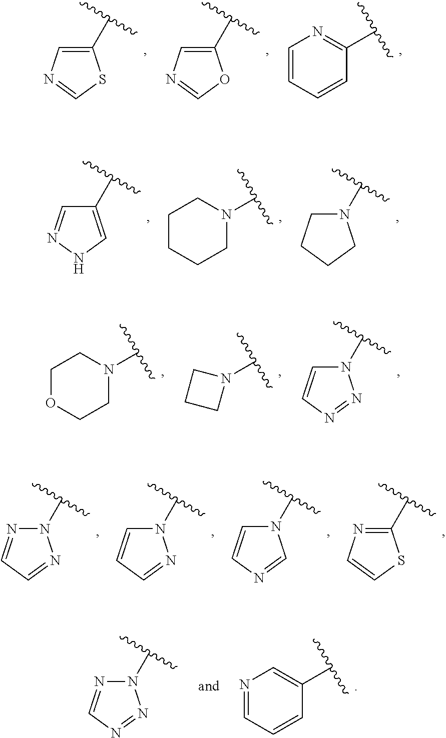

In certain embodiments, Y is a bicyclic nitrogen-containing ring. Examples of a bicyclic nitrogen-containing ring include

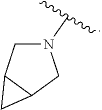 [SMILES: *N1CC2CC2C1]

In certain embodiments, Y is a nitrogen-containing heteroaryl. Examples of a nitrogen-containing heteroaryl include,

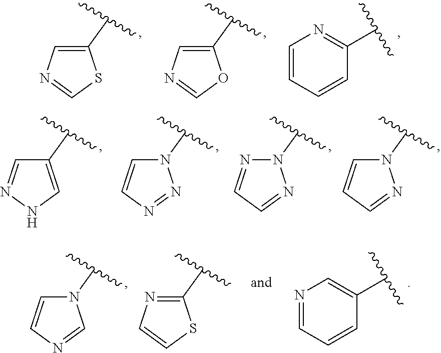

In certain embodiments, Y is nitrogen-containing heterocycloalkyl. Examples of suitable nitrogen-containing heterocycloalkyls include

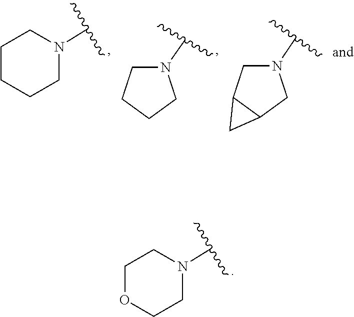 [SMILES: *N1CCCCC1 ; *N1CCCC1 ; *N1CC2CC2C1 ; *N1CCOCC1]

In certain embodiments, Y is a saturated nitrogen-containing ring. In certain embodiments, Y is a nitrogen-containing ring, wherein the nitrogen containing ring is selected from the group consisting of

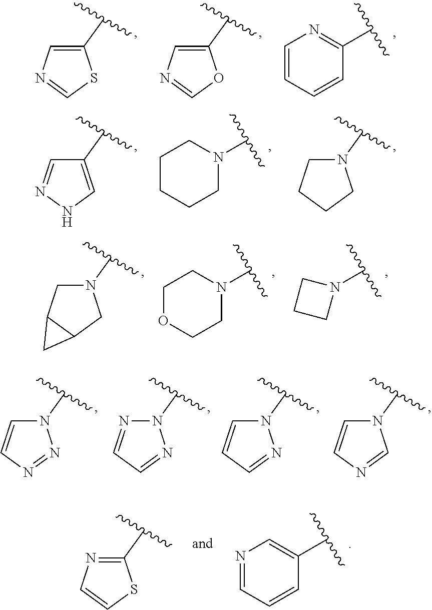

In certain embodiments, Y is azetidinyl, azabicyclo[3.1.0]hexanyl, pyrrolindinyl, piperidinyl, morpholino, imidazolyl, pyrazolyl, triazolyl, thiazolyl, oxazolyl or pyridine-3-yl.

In certain embodiments, Y is a halogen-substituted nitrogen-containing ring, wherein the halogen-substituted nitrogen containing ring is selected from the group consisting of

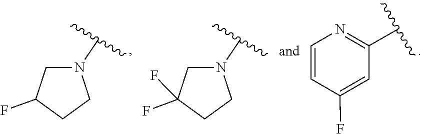 [SMILES: *N1CCC(F)C1 ; *N1CCC(F)(F)C1 ; *c1cc(F)ccn1]

In certain embodiments, Y is a haloC₁-C₆alkyl-substituted nitrogen-containing ring, wherein the haloC₁-C₆alkyl-substituted nitrogen-containing ring is

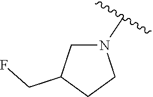 [SMILES: *N1CCC(CF)C1]

In certain embodiments, Y is hydrogen only when X is

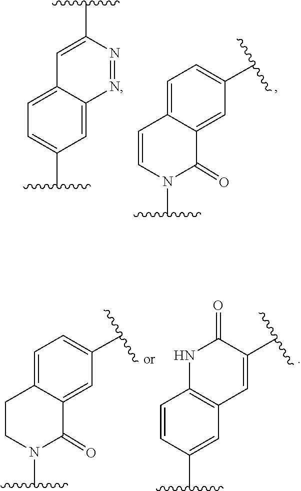 [SMILES: *c1ccc2cc(*)nnc2c1 ; *c1ccc2ccn(*)c(=O)c2c1 ; *c1ccc2c(c1)C(=O)N(*)CC2 ; *c1ccc2[nH]c(=O)c(*)cc2c1]

Also, described herein are the following compounds:

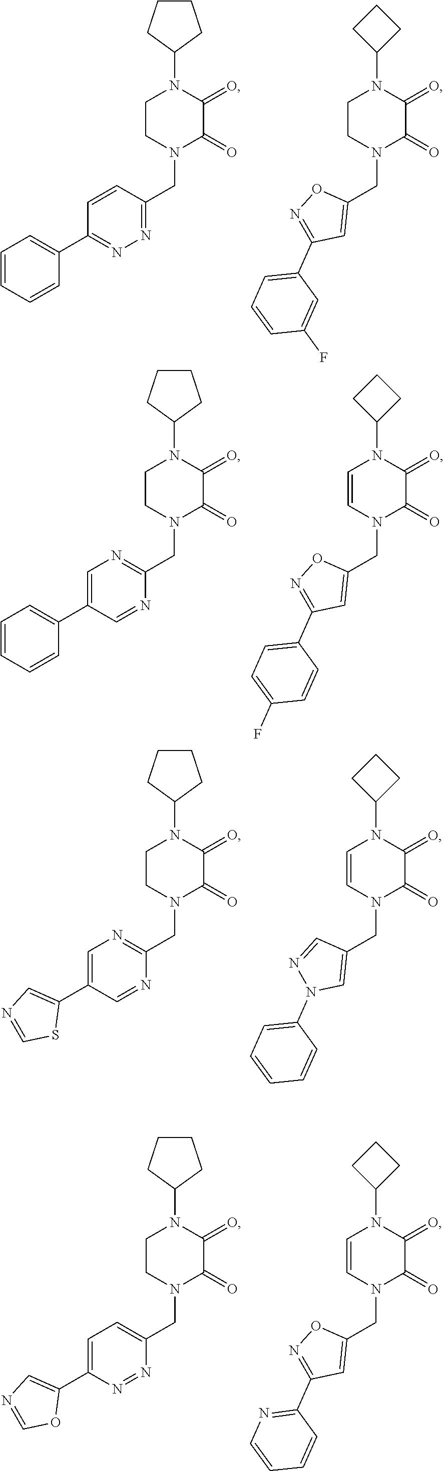

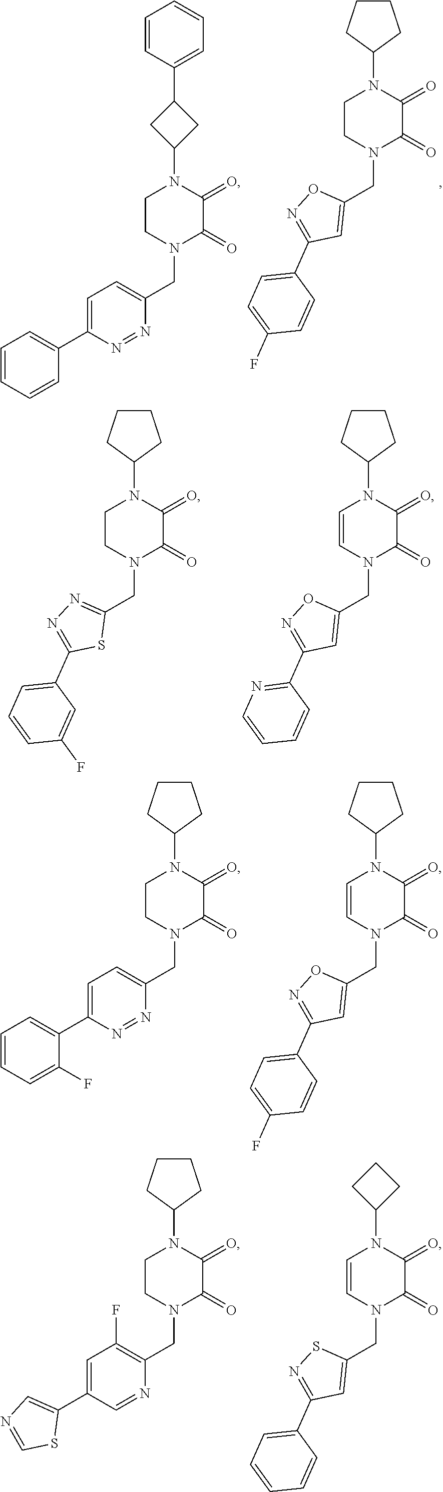

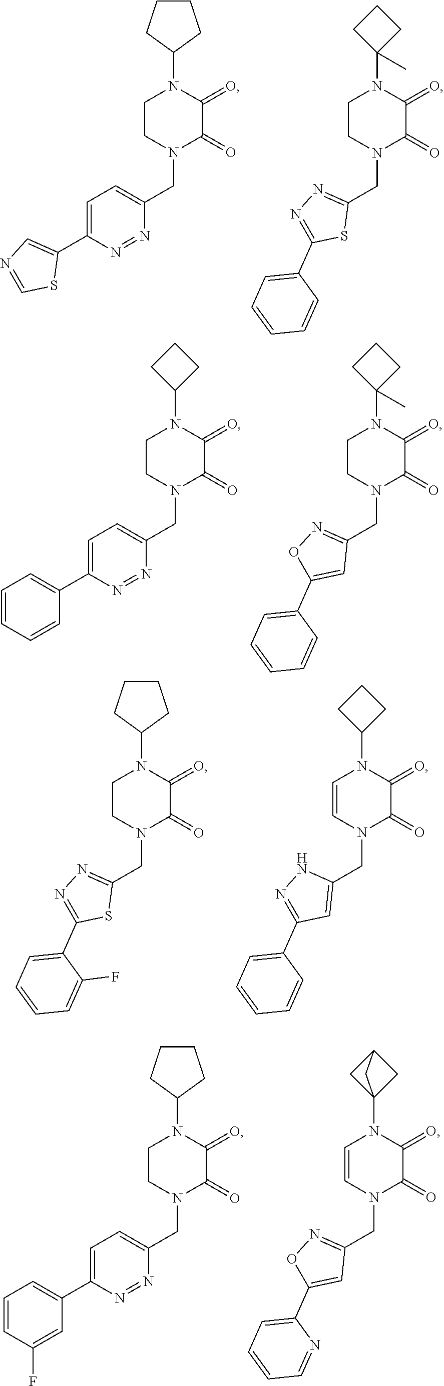

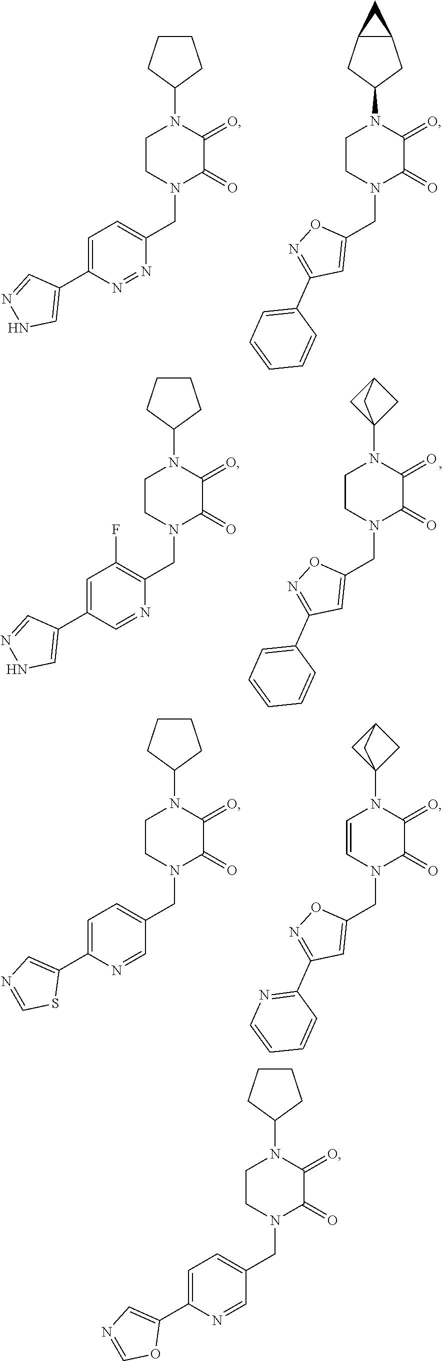

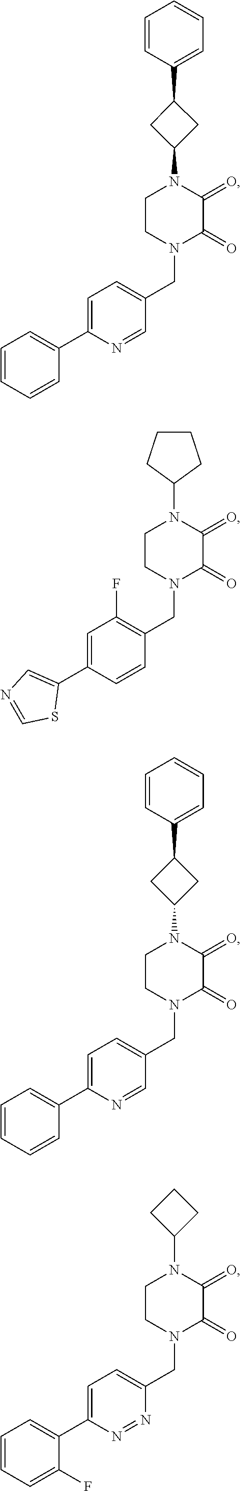 [SMILES: O=C1C(=O)N([C@H]2C[C@@H](c3ccccc3)C2)CCN1Cc1ccc(-c2ccccc2)nc1 ; O=C1C(=O)N(C2CCCC2)CCN1Cc1ccc(-c2cncs2)cc1F ; O=C1C(=O)N([C@H]2C[C@H](c3ccccc3)C2)CCN1Cc1ccc(-c2ccccc2)nc1 ; O=C1C(=O)N(C2CCC2)CCN1Cc1ccc(-c2ccccc2F)nn1]

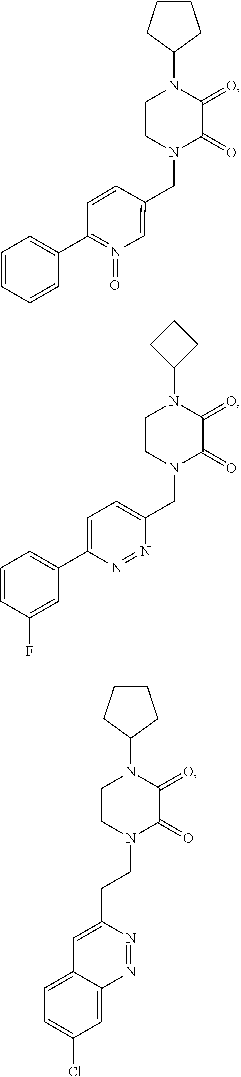 [SMILES: O=N/C=C(\C=C/Cc1ccccc1)CN1CCN(C2CCCC2)C(=O)C1=O ; O=C1C(=O)N(C2CCC2)CCN1Cc1ccc(-c2cccc(F)c2)nn1 ; O=C1C(=O)N(C2CCCC2)CCN1CCc1cc2ccc(Cl)cc2nn1]

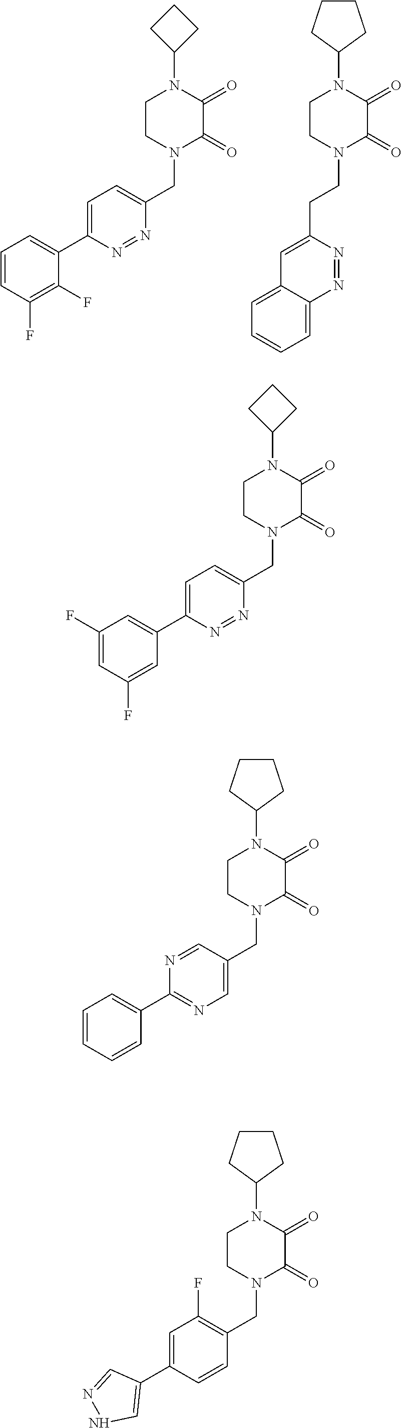

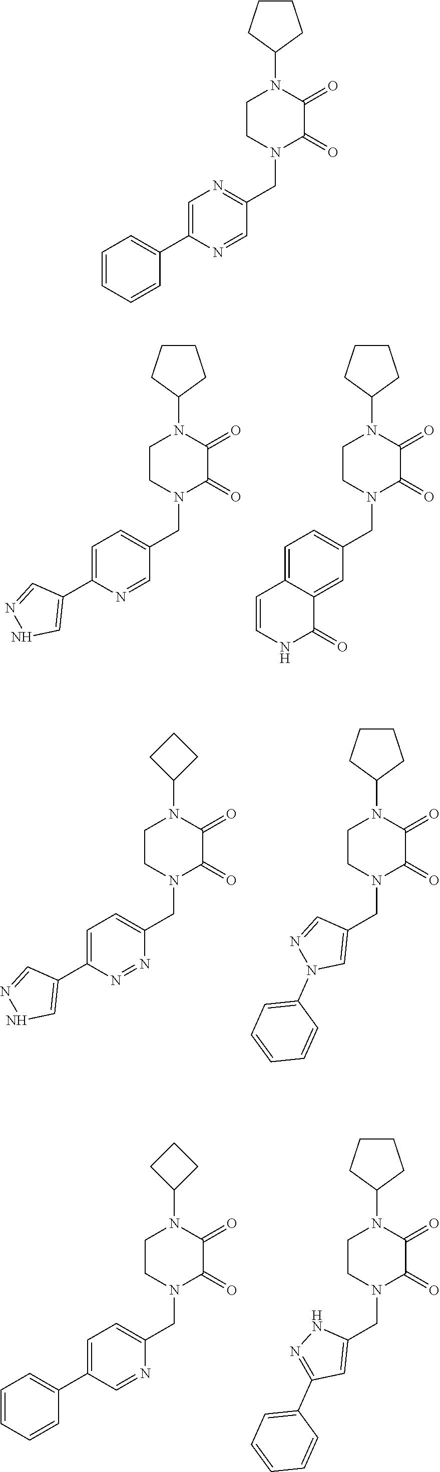

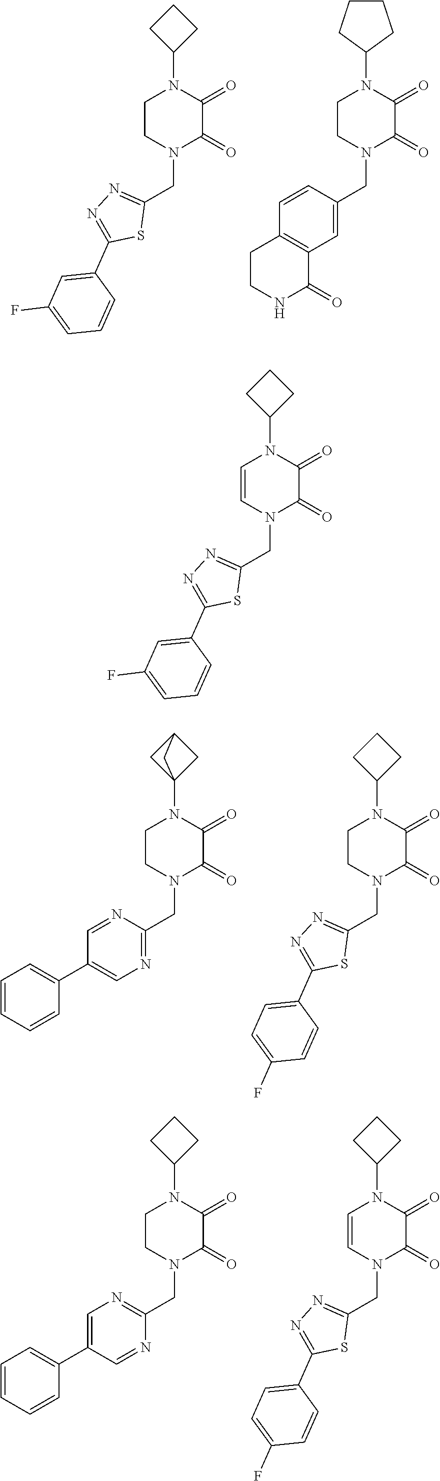

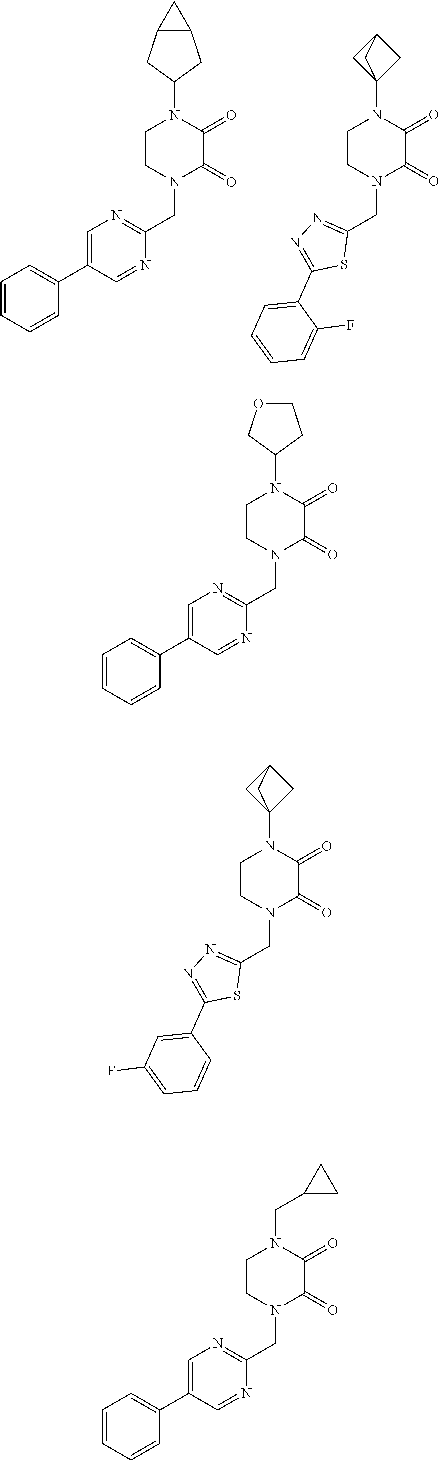

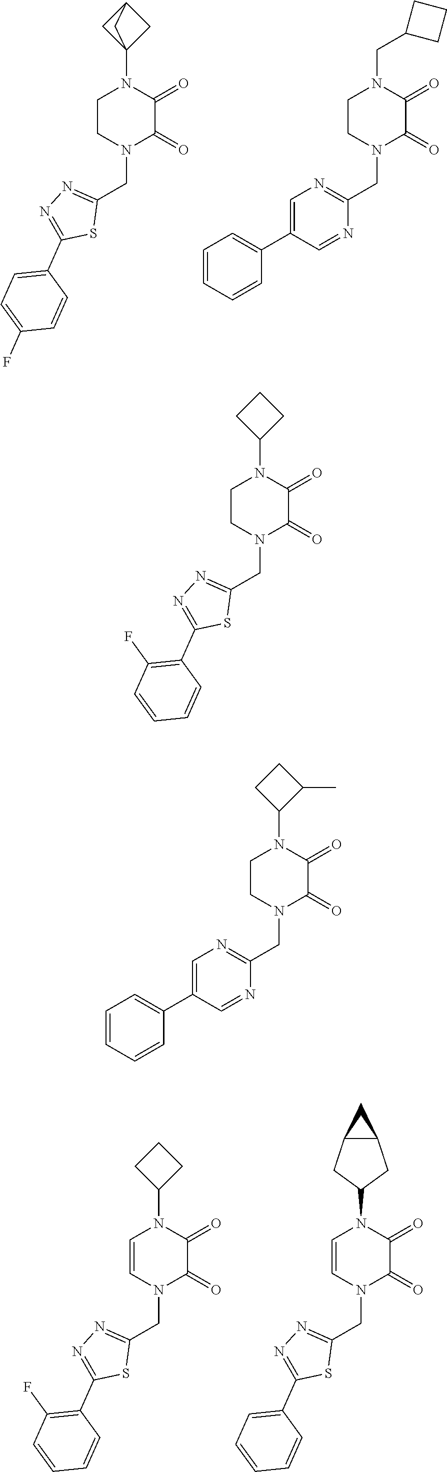

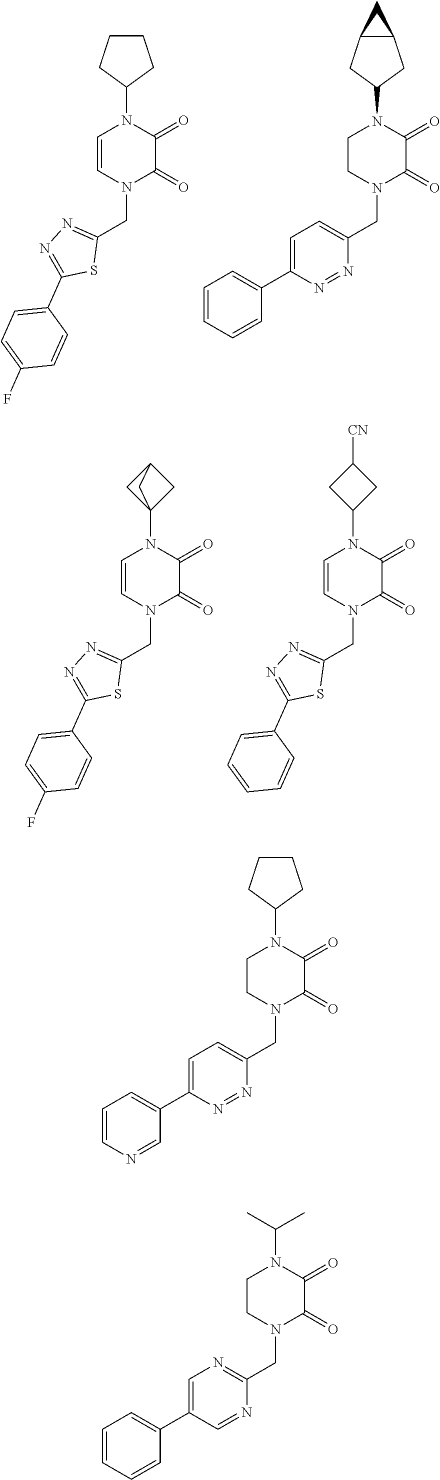

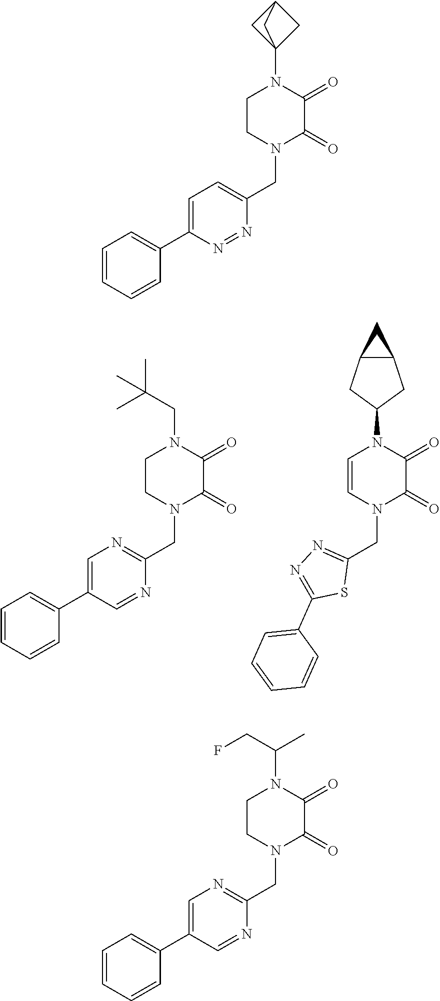 [SMILES: O=C1C(=O)N(C23CC(C2)C3)CCN1Cc1ccc(-c2ccccc2)nn1 ; CC(C)(C)CN1CCN(Cc2ncc(-c3ccccc3)cn2)C(=O)C1=O ; O=c1c(=O)n([C@H]2C[C@H]3C[C@H]3C2)ccn1Cc1nnc(-c2ccccc2)s1 ; CC(CF)N1CCN(Cc2ncc(-c3ccccc3)cn2)C(=O)C1=O]

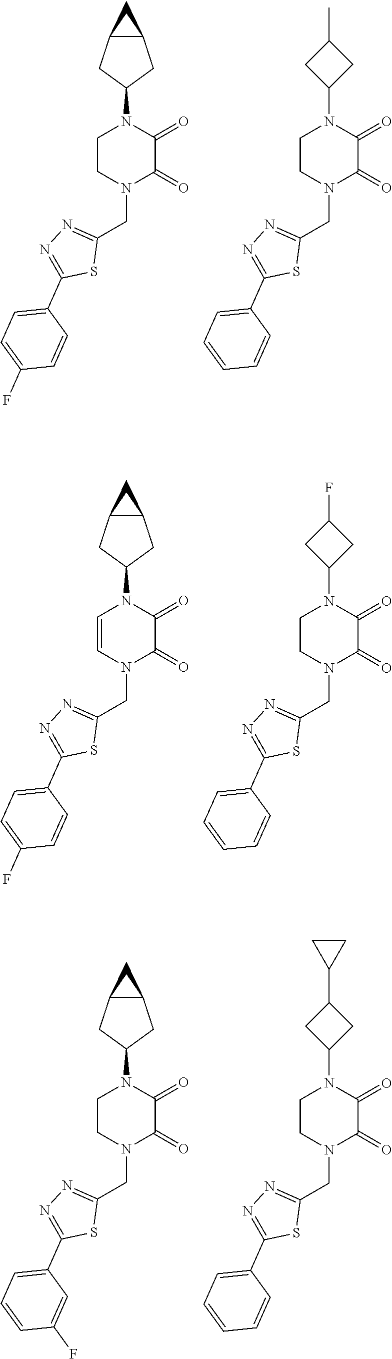 [SMILES: O=C1C(=O)N([C@H]2C[C@H]3C[C@H]3C2)CCN1Cc1nnc(-c2ccc(F)cc2)s1 ; CC1CC(N2CCN(Cc3nnc(-c4ccccc4)s3)C(=O)C2=O)C1 ; O=c1c(=O)n([C@H]2C[C@H]3C[C@H]3C2)ccn1Cc1nnc(-c2ccc(F)cc2)s1 ; O=C1C(=O)N(C2CC(F)C2)CCN1Cc1nnc(-c2ccccc2)s1 ; O=C1C(=O)N([C@H]2C[C@H]3C[C@H]3C2)CCN1Cc1nnc(-c2cccc(F)c2)s1 ; O=C1C(=O)N(C2CC(C3CC3)C2)CCN1Cc1nnc(-c2ccccc2)s1]

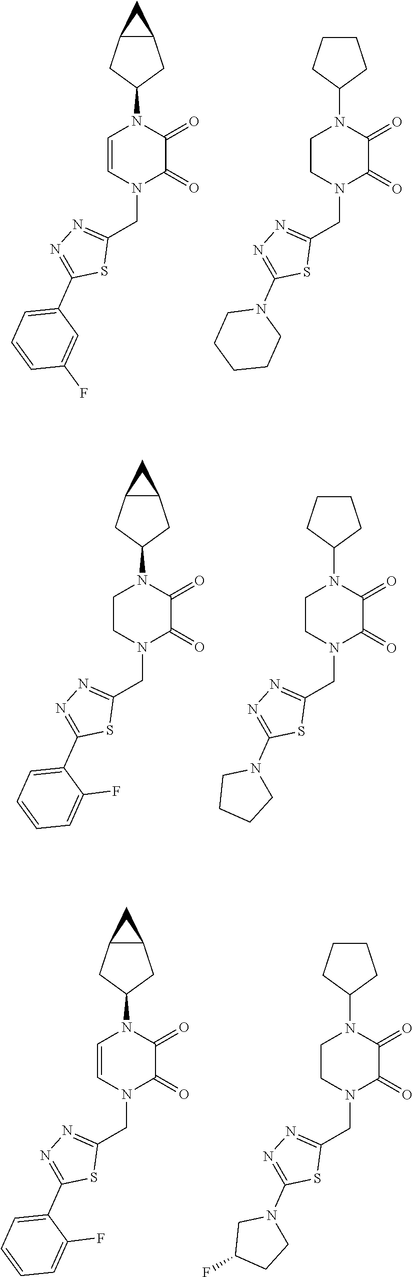 [SMILES: O=c1c(=O)n([C@H]2C[C@H]3C[C@H]3C2)ccn1Cc1nnc(-c2cccc(F)c2)s1 ; O=C1C(=O)N(C2CCCC2)CCN1Cc1nnc(N2CCCCC2)s1 ; O=C1C(=O)N([C@H]2C[C@H]3C[C@H]3C2)CCN1Cc1nnc(-c2ccccc2F)s1 ; O=C1C(=O)N(C2CCCC2)CCN1Cc1nnc(N2CCCC2)s1 ; O=c1c(=O)n([C@H]2C[C@H]3C[C@H]3C2)ccn1Cc1nnc(-c2ccccc2F)s1 ; O=C1C(=O)N(C2CCCC2)CCN1Cc1nnc(N2CC[C@H](F)C2)s1]

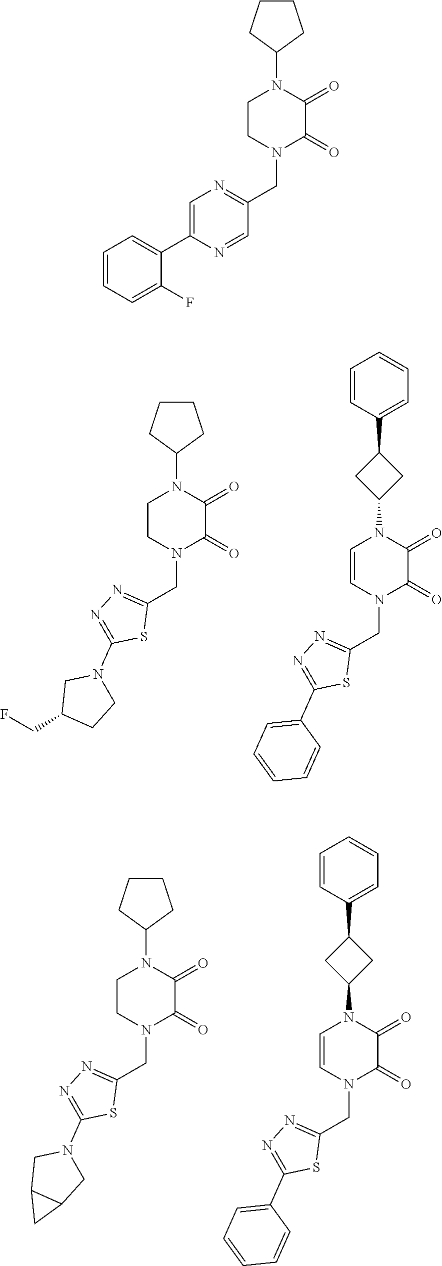 [SMILES: O=C1C(=O)N(C2CCCC2)CCN1Cc1cnc(-c2ccccc2F)cn1 ; O=C1C(=O)N(C2CCCC2)CCN1Cc1nnc(N2CC[C@H](CF)C2)s1 ; O=c1c(=O)n([C@H]2C[C@H](c3ccccc3)C2)ccn1Cc1nnc(-c2ccccc2)s1 ; O=C1C(=O)N(C2CCCC2)CCN1Cc1nnc(N2CC3CC3C2)s1 ; O=c1c(=O)n([C@H]2C[C@@H](c3ccccc3)C2)ccn1Cc1nnc(-c2ccccc2)s1]

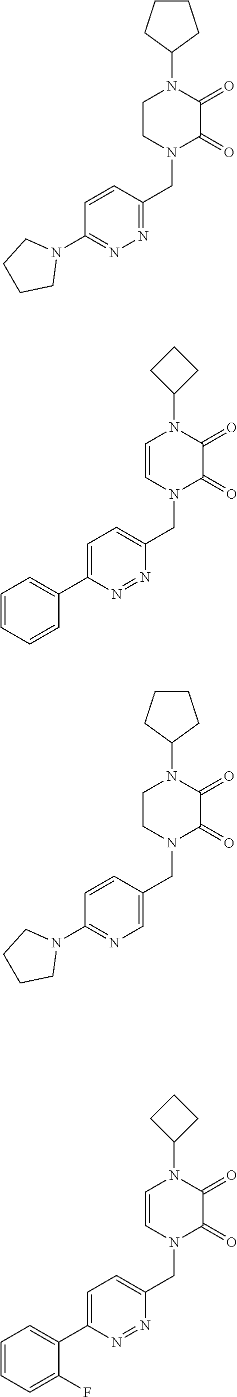 [SMILES: O=C1C(=O)N(C2CCCC2)CCN1Cc1ccc(N2CCCC2)nn1 ; O=c1c(=O)n(C2CCC2)ccn1Cc1ccc(-c2ccccc2)nn1 ; O=C1C(=O)N(C2CCCC2)CCN1Cc1ccc(N2CCCC2)nc1 ; O=c1c(=O)n(C2CCC2)ccn1Cc1ccc(-c2ccccc2F)nn1]

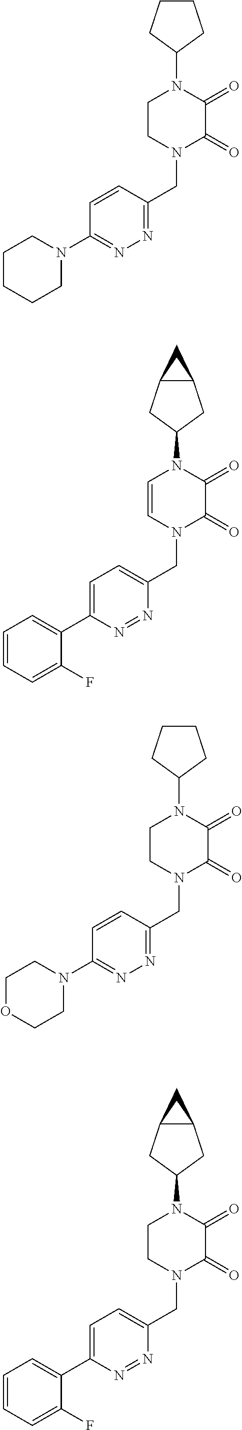 [SMILES: O=C1C(=O)N(C2CCCC2)CCN1Cc1ccc(N2CCCCC2)nn1 ; O=c1c(=O)n([C@H]2C[C@H]3C[C@H]3C2)ccn1Cc1ccc(-c2ccccc2F)nn1 ; O=C1C(=O)N(C2CCCC2)CCN1Cc1ccc(N2CCOCC2)nn1 ; O=C1C(=O)N([C@H]2C[C@H]3C[C@H]3C2)CCN1Cc1ccc(-c2ccccc2F)nn1]

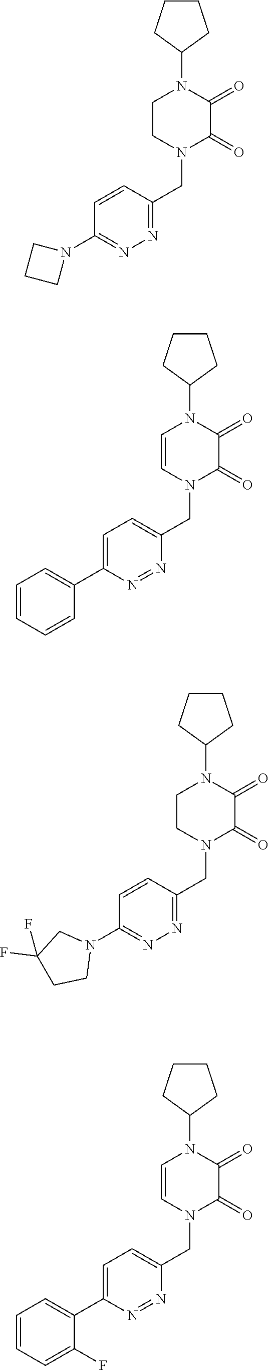 [SMILES: O=C1C(=O)N(C2CCCC2)CCN1Cc1ccc(N2CCC2)nn1 ; O=c1c(=O)n(C2CCCC2)ccn1Cc1ccc(-c2ccccc2)nn1 ; O=C1C(=O)N(C2CCCC2)CCN1Cc1ccc(N2CCC(F)(F)C2)nn1 ; O=c1c(=O)n(C2CCCC2)ccn1Cc1ccc(-c2ccccc2F)nn1]

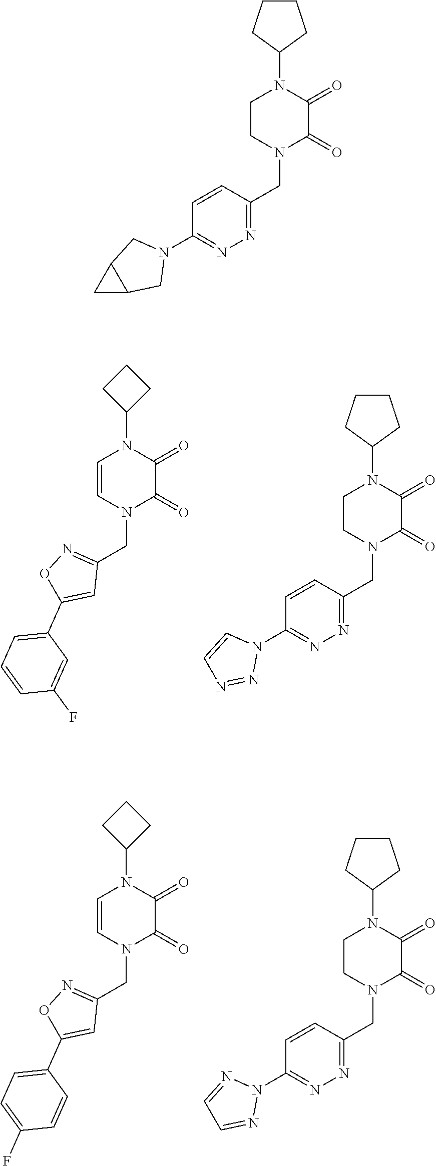 [SMILES: O=C1C(=O)N(C2CCCC2)CCN1Cc1ccc(N2CC3CC3C2)nn1 ; O=c1c(=O)n(C2CCC2)ccn1Cc1cc(-c2cccc(F)c2)on1 ; O=C1C(=O)N(C2CCCC2)CCN1Cc1ccc(-n2ccnn2)nn1 ; O=c1c(=O)n(C2CCC2)ccn1Cc1cc(-c2ccc(F)cc2)on1 ; O=C1C(=O)N(C2CCCC2)CCN1Cc1ccc(-n2nccn2)nn1]

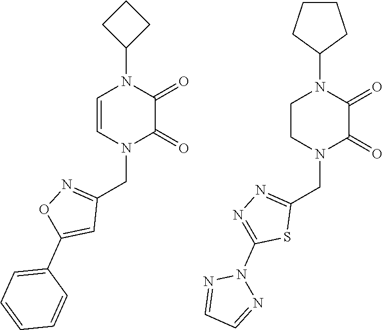 [SMILES: O=c1c(=O)n(C2CCC2)ccn1Cc1cc(-c2ccccc2)on1 ; O=C1C(=O)N(C2CCCC2)CCN1Cc1nnc(-n2nccn2)s1]

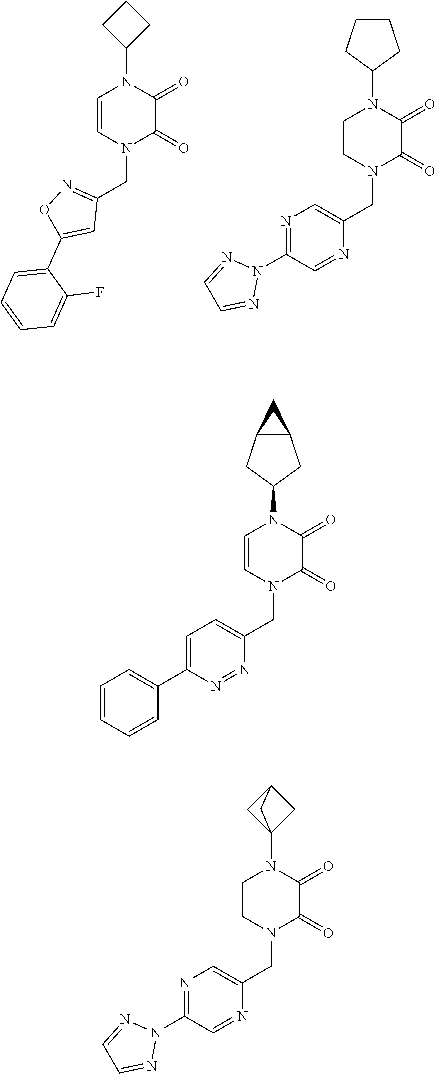 [SMILES: O=c1c(=O)n(C2CCC2)ccn1Cc1cc(-c2ccccc2F)on1 ; O=C1C(=O)N(C2CCCC2)CCN1Cc1cnc(-n2nccn2)cn1 ; O=c1c(=O)n([C@H]2C[C@H]3C[C@H]3C2)ccn1Cc1ccc(-c2ccccc2)nn1 ; O=C1C(=O)N(C23CC(C2)C3)CCN1Cc1cnc(-n2nccn2)cn1]

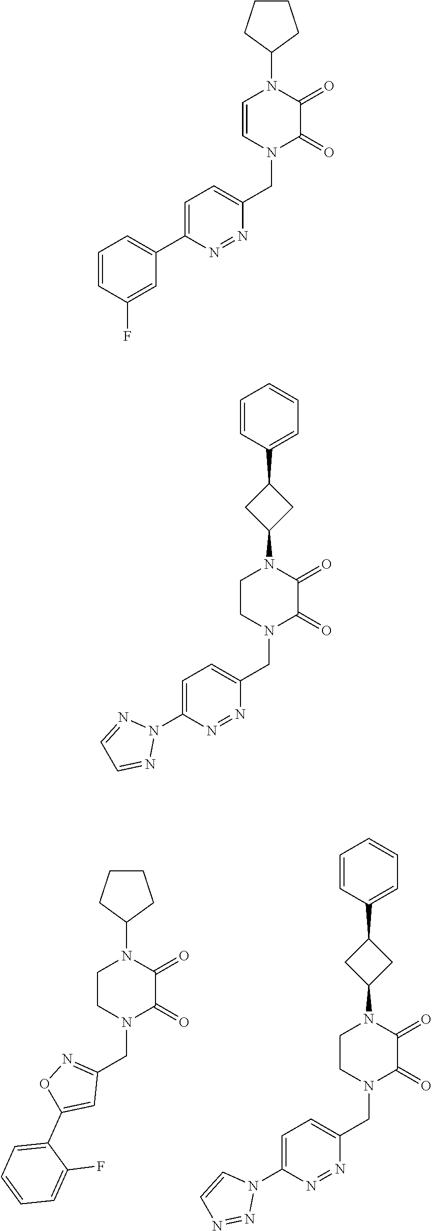 [SMILES: O=c1c(=O)n(C2CCCC2)ccn1Cc1ccc(-c2cccc(F)c2)nn1 ; O=C1C(=O)N([C@H]2C[C@@H](c3ccccc3)C2)CCN1Cc1ccc(-n2nccn2)nn1 ; O=C1C(=O)N(C2CCCC2)CCN1Cc1cc(-c2ccccc2F)on1 ; O=C1C(=O)N([C@H]2C[C@@H](c3ccccc3)C2)CCN1Cc1ccc(-n2ccnn2)nn1]

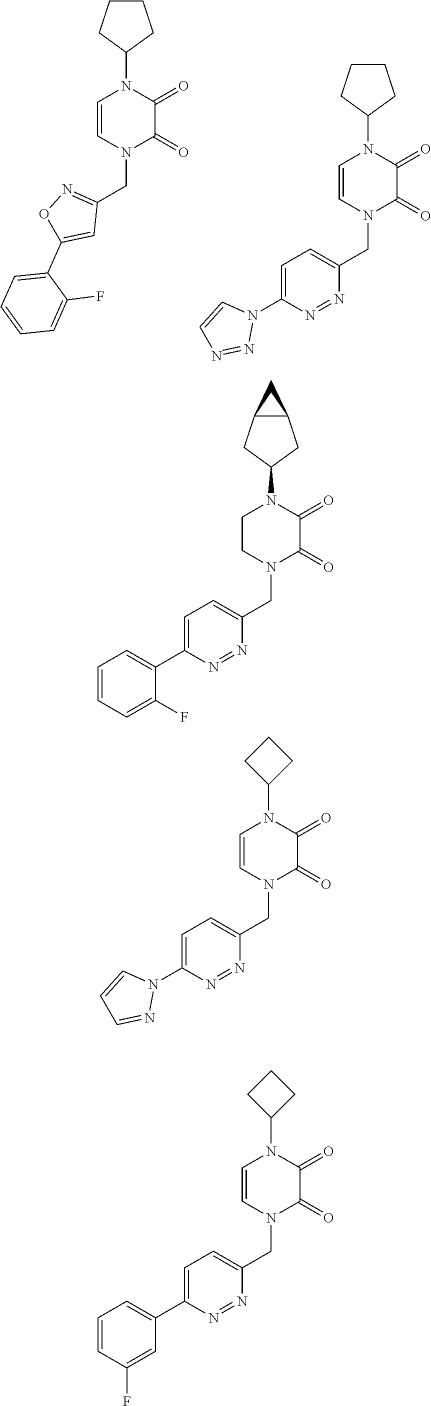

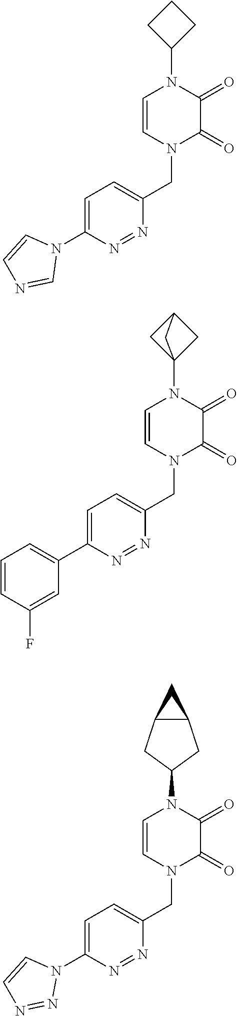 [SMILES: O=c1c(=O)n(C2CCC2)ccn1Cc1ccc(-n2ccnc2)nn1 ; O=c1c(=O)n(C23CC(C2)C3)ccn1Cc1ccc(-c2cccc(F)c2)nn1 ; O=c1c(=O)n([C@H]2C[C@H]3C[C@H]3C2)ccn1Cc1ccc(-n2ccnn2)nn1]

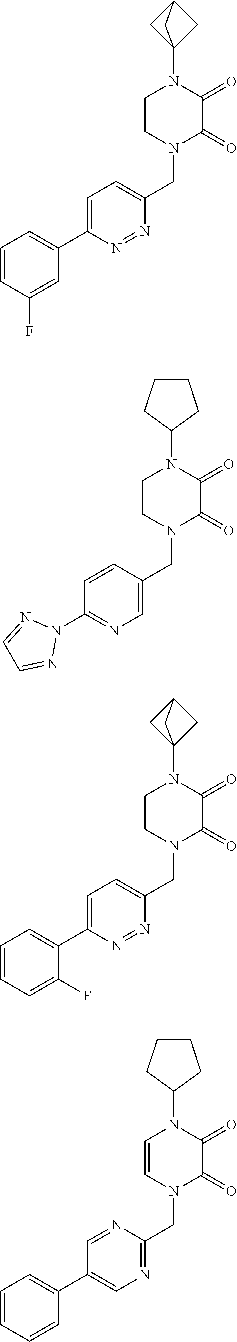 [SMILES: O=C1C(=O)N(C23CC(C2)C3)CCN1Cc1ccc(-c2cccc(F)c2)nn1 ; O=C1C(=O)N(C2CCCC2)CCN1Cc1ccc(-n2nccn2)nc1 ; O=C1C(=O)N(C23CC(C2)C3)CCN1Cc1ccc(-c2ccccc2F)nn1 ; O=c1c(=O)n(C2CCCC2)ccn1Cc1ncc(-c2ccccc2)cn1]

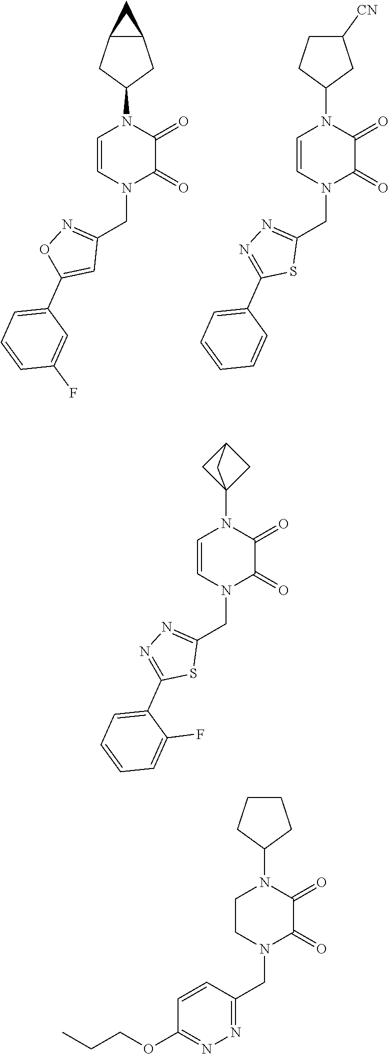 [SMILES: O=c1c(=O)n([C@H]2C[C@H]3C[C@H]3C2)ccn1Cc1cc(-c2cccc(F)c2)on1 ; N#CC1CCC(n2ccn(Cc3nnc(-c4ccccc4)s3)c(=O)c2=O)C1 ; O=c1c(=O)n(C23CC(C2)C3)ccn1Cc1nnc(-c2ccccc2F)s1 ; CCCOc1ccc(CN2CCN(C3CCCC3)C(=O)C2=O)nn1]

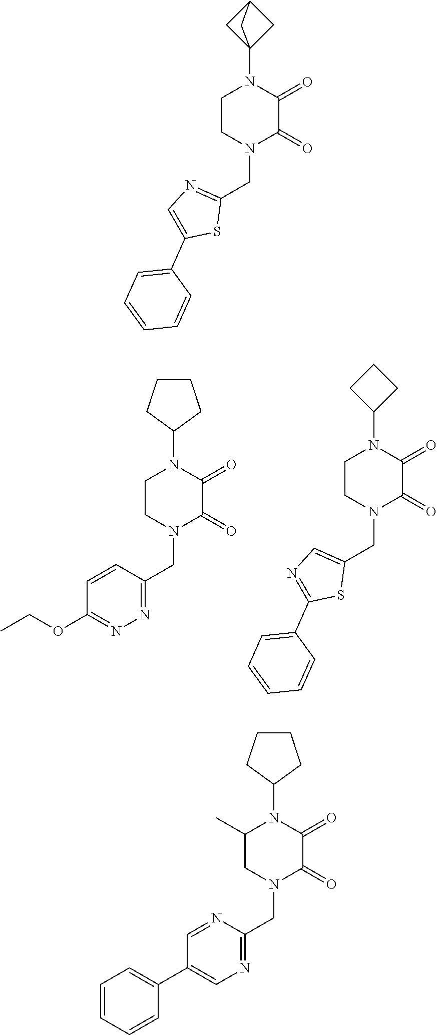

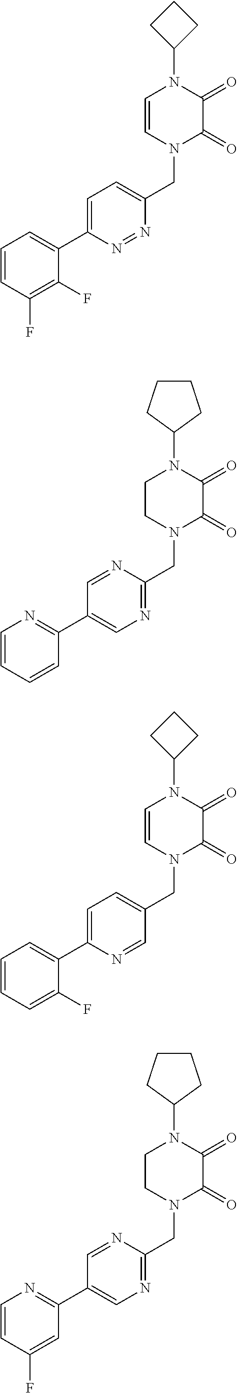 [SMILES: O=c1c(=O)n(C2CCC2)ccn1Cc1ccc(-c2cccc(F)c2F)nn1 ; O=C1C(=O)N(C2CCCC2)CCN1Cc1ncc(-c2ccccn2)cn1 ; O=c1c(=O)n(C2CCC2)ccn1Cc1ccc(-c2ccccc2F)nc1 ; O=C1C(=O)N(C2CCCC2)CCN1Cc1ncc(-c2cc(F)ccn2)cn1]

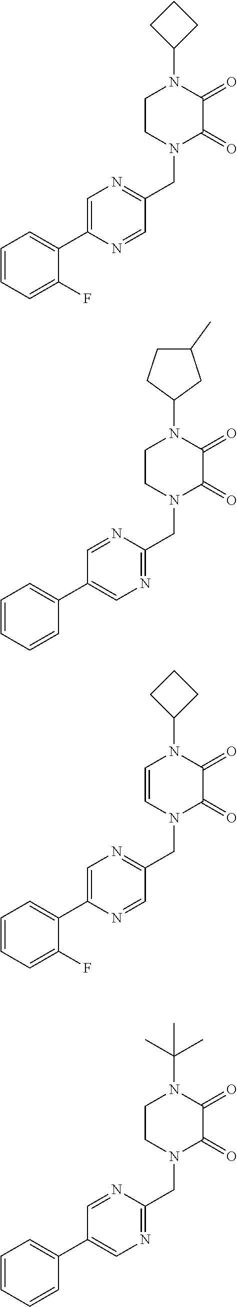 [SMILES: O=C1C(=O)N(C2CCC2)CCN1Cc1cnc(-c2ccccc2F)cn1 ; CC1CCC(N2CCN(Cc3ncc(-c4ccccc4)cn3)C(=O)C2=O)C1 ; O=c1c(=O)n(C2CCC2)ccn1Cc1cnc(-c2ccccc2F)cn1 ; CC(C)(C)N1CCN(Cc2ncc(-c3ccccc3)cn2)C(=O)C1=O]

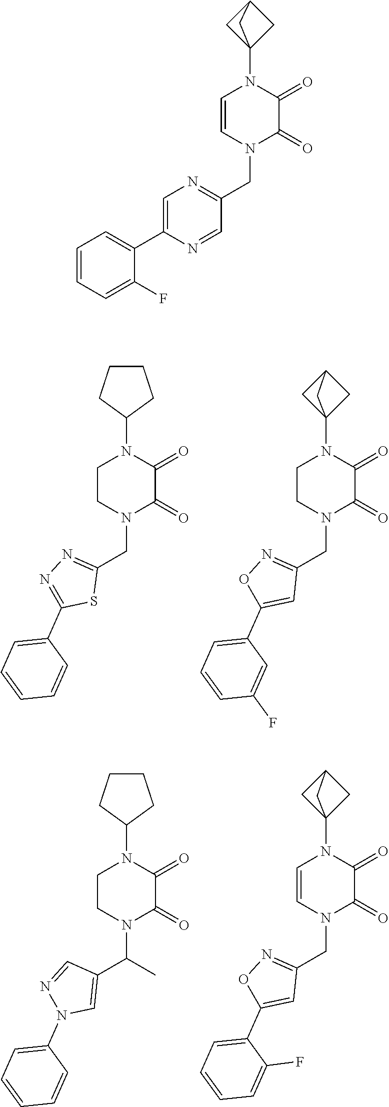 [SMILES: O=c1c(=O)n(C23CC(C2)C3)ccn1Cc1cnc(-c2ccccc2F)cn1 ; O=C1C(=O)N(C2CCCC2)CCN1Cc1nnc(-c2ccccc2)s1 ; O=C1C(=O)N(C23CC(C2)C3)CCN1Cc1cc(-c2cccc(F)c2)on1 ; CC(c1cnn(-c2ccccc2)c1)N1CCN(C2CCCC2)C(=O)C1=O ; O=c1c(=O)n(C23CC(C2)C3)ccn1Cc1cc(-c2ccccc2F)on1]

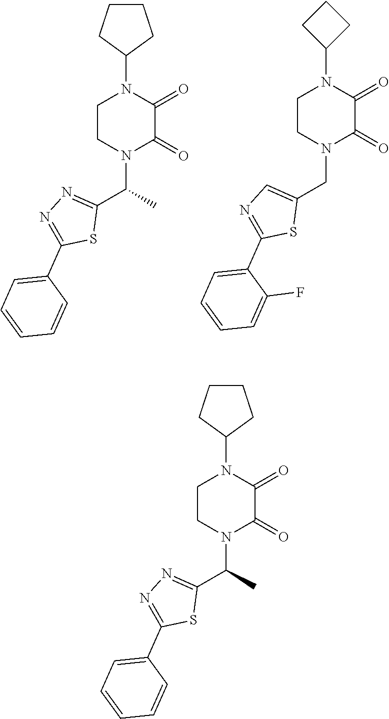 [SMILES: C[C@H](c1nnc(-c2ccccc2)s1)N1CCN(C2CCCC2)C(=O)C1=O ; O=C1C(=O)N(C2CCC2)CCN1Cc1cnc(-c2ccccc2F)s1 ; C[C@@H](c1nnc(-c2ccccc2)s1)N1CCN(C2CCCC2)C(=O)C1=O]

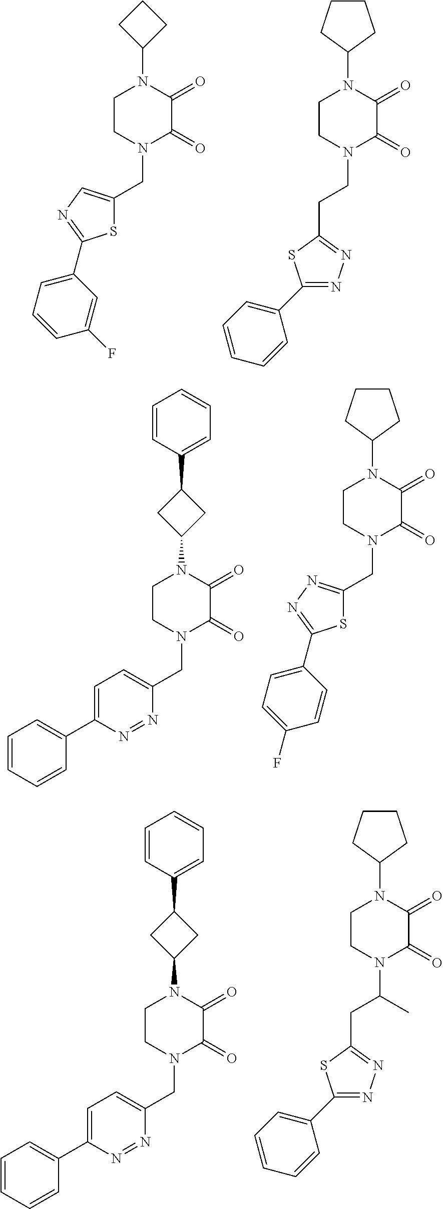 [SMILES: O=C1C(=O)N(C2CCC2)CCN1Cc1cnc(-c2cccc(F)c2)s1 ; O=C1C(=O)N(C2CCCC2)CCN1CCc1nnc(-c2ccccc2)s1 ; O=C1C(=O)N([C@H]2C[C@H](c3ccccc3)C2)CCN1Cc1ccc(-c2ccccc2)nn1 ; O=C1C(=O)N(C2CCCC2)CCN1Cc1nnc(-c2ccc(F)cc2)s1 ; O=C1C(=O)N([C@H]2C[C@@H](c3ccccc3)C2)CCN1Cc1ccc(-c2ccccc2)nn1 ; CC(Cc1nnc(-c2ccccc2)s1)N1CCN(C2CCCC2)C(=O)C1=O]

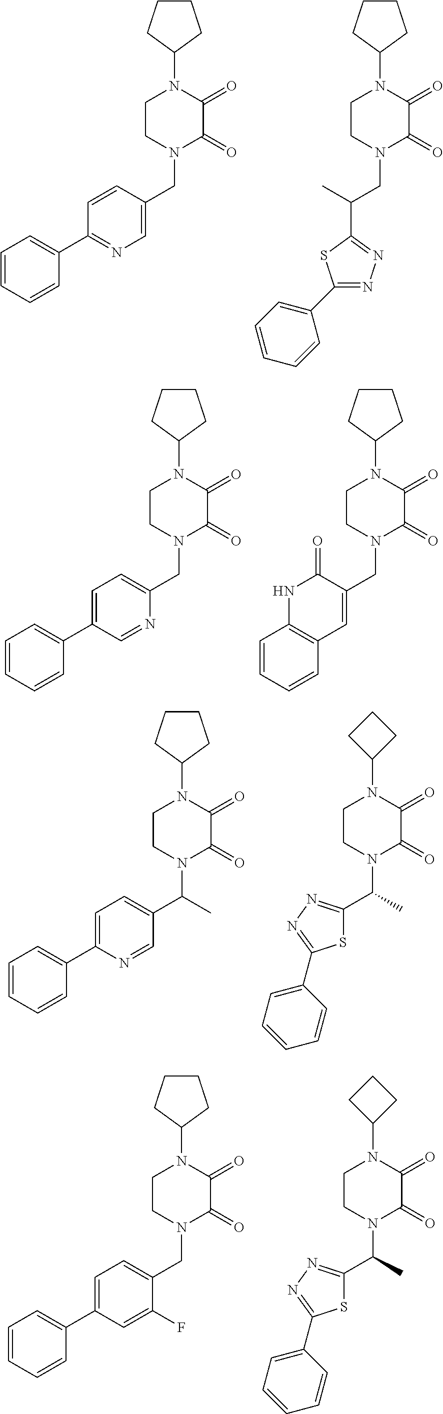

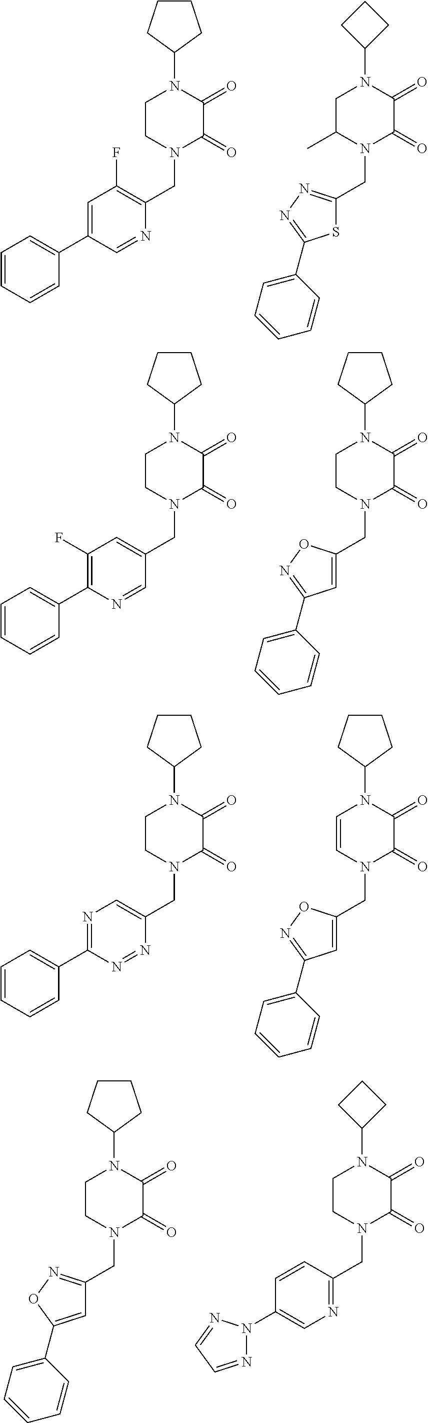

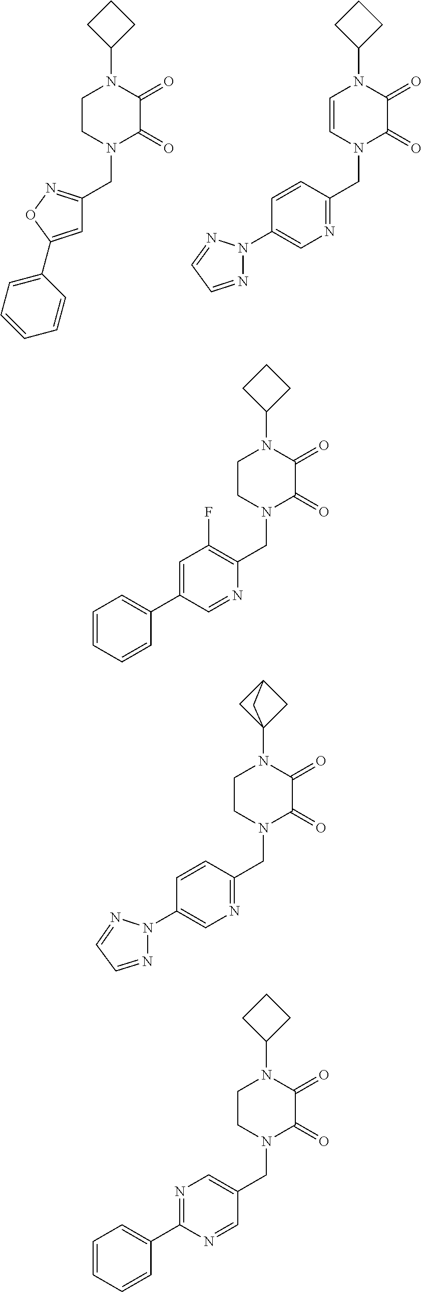 [SMILES: O=C1C(=O)N(C2CCC2)CCN1Cc1cc(-c2ccccc2)on1 ; O=c1c(=O)n(C2CCC2)ccn1Cc1ccc(-n2nccn2)cn1 ; O=C1C(=O)N(C2CCC2)CCN1Cc1ncc(-c2ccccc2)cc1F ; O=C1C(=O)N(C23CC(C2)C3)CCN1Cc1ccc(-n2nccn2)cn1 ; O=C1C(=O)N(C2CCC2)CCN1Cc1cnc(-c2ccccc2)nc1]

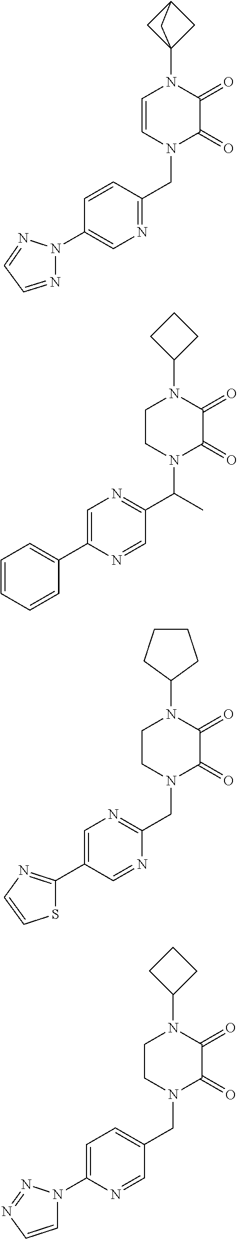 [SMILES: O=c1c(=O)n(C23CC(C2)C3)ccn1Cc1ccc(-n2nccn2)cn1 ; CC(c1cnc(-c2ccccc2)cn1)N1CCN(C2CCC2)C(=O)C1=O ; O=C1C(=O)N(C2CCCC2)CCN1Cc1ncc(-c2nccs2)cn1 ; O=C1C(=O)N(C2CCC2)CCN1Cc1ccc(-n2ccnn2)nc1]

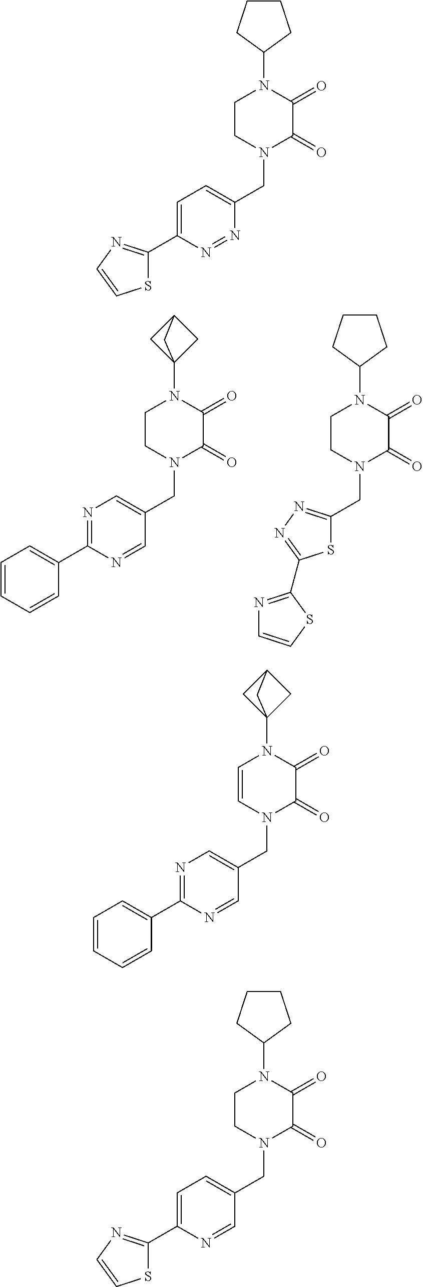

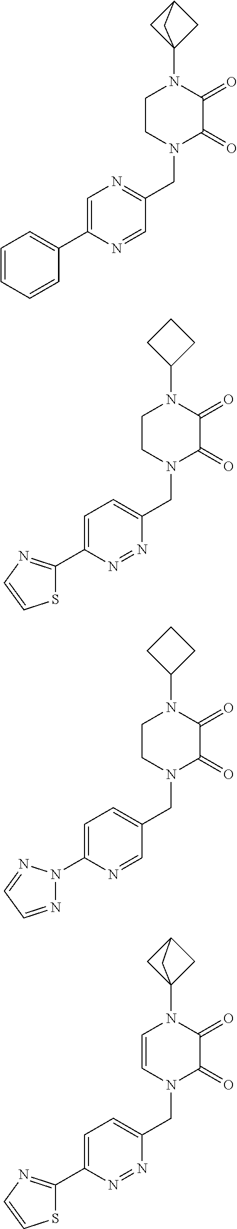 [SMILES: O=C1C(=O)N(C23CC(C2)C3)CCN1Cc1cnc(-c2ccccc2)cn1 ; O=C1C(=O)N(C2CCC2)CCN1Cc1ccc(-c2nccs2)nn1 ; O=C1C(=O)N(C2CCC2)CCN1Cc1ccc(-n2nccn2)nc1 ; O=c1c(=O)n(C23CC(C2)C3)ccn1Cc1ccc(-c2nccs2)nn1]

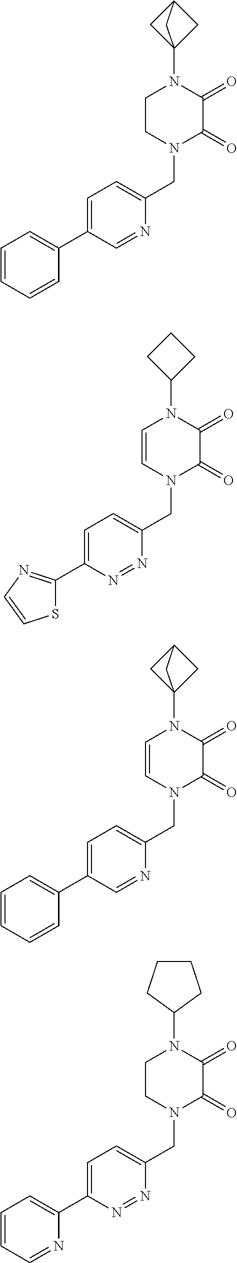 [SMILES: O=C1C(=O)N(C23CC(C2)C3)CCN1Cc1ccc(-c2ccccc2)cn1 ; O=c1c(=O)n(C2CCC2)ccn1Cc1ccc(-c2nccs2)nn1 ; O=c1c(=O)n(C23CC(C2)C3)ccn1Cc1ccc(-c2ccccc2)cn1 ; O=C1C(=O)N(C2CCCC2)CCN1Cc1ccc(-c2ccccn2)nn1]

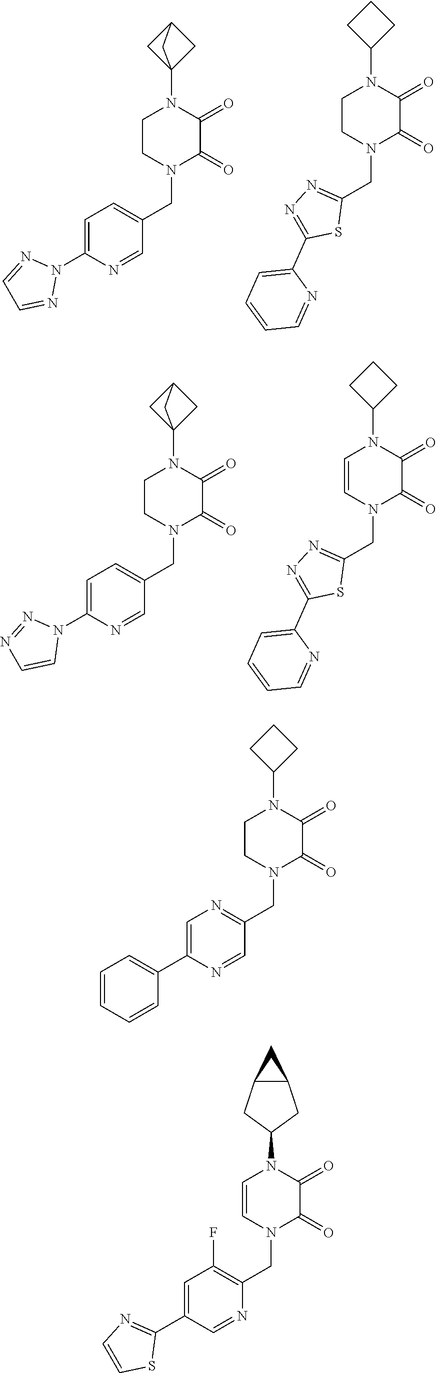

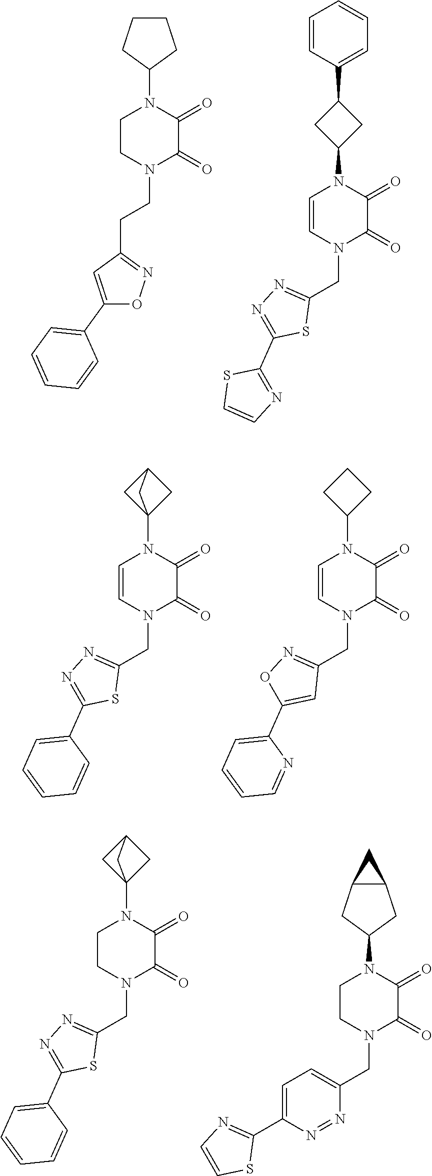 [SMILES: O=C1C(=O)N(C2CCCC2)CCN1CCc1cc(-c2ccccc2)on1 ; O=c1c(=O)n([C@H]2C[C@@H](c3ccccc3)C2)ccn1Cc1nnc(-c2nccs2)s1 ; O=c1c(=O)n(C23CC(C2)C3)ccn1Cc1nnc(-c2ccccc2)s1 ; O=c1c(=O)n(C2CCC2)ccn1Cc1cc(-c2ccccn2)on1 ; O=C1C(=O)N(C23CC(C2)C3)CCN1Cc1nnc(-c2ccccc2)s1 ; O=C1C(=O)N([C@H]2C[C@H]3C[C@H]3C2)CCN1Cc1ccc(-c2nccs2)nn1]

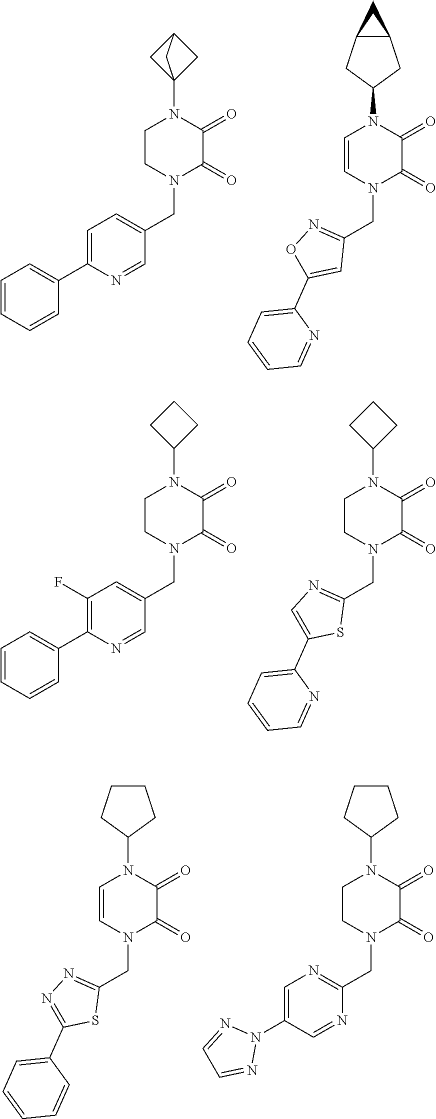 [SMILES: O=C1C(=O)N(C23CC(C2)C3)CCN1Cc1ccc(-c2ccccc2)nc1 ; O=c1c(=O)n([C@H]2C[C@H]3C[C@H]3C2)ccn1Cc1cc(-c2ccccn2)on1 ; O=C1C(=O)N(C2CCC2)CCN1Cc1cnc(-c2ccccc2)c(F)c1 ; O=C1C(=O)N(C2CCC2)CCN1Cc1ncc(-c2ccccn2)s1 ; O=c1c(=O)n(C2CCCC2)ccn1Cc1nnc(-c2ccccc2)s1 ; O=C1C(=O)N(C2CCCC2)CCN1Cc1ncc(-n2nccn2)cn1]

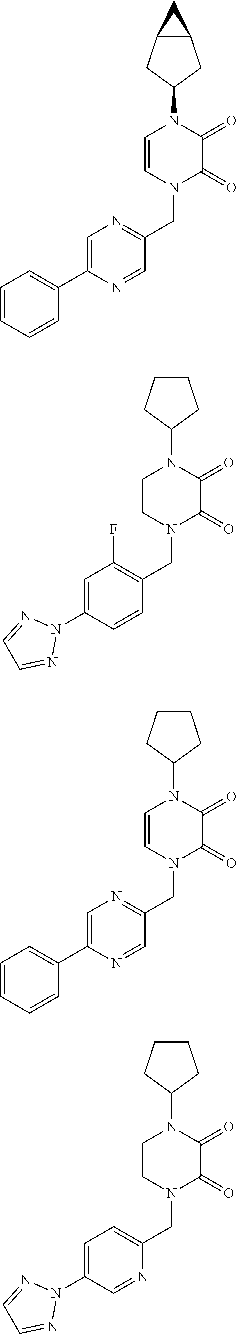 [SMILES: O=c1c(=O)n([C@H]2C[C@H]3C[C@H]3C2)ccn1Cc1cnc(-c2ccccc2)cn1 ; O=C1C(=O)N(C2CCCC2)CCN1Cc1ccc(-n2nccn2)cc1F ; O=c1c(=O)n(C2CCCC2)ccn1Cc1cnc(-c2ccccc2)cn1 ; O=C1C(=O)N(C2CCCC2)CCN1Cc1ccc(-n2nccn2)cn1]

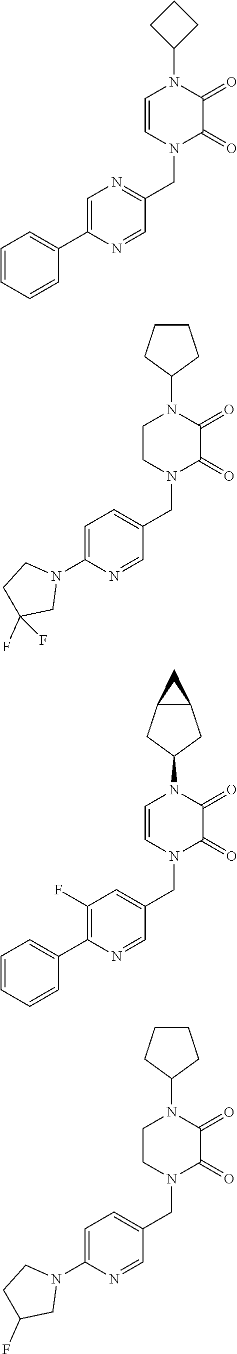 [SMILES: O=c1c(=O)n(C2CCC2)ccn1Cc1cnc(-c2ccccc2)cn1 ; O=C1C(=O)N(C2CCCC2)CCN1Cc1ccc(N2CCC(F)(F)C2)nc1 ; O=c1c(=O)n([C@H]2C[C@H]3C[C@H]3C2)ccn1Cc1cnc(-c2ccccc2)c(F)c1 ; O=C1C(=O)N(C2CCCC2)CCN1Cc1ccc(N2CCC(F)C2)nc1]

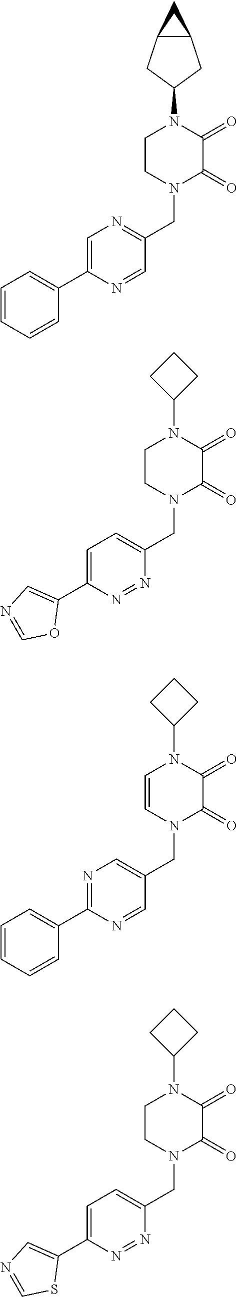 [SMILES: O=C1C(=O)N([C@H]2C[C@H]3C[C@H]3C2)CCN1Cc1cnc(-c2ccccc2)cn1 ; O=C1C(=O)N(C2CCC2)CCN1Cc1ccc(-c2cnco2)nn1 ; O=c1c(=O)n(C2CCC2)ccn1Cc1cnc(-c2ccccc2)nc1 ; O=C1C(=O)N(C2CCC2)CCN1Cc1ccc(-c2cncs2)nn1]

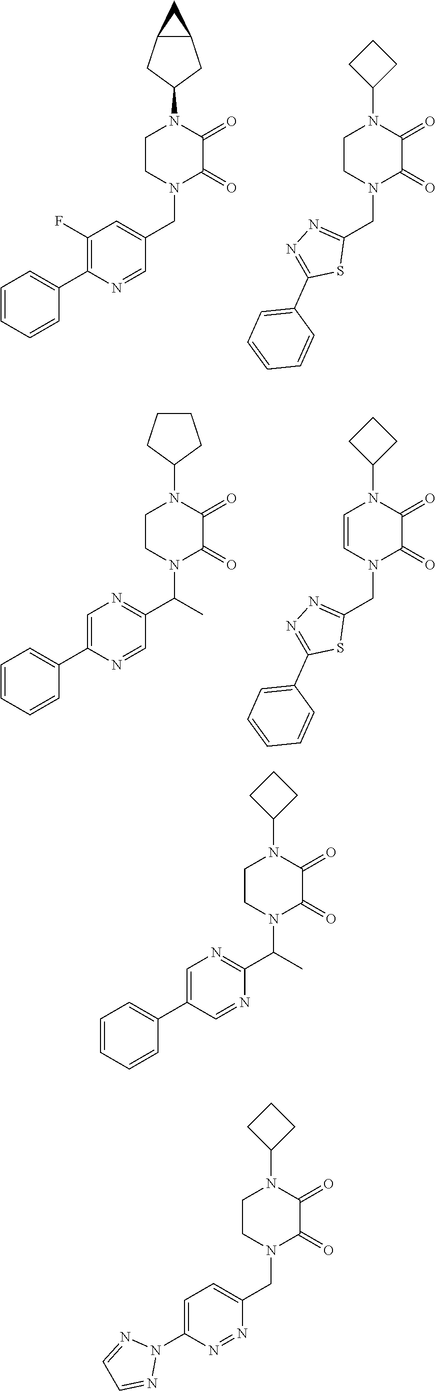

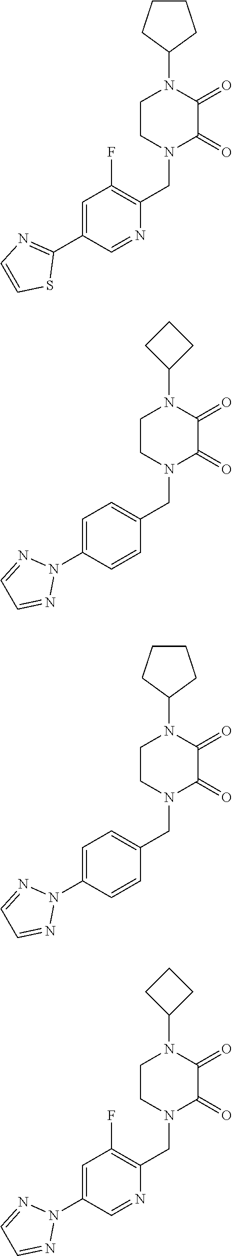 [SMILES: O=C1C(=O)N(C2CCCC2)CCN1Cc1ncc(-c2nccs2)cc1F ; O=C1C(=O)N(C2CCC2)CCN1Cc1ccc(-n2nccn2)cc1 ; O=C1C(=O)N(C2CCCC2)CCN1Cc1ccc(-n2nccn2)cc1 ; O=C1C(=O)N(C2CCC2)CCN1Cc1ncc(-n2nccn2)cc1F]

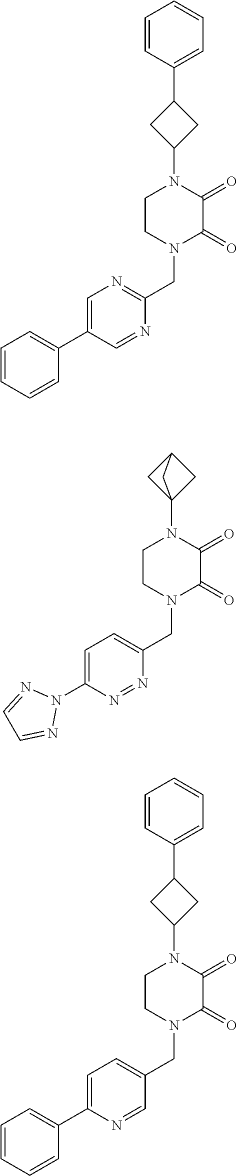 [SMILES: O=C1C(=O)N(C2CC(c3ccccc3)C2)CCN1Cc1ncc(-c2ccccc2)cn1 ; O=C1C(=O)N(C23CC(C2)C3)CCN1Cc1ccc(-n2nccn2)nn1 ; O=C1C(=O)N(C2CC(c3ccccc3)C2)CCN1Cc1ccc(-c2ccccc2)nc1]

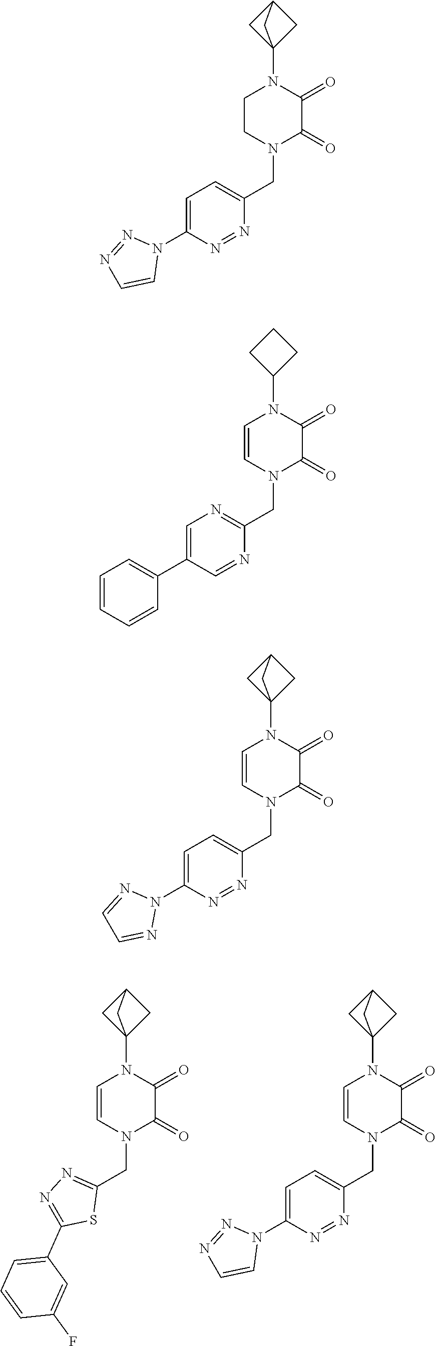 [SMILES: O=C1C(=O)N(C23CC(C2)C3)CCN1Cc1ccc(-n2ccnn2)nn1 ; O=c1c(=O)n(C2CCC2)ccn1Cc1ncc(-c2ccccc2)cn1 ; O=c1c(=O)n(C23CC(C2)C3)ccn1Cc1ccc(-n2nccn2)nn1 ; O=c1c(=O)n(C23CC(C2)C3)ccn1Cc1nnc(-c2cccc(F)c2)s1 ; O=c1c(=O)n(C23CC(C2)C3)ccn1Cc1ccc(-n2ccnn2)nn1]

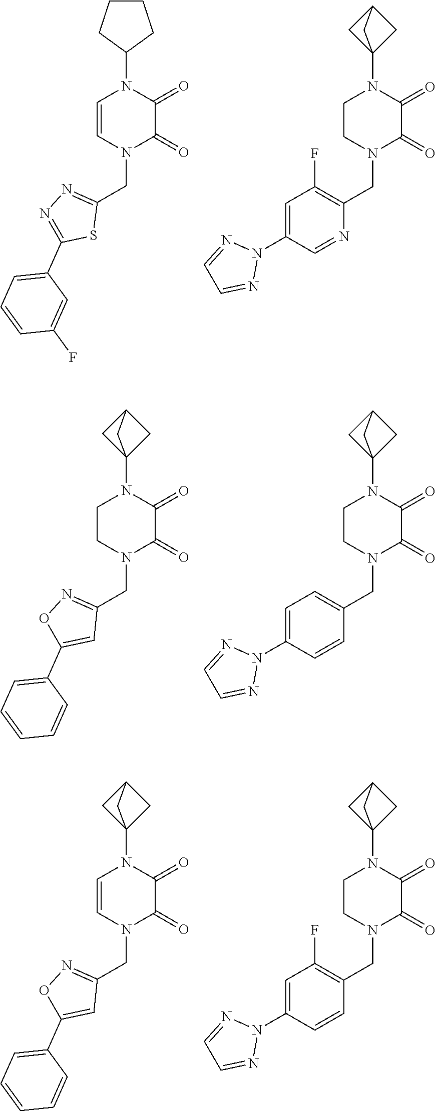 [SMILES: O=c1c(=O)n(C2CCCC2)ccn1Cc1nnc(-c2cccc(F)c2)s1 ; O=C1C(=O)N(C23CC(C2)C3)CCN1Cc1ncc(-n2nccn2)cc1F ; O=C1C(=O)N(C23CC(C2)C3)CCN1Cc1cc(-c2ccccc2)on1 ; O=C1C(=O)N(C23CC(C2)C3)CCN1Cc1ccc(-n2nccn2)cc1 ; O=c1c(=O)n(C23CC(C2)C3)ccn1Cc1cc(-c2ccccc2)on1 ; O=C1C(=O)N(C23CC(C2)C3)CCN1Cc1ccc(-n2nccn2)cc1F]

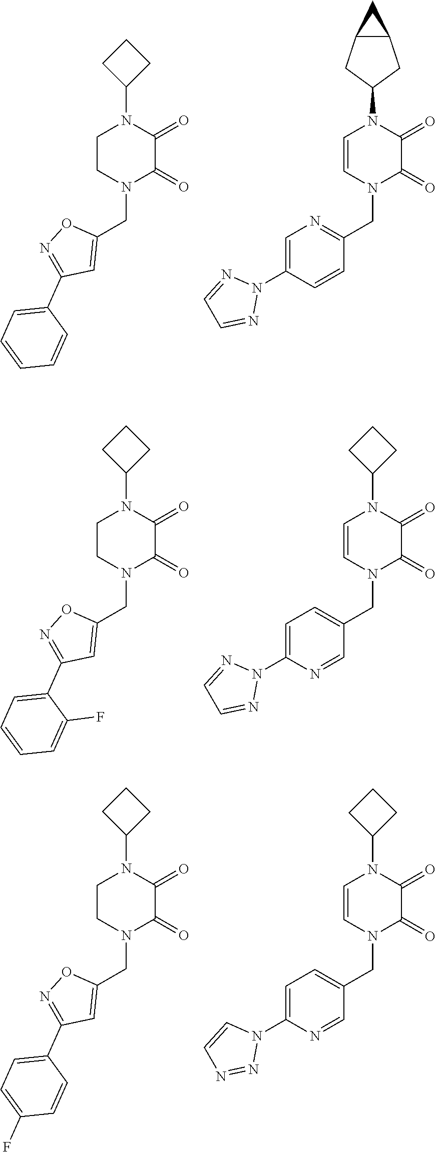 [SMILES: O=C1C(=O)N(C2CCC2)CCN1Cc1cc(-c2ccccc2)no1 ; O=c1c(=O)n([C@H]2C[C@H]3C[C@H]3C2)ccn1Cc1ccc(-n2nccn2)cn1 ; O=C1C(=O)N(C2CCC2)CCN1Cc1cc(-c2ccccc2F)no1 ; O=c1c(=O)n(C2CCC2)ccn1Cc1ccc(-n2nccn2)nc1 ; O=C1C(=O)N(C2CCC2)CCN1Cc1cc(-c2ccc(F)cc2)no1 ; O=c1c(=O)n(C2CCC2)ccn1Cc1ccc(-n2ccnn2)nc1]

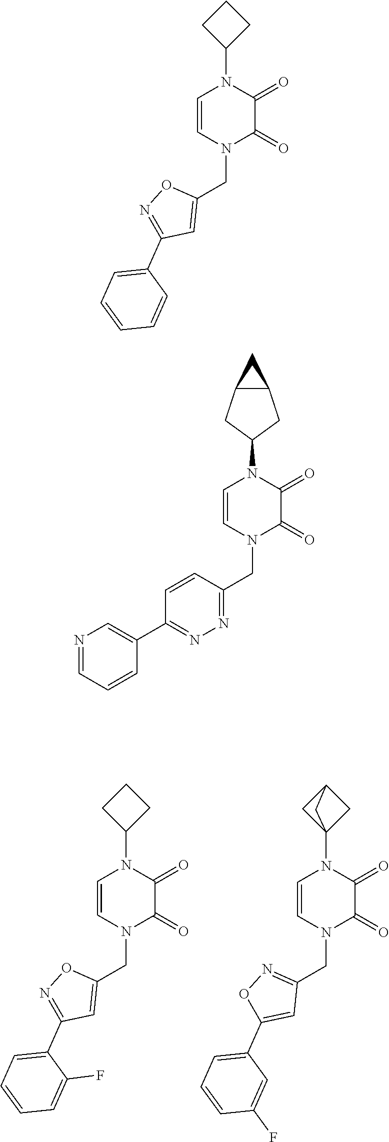 [SMILES: O=c1c(=O)n(C2CCC2)ccn1Cc1cc(-c2ccccc2)no1 ; O=c1c(=O)n([C@H]2C[C@H]3C[C@H]3C2)ccn1Cc1ccc(-c2cccnc2)nn1 ; O=c1c(=O)n(C2CCC2)ccn1Cc1cc(-c2ccccc2F)no1 ; O=c1c(=O)n(C23CC(C2)C3)ccn1Cc1cc(-c2cccc(F)c2)on1]

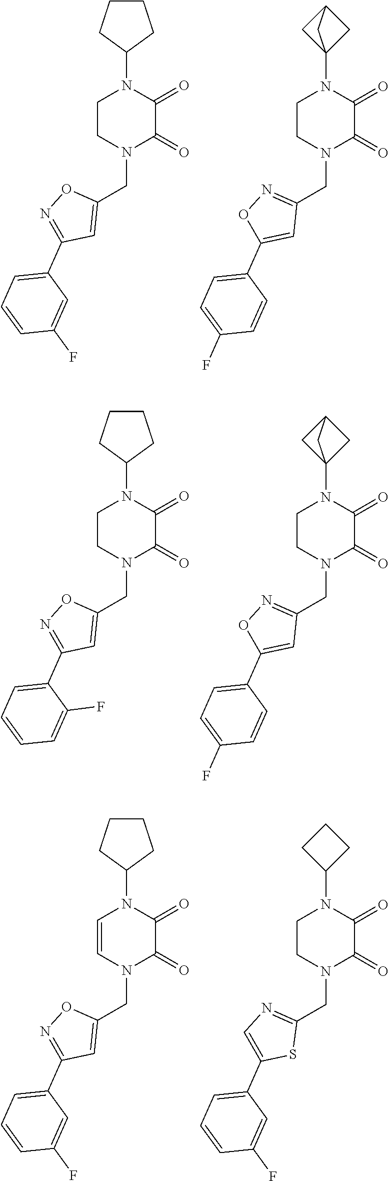 [SMILES: O=C1C(=O)N(C2CCCC2)CCN1Cc1cc(-c2cccc(F)c2)no1 ; O=C1C(=O)N(C23CC(C2)C3)CCN1Cc1cc(-c2ccc(F)cc2)on1 ; O=C1C(=O)N(C2CCCC2)CCN1Cc1cc(-c2ccccc2F)no1 ; O=C1C(=O)N(C23CC(C2)C3)CCN1Cc1cc(-c2ccc(F)cc2)on1 ; O=c1c(=O)n(C2CCCC2)ccn1Cc1cc(-c2cccc(F)c2)no1 ; O=C1C(=O)N(C2CCC2)CCN1Cc1ncc(-c2cccc(F)c2)s1]

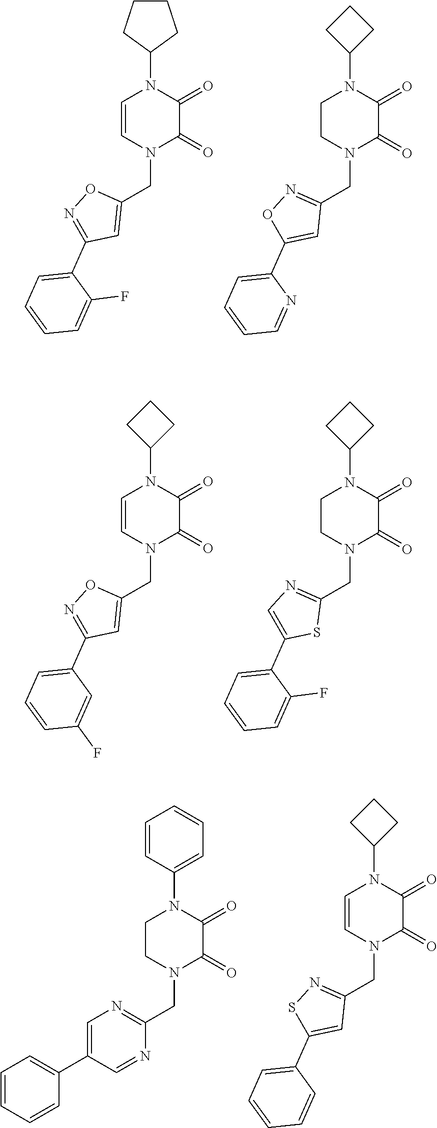 [SMILES: O=c1c(=O)n(C2CCCC2)ccn1Cc1cc(-c2ccccc2F)no1 ; O=C1C(=O)N(C2CCC2)CCN1Cc1cc(-c2ccccn2)on1 ; O=c1c(=O)n(C2CCC2)ccn1Cc1cc(-c2cccc(F)c2)no1 ; O=C1C(=O)N(C2CCC2)CCN1Cc1ncc(-c2ccccc2F)s1 ; O=C1C(=O)N(c2ccccc2)CCN1Cc1ncc(-c2ccccc2)cn1 ; O=c1c(=O)n(C2CCC2)ccn1Cc1cc(-c2ccccc2)sn1]

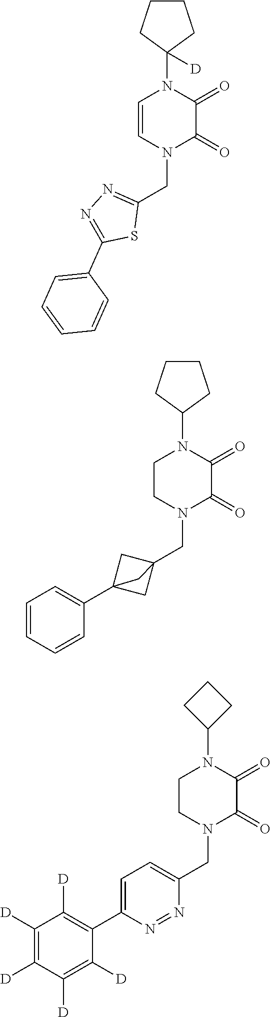 [SMILES: [2H]C1(n2ccn(Cc3nnc(-c4ccccc4)s3)c(=O)c2=O)CCCC1 ; O=C1C(=O)N(C2CCCC2)CCN1CC12CC(c3ccccc3)(C1)C2 ; [2H]c1c([2H])c([2H])c(-c2ccc(CN3CCN(C4CCC4)C(=O)C3=O)nn2)c([2H])c1[2H]]

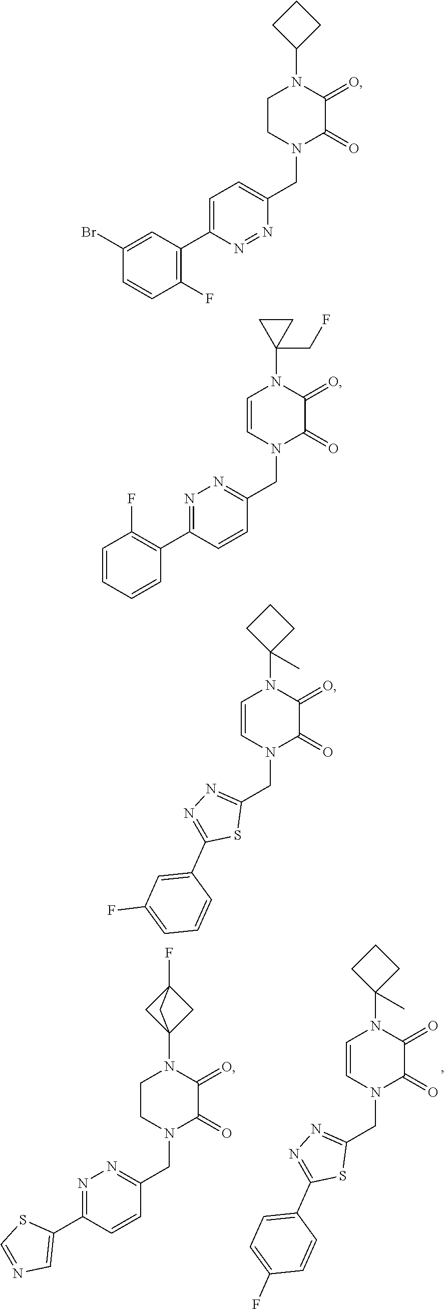

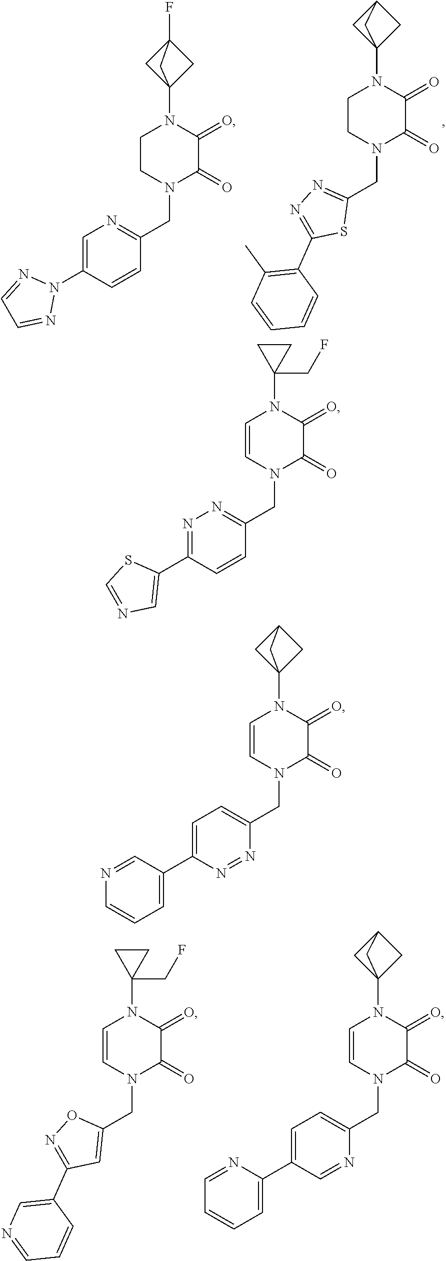

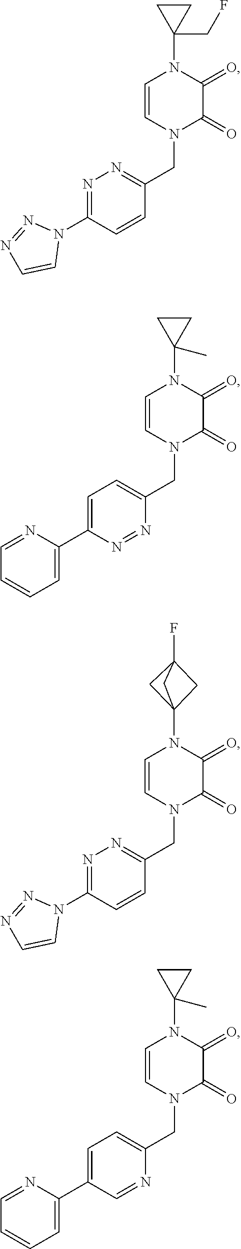 [SMILES: O=c1c(=O)n(C2(CF)CC2)ccn1Cc1ccc(-n2ccnn2)nn1 ; CC1(n2ccn(Cc3ccc(-c4ccccn4)nn3)c(=O)c2=O)CC1 ; O=c1c(=O)n(C23CC(F)(C2)C3)ccn1Cc1ccc(-n2ccnn2)nn1 ; CC1(n2ccn(Cc3ccc(-c4ccccn4)cn3)c(=O)c2=O)CC1]

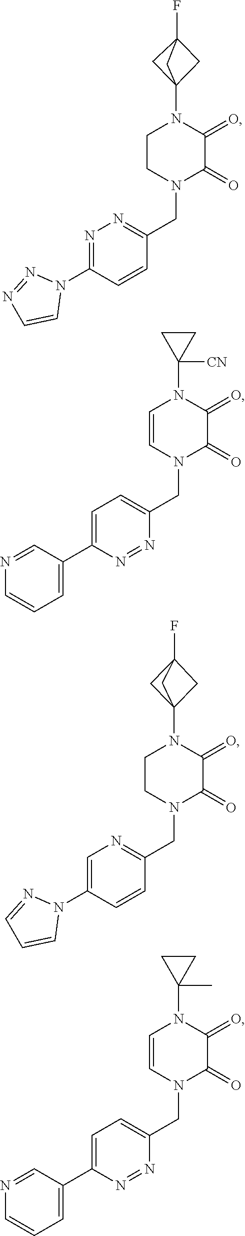 [SMILES: O=C1C(=O)N(C23CC(F)(C2)C3)CCN1Cc1ccc(-n2ccnn2)nn1 ; N#CC1(n2ccn(Cc3ccc(-c4cccnc4)nn3)c(=O)c2=O)CC1 ; O=C1C(=O)N(C23CC(F)(C2)C3)CCN1Cc1ccc(-n2cccn2)cn1 ; CC1(n2ccn(Cc3ccc(-c4cccnc4)nn3)c(=O)c2=O)CC1]

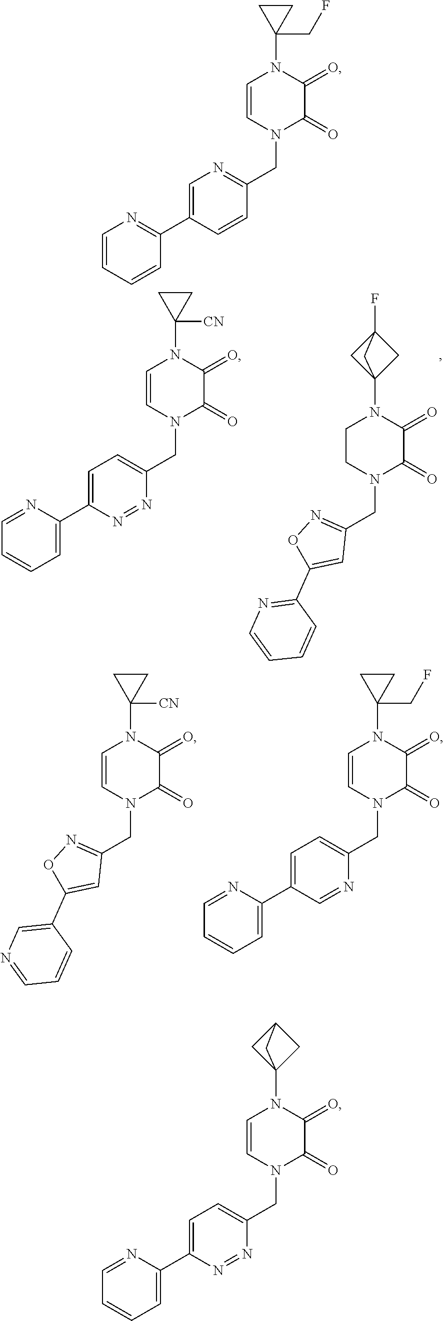

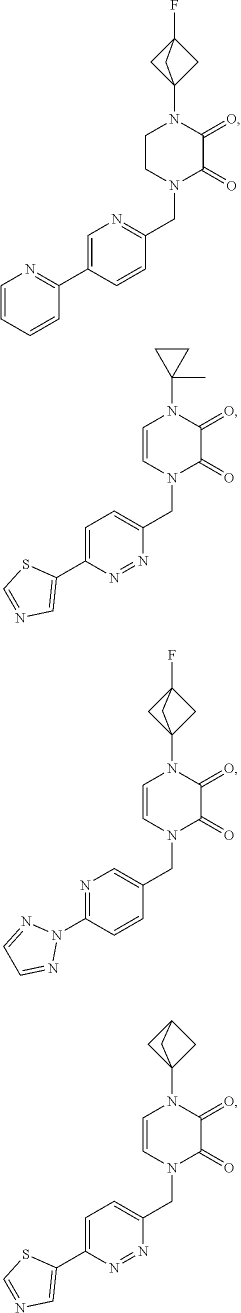 [SMILES: O=C1C(=O)N(C23CC(F)(C2)C3)CCN1Cc1ccc(-c2ccccn2)cn1 ; CC1(n2ccn(Cc3ccc(-c4cncs4)nn3)c(=O)c2=O)CC1 ; O=c1c(=O)n(C23CC(F)(C2)C3)ccn1Cc1ccc(-n2nccn2)nc1 ; O=c1c(=O)n(C23CC(C2)C3)ccn1Cc1ccc(-c2cncs2)nn1]

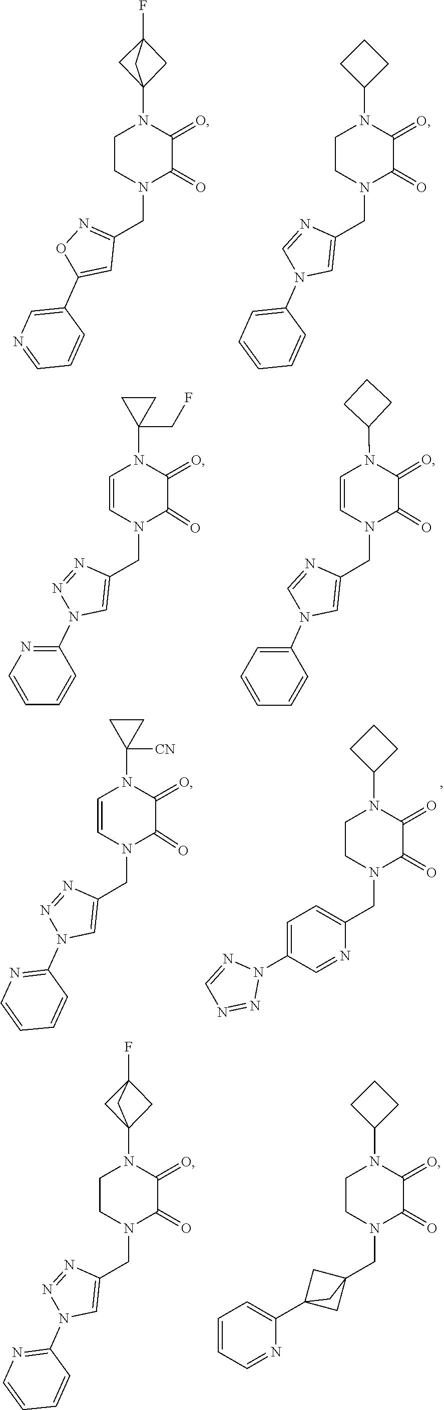

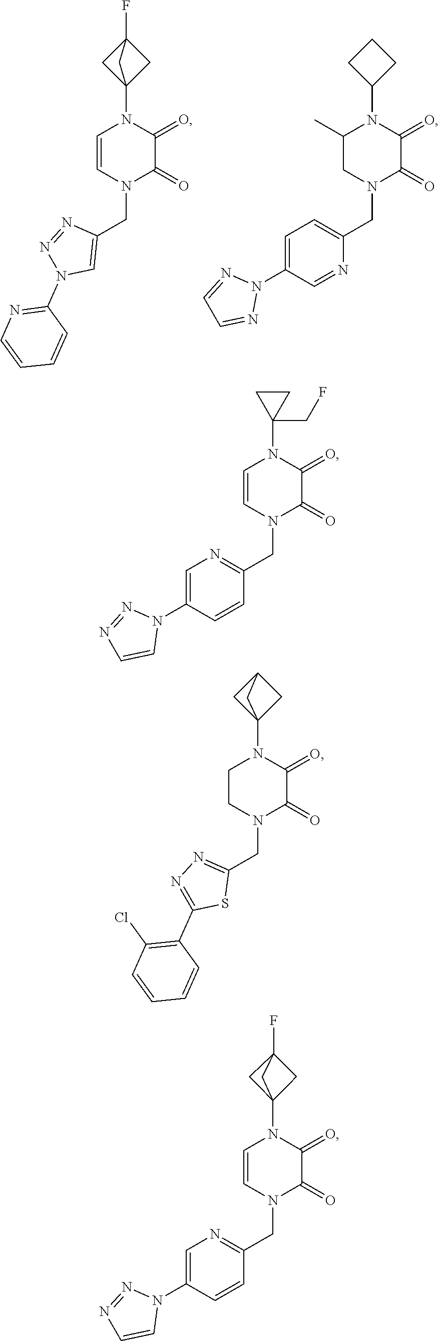 [SMILES: O=c1c(=O)n(C23CC(F)(C2)C3)ccn1Cc1cn(-c2ccccn2)nn1 ; CC1CN(Cc2ccc(-n3nccn3)cn2)C(=O)C(=O)N1C1CCC1 ; O=c1c(=O)n(C2(CF)CC2)ccn1Cc1ccc(-n2ccnn2)cn1 ; O=C1C(=O)N(C23CC(C2)C3)CCN1Cc1nnc(-c2ccccc2Cl)s1 ; O=c1c(=O)n(C23CC(F)(C2)C3)ccn1Cc1ccc(-n2ccnn2)cn1]

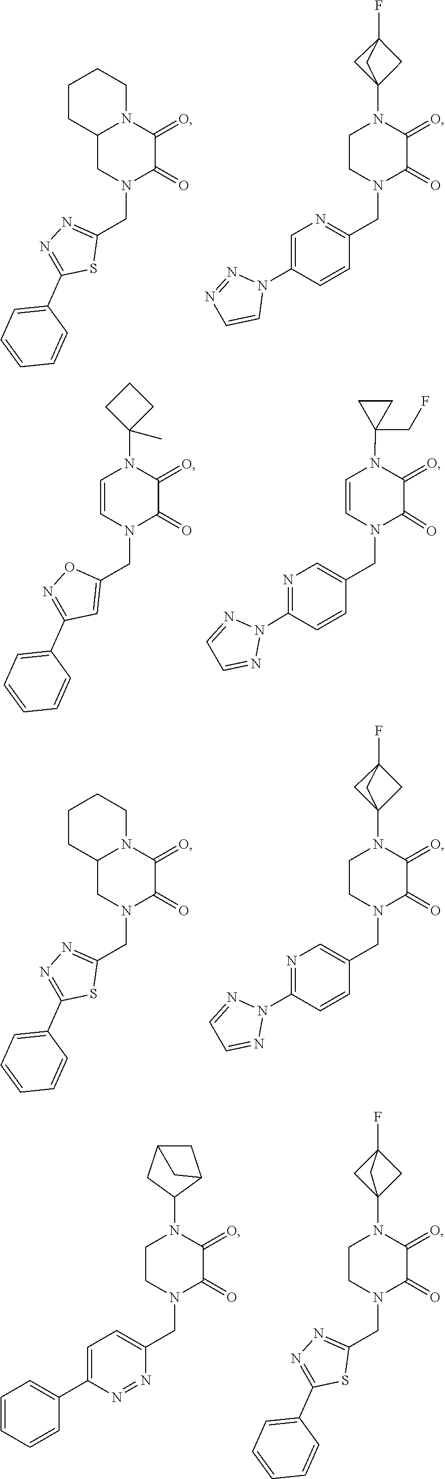

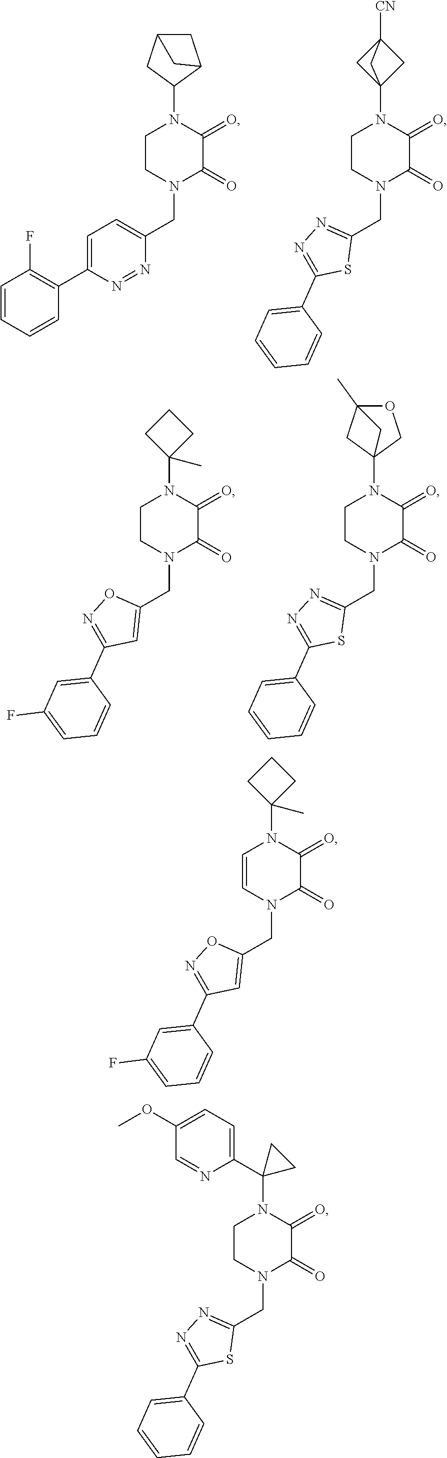

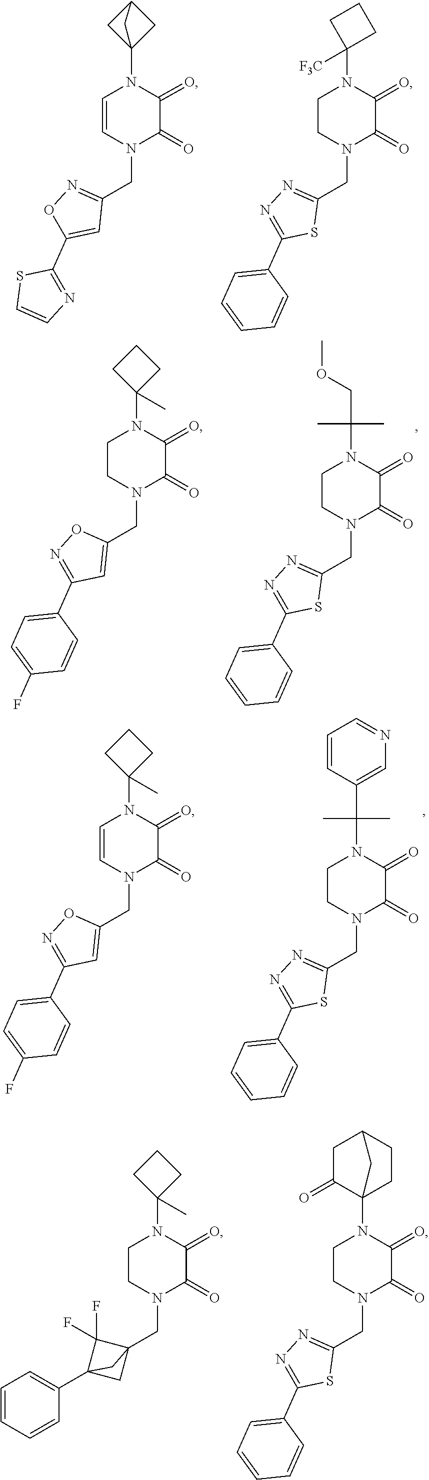

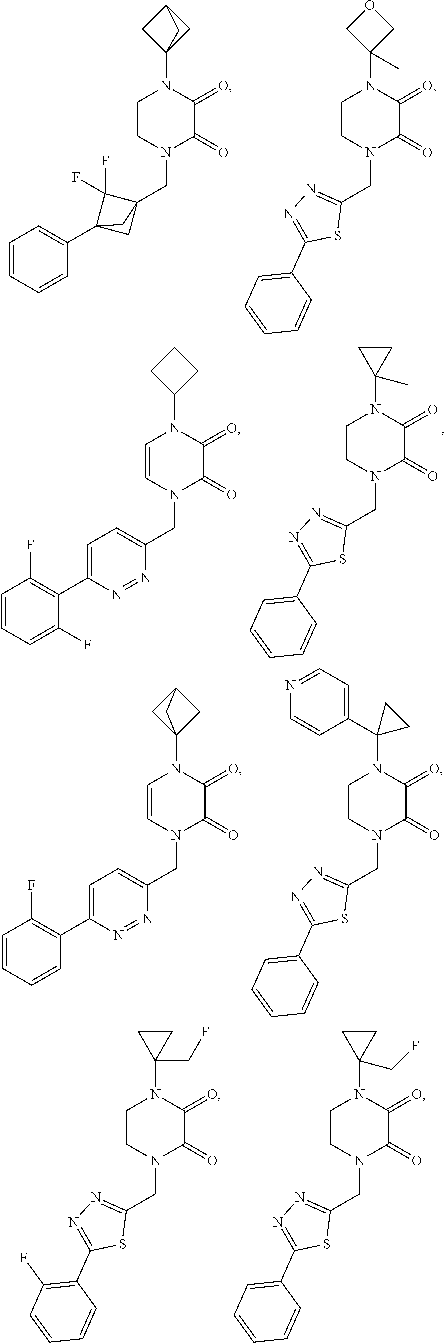

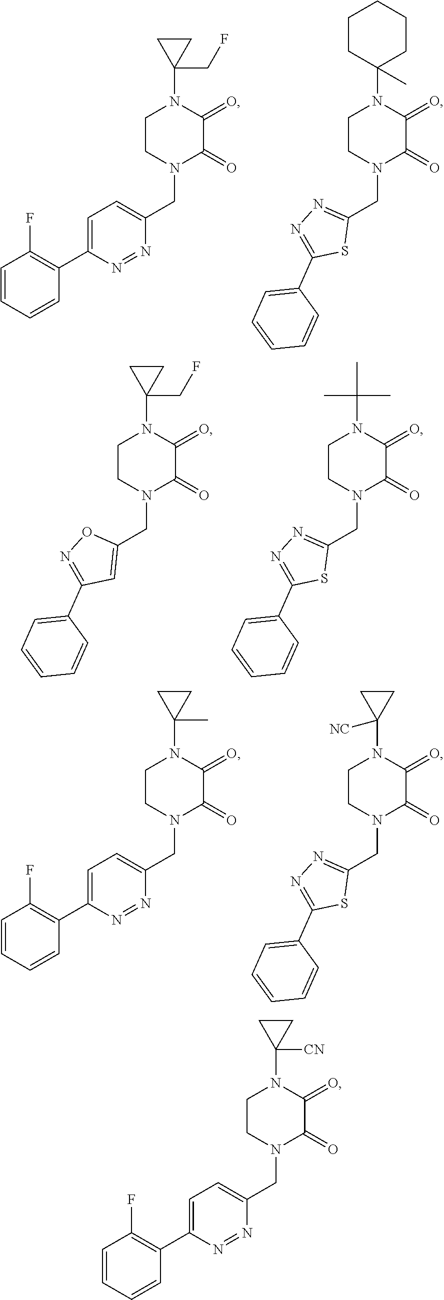

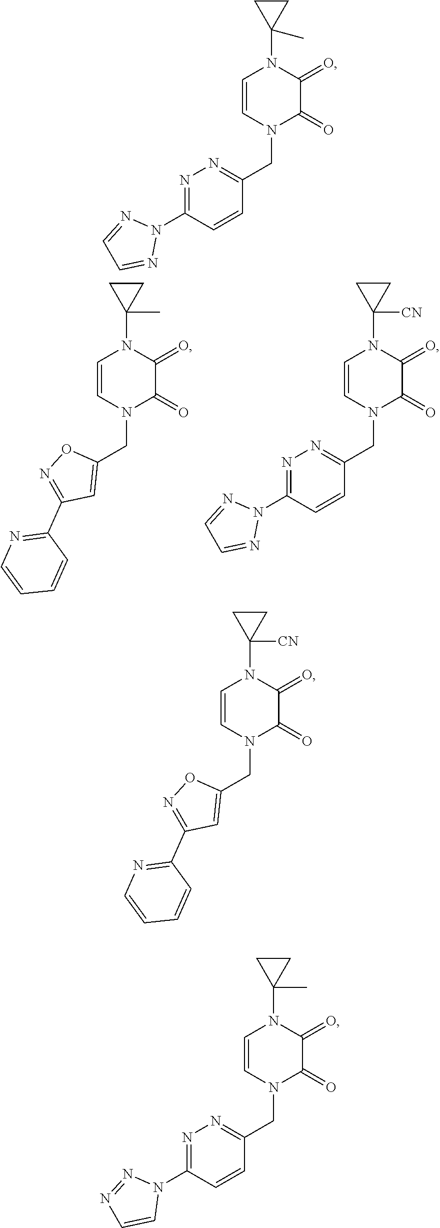

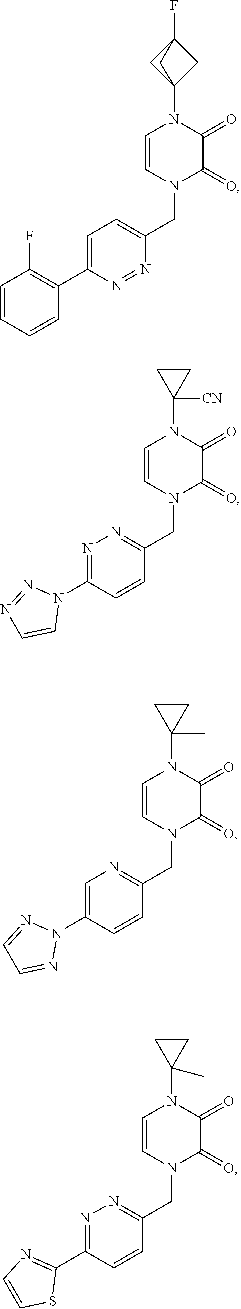 [SMILES: O=c1c(=O)n(C23CC(F)(C2)C3)ccn1Cc1ccc(-c2ccccc2F)nn1 ; N#CC1(n2ccn(Cc3ccc(-n4ccnn4)nn3)c(=O)c2=O)CC1 ; CC1(n2ccn(Cc3ccc(-n4nccn4)cn3)c(=O)c2=O)CC1 ; CC1(n2ccn(Cc3ccc(-c4nccs4)nn3)c(=O)c2=O)CC1]

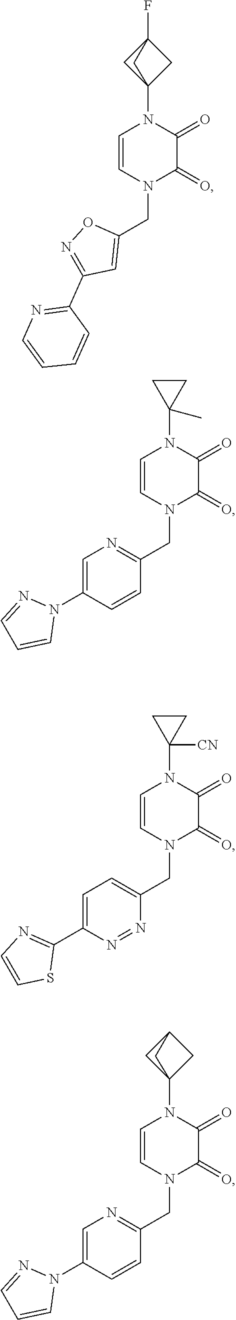 [SMILES: O=c1c(=O)n(C23CC(F)(C2)C3)ccn1Cc1cc(-c2ccccn2)no1 ; CC1(n2ccn(Cc3ccc(-n4cccn4)cn3)c(=O)c2=O)CC1 ; N#CC1(n2ccn(Cc3ccc(-c4nccs4)nn3)c(=O)c2=O)CC1 ; O=c1c(=O)n(C23CC(C2)C3)ccn1Cc1ccc(-n2cccn2)cn1]

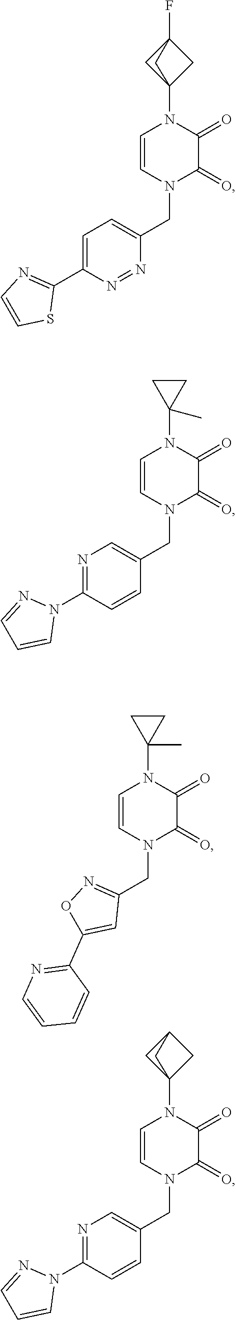 [SMILES: O=c1c(=O)n(C23CC(F)(C2)C3)ccn1Cc1ccc(-c2nccs2)nn1 ; CC1(n2ccn(Cc3ccc(-n4cccn4)nc3)c(=O)c2=O)CC1 ; CC1(n2ccn(Cc3cc(-c4ccccn4)on3)c(=O)c2=O)CC1 ; O=c1c(=O)n(C23CC(C2)C3)ccn1Cc1ccc(-n2cccn2)nc1]

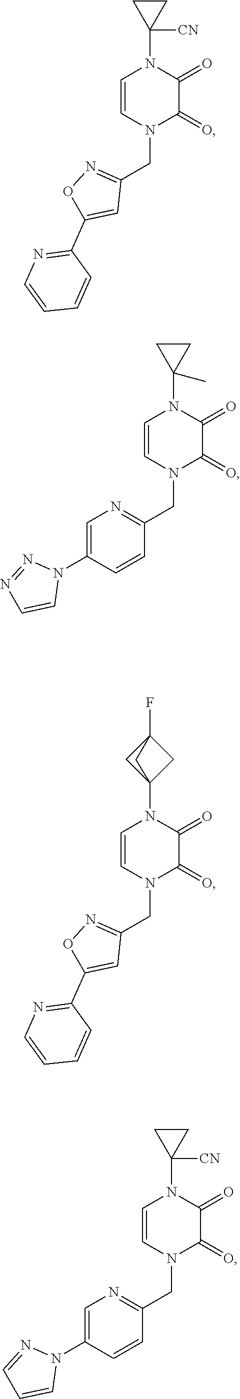 [SMILES: N#CC1(n2ccn(Cc3cc(-c4ccccn4)on3)c(=O)c2=O)CC1 ; CC1(n2ccn(Cc3ccc(-n4ccnn4)cn3)c(=O)c2=O)CC1 ; O=c1c(=O)n(C23CC(F)(C2)C3)ccn1Cc1cc(-c2ccccn2)on1 ; N#CC1(n2ccn(Cc3ccc(-n4cccn4)cn3)c(=O)c2=O)CC1]

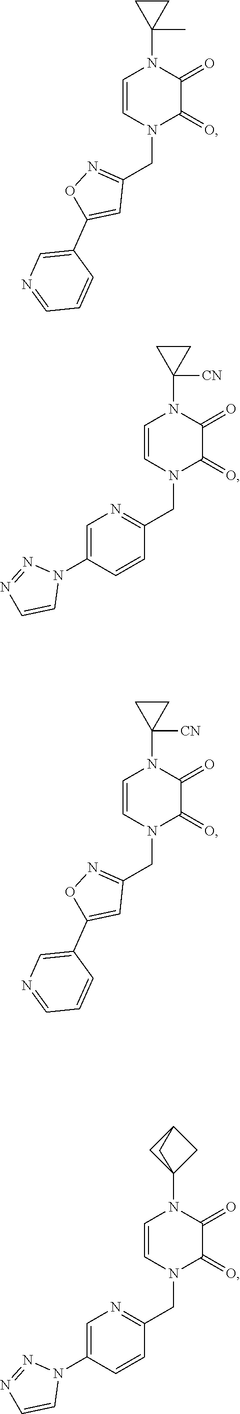 [SMILES: CC1(n2ccn(Cc3cc(-c4cccnc4)on3)c(=O)c2=O)CC1 ; N#CC1(n2ccn(Cc3ccc(-n4ccnn4)cn3)c(=O)c2=O)CC1 ; N#CC1(n2ccn(Cc3cc(-c4cccnc4)on3)c(=O)c2=O)CC1 ; O=c1c(=O)n(C23CC(C2)C3)ccn1Cc1ccc(-n2ccnn2)cn1]

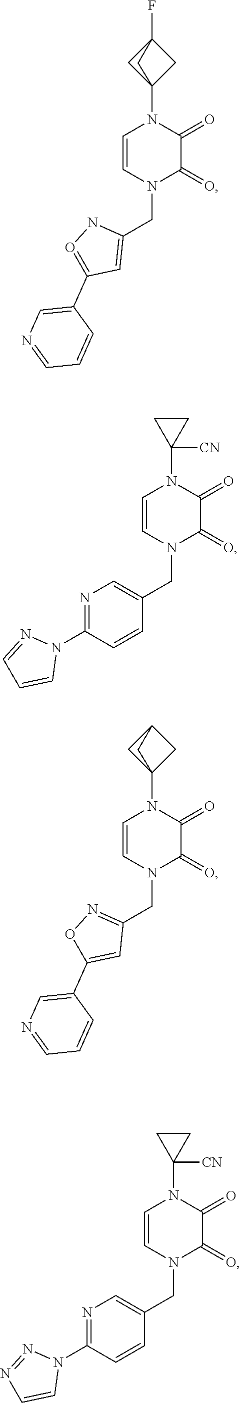

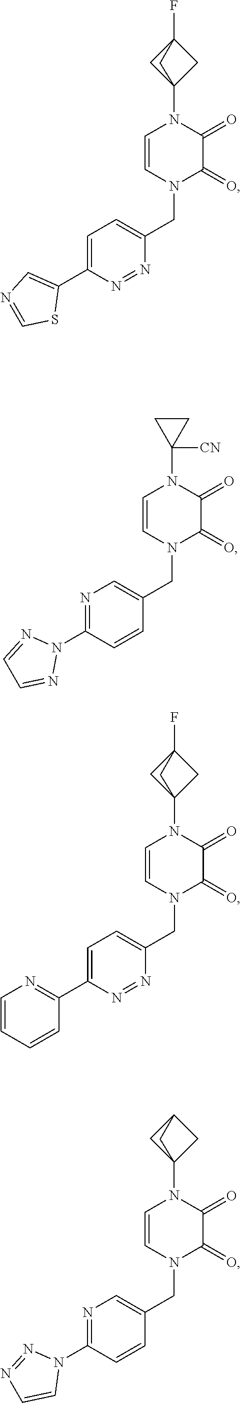 [SMILES: O=c1c(=O)n(C23CC(F)(C2)C3)ccn1Cc1ccc(-c2cncs2)nn1 ; N#CC1(n2ccn(Cc3ccc(-n4nccn4)nc3)c(=O)c2=O)CC1 ; O=c1c(=O)n(C23CC(F)(C2)C3)ccn1Cc1ccc(-c2ccccn2)nn1 ; O=c1c(=O)n(C23CC(C2)C3)ccn1Cc1ccc(-n2ccnn2)nc1]

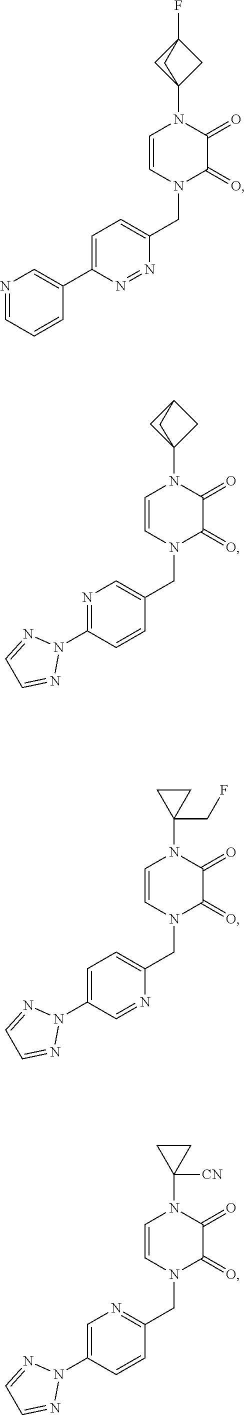 [SMILES: O=c1c(=O)n(C23CC(F)(C2)C3)ccn1Cc1ccc(-c2cccnc2)nn1 ; O=c1c(=O)n(C23CC(C2)C3)ccn1Cc1ccc(-n2nccn2)nc1 ; O=c1c(=O)n(C2(CF)CC2)ccn1Cc1ccc(-n2nccn2)cn1 ; N#CC1(n2ccn(Cc3ccc(-n4nccn4)cn3)c(=O)c2=O)CC1]

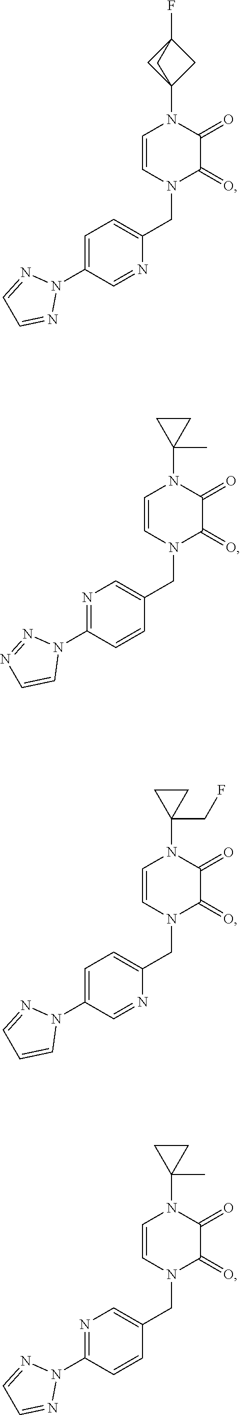 [SMILES: O=c1c(=O)n(C23CC(F)(C2)C3)ccn1Cc1ccc(-n2nccn2)cn1 ; CC1(n2ccn(Cc3ccc(-n4ccnn4)nc3)c(=O)c2=O)CC1 ; O=c1c(=O)n(C2(CF)CC2)ccn1Cc1ccc(-n2cccn2)cn1 ; CC1(n2ccn(Cc3ccc(-n4nccn4)nc3)c(=O)c2=O)CC1]

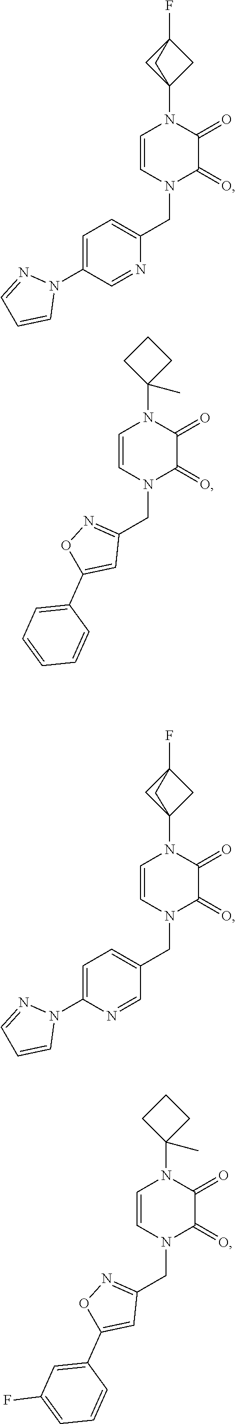 [SMILES: O=c1c(=O)n(C23CC(F)(C2)C3)ccn1Cc1ccc(-n2cccn2)cn1 ; CC1(n2ccn(Cc3cc(-c4ccccc4)on3)c(=O)c2=O)CCC1 ; O=c1c(=O)n(C23CC(F)(C2)C3)ccn1Cc1ccc(-n2cccn2)nc1 ; CC1(n2ccn(Cc3cc(-c4cccc(F)c4)on3)c(=O)c2=O)CCC1]

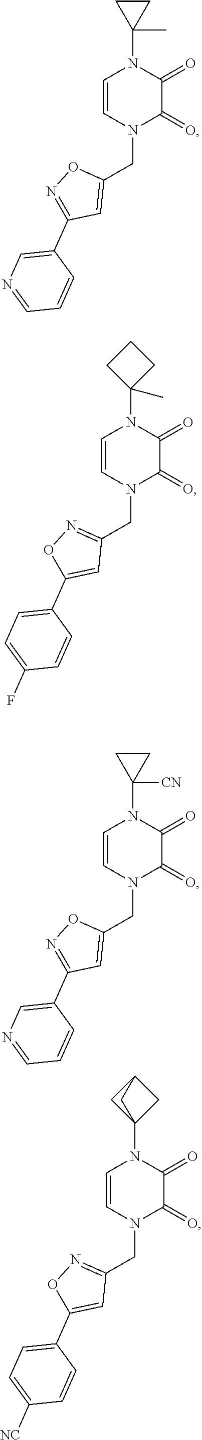 [SMILES: CC1(n2ccn(Cc3cc(-c4cccnc4)no3)c(=O)c2=O)CC1 ; CC1(n2ccn(Cc3cc(-c4ccc(F)cc4)on3)c(=O)c2=O)CCC1 ; N#CC1(n2ccn(Cc3cc(-c4cccnc4)no3)c(=O)c2=O)CC1 ; N#Cc1ccc(-c2cc(Cn3ccn(C45CC(C4)C5)c(=O)c3=O)no2)cc1]

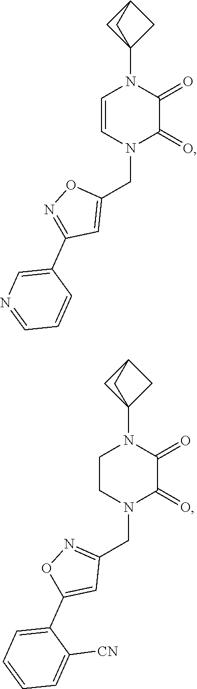 [SMILES: O=c1c(=O)n(C23CC(C2)C3)ccn1Cc1cc(-c2cccnc2)no1 ; N#Cc1ccccc1-c1cc(CN2CCN(C34CC(C3)C4)C(=O)C2=O)no1]

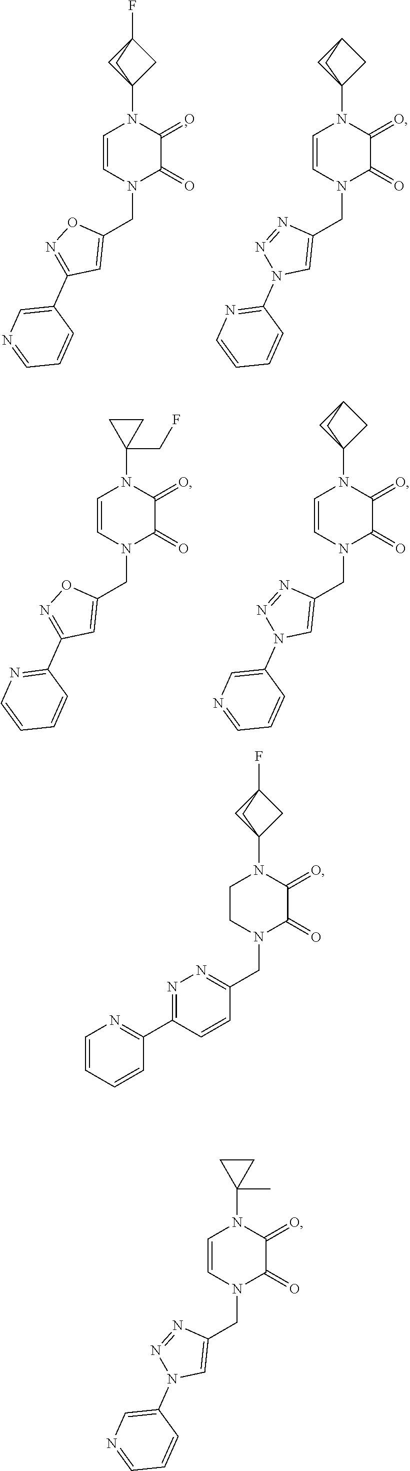

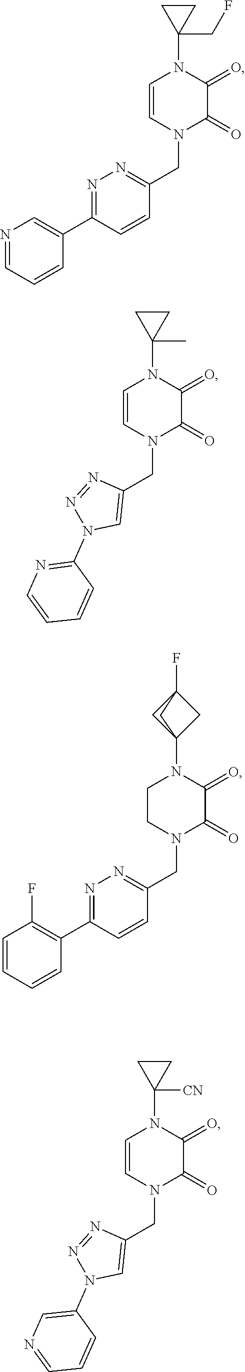 [SMILES: O=c1c(=O)n(C2(CF)CC2)ccn1Cc1ccc(-c2cccnc2)nn1 ; CC1(n2ccn(Cc3cn(-c4ccccn4)nn3)c(=O)c2=O)CC1 ; O=C1C(=O)N(C23CC(F)(C2)C3)CCN1Cc1ccc(-c2ccccc2F)nn1 ; N#CC1(n2ccn(Cc3cn(-c4cccnc4)nn3)c(=O)c2=O)CC1]

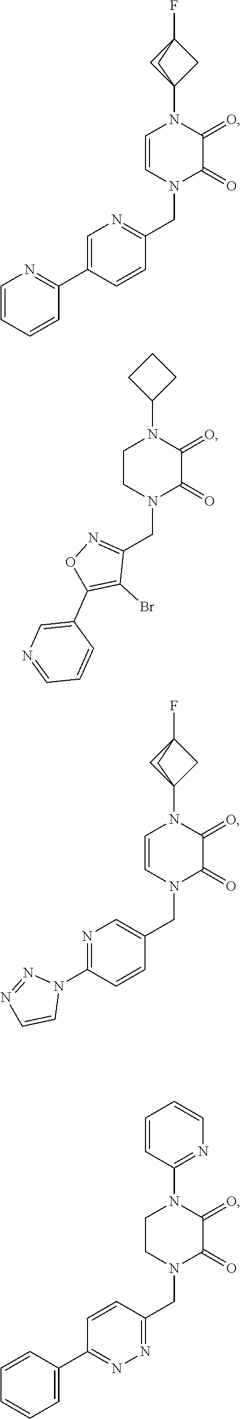 [SMILES: O=c1c(=O)n(C23CC(F)(C2)C3)ccn1Cc1ccc(-c2ccccn2)cn1 ; O=C1C(=O)N(C2CCC2)CCN1Cc1noc(-c2cccnc2)c1Br ; O=c1c(=O)n(C23CC(F)(C2)C3)ccn1Cc1ccc(-n2ccnn2)nc1 ; O=C1C(=O)N(c2ccccn2)CCN1Cc1ccc(-c2ccccc2)nn1]

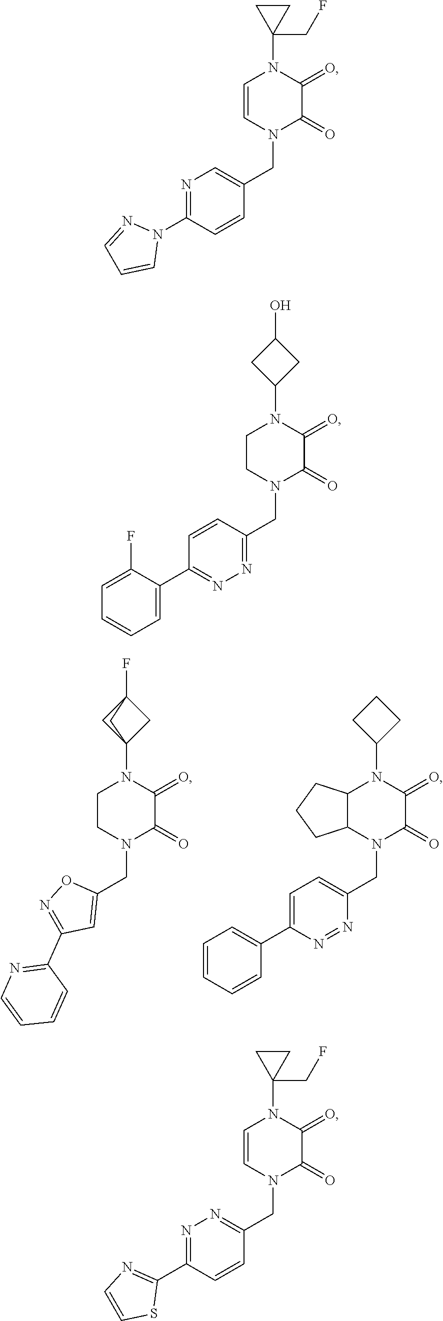

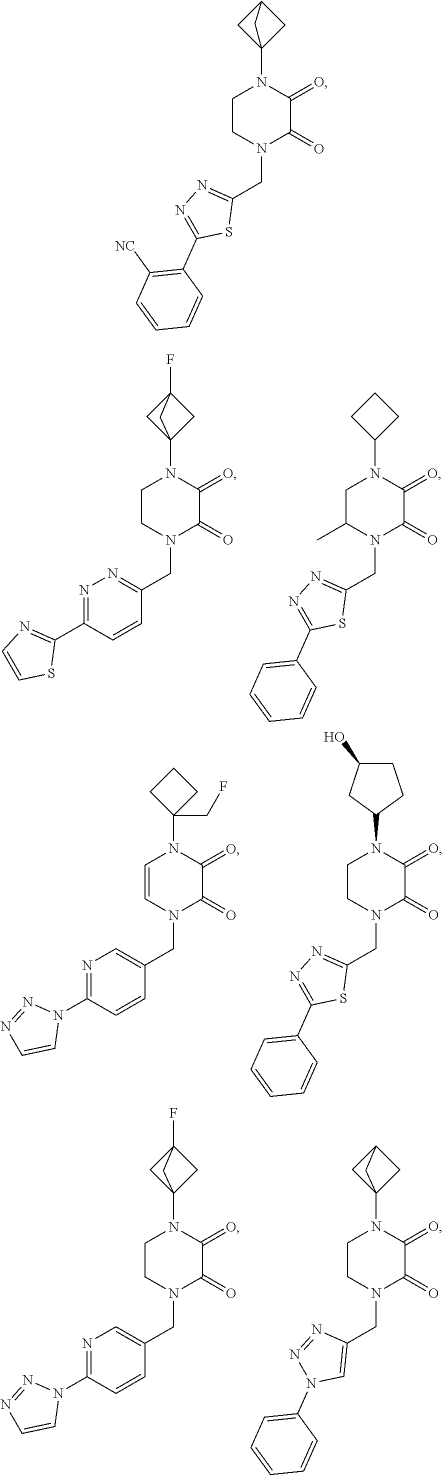

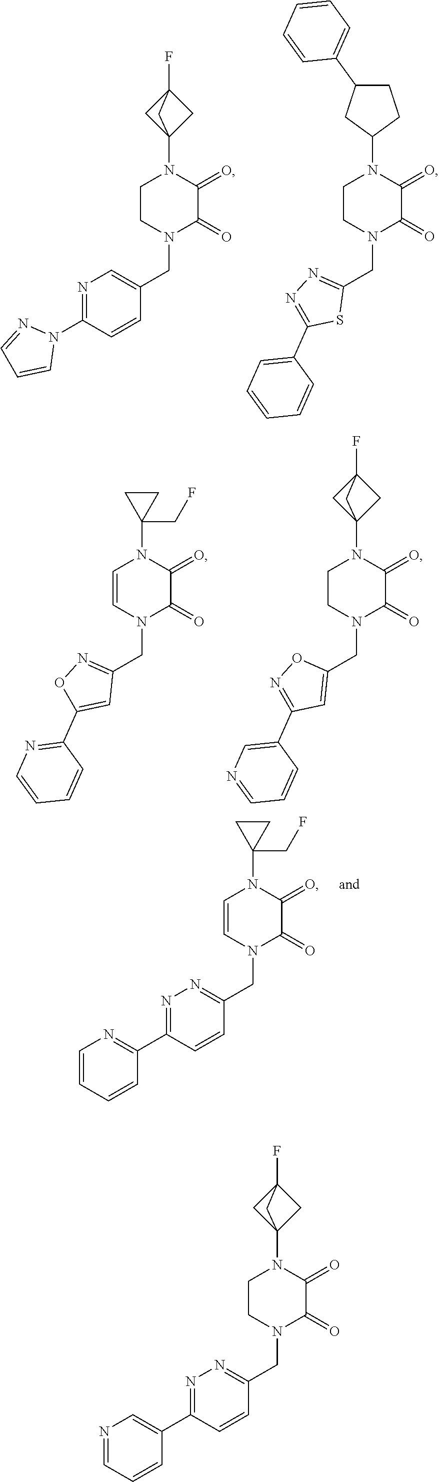

or a pharmaceutically acceptable salt thereof.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “-O-alkyl,” etc. . . .

As used herein “H” and hydrogen are used interchangeably.

The term “alkylene,” or “alkylenyl” by itself or as part of another substituent means a divalent straight or branched chain hydrocarbon radical having the stated number of carbon atoms. For example, —(C₁-C₅) alkylenyl, would include, e.g., —CH₂—, —CH(CH₃)—, —CH₂—CH(CH₃)—, —CH(CH₃)—CH₂—, —CH₂CH₂—, —CH₂CH₂CH₂—, —CH₂CH₂CH₂CH₂—, —CH₂CH(CH₃)CH₂— or —CH₂CH₂CH₂CH₂CH₂—.

The term “halogen” includes fluorine, chlorine, bromine and iodine.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “C₃-C₆cycloalkyl” encompasses bridged, saturated or unsaturated cycloalkyl groups having 3 to 6 carbons. Examples of cycloalkyl include cyclopropyl, cyclobutyl, cyclopentyl and cyclohexyl or bicyclo[1.1.1pentanyl, bicyclo[3.1.0]hexanyl.

The term “C₃-C₁₀cycloalkyl” encompasses bridged, saturated or unsaturated cycloalkyl groups having 3 to 10 carbons. “Cycloalkyl” also includes non-aromatic rings and monocyclic, non-aromatic rings fused to a saturated cycloalkyl group and aromatic rings fused to a saturated cycloalkyl group. Examples of cycloalkyl include cyclopropyl, cyclobutyl, cyclopentyl, cyclohexyl, cycloheptyl, tetrahydronaphthyl, decahydronaphthyl, indanyl and the like. Examples described by structure include:

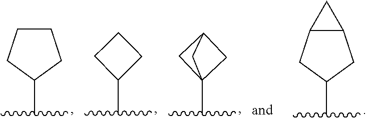

The term “halogen-substituted phenyl” or phenyl substituted with one or more halogens refers to a phenyl ring with the hydrogen atoms thereof being partially or completely substituted with halogen, examples thereof including fluorophenyl, difluorophenyl, or trifluorophenthyl.

The term “heteroaryl” means an aromatic ring that contains at least one ring heteroatom selected from O, S and N. Examples of heteroaryl groups include pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, isoquinolyl, and the like.

The term “heterocycloalkyl” means mono- or bicyclic or bridged partially unsaturated or saturated rings containing at least one heteroatom selected from N, S and O, each of said rings having from 3 to 10 atoms in which the point of attachment may be carbon or nitrogen. Examples include tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and tetrahydropyran. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. Examples described by structure include:

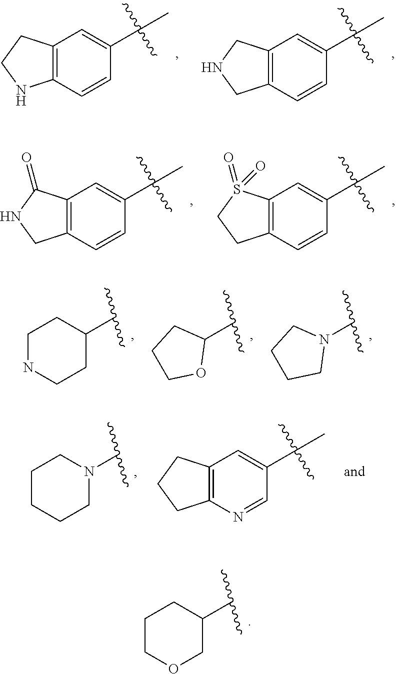

The term “nitrogen-containing ring” means mono- or bicyclic or bridged saturated, unsaturated or aromatic ring containing at least one nitrogen, possible 1, 2, 3 or 4 nitrogens, each of said ring having from 5 to 11 atoms in which the point of attachment may be carbon or nitrogen. Additionally, the nitrogen-containing ring can contain other heteroatoms such as O or S.

The term “alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidinyl, glucamine, glucosamine, histidine, hydrabamine, isopropylamine, lysine, methylglucamine, morpholine, piperazine, piperidinyl, polyamine resins, procaine, purines, theobromine, triethylamine, trimethylamine, tripropylamine, tromethamine, and the like.

The term “patient” refers to a mammalian patient, including a human, canine, feline, bovine, or porcine patient, preferably a human patient, receiving or about to receive medical treatment. A patient “in need of treatment” is an individual diagnosed with, suspected of having, or predisposed to a disease or disorder in which a compound or composition of the invention is intended to treat or ameliorate (e.g. an IL4I1-related diseases such as cancer), or a patient for whom prevention of such a disorder is desired.

“Treat” or “treatment” means to administer an agent, such as a composition containing any of the compounds described herein, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of an agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

The compounds of the present invention may contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

Some of the compounds described herein contain substituted cycloalkanes having cis- and trans-isomers, and unless specified otherwise, are meant to include both cis- and trans-geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that the present invention is meant to include the pharmaceutically acceptable salts, and also salts that are not pharmaceutically acceptable, of the compounds described herein, when they are used as precursors to the free compounds or their pharmaceutically acceptable salts or in other synthetic manipulations.

Solvates, and in particular, the hydrates of the compounds of the structural formulas described herein are included in the present invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically enriched compounds can be prepared without undue experimentation by conventional techniques well known to those skilled in the art or by processes analogous to those described in the Schemes and Examples herein using appropriate isotopically enriched reagents or Intermediates.

It should be noted that chemically unstable compounds are excluded from the embodiments contained herein.

### Methods of Treatment

Also encompassed by the present invention are methods of preventing, treating or ameliorating IL4I1-related diseases. The compounds described herein can be effective in preventing, treating or ameliorating various IL4I1-related diseases, such as cancer. Described herein are methods for treatment of cancer displaying IL4I1-expressing cells in a patient comprising administration of a compound of the invention, or a pharmaceutical salt thereof, to the patient. Described herein are methods for prevention of cancer displaying IL4I1-expressing cells in a patient comprising administration of a compound of the invention, or a pharmaceutical salt thereof, to the patient. Described herein are methods for ameliorating the symptoms or clinical effects of cancer displaying IL4I1-expressing cells in a patient comprising administration of a compound of the invention, or a pharmaceutical salt thereof, to the patient.

In one embodiment described herein, the cancer to be treated is selected from the group consisting of cancers displaying IL4I1-expressing cells and lymphomas displaying IL4I1-expressing cells. In certain embodiment, the cancers to be treated are solid tumors. In certain embodiments, the cancers to be treated are selected from carcinomas, sarcomas, mesotheliomas, blastomas and germ cell tumors. In another particular embodiment, the cancer to be treated is a cancer displaying IL4I1-expressing cells selected from the group consisting of mesotheliomas, non-small-cell lung carcinomas, colon carcinoma, breast carcinoma, thyroid carcinoma, testicular germ cell tumors and ovarian carcinoma.

In another specific embodiment, the cancer to be treated is a lymphoma displaying IL4I1-expressing cells such as a B-cell lymphoma displaying IL4I1-expressing cells.

In certain embodiments, the cancer to be treated is a cancer displaying IL4I1-expressing cells selected from the group consisting of PMBL (Primary Mediastinal large B-cell Lymphoma), classical Hodgkin lymphomas (cHL), NLPHL (Nodular lymphocyte predominant Hodgkin's lymphoma), non-mediastinal Diffuse Large B-Cell Lymphoma (DLBCL) and SLL/CLL (Small Lymphocytic Lymphoma/Chronic Lymphocytic Leukemia). In another specific embodiment, the cancer to be treated is a lymphoma displaying IL4I1-expressing cells.

In one embodiment described herein, the cancer to be prevented is selected from the group consisting of cancers displaying IL4I1-expressing cells and lymphomas displaying IL4I1-expressing cells. In a certain embodiment, the cancer to be prevented is a solid tumor. In certain embodiments, the cancer to be prevented is selected from carcinomas, sarcomas, mesotheliomas, blastomas and germ cell tumors. In another particular embodiment, the cancer to be prevented is a cancer displaying IL4I1-expressing cells selected from the group consisting of mesotheliomas, non-small-cell lung carcinomas, colon carcinoma, breast carcinoma, thyroid carcinoma, testicular germ cell tumors and ovarian carcinoma.

In another specific embodiment, the cancer to be prevented is a lymphoma displaying IL4I1-expressing cells such as a B-cell lymphoma displaying IL4I1-expressing cells.

In certain embodiments, the cancer to be prevented is a cancer displaying IL4I1-expressing cells selected from the group consisting of PMBL, cHL, NLPHL, DLBCL and SLL/CLL. In another specific embodiment, the cancer to be treated is a lymphoma displaying IL4I1-expressing cells.

In one embodiment described herein, the cancer to be ameliorated is selected from the group consisting of cancers displaying IL4I1-expressing cells and lymphomas displaying IL4I1-expressing cells. In a certain embodiment, the cancer to be ameliorated is a solid tumor. In certain embodiments, the cancer to be ameliorated is selected from carcinomas, sarcomas, mesotheliomas, blastomas and germ cell tumors. In another particular embodiment, the cancer to be ameliorated is a cancer displaying IL4I1-expressing cells selected from the group consisting of mesotheliomas, non-small-cell lung carcinomas, colon carcinoma, breast carcinoma, thyroid carcinoma, testicular germ cell tumors and ovarian carcinoma.

In another specific embodiment, the cancer to be ameliorated is a lymphoma displaying IL4I1-expressing cells such as a B-cell lymphoma displaying IL4I1-expressing cells.

In certain embodiments, the cancer to be ameliorated is a cancer displaying IL4I1-expressing cells selected from the group consisting of PMBL, cHL, NLPHL, DLBCL and SLL/CLL. In another specific embodiment, the cancer to be treated is a lymphoma displaying IL4I1-expressing cells.

### Pharmaceutical Compositions

Compounds described herein may be administered orally or parenterally. As formulated into a dosage form suitable for administration, the compounds described herein can be used as a pharmaceutical composition for the prevention, treatment, or remedy of the above diseases.

Accordingly, the invention relates to pharmaceutical compositions comprising a therapeutically effective amount of a compound of the invention (i.e. a compound of Formula I or II, or any IL4i1 inhibitor compound described herein), or a pharmaceutically acceptable salt thereof, and a pharmaceutically acceptable carrier. As used herein, a “therapeutically effective amount” is an amount sufficient to product the desired clinical outcome, e.g. treatment or prevention of cancer displaying Il4i1-expressing cells or amelioration of the clinical effects or presentation thereof. Such a therapeutically effective amount may be contained in a single dosage form (e.g. one tablet or injection) or split into more than one of the dosage form (e.g. more than one tablet or injection, which together contain a therapeutically effective amount).

In clinical use of the compounds described herein, usually, the compound is formulated into various preparations together with pharmaceutically acceptable additives according to the dosage form and may then be administered. By “pharmaceutically acceptable” it is meant the additive, carrier, diluent or excipient must be compatible with the other ingredients of the formulation and not deleterious to the recipient thereof. As such, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropyl cellulose, sorbitol, sorbitan fatty acid ester, polysorbate, sucrose fatty acid ester, polyoxymethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders and suppositories; and liquid preparations such as syrups, elixirs and injections. These may be formulated according to conventional methods known in the field of pharmaceutical preparations. The liquid preparations may also be in such a form that may be dissolved or suspended in water or in any other suitable medium in their use. Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9% by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. In specific embodiments, the dose is from about 0.01 to about 25 mg/kg/day, in particular embodiments, from about 0.05 to about 10 mg/kg/day, or from about 0.001 to about 50 mg/kg/day. For oral administration, the compositions are preferably provided in the form of tablets or capsules containing from 0.01 mg to 1,000 mg. In specific embodiments, the dose is 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 or 1,000 milligrams of a compound described herein. This dosage regimen may be adjusted to provide the optimal therapeutic response.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds described herein or the other drugs may have utility, where the combination of the drugs together are safer or more effective than either drug alone. Such other drug(s) may be administered in an amount commonly used therefore, contemporaneously or sequentially with a compound described herein or a pharmaceutically acceptable salt thereof. When a compound described herein is used contemporaneously with one or more other drugs, the pharmaceutical composition may in specific embodiments contain such other drugs and the compound described herein or its pharmaceutically acceptable salt in unit dosage form. However, the combination therapy may also include therapies in which the compound described herein or its pharmaceutically acceptable salt and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound described herein or a pharmaceutically acceptable salt thereof.

Examples of other active ingredients that may be administered in combination with a compound of any of the Formulas described herein (e.g. Formula I and II) or a pharmaceutically acceptable salt thereof and either administered separately or in the same pharmaceutical composition, include, but are not limited to pain relieving agents, anti-angiogenic agents, anti-neoplastic agents, anti-diabetic agents, anti-infective agents, or gastrointestinal agents, or combinations thereof.

Suitable compounds that may be used in combination with a compound according to the present invention include without limitation sildenafil, vardenafil, tadalafil and alprostadil, epoprostenol, iloprost, bosentan, amlodipine, diltiazem, nifedipine, ambrisentan and warfarin, fluticasone, budesonide, mometasone, flunisolide, beclomethasone, montelukast, zafirlukast, zileuton, salmeterol, formoterol, theophylline, albuterol, levalbuterol, pirbuterol, ipratropium, prednisone, methylprednisolone, omalizumab, corticosteroid and cromolyn, atorvastatin, lovastatin, simvastatin, pravastatin, fluvastatin, rosuvastatin, gemfibrozil, fenofibrate, nicotinic acid, and clopidogrel and pharmaceutically acceptable salts thereof.

Additionally, a compound of any of the Formulas disclosed herein (e.g. Formula I and II) may be used in combination with one or more other active agents, including but not limited to, other anti-cancer agents that are used in the prevention, treatment, control, amelioration, or reduction of risk of a particular disease or condition (e.g., cell proliferation disorders). In one embodiment, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present invention.

In one embodiment, the other active agent is selected from the group consisting of vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines, CTLA-4, LAG-3 and PD-1 antagonists.

PD-1 is recognized as having an important role in immune regulation and the maintenance of peripheral tolerance. PD-1 is moderately expressed on naive T-cells, B-cells and NKT-cells and up-regulated by T-cell and B-cell receptor signaling on lymphocytes, monocytes and myeloid cells (Sharpe et al., Nature Immunology (2007); 8:239-245).

Two known ligands for PD-1, PD-L1 (B7-H1) and PD-L2 (B7-DC) are expressed in human cancers arising in various tissues. In large sample sets of, for example, ovarian, renal, colorectal, pancreatic, and liver cancers, and in melanoma, it was shown that PD-L1 expression correlated with poor prognosis and reduced overall survival irrespective of subsequent treatment. (Dong et al., Nat Med. 8(8):793-800 (2002); Yang et al., Invest Ophthamol Vis Sci. 49: 2518-2525 (2008); Ghebeh et al., Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953; Inman et al., Cancer 109: 1499-1505 (2007); Shimauchi et al., Int. J. Cancer 121:2585-2590 (2007); Gao et al., Clin. Cancer Research 15: 971-979 (2009); Nakanishi J., Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T-cells in breast cancer and melanoma (Ghebeh et al., BMC Cancer. 2008 8:5714-15 (2008); and Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T-cells to attenuate T-cell activation and to evade immune surveillance, thereby contributing to an impaired immune response against the tumor.

Immune checkpoint therapies targeting the PD-1 axis have resulted in groundbreaking improvements in clinical response in multiple human cancers (Brahmer, et al., N Engl J Med 2012, 366: 2455-65; Garon et al., N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; and Wolchok et al., N Engl J Med 2013, 369: 122-33).

“PD-1 antagonist” means any chemical compound or biological molecule that blocks binding of PD-L1 expressed on a cancer cell to PD-1 expressed on an immune cell (T-cell, B-cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP_054862 and NP_079515, respectively.

PD-1 antagonists useful in any of the treatment methods, medicaments and uses of the present invention include a monoclonal antibody (mAb), or antigen binding fragment thereof, which specifically binds to PD-1 or PD-L1, and preferably specifically binds to human PD-1 or human PD-Ll. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments. Examples of PD-1 antagonists include, but are not limited to, pembrolizumab (KEYTRUDA®, Merck and Co., Inc., Kenilworth, NJ, USA). “Pembrolizumab” (formerly known as MK-3475, SCH 900475 and lambrolizumab and sometimes referred to as “pembro”) is a humanized IgG4 mAb with the structure described in WHO Drug Information, Vol. 27, No. 2, pages 161-162 (2013). Additional examples of PD-1 antagonists include nivolumab (OPDIVO®, Bristol-Myers Squibb Company, Princeton, NJ, USA), atezolizumab (MPDL3280A; TECENTRIQ®, Genentech, San Francisco, CA, USA), durvalumab (IMFINZI®, Astra Zeneca Pharmaceuticals, LP, Wilmington, DE, avelumab (BAVENCIO®, Merck KGaA, Darmstadt, Germany and Pfizer, Inc., New York, NY), cemiplimab (LIBTAYO®, Regeneron Pharmaceuticals, Inc., Tarrytown, NY, and Sanofi-Aventis LLC, Bridgewater, NJ), dostarlimab (JEMPERLI®, GlaxoSmithKline LLC, Philadelphia, PA), Sasanlimab (PF-06801591), Retifanlimab (MGA012), Cetrelimab (JNJ-63723283), Tebotelimab (MGD013), Cadonilimab, (AK104), Ezabenlimab (BI754091), Budigalimab (ABBV-181), Spartalizumab (PDR001), Zimberelimab (AB122), Serplulimab (HLX10), Cosibelimab (CK-301), Sugemalimab (CS1001), Camrelizumab, Sintilimab, Tislelizumab, Toripalimab (TAB001), Penpulimab (AK105) and Adebrelimab (SHR-1316).

Examples of monoclonal antibodies (mAbs) that bind to human PD-1, which are useful in the treatment methods, medicaments and uses of the present invention, are described in U.S. Pat. Nos. 7,488,802, 7,521,051, 8,008,449, 8,354,509, 8,168,757, WO2004/004771, WO2004/072286, WO2004/056875, and US2011/0271358.

Examples of mAbs that bind to human PD-L1, which are useful in the treatment methods, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in any of the treatment methods, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010/027827 and WO2011/066342. Specific fusion proteins useful as the PD-1 antagonist in the treatment methods, medicaments and uses of the present invention include AMP-224 (also known as B7-DCIg), which is a PD-L2-FC fusion protein that binds to human PD-1.

Thus, one embodiment provides a method of treating cancer comprising administering an effective amount of a compound of the invention, or a pharmaceutically acceptable salt thereof, in combination with a PD-1 antagonist to a subject in need thereof. In such embodiments, a compound of the invention, or a pharmaceutically acceptable salt thereof, and a PD-1 antagonist are administered concurrently or sequentially.

Specific non-limiting examples of such cancers which can be treated in accordance with this embodiment include melanoma (including unresectable or metastatic melanoma), head & neck cancer (including recurrent or metastatic head and neck squamous cell cancer), classical Hodgkin lymphoma, urothelial carcinoma, gastric cancer, cervical cancer, primary mediastinal large-B-cell lymphoma, microsatellite instability-high cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, and salivary cancer.

In one embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from unresectable or metastatic melanoma, recurrent or metastatic head and neck squamous cell cancer, classical Hodgkin lymphoma, urothelial carcinoma, gastric cancer, cervical cancer, primary mediastinal large-B-cell lymphoma, microsatellite instability-high or mismatch repair deficient cancer, esophageal cancer, renal cancer, endometrial carcinoma, tumor mutational burden-high cancer, triple negative breast cancer, non-small cell lung cancer, and hepatocellular carcinoma. In one such embodiment, the agent is a PD-1 antagonist. In one such embodiment, the agent is selected from the group consisting of pembrolizumab, nivolumab, atezolizumab, durvalumab, avelumab, cemiplimab, and dostarlimab, or any of the anti-PD-1 and anti-PD-L1 antibodies disclosed herein. In one such embodiment, the agent is pembrolizumab. In another such embodiment, the agent is nivolumab. In another such embodiment, the agent is atezolizumab.

Pembrolizumab is approved by the U.S. FDA for the treatment of patients with unresectable or metastatic melanoma, Stage IIB, IIC, or III melanoma following complete resection, non-small cell lung cancer (NSCLC), head and neck squamous cell cancer (HNSCC), classical Hodgkin Lymphoma (cHL), microsatellite instability-high or mismatch repair deficient cancer, microsatellite instability-high or mismatch repair deficient colorectal cancer (CRC), primary mediastinal large B-cell lymphoma, gastric cancer, urothelial cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, triple-negative breast cancer (TNBC), as described in the Prescribing Information for KEYTRUDA™ (Merck & Co., Inc., Whitehouse Station, NJ USA; initial U.S. approval 2014, updated February 2022). In another embodiment, there is provided a method of treating cancer comprising administering an effective amount of a compound of the invention, or a pharmaceutically acceptable salt thereof, to a person in need thereof, in combination with pembrolizumab, wherein said cancer is selected from unresectable or metastatic melanoma, Stage IIB, IIC, or III melanoma following complete resection, non-small cell lung cancer (NSCLC), head and neck squamous cell cancer (HNSCC), classical Hodgkin Lymphoma (cHL), microsatellite instability-high or mismatch repair deficient cancer, microsatellite instability-high or mismatch repair deficient colorectal cancer (CRC), gastric cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, cutaneous squamous cell carcinoma (cSCC), triple-negative breast cancer (TNBC).

In another embodiment, there is provided a method of treating cancer comprising administering an effective amount of a compound of the invention, or a pharmaceutically acceptable salt thereof in combination with a PD-1 antagonist, to a person in need thereof, wherein said cancer is selected from unresectable or metastatic melanoma, Stage IIB, IIC, or III melanoma following complete resection, non-small cell lung cancer, head and neck squamous cell cancer, classical Hodgkin Lymphoma, microsatellite instability-high or mismatch repair deficient cancer, microsatellite instability-high or mismatch repair deficient colorectal cancer, gastric cancer, esophageal cancer, cervical cancer, hepatocellular carcinoma, Merkel cell carcinoma, renal cell carcinoma, endometrial carcinoma, tumor mutational burden-high cancer, cutaneous squamous cell carcinoma, triple-negative breast cancer. In one such embodiment, the agent is a PD-1 antagonist. In one such embodiment, the agent is selected from the group consisting of pembrolizumab, nivolumab, atezolizumab, durvalumab, avelumab, cemiplimab, and dostarlimab, or any of the anti-PD-1 and anti-PD-L1 antibodies disclosed herein. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab. In another such embodiment, the agent is cemiplimab. In another such embodiment, the agent is dostarlimab.

In another embodiment, there is provided a method of treating cancer comprising administering an effective amount of a compound of the invention, or a pharmaceutically acceptable salt thereof, in combination with a PD-1 antagonist, to a person in need thereof, wherein said cancer is selected from melanoma, non-small cell lung cancer, small cell lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer. In one such embodiment, the agent is a PD-1 antagonist. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab. In another such embodiment, the agent is cemiplimab. In another such embodiment, the agent is dostarlimab.

In one embodiment, there is provided a method of treating unresectable or metastatic melanoma comprising administering an effective amount of a compound of the invention, or a pharmaceutically acceptable salt thereof, in combination with a PD-1 antagonist to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating recurrent or metastatic head and neck squamous cell cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating classical Hodgkin lymphoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating triple-negative breast cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating urothelial carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating gastric cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating cervical cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating primary mediastinal large-B-cell lymphoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating microsatellite instability-high (MSI-H) cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating non-small cell lung cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating hepatocellular carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating Merkel cell carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating renal cell carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating endometrial cell carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating cutaneous squamous cell carcinoma comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating tumor mutational burden-high cancer comprising administering an effective amount of a compound of the invention, or a pharmaceutically acceptable salt thereof, in combination with a PD-1 antagonist, to a person in need thereof. In one such embodiment, the agent is selected from the group consisting of pembrolizumab, nivolumab, atezolizumab, durvalumab, avelumab, cemiplimab, and dostarlimab. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

Examples of vascular endothelial growth factor (VEGF) receptor inhibitors include, but are not limited to, bevacizumab (sold under the trademark AVASTIN by Genentech/Roche), axitinib, (N-methyl-2-[[3-[([pound])-2-pyridin-2-ylethenyl]-1H-indazol-6-yl]sulfanyl]benzamide, also known as AG013736, and described in PCT Publication No. WO01/002369), Brivanib Alaninate ((S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indoi-6-yl)-2-[(4-pyridinyimethy)amino]-3-pyridinecarboxamide. and described in PCT Publication No. WO 02/068470), pasireotide (also known as SO 230, and described in PCT Publication No. WO02/010192), and sorafenib (sold under the tradename NEXAVAR).

Examples of topoisomerase II inhibitors include but are not limited to, etoposide (also known as VP-16 and Etoposide phosphate, sold under the tradenames TOPOSAR, VEPESID and ETOPOPHOS), and teniposide (also known as VM-26, sold under the tradename VUMON).

Examples of alkylating agents include but are not limited to, 5-azacytidine (sold under the trade name VIDAZA), decitabine (sold under the trade name of DECOGEN), temozolomide (sold under the trade names TEMODAR and TEMODAL by Merck & Co., Inc., Kenilworth, NJ, USA), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (sold under the tradename BCNU), bendamustine (sold under the tradename TREANDA), busulfan (sold under the tradenames BUSULFEX and MYLERAN), carboplatin (sold under the tradename PARAPLATIN), lomustine (also known as CCNU, sold under the tradename CeeNU), cisplatin (also known as CDDP, sold under the tradenames PLATINOL and PLATINOL-AQ), chlorambucil (sold under the tradename LEUKERAN), cyclophosphamide (sold under the tradenames CYTOXAN and NEOSAR), dacarbazine (also known as DTIC, DIC and imidazole carboxamide, sold under the tradename DTIC-DOME), altretamine (also known as hexamethylmelamine (HMM) sold under the tradename HEXALEN), ifosfamide (sold under the tradename IFEX), procarbazine (sold under the tradename MATULANE), mechlorethamine (also known as nitrogen mustard, mustine and mechloroethamine hydrochloride, sold under the tradename MUSTARGEN), streptozocin (sold under the tradename ZANOSAR), thiotepa (also known as thiophosphoamide, TESPA and TSPA, and sold under the tradename THIOPLEX).

Examples of anti-tumor antibiotics include, but are not limited to, doxorubicin (sold under the tradenames ADRIAMYCIN and RUB EX), bleomycin (sold under the tradename LENOXANE), daunorubicin (also known as dauorubicin hydrochloride, daunomycin, and rubidomycin hydrochloride, sold under the tradename CERUBIDINE), daunorubicin liposomal (daunorubicin citrate liposome, sold under the tradename DAUNOXOME), mitoxantrone (also known as DHAD, sold under the tradename NOVANTRONE), epirubicin (sold under the tradename ELLENCE), idarubicin (sold under the tradenames IDAMYCIN, IDAMYCIN PFS), and mitomycin C (sold under the tradename MUTAMYCIN).

Examples of anti-metabolites include, but are not limited to, claribine (2-chlorodeoxyadenosine, sold under the tradename LEUSTATIN), 5-fluorouracil (sold under the tradename ADRUCIL), 6-thioguanine (sold under the tradename PURINETHOL), pemetrexed (sold under the tradename ALIMTA), cytarabine (also known as arabinosylcytosine (Ara-C), sold under the tradename CYTOSAR-U), cytarabine liposomal (also known as Liposomal Ara-C, sold under the tradename DEPOCYT), decitabine (sold under the tradename DACOGEN), hydroxyurea (sold under the tradenames HYDREA, DROXIA and MYLOCEL), fludarabine (sold under the tradename FLUDARA), floxuridine (sold under the tradename FUDR), cladribine (also known as 2-chlorodeoxyadenosine (2-CdA) sold under the tradename LEUSTATIN), methotrexate (also known as amethopterin, methotrexate sodium (MTX), sold under the tradenames RHEUMATREX and TREXALL), and pentostatin (sold under the tradename NIPENT).

Examples of retinoids include, but are not limited to, alitretinoin (sold under the tradename PANRETIN), tretinoin (all-trans retinoic acid, also known as ATRA, sold under the tradename VESANOID), Isotretinoin (13-c/s-retinoic acid, sold under the tradenames ACCUTANE, AMNESTEEM, CLARAVIS, CLARUS, DECUTAN, ISOTANE, IZOTECH, ORATANE, ISOTRET, and SOTRET), and bexarotene (sold under the tradename TARGRETIN).

In such combinations the compound of the present invention and other active agents may be administered separately or in conjunction. In addition, the administration of one agent may be prior to, concurrent to, or subsequent to the administration of other agent(s).

### EXAMPLES

The meanings of the abbreviations in Examples are shown below.
ACN=CH₃CN=MeCN=acetonitrile
Ac=acetylv
AcOH=acetic acid
AIBN=azobisisobutyronitrile
APhos-Pd-G3=Palladium G3-(4-(N,N-Dimethylamino)phenyl)di-tert-butylphosphine=[4-(Di-tert-butylphosphino)-N,N-dimethylaniline-2-(2′-aminobiphenyl)]palladium(II) methanesulfonate
APhos-Pd-G4=4-Ditert-butylphosphanyl-N,N-dimethylaniline; methanesulfonic acid; N-methyl-2-phenylaniline; palladium
Boc=tert-butyloxycarbonyl
Boc-Ser(Bzl)-OH=N-(tert-Butoxycarbonyl)-O-benzyl-L-serine
BPO=benzoyl peroxide
BrettPhos Pd G3=[(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate
cataCXium A®=Di(1-adamantyl)-n-butylphosphine
CDI=1,1′-carbonyldiimidazole
CELITE=diatomaceous earth
CF₃CH₂OH=2,2,2-trifluoroethanol
Conc.=concentrated
CO₂=carbon dioxide
Cs₂CO₃=cesium carbonate
DCE=dichloroethane
DCM=dichloromethane
DIEA=DIPEA=N,N-diisopropylethylamine=Hünig's base
DMA=Dimethylacetamide
DMAP=4-Dimethylaminopyridine
DMF=N,N-dimethylformamide
DMSO=dimethyl sulfoxide
DPPE=1,2-bis(diphenylphosphino)ethane
EDCI=1-ethyl-3-(3-dimethylaminopropyl)carbodiimide
EtOAc=ethyl acetate
EtOH=ethanol
h=hours
H₂=hydrogen
H₂O=water
HATU=1-[bis(dimethylamino)methylene]-1H-1,2,3-triazolo[4,5-b]pyridinium 3-oxid hexafluorophosphate
HBr=hydrogen bromide
HCl=hydrochloric acid
HFBA=heptafluorobutyric acid
HOBT=hydroxybenzotriazole
HPLC=high pressure liquid chromatography
K₂CO₃=potassium carbonate
K₃PO₄=tripotassium phosphate
LCMS=liquid chromatography-mass spectrometry
LHMDS=LiHMDS=lithium bis(trimethylsilyl)amide
LiAlH₄=LAH=lithium aluminum hydride
LG=leaving group
LiF=lithium fluoride
LiOH=lithium hydroxide
min=minutes
MeOH=methanol
MgSO₄=magnesium sulfate
Ms=mesyl
MTBE=methyl tert-butyl ether
NaBH₄=sodium borohydride
NaCl=sodium chloride
NaHCO₃=sodium bicarbonate
NaOH=sodium hydroxide
Na₂SO₄=sodium sulfate
NaH=sodium hydride
NBS=N-Bromosuccinimide
NCS=N-Chlorosuccinimide
NH₄Cl=ammonium chloride
NH₄OH=ammonium hydroxide
NMP=N-methyl-2-pyrrolidone
OMs=mesylate
OTs=tosylate
OTf=triflates
Pd—C=palladium on carbon
Pd(OH)₂/C=Pearlman's catalysts=palladium hydroxide on carbon
Pd₂(dba)₃=Tris(dibenzylideneacetone)dipalladium(0)
Pd(dppf)Cl₂=[1,1′-Bis(diphenylphosphino)ferrocene]palladium(II) dichloride
Pd (dtbpf) Cl₂=[1,1′-Bis(di-tert-butylphosphino)ferrocene]dichloropalladium(II)
RuPhos Pd G3=(2-Dicyclohexylphosphino-2′,6′-diisopropoxy-1,1′-biphenyl)[2-(2′-amino-1,1′-biphenyl)]palladium(II) methanesulfonate=RuPhos-G3-Palladacycle
SFC=supercritical fluid chromatography
sSPhos Pd G2=chloro(sodium-2-dicyclohexylphosphino-2′,6′-dimethoxy-1,1′-biphenyl-3′-sulfonate)[2-(2′-amino-1,1′-biphenyl)]palladium(II)
TBAF=tetrabutyl ammonium fluoride
TEA=triethylamine
Tf=tryfly
TFA=trifluoroacetic acid
TFAA=trifluoroacetic anhydride
THF=tetrahydrofuran
Ts=tosyl
Xphos-Pd G2=Chloro(2-dicyclohexylphosphino-2′,4′,6′-triisopropyl-1,1′-biphenyl)[2-(2′-amino-1,1′-biphenyl)]palladium(II)=X-Phos aminobiphenyl palladium chloride precatalyst
1 Standard atmosphere [atm]=101325 pascal [Pa]=14.6959488 psi

The meanings of the abbreviations in the nuclear magnetic resonance spectra are shown below:
s=singlet, d=doublet, dd=double doublet, dt=double triplet, ddd=double double doublet, Sept=septet, t=triplet, m=multiplet, br=broad, brs=broad singlet, q=quartet
J=coupling constant and Hz=hertz.

Compounds of this invention can be prepared using the intermediates and processes outlined below. The various starting materials used are commercially available or are readily made by a person skilled in the art.

### Schemes

In general, intermediates and compounds of Formula I were synthesized according to one of Schemes 1-34.

### Scheme 1

Certain intermediates of compounds of Formula I were synthesized by converting benzylic alcohol 1 to the mesylate 2 by reaction with methanesulfonyl chloride in the presence of base.

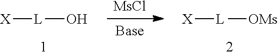

### Scheme 2

Certain intermediates of compounds of Formula I were synthesized by the reductive amination of amine 3 with various ketones to afford the protected diamine 4. Reaction of diamine 4 with methyl 2-chloro-2-oxoacetate followed by acidic deprotection affords the free amine 6 which was then cyclized in the presence of base to afford the diketopiperazine 7.

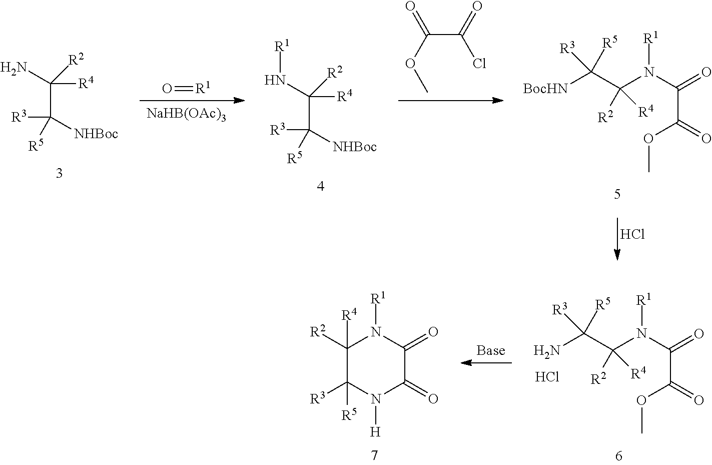

### Scheme 3

Certain intermediates of compounds of Formula I were synthesized by first reacting aryl bromide 8 with aryl boronic acid 9 under palladium catalyzed Suzuki conditions. The resulting biaryl 10 was converted to the bromide 11 by reaction with NBS and AIBN.

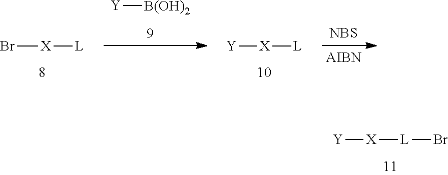

### Scheme 4

Certain intermediates of compounds of Formula I were synthesized by first reacting aryl bromide 12 with aryl boronic acid 9 under palladium catalyzed Suzuki conditions. The resulting biaryl benzylic alcohol 13 was converted to the benzylic bromide 14 by reaction with carbon tetrabromide and triphenylphosphine.

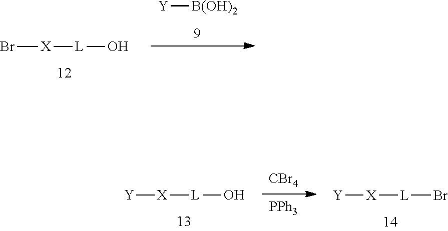

### Scheme 5

Certain intermediates of compounds of Formula I were synthesized by first reacting aryl bromide 8 with aryl boronic acid 9 under palladium catalyzed Suzuki conditions. The resulting biaryl 10 was converted to the bromide 11 by reaction with NBS and BPO.

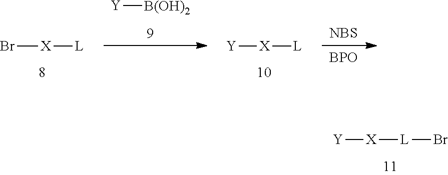

### Scheme 6

Certain intermediates of compounds of Formula I were synthesized by first reacting amine 15 with a 2-chloro-2-oxoacetate ester (where R=methyl or ethyl) to afford 16. Subsequent alkylation of 16 with alkyl bromide 17, followed by a separate step of oxidative cleavage afforded intermediate 19.

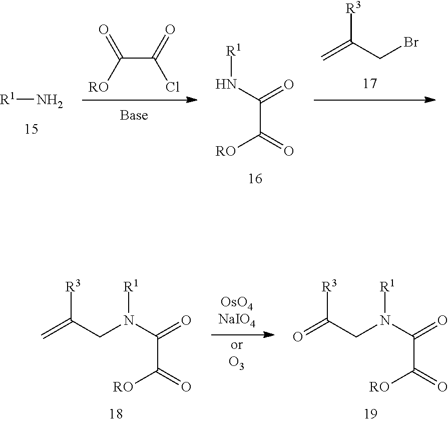

### Scheme 7

Certain intermediates of compounds of Formula I were synthesized by first reacting an aryl hydrazide 20 with either an α- or β-amino acid 21 to afford 22. Subsequent cyclization with Lawesson's reagent followed by a separate acid mediated deprotection afforded the substituted thiadiazole 23.

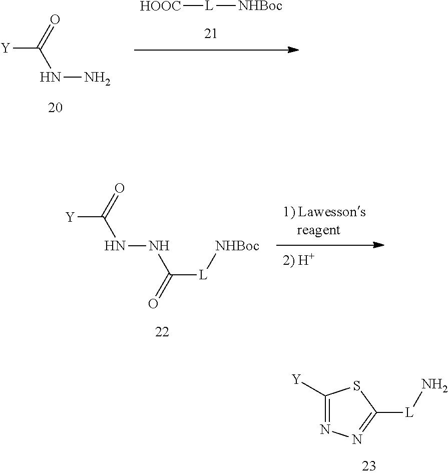

### Scheme 8

Certain intermediates of compounds of Formula I were synthesized by the reaction of amine 15 with methyl 2-chloro-2-oxoacetate to afford 24. Reaction of 24 with 2,2-dimethoxyethan-1-amine followed by a separate step of acidic deprotection/cyclization afforded the dihydro-diketopiperazine 26.

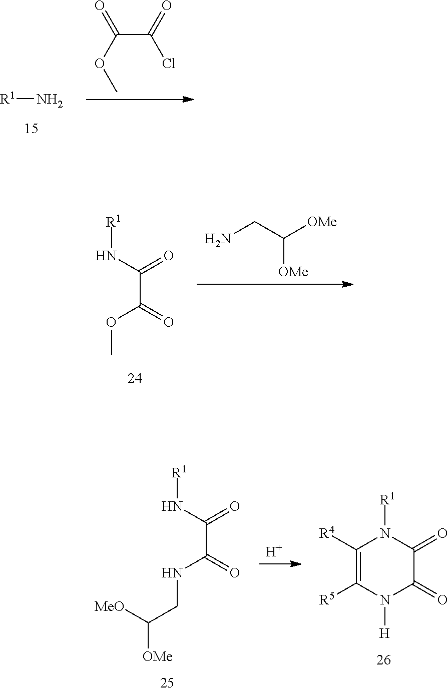

### Scheme 9

Certain intermediates of compounds of Formula I were synthesized by first reacting an aryl benzaldehyde oxime 27 with NCS to afford the hydroxybenzimidoyl chloride 28. Subsequent reaction with propargyl alcohol gives the isoxazole 29. The synthesis is completed by reaction with methanesulfonyl chloride to give the substituted isoxazole 30.

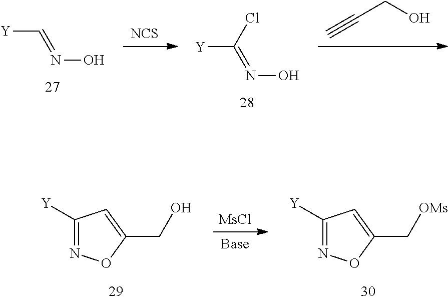

### Scheme 10

Certain intermediates of compounds of Formula I were synthesized by first reacting an ethynyl arene 31 with ethyl 2-chloro-2-(hydroxyimino)acetate to afford the substituted isoxazole 32. Reduction followed by mesylation afforded the substituted isoxazole 34.

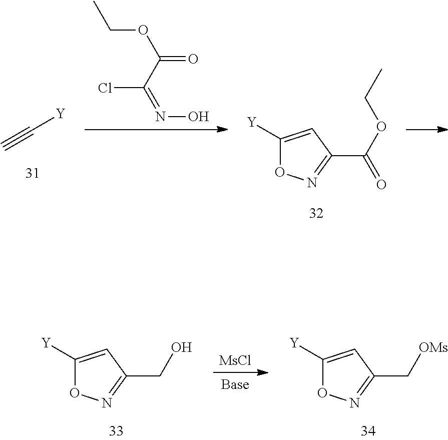

### Scheme 11

Certain intermediates of compounds of Formula I were synthesized by first reacting 3-chloro-6-methylpyridazine 35 with an aryl boronic acid 9 under palladium-catalyzed Suzuki conditions. The resulting biaryl 36 was converted to the benzyl chloride 37 by reaction with 1,3,5-trichloro-1,3,5-triazinane-2,4,6-trione.

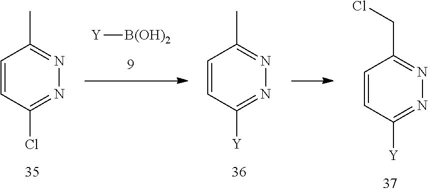

### Scheme 12

Certain intermediates of compounds of Formula I were synthesized by first reacting an aryl hydrazide 20 with chloroacetyl chloride to afford the hydrazide 38. Subsequent cyclization with Lawesson's reagent afforded the substituted thiadiazole 39.

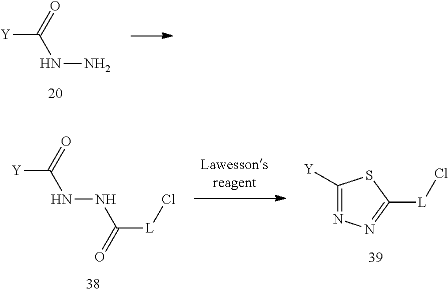

### Scheme 13

Certain intermediates of compounds of Formula I were synthesized by the alkylation of diketopiperazine 7 with various electrophiles 40 (where LG includes, but is not limited to, —Cl, —Br, —OMs, and —OTs) using either sodium hydride or a metal carbonate base to afford 41.

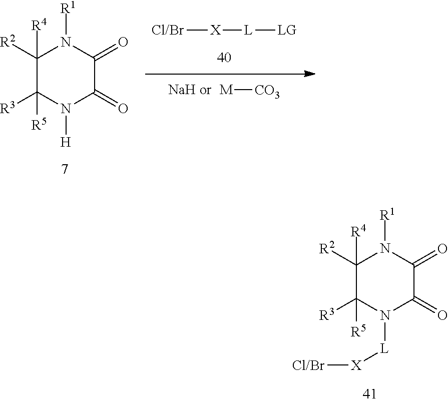

### Scheme 14

Certain intermediates of compounds of Formula I were synthesized by first reacting a bromoaryl nitrile 42 with aryl boronic acid 9 under palladium catalyzed Suzuki conditions. The resulting biaryl nitrile 43 was reduced by hydrogenation to the amine 44. Subsequent reaction with ethyl 2-chloro-2-oxoacetate afforded 45. Allylation followed by a separate step of ozonolysis afforded the aldehyde 47.

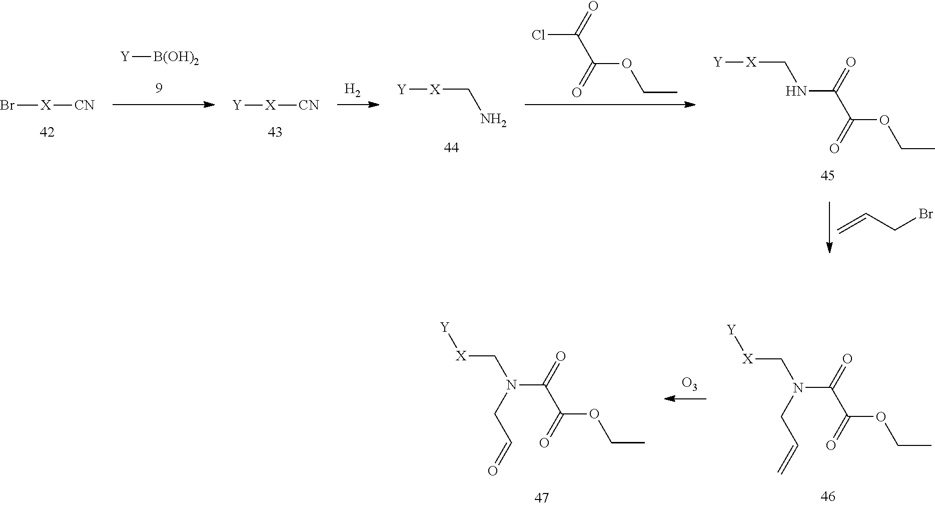

### Scheme 15

Certain intermediates of compounds of Formula I were synthesized by the reductive amination of aldehyde 48 with an amine 15 to give the substituted diketopiperazine 49.

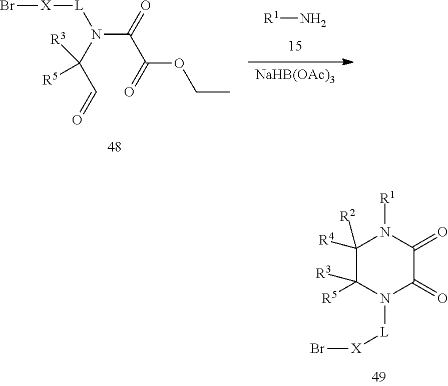

### Scheme 16

Certain intermediates of compounds of Formula I were synthesized by the amination of aldehyde 48 with an amine 15 to give the substituted dihydro-diketopiperazine 50.

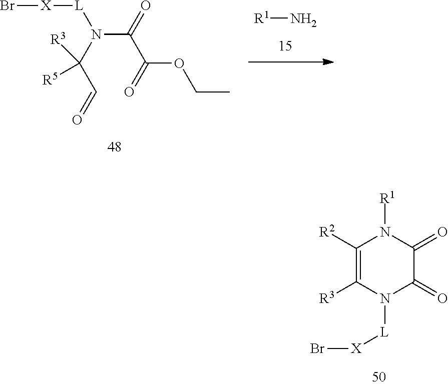

### Scheme 17

Certain intermediates of compounds of Formula I were synthesized by first reacting a biaryl benzylic chloride 51 with tert-butyl (2-aminoethyl)carbamate. The resulting diamine product 52 was reacted with ethyl 2-chloro-2-oxoacetate to afford 53. Subsequent BOC deprotection followed in a separate step by base-mediated cyclization afforded the diketopiperazine 55.

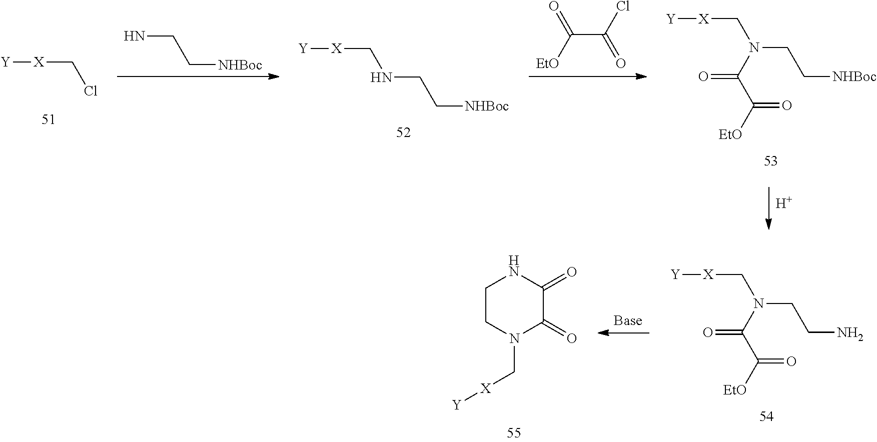

### Scheme 18

Certain intermediates of compounds of Formula I were synthesized by first reacting an aryl bromide 12 with a nitrogen-containing arene (Y) under copper-mediated conditions. The resulting biaryl 13 was converted to the chloride 56 by reaction with thionyl chloride.

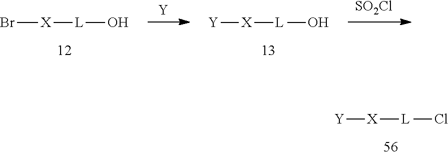

### Scheme 19

Certain intermediates of compounds of Formula I were synthesized by first reacting an aryl chloride 57 with a nitrogen-containing arene (Y) under SNAr conditions. The resulting biaryl 58 was converted to the chloride 56 by reaction with thionyl chloride.

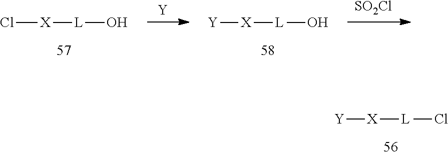

### Scheme 20

Certain intermediates of compounds of Formula I were synthesized by first reacting an aryl aldehyde 59 with hydroxylamine hydrochloride to afford 27. The aryl benzaldehyde oxime 27 was reacted with NCS to afford the hydroxybenzimidoyl chloride 28. Subsequent reaction with propargyl alcohol gives the isoxazole 29. The synthesis was completed by reaction with methanesulfonyl chloride to give the substituted isoxazole 30.

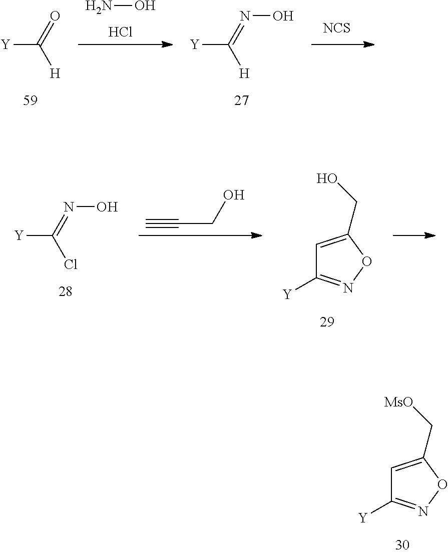

### Scheme 21

Certain compounds of Formula I were synthesized by reacting either the aryl chloride or aryl bromide 41 with aryl boronic acid 9 under palladium catalyzed Suzuki conditions.

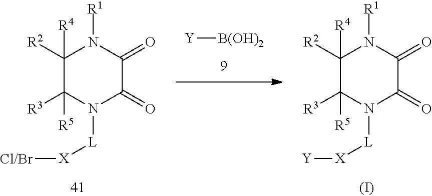

### Scheme 22

Certain compounds of Formula I were synthesized by alkylating the diketopiperazine 7 with an electrophile 60 (where LG includes, but is not limited to, —Cl, —Br, —OMs, and —OTf).

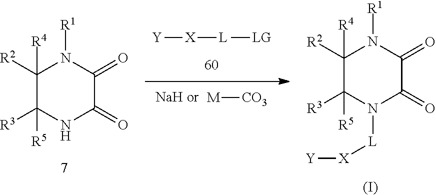

### Scheme 23

Certain compounds of Formula I were synthesized by coupling of a carboxylate 61 with an electrophile 62 (where LG includes, but is not limited to, halides and triflate) under photoredox conditions using catalytic iridium and nickel.

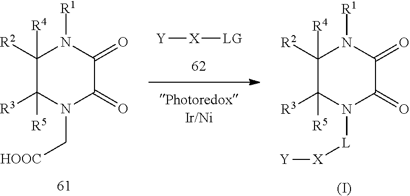

### Scheme 24

Certain compounds of Formula I were synthesized by the reductive amination of aldehyde 47 with an amine 15.

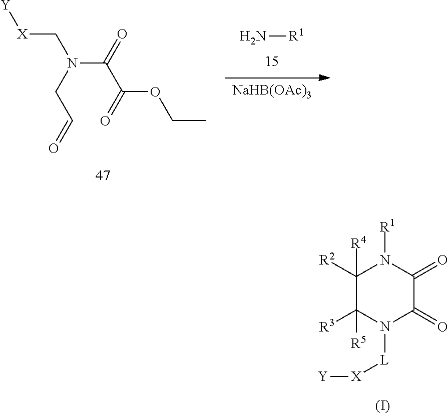

### Scheme 25

Certain compounds of Formula I were synthesized by an SNAr reaction where Y is a nucleophile and reacts with either the aryl chloride or aryl bromide 41 in the presence of either a

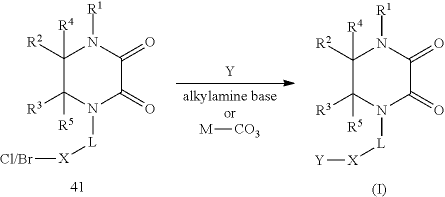

### Scheme 26

Certain compounds of Formula I were synthesized by the amination of aldehyde 47 with an amine 15.

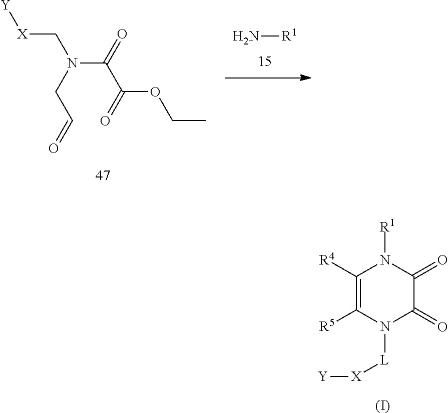

### Scheme 27

Certain compounds of Formula I were synthesized by reacting an alkyl boronate 63 with an aryl bromide 64 under palladium catalyzed Suzuki conditions.

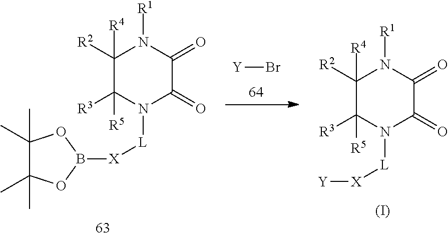

### Scheme 28

Certain compounds of Formula I were synthesized by the reductive amination of electrophile 19 with an amine 65.

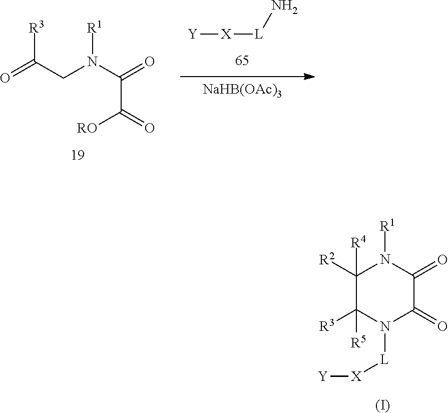

### Scheme 29

Certain compounds of Formula I were synthesized by reacting either the aryl chloride or aryl bromide 41 with an aryl stannane (where R=alkyl) 66 under palladium catalyzed Stille conditions.

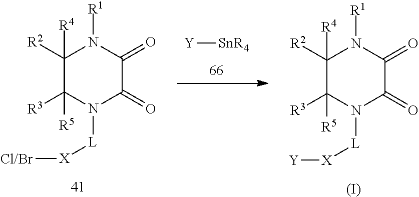

### Scheme 30

Certain compounds of Formula I were synthesized by reacting either the aryl chloride or aryl bromide 41 under copper mediated C—N coupling conditions, where Y contains an —NH.

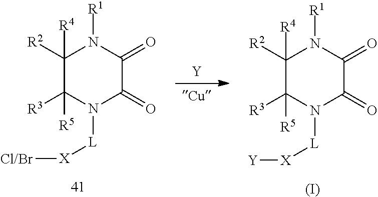

### Scheme 31

Certain compounds of Formula I were synthesized by reacting either the aryl chloride or aryl bromide 41 under palladium mediated C—N coupling conditions, where Y contains an —NH.

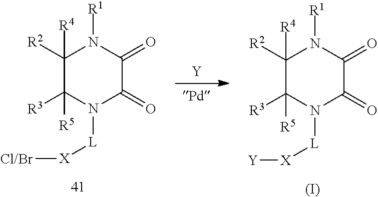

### Scheme 32

Certain compounds of Formula I were synthesized by reacting either the aryl chloride or aryl bromide 41 under palladium mediated C—H activation conditions, where Y is an heteroarene.

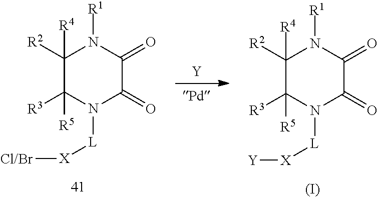

### Scheme 33

Certain compounds of Formula I were synthesized by reacting an aryl iodide 68 with the arene 67 under palladium mediated C—H activation conditions.

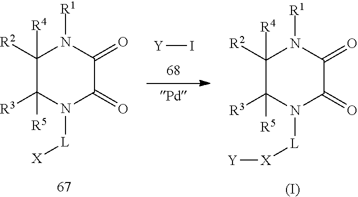

### Scheme 34

Certain compounds of Formula I were synthesized by reacting an aryl azide 69 with the arene 67 (wherein X=1,2,3-triazole) under copper-mediated coupling conditions.

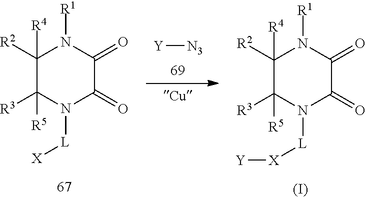

### Intermediate 1

### Preparation of 5-bromo-2-(bromomethyl)pyrimidine

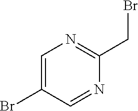

N-Bromosuccinimide (309 mg, 1.73 mmol) was added to a mixture of 5-bromo-2-methylpyrimidine (250 mg, 1.45 mmol) and azobisisobutyronitrile (AIBN) (71 mg, 0.43 mmol) in carbon tetrachloride (5 mL) at room temperature. The reaction mixture was stirred and heated to 80° C. for 12 hours. The reaction mixture was cooled to room temperature and concentrated under reduced pressure. The residue was purified by silica gel chromatography (eluting ethyl acetate in petroleum ether) to afford 5-bromo-2-(bromomethyl)pyrimidine. LC/MS (m/z): 253 (M+H)⁺

### Intermediate 2

### Preparation of bicyclo[3.1.0]hexan-3-amine

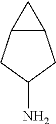

### Step A: Benzyl cyclopent-3-en-1-ylcarbamate

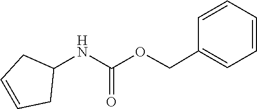 [SMILES: O=C(NC1CC=CC1)OCc1ccccc1]

Benzyl chloroformate (1.43 mL, 10.0 mmol) was added to a mixture of cyclopent-3-enamine hydrochloride (1.00 g, 8.36 mmol), triethylamine (3.50 mL, 25.1 mmol), and DMAP (0.102 g, 0.836 mmol) in DCM (20 mL) at 0° C. under a nitrogen atmosphere. The reaction mixture was warmed to 20° C. and stirred for 14 hours. The reaction mixture was washed with brine, dried over anhydrous sodium sulfate, filtered, and concentrated under reduced pressure. The residue was purified by silica gel chromatography (eluting ethyl acetate in petroleum ether) to afford benzyl cyclopent-3-en-1-ylcarbamate. ¹H NMR (400 MHz, chloroform-d) δ 7.39-7.26 (m, 5H), 5.67 (s, 2H), 5.07 (s, 2H), 4.93 (br s, 1H), 4.39-4.24 (m, 1H), 2.75-2.69 (m, 2H), 2.23-2.13 (m, 2H). LC/MS (m/z): 218 (M+H)⁺

### Step B: Benzyl bicyclo[3.1.0]hexan-3-ylcarbamate

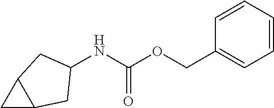 [SMILES: O=C(NC1CC2CC2C1)OCc1ccccc1]

A solution of diethylzinc in diethyl ether (1.0 M, 2.9 mL, 2.9 mmol) was added dropwise to a mixture of benzyl cyclopent-3-en-1-ylcarbamate (250 mg, 1.15 mmol) in DCM (10 mL) at 0° C. under a nitrogen atmosphere. Diiodomethane (770 mg, 2.88 mmol) was then added dropwise to the reaction mixture at 0° C. under a nitrogen atmosphere. The reaction mixture was warmed to room temperature and stirred for an additional four hours. The resulting mixture was quenched with saturated aqueous ammonium chloride (10 mL) and extracted with ethyl acetate (2×20 mL). The organic layers were combined, washed with brine (20 mL), dried over anhydrous sodium sulfate, filtered, and concentrated under reduced pressure. The residue was purified by reverse phase HPLC (eluting acetonitrile in water, with TFA modifier) to afford benzyl bicyclo[3.1.0]hexan-3-ylcarbamate. ¹H NMR (400 MHz, chloroform-d) δ 7.33-7.25 (m, 5H), 5.01 (s, 2H), 4.44 (br s, 1H), 4.17-4.07 (m, 1H), 2.32-2.15 (m, 2H), 1.55 (dd, J=14.1 Hz, J=1.8 Hz, 2H), 1.27-1.21 (m, 2H), 0.60-0.51 (m, 1H), 0.00 (br s, 1H). LC/MS (m/z): 232 (M+H)⁺

### Step C: Bicyclo[3.1.0]hexan-3-amine

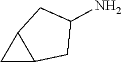

A mixture of benzyl bicyclo[3.1.0]hexan-3-ylcarbamate (1.1 g, 4.8 mmol) and 5% Pd/C (0.506 g) was sparged with nitrogen. MeOH (15 mL) was added, and the mixture was degassed and backfilled with hydrogen gas (3×). The reaction mixture was stirred under a hydrogen atmosphere (15 psi) at room temperature for two hours. The reaction mixture was filtered, and the filtrate was diluted with HCl (4 M in 1,4-dioxane, 1 mL, 4 mmol) and stirred for 20 minutes. The reaction mixture was concentrated under reduced pressure to afford bicyclo[3.1.0]hexan-3-amine as a hydrochloride salt, which was used without purification in the next step. ¹H NMR (400 MHz, methanol-d₄) δ 3.68-3.55 (m, 1H), 2.30-2.23 (m, 2H), 1.41-1.33 (m, 2H), 1.24-1.20 (m, 2H), 0.72-0.60 (m, 1H), 0.10-0.01 (m, 1H).

### Intermediate 3

### Preparation of (5-bromopyridin-2-yl)methyl methanesulfonate

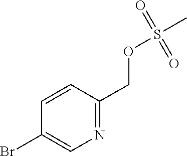

Triethylamine (1.69 mL, 12.1 mmol) was added dropwise to a mixture of (5-bromopyridin-2-yl)methanol (1.90 g, 10.1 mmol) and methanesulfonic anhydride (1.94 g, 11.1 mmol) in dichloromethane (40 mL) at 0° C. The reaction mixture was stirred at 0° C. for 15 minutes after addition was complete. The reaction mixture was quenched with water (20 mL). The organic layer was separated, washed with brine (25 mL), dried over magnesium sulfate, filtered, and concentrated under reduced pressure. The residue was purified by silica gel chromatography (eluting ethyl acetate in hexanes) to afford (5-bromopyridin-2-yl)methyl methanesulfonate. LC/MS (m/z): 266, 268 (M+H)⁺

### Intermediate 4

### Preparation of (5-bromo-3-fluoropyridin-2-yl)methyl methanesulfonate

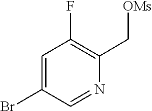 [SMILES: CS(=O)(=O)OCc1ncc(Br)cc1F]

Methanesulfonyl chloride (0.17 mL, 2.2 mmol) was added to a mixture of (5-bromo-3-fluoropyridin-2-yl)methanol (371 mg, 1.80 mmol) and Hunig's base (0.472 mL, 2.70 mmol) in DCM (6 mL) at room temperature. The reaction mixture was stirred at room temperature for 1 hour. The reaction mixture was diluted with DCM (30 mL) and water (30 mL). The organic layer was dried over anhydrous sodium sulfate, filtered, and concentrated under reduced pressure to afford (5-bromo-3-fluoropyridin-2-yl)methyl methanesulfonate, which was used without purification in the next step. LC/MS (m/z): 284, 286 (M+H)⁺.

Intermediates shown in Intermediate Table 1 below were prepared according to procedures analogous to those outlined in Intermediate 4 above using the appropriate starting materials, described in the Preparations or Intermediates above, or as obtained from commercial sources.

Intermediate Table 1

| | | | Mass |
|---|---|---|---|
| Intermediate | Structure | Name | [M + H]⁺ |
| Intermediate 5 | 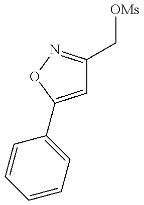 [SMILES: CS(=O)(=O)OCc1cc(-c2ccccc2)on1] | (5-phenylisoxazol- 3-yl) methyl methanesulfonate | 254 |

### Intermediate 6

### Preparation of tert-butyl (2-(((cis)-bicyclo[3.1.0]hexan-3-yl)amino)ethyl)carbamate

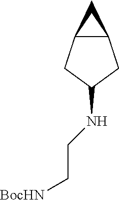

A mixture of tert-butyl (2-bromoethyl)carbamate (384 g, 1.81 mol), bicyclo[3.1.0]hexan-3-amine hydroiodide (500 g, 1.71 mol), and potassium carbonate (474 g, 4.23 mol) in DMF (2.50 L) was stirred and heated at 60° C. for 12 hours. The reaction mixture was cooled to room temperature. The reaction mixture was quenched by the addition of water (5.0 L) at room temperature and then diluted with EtOAc (700 mL). The organic layer was separated. The aqueous layer was extracted with additional EtOAc (3×700 mL). The organic layers were combined, washed with brine (3×1.0 L), dried over anhydrous Na₂SO₄, filtered, and concentrated under reduced pressure to afford tert-butyl (2-(((cis)-bicyclo[3.1.0]hexan-3-yl)amino)ethyl)carbamate, which was used without purification. LC/MS (m/z): 241 (M+H)⁺

### Intermediate 7

### Preparation of (5-bromoisoxazol-3-yl)methyl methanesulfonate

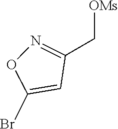 [SMILES: CS(=O)(=O)OCc1cc(Br)on1]

### Step A: ethyl 5-(tributylstannyl)isoxazole-3-carboxylate

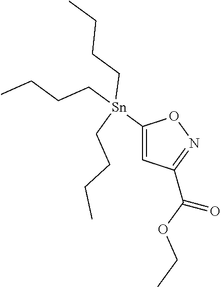 [SMILES: CCC[CH2][Sn]([CH2]CCC)([CH2]CCC)[c]1cc(C(=O)OCC)no1]

Potassium carbonate (2.41 g, 17.4 mmol) and tributyl(ethynyl)stannane (4.99 g, 15.8 mmol) were added to a mixture of ethyl (Z)-2-chloro-2-(hydroxyimino)acetate (2.40 g, 15.8 mmol) in DCM (40 mL) at room temperature. The reaction mixture was stirred at room temperature for two days. The reaction mixture was diluted with water (50 mL) and dichloromethane (250 mL). The organic layer was separated, washed with additional water (50 mL) and brine (50 mL). The organic layer was separated, dried over magnesium sulfate, filtered, and concentrated under reduced pressure. The residue was purified by silica gel chromatography (eluting ethyl acetate in hexanes) to afford ethyl 5-(tributylstannyl)isoxazole-3-carboxylate. LC/MS (m/z): 432 (M+H)⁺

### Step B: ethyl 5-bromoisoxazole-3-carboxylate

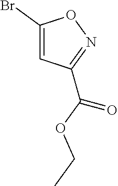

Bromine (0.952 mL, 18.5 mmol) was added to a mixture of ethyl 5-(tributylstannyl)isoxazole-3-carboxylate (5.30 g, 12.3 mmol) and sodium carbonate (1.50 g, 14.2 mmol) in DCM (75 mL) at room temperature. The reaction mixture was stirred at room temperature for 1 day. The reaction mixture was quenched by the addition of saturated aqueous sodium thiosulfate (20 mL) and then diluted with water (50 mL) and dichloromethane (200 mL). The organic layer was separated and washed with brine (50 mL). The organic layer was separated, dried over magnesium sulfate, filtered, and concentrated under reduced pressure. The residue was purified by silica gel chromatography (eluting ethyl acetate in hexanes) to afford ethyl 5-bromoisoxazole-3-carboxylate. ¹H NMR (499 MHz, DMSO-d₆) δ 7.24 (s, 1H), 4.38 (q, J=7.1 Hz, 2H), 1.32 (t, J=7.1 Hz, 3H).

### Step C: (5-bromoisoxazol-3-yl)methanol

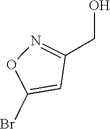

Diisobutylaluminium hydride (DIBAL-H) (1.0 M in hexanes, 20 mL, 20 mmol) was added slowly to a mixture of ethyl 5-bromoisoxazole-3-carboxylate (1.89 g, 8.59 mmol) in THE (20 mL) at 0° C. The reaction mixture was stirred at 0° C. for 1 hour. The reaction mixture was quenched by the slow addition of HCl (6.0 M in water, 6.6 mL, 40 mmol) at 0° C. The mixture was stirred for 2 hours and then diluted with water (10 mL) and dichloromethane (100 mL), and then warmed to room temperature. The organic layer was separated, washed with brine (10 mL), dried over magnesium sulfate, filtered, and concentrated under reduced pressure to afford (5-bromoisoxazol-3-yl)methanol which was used without purification in the next step. LC/MS (m/z): 178, 180 (M+H)⁺

### Step D: (5-bromoisoxazol-3-yl)methyl methanesulfonate

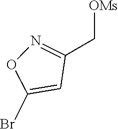 [SMILES: CS(=O)(=O)OCc1cc(Br)on1]

Methanesulfonyl chloride (0.79 mL, 10 mmol) was added to a mixture of (5-bromoisoxazol-3-yl)methanol (1.50 g, 8.43 mmol) and triethylamine (1.65 mL, 11.8 mmol) in DCM (30 mL) at 0° C. The reaction was stirred at 0° C. for 10 minutes. The mixture was concentrated under reduced pressure, and the residue was purified by silica gel chromatography (eluting [3:1 EtOAc:EtOH] in hexanes) to afford (5-bromoisoxazol-3-yl)methyl methanesulfonate. LC/MS (m/z): 256, 258 (M+H)⁺

### Intermediate 8

### Preparation of isoxazol-3-ylmethyl methanesulfonate

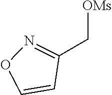 [SMILES: CS(=O)(=O)OCc1ccon1]

### Step A: isoxazol-3-ylmethanol

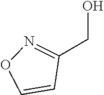

Borane-THF (1.0 M in THF, 26.5 mL, 26.5 mmol) was added to a mixture of isoxazole-3-carboxylic acid (1.00 g, 8.84 mmol) in THE (10 ml) at 0° C. The mixture was warmed to room temperature and stirred for 12 hours. The reaction was quenched with MeOH (8 mL) and stirred at room temperature for 2 hours. The mixture was diluted with water and extracted with EtOAc (3×50 mL), dried over anhydrous Na₂SO₄, filtered, and concentrated under reduced pressure to afford isoxazol-3-ylmethanol, which was used in the next step without purification.

### Step B: isoxazol-3-ylmethyl methanesulfonate

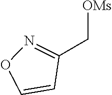 [SMILES: CS(=O)(=O)OCc1ccon1]

Methanesulfonic anhydride (1.32 g, 7.57 mmol) was added to a mixture of isoxazol-3-ylmethanol (500 mg, 5.05 mmol) and TEA (1.41 ml, 10.1 mmol) in DCM (20 mL) at room temperature. The mixture was stirred at room temperature for 1 hour. The mixture was concentrated under reduced pressure to afford isoxazol-3-ylmethyl, which was used in the next step without purification. LC/MS (m/z): 178 (M+H)⁺

### Intermediate 9

### Preparation of cyclopentan-1-d-1-amine

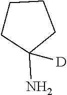

### Step A: N-cyclopentylidene-2-methylpropane-2-sulfinamide

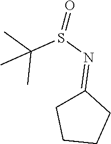

2-methylpropane-2-sulfinamide (1.59 g, 13.1 mmol) was added to a mixture of cyclopentanone (1.00 g, 11.9 mmol) and titanium (IV) ethoxide (4.89 ml, 23.78 mmol) in THE (20 ml) at room temperature. The mixture was stirred for 12 hours at room temperature. The mixture was concentrated under reduced pressure, and the residue was partitioned between water (10 mL) and EtOAc (10 mL). The organic layer was separated, and the aqueous was re-extracted with EtOAc (3×10 mL). The organic layers were combined, washed with brine (10 mL), dried over anhydrous Na₂SO₄, filtered, and concentrated under reduced pressure to afford N-cyclopentylidene-2-methylpropane-2-sulfinamide, which was used in the next step without purification. LC/MS (m/z): 188 (M+H)⁺

### Step B: N-(cyclopentyl-1-d)-2-methylpropane-2-sulfinamide

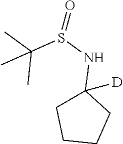

A mixture of N-cyclopentylidene-2-methylpropane-2-sulfinamide (1.00 g, 5.34 mmol) and sodium borodeuteride (0.402 g, 9.61 mmol) in MeOD (15 mL) was degassed and backfilled with N₂ (three times) and then stirred at room temperature for 12 hours. The mixture was quenched with water and then partitioned between water (30 mL) and EtOAc (300 mL). The organic layer was separated, and the aqueous was re-extracted with EtOAc (3×100 mL). The organic layers were combined, washed with brine (30 mL), dried over anhydrous Na₂SO₄, filtered, and concentrated under reduced pressure. The residue was purified by reverse phase HPLC (eluting acetonitrile in water with TFA modifier) to afford N-(cyclopentyl-1-d)-2-methylpropane-2-sulfinamide. LC/MS (m/z): 191 (M+H)⁺

### Step C: cyclopentan-1-d-1-amine

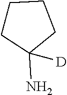

A mixture of N-(cyclopentyl-1-d)-2-methylpropane-2-sulfinamide (150 mg, 0.788 mmol) and hydrochloric acid (4.0 M in dioxane, 0.60 mL, 2.4 mmol) in 1,4-dioxane (5 mL) was stirred at room temperature for 2 hours. The mixture was concentrated under reduced pressure to afford cyclopentan-1-d-1-amine, which was used in the next step without purification.

### Intermediate 10

### Preparation of 1-cyclobutylpiperazine-2,3-dione

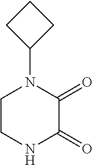 [SMILES: O=C1NCCN(C2CCC2)C1=O]

### Step A: tert-butyl (2-cyclobutylamino)ethyl)carbamate

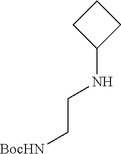 [SMILES: CC(C)(C)OC(=O)NCCNC1CCC1]

A mixture of cyclobutanone (273 g, 3.90 mol) and tert-butyl (2-aminoethyl)carbamate (625 g, 3.90 mol) in 1,2-dichloroethane (2.50 L) was cooled to 0° C. Sodium triacetoxyborohydride (1.16 kg, 5.46 mol) was added portion-wise to the mixture at 0° C. over a period of 1 hour. The mixture was warmed to room temperature and stirred for an additional 11 hours. The mixture was added to water (1.5 L) and the pH was adjusted to ˜8 by the addition of saturated aqueous sodium bicarbonate (1.5 L). The mixture was extracted with dichloromethane (2×1.5 L). The organic layers were combined, washed with brine (1 L), dried over anhydrous sodium sulfate, filtered, and concentrated under reduced pressure to afford tert-butyl (2-(cyclobutylamino)ethyl)carbamate, which was used in the subsequent step without purification.

### Step B: methyl 2-((2-((tert-butoxycarbonyl)amino)ethyl)(cyclobutyl)amino)-2-oxoacetate

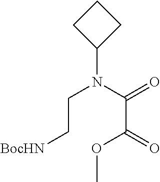 [SMILES: COC(=O)C(=O)N(CCNC(=O)OC(C)(C)C)C1CCC1]

A mixture of tert-butyl (2-(cyclobutylamino)ethyl)carbamate (600 g, 1.12 mol) and sodium bicarbonate (470 g, 5.60 mol) in dichloromethane (2.4 L) was cooled to 0° C. Methyl 2-chloro-2-oxoacetate (178 g, 1.46 mol) was added dropwise over a period of 1 hour at 0° C. The mixture was warmed to room temperature and stirred for an additional 11 hours. The mixture was then added to water (1.5 L). The organic layer was separated and washed with HCl (1.0 N, 1 L) and brine (1 L), dried over anhydrous Na₂SO₄, filtered, and concentrated under reduced pressure. The residue was purified by silica gel chromatography (eluting ethyl acetate in petroleum ether) to afford methyl 2-((2-((tert-butoxycarbonyl)amino)ethyl)(cyclobutyl)amino)-2-oxoacetate.

### Step C: methyl 2-((2-aminoethyl)(cyclobutyl)amino)-2-oxoacetate hydrochloride

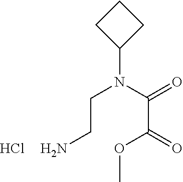

HCl (4.0 M in methanol, 3.6 mol, 900 mL) was added to a mixture of methyl 2-((2-((tert-butoxycarbonyl)amino)ethyl)(cyclobutyl)amino)-2-oxoacetate (300 g, 998 mmol) in methanol (600 mL) at room temperature. The mixture was stirred at room temperature for 12 hours and then concentrated under reduced pressure to afford methyl 2-((2-aminoethyl)(cyclobutyl)amino)-2-oxoacetate hydrochloride, which was used without purification in the next step. LC/MS (m/z): 201 (M+H)⁺

### Step D: 1-cyclobutylpiperazine-2,3-dione

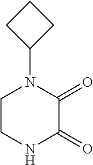 [SMILES: O=C1NCCN(C2CCC2)C1=O]

A mixture of methyl 2-((2-aminoethyl)(cyclobutyl)amino)-2-oxoacetate hydrochloride (350 g, 1.48 mol) and NaHCO₃ (434 g, 5.18 mol) in methanol (2.1 L) was stirred at room temperature for three hours. The mixture was concentrated under reduced pressure, and the residue was purified by silica gel chromatography (eluting methanol in dichloromethane) to afford 1-cyclobutylpiperazine-2,3-dione. ¹H NMR (400 MHz, DMSO-d₆) δ 8.54 (s, 1H), 4.80-4.71 (m, 1H), 3.52-3.49 (m, 2H), 3.33-3.23 (m, 2H), 2.17-2.02 (m, 4H), 1.66-1.63 (m, 2H). LC/MS (m/z): 169 (M+H)⁺

### Intermediate 11

### Preparation of 1-cyclopentylpiperazine-2,3-dione

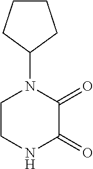 [SMILES: O=C1NCCN(C2CCCC2)C1=O]

### Step A: tert-Butyl (2-(cyclopentylamino)ethyl)carbamate

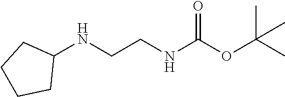 [SMILES: CC(C)(C)OC(=O)NCCNC1CCCC1]

A mixture of cyclopentanone (250 g, 2.97 mol) and tert-butyl (2-aminoethyl)carbamate (524 g, 3.27 mol) in methanol (1.75 L) was stirred at room temperature for 6 minutes. Sodium triacetoxyborohydride (882 g, 4.16 mol) was added portion wise to the reaction mixture at room temperature over a period of 2 hours. The reaction mixture was then stirred at room temperature for 3 hours after the addition was complete. The reaction mixture was diluted with methyl tert-butyl ether (MTBE) (5.00 L) and the resulting mixture was then filtered (washing the collected solids with additional methyl tert-butyl ether (500 mL)). The combined filtrates were concentrated under reduced pressure to afford tert-butyl (2-(cyclopentylamino)ethyl)carbamate, which was used in the next step without purification. ¹H NMR (400 MHz, DMSO-d₆) δ 7.04-6.89 (m, 1H), 3.08-3.04 (m, 4H), 2.71-2.63 (m, 2H), 1.72-1.63 (m, 2H), 1.63-1.61 (m, 2H), 1.62-1.48 (m, 2H), 1.45-1.28 (m, 11H). LC/MS (m/z): 229 (M+H)⁺

### Step B: Methyl 2-((2-((tert-butoxycarbonyl)amino)ethyl)(cyclopentyl)amino)-2-oxoacetate

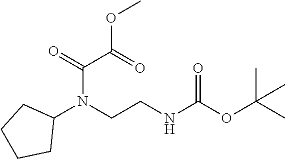 [SMILES: COC(=O)C(=O)N(CCNC(=O)OC(C)(C)C)C1CCCC1]

A mixture of tert-butyl (2-(cyclopentylamino)ethyl)carbamate (400 g, 1.75 mol) and triethylamine (532 g, 5.26 mol) in dichloromethane (3.2 L) was stirred for 6 minutes at room temperature. Methyl 2-chloro-2-oxoacetate (492 g, 4.03 mol) was added dropwise to the reaction mixture at room temperature over a period of two hours. The reaction mixture was stirred at room temperature for an additional two hours after the addition was complete. The reaction mixture was diluted with water (700 mL) and the organic layer was separated, dried over anhydrous sodium sulfate, filtered, and concentrated under reduced pressure. The residue was purified by silica gel chromatography (eluting ethyl acetate in petroleum ether) to afford methyl 2-((2-((tert-butoxycarbonyl)amino)ethyl)(cyclopentyl)amino)-2-oxoacetate. ¹H NMR (400 MHz, DMSO-d₆) δ 6.99-6.92 (m, 1H), 3.80-3.78 (m, 4H), 3.19-3.16 (m, 2H), 3.07-3.05 (m, 2H), 1.90-1.50 (m, 8H), 1.40-1.37 (m, 9H). LC/MS (m/z): 215 (M+H-Boc)⁺

### Step C: Methyl 2-((2-aminoethyl)(cyclopentyl)amino)-2-oxoacetate

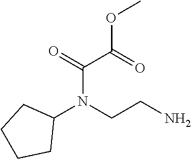

HCl (4.0 M in methanol, 1.2 L, 4.8 mol) was added to a mixture of methyl 2-((2-((tert-butoxycarbonyl)amino)ethyl)(cyclopentyl)amino)-2-oxoacetate (300 g, 0.95 mol) in methanol (0.6 L) at room temperature. The reaction mixture was stirred at room temperature for 6 hours. The reaction mixture was concentrated under reduced pressure to afford methyl 2-((2-aminoethyl)(cyclopentyl)amino)-2-oxoacetate as an HCl salt and was used without purification in the next step. ¹H NMR (400 MHz, DMSO-d₆) δ 8.35-8.26 (m, 3H), 3.86-3.82 (m, 4H), 3.53-3.45 (m, 2H), 2.88 (br s, 2H), 1.79-1.50 (m, 8H).

### Step D: 1-Cyclopentylpiperazine-2,3-dione

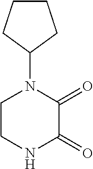 [SMILES: O=C1NCCN(C2CCCC2)C1=O]

Triethylamine (124 g. 1.22 mol) was added dropwise to a mixture of methyl 2-((2-aminoethyl)(cyclopentyl)amino)-2-oxoacetate hydrochloride (150 g, 598 mmol) in methanol (3.0 L) at room temperature. The reaction mixture was stirred at room temperature for 30 minutes. The reaction mixture was concentrated under reduced pressure. The residue was diluted with HCl (1.0 M in water, 2.0 L, 2.0 mol) and then extracted with a mixture of isopropyl alcohol/dichloromethane (1:10 mixture, 5×1.0 L). The organic layers were combined, washed with brine (1.0 L), dried over anhydrous sodium sulfate, filtered, and concentrated to afford 1-cyclopentylpiperazine-2,3-dione, which was used without purification. ¹H NMR (400 MHz, methanol-d₄) δ 4.84-4.78 (m, 1H), 3.54-3.52 (m, 2H), 3.47-3.31 (m, 2H), 1.90-1.88 (m, 2H), 1.78-1.77 (m, 2H), 1.64-1.61 (m, 4H). LC/MS (m/z): 183 (M+H)⁺

Intermediates shown in Intermediate Table 2 below, were prepared according to procedures analogous to those outlined in Intermediate 11 above using the appropriate starting materials, described in the Preparations or Intermediates above, or as obtained from commercial sources.

Intermediate Table 2

| Intermediate | Structure | Name | Mass [M + H]⁺ |
|---|---|---|---|
| Intermediate 12 | 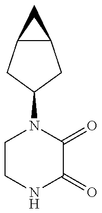 [SMILES: O=C1NCCN([C@H]2C[C@H]3C[C@H]3C2)C1=O] | 1-((cis)-bicyclo[3.1.0]hexan-3- yl)piperazine-2,3-dione | 195 |
| Intermediate 13 | 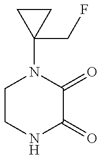 [SMILES: O=C1NCCN(C2(CF)CC2)C1=O] | 1-(1- (fluoromethyl)cyclopropyl)piperazine- 2,3-dione | 187 |
| Intermediate 14 | 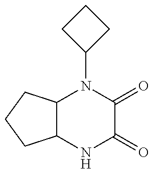 [SMILES: O=C1NC2CCCC2N(C2CCC2)C1=O] | 1-cyclobutylhexahydro-1H- cyclopenta[b]pyrazine-2,3-dione | 209 |

### Intermediate 15

### Preparation of 5-(bromomethyl)-2-phenylpyridine

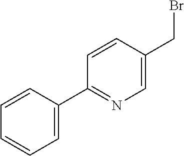 [SMILES: BrCc1ccc(-c2ccccc2)nc1]

### Step A: 5-Methyl-2-phenylpyridine

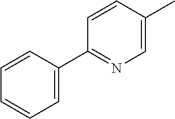

A mixture of 2-bromo-5-methylpyridine (2.00 g, 11.6 mmol), Pd(dppf)Cl₂ (0.43 g, 0.58 mmol), phenylboronic acid (1.56 g, 12.8 mmol), and K₃PO₄ (7.40 g, 34.9 mmol) in 1,4-dioxane (20 mL) and water (2 mL) was sparged with a stream of nitrogen at room temperature for 5 minutes. The reaction mixture was stirred and heated to 80° C. for 15 hours. The reaction mixture was cooled to room temperature, quenched with saturated aqueous ammonium chloride (30 mL), and extracted with EtOAc (3×30 mL). The organic layers were combined, washed with brine (15 mL), dried over anhydrous sodium sulfate, filtered, and concentrated under reduced pressure. The residue was purified by silica gel chromatography (eluting ethyl acetate in petroleum ether) to afford 5-methyl-2-phenylpyridine. ¹H NMR (400 MHz, chloroform-d) δ 8.51 (d, J=0.7 Hz, 1H), 7.99-7.91 (m, 2H), 7.65-7.59 (m, 1H), 7.57-7.51 (m, 1H), 7.49-7.42 (m, 2H), 7.40-7.33 (m, 1H), 2.36 (s, 3H). LC/MS (m/z): 170 (M+H)⁺

### Step B: 5-(bromomethyl)-2-phenylpyridine

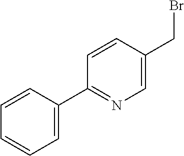 [SMILES: BrCc1ccc(-c2ccccc2)nc1]

A mixture of 5-methyl-2-phenylpyridine (400 mg, 2.36 mmol), N-bromosuccinimide (395 mg, 2.22 mmol), and azobisisobutyronitrile (78 mg, 0.47 mmol) in carbon tetrachloride (10 mL) was stirred and heated to 80° C. for 16 hours. The reaction mixture was cooled to room temperature and concentrated under reduced pressure. The residue was purified by silica gel chromatography (eluting ethyl acetate in petroleum ether) to afford 5-(bromomethyl)-2-phenylpyridine. LC/MS (m/z): 248, 250 (M+H)⁺

Intermediates shown in Intermediate Table 3 below, were prepared according to procedures analogous to those outlined in Intermediate 15 above using the appropriate starting materials, described in the Preparations or Intermediates above, or as obtained from commercial sources.

Intermediate Table 3

| Intermediate | Structure | Name | Mass [M + H]⁺ |
|---|---|---|---|
| Intermediate 16 | 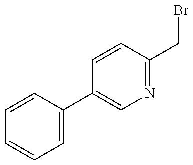 [SMILES: BrCc1ccc(-c2ccccc2)cn1] | 2-(bromomethyl)-5-phenylpyridine | 248, 250 |

### Intermediate 17

### Preparation of 5-(1-bromoethyl)-2-phenylpyridine

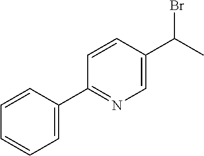

### Step A: 1-(6-phenylpyridin-3-yl)ethan-1-ol

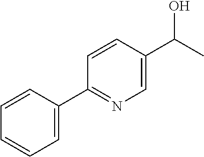

A mixture of 1-(6-bromopyridin-3-yl)ethanol (700 mg, 3.46 mmol), Pd(dppf)Cl₂ (76 mg, 0.10 mmol), phenylboronic acid (549 mg, 4.50 mmol), and K₃PO₄ (2206 mg, 10.39 mmol) in 1,4-dioxane (20 mL) and water (2 mL) at room temperature was sparged with a stream of nitrogen for 5 minutes. The reaction mixture was stirred and heated to 80° C. for 15 hours. The reaction mixture was cooled to room temperature, quenched with saturated aqueous ammonium chloride (30 mL), and extracted with EtOAc (3×30 mL). The organic layers were combined, washed with brine (15 mL), dried over anhydrous sodium sulfate, filtered, and concentrated under reduced pressure. The residue was purified by silica gel chromatography (eluting ethyl acetate in petroleum ether) to afford 1-(6-phenylpyridin-3-yl)ethanol. ¹H NMR (400 MHz, chloroform-d) δ 8.62 (d, J=2.2 Hz, 1H), 7.98-7.91 (m, 2H), 7.81-7.72 (m, 1H), 7.71-7.64 (m, 1H), 7.48-7.42 (m, 2H), 7.42-7.36 (m, 1H), 4.96 (q, J 6.6 Hz, 1H), 1.53 (d, J=6.6 Hz, 3H). LC/MS (m/z): 200 (M+H)⁺

### Step B: 5-(1-bromoethyl)-2-phenylpyridine

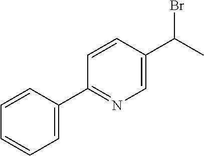

Carbon tetrabromide (374 mg, 1.13 mmol) was added to a mixture of 1-(6-phenylpyridin-3-yl)ethanol (150 mg, 0.753 mmol) and triphenylphosphine (355 mg, 1.36 mmol) in DCM (5 mL) at 0° C. under a nitrogen atmosphere. The reaction mixture was stirred at room temperature for 2 hours. The reaction mixture was filtered, and the filtrate was concentrated under reduced pressure. The residue was purified by silica gel chromatography (eluting ethyl acetate in petroleum ether) to afford 5-(1-bromoethyl)-2-phenylpyridine. ¹H NMR (400 MHz, chloroform-d) δ 8.70 (d, J=2.2 Hz, 1H), 7.97 (dd, J 1.2, 8.3 Hz, 2H), 7.85 (dd, J 2.4, 8.3 Hz, 1H), 7.72 (d, J=8.3 Hz, 1H), 7.53-7.40 (m, 3H), 5.25 (q, J 6.8 Hz, 1H), 2.09 (d, J=6.8 Hz, 3H). LC/MS (m/z): 262, 264 (M+H)⁺

Intermediates shown in Intermediate Table 4 below, were prepared according to procedures analogous to those outlined in Intermediate 17 above using the appropriate starting materials, described in the Preparations or Intermediates above, or as obtained from commercial sources.

Intermediate Table 4

| Intermediate | Structure | Name | Mass [M + H]⁺ |
|---|---|---|---|
| Intermediate 18 | 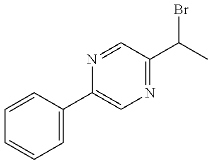 | 2-(1-bromoethyl)-5-phenylpyrazine | 263, 265 |
| Intermediate 19 | 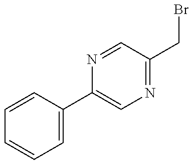 [SMILES: BrCc1cnc(-c2ccccc2)cn1] | 2-(bromomethyl)-5-phenylpyrazine | 249, 251 |

### Intermediate 20

### Preparation of 2-(4-cyclopentyl-2,3-dioxopiperazin-1-yl)acetic acid

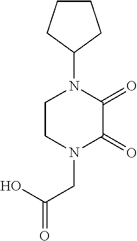 [SMILES: O=C(O)CN1CCN(C2CCCC2)C(=O)C1=O]

### Step A: tert-butyl 2-(4-cyclopentyl-2,3-dioxopiperazin-1-yl)acetate

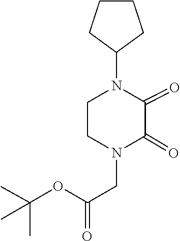 [SMILES: CC(C)(C)OC(=O)CN1CCN(C2CCCC2)C(=O)C1=O]

NaH (0.285 g, 7.13 mmol) was added to a mixture of 1-cyclopentylpiperazine-2,3-dione (1.00 g, 5.49 mmol) in DMF (15 ml) at 0° C. The reaction mixture was stirred at 0° C. for 15 minutes. tert-Butyl 2-bromoacetate (0.975 mL, 6.04 mmol) was added to the reaction mixture at 0° C. The reaction mixture was warmed to room temperature and stirred for 12 hours. The reaction mixture was quenched with water (60 mL) and extracted with EtOAc (3×60 mL). The organic layers were combined, washed with 10% aqueous LiCl (3×10 mL) and brine (5 mL), dried over anhydrous sodium sulfate, filtered, and concentrated under reduced pressure. The residue was purified by silica gel chromatography (eluting methanol in dichloromethane) to afford tert-butyl 2-(4-cyclopentyl-2,3-dioxopiperazin-1-yl)acetate. ¹H NMR (500 MHz, methanol-d₄) δ 4.84-4.75 (m, 1H), 4.15 (s, 2H), 3.66-3.56 (m, 4H), 1.96-1.86 (m, 2H), 1.83-1.74 (m, 2H), 1.70-1.58 (m, 4H), 1.48 (s, 9H). LC/MS (m/z): 297 (M+H)⁺

### Step B: 2-(4-cyclopentyl-2,3-dioxopiperazin-1-yl)acetic acid

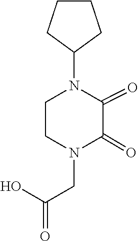 [SMILES: O=C(O)CN1CCN(C2CCCC2)C(=O)C1=O]

HCl (4.0 M in dioxane, 3.0 ml, 12 mmol) was added to a mixture of tert-butyl 2-(4-cyclopentyl-2,3-dioxopiperazin-1-yl)acetate (1.20 g, 4.05 mmol) in DCM (4 mL). The reaction mixture was stirred at room temperature for 3.5 hours. The reaction mixture was concentrated under reduced pressure to afford 2-(4-cyclopentyl-2,3-dioxopiperazin-1-yl)acetic acid, which was used without purification in the next step. ¹H NMR (500 MHz, methanol-d₄) δ=4.80 (quin, J=8.3 Hz, 1H), 4.23 (s, 2H), 3.67-3.58 (m, 4H), 1.96-1.86 (m, 2H), 1.83-1.72 (m, 2H), 1.72-1.58 (m, 4H). LC/MS (m/z): 241 (M+H)⁺

### Intermediate 21

### Preparation of 4-(bromomethyl)-3-fluoro-1,1′-biphenyl

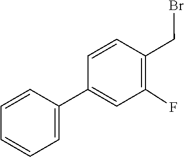 [SMILES: Fc1cc(-c2ccccc2)ccc1CBr]

### Step A: 3-fluoro-4-methyl-1,1′-biphenyl

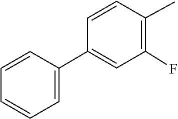

A mixture of (3-fluoro-4-methylphenyl)boronic acid (0.50 g, 3.3 mmol), bromobenzene (0.51 g, 3.3 mmol), potassium phosphate (0.90 g, 4.2 mmol) and PdCl₂(dppf) (0.19 g, 0.26 mmol) in 1,4-dioxane (10 mL) and water (2.0 mL) was sparged with nitrogen for 10 minutes at room temperature. The reaction mixture was stirred and heated to 60° C. for 2 hours. The reaction mixture was cooled to room temperature and diluted with EtOAc (50 mL) and water (40 mL). The organic layer was separated, dried over anhydrous sodium sulfate, filtered, and concentrated under reduced pressure. The residue was purified by silica gel chromatography (eluting ethyl acetate in petroleum ether) to afford 3-fluoro-4-methyl-1,1′-biphenyl. ¹H NMR (400 MHz, methanol-d₄) δ 7.61-7.52 (m, 2H), 7.40 (t, J=7.6 Hz, 2H), 7.35-7.22 (m, 4H), 2.27 (d, J=1.7 Hz, 3H).

### Step B: 4-(bromomethyl)-3-fluoro-1,1′-biphenyl

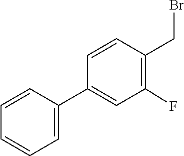 [SMILES: Fc1cc(-c2ccccc2)ccc1CBr]

A mixture of 3-fluoro-4-methyl-1,1′-biphenyl (500 mg, 2.68 mmol), N-bromosuccinimide (478 mg, 2.68 mmol), and benzoyl peroxide (65 mg, 0.27 mmol) in carbon tetrachloride (10 mL) was stirred and heated to 90° C. for 12 hours. The reaction mixture was cooled to room temperature and concentrated under reduced pressure. The residue was purified by silica gel chromatography (eluting ethyl acetate in petroleum ether) to afford 4-(bromomethyl)-3-fluoro-1,1′-biphenyl.

Intermediates shown in Intermediate Table 5 below, were prepared according to procedures analogous to those outlined in Intermediate 21 above using the appropriate starting materials, described in the Preparations or Intermediates above, or as obtained from commercial sources.

Intermediate Table 5

| Intermediate | Structure | Name | Mass [M + H]⁺ |
|---|---|---|---|
| Intermediate 22 | 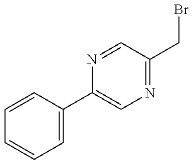 [SMILES: BrCc1cnc(-c2ccccc2)cn1] | 2-(bromomethyl)-5-phenylpyrimidine | 249, 251 |
| Intermediate 23 | 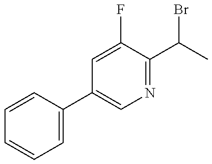 [SMILES: CC(Br)c1ncc(-c2ccccc2)cc1F] | 2-(bromomethyl)-3-fluoro-5- phenylpyridine | 266, 268 |
| Intermediate 24 | 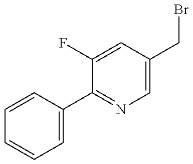 [SMILES: Fc1cc(CBr)cnc1-c1ccccc1] | 5-(bromomethyl)-3-fluoro-2- phenylpyridine | 266, 268 |
| Intermediate 25 | 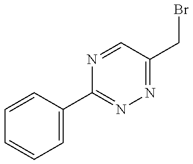 [SMILES: BrCc1cnc(-c2ccccc2)nn1] | 6-(bromomethyl)-3-phenyl-1,2,4- triazine | 250, 252 |
| Intermediate 26 | 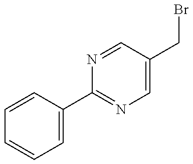 [SMILES: BrCc1cnc(-c2ccccc2)nc1] | 5-(bromomethyl)-2-phenylpyrimidine | 249, 251 |
| Intermediate 27 | 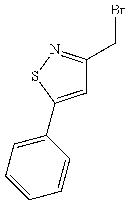 [SMILES: BrCc1cc(-c2ccccc2)sn1] | 3-(bromomethyl)-5-phenylisothiazole | 254, 256 |

### Intermediate 28

### Preparation of (3-fluoro-5-(thiazol-2-yl)pyridin-2-yl)methyl methanesulfonate

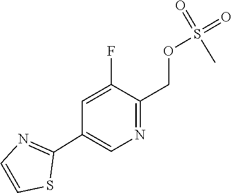 [SMILES: CS(=O)(=O)OCc1ncc(-c2nccs2)cc1F]

A mixture of PdCl₂(dppf) (18 mg, 0.024 mmol), (5-bromo-3-fluoropyridin-2-yl)methanol (50 mg, 0.24 mmol), and 2-(tributylstannyl)thiazole (0.099 mL, 0.32 mmol) in 1,4-dioxane (2 mL) was sparged with nitrogen for 1 minute at room temperature. The reaction mixture was stirred and heated at 90° C. for 30 minutes. The reaction mixture was cooled to room temperature. Triethylamine (0.135 mL, 0.971 mmol) and methanesulfonyl chloride (0.038 mL, 0.49 mmol) were added to the reaction mixture. The reaction mixture was stirred for 2 hours. The reaction mixture was diluted with DCM and filtered through Celite. The reaction mixture was concentrated under reduced pressure, and the residue was purified by basic alumina chromatography (eluting EtOAc:EtOH (3:1) in hexanes) to afford (3-fluoro-5-(thiazol-2-yl)pyridin-2-yl)methyl methanesulfonate. LC/MS (m/z): 289 (M+H)⁺

### Intermediate 29

### Preparation of methyl 2-(cyclopentyl(2-oxoethyl)amino)-2-oxoacetate

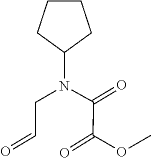

### Step A: methyl 2-(cyclopentylamino)-2-oxoacetate

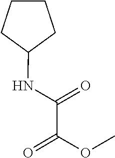

Hunig's base (4.35 mL, 25.0 mmol) was added to a mixture of cyclopentanamine (1.97 ml, 20.0 mmol) in DCM (100 mL) at 0° C. Methyl 2-chloro-2-oxoacetate (1.84 mL, 20.0 mmol) was added dropwise to the reaction mixture at 0° C. The reaction mixture was stirred for 5 hours and then was quenched with saturated aqueous sodium bicarbonate. The mixture was passed through a phase separator. The organic layer was concentrated under reduced pressure to afford methyl 2-(cyclopentylamino)-2-oxoacetate, which was used without purification in the next step. LC/MS (m/z): 172 (M+H)⁺

### Step B: methyl 2-(allyl(cyclopentyl)amino)-2-oxoacetate

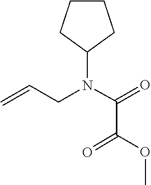

Sodium hydride (0.816 g, 20 mmol, 60% dispersion in mineral oil) was added to an oven-dried flask under a nitrogen atmosphere. DMF (60 mL) was added, and the mixture was placed in a water bath. A mixture of methyl 2-(cyclopentylamino)-2-oxoacetate (3.25 g, 19.0 mmol) in DMF (15.0 mL) was slowly added to the reaction mixture. The reaction mixture was cooled to 0° C. and allyl bromide (2.46 mL, 28.5 mmol) was added dropwise. The reaction mixture was warmed to room temperature and stirred for 18 hours. The reaction mixture was cooled to 0° C. and quenched with isopropanol (1 mL) followed by water (10 mL). The mixture was stirred for 30 minutes, then diluted with water (100 mL) and extracted with diethyl ether (3×100 mL). The organic layers were combined, washed with brine (25 mL), dried over magnesium sulfate, filtered, and concentrated under reduced pressure. The residue was purified by silica gel chromatography (eluting ethyl acetate in hexanes) to afford methyl 2-(allyl(cyclopentyl)amino)-2-oxoacetate. LC/MS (m/z): 212 (M+H)⁺

### Step C: methyl 2-(cyclopentyl(2-oxoethyl)amino)-2-oxoacetate

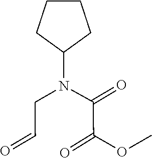

A mixture of methyl 2-(allyl(cyclopentyl)amino)-2-oxoacetate (0.300 g, 1.42 mmol) in DCM (25 mL) was cooled to −78° C. in an acetone/dry ice bath and stirred for 5 minutes. Ozone was bubbled through the reaction mixture for 10 minutes using the Triogen ozonolysis machine (connected to compressed air). When the reaction was complete (monitored by LCMS), air was bubbled through the reaction mixture. Dimethyl sulfide (1.58 ml, 21.3 mmol) was added to the reaction mixture, and the mixture was stirred at room temperature for 18 hours. The mixture was concentrated under reduced pressure to afford methyl 2-(cyclopentyl(2-oxoethyl)amino)-2-oxoacetate, which was used without purification in the next step. LC/MS (m/z): 214 (M+H)⁺.

Intermediates, as shown in Intermediate Table 6 below, were or may be prepared according to procedures analogous to those outlined in Intermediate 29 above using the appropriate starting materials, described in the Preparations or Intermediates above, or as obtained from commercial sources.

Intermediate Table 6

| Intermediate | Structure | Name | Mass [M + H]⁺ |
|---|---|---|---|
| Intermediate 30 | 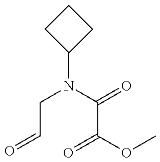 | methyl 2-(cyclobutyl(2- oxoethyl)amino)-2-oxoacetate | 200 |
| Intermediate 31 | 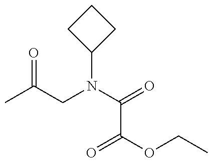 [SMILES: CCOC(=O)C(=O)N(CC(C)=O)C1CCC1] | ethyl 2-(cyclobutyl(2- oxopropyl)amino)-2-oxoacetate | 228 |
| Intermediate 32 | 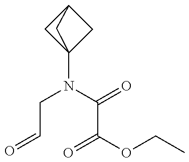 [SMILES: CCOC(=O)C(=O)N(CC=O)C12CC(C1)C2] | ethyl 2-(bicyclo[1.1.1]pentan-1- yl(2-oxoethyl)amino)-2-oxoacetate | 226 |
| Intermediate 33 | 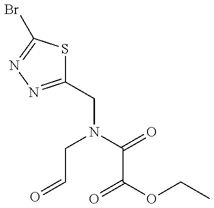 [SMILES: CCOC(=O)C(=O)N(CC=O)Cc1nnc(Br)s1] | ethyl 2-(((5-bromo-1,3,4-thiadiazol- 2-yl)methyl)(2-oxoethyl)amino)-2- oxoacetate | 336, 338 |

### Intermediate 34

### Preparation of (S)-1-(5-phenyl-1,3,4-thiadiazol-2-yl)ethan-1-amine

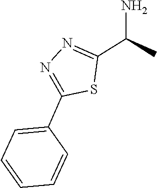

### Step A: tert-butyl (S)-(1-(2-benzoylhydrazineyl)-1-oxopropan-2-yl)carbamate

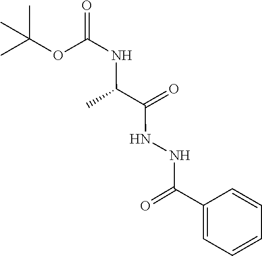 [SMILES: C[C@H](NC(=O)OC(C)(C)C)C(=O)NNC(=O)c1ccccc1]

Hunig's base (3.58 mL, 20.5 mmol) and 1-propanephosphonic anhydride (50% in EtOAc, 6.25 mL, 10.5 mmoL) were added to a mixture of benzohydrazide (1.36 g, 10 mmol) and (tert-butoxycarbonyl)-L-alanine (1.9 g, 10 mmol) in DCM (100 mL) at room temperature. The reaction mixture was stirred at room temperature for 18 hours. The reaction mixture was diluted with water (50 mL), and the mixture was passed through a phase separator. The organic layer was dried with magnesium sulfate, filtered, and concentrated under reduced pressure to afford tert-butyl (S)-(1-(2-benzoylhydrazineyl)-1-oxopropan-2-yl)carbamate. LC/MS (m/z): 330 (M+Na)⁺ Step B: (S)-1-(5-phenyl-1,3,4-thiadiazol-2-yl)ethan-1-amine

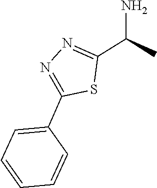

THE (50 mL) was added to a mixture of tert-butyl (S)-(1-(2-benzoylhydrazineyl)-1-oxopropan-2-yl)carbamate (2.9 g, 9.5 mmol) and Lawessons reagent (7.68 g, 19.0 mmol) under a nitrogen atmosphere. The reaction mixture was stirred and heated to 65° C. under a reflux condenser for 15 hours. The reaction mixture was cooled to room temperature and concentrated under reduced pressure. The residue was purified by silica gel chromatography (eluting ethyl acetate in hexanes) to afford the Boc-protected thiodiazole intermediate. The isolated intermediate was diluted with DCM (50 mL) and TFA (3.66 mL, 47.5 mmol). The mixture was stirred at room temperature for 2 hours. The mixture was concentrated under reduced pressure to afford (S)-1-(5-phenyl-1,3,4-thiadiazol-2-yl)ethan-1-amine, which was used without purification in the next step. LC/MS (m/z): 206 (M+H)⁺

Intermediates shown in Intermediate Table 7 below, were or may be prepared according to procedures analogous to those outlined in Intermediate 34 above using the appropriate starting materials, described in the Preparations or Intermediates above, or as obtained from commercial sources.

Intermediate Table 7

| Intermediate | Structure | Name | Mass [M + H]⁺ |
|---|---|---|---|
| Intermediate 35 | 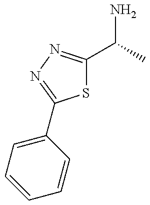 | (R)-1-(5-phenyl-1,3,4-thiadiazol-2- yl)ethan-1-amine | 206 |
| Intermediate 36 | 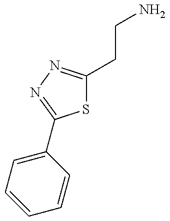 [SMILES: NCCc1nnc(-c2ccccc2)s1] | 2-(5-phenyl-1,3,4-thiadiazol-2- yl)ethan-1-amine | 206 |
| Intermediate 37 | 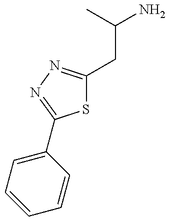 | 1-(5-phenyl-1,3,4-thiadiazol-2- yl)propan-2-amine | 220 |
| Intermediate 38 | 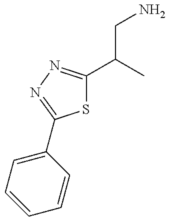 | 2-(5-phenyl-1,3,4-thiadiazol-2- yl)propan-1-amine | 220 |

### Intermediate 39

### Preparation of 2-(chloromethyl)-5-phenylpyrimidine

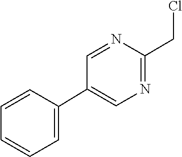 [SMILES: ClCc1ncc(-c2ccccc2)cn1]

### Step A: (5-phenylpyrimidin-2-yl)methanol

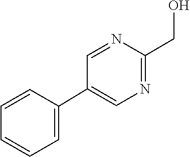 [SMILES: OCc1ncc(-c2ccccc2)cn1]

A mixture of (5-bromopyrimidin-2-yl)methanol (940 mg, 4.97 mmol), phenylboronic acid (667 mg, 5.47 mmol), Pd(dppf)Cl₂—CH₂C₁₂ adduct (203 mg, 0.249 mmol), and potassium carbonate (1380 mg, 9.95 mmol) in 1,4-dioxane (10 mL) and water (1 mL) was sparged with argon for 5 minutes at room temperature. The reaction mixture was heated to 60° C. and stirred under an argon atmosphere for 4 hours. The reaction mixture was cooled to room temperature and diluted with ethyl acetate (25 mL). Magnesium sulfate (˜5 g) was added, and the mixture was stirred at room temperature for 10 minutes. The mixture was filtered through CELITE, while washing with ethyl acetate. The filtrate was concentrated under reduced pressure, and the residue was purified by silica gel chromatography (eluting ethyl acetate in hexanes) to afford (5-phenylpyrimidin-2-yl)methanol. LC/MS (m/z): 187 (M+H)⁺

### Step B: 2-(chloromethyl)-5-phenylpyrimidine

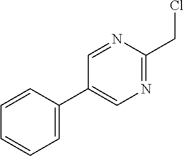 [SMILES: ClCc1ncc(-c2ccccc2)cn1]

Methanesulfonyl chloride (0.500 mL, 6.42 mmol) was added to a mixture of (5-phenylpyrimidin-2-yl)methanol (920 mg, 4.94 mmol) and triethylamine (1.38 mL, 9.88 mmol) in dichloromethane (10 mL) at 0° C. The reaction mixture was warmed to room temperature and stirred for 18 hours. The reaction mixture concentrated under reduced pressure, and the residue was purified by silica gel chromatography (eluting ethyl acetate in hexanes) to afford 2-(chloromethyl)-5-phenylpyrimidine. ¹H NMR (500 MHz, DMSO-d₆) δ 9.19 (s, 2H), 7.87-7.82 (m, 2H), 7.56 (t, J=7.4 Hz, 2H), 7.50 (t, J=7.3 Hz, 1H), 4.88 (s, 2H). LC/MS (m/z): 205 (M+H)⁺

### Intermediate 40

### Preparation of 1-cyclobutyl-1,4-dihydropyrazine-2,3-dione

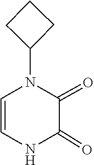 [SMILES: O=c1[nH]ccn(C2CCC2)c1=O]

### Step A: ethyl 2-(cyclobutylamino)-2-oxoacetate

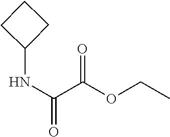

Ethyl 2-chloro-2-oxoacetate (0.79 mL, 7.0 mmol) was added to a mixture of cyclobutanamine (0.60 mL, 7.0 mmol) and triethylamine (0.98 mL, 7.0 mmol) in dichloromethane (20 mL) at 0° C. The reaction mixture was stirred at 0° C. for 15 minutes. The reaction mixture was partially concentrated under reduced pressure (˜½ volume) and then directly purified by silica gel chromatography (eluting ethyl acetate in hexanes) to afford ethyl 2-(cyclobutylamino)-2-oxoacetate. LC/MS (m/z): 172 (M+H)⁺

### Step B: N¹-cyclobutyl-N²-(2,2-dimethoxyethyl)oxalamide

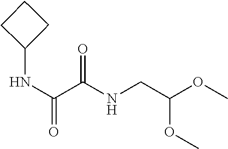 [SMILES: COC(CNC(=O)C(=O)NC1CCC1)OC]

A mixture of ethyl 2-(cyclobutylamino)-2-oxoacetate (1.2 g, 7.0 mmol) and 2,2-dimethoxyethan-1-amine (1.53 mL, 14.0 mmol) in 2-propanol (10 mL) was stirred at 20° C. for 18 hours to afford a suspension of solids. The mixture was filtered, and the isolated solids were washed with additional 2-propanol (5×2 mL) and then dried under high vacuum to afford N¹-cyclobutyl-N²-(2,2-dimethoxyethyl)oxalamide. ¹H NMR (500 MHz, DMSO-d₆) δ 9.00 (d, J=8.3 Hz, 1H), 8.59 (t, J=6.0 Hz, 1H), 4.49 (t, J=5.5 Hz, 1H), 4.28-4.20 (m, 1H), 3.25-3.20 (m, 8H), 2.17-2.09 (m, 4H), 1.67-1.58 (m, 2H). LC/MS (m/z): 253 (M+Na)⁺

### Step C: 1-cyclobutyl-1,4-dihydropyrazine-2,3-dione

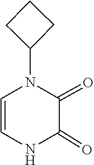 [SMILES: O=c1[nH]ccn(C2CCC2)c1=O]

A mixture of N¹-cyclobutyl-N²-(2,2-dimethoxyethyl)oxalamide (1.2 g, 5.2 mmol) and HCl (37% in water, 0.043 mL, 0.52 mmol) in acetic acid (3.0 mL) was heated to 100° C. and stirred for 1 hour. The reaction mixture was cooled to room temperature and concentrated under reduced pressure. The residue was purified by silica gel chromatography (eluting [1:3 ethanol/ethyl acetate] in dichloromethane) to afford 1-cyclobutyl-1,4-dihydropyrazine-2,3-dione. ¹H NMR (500 MHz, DMSO-d₆) δ 11.22 (s, 1H), 6.65 (d, J=6.0 Hz, 1H), 6.35 (d, J=6.0 Hz, 1H), 4.89 (p, J=8.9 Hz, 1H), 2.28-2.21 (m, 4H), 1.78-1.68 (m, 2H). LC/MS (m/z): 167 (M+H)⁺

Intermediates shown in Intermediate Table 8 below, were prepared according to procedures analogous to those outlined in Intermediate 40 above using the appropriate starting materials, described in the Preparations or Intermediates above, or as obtained from commercial sources.

Intermediate Table 8

| Intermediate | Structure | Name | Mass [M + H]⁺ |
|---|---|---|---|
| Intermediate 41 | 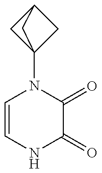 [SMILES: O=c1[nH]ccn(C23CC(C2)C3)c1=O] | 1-(bicyclo[1.1.1]pentan-1-yl)-1,4- dihydropyrazine-2,3-dione | 179 |
| Intermediate 42 | 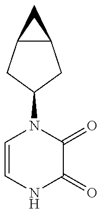 [SMILES: O=c1[nH]ccn([C@H]2C[C@H]3C[C@H]3C2)c1=O] | 1-(cis)-bicyclo[3.1.0]hexan-3-yl)-1,4- dihydropyrazine-2,3-dione | 193 |
| Intermediate 43 | 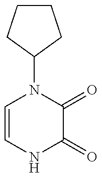 [SMILES: O=c1[nH]ccn(C2CCCC2)c1=O] | 1-cyclopentyl-1,4-dihydropyrazine- 2,3-dione | 181 |
| Intermediate 44 | 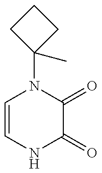 | 1-(1-methylcyclobutyl)-1,4- dihydropyrazine-2,3-dione | 181 |
| Intermediate 45 | 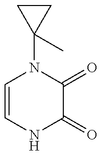 | 1-(1-methylcyclopropyl)-1,4- dihydropyrazine-2,3-dione | 167 |
| Intermediate 46 | 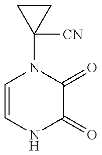 [SMILES: N#CC1(n2cc[nH]c(=O)c2=O)CC1] | 1-(2,3-dioxo-3,4-dihydropyrazin- 1(2H)-yl)cyclopropane-1-carbonitrile | 178 |
| Intermediate 47 | 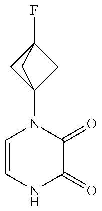 [SMILES: O=c1[nH]ccn(C23CC(F)(C2)C3)c1=O] | 1-(3-fluorobicyclo[1.1.1]pentan-1- yl)-1,4-dihydropyrazine-2,3-dione | 197 |
| Intermediate 48 | 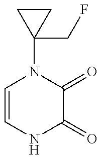 [SMILES: O=c1[nH]ccn(C2(CF)CC2)c1=O] | 1-(1-(fluoromethyl)cyclopropyl)-1,4- dihydropyrazine-2,3-dione | 185 |

### Intermediate 49

### Preparation of 6-methyl-3-phenyl-1,2,4-triazine

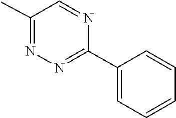

### Step A: N-(prop-2-yn-1-yl)benzamide

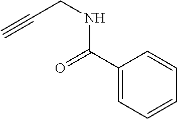

A solution of benzoyl chloride (2.46 mL, 21.3 mmol) in DCM (12 mL) was added dropwise to a mixture of propargylamine (1.57 mL, 24.5 mmol) and N,N-diethylethanamine (5.95 mL, 42.7 mmol) in DCM (48 mL) at 0° C. The mixture was stirred at room temperature for 2 hours. The reaction mixture was quenched with HCl (1 N, 100 mL) and extracted with DCM (3×100 mL). The organic layers were combined, washed with brine (200 mL), dried over anhydrous Na₂SO₄, filtered, and concentrated under reduced pressure. The residue was purified by silica gel chromatography (eluting ethyl acetate in petroleum ether) to afford N-(prop-2-yn-1-yl)benzamide. ¹H NMR (500 MHz, chloroform-d) δ 7.79 (d, J=7.3 Hz, 2H), 7.54-7.49 (m, 1H), 7.47-7.41 (m, 2H), 6.33 (br s, 1H), 4.26 (dd, J=2.4, 5.2 Hz, 2H), 2.29 (t, J=2.4 Hz, 1H). LC/MS (m/z): 160 (M+H)⁺

### Step B: 6-methyl-3-phenyl-1,2,4-triazine

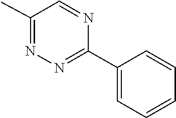

A mixture of N-(prop-2-yn-1-yl)benzamide (1.0 g, 6.3 mmol), BOC-hydrazine (644 μL, 6.28 mmol), and zinc trifluoromethanesulfonate (Zn(OTf)₂) (685 mg, 1.89 mmol) was stirred and heated at 120° C. for 4 hours under an inert atmosphere. The mixture was filtered, and the filtrate was concentrated under reduced pressure. The residue was dissolved in toluene (80 mL) and then added to a mixture of potassium ferricyanide (K₃[Fe(CN)₆]) (3.10 g, 9.42 mmol) and NaOH (628 mg, 15.7 mmol) in water (50 mL) at room temperature. The biphasic mixture was vigorously stirred at room temperature for 12 hours. The organic phase was separated, dried over anhydrous Na₂SO₄, filtered, and concentrated under reduced pressure. The residue was purified by silica gel chromatography (eluting ethyl acetate in petroleum ether) to afford 6-methyl-3-phenyl-1,2,4-triazine. ¹H NMR (400 MHz, chloroform-d) δ 8.54 (s, 1H), 8.53-8.48 (m, 2H), 7.56-7.52 (m, 3H), 2.76 (s, 3H). LC/MS (m/z): 172 (M+H)⁺

### Intermediate 50

### Preparation of 3-(bromomethyl)-5-phenylisoxazole

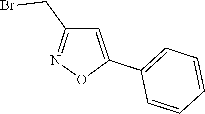 [SMILES: BrCc1cc(-c2ccccc2)on1]

### Step A: (5-phenylisoxazol-3-yl)methanol

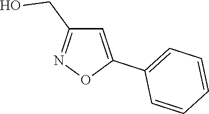 [SMILES: OCc1cc(-c2ccccc2)on1]

BH₃ dimethylsulfide (0.529 mL, 5.3 mmol, 10 M in THF) was added to a mixture of 5-phenylisoxazole-3-carboxylic acid (500 mg, 2.64 mmol) in THE (10 mL) at room temperature. The reaction mixture was stirred and heated to 60° C. for 4 hours. The reaction mixture was quenched with MeOH and concentrated under reduced pressure to afford (5-phenylisoxazol-3-yl)methanol which was used without purification. LC/MS (m/z): 176 (M+H)⁺

### Step B: 3-(bromomethyl)-5-phenylisoxazole

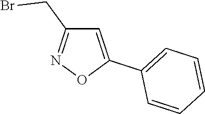 [SMILES: BrCc1cc(-c2ccccc2)on1]

(5-Phenylisoxazol-3-yl)methanol (400 mg, 2.28 mmol) was added to a mixture of triphenylphosphine (898 mg, 3.42 mmol) and carbon tetrabromide (1140 mg, 3.42 mmol) in DCM (10 mL) at room temperature. The reaction mixture was stirred at room temperature for 4 hours. The reaction mixture was concentrated under reduced pressure. The residue was purified by silica gel chromatography (eluting ethyl acetate in petroleum ether) to afford 3-(bromomethyl)-5-phenylisoxazole. ¹H NMR (500 MHz, methanol-d₄) δ 7.86-7.82 (m, 2H), 7.58-7.38 (m, 3H), 6.90 (s, 1H), 4.56 (s, 2H). LC/MS (m/z): 238, 240 (M+H)⁺

### Intermediate 51

### Preparation of 2-(1-bromoethyl)-5-phenylpyrimidine

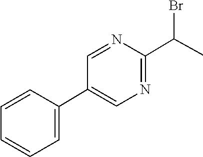

### Step A: 1-(5-phenylpyrimidin-2-yl)ethan-1-one

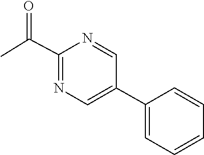

A mixture of 1-(5-bromopyrimidin-2-yl)ethanone (1.0 g, 5.0 mmol), phenylboronic acid (0.73 g, 6.0 mmol), K₃PO₄ (2.1 g, 10 mmol), and Pd(dppf)Cl₂ (0.36 g, 0.50 mmol) in 1,4-dioxane (10 mL) and water (2 mL) was sparged with N₂ at room temperature. The reaction mixture was then stirred and heated at 80° C. for 16 hours. The reaction mixture was cooled to room temperature, diluted with water (10 mL) and extracted with EtOAc (3×15 mL). The organic layers were combined, washed with brine (15 mL), filtered, and concentrated under reduced pressure. The residue was purified by silica gel chromatography (eluting ethyl acetate in petroleum ether) to afford 1-(5-phenylpyrimidin-2-yl)ethan-1-one. ¹H NMR (400 MHz, chloroform-d) δ 9.20-9.01 (m, 2H), 7.68-7.62 (m, 2H), 7.60-7.50 (m, 3H), 2.83 (s, 3H). LC/MS (m/z): 199 (M+H)⁺

### Step B: 1-(5-phenylpyrimidin-2-yl)ethan-1-ol

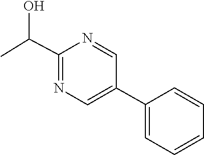

NaBH₄ (115 mg, 3.03 mmol) was added to a mixture of 1-(5-phenylpyrimidin-2-yl)ethan-1-one (500 mg, 2.52 mmol) in MeOH (25 mL) at 0° C. The reaction mixture was stirred at room temperature for 1 hour. The mixture was concentrated under reduced pressure. The residue was partitioned between water (20 mL) and EtOAc (20 mL). The organic layer was separated, and the aqueous layer was washed with additional EtOAc (3×20 mL). The organic layers were combined, washed with brine (20 mL), dried over anhydrous Na₂SO₄, filtered, and concentrated under reduced pressure. The residue was purified by silica gel chromatography (eluting ethyl acetate in petroleum ether) to afford 1-(5-phenylpyrimidin-2-yl)ethan-1-ol. ¹H NMR (400 MHz, methanol-d₄) δ 9.03 (s, 2H), 7.72-7.70 (m, 2H), 7.57-7.45 (m, 3H), 5.00-4.96 (m, 1H), 1.57-1.55 (m, 3H). LC/MS (m/z): 201 (M+H)⁺

### Step C: 2-(1-bromoethyl)-5-phenylpyrimidine

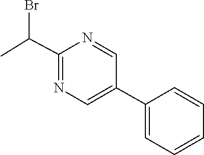

1-(5-Phenylpyrimidin-2-yl)ethan-1-ol (300 mg, 1.50 mmol) was added to a mixture of triphenylphosphine (589 mg, 2.25 mmol) and carbon tetrabromide (745 mg, 2.25 mmol) in DCM (10 mL) at room temperature. The mixture was stirred at room temperature for 16 hours. The reaction mixture was concentrated under reduced pressure. The residue was purified by silica gel chromatography (eluting ethyl acetate in petroleum ether) to afford 2-(1-bromoethyl)-5-phenylpyrimidine. ¹H NMR (500 MHz, methanol-d₄) δ 9.04 (s, 2H), 7.76-7.70 (m, 2H), 7.59-7.45 (m, 3H), 5.38-5.34 (m, 1H), 2.11-2.10 (m, 3H). LC/MS (m/z): 263, 265 (M+H)⁺

### Intermediate 52

### Preparation of 5-(bromomethyl)-2-(1H-1,2,3-triazol-1-yl)pyridine

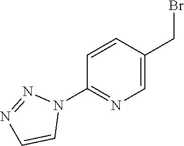 [SMILES: BrCc1ccc(-n2ccnn2)nc1]

### Step A: 5-methyl-2-(1H-1,2,3-triazol-1-yl)pyridine

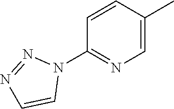

A mixture of 2H-1,2,3-triazole (1.24 g, 18.0 mmol), 2-fluoro-5-methylpyridine (1.00 g, 9.00 mmol), Cs₂CO₃ (8.80 g, 27.0 mmol) in DMF (30 mL) was stirred and heated at 70° C. for 2 hours. The mixture was cooled to room temperature and diluted with water (150 mL) and then extracted with EtOAc (200 mL). The organic layer was separated, dried over sodium sulfate, filtered, and concentrated under reduced pressure. The residue was purified by silica gel chromatography (eluting ethyl acetate in petroleum ether) to afford a mixture of 5-methyl-2-(1H-1,2,3-triazol-1-yl)pyridine as the first eluting peak and 5-methyl-2-(2H-1,2,3-triazol-2-yl)pyridine as the second eluting peak.

Peak 1: ¹H NMR (500 MHz, methanol-d₄) δ 8.75 (d, J=1.2 Hz, 1H), 8.41 (br s, 1H), 8.09-8.04 (m, 1H), 7.92-7.88 (m, 2H), 2.45 (s, 3H). LC/MS (m/z): 161 (M+H)⁺

Peak 2: ¹H NMR (500 MHz, methanol-d₄) δ 8.38 (s, 1H), 8.05-7.98 (m, 3H), 7.90 (br d, J=8.4 Hz, 1H), 2.45 (s, 3H). LC/MS (m/z): 161 (M+H)⁺

### Step B: 5-(bromomethyl)-2-(1H-1,2,3-triazol-1-yl)pyridine

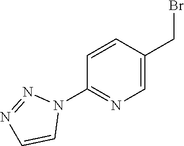 [SMILES: BrCc1ccc(-n2ccnn2)nc1]

A mixture of 5-methyl-2-(1H-1,2,3-triazol-1-yl)pyridine (90 mg, 0.56 mmol), N-bromosuccinimide (0.100 g, 0.56 mmol) and benzoyl peroxide (BPO) (14 mg, 0.056 mmol) in carbon tetrachloride (5 mL) was stirred and heated at 90° C. for 12 hours. The mixture was cooled to room temperature and concentrated under reduced pressure. The residue was purified by silica gel chromatography (eluting ethyl acetate in petroleum ether) to afford 5-(bromomethyl)-2-(1H-1,2,3-triazol-1-yl)pyridine. LC/MS (m/z): 239, 241 (M+H)⁺

### Intermediate 53

### Preparation of 1-(bicyclo[1.1.1]pentan-1-yl)piperazine-2,3-dione

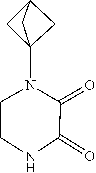 [SMILES: O=C1NCCN(C23CC(C2)C3)C1=O]

### Step A: ethyl 2-(bicyclo[1.1.1]pentan-1-ylamino)-2-oxoacetate

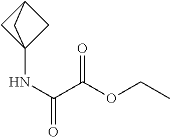

Ethyl 2-chloro-2-oxoacetate (1.87 mL, 16.7 mmol) was added to a mixture of bicyclo[1.1.1]pentan-1-amine hydrochloride (2.00 g, 16.7 mmol) and triethylamine (5.83 mL, 41.8 mmol) in dichloromethane (50 mL) at 0° C. The mixture was stirred at 0° C. for 15 minutes. The mixture was partially concentrated under reduced pressure (˜¼ volume) and filtered to remove triethylammonium chloride. The filtrate was directly purified by silica gel chromatography (eluting ethyl acetate in hexanes) to afford ethyl 2-(bicyclo[1.1.1]pentan-1-ylamino)-2-oxoacetate. LC/MS (m/z): 184 (M+H)⁺

### Step B: N¹-(bicyclo[1.1.1]pentan-1-yl)-N²-(2,2-dimethoxyethyl)oxalamide

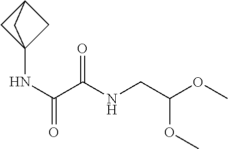 [SMILES: COC(CNC(=O)C(=O)NC12CC(C1)C2)OC]

A mixture of ethyl 2-(bicyclo[1.1.1]pentan-1-ylamino)-2-oxoacetate (3.08 g, 16.8 mmol) and 2,2-dimethoxyethan-1-amine (1.83 mL, 16.8 mmol) in 2-propanol (20 mL) was stirred at room temperature for 2 days. The mixture was concentrated under reduced pressure, and the isolated solids were dried under vacuum to afford N¹-(bicyclo[1.1.1]pentan-1-yl)-N²-(2,2-dimethoxyethyl)oxalamide. ¹H NMR (500 MHz, DMSO-d₆) δ 9.27 (s, 1H), 8.56 (t, J=5.9 Hz, 1H), 4.48 (t, J=5.5 Hz, 1H), 3.25 (s, 6H), 3.25-3.22 (m, 2H), 2.43 (s, 1H), 2.03 (s, 6H). LC/MS (m/z): 265 (M+Na)⁺

### Step C: 1-(bicyclo[1.1.1]pentan-1-yl)-1,4-dihydropyrazine-2,3-dione

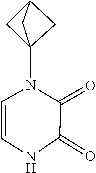 [SMILES: O=c1[nH]ccn(C23CC(C2)C3)c1=O]

A mixture of N¹-(bicyclo[1.1.1]pentan-1-yl)-N²-(2,2-dimethoxyethyl)oxalamide (3.33 g, 13.7 mmol) and HCl (37% in water, 0.056 mL, 0.69 mmol) in acetic acid (10.0 mL) was heated to 100° C. and stirred for 1 hour. The reaction mixture was cooled to room temperature and concentrated under reduced pressure. The residue was purified by silica gel chromatography (eluting [1:3 ethanol/ethyl acetate] in dichloromethane) to afford 1-(bicyclo[1.1.1]pentan-1-yl)-1,4-dihydropyrazine-2,3-dione. ¹H NMR (500 MHz, DMSO-d₆) δ 11.21 (s, 1H), 6.33 (d, J=5.9 Hz, 1H), 6.28 (d, J=5.9 Hz, 1H), 2.61 (s, 1H), 2.23 (s, 7H). LC/MS (m/z): 179 (M+H)⁺

### Step D: 1-(bicyclo[1.1.1]pentan-1-yl)piperazine-2,3-dione

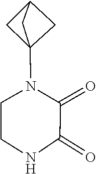 [SMILES: O=C1NCCN(C23CC(C2)C3)C1=O]

A mixture of 1-(bicyclo[1.1.1]pentan-1-yl)-1,4-dihydropyrazine-2,3-dione (5.00 g, 28.1 mmol), Pd(OH)₂ (20% w/w, 0.394 g, 0.561 mmol), and palladium on carbon (Pd/C) (10% w/w, 1.792 g, 1.7 mmol) was degassed with nitrogen. MeOH (200 mL) was added, and the mixture was degassed and backfilled with H₂ (three times). The mixture was stirred under a H₂ atmosphere (Pressure: 50 psi) and heated at 60° C. for 24 hours. The mixture was filtered, and the filtrate was concentrated under reduced pressure. The residue was purified by SFC (column Phenomenex-Cellulose-2 (250 mm*50 mm, 10 um); eluting 45% EtOH in C₀₂ with 0.1% ammonia) to afford 1-(bicyclo[1.1.1]pentan-1-yl)piperazine-2,3-dione. ¹H NMR (400 MHz, DMSO-d₆) δ 8.59 (br s, 1H), 3.50-3.40 (m, 2H), 3.36-3.31 (m, 2H), 2.50 (s, 1H), 2.12 (s, 6H). LC/MS (m/z): 181 (M+H)⁺

### Intermediate 54

### Preparation of 5-(bromomethyl)-2-(2H-1,2,3-triazol-2-yl)pyridine

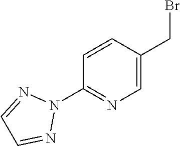 [SMILES: BrCc1ccc(-n2nccn2)nc1]

A mixture of 5-methyl-2-(2H-1,2,3-triazol-2-yl)pyridine (100 mg, 0.624 mmol), N-bromosuccinimide (111 mg, 0.624 mmol), and benzoyl peroxide (15 mg, 0.062 mmol) in chloroform (12 mL) was stirred and heated at 80° C. for 12 hours. The mixture was cooled to room temperature and concentrated under reduced pressure. The residue was purified by silica gel chromatography (eluting ethyl acetate in petroleum ether) to afford 5-(bromomethyl)-2-(2H-1,2,3-triazol-2-yl)pyridine. LC/MS (m/z): 239, 241 (M+H)⁺

### Intermediate 55

### Preparation of 3-(2-bromoethyl)-5-phenylisoxazole

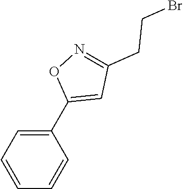 [SMILES: BrCCc1cc(-c2ccccc2)on1]

### Step A: methyl 3,5-dioxo-5-phenylpentanoate

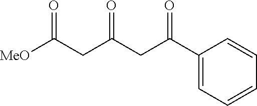 [SMILES: COC(=O)CC(=O)CC(=O)c1ccccc1]

Lithium diisopropylamide (LDA) in (2.0 M in THF, 27.5 mL, 55 mmol) and tetramethylethylenediamine (3.33 mL, 22.0 mmol) were added to a mixture of methyl 3-oxobutanoate (3.2 g, 28 mmol) in THF (50 mL) at −78° C. After stirring for 30 minutes at −78° C., methyl benzoate (3.0 g, 22 mmol) was added to the mixture at −78° C. The mixture was stirred at 0° C. for an additional 12 hours. The reaction mixture was quenched with 6 N HCl (100 mL) and extracted with EtOAc (3×100 mL). The organic layers were combined, washed with brine (200 mL), dried over anhydrous Na₂SO₄, filtered, and concentrated under reduced pressure. The residue was purified by silica gel chromatography (eluting ethyl acetate in petroleum ether) to afford methyl 3,5-dioxo-5-phenylpentanoate. ¹H NMR (500 MHz, chloroform-d) δ 7.91-7.86 (m, 2H), 7.57-7.52 (m, 1H), 7.49-7.43 (m, 2H), 7.38-7.30 (m, 1H), 6.29 (s, 1H), 3.77 (s, 3H), 3.50 (s, 2H). LC/MS (m/z): 221 (M+H)⁺

### Step B: ethyl 2-(5-phenylisoxazol-3-yl)acetate

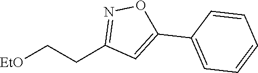 [SMILES: CCOCCc1cc(-c2ccccc2)on1]

A mixture of methyl 3,5-dioxo-5-phenylpentanoate (0.100 g, 0.454 mmol) and hydroxylamine hydrochloride (95 mg, 1.4 mmol) in EtOH (3 mL) was stirred and heated at 80° C. for 2 hours. The mixture was cooled to room temperature and concentrated under reduced pressure. The residue was partitioned between water (10 mL) and EtOAc (10 mL). The organic layer was separated, and the aqueous layer was re-extracted with EtOAc (3×10 mL). The organic layers were combined, washed with brine (20 mL), dried over anhydrous Na₂SO₄, filtered, and concentrated under reduced pressure. The residue was purified by reverse phase HPLC (eluting acetonitrile in water, with ammonium bicarbonate modifier) to afford ethyl 2-(5-phenylisoxazol-3-yl)acetate. ¹H NMR (400 MHz, chloroform-d) δ 7.84-7.75 (m, 2H), 7.50-7.40 (m, 3H), 6.62 (s, 1H), 4.23 (q, J=7.0 Hz, 2H), 3.79 (s, 2H), 1.30 (t, J=7.0 Hz, 3H). LC/MS (m/z): 232 (M+H)⁺

### Step C: 2-(5-phenylisoxazol-3-yl)ethan-1-ol

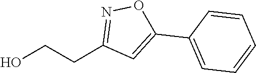 [SMILES: OCCc1cc(-c2ccccc2)on1]

Lithium borohydride (141 mg, 6.49 mmol) was added slowly to a mixture of ethyl 2-(5-phenylisoxazol-3-yl)acetate (0.500 g, 2.16 mmol) in THE (15 mL) at 0° C. The reaction mixture was stirred at 20° C. for 12 hours. The reaction mixture was quenched with saturated NH₄Cl aqueous solution (50 mL) and extracted with DCM (3×50 mL). The organic layers were combined, washed with brine (50 mL), dried over anhydrous Na₂SO₄, filtered, and concentrated under reduced pressure. The residue was purified by silica gel chromatography (eluting ethyl acetate in petroleum ether) to afford 2-(5-phenylisoxazol-3-yl)ethan-1-ol. LC/MS (m/z): 190 (M+H)⁺

### Step D: 3-(2-bromoethyl)-5-phenylisoxazole

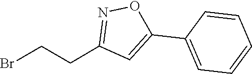 [SMILES: BrCCc1cc(-c2ccccc2)on1]

2-(5-phenylisoxazol-3-yl)ethan-1-ol (0.100 g, 0.529 mmol) was added to a mixture of triphenylphosphine (208 mg, 0.793 mmol) and carbon tetrabromide (263 mg, 0.793 mmol) in DCM (10 mL) at room temperature. The mixture was stirred at room temperature for 16 hours under a nitrogen atmosphere. The mixture was filtered, and the filtrate was concentrated under reduced pressure. The residue was purified by silica gel chromatography (eluting ethyl acetate in petroleum ether) to afford 3-(2-bromoethyl)-5-phenylisoxazole. ¹H NMR (400 MHz, methanol-d₄) δ 7.82 (dd, J 1.6, 7.9 Hz, 2H), 7.53-7.45 (m, 3H), 6.79 (s, 1H), 3.75 (t, J 7.0 Hz, 2H), 3.30-3.27 (m, 2H). LC/MS (m/z): 252, 254 (M+H)⁺

### Intermediate 56

### Preparation of (5-phenyl-1,3,4-thiadiazol-2-yl)methanamine

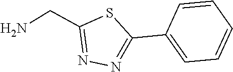 [SMILES: NCc1nnc(-c2ccccc2)s1]

### Step A: N′-(2-chloroacetyl)benzohydrazide

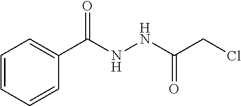 [SMILES: O=C(CCl)NNC(=O)c1ccccc1]

Chloroacetyl chloride (19.3 mL, 242 mmol) was added to a solution of benzohydrazide (30.0 g, 220 mmol) in ethyl acetate (460 mL) at 0° C. The reaction mixture was stirred and heated to 65° C. for 13 hours. The mixture was cooled to room temperature and filtered. The isolated solids were dried under reduced pressure to afford N′-(2-chloroacetyl)benzohydrazide, which was used in the next step without further purification. LC/MS (m/z): 213 (M+H)⁺

### Step B: 2-(chloromethyl)-5-phenyl-1,3,4-thiadiazole

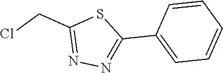 [SMILES: ClCc1nnc(-c2ccccc2)s1]

Lawesson's reagent (18.3 g, 45.1 mmol) was added to a mixture of N′-(2-chloroacetyl)benzohydrazide (16 g, 75 mmol) in THE (340 mL). The mixture was stirred and heated at 70° C. for 4 hours. The reaction mixture was quenched with saturated aqueous NaHCO₃ (300 mL) and extracted with EtOAc (3×200 mL). The organic layers were combined, washed with brine (500 mL), dried over anhydrous Na₂SO₄, filtered, and concentrated under reduced pressure. The residue was purified by silica gel chromatography (eluting ethyl acetate in petroleum ether) to afford 2-(chloromethyl)-5-phenyl-1,3,4-thiadiazole. ¹H NMR (500 MHz, chloroform-d) δ 7.99-7.94 (m, 2H), 7.54-7.47 (m, 3H), 4.98 (s, 2H). LC/MS (m/z): 211 (M+H)⁺

### Step C: (5-phenyl-1,3,4-thiadiazol-2-yl)methanamine

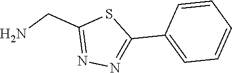 [SMILES: NCc1nnc(-c2ccccc2)s1]

A mixture of 2-(chloromethyl)-5-phenyl-1,3,4-thiadiazole (0.200 g, 0.949 mmol) in ammonia (7.0 M in MeOH, 3.0 mL, 21 mmol) was stirred and heated at 50° C. for 12 hours. The reaction mixture was cooled to room temperature and concentrated under reduced to afford (5-phenyl-1,3,4-thiadiazol-2-yl)methanamine, which was used without purification. LC/MS (m/z): 192 (M+H)⁺

### Intermediate 57

### Preparation of (5-(2H-1,2,3-triazol-2-yl)pyridin-2-yl)methanamine

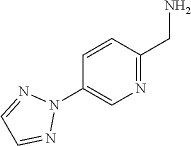 [SMILES: NCc1ccc(-n2nccn2)cn1]

### Step A: 5-(2H-1,2,3-triazol-2-yl)picolinonitrile

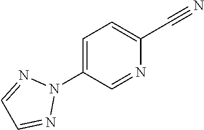 [SMILES: N#Cc1ccc(-n2nccn2)cn1]

A mixture of 2H-1,2,3-triazole (0.566 g, 8.19 mmol), Cs₂CO₃ (4.00 g, 12.3 mmol) and 5-fluoropicolinonitrile (0.50 g, 4.1 mmol) in DMF (30 mL) was stirred and heated at 80° C. for 12 hours. The mixture was cooled to room temperature and the residue was partitioned between water (50 mL) and EtOAc (30 mL). The organic layer was separated, and the aqueous was re-extracted with EtOAc (3×30 mL). The organic layers were combined, washed with brine (30 mL), dried over anhydrous Na₂SO₄, filtered, and concentrated under reduced pressure. The residue was purified by silica gel chromatography (eluting ethyl acetate in petroleum ether) to afford 5-(2H-1,2,3-triazol-2-yl)picolinonitrile. ¹H NMR (400 MHz, methanol-d₄) δ 9.46 (d, J=2.0 Hz, 1H), 8.62 (dd, J=2.5, 8.4 Hz, 1H), 8.09 (s, 2H), 8.06 (d, J=8.5 Hz, 1H). LC/MS (m/z): 172 (M+H)⁺

### Step B: tert-butyl ((5-(2H-1,2,3-triazol-2-yl)pyridin-2-yl)methyl)carbamate

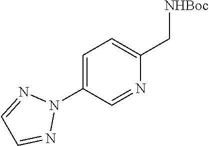 [SMILES: CC(C)(C)OC(=O)NCc1ccc(-n2nccn2)cn1]

Raney nickel (206 mg, 0.351 mmol) was added to a mixture of 5-(2H-1,2,3-triazol-2-yl)picolinonitrile (0.600 g, 3.51 mmol) and Boc-anhydride (1.6 mL, 7.0 mmol) in MeOH (20 mL) under an argon atmosphere at room temperature. The mixture was degassed and backfilled with H₂ (three times). The mixture was stirred under H₂ (50 psi) at room temperature for 16 hours. The mixture was filtered, and the filtrate was concentrated under reduced pressure. The residue was purified by silica gel chromatography (eluting ethyl acetate in petroleum ether) to afford tert-butyl ((5-(2H-1,2,3-triazol-2-yl)pyridin-2-yl)methyl)carbamate. ¹H NMR (400 MHz, DMSO-d₆) δ 9.14 (d, J=2.0 Hz, 1H), 8.39-8.38 (m, 1H), 8.19 (s, 2H), 7.56-7.44 (m, 2H), 4.30-4.29 (m, 2H), 1.41 (s, 9H). LC/MS (m/z): 276 (M+H)⁺

### Step C: (5-(2H-1,2,3-triazol-2-yl)pyridin-2-yl)methanamine

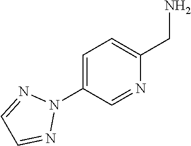 [SMILES: NCc1ccc(-n2nccn2)cn1]

A mixture of tert-butyl ((5-(2H-1,2,3-triazol-2-yl)pyridin-2-yl)methyl)carbamate (0.700 g, 2.54 mmol) in HCl (4.0 M in dioxane, 15 mL, 60 mmol) was stirred at room temperature for 2 hours. The mixture was concentrated under reduced pressure to afford (5-(2H-1,2,3-triazol-2-yl)pyridin-2-yl)methanamine, which was used without purification in the next step. ¹H NMR (400 MHz, DMSO-d₆) δ 9.26-9.25 (m, 1H), 8.55 (br s, 2H), 8.49-8.46 (m, 1H), 8.24 (s, 2H), 7.75-7.73 (m, 1H), 4.29-4.25 (m, 2H). LC/MS (m/z): 176 (M+H)⁺

### Intermediate 58

### Preparation of (5-(3-fluorophenyl)-1,3,4-thiadiazol-2-yl)methyl methanesulfonate

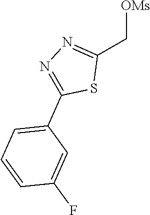 [SMILES: CS(=O)(=O)OCc1nnc(-c2cccc(F)c2)s1]

### Step A: ethyl 2-(2-(3-fluorobenzoyl)hydrazinyl)-2-oxoacetate

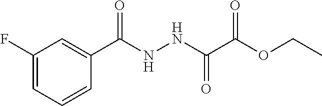 [SMILES: CCOC(=O)C(=O)NNC(=O)c1cccc(F)c1]

Ethyl 2-chloro-2-oxoacetate (5.49 mL, 49.1 mmol) was added dropwise to a mixture of 3-fluorobenzohydrazide (7.57 g, 49.1 mmol) and triethylamine (6.85 mL, 49.1 mmol) in dichloromethane (250 mL) at 0° C. The reaction mixture was stirred at 0° C. for 30 minutes after addition was complete. The reaction mixture was quenched with water (100 mL) and diluted with ethyl acetate (500 mL), methanol (50 mL), and brine (100 mL). The organic layer was separated, and the aqueous layer was extracted with additional ethyl acetate (4×100 mL). The organic layers were combined, dried over magnesium sulfate, filtered, and concentrated under reduced pressure to afford ethyl 2-(2-(3-fluorobenzoyl)hydrazineyl)-2-oxoacetate which was used without purification in the next step. ¹H NMR (500 MHz, DMSO-d₆) δ 11.02 (s, 1H), 10.71 (s, 1H), 7.74 (d, J=7.8 Hz, 1H), 7.66 (d, J=9.2 Hz, 1H), 7.63-7.58 (m, 1H), 7.52-7.48 (m, 1H), 4.31 (q, J=7.1 Hz, 2H), 1.31 (t, J=7.1 Hz, 3H). LC/MS (m/z): 255 (M+H)⁺

### Step B: ethyl 5-(3-fluorophenyl)-1,3,4-thiadiazole-2-carboxylate

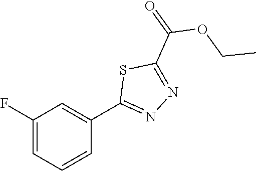 [SMILES: CCOC(=O)c1nnc(-c2cccc(F)c2)s1]

Lawesson's reagent (3.98 g, 9.83 mmol) was added to a mixture of ethyl 2-(2-(3-fluorobenzoyl)hydrazineyl)-2-oxoacetate (2.50 g, 9.83 mmol) in tetrahydrofuran (70 mL) at room temperature. The reaction mixture was stirred and heated at 70° C. for 60 minutes after addition was complete. The reaction mixture was cooled to room temperature and then concentrated under reduced pressure. The residue was purified by silica gel chromatography (eluting ethyl acetate in hexanes) to afford ethyl 5-(3-fluorophenyl)-1,3,4-thiadiazole-2-carboxylate. ¹H NMR (500 MHz, DMSO-d₆) δ 7.99-7.95 (m, 2H), 7.69-7.64 (m, 1H), 7.55-7.50 (td, J=7.8, 7.2, 1.4 Hz, 1H), 4.47 (q, J=7.1 Hz, 2H), 1.38 (t, J=7.1 Hz, 3H). LC/MS (m/z): 253 (M+H)⁺

### Step C: (5-(3-fluorophenyl)-1,3,4-thiadiazol-2-yl)methanol

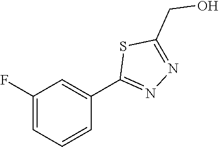 [SMILES: OCc1nnc(-c2cccc(F)c2)s1]

Sodium borohydride (1.04 g, 27.6 mmol) was added to a mixture of ethyl 5-(3-fluorophenyl)-1,3,4-thiadiazole-2-carboxylate (2.32 g, 9.20 mmol) in methanol (50 mL) at 0° C. under an argon atmosphere. The reaction mixture was stirred at 0° C. for 60 minutes after the addition was complete. The reaction mixture was quenched with acetic acid (1.58 mL, 27.6 mmol) at 0° C. The mixture was concentrated under reduced pressure (to remove methanol prior to workup). The residue was partitioned between ethyl acetate (800 mL) and brine (150 mL). The organic layer was separated, dried over magnesium sulfate, filtered, and concentrated under reduced pressure to afford (5-(3-fluorophenyl)-1,3,4-thiadiazol-2-yl)methanol, which was used without purification in the next step. ¹H NMR (499 MHz, DMSO-d₆) δ 7.87-7.81 (m, 2H), 7.65-7.58 (m, 1H), 7.46-7.41 (m, 1H), 6.34 (t, J=5.9 Hz, 1H), 4.91 (d, J=5.9 Hz, 2H). LC/MS (m/z): 211 (M+H)⁺

### Step D: (5-(3-fluorophenyl)-1,3,4-thiadiazol-2-yl)methyl methanesulfonate

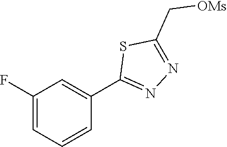 [SMILES: CS(=O)(=O)OCc1nnc(-c2cccc(F)c2)s1]

Triethylamine (2.14 mL, 15.4 mmol) was added dropwise to a mixture of (5-(3-fluorophenyl)-1,3,4-thiadiazol-2-yl)methanol (2.69 g, 12.8 mmol) and methanesulfonic anhydride (2.45 g, 14.1 mmol) in dichloromethane (100 mL) at 0° C. The reaction mixture was stirred at 0° C. for 1 hour after the addition was complete. The reaction mixture was quenched with water (20 mL), and the organic layer was separated, washed with brine (25 mL), dried over magnesium sulfate, filtered, and concentrated under reduced pressure. The residue was purified by silica gel chromatography (eluting ethyl acetate in hexanes) to afford (5-(3-fluorophenyl)-1,3,4-thiadiazol-2-yl)methyl methanesulfonate. LC/MS (m/z): 289 (M+H)⁺

### Intermediate 59

### Preparation of (3-phenylisoxazol-5-yl)methyl methanesulfonate

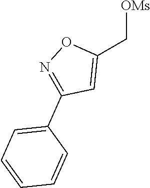 [SMILES: CS(=O)(=O)OCc1cc(-c2ccccc2)no1]

### Step A: (Z)—N-hydroxybenzimidoyl chloride

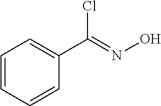

N-Chlorosuccinimide (6.17 g, 46.2 mmol) was added to a mixture of (E)-benzaldehyde oxime (5.6 g, 46 mmol) and pyridine (0.374 mL, 4.62 mmol) in THE (70 mL) at room temperature. The mixture was stirred and heated at 50° C. for 1 hour. The mixture was concentrated under reduced pressure to afford (Z)—N-hydroxybenzimidoyl chloride, which was used without purification in the next step. LC/MS (m/z): 156 (M+H)⁺

### Step B: (3-phenylisoxazol-5-yl)methanol

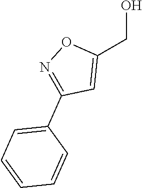 [SMILES: OCc1cc(-c2ccccc2)no1]

Prop-2-yn-1-ol (3.24 mL, 56.2 mmol) and TEA (7.73 mL, 55.5 mmol) were added to a mixture of (Z)—N-hydroxybenzimidoyl chloride (7.19 g, 46.2 mmol) in THE (50 mL) at room temperature under a nitrogen atmosphere. The mixture was stirred and heated at 50° C. for 2 hours. The mixture was cooled to room temperature and quenched by the addition of saturated aqueous sodium bicarbonate. The mixture was extracted with ethyl acetate (3×50 mL). The organic layers were combined, dried over anhydrous sodium sulfate, filtered, and concentrated under reduced pressure. The residue was purified by silica gel chromatography (eluting ethyl acetate in petroleum ether) to afford (3-phenylisoxazol-5-yl)methanol. ¹H NMR (500 MHz, chloroform-d) δ 7.70-7.42 (m, 2H), 7.27-7.26 (m, 3H), 6.36 (br d, J=17.7 Hz, 1H), 4.75-4.37 (m, 2H). LC/MS (m/z): 176 (M+H)⁺

### Step C: (3-phenylisoxazol-5-yl)methyl methanesulfonate

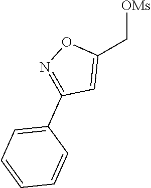 [SMILES: CS(=O)(=O)OCc1cc(-c2ccccc2)no1]

Triethylamine (1.59 mL, 11.4 mmol) was added to a mixture of (3-phenylisoxazol-5-yl)methanol (1.00 g, 5.71 mmol) and methanesulfonic anhydride (1.49 g, 8.56 mmol) in DCM (20 mL) at room temperature. The mixture was stirred at room temperature for 1 hour. The mixture was concentrated under reduced pressure, and the residue was purified by silica gel chromatography (eluting ethyl acetate in petroleum ether) to afford (3-phenylisoxazol-5-yl)methyl methanesulfonate. LC/MS (m/z): 254 (M+H)⁺

Intermediates shown in Intermediate Table 9 below, were prepared according to procedures analogous to those outlined in Intermediate 59 above using the appropriate starting materials, described in the Preparations or Intermediates above, or as obtained from commercial sources.

Intermediate Table 9

| Intermediate | Structure | Name | Mass [M + H]⁺ |
|---|---|---|---|
| Intermediate 60 | 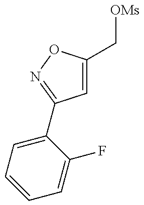 [SMILES: CS(=O)(=O)OCc1cc(-c2ccccc2F)no1] | (3-(2-fluorophenyl)isoxazol-5- yl)methyl methanesulfonate | 272 |
| Intermediate 61 | 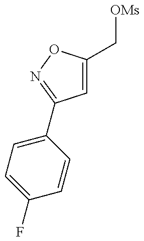 [SMILES: CS(=O)(=O)OCc1cc(-c2ccc(F)cc2)no1] | (3-(4-fluorophenyl)isoxazol-5- yl)methyl methanesulfonate | 272 |
| Intermediate 62 | 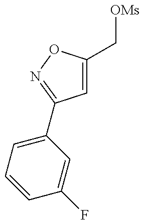 [SMILES: CS(=O)(=O)OCc1cc(-c2cccc(F)c2)no1] | (3-(3-fluorophenyl)isoxazol-5- yl)methyl methanesulfonate | 272 |
| Intermediate 63 | 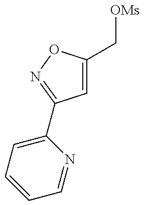 [SMILES: CS(=O)(=O)OCc1cc(-c2ccccn2)no1] | (3-(pyridin-2-yl)isoxazol-5- yl)methyl methanesulfonate | 255 |

### Intermediate 64

### Preparation of 4-(bromomethyl)-1-phenyl-1H-pyrazole

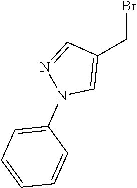 [SMILES: BrCc1cnn(-c2ccccc2)c1]

### Step A: (1-phenyl-1H-pyrazol-4-yl)methanol

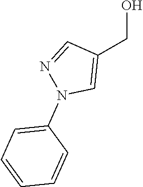 [SMILES: OCc1cnn(-c2ccccc2)c1]

Borane-THF (1.0 M in THF, 2.7 mL, 2.7 mmol) was added to a mixture of 1-phenyl-1H-pyrazole-4-carboxylic acid (0.500 g, 2.66 mmol) in THE (20 mL) at 0° C. The mixture was warmed to room temperature and stirred for 16 hours. The mixture was concentrated under reduced pressure, and the residue was purified by silica gel chromatography (eluting ethyl acetate in petroleum ether) to afford (1-phenyl-1H-pyrazol-4-yl)methanol. LC/MS (m/z): 175 (M+H)⁺

### Step B: 4-(bromomethyl)-1-phenyl-1H-pyrazole

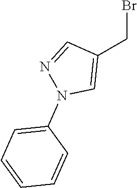 [SMILES: BrCc1cnn(-c2ccccc2)c1]

A mixture of (1-phenyl-1H-pyrazol-4-yl)methanol (0.400 g, 2.30 mmol), tetrabromomethane (1.1 g, 3.4 mmol), and triphenylphosphine (903 mg, 3.44 mmol) in DCM (20 mL) was stirred at room temperature for 16 hours. The mixture concentrated under reduced pressure, and the residue was purified by silica gel chromatography (eluting ethyl acetate in petroleum ether) to afford 4-(bromomethyl)-1-phenyl-1H-pyrazole.

### Intermediate 65

### Preparation of 5-(bromomethyl)-3-phenylisothiazole

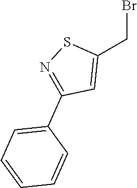 [SMILES: BrCc1cc(-c2ccccc2)ns1]

### Step A: 5-methyl-3-phenylisothiazole

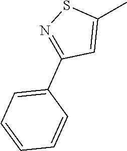

A mixture of potassium phosphate tribasic (884 mg, 4.16 mmol), Pd(dppf)Cl₂ (76 mg, 0.10 mmol), trimethylboroxine (1.52 mL, 5.21 mmol), and 5-bromo-3-phenylisothiazole (0.500 g, 2.08 mmol) in 1,4-dioxane (25 mL) was sparged with N₂. The mixture was stirred and heated to 90° C. for 24 hours. The mixture was cooled to room temperature and concentrated under reduced pressure. The residue was partitioned between water (10 mL) and EtOAc (10 mL). The organic layer was separated, and the aqueous was re-extracted with EtOAc (3×10 mL). The organic layers were combined, washed with brine (10 mL), dried over anhydrous Na₂SO₄, filtered, and concentrated under reduced pressure. The residue was purified by silica gel chromatography (eluting ethyl acetate in petroleum ether) to afford 5-methyl-3-phenylisothiazole. ¹H NMR (500 MHz, methanol-d₄) δ 7.95-7.89 (m, 2H), 7.52 (s, 1H), 7.47-7.38 (m, 3H), 2.71-2.59 (m, 3H). LC/MS (m/z): 176 (M+H)⁺

### Step B: 5-(bromomethyl)-3-phenylisothiazole

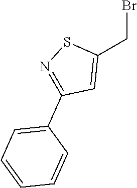 [SMILES: BrCc1cc(-c2ccccc2)ns1]

N-Bromosuccinimide (203 mg, 1.14 mmol) was added to a mixture of 5-methyl-3-phenylisothiazole (0.200 g, 1.14 mmol) and azobisisobutyronitrile (38 mg, 0.23 mmol) in chloroform (15 mL) at room temperature. The mixture was stirred at 80° C. for 2.5 hours. The mixture was cooled to room temperature and concentrated under reduced pressure. The residue was partitioned between water (10 mL) and DCM (10 mL). The organic layer was separated, and the aqueous was re-extracted with DCM (3×10 mL). The organic layers were combined, washed with brine (10 mL), dried over anhydrous Na₂SO₄, filtered, and concentrated under reduced pressure. The residue was purified by silica gel chromatography (eluting ethyl acetate in petroleum ether) to afford the crude product. The residue was purified by reverse phase (eluting acetonitrile in water, with ammonium bicarbonate modifier) to afford 5-(bromomethyl)-3-phenylisothiazole. ¹H NMR (400 MHz, chloroform-d) δ 7.94-7.89 (m, 2H), 7.59 (s, 1H), 7.49-7.39 (m, 3H), 4.74 (d, J=0.8 Hz, 2H). LC/MS (m/z): 254, 256 (M+H)⁺

### Intermediate 66

### Preparation of 1-(1-methylcyclobutyl)piperazine-2,3-dione

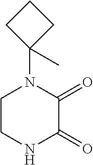

Pd(OH)₂ (0.394 g, 0.561 mmol) and palladium on carbon (1.79 g, 1.68 mmol) were added to a mixture of 1-(bicyclo[1.1.1]pentan-1-yl)-1,4-dihydropyrazine-2,3-dione (5.0 g, 28 mmol) in MeOH (200 mL) under a stream of nitrogen. The mixture was degassed and backfilled with H₂ (three times). The mixture was stirred under a H₂ atmosphere (Pressure: 50 psi) and heated at 60° C. for 24 hours. The reaction mixture was cooled to room temperature and filtered. The filtrate was concentrated under reduced pressure to afford a mixture of 1-(1-methylcyclobutyl)piperazine-2,3-dione and 1-(bicyclo[1.1.1]pentan-1-yl)piperazine-2,3-dione. The mixture was resolved by Chiral-SFC (Column Phenomenex-Cellulose-2 [250 mm×50 mm, 10 um]; eluting 45% ethanol in CO₂ with 0.1% ammonia modifier) to afford 1-(bicyclo[1.1.1]pentan-1-yl)piperazine-2,3-dione as the first eluting peak and 1-(1-methylcyclobutyl)piperazine-2,3-dione as the second eluting peak.

Peak 1: 1-(bicyclo[1.1.1]pentan-1-yl)piperazine-2,3-dione: ¹H NMR (400 MHz, DMSO-d₆) δ 8.59 (br s, 1H), 3.50-3.40 (m, 2H), 3.36-3.31 (m, 2H), 2.54-2.51 (m, 1H), 2.16-2.09 (m, 6H). LC/MS (m/z): 181 (M+H)⁺

Peak 2: 1-(1-methylcyclobutyl)piperazine-2,3-dione: ¹H NMR (400 MHz, DMSO-d₆) δ 8.52 (br s, 1H), 3.33-3.28 (m, 4H), 2.28-2.16 (m, 2H), 1.98-1.89 (m, 2H), 1.80-1.59 (m, 2H), 1.35 (s, 3H). LC/MS (m/z): 183 (M+H)⁺

### Intermediate 67

### Preparation of 3-(bromomethyl)-5-phenyl-1H-pyrazole

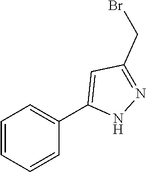 [SMILES: BrCc1cc(-c2ccccc2)[nH]n1]

### Step A: (5-phenyl-1H-pyrazol-3-yl)methanol

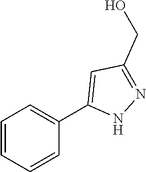 [SMILES: OCc1cc(-c2ccccc2)[nH]n1]

Borane-THF (1.0 M in THF, 2.1 mL, 2.1 mmol) was added to a mixture of 5-phenyl-1H-pyrazole-3-carboxylic acid (0.400 g, 2.13 mmol) in THE (6 mL) at 0° C. The reaction mixture was warmed to room temperature and stirred for 12 hours. The reaction was quenched with MeOH (8 mL) and heated at 60° C. for 2 hours. The mixture was concentrated under reduced pressure. The residue was diluted with water and extracted with EtOAc (3×50 mL). The organic layers were combined, dried over anhydrous Na₂SO₄, filtered, and concentrated under reduced pressure. The residue was purified by silica gel chromatography (eluting ethyl acetate in petroleum ether) to afford (5-phenyl-1H-pyrazol-3-yl)methanol.

### Step B: 3-(bromomethyl)-5-phenyl-1H-pyrazole

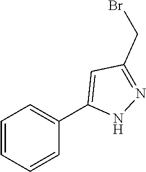 [SMILES: BrCc1cc(-c2ccccc2)[nH]n1]

A mixture of (5-phenyl-1H-pyrazol-3-yl)methanol (0.050 g, 0.29 mmol) and phosphorus tribromide (0.014 mL, 0.14 mmol) in trichloromethane (5 mL) was stirred at room temperature for 1 hour. The mixture was concentrated under reduced pressure. The residue was partitioned between water (5 mL) and EtOAc (5 mL). The organic layer was separated, and the aqueous was re-extracted with EtOAc (3×5 mL). The organic layers were combined, washed with brine (5 mL), dried over anhydrous Na₂SO₄, filtered, and concentrated under reduced pressure to afford 3-(bromomethyl)-5-phenyl-1H-pyrazole which was used in the next step without purification. LC/MS (m/z): 237, 239 (M+H)⁺

### Intermediate 68

### Preparation of (5-(pyridin-2-yl)isoxazol-3-yl)methyl methanesulfonate

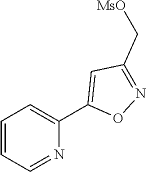 [SMILES: CS(=O)(=O)OCc1cc(-c2ccccn2)on1]

### Step A: ethyl 5-(pyridin-2-yl)isoxazole-3-carboxylate

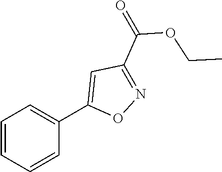 [SMILES: CCOC(=O)c1cc(-c2ccccc2)on1]

2-ethynylpyridine (10 g, 97 mmol) was added to a mixture of ethyl (E)-2-chloro-2-(hydroxyimino)acetate (15 g, 97 mmol) and TEA (14 mL, 97 mmol) in THE (150 mL) at room temperature. The mixture was stirred at room temperature for 12 hours. The reaction mixture was quenched with saturated aqueous sodium bicarbonate (50 mL) and extracted with EtOAc (100 mL). The organic layer was separated, dried over sodium sulfate, filtered, and concentrated under reduced pressure. The residue was purified by silica gel chromatography (eluting ethyl acetate in petroleum ether) to afford ethyl 5-(pyridin-2-yl)isoxazole-3-carboxylate. ¹H NMR (500 MHz, chloroform-d) δ 8.73 (d, J=4.6 Hz, 1H), 8.00-7.81 (m, 2H), 7.39 (dd, J=4.9, 7.5 Hz, 1H), 7.31 (s, 1H), 4.49 (q, J=7.1 Hz, 2H), 1.45 (t, J=7.1 Hz, 3H). LC/MS (m/z): 219 (M+H)⁺

### Step B: (5-(pyridin-2-yl)isoxazol-3-yl)methanol

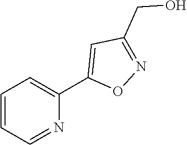 [SMILES: OCc1cc(-c2ccccn2)on1]

NaBH₄ (0.721 g, 19.1 mmol) was added to a mixture of ethyl 5-(pyridin-2-yl)isoxazole-3-carboxylate (3.2 g, 15 mmol) in MeOH (50 mL) at 0° C. The mixture was warmed to room temperature and stirred for 2 hours. The mixture was quenched with saturated aqueous ammonium chloride until the pH was ˜7. The mixture was partially concentrated under reduced pressure and then diluted with DCM (100 mL). The mixture was washed with water (10 mL). The organic layer was separated, dried over sodium sulfate, filtered, and concentrated under reduced pressure. The residue was purified by silica gel chromatography (eluting methanol in dichloromethane) to afford (5-(pyridin-2-yl)isoxazol-3-yl)methanol. ¹H NMR (400 MHz, chloroform-d) δ 8.77-8.59 (m, 1H), 7.95-7.88 (m, 1H), 7.87-7.81 (m, 1H), 7.36 (ddd, J=1.4, 4.9, 7.4 Hz, 1H), 7.00 (s, 1H), 4.85 (d, J=6.3 Hz, 2H), 2.82-2.64 (m, 1H). LC/MS (m/z): 177 (M+H)⁺

### Step C: (5-(pyridin-2-yl)isoxazol-3-yl)methyl methanesulfonate

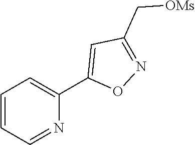 [SMILES: CS(=O)(=O)OCc1cc(-c2ccccn2)on1]

A mixture of (5-(pyridin-2-yl)isoxazol-3-yl)methanol (0.850 g, 4.82 mmol), methanesulfonic anhydride (1.26 g, 7.24 mmol), and TEA (1.35 mL, 9.65 mmol) in DCM (15 mL) was stirred at room temperature for 1 hour. The mixture was concentrated under reduced pressure, and the residue was purified by silica gel chromatography (eluting ethyl acetate in petroleum ether) to afford (5-(pyridin-2-yl)isoxazol-3-yl)methyl methanesulfonate. ¹H NMR (500 MHz, chloroform-d) δ 8.83-8.57 (m, 1H), 7.94-7.88 (m, 1H), 7.87-7.81 (m, 1H), 7.40-7.33 (m, 1H), 7.09-7.00 (m, 1H), 5.44-5.34 (m, 2H), 3.13-3.04 (m, 3H). LC/MS (m/z): 255 (M+H)⁺

Intermediates shown in Intermediate Table 10 below, were prepared according to procedures analogous to those outlined in Intermediate 68 above using the appropriate starting materials, described in the Preparations or Intermediates above, or as obtained from commercial sources.

Intermediate Table 10

| Intermediate | Structure | Name | Mass [M + H]⁺ |
|---|---|---|---|
| Intermediate 69 | 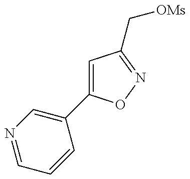 [SMILES: CS(=O)(=O)OCc1cc(-c2cccnc2)on1] | (5-(pyridin-3-yl)isoxazol-3- yl)methyl methanesulfonate | 255 |

### Intermediate 70

### Preparation of 3-(chloromethyl)-6-phenylpyridazine

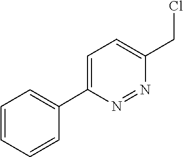 [SMILES: ClCc1ccc(-c2ccccc2)nn1]

### Step A: 3-methyl-6-phenylpyridazine

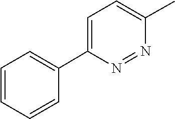

A mixture of 3-chloro-6-methylpyridazine (7.00 g, 54.4 mmol), phenylboronic acid (6.97 g, 57.2 mmol), potassium phosphate tribasic (17 g, 82 mmol), and 1,1′-bis(diphenylphosphino)ferrocenedichloro palladium(ii) dichloromethane complex (2.39 g, 3.27 mmol) in 1,4-dioxane (100 mL) was degassed with nitrogen at room temperature for 5 minutes. The mixture was stirred and heated at 80° C. for 2 hours. The reaction mixture was cooled to room temperature and diluted with water (50 mL). The mixture was extracted with EtOAc (100 mL). The organic layer was separated, washed with brine (50 mL), dried over anhydrous sodium sulfate, filtered, and concentrated under reduced pressure. The residue was diluted with 1,2-dichloroethane (100 mL). NaHSO₃ (9.8 g, 94 mmol) was added, and the mixture was stirred and heated at 65° C. for 12 hours. The mixture was cooled to room temperature, filtered, and concentrated under reduced pressure. The residue was purified by silica gel chromatography (eluting ethyl acetate in petroleum ether) to afford 3-methyl-6-phenylpyridazine. ¹H NMR (500 MHz, chloroform-d) δ 7.92 (dd, J 7.9 Hz, 1.0 Hz, 2H), 7.62 (d, J=8.7 Hz, 1H), 7.41-7.32 (m, 3H), 7.28-7.25 (m, 1H), 2.62 (s, 3H). LC/MS (m/z): 171 (M+H)⁺

### Step B: 3-(chloromethyl)-6-phenylpyridazine

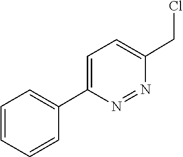 [SMILES: ClCc1ccc(-c2ccccc2)nn1]

1,3,5-trichloro-1,3,5-triazinane-2,4,6-trione (3.71 g, 16.0 mmol) was added to a mixture of 3-methyl-6-phenylpyridazine (6.8 g, 40 mmol) in trichloromethane (100 ml) at room temperature. The mixture was stirred and heated at 60° C. for 6 hours. The mixture was cooled to room temperature, filtered, and concentrated under reduced pressure. The residue was purified by silica gel chromatography (eluting ethyl acetate in petroleum ether) to afford 3-chloro-6-(chloromethyl)pyridazine. LC/MS (m/z): 205 (M+H)⁺

Intermediates shown in Intermediate Table 11 below, were prepared according to procedures analogous to those outlined in Intermediate 70 above using the appropriate starting materials, described in the Preparations or Intermediates above, or as obtained from commercial sources.

Intermediate Table 11

| Intermediate | Structure | Name | Mass [M + H]⁺ |
|---|---|---|---|
| Intermediate 71 | 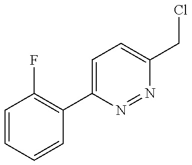 [SMILES: Fc1ccccc1-c1ccc(CCl)nn1] | 3-(chloromethyl)-6-(2- fluorophenyl)pyridazine | 223 |
| Intermediate 72 | 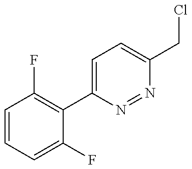 [SMILES: Fc1cccc(F)c1-c1ccc(CCl)nn1] | 3-(chloromethyl)-6-(2,6- difluorophenyl)pyridazine | 241 |
| Intermediate 73 | 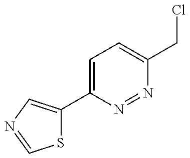 [SMILES: ClCc1ccc(-c2cncs2)nn1] | 5-(6-(chloromethyl)pyridazin-3- yl)thiazole | 212 |
| Intermediate 74 | 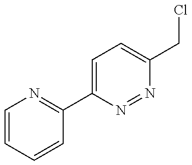 [SMILES: ClCc1ccc(-c2ccccn2)nn1] | 3-(chloromethyl)-6-(pyridin-2- yl)pyridazine | 206 |
| Intermediate 75 | 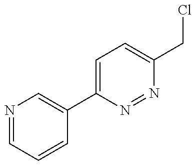 [SMILES: ClCc1ccc(-c2cccnc2)nn1] | 3-(chloromethyl)-6-(pyridin-3- yl)pyridazine | 206 |

### Intermediate 76

### Preparation of 2-(chloromethyl)-5-(2H-1,2,3-triazol-2-yl)pyri dine

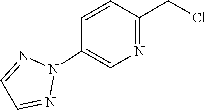 [SMILES: ClCc1ccc(-n2nccn2)cn1]

### Step A: 2-methyl-5-(2H-1,2,3-triazol-2-yl)pyri dine

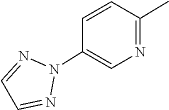

A mixture of 5-bromo-2-methylpyridine (15.9 g, 92.0 mmol), Pd₂(dba)₃ (8.46 g, 9.24 mmol), 2-di-tert-butylphosphino-3,4,5,6-tetramethyl-2′,4′,6′-triisopropyl-1,1′-biphenyl (11.1 g, 23.1 mmol), and potassium phosphate (39.2 g, 185 mmol) was degassed with argon for 5 minutes. Toluene (200 mL) and 1H-1,2,3-triazole (8.0 mL, 140 mmol) were added to the reaction mixture, and the mixture was degassed with argon for an additional 5 minutes (sub-surface sparge). The reaction mixture was stirred under an argon atmosphere and heated to 110° C. for 2.5 days. The reaction mixture was cooled to room temperature and diluted with dichloromethane (400 mL). The mixture was filtered through a fritted funnel and the filtrate was concentrated under reduced pressure. The residue was purified by silica gel chromatography (eluting methanol in dichloromethane) to afford the product as a mixture with minor impurities. The isolated material was re-purified by silica gel chromatography (eluting ethyl acetate in hexanes) to afford 2-methyl-5-(2H-1,2,3-triazol-2-yl)pyridine. ¹H NMR (500 MHz, DMSO-d₆) δ 9.10 (d, J=2.5 Hz, 1H), 8.27 (dd, J=8.4, 2.6 Hz, 1H), 8.18 (s, 2H), 7.48 (d, J=8.4 Hz, 1H), 2.55 (s, 3H). LC/MS (m/z): 161 (M+H)⁺

### Step B: 2-(chloromethyl)-5-(2H-1,2,3-triazol-2-yl)pyridine

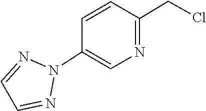 [SMILES: ClCc1ccc(-n2nccn2)cn1]

A mixture of 2-methyl-5-(2H-1,2,3-triazol-2-yl)pyridine (11.8 g, 73.4 mmol) and trichloroisocyanuric acid (17.1 g, 73.4 mmol) in carbon tetrachloride (200 mL) was stirred and heated at 80° C. for 18 hours. Additional trichloroisocyanuric acid (5.1 g, 22 mmol) was added to the reaction mixture, and the mixture was stirred for an additional 8 hours at 80° C. The reaction mixture was cooled to room temperature. The mixture was filtered through a fritted funnel, and the solids were washed with DCM (200 mL). The filtrate was concentrated under reduced pressure, and the residue was purified by silica gel chromatography (eluting ethyl acetate in dichloromethane) to afford 2-(chloromethyl)-5-(2H-1,2,3-triazol-2-yl)pyridine. ¹H NMR (500 MHz, DMSO-d₆) δ 9.23 (d, J=2.4 Hz, 1H), 8.44 (dd, J=8.5, 2.6 Hz, 1H), 8.23 (s, 2H), 7.78 (d, J=8.5 Hz, 1H), 4.88 (s, 2H). LC/MS (m/z): 195 (M+H)⁺

### Intermediate 77

### Preparation of 2-(chloromethyl)-5-phenyl-1,3,4-thiadiazole

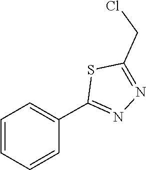 [SMILES: ClCc1nnc(-c2ccccc2)s1]

### Step A: N′-(2-chloroacetyl)benzohydrazide

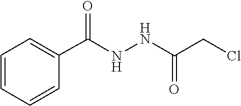 [SMILES: O=C(CCl)NNC(=O)c1ccccc1]

Chloroacetyl chloride (19.3 mL, 242 mmol) was added to a mixture of benzohydrazide (30.0 g, 220 mmol) in ethyl acetate (460 mL) at 0° C. The mixture was stirred and heated at 65° C. for 13 hours. The mixture was cooled to room temperature and filtered. The isolated solids were dried under vacuum to afford N′-(2-chloroacetyl)benzohydrazide, which was used in the next step without further purification. LC/MS (m/z): 213 (M+H)⁺

### Step B: 2-(chloromethyl)-5-phenyl-1,3,4-thiadiazole

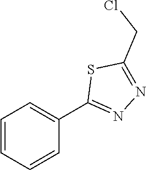 [SMILES: ClCc1nnc(-c2ccccc2)s1]

Lawesson's reagent (18.3 g, 45.1 mmol) was added to a mixture of N′-(2-chloroacetyl)benzohydrazide (16 g, 75 mmol) in THE (340 mL). The mixture was stirred and heated at 70° C. for 4 hours. The mixture was cooled to room temperature, diluted with saturated aqueous NaHCO₃ (300 mL), and extracted with EtOAc (3×200 mL). The organic layers were combined, washed with brine (500 mL), dried over anhydrous Na₂SO₄, filtered, and concentrated under reduced pressure. The residue was purified by silica gel chromatography (eluting ethyl acetate in petroleum ether) to afford 2-(chloromethyl)-5-phenyl-1,3,4-thiadiazole. ¹H NMR (500 MHz, chloroform-d) δ 7.99-7.94 (m, 2H), 7.54-7.47 (m, 3H), 4.98 (s, 2H). LC/MS (m/z): 211 (M+H)⁺

Intermediates shown in Intermediate Table 12 below, were prepared according to procedures analogous to those outlined in Intermediate 77 above using the appropriate starting materials, described in the Preparations or Intermediates above, or as obtained from commercial sources.

Intermediate Table 12

| Intermediate | Structure | Name | Mass [M + H]⁺ |
|---|---|---|---|
| Intermediate 78 | 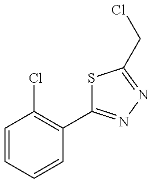 [SMILES: ClCc1nnc(-c2ccccc2Cl)s1] | 2-(chloromethyl)-5-(2- chlorophenyl)-1,3,4-thiadiazole | 245 |
| Intermediate 79 | 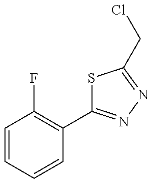 [SMILES: Fc1ccccc1-c1nnc(CCl)s1] | 2-(chloromethyl)-5-(2- fluorophenyl)-1,3,4-thiadiazole | 229 |

### Intermediate 80

### Preparation of 2-(chloromethyl)-5-(3-fluorophenyl)-1,3,4-thiadiazole

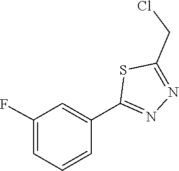 [SMILES: Fc1cccc(-c2nnc(CCl)s2)c1]

### Step A: N′-(2-chloroacetyl)-3-fluorobenzohydrazide

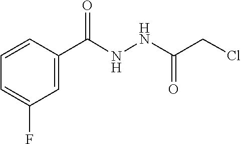 [SMILES: O=C(CCl)NNC(=O)c1cccc(F)c1]

2-Chloroacetyl chloride (2.58 mL, 32.4 mmol) was added dropwise to a mixture of 3-fluorobenzohydrazide (5.00 g, 32.4 mmol) in EtOAc (50 mL) at 0° C. The mixture was stirred at room temperature for 7 hours. The mixture was cooled to room temperature and concentrated under reduced pressure to afford N′-(2-chloroacetyl)-3-fluorobenzohydrazide, which was used in the next step with purification. LC/MS (m/z): 231 (M+H)⁺

### Step B: 2-(chloromethyl)-5-(3-fluorophenyl)-1,3,4-thiadiazole

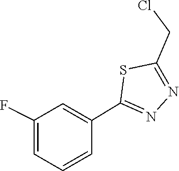 [SMILES: Fc1cccc(-c2nnc(CCl)s2)c1]

Lawesson's reagent (8.52 g, 21.07 mmol) was added portion wise to a mixture of N′-(2-chloroacetyl)-3-fluorobenzohydrazide (8.10 g, 35.1 mmol) in THE (90 mL) at 0° C. The reaction mixture was stirred and heated at 80° C. for 12 hours. The reaction mixture was cooled to room temperature, diluted with saturated aqueous NaHCO₃ (50 mL), and extracted with EtOAc (3×50 mL). The organic layers were combined, washed with brine (50 mL), dried over anhydrous Na₂SO₄, filtered, and concentrated under reduced pressure. The residue was purified by silica gel chromatography (eluting ethyl acetate in petroleum ether) to afford 2-(chloromethyl)-5-(3-fluorophenyl)-1,3,4-thiadiazole. ¹H NMR (500 MHz, methanol-d₄) δ 7.83-7.75 (m, 2H), 7.61-7.54 (m, 1H), 7.33 (dt, J=8.5 Hz, 2.4 Hz, 1H), 5.13 (s, 2H). LC/MS (m/z): 229 (M+H)⁺

### Intermediate 81

### Preparation of (3-phenylbicyclo[1.1.1]pentan-1-yl)methyl 4-methylbenzenesulfonate

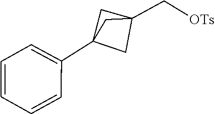 [SMILES: Cc1ccc(S(=O)(=O)OCC23CC(c4ccccc4)(C2)C3)cc1]

### Step A: (3-phenylbicyclo[1.1.1]pentan-1-yl)methanol

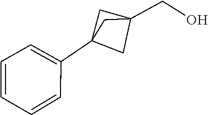 [SMILES: OCC12CC(c3ccccc3)(C1)C2]

Borane-THF (1.0 M in THF, 1.6 mL, 1.6 mmol) was added dropwise to a mixture of 3-phenylbicyclo[1.1.1]pentane-1-carboxylic acid (0.15 g, 0.80 mmol) in THE (4 mL) at 0° C. The mixture was stirred for 1 hour at 0° C. and then warmed to room temperature and stirred for 1 hour. The mixture was quenched by the dropwise addition of MeOH (5 mL), and the mixture was concentrated under reduced pressure. Saturated aqueous K₂CO₃ (20 mL) was added to the residue, and the mixture was extracted with DCM (2×10 mL). The organic layers were combined, dried over anhydrous Na₂SO₄, filtered, and concentrated under reduced pressure to afford (3-phenylbicyclo[1.1.1]pentan-1-yl)methanol, which was used in the next step without purification. LC/MS (m/z): 157 (M+H-H₂O)⁺

### Step B: (3-phenylbicyclo[1.1.1]pentan-1-yl)methyl 4-methylbenzenesulfonate

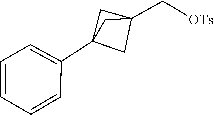 [SMILES: Cc1ccc(S(=O)(=O)OCC23CC(c4ccccc4)(C2)C3)cc1]

4-Toluenesulfonyl chloride (171 mg, 0.895 mmol) was added to a mixture of (3-phenylbicyclo[1.1.1]pentan-1-yl)methanol (0.120 g, 0.689 mmol) and TEA (0.192 ml, 1.377 mmol) in DCM (4 mL) at room temperature. The mixture was stirred for 12 hours at room temperature. The mixture was concentrated under reduced pressure, and the residue was purified by silica gel chromatography (eluting ethyl acetate in petroleum ether) to afford (3-phenylbicyclo[1.1.1]pentan-1-yl)methyl 4-methylbenzenesulfonate. ¹H NMR (500 MHz, chloroform-d) δ 7.81 (d, J=8.2 Hz, 2H), 7.35 (d, J=8.1 Hz, 2H), 7.31-7.27 (m, 2H), 7.23-7.19 (m, 1H), 7.19-7.15 (m, 2H), 4.11 (s, 2H), 2.46 (s, 3H), 1.97 (s, 6H). LC/MS (m/z): 346 (M+H₂O)⁺

### Intermediate 82

### Preparation of 1-((6-chloropyridazin-3-yl)methyl)-4-cyclopentylpiperazine-2,3-dione

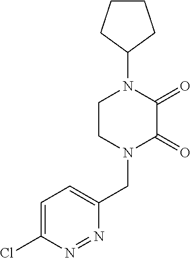 [SMILES: O=C1C(=O)N(C2CCCC2)CCN1Cc1ccc(Cl)nn1]

Sodium hydride (27 mg, 0.68 mmol, 60% dispersion in mineral oil) was added to a mixture of 1-cyclopentylpiperazine-2,3-dione (0.100 g, 0.549 mmol) in DMF (5 mL) at 0° C. The mixture was stirred for 5 minutes, and then 3-chloro-6-(chloromethyl)pyridazine (89 mg, 0.55 mmol) was added at room temperature. The reaction mixture was stirred at room temperature for 12 hours. The reaction mixture was quenched with water (20 mL) and extracted with EtOAc (3×25 mL). The organic layers were combined, washed with brine (10 mL), dried over anhydrous sodium sulfate, filtered, and concentrated under reduced pressure. The residue was purified by reverse phase HPLC (eluting acetonitrile in water, with TFA modifier) to afford 1-((6-chloropyridazin-3-yl)methyl)-4-cyclopentylpiperazine-2,3-dione. ¹H NMR (400 MHz, methanol-d₄) δ 7.83-7.71 (i, 2H), 4.94 (s, 2H), 4.82-4.78 (m, 1H), 3.76-3.70 (m, 2H), 3.63-3.56 (m, 2H), 1.95-1.86 (m, 2H), 1.82-1.73 (m, 2H), 1.68-1.60 (in, 4H). LC/MS (m/z): 309 (M+H)⁺

Examples shown in Intermediate Table 13 below, were prepared according to procedures analogous to those outlined in Intermediate 82 above using the appropriate starting materials, described in the Preparations or Intermediates above, or as obtained from commercial sources.

Intermediate Table 13

| Intermediate | Structure | Name | Mass [M + H]⁺ |
|---|---|---|---|
| Intermediate 83 | 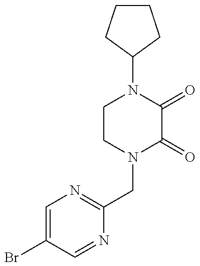 [SMILES: O=C1C(=O)N(C2CCCC2)CCN1Cc1ncc(Br)cn1] | 1-((5-bromopyrimidin-2-yl)methyl)-4- cyclopentylpiperazine-2,3-dione | 353, 355 |
| Intermediate 84 | 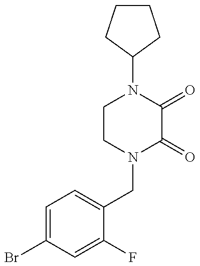 [SMILES: O=C1C(=O)N(C2CCCC2)CCN1Cc1ccc(Br)cc1F] | 1-(4-bromo-2-fluorobenzyl)-4- cyclopentylpiperazine-2,3-dione | 369, 371 |
| Intermediate 85 | 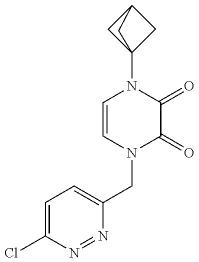 [SMILES: O=c1c(=O)n(C23CC(C2)C3)ccn1Cc1ccc(Cl)nn1] | 1-(bicyclo[1.1.1]pentan-1-yl)-4-((6- chloropyridazin-3-yl)methyl)-1,4- dihydropyrazine-2,3-dione | 305 |
| Intermediate 86 | 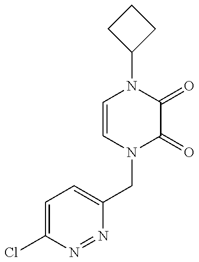 [SMILES: O=c1c(=O)n(C2CCC2)ccn1Cc1ccc(Cl)nn1] | 1-((6-chloropyridazin-3-yl)methyl)-4- cyclobutyl-1,4-dihydropyrazine-2,3- dione | 293 |
| Intermediate 87 | 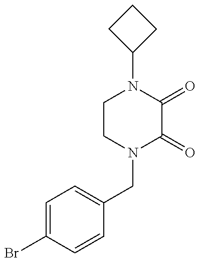 [SMILES: O=C1C(=O)N(C2CCC2)CCN1Cc1ccc(Br)cc1] | 1-(4-bromobenzyl)-4- cyclobutylpiperazine-2,3-dione | 337, 339 |
| Intermediate 88 | 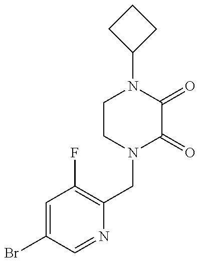 [SMILES: O=C1C(=O)N(C2CCC2)CCN1Cc1ncc(Br)cc1F] | 1-((5-bromo-3-fluoropyridin-2- yl)methyl)-4-cyclobutylpiperazine- 2,3-dione | 356, 358 |
| Intermediate 89 | 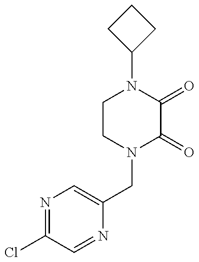 [SMILES: O=C1C(=O)N(C2CCC2)CCN1Cc1cnc(Cl)cn1] | 1-((5-chloropyrazin-2-yl)methyl)-4- cyclobutylpiperazine-2,3-dione | 295 |
| Intermediate 90 | 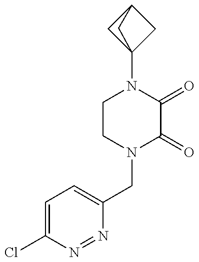 [SMILES: O=C1C(=O)N(C23CC(C2)C3)CCN1Cc1ccc(Cl)nn1] | 1-(bicyclo[1.1.1]pentan-1-yl)-4-((6- chloropyridazin-3- yl)methyl)piperazine-2,3-dione | 307 |
| Intermediate 91 | 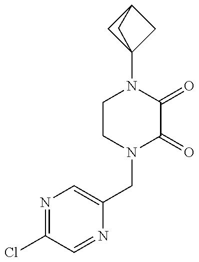 [SMILES: O=C1C(=O)N(C23CC(C2)C3)CCN1Cc1cnc(Cl)cn1] | 1-(bicyclo[1.1.1]pentan-1-yl)-4-((5- chloropyrazin-2-yl)methyl)piperazine- 2,3-dione | 307 |
| Intermediate 92 | 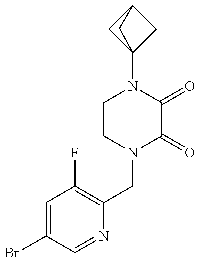 [SMILES: O=C1C(=O)N(C23CC(C2)C3)CCN1Cc1ncc(Br)cc1F] | 1-(bicyclo[1.1.1]pentan-1-yl)-4-((5- bromo-3-fluoropyridin-2- yl)methyl)piperazine-2,3-dione | 368, 370 |
| Intermediate 93 | 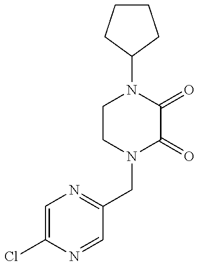 [SMILES: O=C1C(=O)N(C2CCCC2)CCN1Cc1cnc(Cl)cn1] | 1-((5-chloropyrazin-2-yl)methyl)-4- cyclopentylpiperazine-2,3-dione | 309 |
| Intermediate 94 | 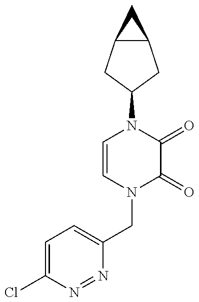 [SMILES: O=c1c(=O)n([C@H]2C[C@H]3C[C@H]3C2)ccn1Cc1ccc(Cl)nn1] | 1-((cis)-bicyclo[3.1.0]hexan-3-yl)-4- ((6-chloropyridazin-3-yl)methyl)-1,4- dihydropyrazine-2,3-dione | 319 |
| Intermediate 95 | 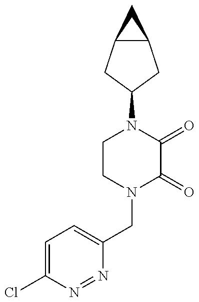 [SMILES: O=C1C(=O)N([C@H]2C[C@H]3C[C@H]3C2)CCN1Cc1ccc(Cl)nn1] | 1-((cis)-bicyclo[3.1.0]hexan-3-yl)-4- ((6-chloropyridazin-3- yl)methyl)piperazine-2,3-dione | 321 |
| Intermediate 96 | 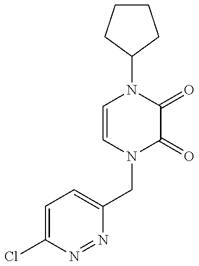 [SMILES: O=c1c(=O)n(C2CCCC2)ccn1Cc1ccc(Cl)nn1] | 1-((6-chloropyridazin-3-yl)methyl)-4- cyclopentyl-1,4-dihydropyrazine-2,3- dione | 307 |

### Intermediate 97

### Preparation of 1-((6-chloropyridazin-3-yl)methyl)-4-cyclobutylpiperazine-2,3-di one

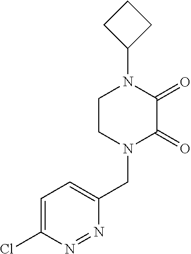 [SMILES: O=C1C(=O)N(C2CCC2)CCN1Cc1ccc(Cl)nn1]

3-Chloro-6-(chloromethyl)pyridazine (58.6 g, 359 mmol) was added portion wise to a mixture of 1-cyclobutylpiperazine-2,3-dione (55.0 g, 327 mmol) and Cs₂CO₃ (127 g, 392 mmol) in DMF (600 mL) at room temperature. The mixture was heated to 80° C. and stirred for 16 hours. The reaction mixture was cooled to room temperature and diluted with dichloromethane (400 mL) and then filtered through Celite. The filtrate was washed with water, and the aqueous layer was extracted with additional dichloromethane (2×100 mL). The organic layers were combined, dried over anhydrous sodium sulfate, filtered, and concentrated under reduced pressure. The residue was purified by silica gel chromatography (eluting methanol in dichloromethane), and the isolated product was then triturated with ethyl acetate (100 mL). The suspension was stirred at room temperature for 20 minutes and then filtered. The collected solids were dried under vacuum to afford 1-((6-chloropyridazin-3-yl)methyl)-4-cyclobutylpiperazine-2,3-dione. ¹H NMR (400 MHz, methanol-d₄) δ 7.81 (d, J=8.8 Hz, 1H), 7.76 (d, J=8.8 Hz, 1H), 4.93 (s, 2H), 4.72-4.64 (m, 1H), 3.76-3.74 (m, 2H), 3.71-3.69 (m, 2H), 2.31-2.17 (m, 4H), 1.78-1.76 (m, 2H). LC/MS (m/z): 295 (M+H)⁺

### Intermediate 98

### Preparation of 1-((6-chloropyridin-3-yl)methyl)-4-cyclopentylpiperazine-2,3-dione

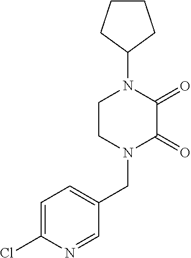 [SMILES: O=C1C(=O)N(C2CCCC2)CCN1Cc1ccc(Cl)nc1]

A mixture of 1-cyclopentylpiperazine-2,3-dione (0.73 g, 4.0 mmol), 5-(bromomethyl)-2-chloropyridine (0.90 g, 4.4 mmol) and potassium carbonate (1.82 g, 13.1 mmol) in DMF (10 mL) was stirred and heated to 30° C. for 18 hours. The mixture was cooled, diluted with water, and extracted with isopropyl alcohol:chloroform (1:3). The organic layers were combined, washed with brine, dried over magnesium sulfate, filtered, and concentrated under reduced pressure. The residue was purified by silica gel chromatography (eluting EtOAc:EtOH (3:1) in hexanes) to afford 1-((6-chloropyridin-3-yl)methyl)-4-cyclopentylpiperazine-2,3-dione. LC/MS (m/z): 308 (M+H)⁺

Examples shown in Intermediate Table 14 below, were or may be prepared according to procedures analogous to those outlined in Intermediate 98 above using the appropriate starting materials, described in the Preparations or Intermediates above, or as obtained from commercial sources.

Intermediate Table 14

| Intermediate | Structure | Name | Mass [M + H]⁺ |
|---|---|---|---|
| Intermediate 99 | 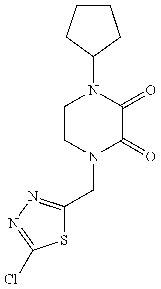 [SMILES: O=C1C(=O)N(C2CCCC2)CCN1Cc1nnc(Cl)s1] | 1-((5-chloro-1,3,4-thiadiazol-2- yl)methyl)-4-cyclopentylpiperazine- 2,3-dione | 315 |
| Intermediate 100 | 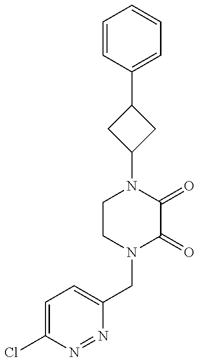 [SMILES: O=C1C(=O)N(C2CC(c3ccccc3)C2)CCN1Cc1ccc(Cl)nn1] | 1-((6-chloropyridazin-3-yl)methyl)-4- (3-phenylcyclobutyl)piperazine-2,3- dione | 371 |
| Intermediate 101 | 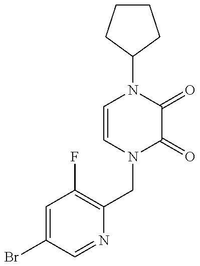 [SMILES: O=c1c(=O)n(C2CCCC2)ccn1Cc1ncc(Br)cc1F] | 1-((5-bromo-3-fluoropyridin-2- yl)methyl)-4-cyclopentylpiperazine- 2,3-dione | 370, 372 |
| Intermediate 102 | 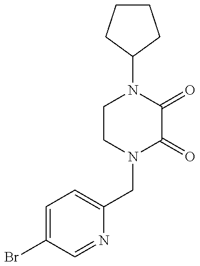 [SMILES: O=C1C(=O)N(C2CCCC2)CCN1Cc1ccc(Br)cn1] | 1-((5-bromopyridin-2-yl)methyl)-4- cyclopentylpiperazine-2,3-dione | 352, 354 |
| Intermediate 103 | 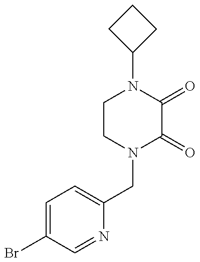 [SMILES: O=C1C(=O)N(C2CCC2)CCN1Cc1ccc(Br)cn1] | 1-((5-bromopyridin-2-yl)methyl)-4- cyclobutylpiperazine-2,3-dione | 338, 340 |
| Intermediate 104 | 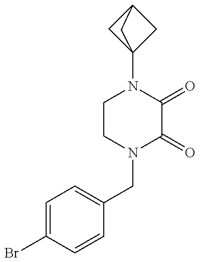 [SMILES: O=C1C(=O)N(C23CC(C2)C3)CCN1Cc1ccc(Br)cc1] | 1-(bicyclo[1.1.1]pentan-1-yl)-4-(4- bromobenzyl)piperazine-2,3-dione | 349, 351 |
| Intermediate 105 | 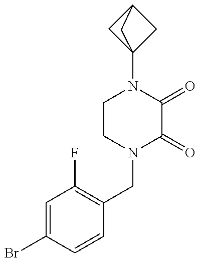 [SMILES: O=C1C(=O)N(C23CC(C2)C3)CCN1Cc1ccc(Br)cc1F] | 1-(bicyclo[1.1.1]pentan-1-yl)-4-(4- bromo-2-fluorobenzyl)piperazine- 2,3-dione | 367, 369 |
| Intermediate 106 | 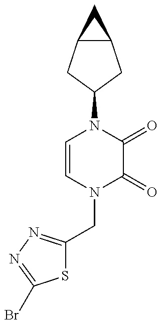 [SMILES: O=c1c(=O)n([C@H]2C[C@H]3C[C@H]3C2)ccn1Cc1nnc(Br)s1] | 1-((cis)-bicyclo[3.1.0]hexan-3-yl)-4- ((5-bromo-1,3,4-thiadiazol-2- yl)methyl)-1,4-dihydropyrazine-2,3- dione | 369, 371 |
| Intermediate 107 | 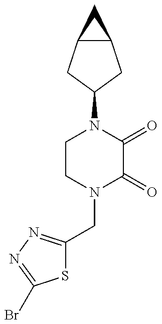 [SMILES: O=C1C(=O)N([C@H]2C[C@H]3C[C@H]3C2)CCN1Cc1nnc(Br)s1] | 1-((cis)-bicyclo[3.1.0]hexan-3-yl)-4- ((5-bromo-1,3,4-thiadiazol-2- yl)methyl)piperazine-2,3-dione | 371, 373 |
| Intermediate 108 | 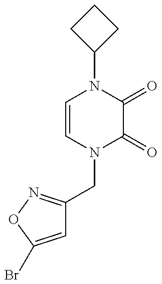 [SMILES: O=c1c(=O)n(C2CCC2)ccn1Cc1cc(Br)on1] | 1-((5-bromoisoxazol-3-yl)methyl)-4- cyclobutyl-1,4-dihydropyrazine-2,3- dione | 326, 328 |
| Intermediate 109 | 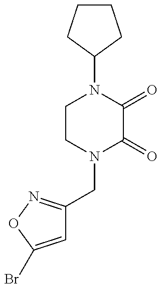 [SMILES: O=C1C(=O)N(C2CCCC2)CCN1Cc1cc(Br)on1] | 1-((5-bromoisoxazol-3-yl)methyl)-4- cyclopentylpiperazine-2,3-dione | 342, 344 |
| Intermediate 110 | 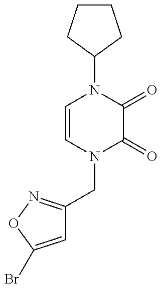 [SMILES: O=c1c(=O)n(C2CCCC2)ccn1Cc1cc(Br)on1] | 1-((5-bromoisoxazol-3-yl)methyl)-4- cyclopentyl-1,4-dihydropyrazine-2,3- dione | 340, 342 |
| Intermediate 111 | 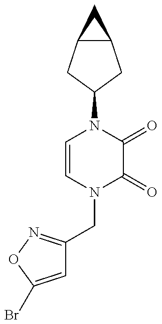 [SMILES: O=c1c(=O)n([C@H]2C[C@H]3C[C@H]3C2)ccn1Cc1cc(Br)on1] | 1-((cis)-bicyclo[3.1.0]hexan-3-yl)-4- ((5-bromoisoxazol-3-yl)methyl)-1,4- dihydropyrazine-2,3-dione | 352, 354 |
| Intermediate 112 | 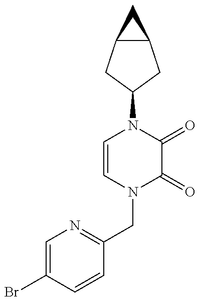 [SMILES: O=c1c(=O)n([C@H]2C[C@H]3C[C@H]3C2)ccn1Cc1ccc(Br)cn1] | 1-((cis)-bicyclo[3.1.0]hexan-3-yl)-4- ((5-bromopyridin-2-yl)methyl)-1,4- dihydropyrazine-2,3-dione | 362, 364 |
| Intermediate 113 | 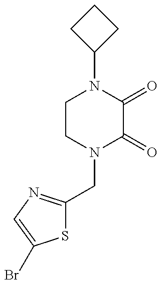 [SMILES: O=C1C(=O)N(C2CCC2)CCN1Cc1ncc(Br)s1] | 1-((5-bromothiazol-2-yl)methyl)-4- cyclobutylpiperazine-2,3-dione | 344, 346 |
| Intermediate 114 | 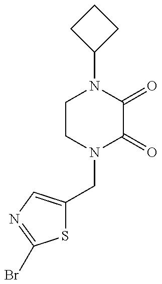 [SMILES: O=C1C(=O)N(C2CCC2)CCN1Cc1cnc(Br)s1] | 1-((2-bromothiazol-5-yl)methyl)-4- cyclobutylpiperazine-2,3-dione | 344, 346 |
| Intermediate 115 | 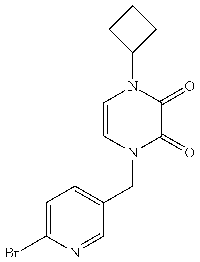 [SMILES: O=c1c(=O)n(C2CCC2)ccn1Cc1ccc(Br)nc1] | 1-((6-bromopyridin-3-yl)methyl)-4- cyclobutyl-1,4-dihydropyrazine-2,3- dione | 336, 338 |
| Intermediate 116 | 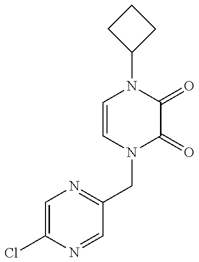 [SMILES: O=c1c(=O)n(C2CCC2)ccn1Cc1cnc(Cl)cn1] | 1-((5-chloropyrazin-2-yl)methyl)-4- cyclobutyl-1,4-dihydropyrazine-2,3- dione | 293 |
| Intermediate 117 | 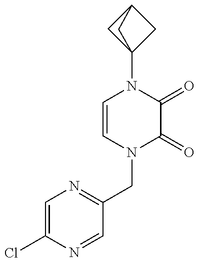 [SMILES: O=c1c(=O)n(C23CC(C2)C3)ccn1Cc1cnc(Cl)cn1] | 1-(bicyclo[1.1.1]pentan-1-yl)-4-((5- chloropyrazin-2-yl)methyl)-1,4- dihydropyrazine-2,3-dione | 305 |
| Intermediate 118 | 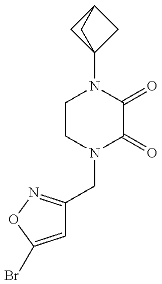 [SMILES: O=C1C(=O)N(C23CC(C2)C3)CCN1Cc1cc(Br)on1] | 1-(bicyclo[1.1.1]pentan-1-yl)-4-((5- bromoisoxazol-3- yl)methyl)piperazine-2,3-dione | 340, 342 |
| Intermediate 119 | 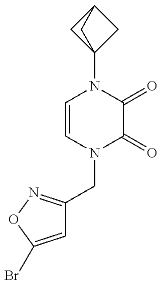 [SMILES: O=c1c(=O)n(C23CC(C2)C3)ccn1Cc1cc(Br)on1] | 1-(bicyclo[1.1.1]pentan-1-yl)-4-((5- bromoisoxazol-3-yl)methyl)-1,4- dihydropyrazine-2,3-dione | 338, 340 |
| Intermediate 120 | 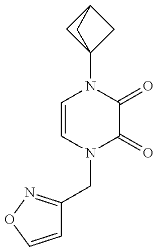 [SMILES: O=c1c(=O)n(C23CC(C2)C3)ccn1Cc1ccon1] | 1-(bicyclo[1.1.1]pentan-1-yl)-4- (isoxazol-3-ylmethyl)-1,4- dihydropyrazine-2,3-dione | 260 |
| Intermediate 121 | 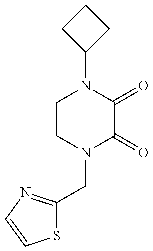 [SMILES: O=C1C(=O)N(C2CCC2)CCN1Cc1nccs1] | 1-cyclobutyl-4-(thiazol-2- ylmethyl)piperazine-2,3-dione | 266 |
| Intermediate 122 | 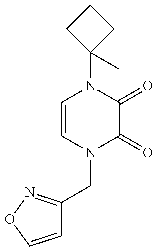 [SMILES: CC1(n2ccn(Cc3ccon3)c(=O)c2=O)CCC1] | 1-(isoxazol-3-ylmethyl)-4-(1- methylcyclobutyl)-1,4- dihydropyrazine-2,3-dione | 262 |
| Intermediate 123 | 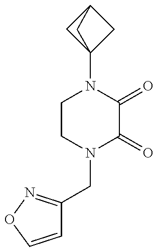 [SMILES: O=C1C(=O)N(C23CC(C2)C3)CCN1Cc1ccon1] | 1-(bicyclo[1.1.1]pentan-1-yl)-4- (isoxazol-3-ylmethyl)piperazine-2,3- dione | 262 |
| Intermediate 124 | 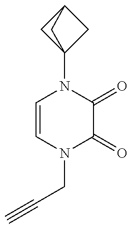 [SMILES: C#CCn1ccn(C23CC(C2)C3)c(=O)c1=O] | 1-(bicyclo[1.1.1]pentan-1-yl)-4- (prop-2-yn-1-yl)-1,4- dihydropyrazine-2,3-dione | 217 |
| Intermediate 125 | 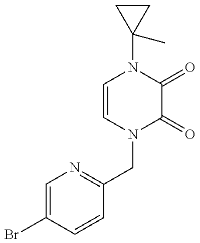 [SMILES: CC1(n2ccn(Cc3ccc(Br)cn3)c(=O)c2=O)CC1] | 1-((5-bromopyridin-2-yl)methyl)-4- (1-methylcyclopropyl)-1,4- dihydropyrazine-2,3-dione | 336, 338 |
| Intermediate 126 | 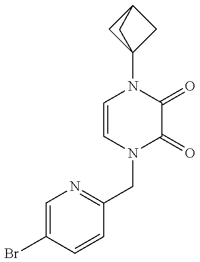 [SMILES: O=c1c(=O)n(C23CC(C2)C3)ccn1Cc1ccc(Br)cn1] | 1-(bicyclo[1.1.1]pentan-1-yl)-4-((5- bromopyridin-2-yl)methyl)-1,4- dihydropyrazine-2,3-dione | 348, 350 |
| Intermediate 127 | 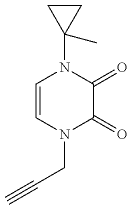 | 1-(1-methylcyclopropyl)-4-(prop-2- yn-1-yl)-1,4-dihydropyrazine-2,3- dione | 205 |
| Intermediate 128 | 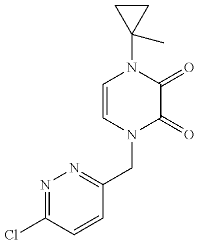 [SMILES: CC1(n2ccn(Cc3ccc(Cl)nn3)c(=O)c2=O)CC1] | 1-((6-chloropyridazin-3-yl)methyl)-4- (1-methylcyclopropyl)-1,4- dihydropyrazine-2,3-dione | 293 |
| Intermediate 129 | 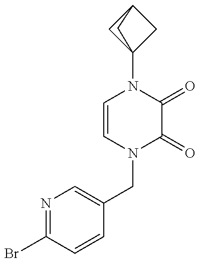 [SMILES: O=c1c(=O)n(C23CC(C2)C3)ccn1Cc1ccc(Br)nc1] | 1-(bicyclo[1.1.1]pentan-1-yl)-4-((6- bromopyridin-3-yl)methyl)-1,4- dihydropyrazine-2,3-dione | 348, 350 |
| Intermediate 130 | 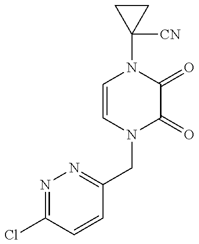 [SMILES: N#CC1(n2ccn(Cc3ccc(Cl)nn3)c(=O)c2=O)CC1] | 1-(4-((6-chloropyridazin-3- yl)methyl)-2,3-dioxo-3,4- dihydropyrazin-1(2H)- yl)cyclopropane-1-carbonitrile | 304 |
| Intermediate 131 | 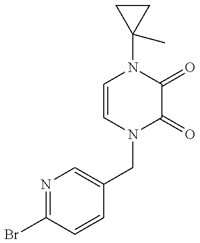 [SMILES: CC1(n2ccn(Cc3ccc(Br)nc3)c(=O)c2=O)CC1] | 1-((6-bromopyridin-3-yl)methyl)-4- (1-methylcyclopropyl)-1,4- dihydropyrazine-2,3-dione | 336, 338 |
| Intermediate 132 | 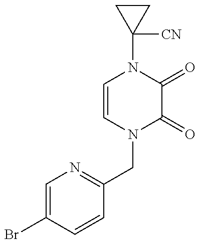 [SMILES: N#CC1(n2ccn(Cc3ccc(Br)cn3)c(=O)c2=O)CC1] | 1-(4-((5-bromopyridin-2-yl)methyl)- 2,3-dioxo-3,4-dihydropyrazin-1(2H)- yl)cyclopropane-1-carbonitrile | 347, 349 |
| Intermediate 133 | 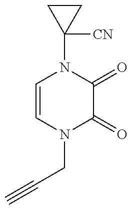 [SMILES: C#CCn1ccn(C2(C#N)CC2)c(=O)c1=O] | 1-(2,3-dioxo-4-(prop-2-yn-1-yl)-3,4- dihydropyrazin-1(2H)- yl)cyclopropane-1-carbonitrile | 216 |
| Intermediate 134 | 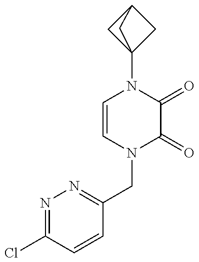 [SMILES: O=c1c(=O)n(C23CC(C2)C3)ccn1Cc1ccc(Cl)nn1] | 1-(bicyclo[1.1.1]pentan-1-yl)-4-((6- chloropyridazin-3-yl)methyl)-1,4- dihydropyrazine-2,3-dione | 305 |
| Intermediate 135 | 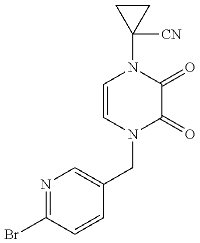 [SMILES: N#CC1(n2ccn(Cc3ccc(Br)nc3)c(=O)c2=O)CC1] | 1-(4-((6-bromopyridin-3-yl)methyl)- 2,3-dioxo-3,4-dihydropyrazin-1(2H)- yl)cyclopropane-1-carbonitrile | 347, 349 |

### Intermediate 136

### Preparation of ethyl 2-oxo-2-((2-oxoethyl)((5-phenylpyrimidin-2-yl)methyl)amino)acetate

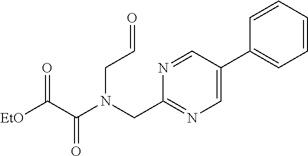 [SMILES: CCOC(=O)C(=O)N(CC=O)Cc1ncc(-c2ccccc2)cn1]

### Step A: 5-phenylpyrimidine-2-carbonitrile

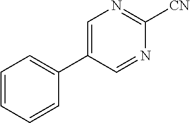 [SMILES: N#Cc1ncc(-c2ccccc2)cn1]

A mixture of 5-bromopyrimidine-2-carbonitrile (150 g, 0.81 mol), phenyl boronic acid (198 g, 1.63 mol), and potassium phosphate tribasic (346 g, 1.63 mol) in 1,4-dioxane (1.3 L) and water (0.2 L) was sparged with nitrogen for 5 minutes at room temperature. PdCl₂(dppf) (66 g, 0.080 mol) was added to the reaction mixture, and the mixture was stirred and heated at 80° C. for 30 minutes. The reaction mixture was cooled to room temperature and quenched with water (1.0 L). The mixture was extracted with ethyl acetate (3×1.0 L). The organic layers were combined, washed with brine (500 mL), dried over anhydrous sodium sulfate, filtered, and concentrated under reduced pressure. The residue was triturated with methanol, filtered, and the collected solids were dried under vacuum to afford 5-phenylpyrimidine-2-carbonitrile. ¹H NMR (400 MHz, chloroform-d) δ 9.02 (s, 2H), 7.63-7.54 (m, 5H).

### Step B: (5-phenylpyrimidin-2-yl)methanamine

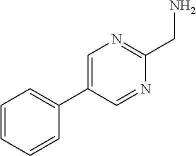 [SMILES: NCc1ncc(-c2ccccc2)cn1]

A mixture of 5-phenylpyrimidine-2-carbonitrile (16 g. 0.086 mol) and palladium on carbon (10%, 1.50 g) was sparged with nitrogen. Methanol (300 mL) was added, and the mixture was purged with hydrogen gas. The reaction mixture was stirred at room temperature under a hydrogen atmosphere for 4 hours. The reaction mixture was filtered through Celite (washing with methanol). The filtrate was concentrated under reduced pressure to afford (5-phenylpyrimidin-2-yl)methanamine, which was used without purification in the next step. ¹H NMR (400 MHz, chloroform-d) δ 9.90 (s, 2H), 7.58-7.45 (m, 5H), 4.17 (s, 2H).

### Step C: ethyl 2-oxo-2-(((5-phenylpyrimidin-2-yl)methyl)amino)acetate

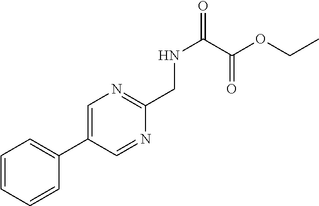 [SMILES: CCOC(=O)C(=O)NCc1ncc(-c2ccccc2)cn1]

Ethyl 2-chloro-2-oxoacetate (66 g, 0.48 mol) was added dropwise to a mixture of (5-phenylpyrimidin-2-yl)methanamine (90 g, 0.48 mol) in DCM (900 mL) at 0° C. Hunig's base (82 g, 0.63 mol) was then added to the reaction mixture at 0° C. The reaction mixture was warmed to room temperature and stirred for 1 hour. The reaction mixture was quenched with saturated aqueous sodium bicarbonate and extracted with DCM (3×300 mL). The organic layers were combined, dried over anhydrous sodium sulfate, filtered, and concentrated under reduced pressure. The residue was triturated with ethyl acetate, filtered, and the collected solids were dried under vacuum to afford ethyl 2-oxo-2-(((5-phenylpyrimidin-2-yl)methyl)amino)acetate. ¹H NMR (400 MHz, chloroform-d) δ 8.93 (s, 2H), 7.58-7.47 (m, 5H), 4.84-4.83 (m, 2H), 4.43-4.38 (q, J=7.2 Hz, 2H), 1.43-1.40 (t, J=7.2 Hz, 3H).

### Step D: ethyl 2-(allyl((5-phenylpyrimidin-2-yl)methyl)amino)-2-oxoacetate

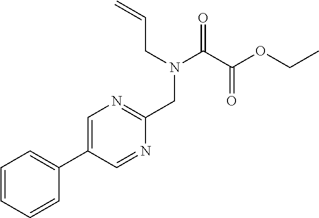 [SMILES: C=CCN(Cc1ncc(-c2ccccc2)cn1)C(=O)C(=O)OCC]

Allyl bromide (19.0 g, 0.157 mol) was added dropwise to a mixture of ethyl 2-oxo-2-(((5-phenylpyrimidin-2-yl)methyl)amino)acetate (18.0 g, 0.063 mol) and cesium carbonate (61 g, 0.19 mol) in DMF (100 mL) at room temperature under a nitrogen atmosphere. The reaction mixture was stirred at room temperature for 12 hours. The reaction mixture was poured into ice water (1.0 L) and extracted with ethyl acetate (3×1.0 L). The organic layers were separated, dried over anhydrous sodium sulfate, filtered, and concentrated under reduced pressure. The residue was purified by silica gel chromatography (eluting ethyl acetate in petroleum ether) to afford ethyl 2-(allyl((5-phenylpyrimidin-2-yl)methyl)amino)-2-oxoacetate. ¹H NMR (400 MHz, chloroform-d) δ 8.90 (s, 2H), 7.56-7.51 (m, 5H), 5.90-5.80 (m, 1H), 5.27-5.20 (m, 2H), 4.86-4.85 (m, 2H), 4.38-4.28 (m, 2H), 4.26-4.11 (m, 2H), 1.42-1.25 (m, 3H). LC/MS (m/z): 326 (M+H)⁺

### Step E: ethyl 2-oxo-2-((2-oxoethyl)((5-phenylpyrimidin-2-yl)methyl)amino)acetate

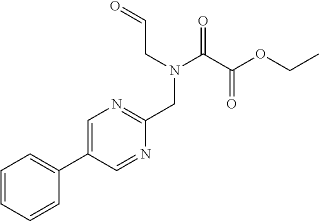 [SMILES: CCOC(=O)C(=O)N(CC=O)Cc1ncc(-c2ccccc2)cn1]

A mixture of ethyl 2-(allyl((5-phenylpyrimidin-2-yl)methyl)amino)-2-oxoacetate (1.1 g, 3.4 mmol) in DCM (35 ml) was sparged with a stream of ozone gas for 40 minutes at −78° C. The mixture was then sparged with a stream of oxygen gas at −78° C. The reaction mixture was warmed to room temperature and treated with dimethyl sulfide (4.94 mL, 67.6 mmol). The reaction mixture was then stirred at room temperature for an additional 2 hours. The mixture was filtered, and the filtrate was concentrated under reduced pressure to afford ethyl 2-oxo-2-((2-oxoethyl)((5-phenylpyrimidin-2-yl)methyl)amino)acetate, which was used without purification in the next step. LC/MS (m/z): 328 (M+H)⁺

Examples shown in Intermediate Table 15 below, were prepared according to procedures analogous to those outlined in Intermediate 136 above using the appropriate starting materials, described in the Preparations or Intermediates above, or as obtained from commercial sources.

Intermediate Table 15

| Intermediate | Structure | Name | Mass [M + H]⁺ |
|---|---|---|---|
| Intermediate 137 | 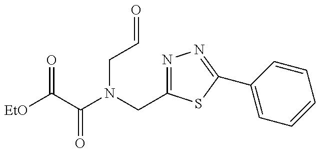 [SMILES: CCOC(=O)C(=O)N(CC=O)Cc1nnc(-c2ccccc2)s1] | ethyl 2-oxo-2-((2-oxoethyl)((5- phenyl-1,3,4-thiadiazol-2- yl)methyl)amino)acetate | 334 |

### Intermediate 138

### Preparation of 1-cyclopentyl-4-((5-(4,4,5,5-tetramethyl-1,3,2-dioxaborolan-2-yl)pyrimidin-2-yl)methyl)piperazine-2,3-dione

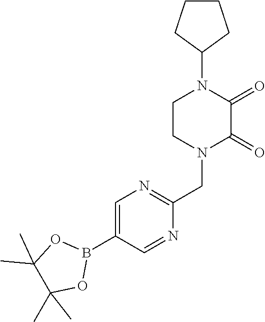 [SMILES: CC1(C)OB(c2cnc(CN3CCN(C4CCCC4)C(=O)C3=O)nc2)OC1(C)C]

A mixture of 1-((5-bromopyrimidin-2-yl)methyl)-4-cyclopentylpiperazine-2,3-dione (0.830 g, 2.35 mmol), bis(pinacolato)diboron (1190 mg, 4.70 mmol), tetrakis(triphenylphosphine)palladium(0) (272 mg, 0.235 mmol), and potassium acetate (692 mg, 7.05 mmol) in 1,4-dioxane (32 mL) was sparged with a stream of nitrogen for 5 minutes. The reaction mixture was then stirred and heated to 105° C. for 2 hours. The reaction mixture was cooled to room temperature, filtered through Celite, diluted with water (150 mL), and washed with petroleum ether (3×100 mL) and then EtOAc (2×100 mL). The aqueous layer was partially concentrated under reduced pressure to remove residual organic solvents, and then lyophilized to afford 1-cyclopentyl-4-((5-(4,4,5,5-tetramethyl-1,3,2-dioxaborolan-2-yl)pyrimidin-2-yl)methyl)piperazine-2,3-dione. ¹H NMR (400 MHz, methanol-d₄) δ 8.85 (s, 2H), 4.90-4.89 (m, 1H), 4.85 (s, 2H), 3.78-3.70 (m, 2H), 3.68-3.63 (m, 2H), 1.96-1.87 (m, 2H), 1.84-1.74 (m, 2H), 1.70-1.62 (m, 4H), 1.27 (s, 12H).

### Intermediate 139

### Preparation of 1-(3-phenylcyclobutyl)piperazine-2,3-dione

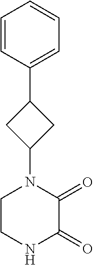 [SMILES: O=C1NCCN(C2CC(c3ccccc3)C2)C1=O]

### Step A: tert-butyl (2-((3-phenylcyclobutyl)amino)ethyl)carbamate

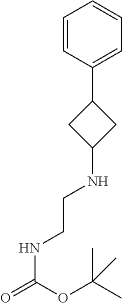 [SMILES: CC(C)(C)OC(=O)NCCNC1CC(c2ccccc2)C1]

Sodium triacetoxyborohydride (2.90 g, 13.7 mmol) was added to a mixture of 3-phenylcyclobutan-1-one (1.00 g, 6.84 mmol) and tert-butyl (2-aminoethyl)carbamate (1.21 g, 7.52 mmol) in dichloromethane (40 mL) at 0° C. The reaction mixture was warmed to room temperature and stirred for 3 days. The reaction mixture was quenched with saturated aqueous sodium bicarbonate (50 mL) and then diluted with ethyl acetate (250 mL) and water (50 mL). The organic layer was separated, and the aqueous layer was washed with additional ethyl acetate (2×50 mL). The organic layers were combined, washed with brine (25 mL), dried over magnesium sulfate, filtered, and concentrated under reduced pressure. The residue was dissolved in dichloromethane (20 mL), and the mixture was stirred at room temperature. Hexanes (80 mL) were added dropwise via addition funnel to the mixture over a period of 15 minutes. The mixture was stirred for 4 days. The mixture was filtered, and the collected solids were washed with additional hexanes (20 mL) and then dried under vacuum to afford tert-butyl (2-((3-phenylcyclobutyl)amino)ethyl)carbamate as a mixture of cis/trans isomers. LC/MS (m/z): 291 (M+H)⁺

### Step B: N¹-(3-phenylcyclobutyl)ethane-1,2-diamine

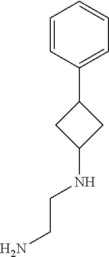 [SMILES: NCCNC1CC(c2ccccc2)C1]

TFA (5.64 mL, 73.2 mmol) was added to a mixture of tert-butyl (2-((3-phenylcyclobutyl)amino)ethyl)carbamate (0.850 g, 2.93 mmol) in dichloromethane (20 mL) at room temperature. The reaction mixture was stirred for 1 hour at room temperature. The reaction mixture was concentrated under reduced pressure and dried under vacuum to afford N¹-(3-phenylcyclobutyl)ethane-1,2-diamine as a bis-TFA salt and a mixture of cis/trans isomers. LC/MS (m/z): 191 (M+H)⁺

### Step C: 1-(3-phenylcyclobutyl)piperazine-2,3-dione

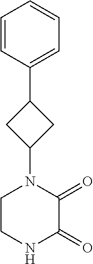 [SMILES: O=C1NCCN(C2CC(c3ccccc3)C2)C1=O]

Diethyl oxalate (0.397 mL, 2.93 mmol) was added to a mixture of N¹-(3-phenylcyclobutyl)ethane-1,2-diamine bis(2,2,2-trifluoroacetate) (1220 mg, 2.93 mmol) and triethylamine (1.63 mL, 11.7 mmol) in ethanol (10 mL) at room temperature. The reaction mixture was heated to 80° C. and stirred for 6 days under a reflux condenser. The reaction mixture was cooled to room temperature and concentrated under reduced pressure. The residue was purified by silica gel chromatography (eluting [1:3 ethanol:ethyl acetate] in dichloromethane) to afford 1-(3-phenylcyclobutyl)piperazine-2,3-dione as a mixture of cis/trans isomers. LC/MS (m/z): 245 (M+H)⁺

### Intermediate 140

### Preparation of mixture of 1-((5-bromo-1,3,4-thiadiazol-2-yl)methyl)-4-cyclobutylpiperazine-2,3-dione and 1-((5-bromo-1,3,4-thiadiazol-2-yl)methyl)-4-cyclobutyl-1,4-dihydropyrazine-2,3-dione

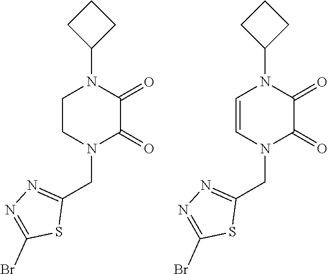 [SMILES: O=C1C(=O)N(C2CCC2)CCN1Cc1nnc(Br)s1 ; O=c1c(=O)n(C2CCC2)ccn1Cc1nnc(Br)s1]

### Step A: 2-bromo-5-(bromomethyl)-1,3,4-thiadiazole

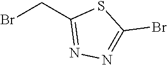

N-Bromosuccinimide (0.994 g, 5.59 mmol) and azobisisobutyronitrile (0.092 g, 0.56 mmol) were added to a mixture of 2-bromo-5-methyl-1,3,4-thiadiazole (1.0 g, 5.6 mmol) in carbon tetrachloride (20 mL) under an inert atmosphere at 20° C. The mixture was then heated to 80° C. and stirred for 4 hours. The reaction mixture was filtered, and the filtrate was concentrated under reduced pressure. The residue was diluted with water (35 mL) and extracted with DCM (2×25 mL). The organic layers were combined, dried over sodium sulfate, filtered, and concentrated under reduced pressure. The residue was purified by silica gel chromatography (eluting EtOAc/hexanes) to afford 2-bromo-5-(bromomethyl)-1,3,4-thiadiazole. LC/MS (m/z): 259 (M+H)⁺

### Step B: N-((5-bromo-1,3,4-thiadiazol-2-yl)methyl)prop-2-en-1-amine

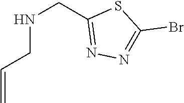

Potassium carbonate (2.97 g, 21.5 mmol) was added to a mixture of prop-2-en-1-amine (1.41 mL, 18.7 mmol) and 2-bromo-5-(bromomethyl)-1,3,4-thiadiazole (1.85 g, 7.17 mmol) in THE (50 mL) at room temperature. The reaction mixture was stirred at room temperature for 13 hours. The mixture was filtered, and the filtrate was concentrated under reduced pressure to afford N-((5-bromo-1,3,4-thiadiazol-2-yl)methyl)prop-2-en-1-amine, which was used without purification. LC/MS (m/z): 233, 235 (M+H)⁺

### Step C: ethyl 2-(allyl((5-bromo-1,3,4-thiadiazol-2-yl)methyl)amino)-2-oxoacetate

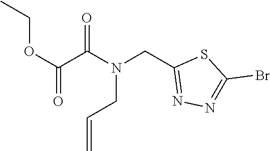 [SMILES: C=CCN(Cc1nnc(Br)s1)C(=O)C(=O)OCC]

Ethyl 2-chloro-2-oxoacetate (1.15 mL, 10.8 mmol) was added to a mixture of N-((5-bromo-1,3,4-thiadiazol-2-yl)methyl)prop-2-en-1-amine (1.68 g, 7.17 mmol) and TEA (3.00 mL, 21.5 mmol) in DCM (30 mL) at 0° C. The reaction was stirred at room temperature for 1 hour. The mixture was diluted with DCM (30 mL) and water (50 mL). The organic layer was separated, washed with brine (30 mL), dried over Na₂SO₄, filtered, and concentrated under reduced pressure. The residue was purified by silica gel chromatography (eluting ethyl acetate in petroleum ether) to afford ethyl 2-(allyl((5-bromo-1,3,4-thiadiazol-2-yl)methyl)amino)-2-oxoacetate. ¹H NMR (400 MHz, chloroform-d) δ 5.87-5.68 (m, 1H), 5.39-5.24 (m, 2H), 4.86 (s, 2H), 4.40-4.29 (m, 2H), 4.06-3.96 (m, 2H), 1.44-1.32 (m, 3H). LC/MS (m/z): 334, 336 (M+H)⁺

### Step D: ethyl 2-(((5-bromo-1,3,4-thiadiazol-2-yl)methyl)(2-oxoethyl)amino)-2-oxoacetate

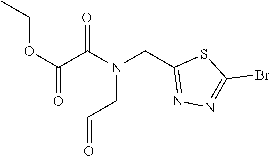 [SMILES: CCOC(=O)C(=O)N(CC=O)Cc1nnc(Br)s1]

Osmium tetroxide (11 mg, 0.042 mmol) was added to a mixture of ethyl 2-(allyl((5-bromo-1,3,4-thiadiazol-2-yl)methyl)amino)-2-oxoacetate (0.140 g, 0.419 mmol) and sodium periodate (0.300 g, 1.40 mmol) in THE (3 mL) at 0° C. The reaction was stirred at room temperature for 1.5 hours. The mixture was quenched with saturated aqueous sodium bisulfite and stirred for 15 minutes. The mixture was then diluted with EtOAc (30 mL) and water (50 mL). The organic layer was separated, washed with brine (30 mL), dried over Na₂SO₄, filtered, and concentrated under reduced pressure to afford ethyl 2-(((5-bromo-1,3,4-thiadiazol-2-yl)methyl)(2-oxoethyl)amino)-2-oxoacetate which was used without purification in the next step. LC/MS (m/z): 336, 338 (M+H)⁺

### Step E: mixture of 1-((5-bromo-1,3,4-thiadiazol-2-yl)methyl)-4-cyclobutylpiperazine-2,3-dione and 1-((5-bromo-1,3,4-thiadiazol-2-yl)methyl)-4-cyclobutyl-1,4-dihydropyrazine-2,3-dione

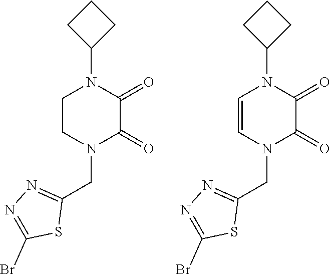 [SMILES: O=C1C(=O)N(C2CCC2)CCN1Cc1nnc(Br)s1 ; O=c1c(=O)n(C2CCC2)ccn1Cc1nnc(Br)s1]

Sodium triacetoxyborohydride (128 mg, 0.602 mmol) was added to a mixture of ethyl 2-(((5-bromo-1,3,4-thiadiazol-2-yl)methyl)(2-oxoethyl)amino)-2-oxoacetate (135 mg, 0.402 mmol) and cyclobutanamine (31 mg, 0.44 mmol) in DCE (5 mL) at room temperature. The mixture was stirred at room temperature for 2 hours. The reaction mixture was concentrated under reduced pressure. The residue was purified by silica gel chromatography (eluting methanol in dichloromethane) to afford 1-((5-bromo-1,3,4-thiadiazol-2-yl)methyl)-4-cyclobutylpiperazine-2,3-dione, which contained the alkene by-product 1-((5-bromo-1,3,4-thiadiazol-2-yl)methyl)-4-cyclobutyl-1,4-dihydropyrazine-2,3-dione. The mixture was used without purification.

### Intermediate 141

### Preparation of 1-(bicyclo[1.1.1]pentan-1-yl)-4-((5-bromo-1,3,4-thiadiazol-2-yl)methyl)piperazine-2,3-dione

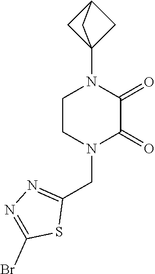 [SMILES: O=C1C(=O)N(C23CC(C2)C3)CCN1Cc1nnc(Br)s1]

Sodium triacetoxyborohydride (132 mg, 0.625 mmol) was added to a mixture of ethyl 2-(((5-bromo-1,3,4-thiadiazol-2-yl)methyl)(2-oxoethyl)amino)-2-oxoacetate (140 mg, 0.42 mmol) and bicyclo[1.1.1]pentan-1-amine hydrochloride (0.050 g, 0.42 mmol) in DCE (5 mL) at room temperature. The mixture was stirred at room temperature for 2 hours. The reaction mixture was concentrated under reduced pressure. The residue was purified by silica gel chromatography (eluting methanol in dichloromethane) to afford 1-(bicyclo[1.1.1]pentan-1-yl)-4-((5-bromo-1,3,4-thiadiazol-2-yl)methyl)piperazine-2,3-dione. LC/MS (m/z): 357, 359 (M+H)⁺

Examples shown in Intermediate Table 16 below, were prepared according to procedures analogous to those outlined in Intermediate 141 above using the appropriate starting materials, described in the Preparations or Intermediates above, or as obtained from commercial sources.

Intermediate Table 16

| Intermediate | Structure | Name | Mass [M + H]⁺ |
|---|---|---|---|
| Intermediate 142 | 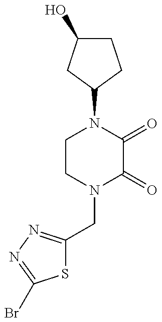 | (cis)-1-((5-bromo-1,3,4- thiadiazol-2-yl)methyl)-4-(3- hydroxycyclopentyl) piperazine-2,3-dione | 375, 377 |

### Intermediate 143

### Preparation of 1-((5-bromo-1,3,4-thiadiazol-2-yl)methyl)-4-cyclopentyl-1,4-dihydropyrazine-2,3-dione

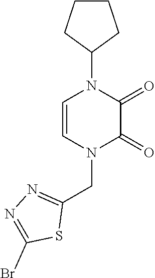 [SMILES: O=c1c(=O)n(C2CCCC2)ccn1Cc1nnc(Br)s1]

A mixture of ethyl 2-(((5-bromo-1,3,4-thiadiazol-2-yl)methyl)(2-oxoethyl)amino)-2-oxoacetate (0.150 g, 0.446 mmol) and cyclopentanamine (38 mg, 0.45 mmol) in DCE (10 mL) was stirred at room temperature for 12 hours. The reaction mixture was concentrated under reduced pressure. The residue was purified by reverse phase HPLC (eluting acetonitrile in water, with ammonium bicarbonate modifier) to afford 1-((5-bromo-1,3,4-thiadiazol-2-yl)methyl)-4-cyclopentyl-1,4-dihydropyrazine-2,3-dione. LC/MS (m/z): 357, 359 (M+H)⁺

Examples shown in Intermediate Table 17 below, were prepared according to procedures analogous to those outlined in Intermediate 143 above using the appropriate starting materials, described in the Preparations or Intermediates above, or as obtained from commercial sources.

Intermediate Table 17

| Intermediate | Structure | Name | Mass [M + H]⁺ |
|---|---|---|---|
| Intermediate 144 | 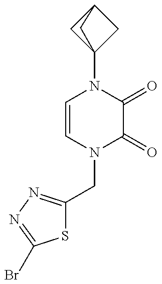 [SMILES: O=c1c(=O)n(C23CC(C2)C3)ccn1Cc1nnc(Br)s1] | 1-(bicyclo[1.1.1 ]pentan-1-yl)- 4-((5-bromo-1,3,4-thiadiazol- 2-yl)methyl)-1,4- dihydropyrazine-2,3-dione | 355, 357 |
| Intermediate 145 | 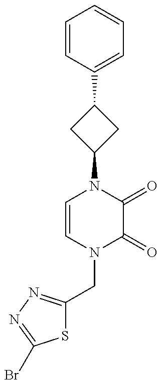 [SMILES: O=c1c(=O)n([C@H]2C[C@H](c3ccccc3)C2)ccn1Cc1nnc(Br)s1] | 1-((5-bromo-1,3,4-thiadiazol- 2-yl)methyl)-4-((trans)-3- phenylcyclobutyl)-1,4- dihydropyrazine-2,3-dione | 419, 421 |
| Intermediate 146 | 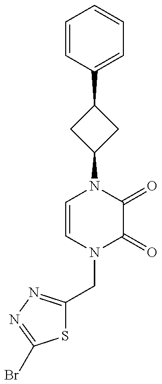 [SMILES: O=c1c(=O)n([C@H]2C[C@@H](c3ccccc3)C2)ccn1Cc1nnc(Br)s1] | 1-((5-bromo-1,3,4-thiadiazol- 2-yl)methyl)-4-((cis)-3- phenylcyclobutyl)-1,4- dihydropyrazine-2,3-dione | 419, 421 |
| Intermediate 147 | 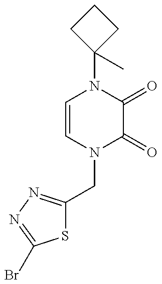 [SMILES: CC1(n2ccn(Cc3nnc(Br)s3)c(=O)c2=O)CCC1] | 1-((5-bromo-1,3,4-thiadiazol- 2-yl)methyl)-4-(1- methylcyclobutyl)-1,4- dihydropyrazine-2,3-dione | 357, 359 |

### Intermediate 148

### Preparation of 6-((2-ethoxy-2-oxo-N-(2-oxoethyl)acetamido)methyl)-3-phenylpyridazine 1-oxide

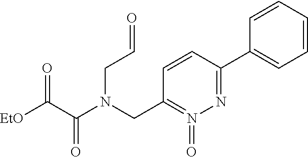 [SMILES: CCOC(=O)C(=O)N(CC=O)Cc1ccc(-c2ccccc2)n[n+]1[O-]]

### Step A: 3-methyl-6-phenylpyridazine

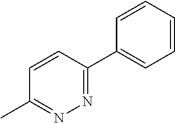

A mixture of 3-chloro-6-methylpyridazine (5.0 g, 39 mmol), phenylboronic acid (4.74 g, 38.9 mmol), potassium phosphate tribasic (17 g, 78 mmol) and Pd(dppf)Cl₂ (2.28 g, 3.11 mmol) in 1,4-dioxane (40 mL) and water (8.0 mL) was sparged with nitrogen at room temperature. The mixture was stirred and heated 80° C. for 4 hours. The reaction mixture was cooled to room temperature and quenched with saturated aqueous NH₄Cl (50 mL). The mixture was extracted with EtOAc (3×50 mL). The organic layers were combined, washed with brine (15 mL), dried over anhydrous Na₂SO₄, filtered, and concentrated under reduced pressure. The residue was purified by silica gel chromatography (eluting ethyl acetate in petroleum ether) to afford 3-methyl-6-phenylpyridazine. LC/MS (m/z): 171 (M+H)⁺

### Step B: 3-(chloromethyl)-6-phenylpyridazine

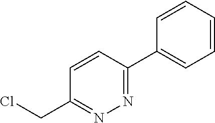 [SMILES: ClCc1ccc(-c2ccccc2)nn1]

1,3,5-trichloro-1,3,5-triazinane-2,4,6-trione (2.84 g, 12.2 mmol) was added to a mixture of 3-methyl-6-phenylpyridazine (5.2 g, 31 mmol) in trichloromethane (200 mL) at 20° C. The mixture was stirred and heated at 60° C. for 12 hours. The mixture was cooled to room temperature, filtered, and concentrated under reduced pressure. The residue was purified by silica gel chromatography (eluting ethyl acetate in petroleum ether) to afford 3-(chloromethyl)-6-phenylpyridazine. ¹H NMR (400 MHz, methanol-d₄) δ 8.82 (br dd, J=5.7, 8.4 Hz, 1H), 8.68-8.49 (m, 1H), 8.11 (dd, J=1.4, 8.0 Hz, 2H), 7.80-7.65 (m, 3H), 5.08 (s, 2H). LC/MS (m/z): 205 (M+H)⁺

### Step C: N-((6-phenylpyridazin-3-yl)methyl)prop-2-en-1-amine

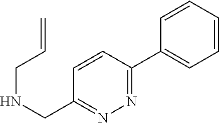 [SMILES: C=CCNCc1ccc(-c2ccccc2)nn1]

Prop-2-en-1-amine (2.0 mL, 27 mmol) was added to a mixture of 3-(chloromethyl)-6-phenylpyridazine hydrochloride (3.0 g, 12 mmol) and potassium carbonate (5.16 g, 37.3 mmol) in DMF (40 mL) at 20° C. The mixture was stirred and heated at 40° C. for 12 hours. The reaction mixture was cooled to room temperature and concentrated under reduced pressure to afford ethyl 2-(allyl((6-phenylpyridazin-3-yl)methyl)amino)-2-oxoacetate which was used without purification in the next step. LC/MS (m/z): 226 (M+H)⁺

### Step D: ethyl 2-(allyl((6-phenylpyridazin-3-yl)methyl)amino)-2-oxoacetate

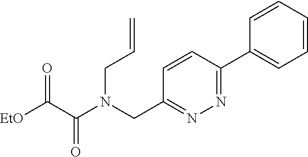 [SMILES: C=CCN(Cc1ccc(-c2ccccc2)nn1)C(=O)C(=O)OCC]

Ethyl oxalyl chloride (2.1 mL, 19 mmol) was added to a solution of N-((6-phenylpyridazin-3-yl)methyl)prop-2-en-1-amine (2.8 g, 12 mmol) and potassium carbonate (3.44 g, 24.9 mmol) in DMF (20 mL) at 0° C. The mixture was stirred at room temperature for 3 hours. The reaction mixture was quenched with water (400 mL) and extracted with EtOAc (3×100 mL). The organic layers were combined, washed with brine (400 mL), dried over anhydrous Na₂SO₄, filtered, and concentrated under reduced pressure. The residue was purified by silica gel chromatography (eluting ethyl acetate in petroleum ether) to afford ethyl 2-(allyl((6-phenylpyridazin-3-yl)methyl)amino)-2-oxoacetate. ¹H NMR (500 MHz, methanol-d₄) δ 8.20-8.14 (m, 1H), 8.10-8.03 (m, 2H), 7.86-7.68 (m, 1H), 7.59-7.48 (m, 3H), 5.98-5.72 (m, 1H), 5.38-5.18 (m, 2H), 4.95-4.89 (m, 2H), 4.41-4.29 (m, 2H), 4.17-4.04 (m, 2H), 1.35 (t, J=7.1 Hz, 2H), 1.28 (t, J=7.1 Hz, 1H). LC/MS (m/z): 326 (M+H)⁺

### Step E: 6-((N-allyl-2-ethoxy-2-oxoacetamido)methyl)-3-phenylpyridazine 1-oxide

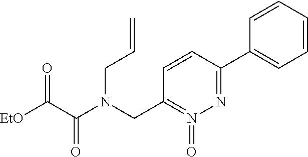 [SMILES: C=CCN(Cc1ccc(-c2ccccc2)n[n+]1[O-])C(=O)C(=O)OCC]

meta-Chloroperoxybenzoic acid (66 mg, 0.31 mmol, 85%) was added to a mixture of ethyl 2-(allyl((6-phenylpyridazin-3-yl)methyl)amino)-2-oxoacetate (0.100 g, 0.307 mmol) in DCM (4 mL) at 0° C. The mixture was stirred for 1 hour at room temperature. The mixture was concentrated under reduced pressure to afford 6-((N-allyl-2-ethoxy-2-oxoacetamido)methyl)-3-phenylpyridazine 1-oxide which was used without purification in the next step. ¹H NMR (400 MHz, chloroform-d) δ 8.10 (d, J=1.6 Hz, 1H), 8.04-7.93 (m, 2H), 7.85-7.70 (m, 1H), 7.60-7.39 (m, 3H), 6.00-5.71 (m, 1H), 5.42-5.18 (m, 2H), 4.76 (s, 2H), 4.43-4.27 (m, 2H), 4.26-4.06 (m, 2H), 1.46-1.19 (m, 3H). LC/MS (m/z): 342 (M+H)⁺

### Step F: 6-((2-ethoxy-2-oxo-N-(2-oxoethyl)acetamido)methyl)-3-phenylpyridazine 1-oxide

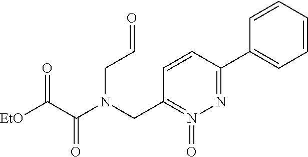 [SMILES: CCOC(=O)C(=O)N(CC=O)Cc1ccc(-c2ccccc2)n[n+]1[O-]]

Sodium periodate (188 mg, 0.879 mmol) and osmium(VIII) oxide (7 mg, 0.03 mmol) were added to a mixture of 3-((N-allyl-2-ethoxy-2-oxoacetamido)methyl)-6-phenylpyridazine 1-oxide (0.100 g, 0.293 mmol) in water (3 mL) and THE (3 mL) at 0° C. The mixture was stirred for 1 hour at room temperature. The reaction mixture was quenched with saturated aqueous Na₂SO₃ (10 mL). The mixture was diluted with EtOAc (30 mL) and water (50 mL). The organic layer was separated, washed with brine (30 mL), dried over anhydrous sodium sulfate, filtered, and concentrated under reduced pressure to afford 3-((2-ethoxy-2-oxo-N-(2-oxoethyl)acetamido)methyl)-6-phenylpyridazine 1-oxide which was used in the next step without purification. LC/MS (m/z): 344 (M+H)⁺

### Intermediates 149 and 150

### Preparation of 1-((6-chloropyridazin-3-yl)methyl)-4-((trans)-3-phenylcyclobutyl)piperazine-2,3-dione and 1-((6-chloropyridazin-3-yl)methyl)-4-((cis)-3-phenylcyclobutyl)piperazine-2,3-dione

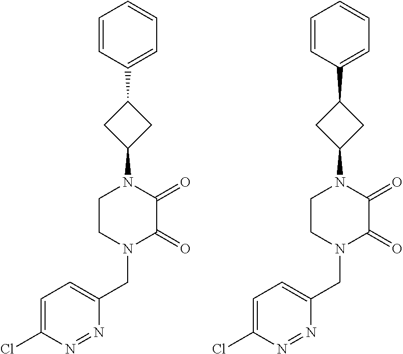 [SMILES: O=C1C(=O)N([C@H]2C[C@H](c3ccccc3)C2)CCN1Cc1ccc(Cl)nn1 ; O=C1C(=O)N([C@H]2C[C@@H](c3ccccc3)C2)CCN1Cc1ccc(Cl)nn1]

1-((6-chloropyridazin-3-yl)methyl)-4-(3-phenylcyclobutyl)piperazine-2,3-dione (0.400, 1.08 mmol) was resolved by Chiral-SFC (Daicel Chiralcel OD-H (250 mm×30 mm, Sum); eluting 40% EtOH in CO₂ with 0.1% aqueous ammonia) to give 1-((6-chloropyridazin-3-yl)methyl)-4-((trans)-3-phenylcyclobutyl)piperazine-2,3-dione (tᵣ=1.112 min) as the first eluting peak and 1-((6-chloropyridazin-3-yl)methyl)-4-((cis)-3-phenylcyclobutyl)piperazine-2,3-dione (tᵣ=2.364 min) as the second eluting peak.

Peak 1: ¹H NMR (400 MHz, methanol-d₄) δ 7.84-7.73 (m, 2H), 7.36-7.30 (m, 4H), 7.23-7.17 (m, 1H), 5.13-5.05 (m, 1H), 4.95 (s, 2H), 3.82 (s, 4H), 3.59-3.51 (m, 1H), 2.84-2.73 (m, 2H), 2.46 (ddd, J=3.9, 8.8, 13.1 Hz, 2H). LC/MS (m/z): 371 (M+H)⁺

Peak 2: ¹H NMR (400 MHz, methanol-d₄) δ 7.82-7.73 (m, 2H), 7.33-7.24 (m, 4H), 7.21-7.14 (m, 1H), 4.94 (s, 2H), 4.84-4.73 (m, 1H), 3.81-3.69 (m, 4H), 3.29-3.18 (m, 1H), 2.70-2.60 (m, 2H), 2.40-2.27 (m, 2H). LC/MS (m/z): 371 (M+H)⁺

### Intermediate 151

### Preparation of 1-((cis)-bicyclo[3.1.0]hexan-3-yl)-4-((5-bromo-3-fluoropyridin-2-yl)methyl)piperazine-2,3-dione

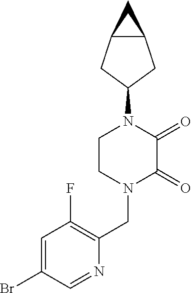 [SMILES: O=C1C(=O)N([C@H]2C[C@H]3C[C@H]3C2)CCN1Cc1ncc(Br)cc1F]

### Step A: 5-bromo-2-(bromomethyl)-3-fluoropyridine

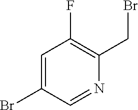

A mixture of (5-bromo-3-fluoropyridin-2-yl)methanol (0.950 g, 4.61 mmol), carbon tetrabromide (2.30 g, 6.92 mmol), and triphenylphosphine (1.81 g, 6.92 mmol) in DCM (25 mL) was stirred at room temperature for 16 hours. The mixture was concentrated under reduced pressure, and the residue was purified by silica gel chromatography (eluting ethyl acetate in petroleum ether) to afford 5-bromo-2-(bromomethyl)-3-fluoropyridine. LC/MS (m/z): 270 (M+H)⁺

### Step B: ethyl 2-(allyl((5-bromo-3-fluoropyridin-2-yl)methyl)amino)-2-oxoacetate

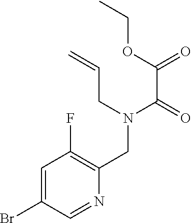 [SMILES: C=CCN(Cc1ncc(Br)cc1F)C(=O)C(=O)OCC]

NaH (67 mg, 1.7 mmol, 60% dispersion in mineral oil) was added to a mixture of ethyl 2-(allylamino)-2-oxoacetate (175 mg, 1.11 mmol) in DMF (5 mL) at 0° C. The mixture was warmed to room temperature and stirred for 30 minutes. 5-bromo-2-(bromomethyl)-3-fluoropyridine (299 mg, 1.11 mmol) was added to the mixture. The mixture was stirred at room temperature for 3 hours. The mixture was concentrated under reduced pressure, and the residue was purified by silica gel chromatography (eluting ethyl acetate in petroleum ether) to afford ethyl 2-(allyl((5-bromo-3-fluoropyridin-2-yl)methyl)amino)-2-oxoacetate. LC/MS (m/z): 345, 347 (M+H)⁺

### Step C: ethyl 2-(((5-bromo-3-fluoropyridin-2-yl)methyl)(2-oxoethyl)amino)-2-oxoacetate

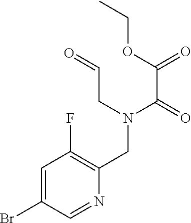 [SMILES: CCOC(=O)C(=O)N(CC=O)Cc1ncc(Br)cc1F]

Osmium tetroxide (9 mg, 0.04 mmol) was added to a mixture of ethyl 2-(allyl((5-bromo-3-fluoropyridin-2-yl)methyl)amino)-2-oxoacetate (110 mg, 0.32 mmol) and sodium periodate (204 mg, 0.956 mmol) in THE (2 ml) at 0° C. The reaction was stirred at room temperature for 2 hours. The mixture was quenched with saturated aqueous Na₂SO₃ (30 mL) and diluted with EtOAc (15 mL) and water (10 mL). The organic layer was separated, washed with brine (5 mL), dried over sodium sulfate, filtered, and concentrated under reduced pressure to afford ethyl 2-(((5-bromo-3-fluoropyridin-2-yl)methyl)(2-oxoethyl)amino)-2-oxoacetate which was used without purification in the next step. LC/MS (m/z): 347, 349 (M+H)⁺

### Step D: 1-((cis)-bicyclo[3.1.0]hexan-3-yl)-4-((5-bromo-3-fluoropyridin-2-yl)methyl)piperazine-2,3-dione

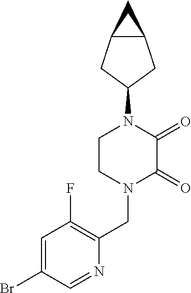 [SMILES: O=C1C(=O)N([C@H]2C[C@H]3C[C@H]3C2)CCN1Cc1ncc(Br)cc1F]

A mixture of ethyl 2-(((5-bromo-3-fluoropyridin-2-yl)methyl)(2-oxoethyl)amino)-2-oxoacetate (50 mg, 0.14 mmol), triethylamine (0.020 mL, 0.14 mmol) and bicyclo[3.1.0]hexan-3-ammonium iodide (33 mg, 0.15 mmol) in DCE (1.5 mL) was stirred at room temperature for 16 hours. The reaction mixture was concentrated under reduced pressure, and the residue was purified by silica gel chromatography (eluting methanol in dichloromethane) to afford 1-((cis)-bicyclo[3.1.0]hexan-3-yl)-4-((5-bromo-3-fluoropyridin-2-yl)methyl)piperazine-2,3-dione. LC/MS (m/z): 382, 384 (M+H)⁺

### Intermediate 152

### Preparation of 1-((5-bromopyridin-2-yl)methyl)-4-cyclobutyl-1,4-dihydropyrazine-2,3-dione

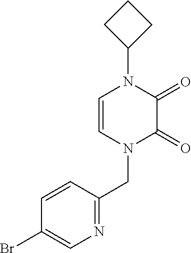 [SMILES: O=c1c(=O)n(C2CCC2)ccn1Cc1ccc(Br)cn1]

A mixture of 1-cyclobutyl-1,4-dihydropyrazine-2,3-dione (1.40 g, 8.42 mmol), (5-bromopyridin-2-yl)methyl methanesulfonate (3.14 g, 11.8 mmol), and potassium carbonate (1.75 g, 12.6 mmol) in acetonitrile (20 mL) was heated to 50° C. and stirred for 24 hours. The mixture was cooled to room temperature and diluted with dichloromethane (100 mL). The suspension was stirred at room temperature for 30 minutes and then filtered through Celite. The filtrate was concentrated under reduced pressure, and the residue was purified by silica gel chromatography (eluting with [3:1 ethyl acetate:ethanol] in dichloromethane) to afford 1-((5-bromopyridin-2-yl)methyl)-4-cyclobutyl-1,4-dihydropyrazine-2,3-dione. ¹H NMR (499 MHz, DMSO-d₆) δ 8.65 (d, J=2.0 Hz, 1H), 8.04 (dd, J=8.4, 2.4 Hz, 1H), 7.33 (d, J=8.4 Hz, 1H), 6.80 (d, J=6.2 Hz, 1H), 6.71 (d, J=6.2 Hz, 1H), 5.00 (s, 2H), 4.91 (p, J=8.6 Hz, 1H), 2.30-2.20 (m, 4H), 1.78-1.70 (m, 2H). LC/MS (m/z): 336, 338 (M+H)⁺

### Intermediate 153

### Preparation of 4-(chloromethyl)-1-phenyl-1H-imidazole

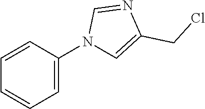 [SMILES: ClCc1cn(-c2ccccc2)cn1]

Sulfurous dichloride (0.025 mL, 0.34 mmol) was added to a mixture of (1-phenyl-1H-imidazol-4-yl)methanol (30 mg, 0.17 mmol) in DCM (2 mL) at 0° C. The mixture was stirred for 1.5 hours. The mixture was filtered and the filtrate was concentrated under reduced pressure to afford 4-(chloromethyl)-1-phenyl-1H-imidazole, which was used without purification in the next step. LC/MS (m/z): 193 (M+H)⁺

### Intermediate 154

### Preparation of 2-(bromomethyl)-5-(2H-tetrazol-2-yl)pyridine

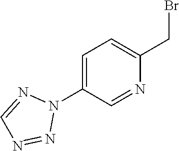 [SMILES: BrCc1ccc(-n2ncnn2)cn1]

### Step A: 2-methyl-5-((tetrafluoro-λ⁵-boranyl)diazenyl)pyridine

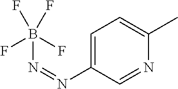

A mixture of 6-methylpyridin-3-amine (0.500 g, 4.62 mmol) and trifluoroborane hydrofluoride (1.50 mL, 9.43 mmol) in ethanol (10 mL) was degassed and backfilled with nitrogen (3×) at 0° C. After stirring for 10 minutes at 0° C., tert-butyl nitrite (0.890 mL, 9.48 mmol) was added to the mixture at 0° C. The mixture was stirred for an additional 6 hours at room temperature.

The reaction was filtered and the collected solids were washed with EtOAc (2×10 mL) to afford 2-methyl-5-((tetrafluoro-λ⁵-boraneyl)diazenyl)pyridine, which was used without purification in the next step.

### Step B: 2-methyl-5-(2H-tetrazol-2-yl)pyridine

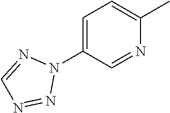

Silver (I) trifluoromethanesulfonate (745 mg, 2.90 mmol) was added to a mixture of 2-methyl-5-((tetrafluoro-λ⁵-boraneyl)diazenyl)pyridine (0.500 g, 2.42 mmol) in THE (15 mL) at −78° C. and then stirred for 10 minutes. The mixture was warmed to room temperature, and triethylamine (0.51 mL, 3.6 mmol) was added. The mixture was stirred for an additional 10 hours. A mixture of CsF (1.10 g, 7.25 mmol) in MeOH (2 mL) was added and the mixture was stirred for 6 hours at room temperature. The mixture was filtered and the filtrate was concentrated under reduced pressure to afford 2-methyl-5-(2H-tetrazol-2-yl)pyridine, which was used in the next step without purification. LC/MS (m/z): 162 (M+H)⁺

### Step C: 2-(bromomethyl)-5-(2H-tetrazol-2-yl)pyridine

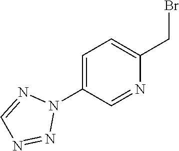 [SMILES: BrCc1ccc(-n2ncnn2)cn1]

A mixture of 2-methyl-5-(2H-tetrazol-2-yl)pyridine (0.050 g, 0.31 mmol), NBS (28 mg, 0.16 mmol), and AIBN (6 mg, 0.04 mmol) in DMF (1 ml) was stirred at 80° C. for 12 hours. The mixture was filtered and the filtrate was concentrated under reduced pressure to afford 2-(bromomethyl)-5-(2H-tetrazol-2-yl)pyridine, which was used in the next step without purification. LC/MS (m/z): 240, 242 (M+H)⁺

### Intermediate 155

### Preparation of (3-(pyridin-2-yl)bicyclo[1.1.1]pentan-1-yl)methyl methanesulfonate

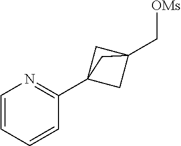 [SMILES: CS(=O)(=O)OCC12CC(c3ccccn3)(C1)C2]

### Step A: methyl 3-((tert-butylperoxy)carbonyl)bicyclo[1.1.1]pentane-1-carboxylate

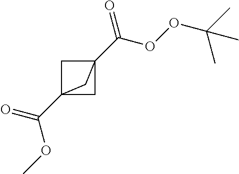 [SMILES: COC(=O)C12CC(C(=O)OOC(C)(C)C)(C1)C2]

A mixture of 3-(methoxycarbonyl)bicyclo[1.1.1]pentane-1-carboxylic acid (0.500 g, 2.94 mmol) in DCM (10 mL) was cooled to 0° C. and stirred under a nitrogen atmosphere for 5 minutes. EDCI HCl (0.620 g, 3.23 mmol), DMAP (36 mg, 0.29 mmol), and 2-hydroperoxy-2-methylpropane (0.65 mL, 3.2 mmol) were added to the reaction mixture and stirred at 0° C. under a nitrogen atmosphere for 10 minutes. The reaction mixture was warmed to room temperature and stirred for an additional 6 hours. The reaction mixture was washed with water (20 mL) and extracted with DCM (3×20 mL). The organic layers were combined, washed with saturated aqueous sodium bisulfite, dried over anhydrous sodium sulfate, filtered, and concentrated under reduced pressure. The residue was purified by silica gel chromatography (eluting ethyl acetate in petroleum ether) to afford methyl 3-((tert-butylperoxy)carbonyl)bicyclo[1.1.1]pentane-1-carboxylate. ¹H NMR (400 MHz, chloroform-d) δ 3.68 (s, 3H), 2.36 (s, 6H), 1.30 (s, 9H).

### Step B: methyl 3-(pyridin-2-yl)bicyclo[1.1.1]pentane-1-carboxylate

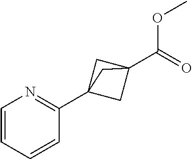

A mixture of methyl 3-((tert-butylperoxy)carbonyl)bicyclo[1.1.1]pentane-1-carboxylate (0.100 g, 0.413 mmol) and 02 in pyridine (2 mL) was stirred at 115° C. for 3 hours in a microwave reactor. The reaction mixture was concentrated under reduced pressure. The residue was purified by reverse phase HPLC (eluting acetonitrile in water, with TFA modifier) to afford methyl 3-(pyridin-2-yl)bicyclo[1.1.1]pentane-1-carboxylate. LC/MS (m/z): 204 (M+H)⁺

### Step C: (3-(pyridin-2-yl)bicyclo[1.1.1]pentan-1-yl)methanol

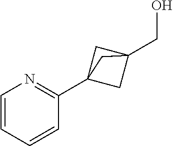 [SMILES: OCC12CC(c3ccccn3)(C1)C2]

LiBH₄ (16 mg, 0.75 mmol) was added to a mixture of methyl 3-(pyridin-2-yl)bicyclo[1.1.1]pentane-1-carboxylate (92 mg, 0.15 mmol) in THE (5 mL) at room temperature. The mixture was stirred for 2 hours at room temperature. The mixture was quenched with 1N HCl (1 mL). The mixture was filtered and concentrated under reduced pressure. The residue was purified by reverse phase HPLC (eluting acetonitrile in water, with TFA modifier) to afford (3-(pyridin-2-yl)bicyclo[1.1.1]pentan-1-yl)methanol. LC/MS (m/z): 176 (M+H)⁺

### Step D: (3-(pyridin-2-yl)bicyclo[1.1.1]pentan-1-yl)methanol

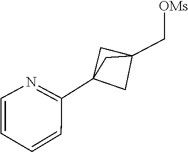 [SMILES: CS(=O)(=O)OCC12CC(c3ccccn3)(C1)C2]

Methanesulfonyl chloride (0.054 mL, 0.70 mmol) was added to a mixture of (3-(pyridin-2-yl)bicyclo[1.1.1]pentan-1-yl)methanol (35 mg, 0.067 mmol) and DIEA (0.035 mL, 0.20 mmol) in DCM (3 mL) at 0° C. The mixture was stirred at 0° C. for 2 hours. The mixture was quenched with water (10 mL) and extracted with DCM (15 mL). The organic layer was dried over anhydrous sodium sulfate, filtered, and concentrated under reduced pressure to afford (3-(pyridin-2-yl)bicyclo[1.1.1]pentan-1-yl)methyl methanesulfonate, which was used without purification in the next step. LC/MS (m/z): 254 (M+H)⁺

### Intermediate 156

### Preparation of 1-cyclobutyl-6-methylpiperazine-2,3-dione

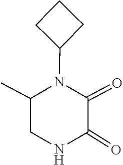

### Step A: tert-butyl (2-(cyclobutylamino)propyl)carbamate

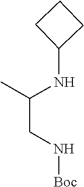 [SMILES: CC(CNC(=O)OC(C)(C)C)NC1CCC1]

Sodium cyanoborohydride (4.63 g, 73.6 mmol) was added to a mixture of cyclobutanamine (3.56 g, 50.1 mmol) and tert-butyl (2-oxopropyl)carbamate (8.5 g, 49 mmol) in MeOH (70 mL). The mixture was stirred at room temperature for 2 hours. The reaction mixture was diluted with saturated aqueous NaHCO₃ (20 mL) and extracted with DCM (3×50 mL). The organic layers were combined, dried over anhydrous sodium sulfate, filtered, and concentrated under reduced pressure. The residue was purified by silica gel chromatography (eluting methanol in DCM) to afford tert-butyl (2-(cyclobutylamino)propyl)carbamate. LC/MS (m/z): 229 (M+H)⁺

### Step B: ethyl 2-((1-((tert-butoxycarbonyl)amino)propan-2-yl)(cyclobutyl)amino)-2-oxoacetate

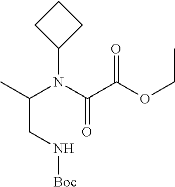 [SMILES: CCOC(=O)C(=O)N(C(C)CNC(=O)OC(C)(C)C)C1CCC1]

Ethyl 2-chloro-2-oxoacetate (4.58 mL, 40.9 mmol) was added to a mixture of tert-butyl (2-(cyclobutylamino)propyl)carbamate (8.5 g, 37 mmol) and TEA (7.8 ml, 56 mmol) in DCM (70 ml) at 0° C. The mixture was stirred at room temperature for 2 hours. The mixture was concentrated under reduced pressure and the residue was purified by silica gel chromatography (eluting ethyl acetate in petroleum ether) to afford ethyl 2-((1-((tert-butoxycarbonyl)amino)propan-2-yl)(cyclobutyl)amino)-2-oxoacetate. LC/MS (m/z): 229.1 [M+H-Boc]*.

### Step C: ethyl 2-((1-aminopropan-2-yl)(cyclobutyl)amino)-2-oxoacetate

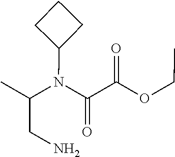 [SMILES: CCOC(=O)C(=O)N(C(C)CN)C1CCC1]

A mixture of ethyl 2-((1-((tert-butoxycarbonyl)amino)propan-2-yl)(cyclobutyl)amino)-2-oxoacetate (7.7 g, 23 mmol) and HCl (4.0 M in dioxane, 15 mL, 60 mmol) in DCM (70 mL) was stirred at room temperature for 2 hours. The mixture was concentrated under reduced pressure to afford ethyl 2-((1-aminopropan-2-yl)(cyclobutyl)amino)-2-oxoacetate hydrochloride, which was used in next step without purification. LC/MS (m/z): 229 (M+H)⁺

### Step D: (R or S)-1-cyclobutyl-6-methylpiperazine-2,3-dione and (S or R)-1-cyclobutyl-6-methylpiperazine-2,3-dione

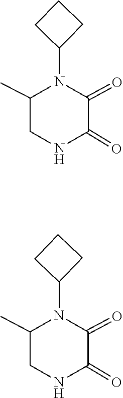

A mixture of ethyl 2-((1-aminopropan-2-yl)(cyclobutyl)amino)-2-oxoacetate hydrochloride (8.4 g, 32 mmol) and TEA (8.8 mL, 64 mmol) in EtOH (50 mL) was stirred and heated at 85° C. for 3 hours. The mixture was concentrated under reduced pressure and the residue was purified by silica gel chromatography (eluting MeOH in DCM) to afford 1-cyclobutyl-6-methylpiperazine-2,3-dione as a mixture of isomers. The mixture was resolved by Chiral-SFC (Column Daicel Chiralpak AS (250 mm×50 mm, 10 μm): eluting 40% EtOH in C₀₂ with 0.1% ammonia modifier) to afford (R or S)-1-cyclobutyl-6-methylpiperazine-2,3-dione (tᵣ=3.87 min) as the first eluting peak and (S or R)-1-cyclobutyl-6-methylpiperazine-2,3-dione (tᵣ=4.55 min) as the second eluting peak.

Peak 1: (R or S)-1-cyclobutyl-6-methylpiperazine-2,3-dione: ¹H NMR (400 MHz, chloroform-d) δ 8.08 (br s, 1H), 4.82-4.63 (m, 1H), 3.80 (br d, J=5.6 Hz, 1H), 3.73 (dd, J=3.7, 13.0 Hz, 1H), 3.27 (br dd, J=5.0, 12.8 Hz, 1H), 2.34-2.17 (m, 2H), 2.17-2.03 (m, 2H), 1.77-1.63 (m, 2H), 1.35 (d, J=6.6 Hz, 3H). LC/MS (m/z): 183 (M+H)⁺

Peak 2: (S or R)-1-cyclobutyl-6-methylpiperazine-2,3-dione: ¹H NMR (400 MHz, chloroform-d) δ 7.04-6.77 (m, 1H), 4.81-4.64 (m, 1H), 3.89-3.70 (m, 2H), 3.23 (dd, J=5.1, 12.5 Hz, 1H), 2.37-2.19 (m, 2H), 2.16-1.99 (m, 2H), 1.80-1.65 (m, 2H), 1.37 (d, J=6.4 Hz, 3H).

LC/MS (m/z): 183 (M+H)⁺

### Intermediate 157

### Preparation of 1-((6-phenylpyridazin-3-yl)methyl)piperazine-2,3-dione

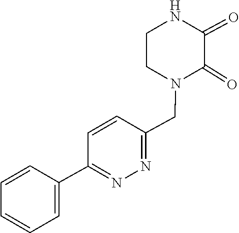 [SMILES: O=C1NCCN(Cc2ccc(-c3ccccc3)nn2)C1=O]

### Step A: tert-butyl (2-(((6-phenylpyridazin-3-yl)methyl)amino)ethyl)carbamate

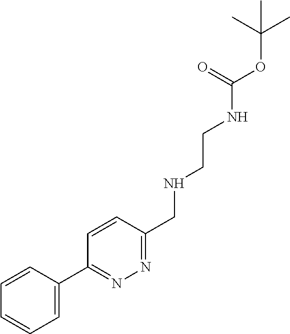 [SMILES: CC(C)(C)OC(=O)NCCNCc1ccc(-c2ccccc2)nn1]

3-(chloromethyl)-6-phenylpyridazine (0.550 g, 2.69 mmol) was added to a mixture of tert-butyl (2-aminoethyl)carbamate (1.29 g, 8.06 mmol) and potassium carbonate (743 mg, 5.37 mmol) in DMF (10 mL) at room temperature. The mixture was stirred and heated at 50° C. for 2 hours. The mixture was cooled to room temperature and filtered. The filtrate was concentrated under reduced pressure and the residue was purified by silica gel chromatography (eluting methanol in dichloromethane) to afford tert-butyl (2-(((6-phenylpyridazin-3-yl)methyl)amino)ethyl)carbamate. LC/MS (m/z): 329 (M+H)⁺

### Step B: ethyl 2-((2-((tert-butoxycarbonyl)amino)ethyl)((6-phenylpyridazin-3-yl)methyl)amino)-2-oxoacetate

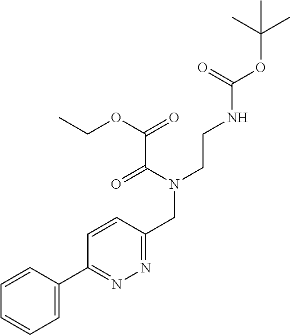 [SMILES: CCOC(=O)C(=O)N(CCNC(=O)OC(C)(C)C)Cc1ccc(-c2ccccc2)nn1]

Ethyl 2-chloro-2-oxoacetate (0.29 mL, 2.6 mmol) was added to a mixture of tert-butyl (2-(((6-phenylpyridazin-3-yl)methyl)amino)ethyl)carbamate (0.700 g, 2.13 mmol) and TEA (0.45 mL, 3.2 mmol) in DCM (10 mL) at 0° C. The mixture was stirred room temperature for 2 hours. The mixture was directly purified by silica gel chromatography (eluting ethyl acetate in petroleum ether) to afford ethyl 2-((2-((tert-butoxycarbonyl)amino)ethyl)((6-phenylpyridazin-3-yl)methyl)amino)-2-oxoacetate. ¹H NMR (400 MHz, DMSO-d₆) δ 8.24 (d, J=9.0 Hz, 1H), 7.63 (d, J=8.6 Hz, 1H), 7.61-7.41 (m, 5H), 6.92 (br s, 1H), 4.86 (s, 2H), 4.31 (q, J=7.0 Hz, 2H), 3.44 (br t, J=5.5 Hz, 2H), 3.19 (q, J=6.1 Hz, 2H), 1.37 (s, 9H), 1.30 (t, J=7.0 Hz, 3H). LC/MS (m/z): 429 (M+H)⁺

### Step C: ethyl 2-((2-aminoethyl)((6-phenylpyridazin-3-yl)methyl)amino)-2-oxoacetate

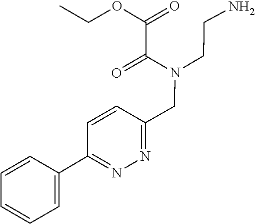 [SMILES: CCOC(=O)C(=O)N(CCN)Cc1ccc(-c2ccccc2)nn1]

A mixture of ethyl 2-((2-((tert-butoxycarbonyl)amino)ethyl)((6-phenylpyridazin-3-yl)methyl)amino)-2-oxoacetate (1.00 g, 2.33 mmol) and TFA (4.0 mL, 52 mmol) in DCM (10 mL) was stirred at room temperature for 2 hours. The mixture was concentrated under reduced pressure to afford ethyl 2-((2-aminoethyl)((6-phenylpyridazin-3-yl)methyl)amino)-2-oxoacetate, which was used in the next step without purification. LC/MS (m/z): 329 (M+H)⁺

### Step D: 1-((6-phenylpyridazin-3-yl)methyl)piperazine-2,3-dione

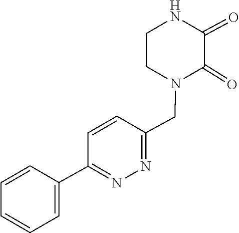 [SMILES: O=C1NCCN(Cc2ccc(-c3ccccc3)nn2)C1=O]

TEA (0.891 mL, 6.40 mmol) was added to a mixture of ethyl 2-((2-aminoethyl)((6-phenylpyridazin-3-yl)methyl)amino)-2-oxoacetate (0.700 g, 2.13 mmol) in EtOH (15 mL) at room temperature. The mixture was stirred and heated at 80° C. for 12 hours. The mixture was concentrated under reduced pressure. The residue was diluted with MTBE and stirred at room temperature for 10 minutes. The mixture was filtered and the filtrate was concentrated under reduced pressure to afford -((6-phenylpyridazin-3-yl)methyl)piperazine-2,3-dione, which was used without purification in the next step. ¹H NMR (500 MHz, DMSO-d₆) δ 8.67 (br s, 1H), 8.22 (d, J=8.9 Hz, 1H), 8.17-8.12 (m, 2H), 7.74 (d, J=8.9 Hz, 1H), 7.60-7.52 (m, 3H), 4.92 (s, 2H), 3.70-3.63 (m, 2H), 3.44-3.38 (m, 2H). LC/MS (m/z): 283 (M+H)⁺

Examples shown in Intermediate Table 18 below, were prepared according to procedures analogous to those outlined in Intermediate 157 above using the appropriate starting materials, described in the Preparations or Intermediates above, or as obtained from commercial sources.

Intermediate Table 18

| Intermediate | Structure | Name | Mass [M + H]⁺ |
|---|---|---|---|
| Intermediate 158 | 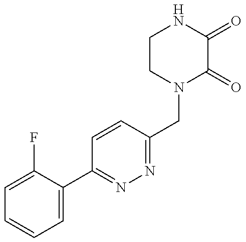 [SMILES: O=C1NCCN(Cc2ccc(-c3ccccc3F)nn2)C1=O] | 1-((6-(2- fluorophenyl)pyridazin-3- yl)methyl)piperazine-2,3- dione | 301 |

### Intermediate 159

### Preparation of hexahydro-2H-pyrido[1,2-a]pyrazine-3,4-dione

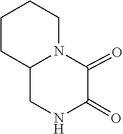 [SMILES: O=C1NCC2CCCCN2C1=O]

A mixture of piperidin-2-ylmethanamine (2.00 g, 17.5 mmol), diethyl oxalate (5.12 g, 35.0 mmol), and TEA (3.66 mL, 26.3 mmol) in ethanol (25 mL) was stirred and heated at 90° C. for 2 hours. The mixture was concentrated under reduced pressure and the residue was purified by silica gel chromatography (eluting MeOH in DCM) to afford hexahydro-2H-pyrido[1,2-a]pyrazine-3,4-dione. ¹H NMR (400 MHz, DMSO-d₆) δ 8.50 (br s, 1H), 4.29-4.21 (m, 1H), 3.61-3.51 (m, 1H), 3.38 (td, J=4.34, 12.90 Hz, 1H), 3.19-3.12 (m, 1H), 2.65 (dt, J=3.16, 12.90 Hz, 1H), 1.80-1.68 (m, 3H), 1.39-1.24 (m, 3H). LC/MS (m/z): 169 (M+H)⁺

### Intermediate 160

### Preparation of 5-(chloromethyl)-3-phenylisoxazole

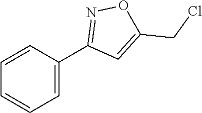 [SMILES: ClCc1cc(-c2ccccc2)no1]

Thionyl chloride (0.167 mL, 2.28 mmol) was added to a mixture of (3-phenylisoxazol-5-yl)methanol (0.200 g, 1.14 mmol) in DCM (5 mL) at 0° C. The mixture was stirred at room temperature for 2 hours. The reaction mixture was concentrated under reduced pressure to afford 5-(chloromethyl)-3-phenylisoxazole, which was used without purification in the next step. LC/MS (m/z): 194 (M+H)⁺

### Intermediate 161

### Preparation of 1-(bicyclo[2.1.1]hexan-2-yl)piperazine-2,3-dione

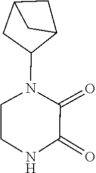 [SMILES: O=C1NCCN(C2CC3CC2C3)C1=O]

### Step A: tert-butyl (2-(bicyclo[2.1.1]hexan-2-ylamino)ethyl)carbamate

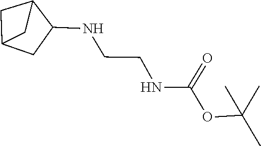 [SMILES: CC(C)(C)OC(=O)NCCNC1CC2CC1C2]

A mixture of bicyclo[2.1.1]hexan-2-amine HCl (191 mg, 1.43 mmol), tert-butyl (2-bromoethyl)carbamate (0.320 g, 1.43 mmol), and potassium phosphate tribasic (606 mg, 2.86 mmol) in DMF (7.1 mL) was stirred and heated to 60° C. for 24 hours. The mixture was filtered, and the filtrate was concentrated under reduced pressure to afford tert-butyl (2-(bicyclo[2.1.1]hexan-2-ylamino)ethyl)carbamate, which was used without purification in the next step. LC/MS (m/z): 241 (M+H)⁺

### Step B: N1-(bicyclo[2.1.1]hexan-2-yl)ethane-1,2-diamine hydrochloride

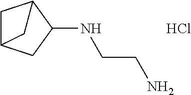

A mixture of tert-butyl (2-(bicyclo[2.1.1]hexan-2-ylamino)ethyl)carbamate (343 mg, 1.43 mmol), HCl (4.0 M in dioxane, 5.3 mL, 21 mmol) in dioxane (7 mL) was stirred and heated to 50° C. for 1 hour. The mixture was cooled to room temperature and concentrated under reduced pressure to afford N1-(bicyclo[2.1.1]hexan-2-yl)ethane-1,2-diamine hydrochloride, which was used without purification in the next step. LC/MS (m/z): 141 (M+H)⁺

### Step C: 1-(bicyclo[2.1.1]hexan-2-yl)piperazine-2,3-dione

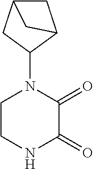 [SMILES: O=C1NCCN(C2CC3CC2C3)C1=O]

A solution of diethyl oxalate (461 μL, 3.40 mmol) in EtOH (5.6 mL) was added to a mixture of N1-(bicyclo[2.1.1]hexan-2-yl)ethane-1,2-diamine hydrochloride (0.200 g, 1.13 mmol) and TEA (316 μl, 2.26 mmol) at room temperature. The mixture was stirred and heated at 80° C. for 18 hours. The mixture was cooled to room temperature and concentrated under reduced pressure. The residue was purified by silica gel chromatography (eluting ethyl acetate in hexanes) to afford 1-(bicyclo[2.1.1]hexan-2-yl)piperazine-2,3-dione. LC/MS (m/z): 195 (M+H)⁺

### Intermediate 162

### Preparation of (5-(thiazol-2-yl)isoxazol-3-yl)methyl methanesulfonate

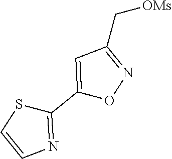 [SMILES: CS(=O)(=O)OCc1cc(-c2nccs2)on1]

### Step A: ethyl 5-(thiazol-2-yl)isoxazole-3-carboxylate

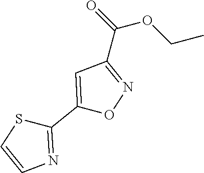

A mixture of ethyl 5-bromoisoxazole-3-carboxylate (360 mg, 1.64 mmol), Pd(Ph₃P)₄ (189 mg, 0.164 mmol), and 2-(tributylstannyl)thiazole (0.617 mL, 1.963 mmol) in toluene (6 mL) was degassed and backfilled with nitrogen (3×). The mixture was stirred and heated at 100° C. for 16 hours. The mixture was quenched with saturated aqueous KF (3 mL) and extracted with EtOAc (3×10 mL). The organic layers were combined, washed with brine (3 mL), dried over anhydrous sodium sulfate, filtered, and concentrated under reduced pressure. The residue was purified by silica gel chromatography (eluting ethyl acetate in petroleum ether) to afford ethyl 5-(thiazol-2-yl)isoxazole-3-carboxylate. ¹H NMR (400 MHz, DMSO-d₆) δ 8.17-8.11 (m, 2H), 7.50 (s, 1H), 4.41 (q, J=7.11 Hz, 2H), 1.37-1.33 (m, 3H). LC/MS (m/z): 225 (M+H)⁺

### Step B: (5-(thiazol-2-yl)isoxazol-3-yl)methanol

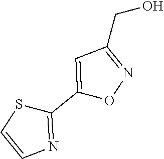 [SMILES: OCc1cc(-c2nccs2)on1]

Sodium borohydride (68 mg, 1.8 mmol) was added to a mixture of ethyl 5-(thiazol-2-yl)isoxazole-3-carboxylate (0.200 g, 0.892 mmol) in MeOH (5 mL) at 0° C. The mixture was stirred at room temperature for 2 hours. The reaction mixture was quenched with water (3 mL) and extracted with EtOAc (3×8 mL). The organic layers were combined, washed with brine (2 mL), dried over anhydrous sodium sulfate, filtered, and concentrated under reduced pressure. The residue was purified by silica gel chromatography (eluting ethyl acetate in petroleum ether) to afford (5-(thiazol-2-yl)isoxazol-3-yl)methanol. LC/MS (m/z): 183 (M+H)⁺

### Step C: (5-(thiazol-2-yl)isoxazol-3-yl)methyl methanesulfonate

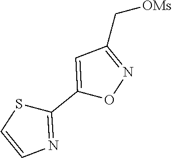 [SMILES: CS(=O)(=O)OCc1cc(-c2nccs2)on1]

Methanesulfonic anhydride (93 mg, 0.54 mmol) was added to a mixture of (5-(thiazol-2-yl)isoxazol-3-yl)methanol (65 mg, 0.36 mmol) and TEA (0.099 ml, 0.71 mmol) in DCM (4 mL). The mixture was stirred at room temperature for 2 hours. The mixture was concentrated under reduced pressure to afford (5-(thiazol-2-yl)isoxazol-3-yl)methyl methanesulfonate, which was used in the next step without purification. LC/MS (m/z): 261 (M+H)⁺

### Intermediate 163

### Preparation of (2,2-difluoro-3-phenylbicyclo[1.1.1]pentan-1-yl)methyl methanesulfonate

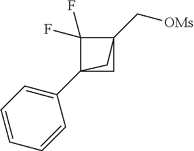 [SMILES: CS(=O)(=O)OCC12CC(c3ccccc3)(C1)C2(F)F]

### Step A: (2,2-difluoro-3-phenylbicyclo[1.1.1]pentan-1-yl)methanol

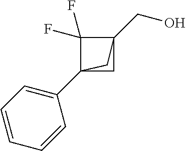 [SMILES: OCC12CC(c3ccccc3)(C1)C2(F)F]

LAH (1.0 M in THF, 669 μl, 0.67 mmol) was added dropwise to a mixture of 2,2-difluoro-3-phenylbicyclo[1.1.1]pentane-1-carboxylic acid (50 mg, 0.22 mmol) in THE (2.2 mL) at room temperature. The mixture was stirred and heated to 60° C. for 2 hours. The mixture was cooled to 0° C. and quenched dropwise with saturated aqueous ammonium chloride. The mixture was washed with DCM (3×5 mL) and the organic layers were combined, dried over sodium sulfate, filtered, and concentrated under reduced pressure to afford (2,2-difluoro-3-phenylbicyclo[1.1.1]pentan-1-yl)methanol, which was used without purification in the next step. ¹H NMR (499 MHz, chloroform-d) δ 7.40-7.30 (m, 5H), 3.95 (s, 2H), 3.80-3.75 (m, 1H), 2.38 (s, 2H), 1.90-1.86 (m, 1H), 1.82 (t, J=10.7 Hz, 2H).

### Step B: (2,2-difluoro-3-phenylbicyclo[1.1.1]pentan-1-yl)methyl methanesulfonate

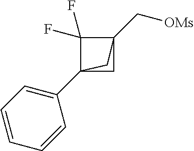 [SMILES: CS(=O)(=O)OCC12CC(c3ccccc3)(C1)C2(F)F]

Methanesulfonyl chloride (40 μl, 0.52 mmol) was added to a mixture of (2,2-difluoro-3-phenylbicyclo[1.1.1]pentan-1-yl)methanol (0.050 g, 0.24 mmol) and triethylamine (80 μl, 0.57 mmol) in DCM (2.4 mL) at 0° C. The mixture was stirred at 0° C. for 2 hours. The mixture was quenched with water (10 mL) and extracted with DCM (15 mL). The organic layer was separated, dried over Na₂SO₄, filtered, and concentrated under reduced pressure to afford (2,2-difluoro-3-phenylbicyclo[1.1.1]pentan-1-yl)methyl methanesulfonate, which was used in the next step without purification. LC/MS (m/z): 311 (M+Na)⁺

### Intermediate 164

### Preparation of 2-azidopyridine

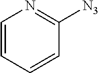

NaN₃ (1.42 g, 21.8 mmol) and ammonium chloride (0.942 g, 17.6 mmol) were added to a mixture of 2-chloropyridine (1.00 g, 8.81 mmol) in DMF (15 mL) at room temperature over a period of 5 minutes. The mixture was stirred and heated to 110° C. for 16 hours. The mixture was adjusted to pH >9 by the addition of aqueous sodium bicarbonate and then extracted with EtOAc (5×40 mL). The organic layers were combined, washed with brine (50 mL), dried over anhydrous sodium sulfate, filtered, and concentrated under reduced pressure to afford 2-azidopyridine, which was used in next step without purification. ¹H NMR (500 MHz, methanol-d₄) δ 9.14 (td, J=1.03, 6.94 Hz, 1H), 8.12 (td, J=0.99, 9.00 Hz, 1H), 8.00 (s, 1H), 7.90-7.86 (m, 1H).

### Intermediate 165

### Preparation of 3-azidopyridine

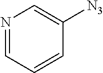

TBAF (1.0 M in THF, 19.5 mL, 20 mmol), TMS-N₃ (2.6 mL, 20 mmol), and CuCi (0.161 g, 1.63 mmol) were added to a mixture of pyridin-3-ylboronic acid (2.00 g, 16.3 mmol) in MeOH (40 mL) at room temperature. The mixture was stirred and heated at 80° C. for 16 hours. The mixture was concentrated under reduced pressure and the residue was partitioned between water (20 mL) and EtOAc (20 mL). The organic layer was separated and the aqueous was re-extracted with EtOAc (3×15 mL). The organic layers were combined, washed with brine (20 mL), dried over anhydrous sodium sulfate, filtered, and concentrated under reduced pressure. The residue was purified by silica gel chromatography (eluting ethyl acetate in petroleum ether) to afford 3-azidopyridine. ¹H NMR (400 MHz, methanol-d₄) δ 8.23-8.14 (m, 2H), 7.51-7.44 (m, 1H), 7.33 (dd, J=4.8, 8.4 Hz, 1H).

### Intermediate 166

### Preparation of 2-(6-(chloromethyl)pyridazin-3-yl)thiazole

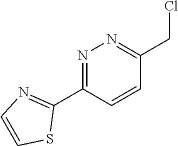 [SMILES: ClCc1ccc(-c2nccs2)nn1]

### Step A: 2-(6-methylpyridazin-3-yl)thiazole

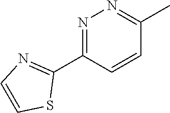

A mixture of 3-bromo-6-methylpyridazine (0.500 g, 2.89 mmol), 2-(tributylstannyl)thiazole (1.82 mL, 5.78 mmol), and Pd(Ph₃P)₄ (334 mg, 0.289 mmol) in toluene (15 mL) was degassed and backfilled with N₂ (3×). The mixture was stirred and heated to 100° C. for 16 hours. The mixture was filtered and the filtrate was concentrated under reduced pressure. The residue was purified by silica gel chromatography (eluting ethyl acetate in petroleum ether) to afford 2-(6-methylpyridazin-3-yl)thiazole. LC/MS (m/z): 178 (M+H)⁺

### Step B: 2-(6-(chloromethyl)pyridazin-3-yl)thiazole

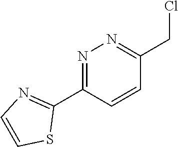 [SMILES: ClCc1ccc(-c2nccs2)nn1]

A mixture of 2-(6-methylpyridazin-3-yl)thiazole (680 mg, 1.92 mmol) and trichloroisocyanuric acid (178 mg, 0.767 mmol) in CHCl₃ (18 mL) was stirred and heated at 60° C. for 3 hours. The mixture was filtered and the filtrate was purified by silica gel chromatography (eluting ethyl acetate in petroleum ether) to afford 2-(6-(chloromethyl)pyridazin-3-yl)thiazole. LC/MS (m/z): 212 (M+H)⁺

### Intermediate 167

### Preparation of 2-(chloromethyl)-5-(1H-pyrazol-1-yl)pyridine

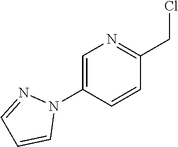 [SMILES: ClCc1ccc(-n2cccn2)cn1]

### Step A: (5-(1H-pyrazol-1-yl)pyridin-2-yl)methanol

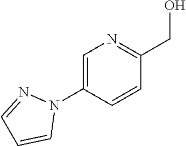 [SMILES: OCc1ccc(-n2cccn2)cn1]

A mixture of (5-bromopyridin-2-yl)methanol (1.50 g, 7.98 mmol), 1H-pyrazole (0.815 g, 12.0 mmol), copper(I) iodide (0.152 g, 0.798 mmol), N1,N2-dimethylethane-1,2-diamine (0.105 g, 1.20 mmol), and potassium carbonate (1.65 g, 12.0 mmol) in DMF (20 mL) was stirred and heated at 100° C. for 16 hours. The mixture was quenched with water (20 mL) and extracted with EtOAc (3×20 mL). The organic layers were combined, washed with brine (20 mL), dried over anhydrous sodium sulfate, filtered, and concentrated under reduced pressure. The residue was purified by silica gel chromatography (eluting ethyl acetate in petroleum ether) to afford (5-(1H-pyrazol-1-yl)pyridin-2-yl)methanol. LC/MS (m/z): 176 (M+H)⁺

### Step B: 2-(chloromethyl)-5-(1H-pyrazol-1-yl)pyridine

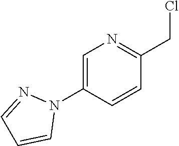 [SMILES: ClCc1ccc(-n2cccn2)cn1]

Thionyl chloride (0.146 mL, 2.00 mmol) was added to a mixture of (5-(1H-pyrazol-1-yl)pyridin-2-yl)methanol (0.070 g, 0.40 mmol) in DCM (1 mL) at 0° C. The mixture was stirred for 2.5 hours at 0° C. The mixture was concentrated under reduced pressure to afford 2-(chloromethyl)-5-(1H-pyrazol-1-yl)pyridine, which was used in the next step without purification. LC/MS (m/z): 194 (M+H)⁺

Examples shown in Intermediate Table 19 below, were prepared according to procedures analogous to those outlined in Intermediate 167 above using the appropriate starting materials, described in the Preparations or Intermediates above, or as obtained from commercial sources.

Intermediate Table 19

| Intermediate | Structure | Name | Mass [M + H]⁺ |
|---|---|---|---|
| Intermediate 168 | 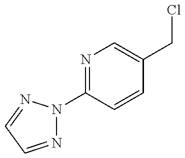 [SMILES: ClCc1ccc(-n2nccn2)nc1] | 5-(chloromethyl)-2-(2H-1,2,3- triazol-2-yl)pyridine | 195 |

### Intermediate 169

### Preparation of 5-(chloromethyl)-2-(1H-pyrazol-1-yl)pyridine

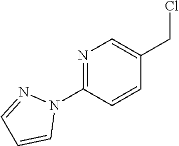 [SMILES: ClCc1ccc(-n2cccn2)nc1]

### Step A: (6-(1H-pyrazol-1-yl)pyridin-3-yl)methanol

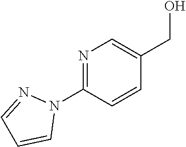 [SMILES: OCc1ccc(-n2cccn2)nc1]

A mixture of (6-chloropyridin-3-yl)methanol (0.300 g, 2.09 mmol), 1H-pyrazole (427 mg, 6.27 mmol), and cesium carbonate (2040 mg, 6.27 mmol) in NMP (5 mL) was stirred and heated at 150° C. for 1 hour. The mixture was quenched with water (150 mL) and extracted with EtOAc (3×200 mL). The organic layers were combined, washed with brine (20 mL), dried over anhydrous sodium sulfate, filtered, and concentrated under reduced pressure. The residue was purified by silica gel chromatography (eluting ethyl acetate in petroleum ether) to afford (6-(1H-pyrazol-1-yl)pyridin-3-yl)methanol. ¹H NMR (400 MHz, chloroform-d) δ 8.57 (d, J=2.4 Hz, 1H), 8.40 (d, J=1.6 Hz, 1H), 7.99 (d, J=8.4 Hz, 1H), 7.86 (dd, J=2.2, 8.4 Hz, 1H), 7.75 (s, 1H), 6.52-6.44 (m, 1H), 4.77 (s, 2H). LC/MS (m/z): 176 (M+H)⁺

### Step B: 5-(chloromethyl)-2-(1H-pyrazol-1-yl)pyridine

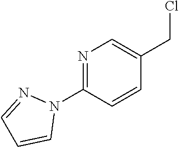 [SMILES: ClCc1ccc(-n2cccn2)nc1]

Thionyl chloride (0.052 mL, 0.71 mmol) was added to a mixture of (6-(1H-pyrazol-1-yl)pyridin-3-yl)methanol (0.050 g, 0.29 mmol) in DCM (1 mL) 0° C. The mixture was stirred at room temperature for 1 hour. The mixture was concentrated under reduced pressure to afford 5-(chloromethyl)-2-(1H-pyrazol-1-yl)pyridine, which was used in next step without purification. LC/MS (m/z): 194 (M+H)⁺

Examples shown in Intermediate Table 20 below, were prepared according to procedures analogous to those outlined in Intermediate 169 above using the appropriate starting materials, described in the Preparations or Intermediates above, or as obtained from commercial sources.

Intermediate Table 20

| Intermediate | Structure | Name | Mass [M + H]⁺ |
|---|---|---|---|
| Intermediate 170 | 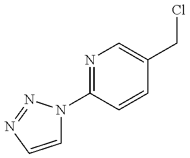 [SMILES: ClCc1ccc(-n2ccnn2)nc1] | 5-(chloromethyl)-2-(1H-1,2,3- triazol-1-yl)pyridine | 195 |

### Intermediate 171

### Preparation of (3-(pyridin-3-yl)isoxazol-5-yl)methyl methanesulfonate

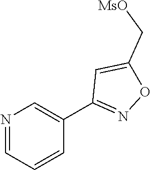 [SMILES: CS(=O)(=O)OCc1cc(-c2cccnc2)no1]

### Step A: nicotinaldehyde oxime

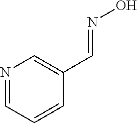

Hydroxylamine hydrochloride (7.78 g, 112 mmol) was added to a mixture of nicotinaldehyde (8.8 mL, 93 mmol) in EtOH (200 mL) at room temperature. After stirring for 5 minutes at room temperature, sodium acetate (15.3 g, 187 mmol) was added to the mixture. The mixture was stirred for another 2 hours at room temperature. The mixture was cooled, diluted with water (100 mL), and extracted with ethyl acetate (200 mL). The organic layer was washed with brine (100 mL), dried over sodium sulfate, filtered, and concentrated under reduced pressure to afford nicotinaldehyde oxime, which was used without purification in the next step. ¹H NMR (methanol-d₄, 500 MHz) δ 8.71 (d, J=1.5 Hz, 1H), 8.50-8.49 (m, 1H), 8.20-8.10 (m, 1H), 8.14-8.06 (m, 1H), 7.45-7.42 (m, 1H).

### Step B: N-hydroxynicotinimidoyl chloride

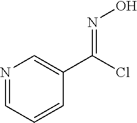

NCS (12.7 g, 95.0 mmol) was added to a mixture of nicotinaldehyde oxime (10.8 g, 88.0 mmol) and pyridine (0.715 mL, 8.84 mmol) in THE (150 mL) at room temperature. The mixture was stirred and heated at 50° C. for 1 hour. The mixture was quenched with water (50 mL) and extracted with EtOAc (3×100 mL). The organic layers were combined, washed with brine (50 mL), dried over anhydrous sodium sulfate, filtered, and concentrated under reduced pressure. The residue was purified by silica gel chromatography (eluting ethyl acetate in petroleum ether) to afford N-hydroxynicotinimidoyl chloride. ¹H NMR (400 MHz, DMSO-d₆) δ 12.70 (s, 1H), 8.96 (d, J=1.9 Hz, 1H), 8.67 (dd, J=1.3, 4.8 Hz, 1H), 8.14 (td, J=1.9, 8.0 Hz, 1H), 7.53 (dd, J=4.8, 8.0 Hz, 1H). LC/MS (m/z): 157 (M+H)⁺

### Step C: (3-(pyridin-3-yl)isoxazol-5-yl)methanol

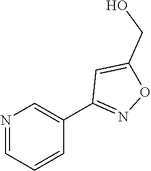 [SMILES: OCc1cc(-c2cccnc2)no1]

Prop-2-yn-1-ol (4.9 mL, 82 mmol) and TEA (5.34 mL, 38.3 mmol) were added to a mixture of N-hydroxynicotinimidoyl chloride (5.00 g, 31.9 mmol) in THE (50 mL) at room temperature under a nitrogen atmosphere. The mixture was stirred and heated at 50° C. for 16 hours. The mixture was quenched with saturated aqueous NaHCO₃ (35 mL) and extracted with EtOAc (3×50 mL). The organic layers were combined, dried over anhydrous sodium sulfate, filtered, and concentrated under reduced pressure. The residue was purified by silica gel chromatography (eluting neat ethyl acetate) to afford (3-(pyridin-3-yl)isoxazol-5-yl)methanol. ¹H NMR (400 MHz, DMSO-d₆) δ 9.08 (d, J=1.6 Hz, 1H), 8.69 (dd, J=1.6, 4.9 Hz, 1H), 8.26 (td, J=1.9, 8.0 Hz, 1H), 7.55 (dd, J=4.8, 7.9 Hz, 1H), 7.05 (s, 1H), 5.76 (t, J=6.0 Hz, 1H), 4.64 (d, J=6.0 Hz, 2H). LC/MS (m/z): 177 (M+H)⁺

### Step D: (3-(pyridin-3-yl)isoxazol-5-yl)methyl methanesulfonate

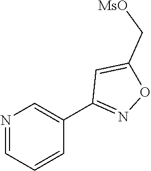 [SMILES: CS(=O)(=O)OCc1cc(-c2cccnc2)no1]

TEA (0.593 mL, 4.26 mmol) and methanesulfonic anhydride (742 mg, 4.26 mmol) were added to a mixture of (3-(pyridin-3-yl)isoxazol-5-yl)methanol (0.500 g, 2.84 mmol) in DCM (10 mL) at 0° C. The mixture was stirred at room temperature for 2 hours. The mixture was quenched with saturated aqueous NaHCO₃ (15 mL) and extracted with DCM (3×20 mL). The organic layers were combined, washed with brine (15 mL), dried over anhydrous sodium sulfate, filtered, and concentrated under reduced pressure to afford (3-(pyridin-3-yl)isoxazol-5-yl)methyl methanesulfonate, which was used without purification in the next step. LC/MS (m/z): 255 (M+H)⁺

Examples shown in Intermediate Table 21 below, were prepared according to procedures analogous to those outlined in Intermediate 171 above using the appropriate starting materials, described in the Preparations or Intermediates above, or as obtained from commercial sources.

Intermediate Table 21

| Intermediate | Structure | Name | Mass [M + H]⁺ |
|---|---|---|---|
| Intermediate 172 | 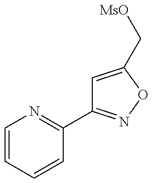 [SMILES: CS(=O)(=O)OCc1cc(-c2ccccn2)no1] | (3-(pyridin-2-yl)isoxazol-5- yl)methyl methanesulfonate | 255 |

### Intermediate 173

### Preparation of 1-(3-fluorobicyclo[1.1.1]pentan-1-yl)piperazine-2,3-dione

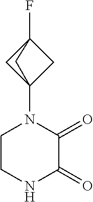 [SMILES: O=C1NCCN(C23CC(F)(C2)C3)C1=O]

Pd—C (10% w/w, 1627 mg, 1.5 mmol) was added to a mixture of 1-(3-fluorobicyclo[1.1.1]pentan-1-yl)-1,4-dihydropyrazine-2,3-dione (0.300 g, 1.53 mmol) in MeOH (5 mL) under a N₂ atmosphere. The mixture was degassed and backfilled with H₂ (three times). The mixture was stirred under a H₂ atmosphere (15 psi) and heated at 60° C. for 1 hour. The mixture was filtered and the filtrate was concentrated under reduced pressure to afford 1-(3-fluorobicyclo[1.1.1]pentan-1-yl)piperazine-2,3-dione, which was used without purification in the next step. LC/MS (m/z): 199 (M+H)⁺

### Intermediate 174

### Preparation of 6′-(chloromethyl)-2,3′-bipyridine

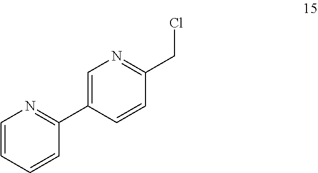 [SMILES: ClCc1ccc(-c2ccccn2)cn1]

### Step A: [2,3′-bipyridin]-6′-ylmethanol

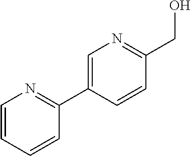 [SMILES: OCc1ccc(-c2ccccn2)cn1]

A mixture of (5-(4,4,5,5-tetramethyl-1,3,2-dioxaborolan-2-yl)pyridin-2-yl)methanol (2.50 g, 10.6 mmol), 2-bromopyridine (1.34 g, 8.51 mmol), and PdCl₂(dppf) (0.389 g, 0.532 mmol) in a mixture of Na₂CO₃ (2.0 M in water, 16 mL, 32 mmol) and dioxane (50 mL) was stirred and heated at 100° C. for 2 hours. The mixture was quenched with saturated aqueous NH₄Cl (20 mL) and extracted with EtOAc (3×30 mL). The organic layers were combined, washed with brine (50 mL), dried over anhydrous sodium sulfate, filtered, and concentrated under reduced pressure. The residue was purified by silica gel chromatography (eluting methanol in dichloromethane) to afford [2,3′-bipyridin]-6′-ylmethanol. LC/MS (m/z): 187 (M+H)⁺

### Step B: 6′-(chloromethyl)-2,3′-bipyridine

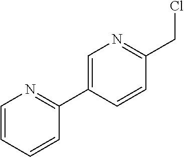 [SMILES: ClCc1ccc(-c2ccccn2)cn1]

Thionyl chloride (353 μL, 4.84 mmol) was added to a mixture of [2,3′-bipyridin]-6′-ylmethanol (0.200 g, 1.07 mmol) in DCM (10 mL) at 0° C. The mixture was warmed to room temperature and stirred for 2 hours. The mixture was concentrated under reduced pressure to afford 6′-(chloromethyl)-2,3′-bipyridine, which was used in the next step without purification. LC/MS (m/z): 205 (M+H)⁺

### Intermediate 175

### Preparation of 3-(chloromethyl)-6-(1H-1,2,3-triazol-1-yl)pyridazine

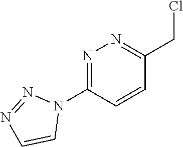 [SMILES: ClCc1ccc(-n2ccnn2)nn1]

### Step A: 3-methyl-6-(1H-1,2,3-triazol-1-yl)pyridazine

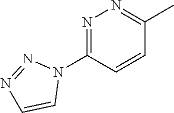

Cs₂CO₃ (3.80 g, 11.7 mmol) was added to a mixture of 3-chloro-6-methylpyridazine (1.00 g, 7.78 mmol) and 2H-1,2,3-triazole (1.40 mL, 23.3 mmol) in DMF (20 mL). The mixture was stirred and heated at 100° C. for 3 hours. The mixture was filtered and the filtrate was concentrated under reduced pressure. The residue was purified by silica gel chromatography (eluting ethyl acetate in petroleum ether) to afford 3-methyl-6-(2H-1,2,3-triazol-2-yl)pyridazine. LC/MS (m/z): 162 (M+H)⁺

### Step B: 3-(chloromethyl)-6-(1H-1,2,3-triazol-1-yl)pyridazine

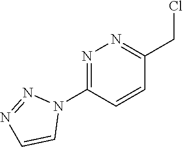 [SMILES: ClCc1ccc(-n2ccnn2)nn1]

A mixture of 3-methyl-6-(1H-1,2,3-triazol-1-yl)pyridazine (0.400 g, 2.48 mmol) and trichloroisocyanuric acid (288 mg, 1.24 mmol) in CHCl₃ (15 mL) was stirred and heated at 60° C. for 3 hours. The mixture was filtered and the filtrate was purified by silica gel chromatography (eluting ethyl acetate in petroleum ether) to afford 3-(chloromethyl)-6-(1H-1,2,3-triazol-1-yl)pyridazine. LC/MS (m/z): 196 (M+H)⁺

### Intermediate 176

### Preparation of 3-(chloromethyl)-5-(pyridin-2-yl)isoxazole

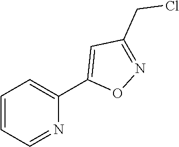 [SMILES: ClCc1cc(-c2ccccn2)on1]

Thionyl chloride (0.041 mL, 0.57 mmol) was added to a mixture of (5-(pyridin-2-yl)isoxazol-3-yl)methanol (0.050 g, 0.28 mmol) in DCM (2 mL) at 0° C. The mixture was stirred at room temperature for 1 hour. The mixture was concentrated under reduced pressure to afford 3-(chloromethyl)-5-(pyridin-2-yl)isoxazole, which was used in the next step without purification. LC/MS (m/z): 195 (M+H)⁺

### Intermediate 177

### Preparation of 3-(chloromethyl)-5-(pyridin-3-yl)isoxazole

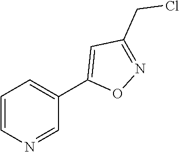 [SMILES: ClCc1cc(-c2cccnc2)on1]

### Step A: ethyl 5-(pyridin-3-yl)isoxazole-3-carboxylate

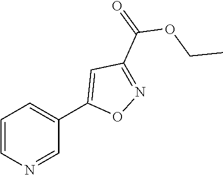 [SMILES: CCOC(=O)c1cc(-c2cccnc2)on1]

A mixture of 3-ethynylpyridine (2.00 g, 19.4 mmol), ethyl (E)-2-chloro-2-(hydroxyimino)acetate (8.82 g, 58.2 mmol), and NaHCO₃ (4.89 g, 58.2 mmol) in EtOAc (12 mL) was stirred and heated at 100° C. for 1 hour in a microwave. The mixture was filtered and the filtrate was purified by silica gel chromatography (eluting ethyl acetate in petroleum ether) to afford ethyl 5-(pyridin-3-yl)isoxazole-3-carboxylate. LC/MS (m/z): 219 (M+H)⁺

### Step B: (5-(pyridin-3-yl)isoxazol-3-yl)methanol

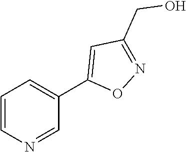 [SMILES: OCc1cc(-c2cccnc2)on1]

NaBH₄ (1.98 g, 52.2 mmol) was added to a mixture of ethyl 5-(pyridin-3-yl)isoxazole-3-carboxylate (3.80 g, 17.4 mmol) in MeOH (50 mL) at 0° C. The mixture was stirred and heated at 50° C. for 24 hours. The mixture was quenched with saturated aqueous NH₄Cl (100 mL) and extracted with EtOAc (3×100 mL). The organic layers were combined, washed with brine (20 mL), dried over anhydrous sodium sulfate, filtered, and concentrated under reduced pressure. The residue was purified by silica gel chromatography (eluting ethyl acetate in petroleum ether) to afford (5-(pyridin-3-yl)isoxazol-3-yl)methanol. ¹H NMR (400 MHz, DMSO-d₆) δ 9.10 (d, J=1.8 Hz, 1H), 8.68 (dd, J=1.5, 4.8 Hz, 1H), 8.26 (td, J=1.9, 8.0 Hz, 1H), 7.57 (dd, J=4.8, 7.9 Hz, 1H), 7.17 (s, 1H), 5.59 (t, J=5.9 Hz, 1H), 4.57 (d, J=5.8 Hz, 2H). LC/MS (m/z): 177 (M+H)⁺

### Step C: 3-(chloromethyl)-5-(pyridin-3-yl)isoxazole

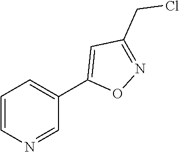 [SMILES: ClCc1cc(-c2cccnc2)on1]

Thionyl chloride (0.041 mL, 0.57 mmol) was added to a mixture of (5-(pyridin-2-yl)isoxazol-3-yl)methanol (0.050 g, 0.28 mmol) in DCM (2 mL) at 0° C. The mixture was stirred at room temperature for 1 hour. The mixture was concentrated under reduced pressure to afford 3-(chloromethyl)-5-(pyridin-2-yl)isoxazole, which was used in the next step without purification. LC/MS (m/z): 195 (M+H)⁺

### Intermediate 178

### Preparation of [2,3′-bipyridin]-6′-ylmethyl methanesulfonate

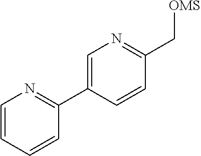

Methanesulfonic anhydride (103 mg, 0.591 mmol) was added to a mixture of [2,3′-bipyridin]-6′-ylmethanol (0.100 g, 0.537 mmol) and TEA (0.082 mL, 0.59 mmol) in DCM (4 mL) at 0° C. The mixture was stirred at room temperature for 1 hour. The mixture was quenched with water (3 mL) and extracted with DCM (3×3 mL). The organic layers were combined, washed with brine (2 mL), dried over anhydrous sodium sulfate, filtered, and concentrated under reduced pressure to afford [2,3′-bipyridin]-6′-ylmethyl methanesulfonate, which was used in the next step without purification. LC/MS (m/z): 265 (M+H)⁺

### Intermediate 179

### Preparation of 2-(4-(chloromethyl)-1H-1,2,3-triazol-1-yl)pyridine

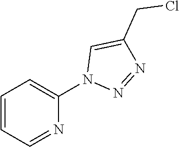 [SMILES: ClCc1cn(-c2ccccn2)nn1]

### Step A: methyl 1-(pyridin-2-yl)-1H-1,2,3-triazole-4-carboxylate

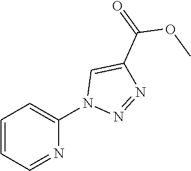

Methyl propiolate (6.67 mL, 74.9 mmol), CuSO₄ (0.399 g, 2.50 mmol), and (+)-sodium L-ascorbate (0.990 g, 5.00 mmol) were added sequentially to a mixture of 2-azidopyridine (3.00 g, 25.0 mmol) in t-BuOH (30 mL). The mixture was stirred and heated at 50° C. for 2 hours. The mixture was filtered and the filtrate was concentrated under reduced pressure. The residue was purified by silica gel chromatography (eluting ethyl acetate in petroleum ether) to afford methyl 1-(pyridin-2-yl)-1H-1,2,3-triazole-4-carboxylate. ¹H NMR (400 MHz, chloroform-d) δ 9.10 (s, 1H), 8.54 (dd, J=0.9, 4.8 Hz, 1H), 8.25 (d, J=8.2 Hz, 1H), 7.97 (dt, J=1.8, 7.8 Hz, 1H), 7.42 (ddd, J=0.8, 4.9, 7.4 Hz, 1H), 4.01 (s, 3H). LC/MS (m/z): 205 (M+H)⁺

### Step B: (1-(pyridin-2-yl)-1H-1,2,3-triazol-4-yl)methanol

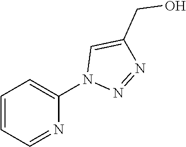 [SMILES: OCc1cn(-c2ccccn2)nn1]

LiBH₄ (133 mg, 6.12 mmol) was added to a mixture of methyl 1-(pyridin-2-yl)-1H-1,2,3-triazole-4-carboxylate (0.500 g, 2.45 mmol) in THE (10 mL) at 0° C. The mixture was stirred at room temperature for 20 hours. The mixture was quenched with water (10 mL) and extracted with EtOAc (3×15 mL). The organic layers were combined, washed with brine (10 mL), dried over anhydrous sodium sulfate, filtered, and concentrated under reduced pressure. The residue was purified by silica gel chromatography (eluting ethyl acetate in petroleum ether) to afford (1-(pyridin-2-yl)-1H-1,2,3-triazol-4-yl)methanol. ¹H NMR (400 MHz, chloroform-d) δ 8.58 (s, 1H), 8.52 (dd, J=1.0, 5.0 Hz, 1H), 8.21 (d, J=8.2 Hz, 1H), 7.97-7.91 (m, 1H), 7.37 (ddd, J=1.0, 4.9, 7.4 Hz, 1H), 4.92 (s, 2H). LC/MS (m/z): 177 (M+H)⁺

### Step C: 2-(4-(chloromethyl)-1H-1,2,3-triazol-1-yl)pyridine

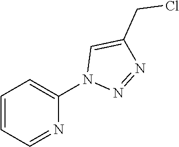 [SMILES: ClCc1cn(-c2ccccn2)nn1]

Thionyl chloride (0.186 mL, 2.55 mmol) was added to a mixture of (1-(pyridin-2-yl)-1H-1,2,3-triazol-4-yl)methanol (0.150 g, 0.851 mmol) in DCM (3 mL) at 0° C. The mixture was stirred at room temperature for 16 hours. The mixture was concentrated under reduced pressure to afford 2-(4-(chloromethyl)-1H-1,2,3-triazol-1-yl)pyridine, which was used without purification in the next step. LC/MS (m/z): 195 (M+H)⁺

### Intermediate 180

### Preparation of 2-(chloromethyl)-5-(1H-1,2,3-triazol-1-yl)pyridine

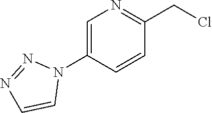 [SMILES: ClCc1ccc(-n2ccnn2)cn1]

### Step A: 2-methyl-5-(1H-1,2,3-triazol-1-yl)pyridine

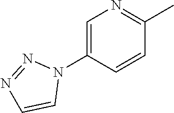

A mixture of 5-bromo-2-methylpyridine (5.00 g, 29.1 mmol), 1H-1,2,3-triazole (2.53 mL, 43.6 mmol), copper (I) iodide (0.554 g, 2.91 mmol), (S,S)-(+)-N,N′-dimethyl-1,2-cyclohexanediamine (0.620 g, 4.36 mmol) and potassium phosphate (12.3 g, 58.1 mmol) in DMF (120 mL) was stirred and heated at 100° C. for 16 hours. The mixture was quenched with water (150 mL) and extracted with EtOAc (3×150 mL). The organic layers were combined, washed with brine (150 mL), dried over anhydrous sodium sulfate, filtered, and concentrated under reduced pressure.

The residue was purified by silica gel chromatography (eluting ethyl acetate in petroleum ether) to afford 2-methyl-5-(1H-1,2,3-triazol-1-yl)pyridine. LC/MS (m/z): 161 (M+H)⁺

### Step B: 2-methyl-5-(1H-1,2,3-triazol-1-yl)pyridine 1-oxide

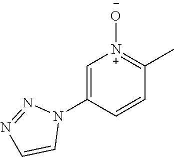

A mixture of 2-methyl-5-(1H-1,2,3-triazol-1-yl)pyridine (0.040 g, 0.25 mmol) and 3-chloroperoxybenzoic acid (47 mg, 0.28 mmol) in DCM (1 mL) was stirred at room temperature for 16 hours. The mixture was quenched with water (2 mL) and extracted with DCM (3×2 mL). The organic layers were combined, washed with brine (2 mL), dried over anhydrous sodium sulfate, filtered, and concentrated under reduced pressure to afford 2-methyl-5-(1H-1,2,3-triazol-1-yl)pyridine 1-oxide, which was used in the next step without purification.

### Step C: (5-(1H-1,2,3-triazol-1-yl)pyridin-2-yl)methanol

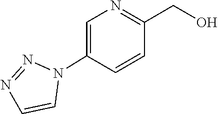 [SMILES: OCc1ccc(-n2ccnn2)cn1]

A mixture of 2-methyl-5-(1H-1,2,3-triazol-1-yl)pyridine 1-oxide (0.390 g, 2.21 mmol) and TFAA (0.688 mL, 4.87 mmol) in DCM (10 mL) was stirred at room temperature for 40 hours. The mixture was concentrated under reduced pressure and the residue was partitioned between water (10 mL) and EtOAc (10 mL). The organic layer was separated and the aqueous was re-extracted with EtOAc (3×10 mL). The organic layers were combined, washed with brine (50 mL), dried over anhydrous sodium sulfate, filtered, and concentrated under reduced pressure. The residue was purified by silica gel chromatography (eluting ethyl acetate in petroleum ether) to afford (5-(1H-1,2,3-triazol-1-yl)pyridin-2-yl)methanol. ¹H NMR (400 MHz, methanol-d₄) δ 9.02 (d, J=2.5 Hz, 1H), 8.65 (d, J=1.2 Hz, 1H), 8.34 (dd, J=2.6, 8.5 Hz, 1H), 7.95 (d, J=1.2 Hz, 1H), 7.78 (d, J=8.58 Hz, 1H), 4.79 (s, 2H). LC/MS (m/z): 177 (M+H)⁺

### Step D: 2-(chloromethyl)-5-(1H-1,2,3-triazol-1-yl)pyridine

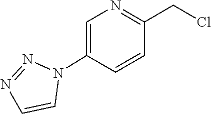 [SMILES: ClCc1ccc(-n2ccnn2)cn1]

Thionyl chloride (0.435 mL, 5.96 mmol) was added to a mixture of (5-(1H-1,2,3-triazol-1-yl)pyridin-2-yl)methanol (0.350 g, 1.99 mmol) in DCM (5 mL) at 0° C. The mixture was warmed to room temperature and stirred for 16 hours. The mixture was concentrated under reduced pressure to afford 2-(chloromethyl)-5-(1H-1,2,3-triazol-1-yl)pyridine, which was used in the next step without purification. LC/MS (m/z): 195 (M+H)⁺

### Example 1

### Preparation of 1-cyclobutyl-4-((6-(2-fluorophenyl)pyridazin-3-yl)methyl)piperazine-2,3-dione

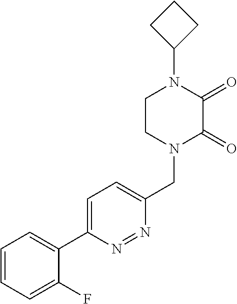 [SMILES: O=C1C(=O)N(C2CCC2)CCN1Cc1ccc(-c2ccccc2F)nn1]

A mixture of 1-((6-chloropyridazin-3-yl)methyl)-4-cyclobutylpiperazine-2,3-dione (3.50 g, 11.9 mmol), (2-fluorophenyl)boronic acid (1.75 g, 12.5 mmol), Pd(dtbpf)Cl₂ (0.45 g, 0.69 mmol), and potassium phosphate tribasic (3.78 g, 17.8 mmol) in water (10 mL) and 1,4-dioxane (50 mL) was degassed with argon for 5 minutes. The mixture was stirred and heated at 80° C. for 3 hours. The mixture was cooled to room temperature, and the organic layer was separated, dried over anhydrous Na₂SO₄, filtered, and concentrated under reduced pressure. The residue was purified by silica gel chromatography (eluting ethyl acetate in petroleum ether) to afford the product. The product was dissolved in 1,2-dichloroethane (50 mL). Sodium bisulfite (4.58 g, 44.0 mmol) was added, and the mixture was stirred and heated at 65° C. for 12 hours. The mixture was cooled to room temperature, filtered, and concentrated under reduced pressure. The residue was purified by silica gel chromatography (eluting ethyl acetate in petroleum ether) to afford 1-cyclobutyl-4-((6-(2-fluorophenyl)pyridazin-3-yl)methyl)piperazine-2,3-dione. ¹H NMR (500 MHz, chloroform-d) δ 8.15 (td, J=7.8 Hz, 1.8 Hz, 1H), 7.96 (d, J=9.0 Hz, 1H), 7.72 (d, J=8.9 Hz, 1H), 7.54-7.44 (m, 1H), 7.34 (td, J=7.6, 1.0 Hz, 1H), 7.24-7.22 (m, 1H), 5.05-4.94 (m, 3H), 3.87-3.78 (m, 2H), 3.63-3.55 (m, 2H), 2.25-2.05 (m, 4H), 1.79-1.69 (m, 2H). LC/MS (m/z): 355 (M+H)⁺

### Example 2

### Preparation of 1-(bicyclo[1.1.1]pentan-1-yl)-4-((5-(3-fluorophenyl)-1,3,4-thiadiazol-2-yl)methyl)piperazine-2,3-dione

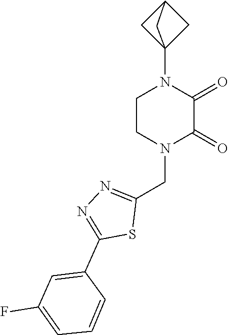 [SMILES: O=C1C(=O)N(C23CC(C2)C3)CCN1Cc1nnc(-c2cccc(F)c2)s1]

A mixture of 1-(bicyclo[1.1.1]pentan-1-yl)piperazine-2,3-dione (0.800 g, 4.44 mmol), 2-(chloromethyl)-5-(3-fluorophenyl)-1,3,4-thiadiazole (1.02 g, 4.44 mmol), and Cs₂CO₃ (1.81 g, 13.3 mmol) in DMF (25 mL) was stirred and heated at 60° C. for 12 hours. The mixture was cooled to room temperature and filtered. The filtrate was concentrated under reduced pressure, and the residue was purified by silica gel chromatography (eluting methanol in dichloromethane) to afford 1-(bicyclo[1.1.1]pentan-1-yl)-4-((5-(3-fluorophenyl)-1,3,4-thiadiazol-2-yl)methyl)piperazine-2,3-dione. ¹H NMR (500 MHz, methanol-d₄) δ 7.80-7.72 (m, 2H), 7.55 (dt, J=8.1 Hz, 5.7 Hz, 1H), 7.31 (dt, J=8.5 Hz, 2.0 Hz, 1H), 5.07 (s, 2H), 3.80-3.73 (m, 2H), 3.64-3.60 (m, 2H), 2.50 (s, 1H), 2.20 (s, 6H). LC/MS (m/z): 373 (M+H)⁺

### Example 3

### Preparation of 1-cyclopentyl-4-((6-phenylpyridazin-3-yl)methyl)piperazine-2,3-dione

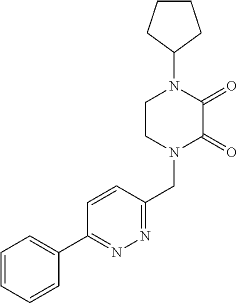 [SMILES: O=C1C(=O)N(C2CCCC2)CCN1Cc1ccc(-c2ccccc2)nn1]

A mixture of 1-((6-chloropyridazin-3-yl)methyl)-4-cyclopentylpiperazine-2,3-dione (10 mg, 0.032 mmol), Pd(dtbpf)Cl₂ (2 mg, 3 μmol), phenylboronic acid (5 mg, 0.04 mmol) and K₃PO₄ (21 mg, 0.097 mmol) in 1,4-dioxane (2 mL) was sparged with nitrogen for 5 minutes. The reaction mixture was stirred and heated to 80° C. under a nitrogen atmosphere for 16 hours. The reaction mixture was cooled to room temperature, quenched with water (10 mL), and extracted with EtOAc (3×10 mL). The organic layers were combined, washed with brine (10 mL), dried over anhydrous sodium sulfate, filtered, and concentrated under reduced pressure. The residue was purified by reverse phase HPLC (eluting acetonitrile in water, with TFA modifier) to afford 1-cyclopentyl-4-((6-phenylpyridazin-3-yl)methyl)piperazine-2,3-dione. ¹H NMR (500 MHz, chloroform-d) δ 8.07-8.03 (m, 2H), 7.92 (d, J=8.7 Hz, 1H), 7.81 (d, J=8.4 Hz, 1H), 7.57-7.51 (m, 3H), 5.00 (s, 2H), 4.89 (quin, J=8.6 Hz, 1H), 3.84-3.77 (m, 2H), 3.51-3.44 (m, 2H), 1.95-1.87 (m, 2H), 1.74-1.59 (m, 4H), 1.50-1.42 (m, 2H). LC/MS (m/z): 351 (M+H)⁺

Examples shown in Example Table 1 below, were or may be prepared according to procedures analogous to those outlined in Example 3 above using the appropriate starting materials, described in the Preparations or Intermediates above, or as obtained from commercial sources.

Example Table 1

| Example | Structure | Name | Mass [M + H]⁺ |
|---|---|---|---|
| Example 4 | 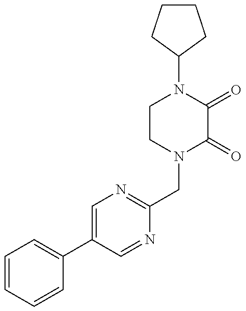 [SMILES: O=C1C(=O)N(C2CCCC2)CCN1Cc1ncc(-c2ccccc2)cn1] | 1-cyclopentyl-4-((5- phenylpyrimidin-2- yl)methyl)piperazine-2,3- dione | 351 |
| Example 5 | 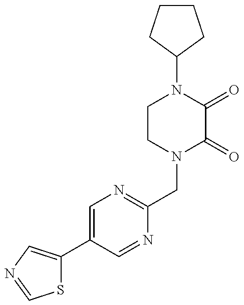 [SMILES: O=C1C(=O)N(C2CCCC2)CCN1Cc1ncc(-c2cncs2)cn1] | 1-cyclopentyl-4-((5- (thiazol-5-yl)pyrimidin-2- yl)methyl)piperazine-2,3- dione | 358 |
| Example 6 | 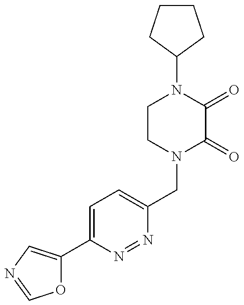 [SMILES: O=C1C(=O)N(C2CCCC2)CCN1Cc1ccc(-c2cnco2)nn1] | 1-cyclopentyl-4-((6- (oxazol-5-yl)pyridazin-3- yl)methyl)piperazine-2,3- dione | 342 |
| Example 7 | 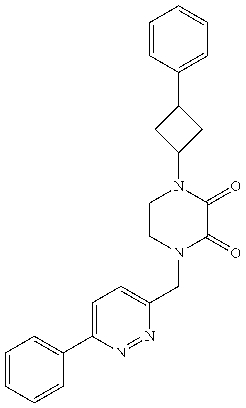 [SMILES: O=C1C(=O)N(C2CC(c3ccccc3)C2)CCN1Cc1ccc(-c2ccccc2)nn1] | 1-(3-phenylcyclobutyl)-4- ((6-phenylpyridazin-3- yl)methyl)piperazine-2,3- dione | 413 |
| Example 8 | 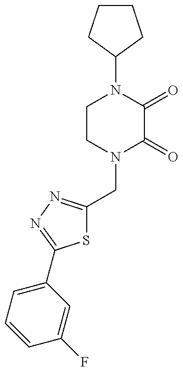 [SMILES: O=C1C(=O)N(C2CCCC2)CCN1Cc1nnc(-c2cccc(F)c2)s1] | 1-cyclopentyl-4-((5-(3- fluorophenyl)-1,3,4- thiadiazol-2- yl)methyl)piperazine-2,3- dione | 375 |
| Example 9 | 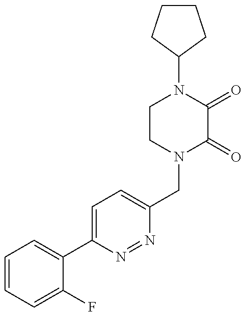 [SMILES: O=C1C(=O)N(C2CCCC2)CCN1Cc1ccc(-c2ccccc2F)nn1] | 1-cyclopentyl-4-((6-(2- fluorophenyl)pyridazin-3- yl)methyl)piperazine-2,3- dione | 369 |
| Example 10 | 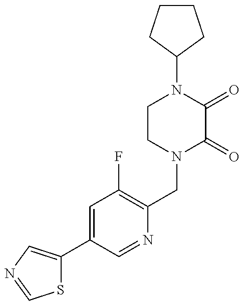 [SMILES: O=C1C(=O)N(C2CCCC2)CCN1Cc1ncc(-c2cncs2)cc1F] | 1-cyclopentyl-4-((3-fluoro- 5-(thiazol-5-yl)pyridin-2- yl)methyl)piperazine-2,3- dione | 375 |
| Example 11 | 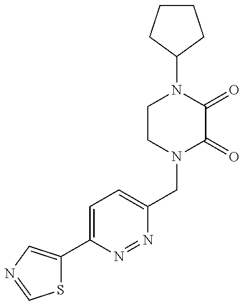 [SMILES: O=C1C(=O)N(C2CCCC2)CCN1Cc1ccc(-c2cncs2)nn1] | 1-cyclopentyl-4-((6- (thiazol-5-yl)pyridazin-3- yl)methyl)piperazine-2,3- dione | 358 |
| Example 12 | 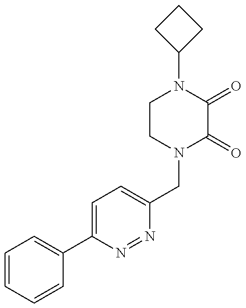 [SMILES: O=C1C(=O)N(C2CCC2)CCN1Cc1ccc(-c2ccccc2)nn1] | 1-cyclobutyl-4-((6- phenylpyridazin-3- yl)methyl)piperazine-2,3- dione | 337 |
| Example 13 | 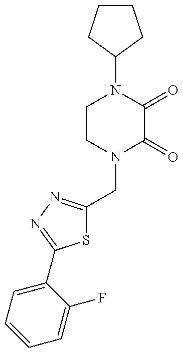 [SMILES: O=C1C(=O)N(C2CCCC2)CCN1Cc1nnc(-c2ccccc2F)s1] | 1-cyclopentyl-4-((5-(2- fluorophenyl)-1,3,4- thiadiazol-2- yl)methyl)piperazine-2,3- dione | 375 |
| Example 14 | 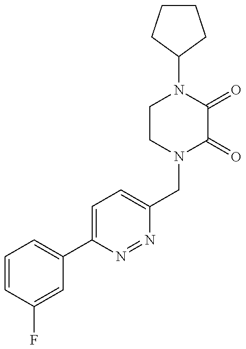 [SMILES: O=C1C(=O)N(C2CCCC2)CCN1Cc1ccc(-c2cccc(F)c2)nn1] | 1-cyclopentyl-4-((6-(3- fluorophenyl)pyridazin-3- yl)methyl)piperazine-2,3- dione | 369 |
| Example 15 | 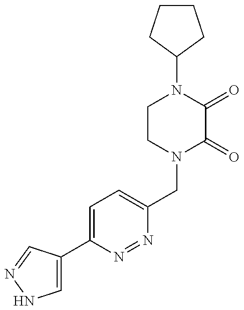 [SMILES: O=C1C(=O)N(C2CCCC2)CCN1Cc1ccc(-c2cn[nH]c2)nn1] | 1-((6-(1H-pyrazol-4- yl)pyridazin-3-yl)methyl)- 4-cyclopentylpiperazine- 2,3-dione | 341 |
| Example 16 | 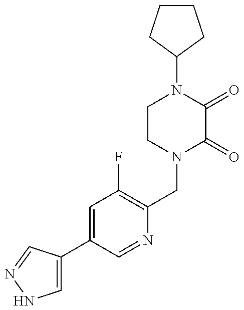 [SMILES: O=C1C(=O)N(C2CCCC2)CCN1Cc1ncc(-c2cn[nH]c2)cc1F] | 1-cyclopentyl-4-((3-fluoro- 5-(1H-pyrazol-4-yl)pyridin- 2-yl)methyl)piperazine-2,3- dione | 358 |
| Example 17 | 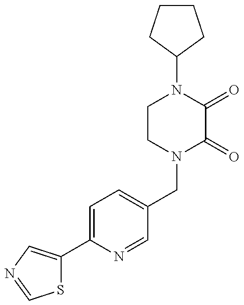 [SMILES: O=C1C(=O)N(C2CCCC2)CCN1Cc1ccc(-c2cncs2)nc1] | 1-cyclopentyl-4-((6- (thiazol-5-yl)pyridin-3- yl)methyl)piperazine-2,3- dione | 357 |
| Example 18 | 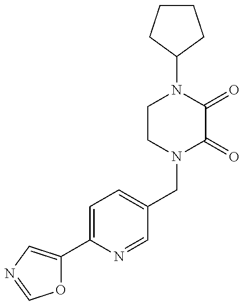 [SMILES: O=C1C(=O)N(C2CCCC2)CCN1Cc1ccc(-c2cnco2)nc1] | 1-cyclopentyl-4-((6- (oxazol-5-yl)pyridin-3- yl)methyl)piperazine-2,3- dione | 341 |
| Example 19 | 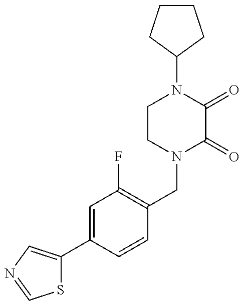 [SMILES: O=C1C(=O)N(C2CCCC2)CCN1Cc1ccc(-c2cncs2)cc1F] | 1-cyclopentyl-4-(2-fluoro- 4-(thiazol-5- yl)benzyl)piperazine-2,3- dione | 374 |
| Example 20 | 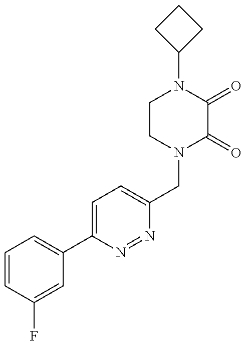 [SMILES: O=C1C(=O)N(C2CCC2)CCN1Cc1ccc(-c2cccc(F)c2)nn1] | 1-cyclobutyl-4-((6-(3- fluorophenyl)pyridazin-3- yl)methyl)piperazine-2,3- dione | 355 |
| Example 21 | 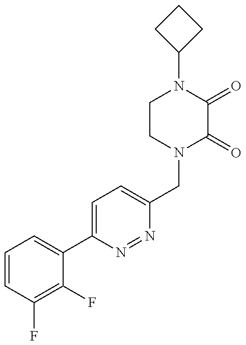 [SMILES: O=C1C(=O)N(C2CCC2)CCN1Cc1ccc(-c2cccc(F)c2F)nn1] | 1-cyclobutyl-4-((6-(2,3- difluorophenyl)pyridazin-3- yl)methyl)piperazine-2,3- dione | 373 |
| Example 22 | 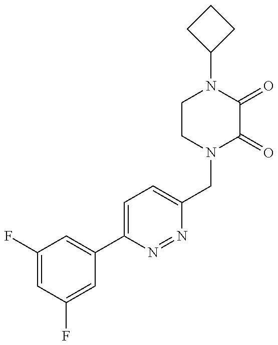 [SMILES: O=C1C(=O)N(C2CCC2)CCN1Cc1ccc(-c2cc(F)cc(F)c2)nn1] | 1-cyclobutyl-4-((6-(3,5- difluorophenyl)pyridazin-3- yl)methyl)piperazine-2,3- dione | 373 |
| Example 23 | 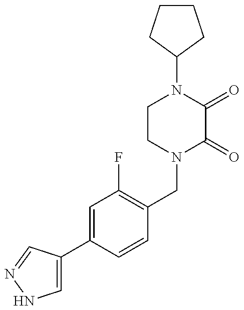 [SMILES: O=C1C(=O)N(C2CCCC2)CCN1Cc1ccc(-c2cn[nH]c2)cc1F] | 1-cyclopentyl-4-(2-fluoro- 4-(1H-pyrazol-4- yl)benzyl)piperazine-2,3- dione | 357 |
| Example 24 | 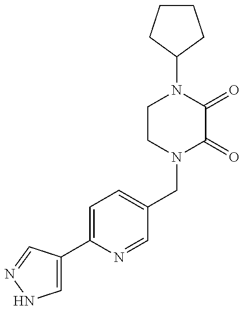 [SMILES: O=C1C(=O)N(C2CCCC2)CCN1Cc1ccc(-c2cn[nH]c2)nc1] | 1-((6-(1H-pyrazol-4- yl)pyridin-3-yl)methyl)-4- cyclopentylpiperazine-2,3- dione | 340 |
| Example 25 | 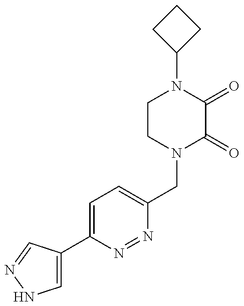 [SMILES: O=C1C(=O)N(C2CCC2)CCN1Cc1ccc(-c2cn[nH]c2)nn1] | 1-((6-(1H-pyrazol-4- yl)pyridazin-3-yl)methyl)- 4-cyclobutylpiperazine-2,3- dione | 327 |
| Example 26 | 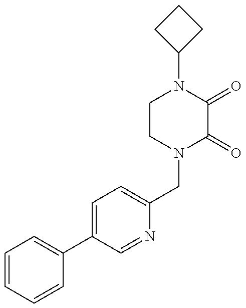 [SMILES: O=C1C(=O)N(C2CCC2)CCN1Cc1ccc(-c2ccccc2)cn1] | 1-cyclobutyl-4-((5- phenylpyridin-2- yl)methyl)piperazine-2,3- dione | 336 |
| Example 27 | 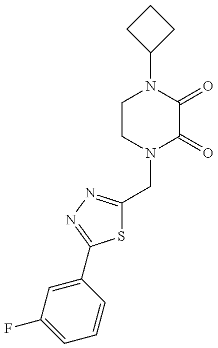 [SMILES: O=C1C(=O)N(C2CCC2)CCN1Cc1nnc(-c2cccc(F)c2)s1] | 1-cyclobutyl-4-((5-(3- fluorophenyl)-1,3,4- thiadiazol-2- yl)methyl)piperazine-2,3- dione | 361 |
| Example 28 | 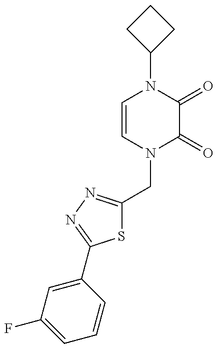 [SMILES: O=c1c(=O)n(C2CCC2)ccn1Cc1nnc(-c2cccc(F)c2)s1] | 1-cyclobutyl-4-((5-(3- fluorophenyl)-1,3,4- thiadiazol-2-yl)methyl)-1,4- dihydropyrazine-2,3-dione | 359 |
| Example 29 | 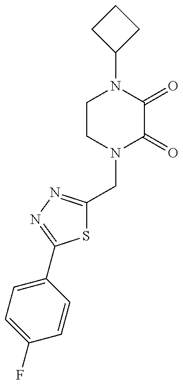 [SMILES: O=C1C(=O)N(C2CCC2)CCN1Cc1nnc(-c2ccc(F)cc2)s1] | 1-cyclobutyl-4-((5-(4- fluorophenyl)-1,3,4- thiadiazol-2- yl)methyl)piperazine-2,3- dione | 361 |
| Example 30 | 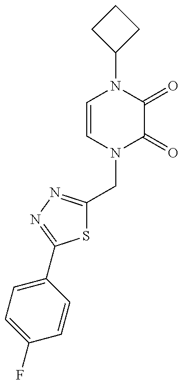 [SMILES: O=c1c(=O)n(C2CCC2)ccn1Cc1nnc(-c2ccc(F)cc2)s1] | 1-cyclobutyl-4-((5-(4- fluorophenyl)-1,3,4- thiadiazol-2-yl)methyl)-1,4- dihydropyrazine-2,3-dione | 359 |
| Example 31 | 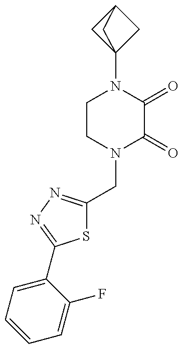 [SMILES: O=C1C(=O)N(C23CC(C2)C3)CCN1Cc1nnc(-c2ccccc2F)s1] | 1-(bicyclo[1.1.1]pentan-1- yl)-4-((5-(2-fluorophenyl)- 1,3,4-thiadiazol-2- yl)methyl)piperazine-2,3- dione | 373 |
| Example 32 | 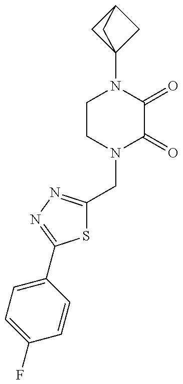 [SMILES: O=C1C(=O)N(C23CC(C2)C3)CCN1Cc1nnc(-c2ccc(F)cc2)s1] | 1-(bicyclo[1.1.1]pentan-1- yl)-4-((5-(4-fluorophenyl)- 1,3,4-thiadiazol-2- yl)methyl)piperazine-2,3- dione | 373 |
| Example 33 | 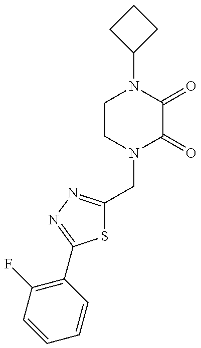 [SMILES: O=C1C(=O)N(C2CCC2)CCN1Cc1nnc(-c2ccccc2F)s1] | 1-cyclobutyl-4-((5-(2- fluorophenyl)-1,3,4- thiadiazol-2- yl)methyl)piperazine-2,3- dione | 361 |
| Example 34 | 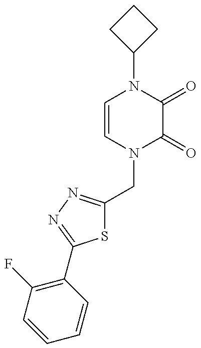 [SMILES: O=c1c(=O)n(C2CCC2)ccn1Cc1nnc(-c2ccccc2F)s1] | 1-cyclobutyl-4-((5-(2- fluorophenyl)-1,3,4- thiadiazol-2-yl)methyl)-1,4- dihydropyrazine-2,3-dione | 359 |
| Example 35 | 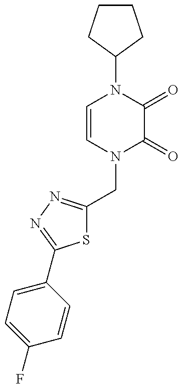 [SMILES: O=c1c(=O)n(C2CCCC2)ccn1Cc1nnc(-c2ccc(F)cc2)s1] | 1-cyclopentyl-4-((5-(4- fluorophenyl)-1,3,4- thiadiazol-2-yl)methyl)-1,4- dihydropyrazine-2,3-dione | 373 |
| Example 36 | 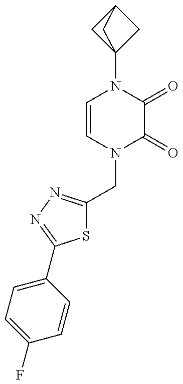 [SMILES: O=c1c(=O)n(C23CC(C2)C3)ccn1Cc1nnc(-c2ccc(F)cc2)s1] | 1-(bicyclo[1.1.1]pentan-1- yl)-4-((5-(4-fluorophenyl)- 1,3,4-thiadiazol-2- yl)methyl)-1,4- dihydropyrazine-2,3-dione | 371 |
| Example 37 | 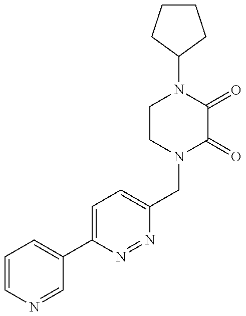 [SMILES: O=C1C(=O)N(C2CCCC2)CCN1Cc1ccc(-c2cccnc2)nn1] | 1-cyclopentyl-4-((6- (pyridin-3-yl)pyridazin-3- yl)methyl)piperazine-2,3- dione | 352 |
| Example 38 | 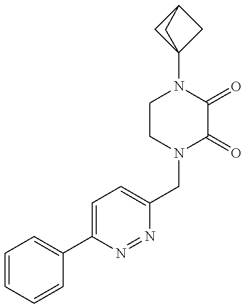 [SMILES: O=C1C(=O)N(C23CC(C2)C3)CCN1Cc1ccc(-c2ccccc2)nn1] | 1-(bicyclo[1.1.1]pentan-1- yl)-4-((6-phenylpyridazin- 3-yl)methyl)piperazine-2,3- dione | 349 |
| Example 39 | 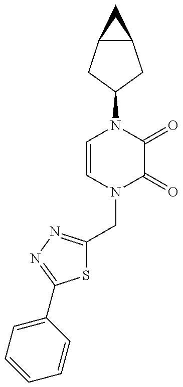 [SMILES: O=c1c(=O)n([C@H]2C[C@H]3C[C@H]3C2)ccn1Cc1nnc(-c2ccccc2)s1] | 1-((cis)- bicyclo[3.1.0]hexan-3-yl)- 4-((5-phenyl-1,3,4- thiadiazol-2-yl)methyl)-1,4- dihydropyrazine-2,3-dione | 367 |
| Example 40 | 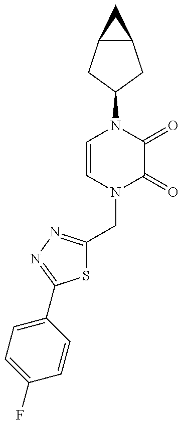 [SMILES: O=c1c(=O)n([C@H]2C[C@H]3C[C@H]3C2)ccn1Cc1nnc(-c2ccc(F)cc2)s1] | 1-((cis)- bicyclo[3.1.0]hexan-3-yl)- 4-((5-(4-fluorophenyl)- 1,3,4-thiadiazol-2- yl)methyl)piperazine-2,3- dione | 387 |
| Example 41 | 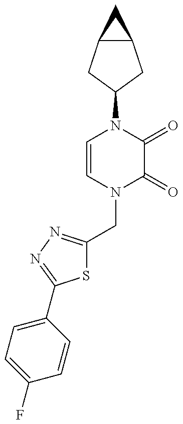 [SMILES: O=c1c(=O)n([C@H]2C[C@H]3C[C@H]3C2)ccn1Cc1nnc(-c2ccc(F)cc2)s1] | 1-((cis)- bicyclo[3.1.0]hexan-3-yl)- 4-((5-(4-fluorophenyl)- 1,3,4-thiadiazol-2- yl)methyl)-1,4- dihydropyrazine-2,3-dione | 385 |
| Example 42 | 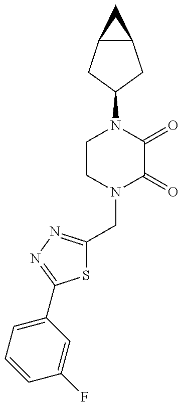 [SMILES: O=C1C(=O)N([C@H]2C[C@H]3C[C@H]3C2)CCN1Cc1nnc(-c2cccc(F)c2)s1] | 1-((cis)- bicyclo[3.1.0]hexan-3-yl)- 4-((5-(3-fluorophenyl)- 1,3,4-thiadiazol-2- yl)methyl)piperazine-2,3- dione | 387 |
| Example 43 | 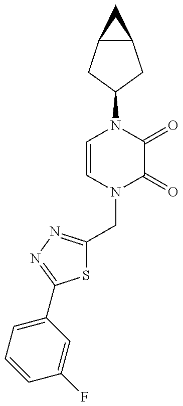 [SMILES: O=c1c(=O)n([C@H]2C[C@H]3C[C@H]3C2)ccn1Cc1nnc(-c2cccc(F)c2)s1] | 1-((cis)- bicyclo[3.1.0]hexan-3-yl)- 4-((5-(3-fluorophenyl)- 1,3,4-thiadiazol-2- yl)methyl)-1,4- dihydropyrazine-2,3-dione | 385 |
| Example 44 | 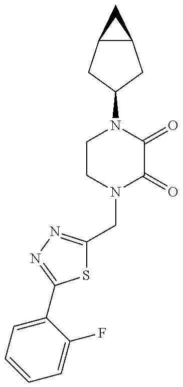 [SMILES: O=C1C(=O)N([C@H]2C[C@H]3C[C@H]3C2)CCN1Cc1nnc(-c2ccccc2F)s1] | 1-((cis)- bicyclo[3.1.0]hexan-3-yl)- 4-((5-(2-fluorophenyl)- 1,3,4-thiadiazol-2- yl)methyl)piperazine-2,3- dione | 387 |
| Example 45 | 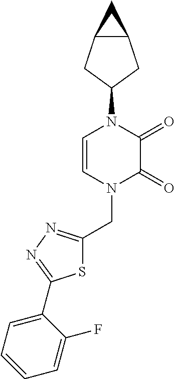 [SMILES: O=c1c(=O)n([C@H]2C[C@H]3C[C@H]3C2)ccn1Cc1nnc(-c2ccccc2F)s1] | 1-((cis)- bicyclo[3.1.0]hexan-3-yl)- 4-((5-(2-fluorophenyl)- 1,3,4-thiadiazol-2- yl)methyl)-1,4- dihydropyrazine-2,3-dione | 385 |
| Example 46 | 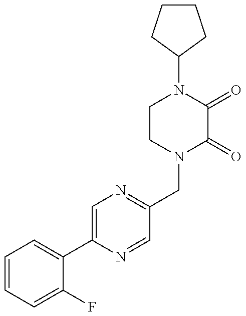 [SMILES: O=C1C(=O)N(C2CCCC2)CCN1Cc1cnc(-c2ccccc2F)cn1] | 1-cyclopentyl-4-((5-(2- fluorophenyl)pyrazin-2- yl)methyl)piperazine-2,3- dione | 369 |
| Example 47 | 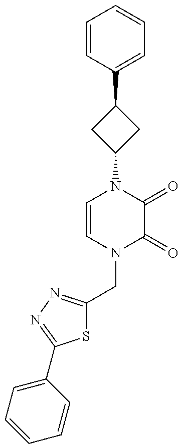 [SMILES: O=c1c(=O)n([C@H]2C[C@H](c3ccccc3)C2)ccn1Cc1nnc(-c2ccccc2)s1] | 1-((5-phenyl-1,3,4- thiadiazol-2-yl)methyl)-4- ((trans)-3- phenylcyclobutyl)-1,4- dihydropyrazine-2,3-dione | 417 |
| Example 48 | 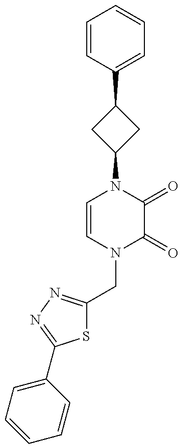 [SMILES: O=c1c(=O)n([C@H]2C[C@@H](c3ccccc3)C2)ccn1Cc1nnc(-c2ccccc2)s1] | 1-((5-phenyl-1,3,4- thiadiazol-2-yl)methyl)-4- ((cis)-3-phenylcyclobutyl)- 1,4-dihydropyrazine-2,3- dione | 417 |
| Example 49 | 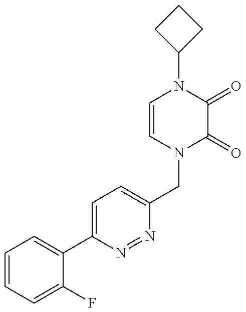 [SMILES: O=c1c(=O)n(C2CCC2)ccn1Cc1ccc(-c2ccccc2F)nn1] | 1-cyclobutyl-4-((6-(2- fluorophenyl)pyridazin-3- yl)methyl)-1,4- dihydropyrazine-2,3-dione | 353 |
| Example 50 | 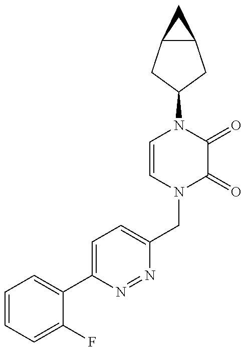 [SMILES: O=c1c(=O)n([C@H]2C[C@H]3C[C@H]3C2)ccn1Cc1ccc(-c2ccccc2F)nn1] | 1-((cis)- bicyclo[3.1.0]hexan-3-yl)- 4-((6-(2- fluorophenyl)pyridazin-3- yl)methyl)-1,4- dihydropyrazine-2,3-dione | 379 |
| Example 51 | 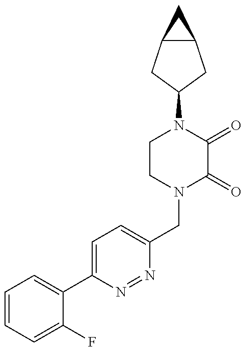 [SMILES: O=C1C(=O)N([C@H]2C[C@H]3C[C@H]3C2)CCN1Cc1ccc(-c2ccccc2F)nn1] | 1-((1R,3r,5S)- bicyclo[3.1.0]hexan-3-yl)- 4-((6-(2- fluorophenyl)pyridazin-3- yl)methyl)piperazine-2,3- dione | 381 |
| Example 52 | 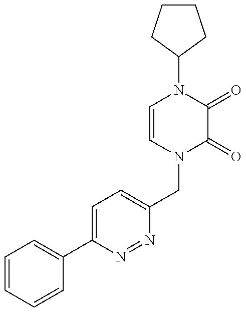 [SMILES: O=c1c(=O)n(C2CCCC2)ccn1Cc1ccc(-c2ccccc2)nn1] | 1-cyclopentyl-4-((6- phenylpyridazin-3- yl)methyl)-1,4- dihydropyrazine-2,3-dione | 349 |
| Example 53 | 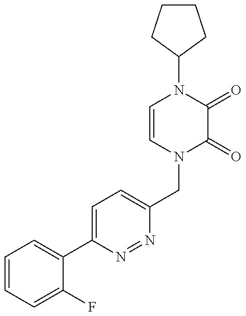 [SMILES: O=c1c(=O)n(C2CCCC2)ccn1Cc1ccc(-c2ccccc2F)nn1] | 1-cyclopentyl-4-((6-(2- fluorophenyl)pyridazin-3- yl)methyl)-1,4- dihydropyrazine-2,3-dione | 367 |
| Example 54 | 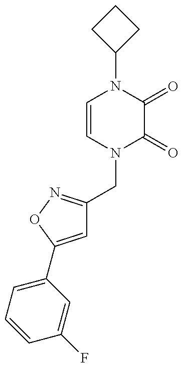 [SMILES: O=c1c(=O)n(C2CCC2)ccn1Cc1cc(-c2cccc(F)c2)on1] | 1-cyclobutyl-4-((5-(3- fluorophenyl)isoxazol-3- yl)methyl)-1,4- dihydropyrazine-2,3-dione | 342 |
| Example 55 | 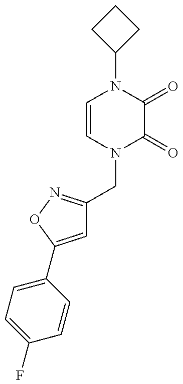 [SMILES: O=c1c(=O)n(C2CCC2)ccn1Cc1cc(-c2ccc(F)cc2)on1] | 1-cyclobutyl-4-((5-(4- fluorophenyl)isoxazol-3- yl)methyl)-1,4- dihydropyrazine-2,3-dione | 342 |
| Example 56 | 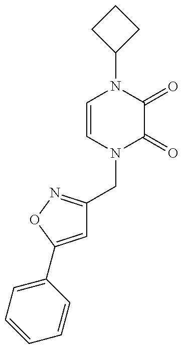 [SMILES: O=c1c(=O)n(C2CCC2)ccn1Cc1cc(-c2ccccc2)on1] | 1-cyclobutyl-4-((5- phenylisoxazol-3- yl)methyl)-1,4- dihydropyrazine-2,3-dione | 324 |
| Example 57 | 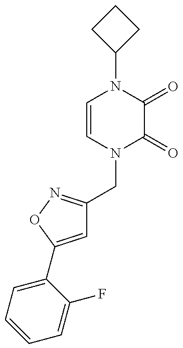 [SMILES: O=c1c(=O)n(C2CCC2)ccn1Cc1cc(-c2ccccc2F)on1] | 1-cyclobutyl-4-((5-(2- fluorophenyl)isoxazol-3- yl)methyl)-1,4- dihydropyrazine-2,3-dione | 342 |
| Example 58 | 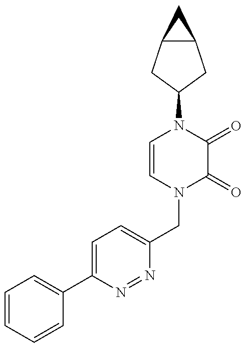 [SMILES: O=c1c(=O)n([C@H]2C[C@H]3C[C@H]3C2)ccn1Cc1ccc(-c2ccccc2)nn1] | 1-((cis)- bicyclo[3.1.0]hexan-3-yl)- 4-((6-phenylpyridazin-3- yl)methyl)-1,4- dihydropyrazine-2,3-dione | 361 |
| Example 59 | 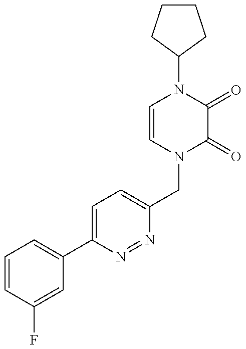 [SMILES: O=c1c(=O)n(C2CCCC2)ccn1Cc1ccc(-c2cccc(F)c2)nn1] | 1-cyclopentyl-4-((6-(3- fluorophenyl)pyridazin-3- yl)methyl)-1,4- dihydropyrazine-2,3-dione | 367 |
| Example 60 | 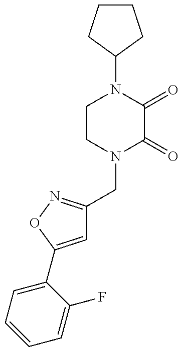 [SMILES: O=C1C(=O)N(C2CCCC2)CCN1Cc1cc(-c2ccccc2F)on1] | 1-cyclopentyl-4-((5-(2- fluorophenyl)isoxazol-3- yl)methyl)piperazine-2,3- dione | 358 |
| Example 61 | 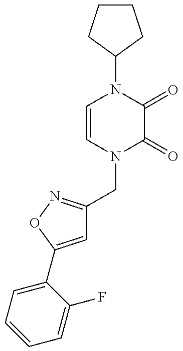 [SMILES: O=c1c(=O)n(C2CCCC2)ccn1Cc1cc(-c2ccccc2F)on1] | 1-cyclopentyl-4-((5-(2- fluorophenyl)isoxazol-3- yl)methyl)-1,4- dihydropyrazine-2,3-dione | 356 |
| Example 62 | 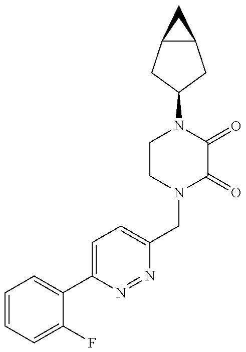 [SMILES: O=C1C(=O)N([C@H]2C[C@H]3C[C@H]3C2)CCN1Cc1ccc(-c2ccccc2F)nn1] | 1-((cis)- bicyclo[3.1.0]hexan-3-yl)- 4-((6-(2- fluorophenyl)pyridazin-3- yl)methyl)piperazine-2,3- dione | 381 |
| Example 63 | 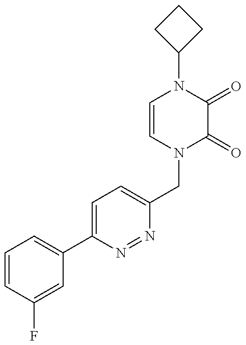 [SMILES: O=c1c(=O)n(C2CCC2)ccn1Cc1ccc(-c2cccc(F)c2)nn1] | 1-cyclobutyl-4-((6-(3- fluorophenyl)pyridazin-3- yl)methyl)-1,4- dihydropyrazine-2,3-dione | 353 |
| Example 64 | 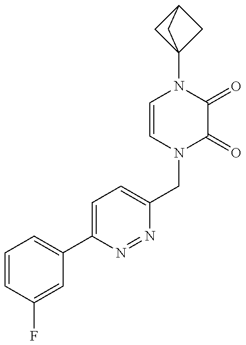 [SMILES: O=c1c(=O)n(C23CC(C2)C3)ccn1Cc1ccc(-c2cccc(F)c2)nn1] | 1-(bicyclo[1.1.1]pentan-1- yl)-4-((6-(3- fluorophenyl)pyridazin-3- yl)methyl)-1,4- dihydropyrazine-2,3-dione | 365 |
| Example 65 | 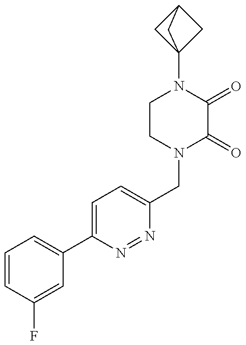 [SMILES: O=C1C(=O)N(C23CC(C2)C3)CCN1Cc1ccc(-c2cccc(F)c2)nn1] | 1-(bicyclo[1.1.1]pentan-1- yl)-4-((6-(3- fluorophenyl)pyridazin-3- yl)methyl)piperazine-2,3- dione | 367 |
| Example 66 | 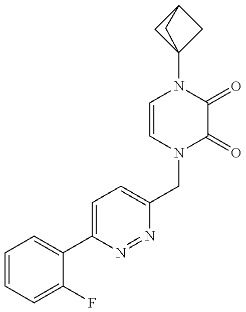 [SMILES: O=c1c(=O)n(C23CC(C2)C3)ccn1Cc1ccc(-c2ccccc2F)nn1] | 1-(bicyclo[1.1.1]pentan-1- yl)-4-((6-(2- fluorophenyl)pyridazin-3- yl)methyl)piperazine-2,3- dione | 367 |
| Example 67 | 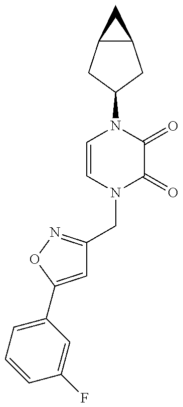 [SMILES: O=c1c(=O)n([C@H]2C[C@H]3C[C@H]3C2)ccn1Cc1cc(-c2cccc(F)c2)on1] | 1-((cis)- bicyclo[3.1.0]hexan-3-yl)- 4-((5-(3- fluorophenyl)isoxazol-3- yl)methyl)-1,4- dihydropyrazine-2,3-dione | 368 |
| Example 68 | 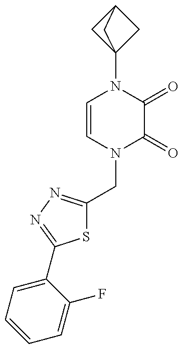 [SMILES: O=c1c(=O)n(C23CC(C2)C3)ccn1Cc1nnc(-c2ccccc2F)s1] | 1-(bicyclo[1.1.1]pentan-1- yl)-4-((5-(2-fluorophenyl)- 1,3,4-thiadiazol-2- yl)methyl)-1,4- dihydropyrazine-2,3-dione | 371 |
| Example 69 | 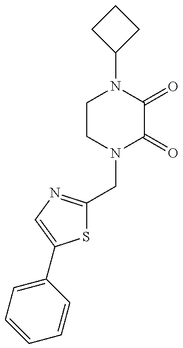 [SMILES: O=C1C(=O)N(C2CCC2)CCN1Cc1ncc(-c2ccccc2)s1] | 1-cyclobutyl-4-((5- phenylthiazol-2- yl)methyl)piperazine-2,3- dione | 342 |
| Example 70 | 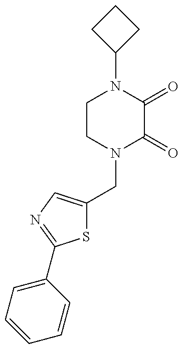 [SMILES: O=C1C(=O)N(C2CCC2)CCN1Cc1cnc(-c2ccccc2)s1] | 1-cyclobutyl-4-((2- phenylthiazol-5- yl)methyl)piperazine-2,3- dione | 342 |
| Example 71 | 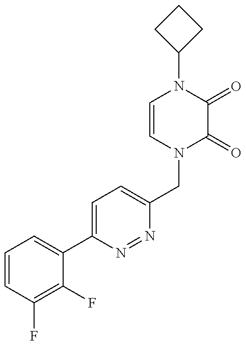 [SMILES: O=c1c(=O)n(C2CCC2)ccn1Cc1ccc(-c2cccc(F)c2F)nn1] | 1-cyclobutyl-4-((6-(2,3- difluorophenyl)pyridazin-3- yl)methyl)-1,4- dihydropyrazine-2,3-dione | 371 |
| Example 72 | 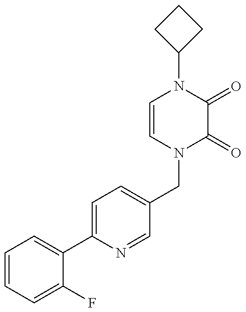 [SMILES: O=c1c(=O)n(C2CCC2)ccn1Cc1ccc(-c2ccccc2F)nc1] | 1-cyclobutyl-4-((6-(2- fluorophenyl)pyridin-3- yl)methyl)-1,4- dihydropyrazine-2,3-dione | 352 |
| Example 73 | 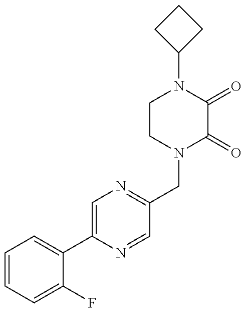 [SMILES: O=C1C(=O)N(C2CCC2)CCN1Cc1cnc(-c2ccccc2F)cn1] | 1-cyclobutyl-4-((5-(2- fluorophenyl)pyrazin-2- yl)methyl)piperazine-2,3- dione | 355 |
| Example 74 | 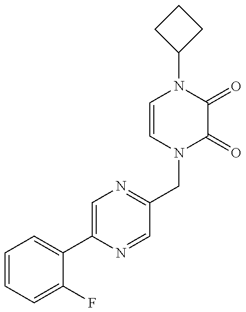 [SMILES: O=c1c(=O)n(C2CCC2)ccn1Cc1cnc(-c2ccccc2F)cn1] | 1-cyclobutyl-4-((5-(2- fluorophenyl)pyrazin-2- yl)methyl)-1,4- dihydropyrazine-2,3-dione | 353 |
| Example 75 | 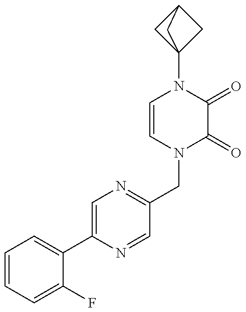 [SMILES: O=c1c(=O)n(C23CC(C2)C3)ccn1Cc1cnc(-c2ccccc2F)cn1] | 1-(bicyclo[1.1.1]pentan-1- yl)-4-((5-(2- fluorophenyl)pyrazin-2- yl)methyl)-1,4- dihydropyrazine-2,3-dione | 365 |
| Example 76 | 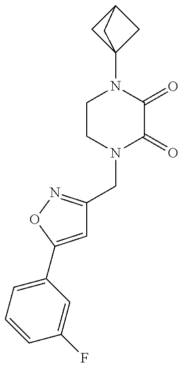 [SMILES: O=C1C(=O)N(C23CC(C2)C3)CCN1Cc1cc(-c2cccc(F)c2)on1] | 1-(bicyclo[1.1.1]pentan-1- yl)-4-((5-(3- fluorophenyl)isoxazol-3- yl)methyl)piperazine-2,3- dione | 356 |
| Example 77 | 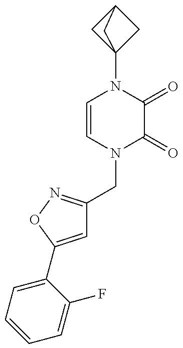 [SMILES: O=c1c(=O)n(C23CC(C2)C3)ccn1Cc1cc(-c2ccccc2F)on1] | 1-(bicyclo[1.1.1]pentan-1- yl)-4-((5-(2- fluorophenyl)isoxazol-3- yl)methyl)-1,4- dihydropyrazine-2,3-dione | 354 |
| Example 78 | 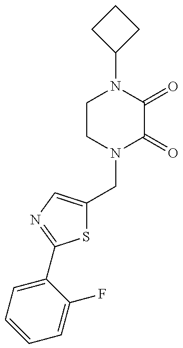 [SMILES: O=C1C(=O)N(C2CCC2)CCN1Cc1cnc(-c2ccccc2F)s1] | 1-cyclobutyl-4-((2-(2- fluorophenyl)thiazol-5- yl)methyl)piperazine-2,3- dione | 360 |
| Example 79 | 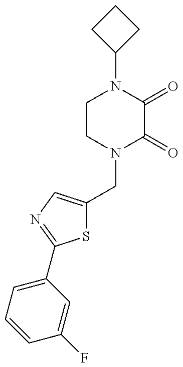 [SMILES: O=C1C(=O)N(C2CCC2)CCN1Cc1cnc(-c2cccc(F)c2)s1] | 1-cyclobutyl-4-((2-(3- fluorophenyl)thiazol-5- yl)methyl)piperazine-2,3- dione | 360 |
| Example 80 | 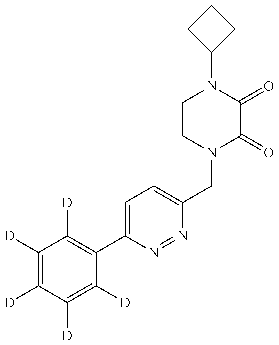 [SMILES: [2H]c1c([2H])c([2H])c(-c2ccc(CN3CCN(C4CCC4)C(=O)C3=O)nn2)c([2H])c1[2H]] | 1-cyclobutyl-4-((6-(phenyl- d₅)pyridazin-3- yl)methyl)piperazine-2,3- dione | 342 |
| Example 81 | 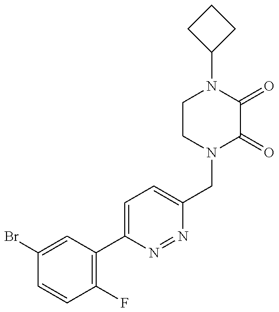 [SMILES: O=C1C(=O)N(C2CCC2)CCN1Cc1ccc(-c2cc(Br)ccc2F)nn1] | 1-((6-(5-bromo-2- fluorophenyl)pyridazin-3- yl)methyl)-4- cyclobutylpiperazine-2,3- dione | 433, 435 |
| Example 82 | 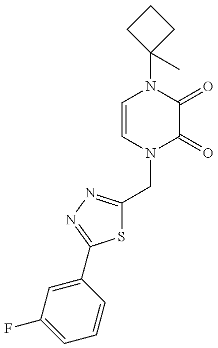 [SMILES: CC1(n2ccn(Cc3nnc(-c4cccc(F)c4)s3)c(=O)c2=O)CCC1] | 1-((5-(3-fluorophenyl)- 1,3,4-thiadiazol-2- yl)methyl)-4-(1- methylcyclobutyl)-1,4- dihydropyrazine-2,3-dione | 373 |
| Example 83 | 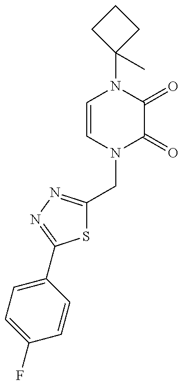 [SMILES: CC1(n2ccn(Cc3nnc(-c4ccc(F)cc4)s3)c(=O)c2=O)CCC1] | 1-((5-(4-fluorophenyl)- 1,3,4-thiadiazol-2- yl)methyl)-4-(1- methylcyclobutyl)-1,4- dihydropyrazine-2,3-dione | 373 |
| Example 84 | 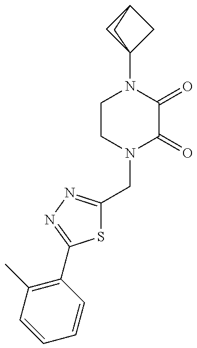 [SMILES: Cc1ccccc1-c1nnc(CN2CCN(C34CC(C3)C4)C(=O)C2=O)s1] | 1-(bicyclo[1.1.1]pentan-1- yl)-4-((5-(o-tolyl)-1,3,4- thiadiazol-2- yl)methyl)piperazine-2,3- dione | 369 |
| Example 85 | 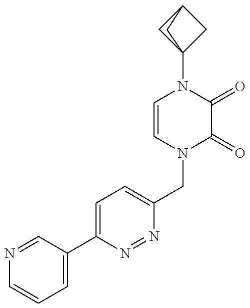 [SMILES: O=c1c(=O)n(C23CC(C2)C3)ccn1Cc1ccc(-c2cccnc2)nn1] | 1-(bicyclo[1.1.1]pentan-1- yl)-4-((6-(pyridin-3- yl)pyridazin-3-yl)methyl)- 1,4-dihydropyrazine-2,3- dione | 348 |
| Example 86 | 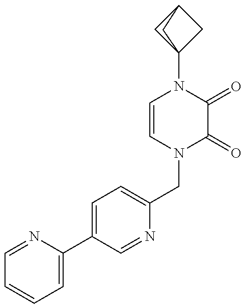 [SMILES: O=c1c(=O)n(C23CC(C2)C3)ccn1Cc1ccc(-c2ccccn2)cn1] | 1-([2,3′-bipyridin]-6′- ylmethyl)-4- (bicyclo[1.1.1]pentan-1-yl)- 1,4-dihydropyrazine-2,3- dione | 347 |
| Example 87 | 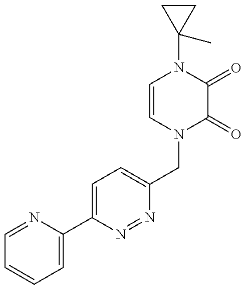 [SMILES: CC1(n2ccn(Cc3ccc(-c4ccccn4)nn3)c(=O)c2=O)CC1] | 1-(1-methylcyclopropyl)-4- ((6-(pyridin-2-yl)pyridazin- 3-yl)methyl)-1,4- dihydropyrazine-2,3-dione | 336 |
| Example 88 | 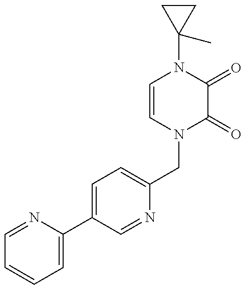 [SMILES: CC1(n2ccn(Cc3ccc(-c4ccccn4)cn3)c(=O)c2=O)CC1] | 1-([2,3′-bipyridin]-6′- ylmethyl)-4-(1- methylcyclopropyl)-1,4- dihydropyrazine-2,3-dione | 335 |
| Example 89 | 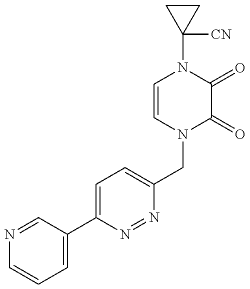 [SMILES: N#CC1(n2ccn(Cc3ccc(-c4cccnc4)nn3)c(=O)c2=O)CC1] | 1-(2,3-dioxo-4-((6-(pyridin- 3-yl)pyridazin-3- yl)methyl)-3,4- dihydropyrazin-1(2H)- yl)cyclopropane-1- carbonitrile | 347 |
| Example 90 | 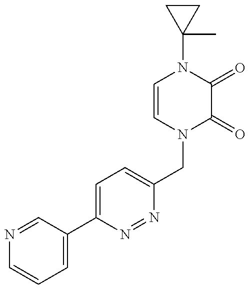 [SMILES: CC1(n2ccn(Cc3ccc(-c4cccnc4)nn3)c(=O)c2=O)CC1] | 1-(1-methylcyclopropyl)-4- ((6-(pyridin-3-yl)pyridazin- 3-yl)methyl)-1,4- dihydropyrazine-2,3-dione | 336 |
| Example 91 | 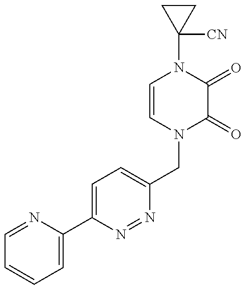 [SMILES: N#CC1(n2ccn(Cc3ccc(-c4ccccn4)nn3)c(=O)c2=O)CC1] | 1-(2,3-dioxo-4-((6-(pyridin- 2-yl)pyridazin-3- yl)methyl)-3,4- dihydropyrazin-1(2H)- yl)cyclopropane-1- carbonitrile | 347 |
| Example 92 | 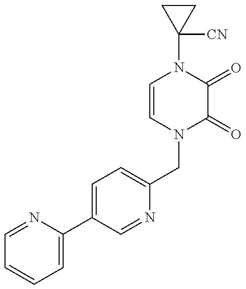 [SMILES: N#CC1(n2ccn(Cc3ccc(-c4ccccn4)cn3)c(=O)c2=O)CC1] | 1-(4-([2,3′-bipyridin]-6′- ylmethyl)-2,3-dioxo-3,4- dihydropyrazin-1(2H)- yl)cyclopropane-1- carbonitrile | 346 |
| Example 93 | 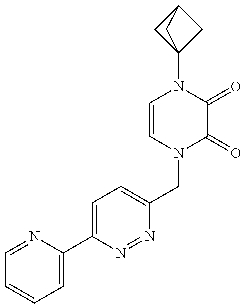 [SMILES: O=c1c(=O)n(C23CC(C2)C3)ccn1Cc1ccc(-c2ccccn2)nn1] | 1-(bicyclo[1.1.1]pentan-1- yl)-4-((6-(pyridin-2- yl)pyridazin-3-yl)methyl)- 1,4-dihydropyrazine-2,3- dione | 348 |
| Example 94 | 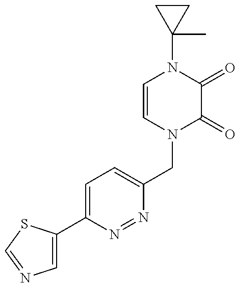 [SMILES: CC1(n2ccn(Cc3ccc(-c4cncs4)nn3)c(=O)c2=O)CC1] | 1-(1-methylcyclopropyl)-4- ((6-(thiazol-5-yl)pyridazin- 3-yl)methyl)-1,4- dihydropyrazine-2,3-dione | 342 |
| Example 95 | 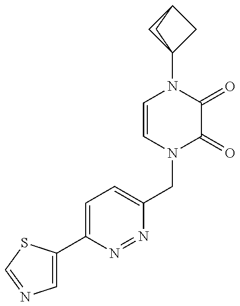 [SMILES: O=c1c(=O)n(C23CC(C2)C3)ccn1Cc1ccc(-c2cncs2)nn1] | 1-(bicyclo[1.1.1]pentan-1- yl)-4-((6-(thiazol-5- yl)pyridazin-3-yl)methyl)- 1,4-dihydropyrazine-2,3- dione | 354 |

### Examples 96A and 96B

### Preparation of trans-1-(3-phenylcyclobutyl)-4-((6-phenylpyridazin-3-yl)methyl)piperazine-2,3-dione and cis-1-(3-phenylcyclobutyl)-4-((6-phenylpyridazin-3-yl)methyl)piperazine-2,3-dione

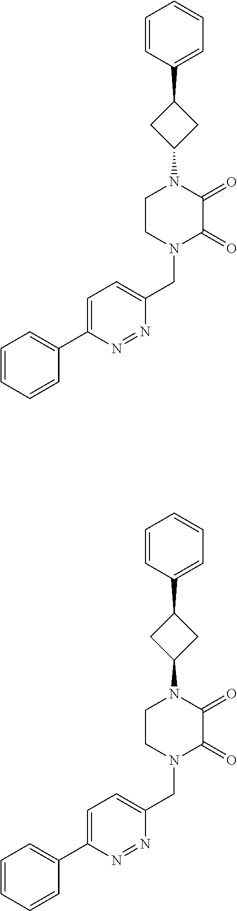 [SMILES: O=C1C(=O)N([C@H]2C[C@H](c3ccccc3)C2)CCN1Cc1ccc(-c2ccccc2)nn1 ; O=C1C(=O)N([C@H]2C[C@@H](c3ccccc3)C2)CCN1Cc1ccc(-c2ccccc2)nn1]

The isomeric mixture of 1-(3-phenylcyclobutyl)-4-((6-phenylpyridazin-3-yl)methyl)piperazine-2,3-dione was resolved by chiral SFC purification (SJ 21×250 mm column, 45% MeOH w/ 0.1% NH₄₀H as cosolvent) to afford trans-1-(3-phenylcyclobutyl)-4-((6-phenylpyridazin-3-yl)methyl)piperazine-2,3-dione as the first eluting peak and cis-1-(3-phenylcyclobutyl)-4-((6-phenylpyridazin-3-yl)methyl)piperazine-2,3-dione as the second eluting peak.

Peak 1: ¹H NMR (500 MHz, DMSO-d₆) δ 8.23 (d, J=8.8 Hz, 1H), 8.19-8.13 (m, 2H), 7.77 (d, J=8.8 Hz, 1H), 7.61-7.52 (m, 3H), 7.38-7.36 (m, 4H), 7.25-7.21 (m, 1H), 5.01 (p, J=8.3 Hz, 1H), 4.94 (s, 2H), 3.79-3.75 (m, 4H), 3.53-3.48 (m, 1H), 2.79-2.69 (m, 2H), 2.37-2.33 (m, 2H). LC/MS (m/z): 413 (M+H)⁺

Peak 2: ¹H NMR (499 MHz, DMSO-d₆) δ 8.23 (d, J=8.8 Hz, 1H), 8.16-8.14 (m, 2H), 7.76 (d, J=8.9 Hz, 1H), 7.61-7.52 (m, 3H), 7.34-7.29 (m, 4H), 7.22-7.19 (m, 1H), 4.93 (s, 2H), 4.79-4.71 (m, 1H), 3.74-3.66 (m, 4H), 3.23-3.17 (m, 1H), 2.64-2.51 m, 2H), 2.33-2.24 (m, 2H). LC/MS (m/z): 413 (M+H)⁺

### Example 97

### Preparation of 1-cyclopentyl-4-((6-phenylpyridin-3-yl)methyl)piperazine-2,3-dione

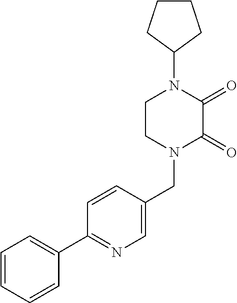 [SMILES: O=C1C(=O)N(C2CCCC2)CCN1Cc1ccc(-c2ccccc2)nc1]

NaH (25 mg, 0.62 mmol, 60% dispersion in mineral oil) was added to a mixture of 1-cyclopentylpiperazine-2,3-dione (75 mg, 0.42 mmol) and 5-(bromomethyl)-2-phenylpyridine (102 mg, 0.42 mmol) in DMF (4 mL) at 0° C. The reaction mixture was warmed to room temperature and stirred for 12 hours. The mixture was quenched with water (0.5 mL) and filtered. The filtrate was concentrated under reduced pressure, and the residue was purified by silica gel chromatography (eluting methanol in dichloromethane) to afford 1-cyclopentyl-4-((6-phenylpyridin-3-yl)methyl)piperazine-2,3-dione. ¹H NMR (400 MHz, methanol-d₄) δ 8.61 (s, 1H), 7.98-7.92 (m, 2H), 7.91-7.82 (m, 2H), 7.53-7.41 (m, 3H), 4.83-4.77 (m, 1H), 4.75 (s, 2H), 3.62-3.57 (m, 2H), 3.56-3.50 (m, 2H), 1.97-1.84 (m, 2H), 1.81-1.70 (m, 2H), 1.68-1.52 (m, 4H). LC/MS (m/z): 350 (M+H)⁺

Examples shown in Example Table 2 below, were or may be prepared according to procedures analogous to those outlined in Example 97 above using the appropriate starting materials, described in the Preparations or Intermediates above, or as obtained from commercial sources.

EXAMPLE TABLE 2

| | | | Mass |
|---|---|---|---|
| Example | Structure | Name | [M + H]⁺ |
| Example 98 | 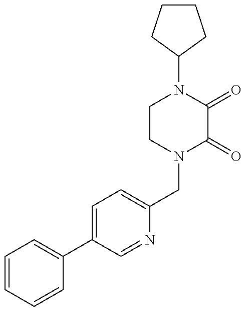 [SMILES: O=C1C(=O)N(C2CCCC2)CCN1Cc1ccc(-c2ccccc2)cn1] | 1-cyclopentyl-4-((5- phenylpyridin-2- yl)methyl)piperazine-2,3- dione | 350 |
| Example 99 (Peak 1) | 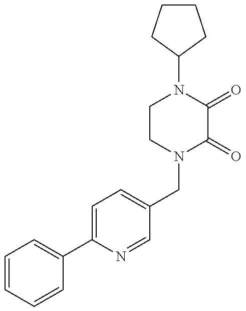 [SMILES: O=C1C(=O)N(C2CCCC2)CCN1Cc1ccc(-c2ccccc2)nc1] | (R or S)-1-cyclopentyl-4-(1- (6-phenylpyridin-3- yl)ethyl)piperazine-2,3-dione | 364 |
| Example 100 | 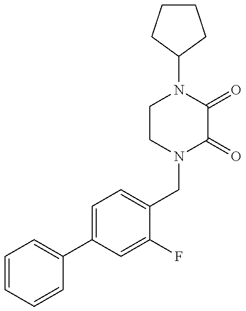 [SMILES: O=C1C(=O)N(C2CCCC2)CCN1Cc1ccc(-c2ccccc2)cc1F] | 1-cyclopentyl-4-((3-fluoro- [1,1′-biphenyl]-4- yl)methyl)piperazine-2,3- dione | 367 |
| Example 101 | 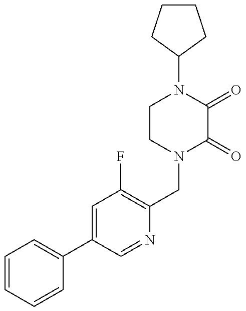 [SMILES: O=C1C(=O)N(C2CCCC2)CCN1Cc1ncc(-c2ccccc2)cc1F] | 1-cyclopentyl-4-((3-fluoro-5- phenylpyridin-2- yl)methyl)piperazine-2,3- dione | 368 |
| Example 102 | 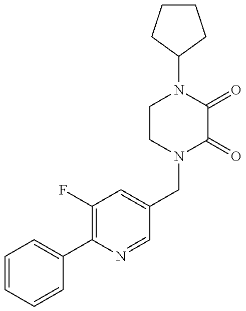 [SMILES: O=C1C(=O)N(C2CCCC2)CCN1Cc1cnc(-c2ccccc2)c(F)c1] | 1-cyclopentyl-4-((5-fluoro-6- phenylpyridin-3- yl)methyl)piperazine-2,3- dione | 368 |
| Example 103 | 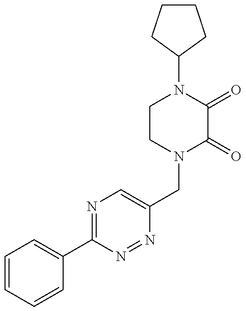 [SMILES: O=C1C(=O)N(C2CCCC2)CCN1Cc1cnc(-c2ccccc2)nn1] | 1-cyclopentyl-4-((3-phenyl- 1,2,4-triazin-6- yl)methyl)piperazine-2,3- dione | 352 |
| Example 104 | 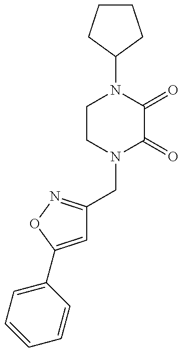 [SMILES: O=C1C(=O)N(C2CCCC2)CCN1Cc1cc(-c2ccccc2)on1] | 1-cyclopentyl-4-((5- phenylisoxazol-3- yl)methyl)piperazine-2,3- dione | 340 |
| Example 105 | 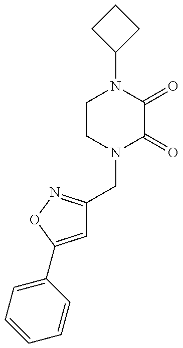 [SMILES: O=C1C(=O)N(C2CCC2)CCN1Cc1cc(-c2ccccc2)on1] | 1-cyclobutyl-4-((5- phenylisoxazol-3- yl)methyl)piperazine-2,3- dione | 326 |
| Example 106 | 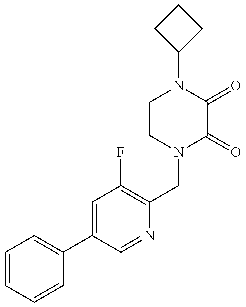 [SMILES: O=C1C(=O)N(C2CCC2)CCN1Cc1ncc(-c2ccccc2)cc1F] | 1-cyclobutyl-4-((3-fluoro-5- phenylpyridin-2- yl)methyl)piperazine-2,3- dione | 354 |
| Example 107 | 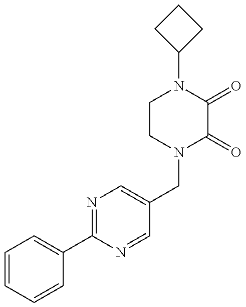 [SMILES: O=C1C(=O)N(C2CCC2)CCN1Cc1cnc(-c2ccccc2)nc1] | 1-cyclobutyl-4-((2- phenylpyrimidin-5- yl)methyl)piperazine-2,3- dione | 337 |
| Example 108 | 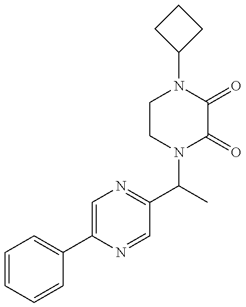 [SMILES: CC(c1cnc(-c2ccccc2)cn1)N1CCN(C2CCC2)C(=O)C1=O] | 1-cyclobutyl-4-(1-(5- phenylpyrazin-2- yl)ethyl)piperazine-2,3-dione | 351 |
| Example 109 | 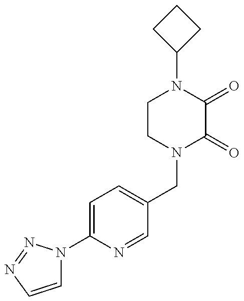 [SMILES: O=C1C(=O)N(C2CCC2)CCN1Cc1ccc(-n2ccnn2)nc1] | 1-((6-(1H-1,2,3-triazol-1- yl)pyridin-3-yl)methyl)-4- cyclobutylpiperazine-2,3- dione | 327 |
| Example 110 | 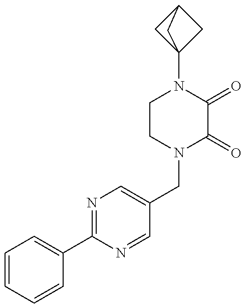 [SMILES: O=C1C(=O)N(C23CC(C2)C3)CCN1Cc1cnc(-c2ccccc2)nc1] | 1-(bicyclo[1.1.1]pentan-1-yl)- 4-((2-phenylpyrimidin-5- yl)methyl)piperazine-2,3- dione | 349 |
| Example 111 | 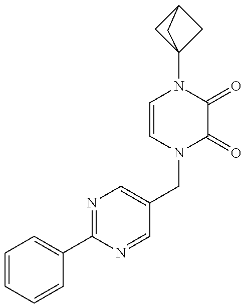 [SMILES: O=c1c(=O)n(C23CC(C2)C3)ccn1Cc1cnc(-c2ccccc2)nc1] | 1-(bicyclo[1.1.1]pentan-1-yl)- 4-((2-phenylpyrimidin-5- yl)methyl)-1,4- dihydropyrazine-2,3-dione | 347 |
| Example 112 | 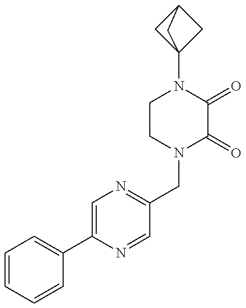 [SMILES: O=C1C(=O)N(C23CC(C2)C3)CCN1Cc1cnc(-c2ccccc2)cn1] | 1-(bicyclo[1.1.1]pentan-1-yl)- 4-((5-phenylpyrazin-2- yl)methyl)piperazine-2,3- dione | 349 |
| Example 113 | 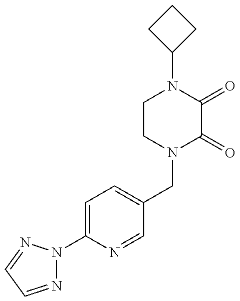 [SMILES: O=C1C(=O)N(C2CCC2)CCN1Cc1ccc(-n2nccn2)nc1] | 1-((6-(2H-1,2,3-triazol-2- yl)pyridin-3-yl)methyl)-4- cyclobutylpiperazine-2,3- dione | 327 |
| Example 114 | 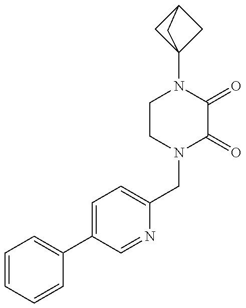 [SMILES: O=C1C(=O)N(C23CC(C2)C3)CCN1Cc1ccc(-c2ccccc2)cn1] | 1-(bicyclo[1.1.1]pentan-1-yl)- 4-((5-phenylpyridin-2- yl)methyl)piperazine-2,3- dione | 348 |
| Example 115 | 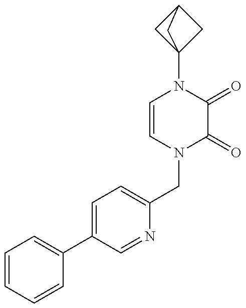 [SMILES: O=c1c(=O)n(C23CC(C2)C3)ccn1Cc1ccc(-c2ccccc2)cn1] | 1-(bicyclo[1.1.1]pentan-1-yl)- 4-((5-phenylpyridin-2- yl)methyl)-1,4- dihydropyrazine-2,3-dione | 346 |
| Example 116 | 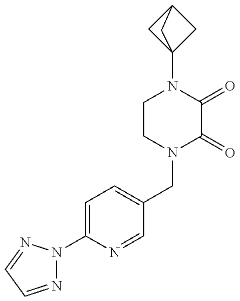 [SMILES: O=C1C(=O)N(C23CC(C2)C3)CCN1Cc1ccc(-n2nccn2)nc1] | 1-((6-(2H-1,2,3-triazol-2- yl)pyridin-3-yl)methyl)-4- (bicyclo[1.1.1]pentan-1- yl)piperazine-2,3-dione | 339 |
| Example 117 | 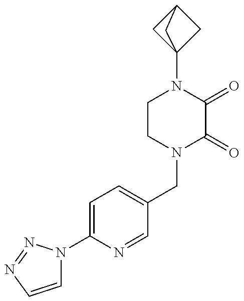 [SMILES: O=C1C(=O)N(C23CC(C2)C3)CCN1Cc1ccc(-n2ccnn2)nc1] | 1-((6-(1H-1,2,3-triazol-1- yl)pyridin-3-yl)methyl)-4- (bicyclo[1.1.1]pentan-1- yl)piperazine-2,3-dione | 339 |
| Example 118 | 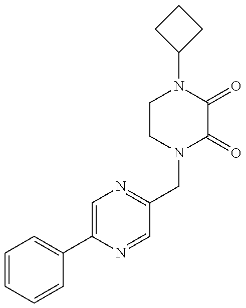 [SMILES: O=C1C(=O)N(C2CCC2)CCN1Cc1cnc(-c2ccccc2)cn1] | 1-cyclobutyl-4-((5- phenylpyrazin-2- yl)methyl)piperazine-2,3- dione | 337 |
| Example 119 | 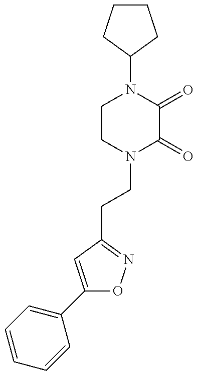 [SMILES: O=C1C(=O)N(C2CCCC2)CCN1CCc1cc(-c2ccccc2)on1] | 1-cyclopentyl-4-(2-(5- phenylisoxazol-3- yl)ethyl)piperazine-2,3-dione | 354 |
| Example 120 | 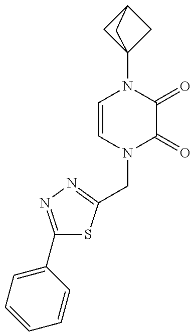 [SMILES: O=c1c(=O)n(C23CC(C2)C3)ccn1Cc1nnc(-c2ccccc2)s1] | 1-(bicyclo[1.1.1]pentan-1-yl)- 4-((5-phenyl-1,3,4-thiadiazol- 2-yl)methyl)-1,4- dihydropyrazine-2,3-dione | 353 |
| Example 121 | 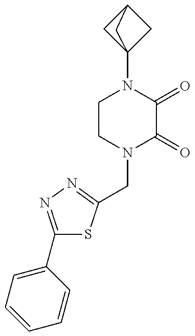 [SMILES: O=C1C(=O)N(C23CC(C2)C3)CCN1Cc1nnc(-c2ccccc2)s1] | 1-(bicyclo[1.1.1]pentan-1-yl)- 4-((5-phenyl-1,3,4-thiadiazol- 2-yl)methyl)piperazine-2,3- dione | 355 |
| Example 122 | 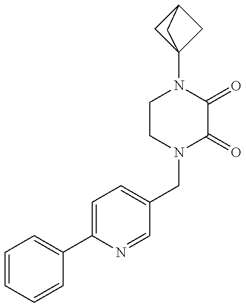 [SMILES: O=C1C(=O)N(C23CC(C2)C3)CCN1Cc1ccc(-c2ccccc2)nc1] | 1-(bicyclo[1.1.1]pentan-1-yl)- 4-((6-phenylpyridin-3- yl)methyl)piperazine-2,3- dione | 348 |
| Example 123 | 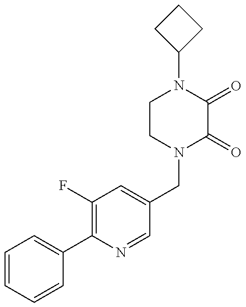 [SMILES: O=C1C(=O)N(C2CCC2)CCN1Cc1cnc(-c2ccccc2)c(F)c1] | 1-cyclobutyl-4-((5-fluoro-6- phenylpyridin-3- yl)methyl)piperazine-2,3- dione | 354 |
| Example 124 | 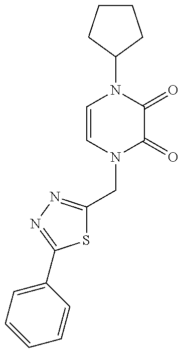 [SMILES: O=c1c(=O)n(C2CCCC2)ccn1Cc1nnc(-c2ccccc2)s1] | 1-cyclopentyl-4-((5-phenyl- 1,3,4-thiadiazol-2-yl)methyl)- 1,4-dihydropyrazine-2,3- dione | 355 |
| Example 125 | 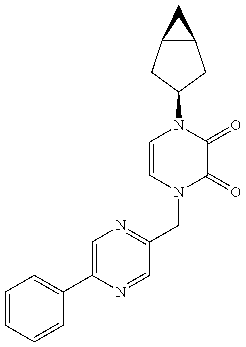 [SMILES: O=c1c(=O)n([C@H]2C[C@H]3C[C@H]3C2)ccn1Cc1cnc(-c2ccccc2)cn1] | 1-(cis)-bicyclo[3.1.0]hexan-3- yl)-4-((5-phenylpyrazin-2- yl)methyl)-1,4- dihydropyrazine-2,3-dione | 361 |
| Example 126 | 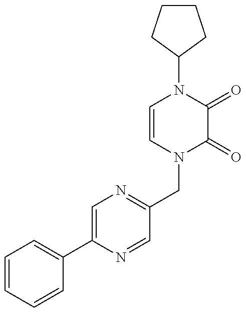 [SMILES: O=c1c(=O)n(C2CCCC2)ccn1Cc1cnc(-c2ccccc2)cn1] | 1-cyclopentyl-4-((5- phenylpyrazin-2-yl)methyl)- 1,4-dihydropyrazine-2,3- dione | 349 |
| Example 127 | 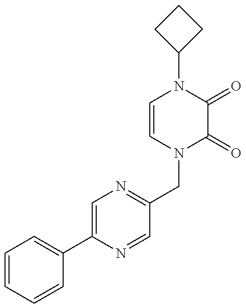 [SMILES: O=c1c(=O)n(C2CCC2)ccn1Cc1cnc(-c2ccccc2)cn1] | 1-cyclobutyl-4-((5- phenylpyrazin-2-yl)methyl)- 1,4-dihydropyrazine-2,3- dione | 335 |
| Example 128 | 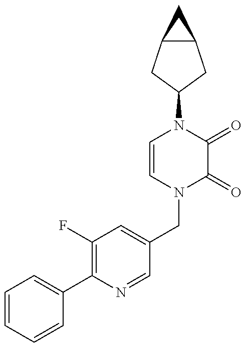 [SMILES: O=c1c(=O)n([C@H]2C[C@H]3C[C@H]3C2)ccn1Cc1cnc(-c2ccccc2)c(F)c1] | 1-((cis)-bicyclo[3.1.0]hexan- 3-yl)-4-((5-fluoro-6- phenylpyridin-3-yl)methyl)- 1,4-dihydropyrazine-2,3- dione | 378 |
| Example 129 | 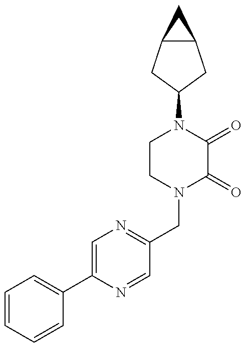 [SMILES: O=C1C(=O)N([C@H]2C[C@H]3C[C@H]3C2)CCN1Cc1cnc(-c2ccccc2)cn1] | 1-((cis)-bicyclo[3.1.0]hexan- 3-yl)-4-((5-phenylpyrazin-2- yl)methyl)piperazine-2,3- dione | 363 |
| Example 130 | 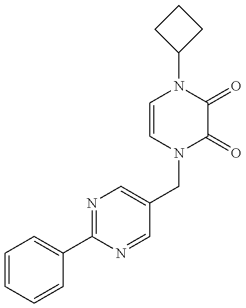 [SMILES: O=c1c(=O)n(C2CCC2)ccn1Cc1cnc(-c2ccccc2)nc1] | 1-cyclobutyl-4-((2- phenylpyrimidin-5- yl)methyl)-1,4- dihydropyrazine-2,3-dione | 335 |
| Example 131 | 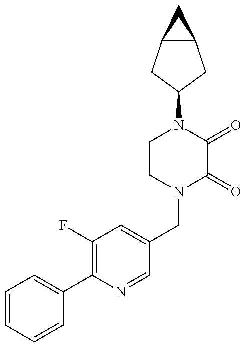 [SMILES: O=C1C(=O)N([C@H]2C[C@H]3C[C@H]3C2)CCN1Cc1cnc(-c2ccccc2)c(F)c1] | 1-((cis)-bicyclo[3.1.0]hexan- 3-yl)-4-((5-fluoro-6- phenylpyridin-3- yl)methyl)piperazine-2,3- dione | 380 |
| Example 132 | 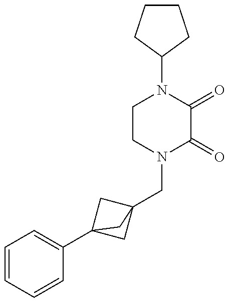 [SMILES: O=C1C(=O)N(C2CCCC2)CCN1CC12CC(c3ccccc3)(C1)C2] | 1-cyclopentyl-4-((3- phenylbicyclo[1.1.1]pentan- 1-yl)methyl)piperazine-2,3- dione | 339 |

### Example 133

### Preparation of (R or S′)-1-cyclopentyl-4-(1-(5-phenylpyrimidin-2-yl)ethyl)piperazine-2,3-dione

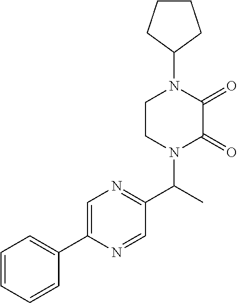 [SMILES: CC(c1cnc(-c2ccccc2)cn1)N1CCN(C2CCCC2)C(=O)C1=O]

Cs₂CO₃ (619 mg, 1.90 mmol) was added to a mixture of 1-cyclopentylpiperazine-2,3-dione (138 mg, 0.760 mmol) and 2-(1-bromoethyl)-5-phenylpyrimidine (0.200 g, 0.760 mmol) in DMF (2 mL) at room temperature. The reaction mixture was stirred and heated at 60° C. for 12 hours. The mixture was concentrated under reduced pressure. The residue was partitioned between water (10 mL) and DCM (10 mL). The organic layer was separated, and the aqueous layer was washed with additional DCM (3×10 mL). The organic layers were combined, washed with brine (10 mL), dried over anhydrous Na₂SO₄, filtered, and concentrated under reduced pressure. The residue was purified by silica gel chromatography (eluting ethyl acetate in petroleum ether) to afford a mixture of (R and S)-1-cyclopentyl-4-(1-(5-phenylpyrimidin-2-yl)ethyl)piperazine-2,3-dione. The racemic mixture was resolved by chiral-SFC (Phenomenex Cellulose-2 (250 mm×30 mm, 10 uM) 45% methanol with 0.1% ammonia as eluent) to afford (R or S)-1-cyclopentyl-4-(1-(5-phenylpyrimidin-2-yl)ethyl)piperazine-2,3-dione as the second eluting peak. ¹H NMR (500 MHz, methanol-d₄) δ=9.03 (s, 2H), 7.71 (d, J=7.3 Hz, 2H), 7.56-7.51 (m, 2H), 7.50-7.45 (m, 1H), 5.82 (q, J=7.3 Hz, 1H), 4.84-4.77 (m, 1H), 3.71-3.66 (m, 3H), 3.61-3.55 (m, 1H), 1.97-1.87 (m, 2H), 1.79 (br s, 2H), 1.72 (d, J=7.3 Hz, 3H), 1.69-1.62 (m, 4H). LC/MS (m/z): 365 (M+H)⁺

### Example 134

### Preparation of (R or S)-1-cyclobutyl-4-(1-(5-phenylpyrimidin-2-yl)ethyl)piperazine-2,3-dione

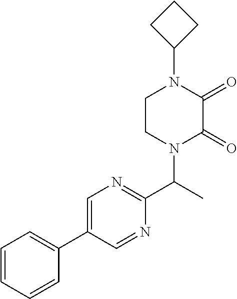 [SMILES: CC(c1ncc(-c2ccccc2)cn1)N1CCN(C2CCC2)C(=O)C1=O]

Cs₂CO₃ (226 mg, 0.694 mmol) was added to a mixture of 1-cyclobutylpiperazine-2,3-dione (47 mg, 0.28 mmol) and 2-(1-bromoethyl)-5-phenylpyrimidine (73 mg, 0.28 mmol) in DMF (3 mL) at room temperature. The mixture was stirred and heated at 60° C. for 1 hour. The reaction was cooled to room temperature and concentrated under reduced pressure. The residue was partitioned between water (10 mL) and DCM (10 mL). The organic layer was separated, and the aqueous was washed with additional DCM (3×10 mL). The organic layers were combined, washed with brine (10 mL), dried over anhydrous Na₂SO₄, filtered, and concentrated under reduced pressure. The residue was purified by silica gel chromatography (eluting methanol in dichloromethane) to afford a mixture of (R and S)-1-cyclobutyl-4-(1-(5-phenylpyrimidin-2-yl)ethyl)piperazine-2,3-dione. The racemic mixture was resolved by chiral-SFC (Phenomenex Cellulose-2 (250 mm×30 mm, 10 uM) 45% methanol with 0.1% ammonia as eluent) to afford (R or S)-1-cyclobutyl-4-(1-(5-phenylpyrimidin-2-yl)ethyl)piperazine-2,3-dione as the second eluting peak. ¹H NMR (400 MHz, methanol-d₄) δ 9.03 (s, 2H), 7.72-7.69 (m, 2H), 7.56-7.51 (m, 1H), 7.50-7.45 (m, 1H), 5.89-5.75 (m, 1H), 4.77 (s, 1H), 3.78-3.64 (m, 4H), 2.32-2.16 (m, 4H), 1.84-1.74 (m, 2H), 1.74-1.70 (m, 3H). LC/MS (m/z): 351 (M+H)⁺

### Example 135

### Preparation of 1-cyclobutyl-4-((5-phenyl-1,3,4-thiadiazol-2-yl)methyl)piperazine-2,3-dione

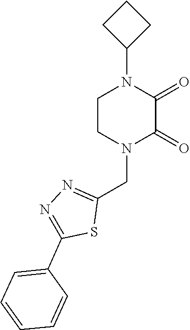 [SMILES: O=C1C(=O)N(C2CCC2)CCN1Cc1nnc(-c2ccccc2)s1]

A mixture of 1-cyclobutylpiperazine-2,3-dione (1.50 g, 8.92 mmol), 2-(chloromethyl)-5-phenyl-1,3,4-thiadiazole (1.88 g, 8.92 mmol), and Cs₂CO₃ (3.49 g, 10.7 mmol) in DMF (20 mL) was stirred and heated at 60° C. for 2 hours. The mixture was cooled to room temperature, filtered, and concentrated under reduced pressure. The residue was purified by silica gel chromatography (eluting methanol in dichloromethane) to afford 1-cyclobutyl-4-((5-phenyl-1,3,4-thiadiazol-2-yl)methyl)piperazine-2,3-dione. ¹H NMR (500 MHz, DMSO-d₆) δ 7.97 (dd, J=7.6 Hz, 1.7 Hz, 2H), 7.65-7.50 (m, 3H), 5.03 (s, 2H), 4.80-4.70 (m, 1H), 3.73-3.68 (m, 2H), 3.66-3.57 (m, 2H), 2.25-2.11 (m, 2H), 2.10-2.00 (m, 2H), 1.73-1.60 (m, 2H). LC/MS (m/z): 343 (M+H)⁺

### Example 136

### Preparation of 1-((5-(2H-1,2,3-triazol-2-yl)pyridin-2-yl)methyl)-4-cyclobutylpiperazine-2,3-dione

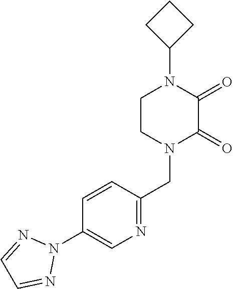 [SMILES: O=C1C(=O)N(C2CCC2)CCN1Cc1ccc(-n2nccn2)cn1]

A mixture of 1-cyclobutylpiperazine-2,3-dione (8.56 g, 50.9 mmol), 2-(chloromethyl)-5-(2H-1,2,3-triazol-2-yl)pyridine (9.00 g, 46.2 mmol), and potassium carbonate (12.8 g, 92.0 mmol) in acetonitrile (100 mL) was heated to 65° C. and stirred for 3 days. The reaction mixture was cooled to room temperature and diluted with 10:1 DCM/methanol (500 mL). The mixture was stirred for 15 minutes and then filtered (to remove potassium salts). Silica gel (80 g) was added directly to the filtrate, and the filtrate was then concentrated under reduced pressure to afford the crude product adsorbed onto silica. The silica-adsorbed product was purified by silica gel chromatography (eluting methanol in dichloromethane) to afford the product as solids. The solids were suspended in 20% methanol in ethyl acetate (900 mL) at room temperature. The mixture was stirred and heated to 70° C. until all solids were dissolved (˜1.5 hrs). The mixture was then concentrated under reduced pressure. The isolated residue was then dried under vacuum. The solids were suspended in ethyl acetate (250 mL) at room temperature. Hexanes (50 mL) were added dropwise to the stirred mixture over a period of 1 hour. The suspension was stirred at room temperature for 18 hours. The suspension was filtered, and the collected solids were washed with 1:1 ethyl acetate/hexanes (40 mL). The collected solids were dried under vacuum to afford 1-((5-(2H-1,2,3-triazol-2-yl)pyridin-2-yl)methyl)-4-cyclobutylpiperazine-2,3-dione. ¹H NMR (500 MHz, DMSO-d₆) δ 9.19 (d, J=2.3 Hz, 1H), 8.37 (dd, J=8.5, 2.6 Hz, 1H), 8.21 (s, 2H), 7.59 (d, J=8.5 Hz, 1H), 4.82-4.73 (m, 1H), 4.76 (s, 2H), 3.63 (s, 4H), 2.24-2.14 (m, 2H), 2.10-2.03 (m, 2H), 1.70-1.62 (m, 2H). LC/MS (m/z): 327 (M+H)⁺

### Example 137

### Preparation of 1-cyclobutyl-4-((6-phenylpyridazin-3-yl)methyl)-1,4-dihydropyrazine-2,3-dione

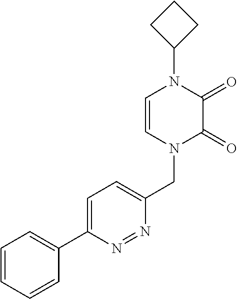 [SMILES: O=c1c(=O)n(C2CCC2)ccn1Cc1ccc(-c2ccccc2)nn1]

A mixture of 1-cyclobutyl-1,4-dihydropyrazine-2,3-dione (2.44 g, 14.7 mmol), 3-(chloromethyl)-6-phenylpyridazine (3.00 g, 14.7 mmol), and Cs₂CO₃ (4.78 g, 14.7 mmol) in DMF (20 mL) was stirred and heated at 60° C. for 1 hour. The mixture was cooled to room temperature, filtered, and concentrated under reduced pressure. The residue was purified by silica gel chromatography (eluting ethyl acetate in dichloromethane) to afford 1-cyclobutyl-4-((6-phenylpyridazin-3-yl)methyl)-1,4-dihydropyrazine-2,3-dione. ¹H NMR (500 MHz, chloroform-d) δ 8.10-8.03 (m, 2H), 7.92-7.84 (m, 1H), 7.82-7.76 (m, 1H), 7.58-7.47 (m, 3H), 6.61 (d, J=6.3 Hz, 1H), 6.38 (d, J=6.3 Hz, 1H), 5.28 (s, 2H), 5.10-4.92 (m, 1H), 2.41-2.38 (m, 2H), 2.17-2.14 (m, 2H), 1.89-1.74 (m, 2H). LC/MS (m/z): 335 (M+H)⁺

### Example 138

### Preparation of 1-cyclobutyl-4-((5-(pyridin-2-yl)isoxazol-3-yl)methyl)piperazine-2,3-dione

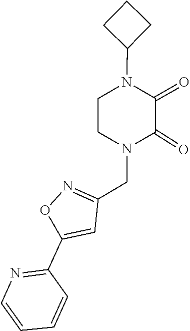 [SMILES: O=C1C(=O)N(C2CCC2)CCN1Cc1cc(-c2ccccn2)on1]

A mixture of 1-cyclobutylpiperazine-2,3-dione (1.99 g, 11.8 mmol), (5-(pyridin-2-yl)isoxazol-3-yl)methyl methanesulfonate (3.00 g, 11.8 mmol), and Cs₂CO₃ (5.00 g, 15.3 mmol) in DMF (25 mL) was stirred and heated at 60° C. for 2 hours. The reaction mixture was cooled to room temperature and filtered. The filtrate was concentrated under reduced pressure, and the residue was purified by silica gel chromatography (eluting methanol in dichloromethane) followed by purification by reverse phase HPLC (eluting acetonitrile in water, NH₄HCO₃ modifier) to afford 1-cyclobutyl-4-((5-(pyridin-2-yl)isoxazol-3-yl)methyl)piperazine-2,3-dione. ¹H NMR (400 MHz, chloroform-d) δ 8.79-8.48 (m, 1H), 7.97-7.73 (m, 2H), 7.40-7.31 (m, 1H), 6.93 (s, 1H), 4.99 (quin, J=8.8 Hz, 1H), 4.81 (s, 2H), 3.65-3.58 (m, 2H), 3.58-3.52 (m, 2H), 2.25-2.15 (m, 2H), 2.12-2.00 (m, 2H), 1.80-1.71 (m, 2H). LC/MS (m/z): 327 (M+H)⁺

### Example 139

### Preparation of 1-cyclopentyl-4-((3-fluoro-5-(thiazol-2-yl)pyridin-2-yl)methyl)piperazine-2,3-dione

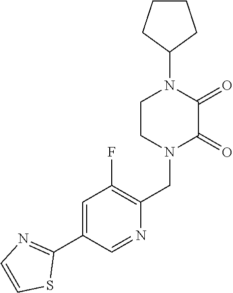 [SMILES: O=C1C(=O)N(C2CCCC2)CCN1Cc1ncc(-c2nccs2)cc1F]

A mixture of (3-fluoro-5-(thiazol-2-yl)pyridin-2-yl)methyl methanesulfonate (5 mg, 0.02 mmol), 1-cyclopentylpiperazine-2,3-dione (4 mg, 0.02 mmol), and Cs₂CO₃ (10 mg, 0.03 mmol) in DMF (0.5 mL) was stirred and heated at 80° C. for 1 hour. The reaction mixture was cooled to room temperature, diluted with DCM (3 mL), and filtered through Celite. The filtrate was concentrated under reduced pressure, and the residue was purified by basic alumina chromatography (eluting [EtOAc:EtOH (3:1)] in hexanes) to afford 1-cyclopentyl-4-((3-fluoro-5-(thiazol-2-yl)pyridin-2-yl)methyl)piperazine-2,3-dione. ¹H NMR (500 MHz, DMSO-d₆) δ 8.98 (s, 1H), 8.25 (d, J=10.5 Hz, 1H), 8.04 (d, J=3.2 Hz, 1H), 7.95 (d, J=3.2 Hz, 1H), 4.85 (s, 2H), 4.75-4.65 (m, 1H), 3.67-3.60 (m, 2H), 3.58-3.49 (m, 2H), 1.82-1.74 (m, 2H), 1.70-1.64 (m, 2H), 1.60-1.52 (m, 4H). LC/MS (m/z): 375 (M+H)⁺

Examples shown in Example Table 3 below, were or may be prepared according to procedures analogous to those outlined in Example 139 above using the appropriate starting materials, described in the Preparations or Intermediates above, or as obtained from commercial

EXAMPLE TABLE 3

| Example | Structure | Name | Mass [M + H]⁺ |
|---|---|---|---|
| Example 140 | 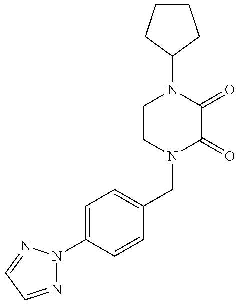 [SMILES: O=C1C(=O)N(C2CCCC2)CCN1Cc1ccc(-n2nccn2)cc1] | 1-(4-(2H-1,2,3-triazol-2- yl)benzyl)-4- cyclopentylpiperazine-2,3-dione | 340 |
| Example 141 | 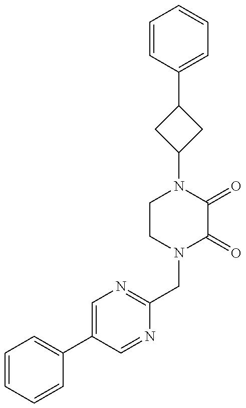 [SMILES: O=C1C(=O)N(C2CC(c3ccccc3)C2)CCN1Cc1ncc(-c2ccccc2)cn1] | 1-(3-phenylcyclobutyl)-4-((5- phenylpyrimidin-2- yl)methyl)piperazine-2,3-dione | 413 |
| Example 142 | 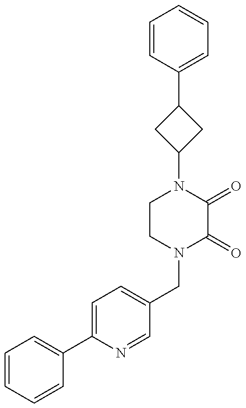 [SMILES: O=C1C(=O)N(C2CC(c3ccccc3)C2)CCN1Cc1ccc(-c2ccccc2)nc1] | 1-(3-phenylcyclobutyl)-4-((6- phenylpyridin-3- yl)methyl)piperazine-2,3- dione | 412 |
| Example 143 | 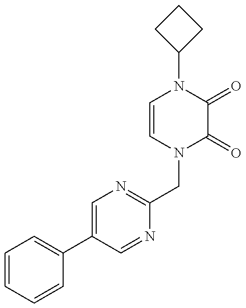 [SMILES: O=c1c(=O)n(C2CCC2)ccn1Cc1ncc(-c2ccccc2)cn1] | 1-cyclobutyl-4-((5- phenylpyrimidin-2-yl)methyl)- 1,4-dihydropyrazine-2,3-dione | 335 |
| Example 144 | 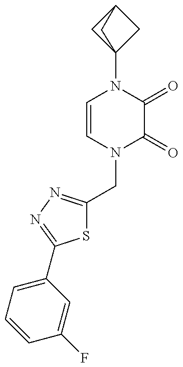 [SMILES: O=c1c(=O)n(C23CC(C2)C3)ccn1Cc1nnc(-c2cccc(F)c2)s1] | 1-(bicyclo[1.1.1]pentan-1-yl)-4- ((5-(3-fluorophenyl)-1,3,4- thiadiazol-2-yl)methyl)-1,4- dihydropyrazine-2,3-dione | 371 |
| Example 145 | 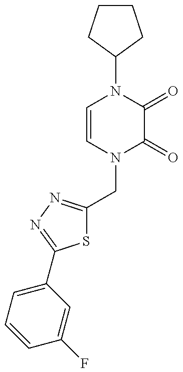 [SMILES: O=c1c(=O)n(C2CCCC2)ccn1Cc1nnc(-c2cccc(F)c2)s1] | 1-cyclopentyl-4-((5-(3- fluorophenyl)-1,3,4-thiadiazol-2- yl)methyl)-1,4-dihydropyrazine- 2,3-dione | 373 |
| Example 146 | 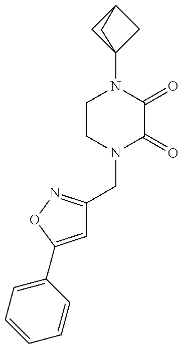 [SMILES: O=C1C(=O)N(C23CC(C2)C3)CCN1Cc1cc(-c2ccccc2)on1] | 1-(bicyclo[1.1.1]pentan-1-yl)-4- ((5-phenylisoxazol-3- yl)methyl)piperazine-2,3-dione | 338 |
| Example 147 | 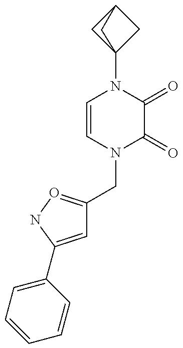 | 1-(bicyclo[1.1.1]pentan-1-yl)-4- ((5-phenylisoxazol-3-yl)methyl)- 1,4-dihydropyrazine-2,3-dione | 336 |
| Example 148 | 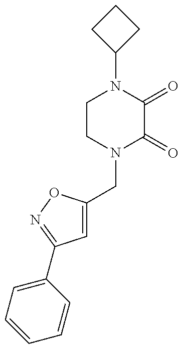 [SMILES: O=C1C(=O)N(C2CCC2)CCN1Cc1cc(-c2ccccc2)no1] | 1-cyclobutyl-4-((3- phenylisoxazol-5- yl)methyl)piperazine-2,3-dione | 326 |
| Example 149 | 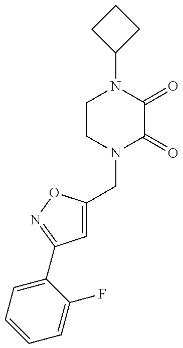 [SMILES: O=C1C(=O)N(C2CCC2)CCN1Cc1cc(-c2ccccc2F)no1] | 1-cyclobutyl-4-((3-(2- fluorophenyl)isoxazol-5- yl)methyl)piperazine-2,3-dione | 344 |
| Example 150 | 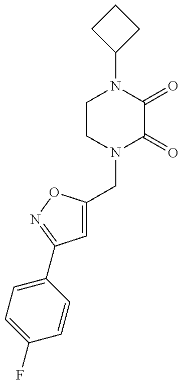 [SMILES: O=C1C(=O)N(C2CCC2)CCN1Cc1cc(-c2ccc(F)cc2)no1] | 1-cyclobutyl-4-((3-(4- fluorophenyl)isoxazol-5- yl)methyl)piperazine-2,3-dione | 344 |
| Example 151 | 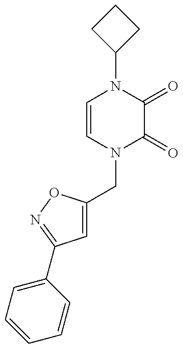 [SMILES: O=c1c(=O)n(C2CCC2)ccn1Cc1cc(-c2ccccc2)no1] | 1-cyclobutyl-4-((3- phenylisoxazol-5-yl)methyl)- 1,4-dihydropyrazine-2,3-dione | 324 |
| Example 152 | 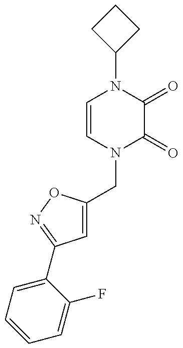 [SMILES: O=c1c(=O)n(C2CCC2)ccn1Cc1cc(-c2ccccc2F)no1] | 1-cyclobutyl-4-((3-(2- fluorophenyl)isoxazol-5- yl)methyl)-1,4-dihydropyrazine- 2,3-dione | 342 |
| Example 153 | 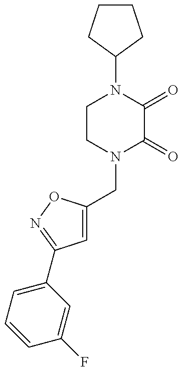 [SMILES: O=C1C(=O)N(C2CCCC2)CCN1Cc1cc(-c2cccc(F)c2)no1] | 1-cyclopentyl-4-((3-(3- fluorophenyl)isoxazol-5- yl)methyl)piperazine-2,3-dione | 358 |
| Example 154 | 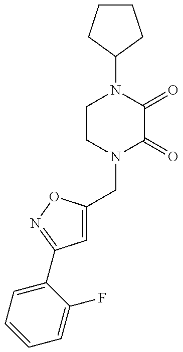 [SMILES: O=C1C(=O)N(C2CCCC2)CCN1Cc1cc(-c2ccccc2F)no1] | 1-cyclopentyl-4-((3-(2- fluorophenyl)isoxazol-5- yl)methyl)piperazine-2,3-dione | 358 |
| Example 155 | 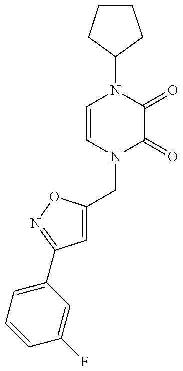 [SMILES: O=c1c(=O)n(C2CCCC2)ccn1Cc1cc(-c2cccc(F)c2)no1] | 1-cyclopentyl-4-((3-(3- fluorophenyl)isoxazol-5- yl)methyl)-1,4-dihydropyrazine- 2,3-dione | 356 |
| Example 156 | 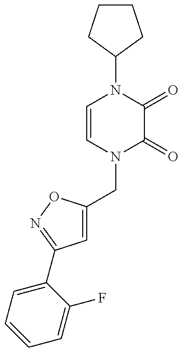 [SMILES: O=c1c(=O)n(C2CCCC2)ccn1Cc1cc(-c2ccccc2F)no1] | 1-cyclopentyl-4-((3-(2- fluorophenyl)isoxazol-5- yl)methyl)-1,4-dihydropyrazine- 2,3-dione | 356 |
| Example 157 | 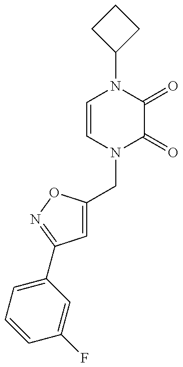 [SMILES: O=c1c(=O)n(C2CCC2)ccn1Cc1cc(-c2cccc(F)c2)no1] | 1-cyclobutyl-4-((3-(3- fluorophenyl)isoxazol-5- yl)methyl)-1,4-dihydropyrazine- 2,3-dione | 342 |
| Example 158 | 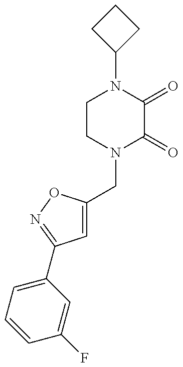 [SMILES: O=C1C(=O)N(C2CCC2)CCN1Cc1cc(-c2cccc(F)c2)no1] | 1-cyclobutyl-4-((3-(3- fluorophenyl)isoxazol-5- yl)methyl)piperazine-2,3-dione | 344 |
| Example 159 | 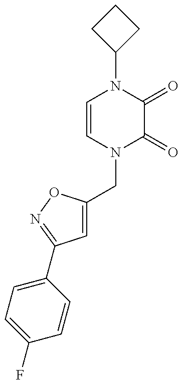 [SMILES: O=c1c(=O)n(C2CCC2)ccn1Cc1cc(-c2ccc(F)cc2)no1] | 1-cyclobutyl-4-((3-(4- fluorophenyl)isoxazol-5- yl)methyl)-1,4-dihydropyrazine- 2,3-dione | 342 |
| Example 160 | 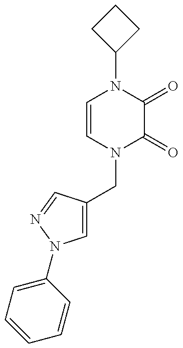 [SMILES: O=c1c(=O)n(C2CCC2)ccn1Cc1cnn(-c2ccccc2)c1] | 1-cyclobutyl-4-((1-phenyl-1H- pyrazol-4-yl)methyl)-1,4- dihydropyrazine-2,3-dione | 323 |
| Example 161 | 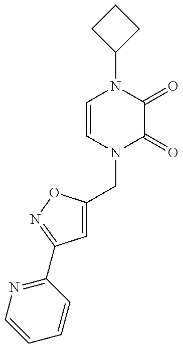 [SMILES: O=c1c(=O)n(C2CCC2)ccn1Cc1cc(-c2ccccn2)no1] | 1-cyclobutyl-4-((3-(pyridin-2- yl)isoxazol-5-yl)methyl)-1,4- dihydropyrazine-2,3-dione | 325 |
| Example 162 | 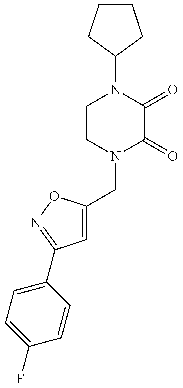 [SMILES: O=C1C(=O)N(C2CCCC2)CCN1Cc1cc(-c2ccc(F)cc2)no1] | 1-cyclopentyl-4-((3-(4- fluorophenyl)isoxazol-5- yl)methyl)piperazine-2,3-dione | 358 |
| Example 163 | 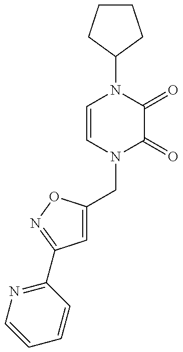 [SMILES: O=c1c(=O)n(C2CCCC2)ccn1Cc1cc(-c2ccccn2)no1] | 1-cyclopentyl-4-((3-(pyridin-2- yl)isoxazol-5-yl)methyl)-1,4- dihydropyrazine-2,3-dione | 339 |
| Example 164 | 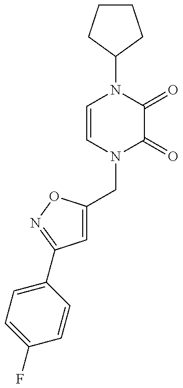 [SMILES: O=c1c(=O)n(C2CCCC2)ccn1Cc1cc(-c2ccc(F)cc2)no1] | 1-cyclopentyl-4-((3-(4- fluorophenyl)isoxazol-5- yl)methyl)-1,4-dihydropyrazine- 2,3-dione | 356 |
| Example 165 | 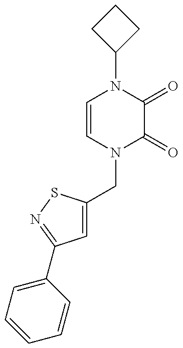 [SMILES: O=c1c(=O)n(C2CCC2)ccn1Cc1cc(-c2ccccc2)ns1] | 1-cyclobutyl-4-((3- phenylisoxazol-5-yl)methyl)- 1,4-dihydropyrazine-2,3-dione | 340 |
| Example 166 | 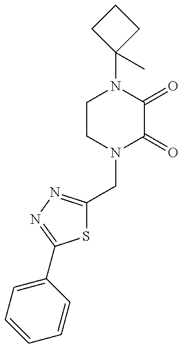 [SMILES: CC1(N2CCN(Cc3nnc(-c4ccccc4)s3)C(=O)C2=O)CCC1] | 1-(1-methylcyclobutyl)-4-((5- phenyl-1,3,4-thiadiazol-2- yl)methyl)piperazine-2,3-dione | 357 |
| Example 167 | 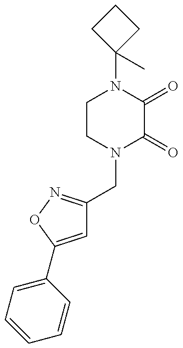 [SMILES: CC1(N2CCN(Cc3cc(-c4ccccc4)on3)C(=O)C2=O)CCC1] | 1-(1-methylcyclobutyl)-4-((5- phenylisoxazol-3- yl)methyl)piperazine-2,3-dione | 340 |
| Example 168 | 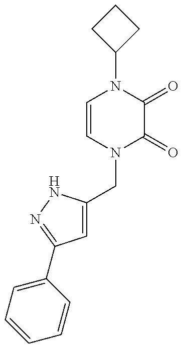 [SMILES: O=c1c(=O)n(C2CCC2)ccn1Cc1cc(-c2ccccc2)n[nH]1] | 1-cyclobutyl-4-((3-phenyl-1H- pyrazol-5-yl)methyl)-1,4- dihydropyrazine-2,3-dione | 323 |
| Example 169 | 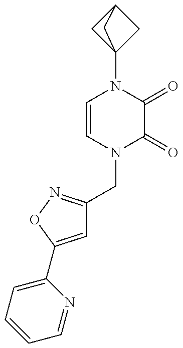 [SMILES: O=c1c(=O)n(C23CC(C2)C3)ccn1Cc1cc(-c2ccccn2)on1] | 1-(bicyclo[1.1.1]pentan-1-yl)-4- ((5-(pyridin-2-yl)isoxazol-3- yl)methyl)-1,4-dihydropyrazine- 2,3-dione | 337 |
| Example 170 | 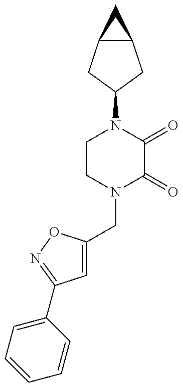 [SMILES: O=C1C(=O)N([C@H]2C[C@H]3C[C@H]3C2)CCN1Cc1cc(-c2ccccc2)no1] | 1-((cis)-bicyclo[3.1.0]hexan-3- yl)-4-((3-phenylisoxazol-5- yl)methyl)piperazine-2,3-dione | 352 |
| Example 171 | 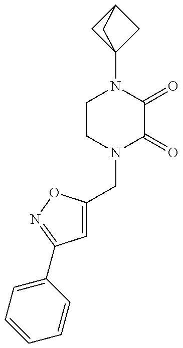 [SMILES: O=C1C(=O)N(C23CC(C2)C3)CCN1Cc1cc(-c2ccccc2)no1] | 1-(bicyclo[1.1.1]pentan-1-yl)-4- ((3-phenylisoxazol-5- yl)methyl)piperazine-2,3-dione | 338 |
| Example 172 | 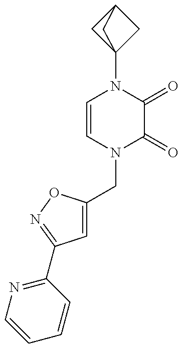 [SMILES: O=c1c(=O)n(C23CC(C2)C3)ccn1Cc1cc(-c2ccccn2)no1] | 1-(bicyclo[1.1.1]pentan-1-yl)-4- ((3-(pyridin-2-yl)isoxazol-5- yl)methyl)-1,4-dihydropyrazine- 2,3-dione | 337 |
| Example 173 | 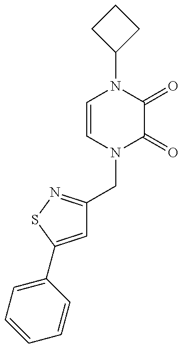 [SMILES: O=c1c(=O)n(C2CCC2)ccn1Cc1cc(-c2ccccc2)sn1] | 1-cyclobutyl-4-((5- phenylisothiazol-3-yl)methyl)- 1,4-dihydropyrazine-2,3-dione | 340 |
| Example 174 | 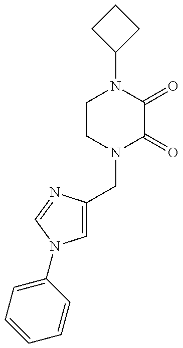 [SMILES: O=C1C(=O)N(C2CCC2)CCN1Cc1cn(-c2ccccc2)cn1] | 1-cyclobutyl-4-((1-phenyl-1H- imidazol-4- yl)methyl)piperazine-2,3-dione | 325 |
| Example 175 | 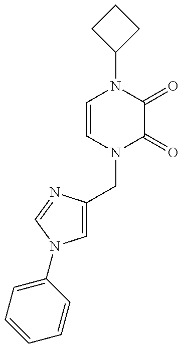 [SMILES: O=c1c(=O)n(C2CCC2)ccn1Cc1cn(-c2ccccc2)cn1] | 1-cyclobutyl-4-((1-phenyl-1H- imidazol-4-yl)methyl)-1,4- dihydropyrazine-2,3-dione | 323 |
| Example 176 | 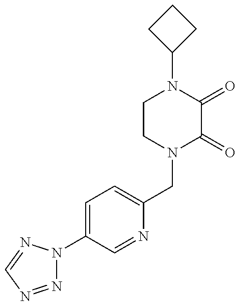 [SMILES: O=C1C(=O)N(C2CCC2)CCN1Cc1ccc(-n2ncnn2)cn1] | 1-((5-(2H-tetrazol-2-yl)pyridin- 2-yl)methyl)-4- cyclobutylpiperazine-2,3-dione | 328 |
| Example 177 | 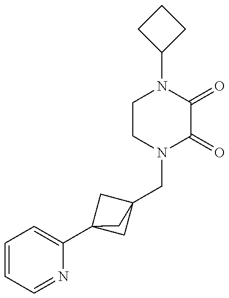 [SMILES: O=C1C(=O)N(C2CCC2)CCN1CC12CC(c3ccccn3)(C1)C2] | 1-cyclobutyl-4-((3-(pyridin-2- yl)bicyclo[1.1.1]pentan-1- yl)methyl)piperazine-2,3-dione | 326 |
| Example 178 | 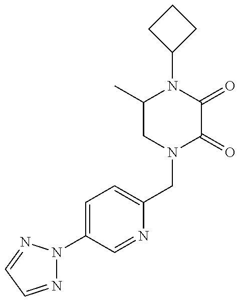 [SMILES: CC1CN(Cc2ccc(-n3nccn3)cn2)C(=O)C(=O)N1C1CCC1] | 1-((5-(2H-1,2,3-triazol-2- yl)pyridin-2-yl)methyl)-4- cyclobutyl-5-methylpiperazine- 2,3-dione | 341 |
| Example 179 | 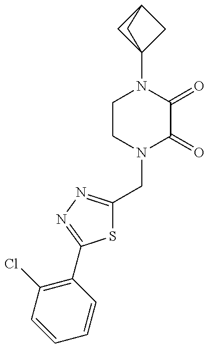 [SMILES: O=C1C(=O)N(C23CC(C2)C3)CCN1Cc1nnc(-c2ccccc2Cl)s1] | 1-(bicyclo[1.1.1]pentan-1-yl)-4- ((5-(2-chlorophenyl)-1,3,4- thiadiazol-2- yl)methyl)piperazine-2,3-dione | 389 |
| Example 180 | 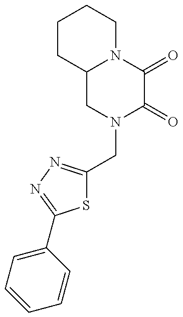 [SMILES: O=C1C(=O)N2CCCCC2CN1Cc1nnc(-c2ccccc2)s1] | (R or S)-2-((5-phenyl-1,3,4- thiadiazol-2- yl)methyl)hexahydro-2H- pyrido[1,2-a]pyrazine-3,4-dione | 343 |
| Example 181 | 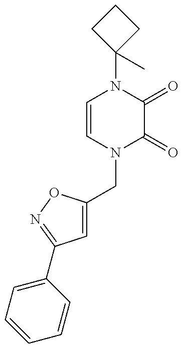 [SMILES: CC1(n2ccn(Cc3cc(-c4ccccc4)no3)c(=O)c2=O)CCC1] | 1-(1-methylcyclobutyl)-4-((3- phenylisoxazol-5-yl)methyl)- 1,4-dihydropyrazine-2,3-dione | 338 |
| Example 182 | 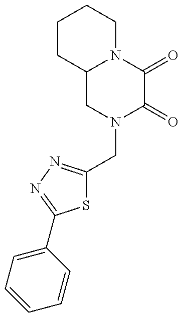 [SMILES: O=C1C(=O)N2CCCCC2CN1Cc1nnc(-c2ccccc2)s1] | (R or S)-2-((5-phenyl-1,3,4- thiadiazol-2- yl)methyl)hexahydro-2H- pyrido[1,2-a]pyrazine-3,4-dione | 343 |
| Example 183 | 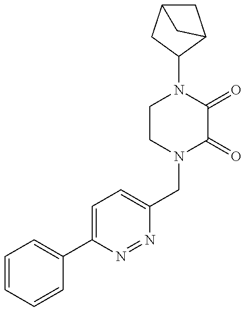 [SMILES: O=C1C(=O)N(C2CC3CC2C3)CCN1Cc1ccc(-c2ccccc2)nn1] | 1-(bicyclo[2.1.1]hexan-2-yl)-4- ((6-phenylpyridazin-3- yl)methyl)piperazine-2,3-dione | 363 |
| Example 184 | 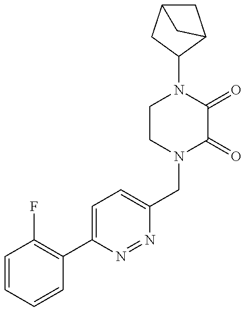 [SMILES: O=C1C(=O)N(C2CC3CC2C3)CCN1Cc1ccc(-c2ccccc2F)nn1] | 1-(bicyclo[2.1.1]hexan-2-yl)-4- ((6-(2-fluorophenyl)pyridazin-3- yl)methyl)piperazine-2,3-dione | 381 |
| Example 185 | 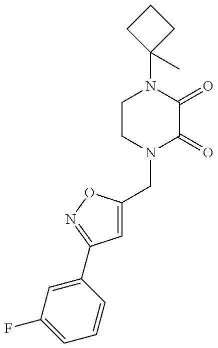 [SMILES: CC1(N2CCN(Cc3cc(-c4cccc(F)c4)no3)C(=O)C2=O)CCC1] | 1-((3-(3-fluorophenyl)isoxazol- 5-yl)methyl)-4-(1- methylcyclobutyl)piperazine- 2,3-dione | 358 |
| Example 186 | 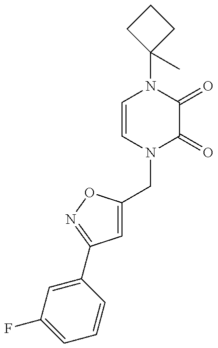 [SMILES: CC1(n2ccn(Cc3cc(-c4cccc(F)c4)no3)c(=O)c2=O)CCC1] | 1-((3-(3-fluorophenyl)isoxazol- 5-yl)methyl)-4-(1- methylcyclobutyl)-1,4- dihydropyrazine-2,3-dione | 356 |
| Example 187 | 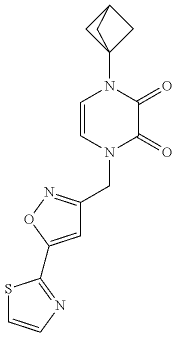 [SMILES: O=c1c(=O)n(C23CC(C2)C3)ccn1Cc1cc(-c2nccs2)on1] | 1-(bicyclo[1.1.1]pentan-1-yl)-4- ((5-(thiazol-2-yl)isoxazol-3- yl)methyl)-1,4-dihydropyrazine- 2,3-dione | 343 |
| Example 188 | 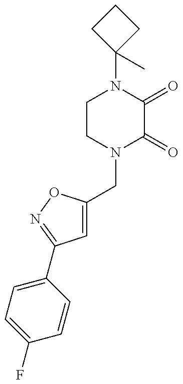 [SMILES: CC1(N2CCN(Cc3cc(-c4ccc(F)cc4)no3)C(=O)C2=O)CCC1] | 1-((3-(4-fluorophenyl)isoxazol- 5-yl)methyl)-4-(1- methylcyclobutyl)piperazine- 2,3-dione | 358 |
| Example 189 | 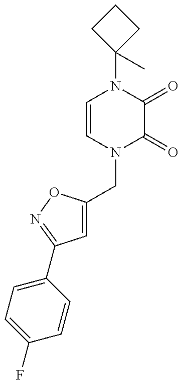 [SMILES: CC1(n2ccn(Cc3cc(-c4ccc(F)cc4)no3)c(=O)c2=O)CCC1] | 1-((3-(4-fluorophenyl)isoxazol- 5-yl)methyl)-4-(1- methylcyclobutyl)-1,4- dihydropyrazine-2,3-dione | 356 |
| Example 190 | 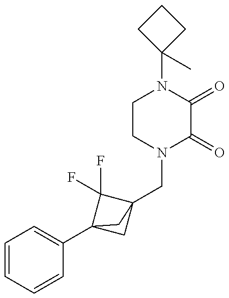 [SMILES: CC1(N2CCN(CC34CC(c5ccccc5)(C3)C4(F)F)C(=O)C2=O)CCC1] | 1-((2,2-difluoro-3- phenylbicyclo[1.1.1]pentan-1- yl)methyl)-4-(1- methylcyclobutyl)piperazine- 2,3-dione | 375 |
| Example 191 | 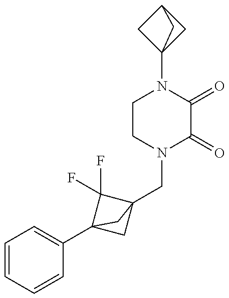 [SMILES: O=C1C(=O)N(C23CC(C2)C3)CCN1CC12CC(c3ccccc3)(C1)C2(F)F] | 1-(bicyclo[1.1.1]pentan-1-yl)-4- ((2,2-difluoro-3- phenylbicyclo[1.1.1]pentan-1- yl)methyl)piperazine-2,3-dione | 373 |
| Example 192 | 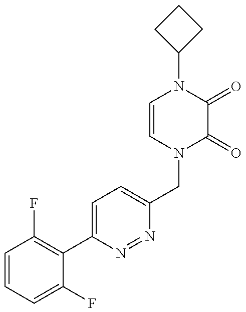 [SMILES: O=c1c(=O)n(C2CCC2)ccn1Cc1ccc(-c2c(F)cccc2F)nn1] | 1-cyclobutyl-4-((6-(2,6- difluorophenyl)pyridazin-3- yl)methyl)-1,4-dihydropyrazine- 2,3-dione | 371 |
| Example 193 | 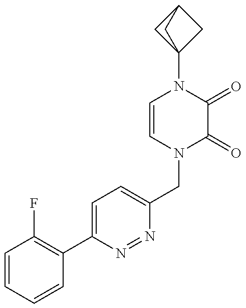 [SMILES: O=c1c(=O)n(C23CC(C2)C3)ccn1Cc1ccc(-c2ccccc2F)nn1] | 1-(bicyclo[1.1.1]pentan-1-yl)-4- ((6-(2-fluorophenyl)pyridazin-3- yl)methyl)-1,4-dihydropyrazine- 2,3-dione | 365 |
| Example 194 | 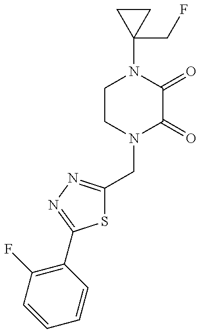 [SMILES: O=C1C(=O)N(C2(CF)CC2)CCN1Cc1nnc(-c2ccccc2F)s1] | 1-(1-(fluoromethyl)cyclopropyl)- 4-((5-(2-fluorophenyl)-1,3,4- thiadiazol-2- yl)methyl)piperazine-2,3-dione | 379 |
| Example 195 | 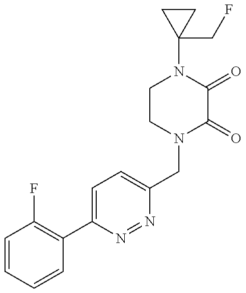 [SMILES: O=C1C(=O)N(C2(CF)CC2)CCN1Cc1ccc(-c2ccccc2F)nn1] | 1-(1-(fluoromethyl)cyclopropyl)- 4-((6-(2-fluorophenyl)pyridazin- 3-yl)methyl)piperazine-2,3- dione | 373 |
| Example 196 | 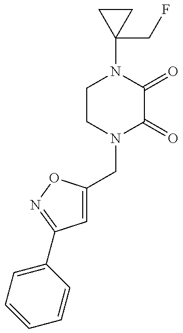 [SMILES: O=C1C(=O)N(C2(CF)CC2)CCN1Cc1cc(-c2ccccc2)no1] | 1-(1-(fluoromethyl)cyclopropyl)- 4-((3-phenylisoxazol-5- yl)methyl)piperazine-2,3-dione | 344 |
| Example 197 | 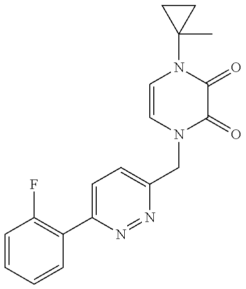 [SMILES: CC1(n2ccn(Cc3ccc(-c4ccccc4F)nn3)c(=O)c2=O)CC1] | 1-((6-(2-fluorophenyl)pyridazin- 3-yl)methyl)-4-(1- methylcyclopropyl)-1,4- dihydropyrazine-2,3-dione | 353 |
| Example 198 | 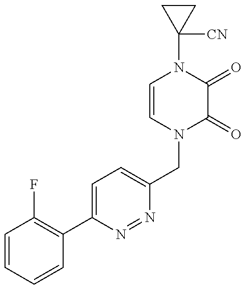 [SMILES: N#CC1(n2ccn(Cc3ccc(-c4ccccc4F)nn3)c(=O)c2=O)CC1] | 1-(4-((6-(2- fluorophenyl)pyridazin-3- yl)methyl)-2,3-dioxo-3,4- dihydropyrazin-1(2H)- yl)cyclopropane-1-carbonitrile | 364 |
| Example 199 | 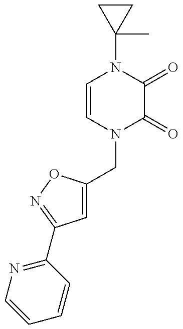 [SMILES: CC1(n2ccn(Cc3cc(-c4ccccn4)no3)c(=O)c2=O)CC1] | 1-(1-methylcyclopropyl)-4-((3- (pyridin-2-yl)isoxazol-5- yl)methyl)-1,4-dihydropyrazine- 2,3-dione | 325 |
| Example 200 | 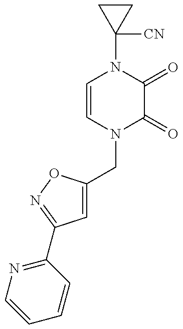 [SMILES: N#CC1(n2ccn(Cc3cc(-c4ccccn4)no3)c(=O)c2=O)CC1] | 1-(2,3-dioxo-4-((3-(pyridin-2- yl)isoxazol-5-yl)methyl)-3,4- dihydropyrazin-1(2H)- yl)cyclopropane-1-carbonitrile | 336 |
| Example 201 | 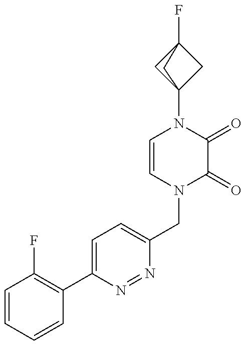 [SMILES: O=c1c(=O)n(C23CC(F)(C2)C3)ccn1Cc1ccc(-c2ccccc2F)nn1] | 1-(3-fluorobicyclo[1.1.1]pentan- 1-yl)-4-((6-(2- fluorophenyl)pyridazin-3- yl)methyl)-1,4-dihydropyrazine- 2,3-dione | 383 |
| Example 202 | 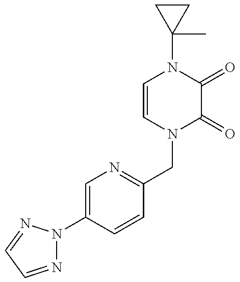 [SMILES: CC1(n2ccn(Cc3ccc(-n4nccn4)cn3)c(=O)c2=O)CC1] | 1-((5-(2H-1,2,3-triazol-2- yl)pyridin-2-yl)methyl)-4-(1- methylcyclopropyl)-1,4- dihydropyrazine-2,3-dione | 325 |
| Example 203 | 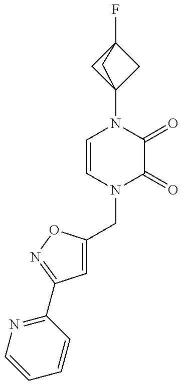 [SMILES: O=c1c(=O)n(C23CC(F)(C2)C3)ccn1Cc1cc(-c2ccccn2)no1] | 1-(3-fluorobicyclo[1.1.1]pentan- 1-yl)-4-((3-(pyridin-2- yl)isoxazol-5-yl)methyl)-1,4- dihydropyrazine-2,3-dione | 355 |
| Example 204 | 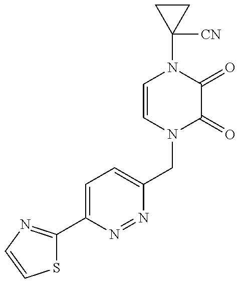 [SMILES: N#CC1(n2ccn(Cc3ccc(-c4nccs4)nn3)c(=O)c2=O)CC1] | 1-(2,3-dioxo-4-((6-(thiazol-2- yl)pyridazin-3-yl)methyl)-3,4- dihydropyrazin-1(2H)- yl)cyclopropane-1-carbonitrile | 353 |
| Example 205 | 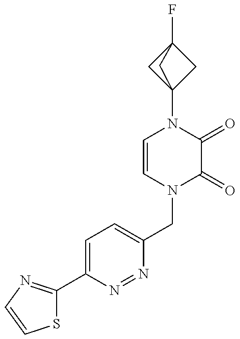 [SMILES: O=c1c(=O)n(C23CC(F)(C2)C3)ccn1Cc1ccc(-c2nccs2)nn1] | 1-(3-fluorobicyclo[1.1.1]pentan- 1-yl)-4-((6-(thiazol-2- yl)pyridazin-3-yl)methyl)-1,4- dihydropyrazine-2,3-dione | 372 |
| Example 206 | 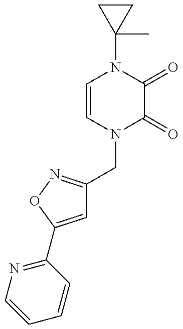 [SMILES: CC1(n2ccn(Cc3cc(-c4ccccn4)on3)c(=O)c2=O)CC1] | 1-(1-methylcyclopropyl)-4-((5- (pyridin-2-yl)isoxazol-3- yl)methyl)-1,4-dihydropyrazine- 2,3-dione | 325 |
| Example 207 | 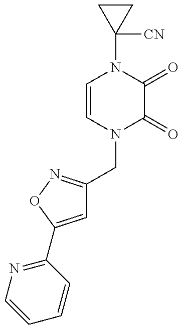 [SMILES: N#CC1(n2ccn(Cc3cc(-c4ccccn4)on3)c(=O)c2=O)CC1] | 1-(2,3-dioxo-4-((5-(pyridin-2- yl)isoxazol-3-yl)methyl)-3,4- dihydropyrazin-1(2H)- yl)cyclopropane-1-carbonitrile | 336 |
| Example 208 | 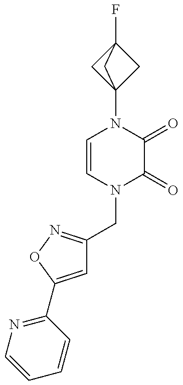 [SMILES: O=c1c(=O)n(C23CC(F)(C2)C3)ccn1Cc1cc(-c2ccccn2)on1] | 1-(3-fluorobicyclo[1.1.1]pentan- 1-yl)-4-((5-(pyridin-2- yl)isoxazol-3-yl)methyl)-1,4- dihydropyrazine-2,3-dione | 355 |
| Example 209 | 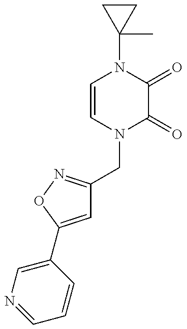 [SMILES: CC1(n2ccn(Cc3cc(-c4cccnc4)on3)c(=O)c2=O)CC1] | 1-(1-methylcyclopropyl)-4-((5- (pyridin-3-yl)isoxazol-3- yl)methyl)-1,4-dihydropyrazine- 2,3-dione | 325 |
| Example 210 | 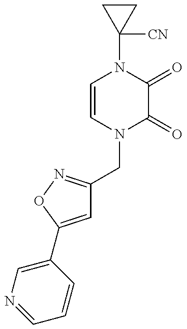 [SMILES: N#CC1(n2ccn(Cc3cc(-c4cccnc4)on3)c(=O)c2=O)CC1] | 1-(2,3-dioxo-4-((5-(pyridin-3- yl)isoxazol-3-yl)methyl)-3,4- dihydropyrazin-1(2H)- yl)cyclopropane-1-carbonitrile | 336 |
| Example 211 | 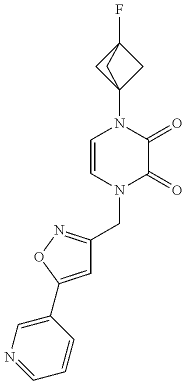 [SMILES: O=c1c(=O)n(C23CC(F)(C2)C3)ccn1Cc1cc(-c2cccnc2)on1] | 1-(3-fluorobicyclo[1.1.1]pentan- 1-yl)-4-((5-(pyridin-3- yl)isoxazol-3-yl)methyl)-1,4- dihydropyrazine-2,3-dione | 355 |
| Example 212 | 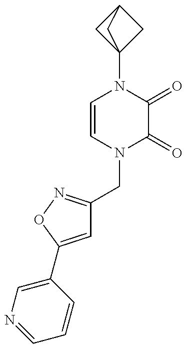 [SMILES: O=c1c(=O)n(C23CC(C2)C3)ccn1Cc1cc(-c2cccnc2)on1] | 1-(bicyclo[1.1.1]pentan-1-yl)-4- ((5-(pyridin-3-yl)isoxazol-3- yl)methyl)-1,4-dihydropyrazine- 2,3-dione | 337 |
| Example 213 | 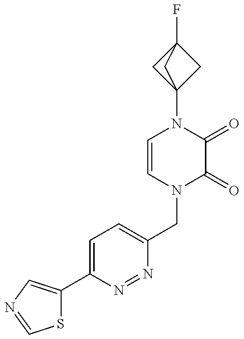 [SMILES: O=c1c(=O)n(C23CC(F)(C2)C3)ccn1Cc1ccc(-c2cncs2)nn1] | 1-(3-fluorobicyclo[1.1.1]pentan- 1-yl)-4-((6-(thiazol-5- yl)pyridazin-3-yl)methyl)-1,4- dihydropyrazine-2,3-dione | 372 |
| Example 214 | 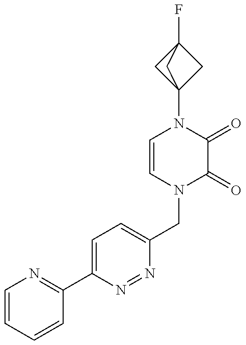 [SMILES: O=c1c(=O)n(C23CC(F)(C2)C3)ccn1Cc1ccc(-c2ccccn2)nn1] | 1-(3-fluorobicyclo[1.1.1]pentan- 1-yl)-4-((6-(pyridin-2- yl)pyridazin-3-yl)methyl)-1,4- dihydropyrazine-2,3-dione | 366 |
| Example 215 | 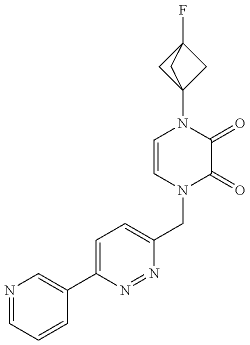 [SMILES: O=c1c(=O)n(C23CC(F)(C2)C3)ccn1Cc1ccc(-c2cccnc2)nn1] | 1-(3-fluorobicyclo[1.1.1]pentan- 1-yl)-4-((6-(pyridin-3- yl)pyridazin-3-yl)methyl)-1,4- dihydropyrazine-2,3-dione | 366 |
| Example 216 | 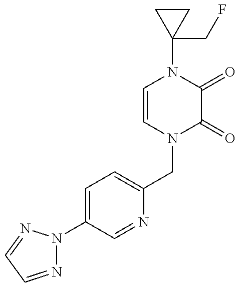 [SMILES: O=c1c(=O)n(C2(CF)CC2)ccn1Cc1ccc(-n2nccn2)cn1] | 1-((5-(2H-1,2,3-triazol-2- yl)pyridin-2-yl)methyl)-4-(1- (fluoromethyl)cyclopropyl)-1,4- dihydropyrazine-2,3-dione | 343 |
| Example 217 | 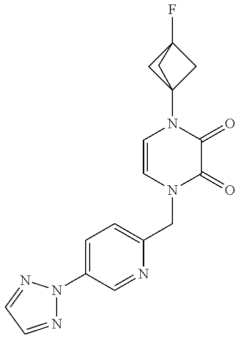 [SMILES: O=c1c(=O)n(C23CC(F)(C2)C3)ccn1Cc1ccc(-n2nccn2)cn1] | 1-((5-(2H-1,2,3-triazol-2- yl)pyridin-2-yl)methyl)-4-(3- fluorobicyclo[1.1.1]pentan-1-yl)- 1,4-dihydropyrazine-2,3-dione | 355 |
| Example 218 | 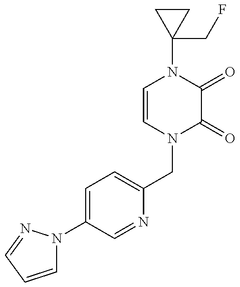 [SMILES: O=c1c(=O)n(C2(CF)CC2)ccn1Cc1ccc(-n2cccn2)cn1] | 1-((5-(1H-pyrazol-1-yl)pyridin- 2-yl)methyl)-4-(1- (fluoromethyl)cyclopropyl)-1,4- dihydropyrazine-2,3-dione | 342 |
| Example 219 | 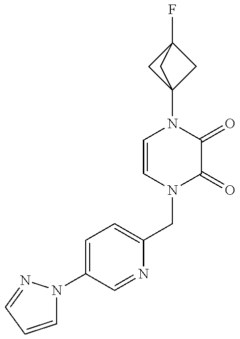 [SMILES: O=c1c(=O)n(C23CC(F)(C2)C3)ccn1Cc1ccc(-n2cccn2)cn1] | 1-((5-(1H-pyrazol-1-yl)pyridin- 2-yl)methyl)-4-(3- fluorobicyclo[1.1.1]pentan-1-yl)- 1,4-dihydropyrazine-2,3-dione | 354 |
| Example 220 | 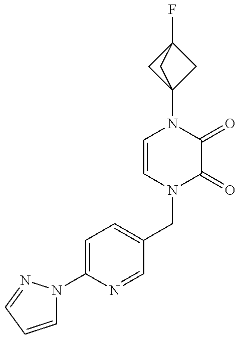 [SMILES: O=c1c(=O)n(C23CC(F)(C2)C3)ccn1Cc1ccc(-n2cccn2)nc1] | 1-((6-(1H-pyrazol-1-yl)pyridin- 3-yl)methyl)-4-(3- fluorobicyclo[1.1.1]pentan-1-yl)- 1,4-dihydropyrazine-2,3-dione | 354 |
| Example 221 | 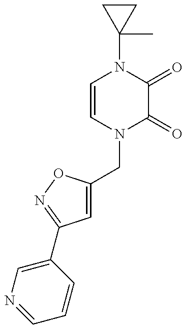 [SMILES: CC1(n2ccn(Cc3cc(-c4cccnc4)no3)c(=O)c2=O)CC1] | 1-(1-methylcyclopropyl)-4-((3- (pyridin-3-yl)isoxazol-5- yl)methyl)-1,4-dihydropyrazine- 2,3-dione | 325 |
| Example 222 | 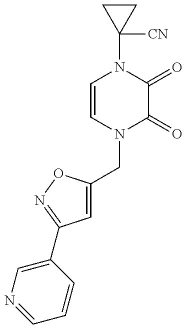 [SMILES: N#CC1(n2ccn(Cc3cc(-c4cccnc4)no3)c(=O)c2=O)CC1] | 1-(2,3-dioxo-4-((3-(pyridin-3- yl)isoxazol-5-yl)methyl)-3,4- dihydropyrazin-1(2H)- yl)cyclopropane-1-carbonitrile | 336 |
| Example 223 | 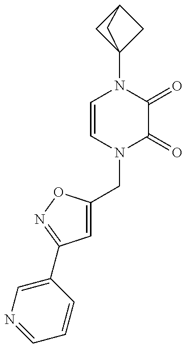 [SMILES: O=c1c(=O)n(C23CC(C2)C3)ccn1Cc1cc(-c2cccnc2)no1] | 1-(bicyclo[1.1.1]pentan-1-yl)-4- ((3-(pyridin-3-yl)isoxazol-5- yl)methyl)-1,4-dihydropyrazine- 2,3-dione | 337 |
| Example 224 | 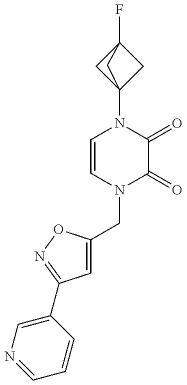 [SMILES: O=c1c(=O)n(C23CC(F)(C2)C3)ccn1Cc1cc(-c2cccnc2)no1] | 1-(3-fluorobicyclo[1.1.1]pentan- 1-yl)-4-((3-(pyridin-3- yl)isoxazol-5-yl)methyl)-1,4- dihydropyrazine-2,3-dione | 355 |
| Example 225 | 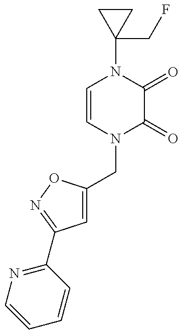 [SMILES: O=c1c(=O)n(C2(CF)CC2)ccn1Cc1cc(-c2ccccn2)no1] | 1-(1-(fluoromethyl)cyclopropyl)- 4-((3-(pyridin-2-yl)isoxazol-5- yl)methyl)-1,4-dihydropyrazine- 2,3-dione | 343 |
| Example 226 | 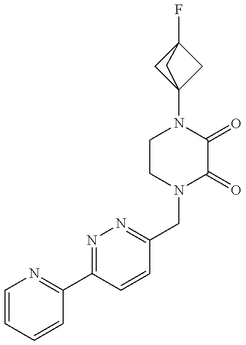 [SMILES: O=C1C(=O)N(C23CC(F)(C2)C3)CCN1Cc1ccc(-c2ccccn2)nn1] | 1-(3-fluorobicyclo[1.1.1]pentan- 1-yl)-4-((6-(pyridin-2- yl)pyridazin-3-yl)methyl)-1,4- dihydropyrazine-2,3-dione | 368 |
| Example 227 | 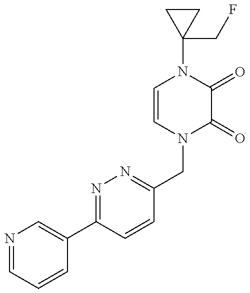 [SMILES: O=c1c(=O)n(C2(CF)CC2)ccn1Cc1ccc(-c2cccnc2)nn1] | 1-(1-(fluoromethyl)cyclopropyl)- 4-((6-(pyridin-3-yl)pyridazin-3- yl)methyl)-1,4-dihydropyrazine- 2,3-dione | 354 |
| Example 228 | 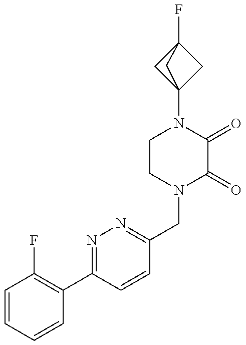 [SMILES: O=C1C(=O)N(C23CC(F)(C2)C3)CCN1Cc1ccc(-c2ccccc2F)nn1] | 1-(3-fluorobicyclo[1.1.1]pentan- 1-yl)-4-((6-(2- fluorophenyl)pyridazin-3- yl)methyl)piperazine-2,3-dione | 385 |
| Example 229 | 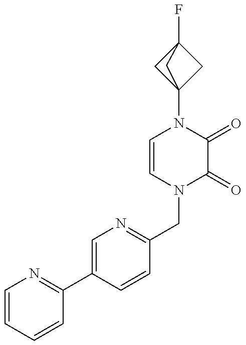 [SMILES: O=c1c(=O)n(C23CC(F)(C2)C3)ccn1Cc1ccc(-c2ccccn2)cn1] | 1-([2,3′-bipyridin]-6′-ylmethyl)- 4-(3-fluorobicyclo[1.1.1]pentan- 1-yl)-1,4-dihydropyrazine-2,3- dione | 365 |
| Example 230 | 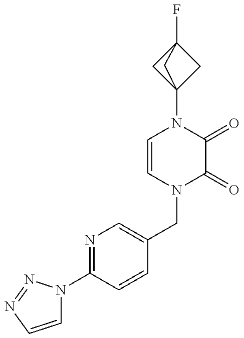 [SMILES: O=c1c(=O)n(C23CC(F)(C2)C3)ccn1Cc1ccc(-n2ccnn2)nc1] | 1-((6-(1H-1,2,3-triazol-1- yl)pyridin-3-yl)methyl)-4-(3- fluorobicyclo[1.1.1]pentan-1-yl)- 1,4-dihydropyrazine-2,3-dione | 355 |
| Example 231 | 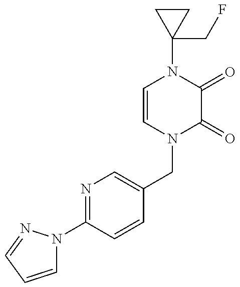 [SMILES: O=c1c(=O)n(C2(CF)CC2)ccn1Cc1ccc(-n2cccn2)nc1] | 1-((6-(1H-pyrazol-1-yl)pyridin- 3-yl)methyl)-4-(1- (fluoromethyl)cyclopropyl)-1,4- dihydropyrazine-2,3-dione | 342 |
| Example 232 | 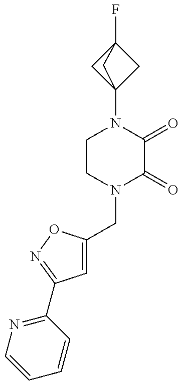 [SMILES: O=C1C(=O)N(C23CC(F)(C2)C3)CCN1Cc1cc(-c2ccccn2)no1] | 1-(3-fluorobicyclo[1.1.1]pentan- 1-yl)-4-((3-(pyridin-2- yl)isoxazol-5- yl)methyl)piperazine-2,3-dione | 357 |
| Example 233 | 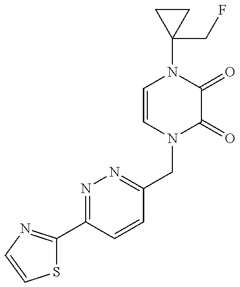 [SMILES: O=c1c(=O)n(C2(CF)CC2)ccn1Cc1ccc(-c2nccs2)nn1] | 1-(1-(fluoromethyl)cyclopropyl)- 4-((6-(thiazol-2-yl)pyridazin-3- yl)methyl)-1,4-dihydropyrazine- 2,3-dione | 360 |
| Example 234 | 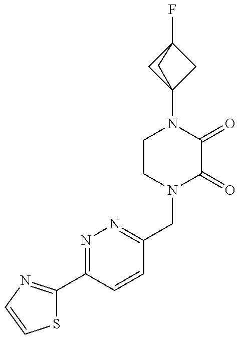 [SMILES: O=C1C(=O)N(C23CC(F)(C2)C3)CCN1Cc1ccc(-c2nccs2)nn1] | 1-(3-fluorobicyclo[1.1.1]pentan- 1-yl)-4-((6-(thiazol-2- yl)pyridazin-3- yl)methyl)piperazine-2,3-dione | 374 |
| Example 235 | 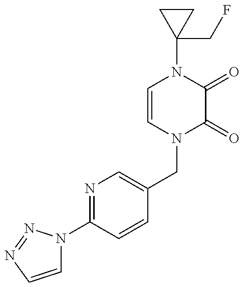 [SMILES: O=c1c(=O)n(C2(CF)CC2)ccn1Cc1ccc(-n2ccnn2)nc1] | 1-((6-(1H-1,2,3-triazol-1- yl)pyridin-3-yl)methyl)-4-(1- (fluoromethyl)cyclopropyl)-1,4- dihydropyrazine-2,3-dione | 343 |
| Example 236 | 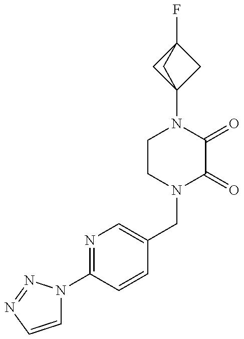 [SMILES: O=C1C(=O)N(C23CC(F)(C2)C3)CCN1Cc1ccc(-n2ccnn2)nc1] | 1-((6-(1H-1,2,3-triazol-1- yl)pyridin-3-yl)methyl)-4-(3- fluorobicyclo[1.1.1]pentan-1- yl)piperazine-2,3-dione | 357 |
| Example 237 | 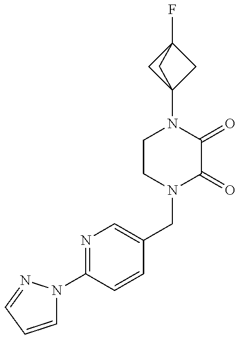 [SMILES: O=C1C(=O)N(C23CC(F)(C2)C3)CCN1Cc1ccc(-n2cccn2)nc1] | 1-((6-(1H-pyrazol-1-yl)pyridin- 3-yl)methyl)-4-(3- fluorobicyclo[1.1.1]pentan-1- yl)piperazine-2,3-dione | 356 |
| Example 238 | 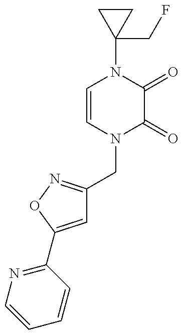 [SMILES: O=c1c(=O)n(C2(CF)CC2)ccn1Cc1cc(-c2ccccn2)on1] | 1-(1-(fluoromethyl)cyclopropyl)- 4-((5-(pyridin-2-yl)isoxazol-3- yl)methyl)-1,4-dihydropyrazine- 2,3-dione | 343 |
| Example 239 | 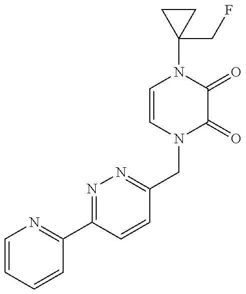 [SMILES: O=c1c(=O)n(C2(CF)CC2)ccn1Cc1ccc(-c2ccccn2)nn1] | 1-(1-(fluoromethyl)cyclopropyl)- 4-((6-(pyridin-2-yl)pyridazin-3- yl)methyl)-1,4-dihydropyrazine- 2,3-dione | 354 |
| Example 240 | 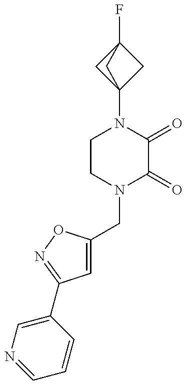 [SMILES: O=C1C(=O)N(C23CC(F)(C2)C3)CCN1Cc1cc(-c2cccnc2)no1] | 1-(3-fluorobicyclo[1.1.1]pentan- 1-yl)-4-((3-(pyridin-3- yl)isoxazol-5- yl)methyl)piperazine-2,3-dione | 357 |
| Example 241 | 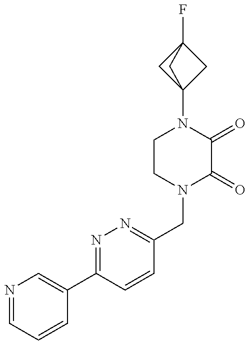 [SMILES: O=C1C(=O)N(C23CC(F)(C2)C3)CCN1Cc1ccc(-c2cccnc2)nn1] | 1-(3-fluorobicyclo[1.1.1]pentan- 1-yl)-4-((6-(pyridin-3- yl)pyridazin-3- yl)methyl)piperazine-2,3-dione | 368 |
| Example 242 | 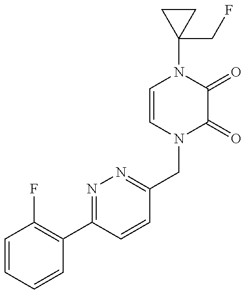 [SMILES: O=c1c(=O)n(C2(CF)CC2)ccn1Cc1ccc(-c2ccccc2F)nn1] | 1-(1-(fluoromethyl)cyclopropyl)- 4-((6-(2-fluorophenyl)pyridazin- 3-yl)methyl)-1,4- dihydropyrazine-2,3-dione | 371 |
| Example 243 | 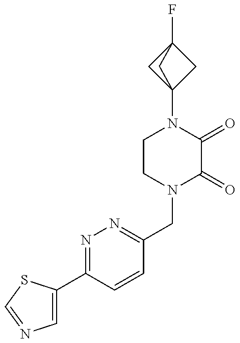 [SMILES: O=C1C(=O)N(C23CC(F)(C2)C3)CCN1Cc1ccc(-c2cncs2)nn1] | 1-(3-fluorobicyclo[1.1.1]pentan- 1-yl)-4-((6-(thiazol-5- yl)pyridazin-3- yl)methyl)piperazine-2,3-dione | 374 |
| Example 244 | 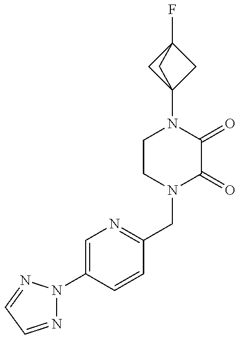 [SMILES: O=C1C(=O)N(C23CC(F)(C2)C3)CCN1Cc1ccc(-n2nccn2)cn1] | 1-((5-(2H-1,2,3-triazol-2- yl)pyridin-2-yl)methyl)-4-(3- fluorobicyclo[1.1.1]pentan-1- yl)piperazine-2,3-dione | 357 |
| Example 245 | 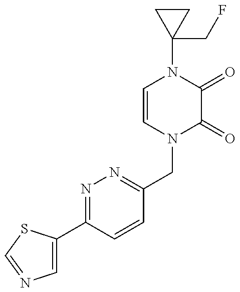 [SMILES: O=c1c(=O)n(C2(CF)CC2)ccn1Cc1ccc(-c2cncs2)nn1] | 1-(1-(fluoromethyl)cyclopropyl)- 4-((6-(thiazol-5-yl)pyridazin-3- yl)methyl)-1,4-dihydropyrazine- 2,3-dione | 360 |
| Example 246 | 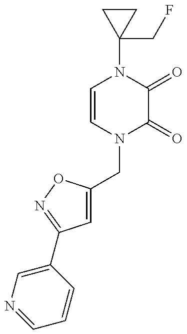 [SMILES: O=c1c(=O)n(C2(CF)CC2)ccn1Cc1cc(-c2cccnc2)no1] | 1-(1-(fluoromethyl)cyclopropyl)- 4-((3-(pyridin-3-yl)isoxazol-5- yl)methyl)-1,4-dihydropyrazine- 2,3-dione | 343 |
| Example 247 | 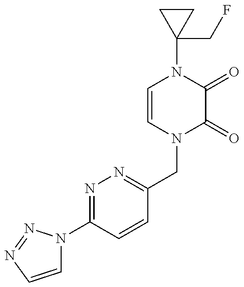 [SMILES: O=c1c(=O)n(C2(CF)CC2)ccn1Cc1ccc(-n2ccnn2)nn1] | 1-((6-(1H-1,2,3-triazol-1- yl)pyridazin-3-yl)methyl)-4-(1- (fluoromethyl)cyclopropyl)-1,4- dihydropyrazine-2,3-dione | 344 |
| Example 248 | 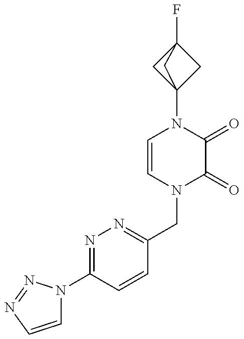 [SMILES: O=c1c(=O)n(C23CC(F)(C2)C3)ccn1Cc1ccc(-n2ccnn2)nn1] | 1-((6-(1H-1,2,3-triazol-1- yl)pyridazin-3-yl)methyl)-4-(3- fluorobicyclo[1.1.1]pentan-1-yl)- 1,4-dihydropyrazine-2,3-dione | 356 |
| Example 249 | 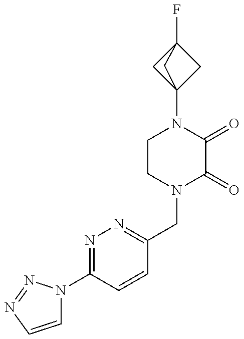 [SMILES: O=C1C(=O)N(C23CC(F)(C2)C3)CCN1Cc1ccc(-n2ccnn2)nn1] | 1-((6-(1H-1,2,3-triazol-1- yl)pyridazin-3-yl)methyl)-4-(3- fluorobicyclo[1.1.1]pentan-1- yl)piperazine-2,3-dione | 358 |
| Example 250 | 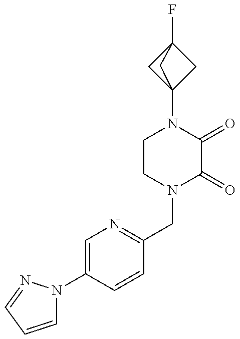 [SMILES: O=C1C(=O)N(C23CC(F)(C2)C3)CCN1Cc1ccc(-n2cccn2)cn1] | 1-((5-(1H-pyrazol-1-yl)pyridin- 2-yl)methyl)-4-(3- fluorobicyclo[1.1.1]pentan-1- yl)piperazine-2,3-dione | 356 |
| Example 251 | 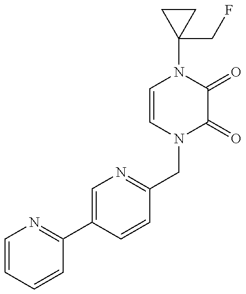 [SMILES: O=c1c(=O)n(C2(CF)CC2)ccn1Cc1ccc(-c2ccccn2)cn1] | 1-([2,3′-bipyridin]-6′-ylmethyl)- 4-(1-(fluoromethyl)cyclopropyl)- 1,4-dihydropyrazine-2,3-dione | 353 |
| Example 252 | 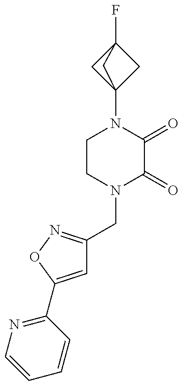 [SMILES: O=C1C(=O)N(C23CC(F)(C2)C3)CCN1Cc1cc(-c2ccccn2)on1] | 1-(3-fluorobicyclo[1.1.1]pentan- 1-yl)-4-((5-(pyridin-2- yl)isoxazol-3- yl)methyl)piperazine-2,3-dione | 357 |
| Example 253 | 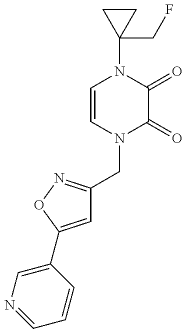 [SMILES: O=c1c(=O)n(C2(CF)CC2)ccn1Cc1cc(-c2cccnc2)on1] | 1-(1-(fluoromethyl)cyclopropyl)- 4-((5-(pyridin-3-yl)isoxazol-3- yl)methyl)-1,4-dihydropyrazine- 2,3-dione | 343 |
| Example 254 | 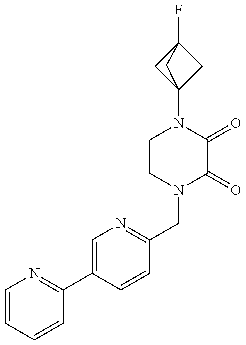 [SMILES: O=C1C(=O)N(C23CC(F)(C2)C3)CCN1Cc1ccc(-c2ccccn2)cn1] | 1-([2,3′-bipyridin]-6′-ylmethyl)- 4-(3-fluorobicyclo[1.1.1]pentan- 1-yl)piperazine-2,3-dione | 367 |
| Example 255 | 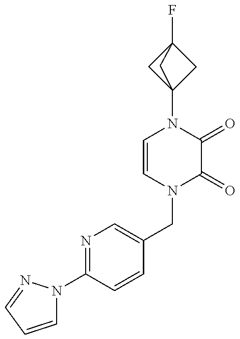 [SMILES: O=c1c(=O)n(C23CC(F)(C2)C3)ccn1Cc1ccc(-n2cccn2)nc1] | 1-((6-(2H-1,2,3-triazol-2- yl)pyridin-3-yl)methyl)-4-(3- fluorobicyclo[1.1.1]pentan-1-yl)- 1,4-dihydropyrazine-2,3-dione | 355 |
| Example 256 | 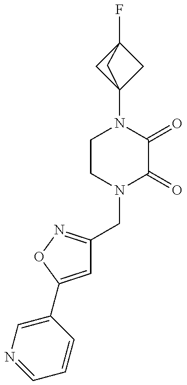 [SMILES: O=C1C(=O)N(C23CC(F)(C2)C3)CCN1Cc1cc(-c2cccnc2)on1] | 1-(3-fluorobicyclo[1.1.1]pentan- 1-yl)-4-((5-(pyridin-3- yl)isoxazol-3- yl)methyl)piperazine-2,3-dione | 357 |
| Example 257 | 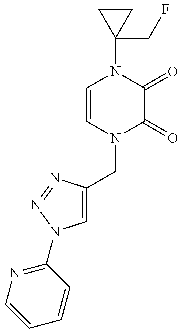 [SMILES: O=c1c(=O)n(C2(CF)CC2)ccn1Cc1cn(-c2ccccn2)nn1] | 1-(1-(fluoromethyl)cyclopropyl)- 4-((1-(pyridin-2-yl)-1H-1,2,3- triazol-4-yl)methyl)-1,4- dihydropyrazine-2,3-dione | 343 |
| Example 258 | 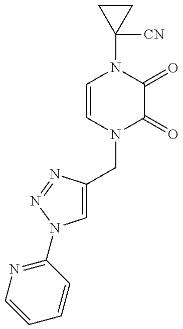 [SMILES: N#CC1(n2ccn(Cc3cn(-c4ccccn4)nn3)c(=O)c2=O)CC1] | 1-(2,3-dioxo-4-((1-(pyridin-2- yl)-1H-1,2,3-triazol-4- yl)methyl)-3,4-dihydropyrazin- 1(2H)-yl)cyclopropane-1- carbonitrile | 336 |
| Example 259 | 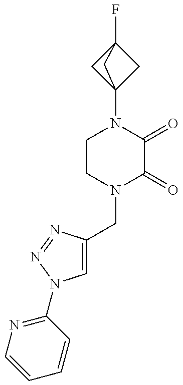 [SMILES: O=C1C(=O)N(C23CC(F)(C2)C3)CCN1Cc1cn(-c2ccccn2)nn1] | 1-(3-fluorobicyclo[1.1.1]pentan- 1-yl)-4-((1-(pyridin-2-yl)-1H- 1,2,3-triazol-4- yl)methyl)piperazine-2,3-dione | 357 |
| Example 260 | 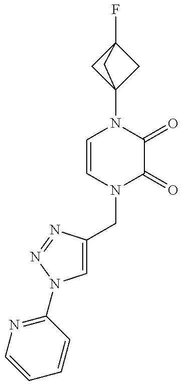 [SMILES: O=c1c(=O)n(C23CC(F)(C2)C3)ccn1Cc1cn(-c2ccccn2)nn1] | 1-(3-fluorobicyclo[1.1.1]pentan- 1-yl)-4-((1-(pyridin-2-yl)-1H- 1,2,3-triazol-4-yl)methyl)-1,4- dihydropyrazine-2,3-dione | 355 |
| Example 261 | 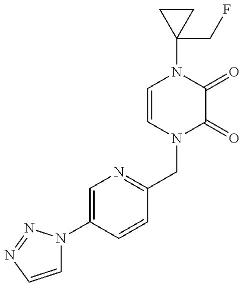 [SMILES: O=c1c(=O)n(C2(CF)CC2)ccn1Cc1ccc(-n2ccnn2)cn1] | 1-((5-(1H-1,2,3-triazol-1- yl)pyridin-2-yl)methyl)-4-(1- (fluoromethyl)cyclopropyl)-1,4- dihydropyrazine-2,3-dione | 343 |
| Example 262 | 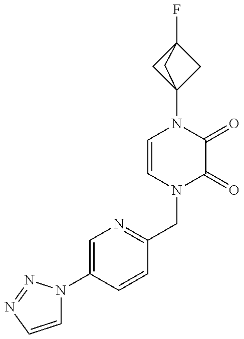 [SMILES: O=c1c(=O)n(C23CC(F)(C2)C3)ccn1Cc1ccc(-n2ccnn2)cn1] | 1-((5-(1H-1,2,3-triazol-1- yl)pyridin-2-yl)methyl)-4-(3- fluorobicyclo[1.1.1]pentan-1-yl)- 1,4-dihydropyrazine-2,3-dione | 355 |
| Example 263 | 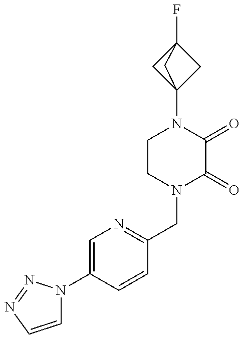 [SMILES: O=C1C(=O)N(C23CC(F)(C2)C3)CCN1Cc1ccc(-n2ccnn2)cn1] | 1-((5-(1H-1,2,3-triazol-1- yl)pyridin-2-yl)methyl)-4-(3- fluorobicyclo[1.1.1]pentan-1- yl)piperazine-2,3-dione | 357 |
| Example 264 | 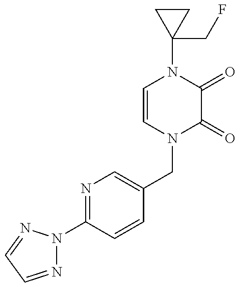 [SMILES: O=c1c(=O)n(C2(CF)CC2)ccn1Cc1ccc(-n2nccn2)nc1] | 1-((6-(2H-1,2,3-triazol-2- yl)pyridin-3-yl)methyl)-4-(1- (fluoromethyl)cyclopropyl)-1,4- dihydropyrazine-2,3-dione | 343 |
| Example 265 | 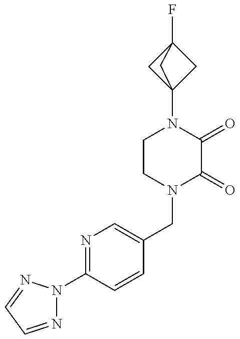 [SMILES: O=C1C(=O)N(C23CC(F)(C2)C3)CCN1Cc1ccc(-n2nccn2)nc1] | 1-((6-(2H-1,2,3-triazol-2- yl)pyridin-3-yl)methyl)-4-(3- fluorobicyclo[1.1.1]pentan-1- yl)piperazine-2,3-dione | 357 |

### Examples 266A and 266B

### Preparation of cis-1-(3-phenylcyclobutyl)-4-((6-phenylpyridin-3-yl)methyl)piperazine-2,3-dione and trans-1-(3-phenylcyclobutyl)-4-((6-phenylpyridin-3-yl)methyl)piperazine-2,3-dione

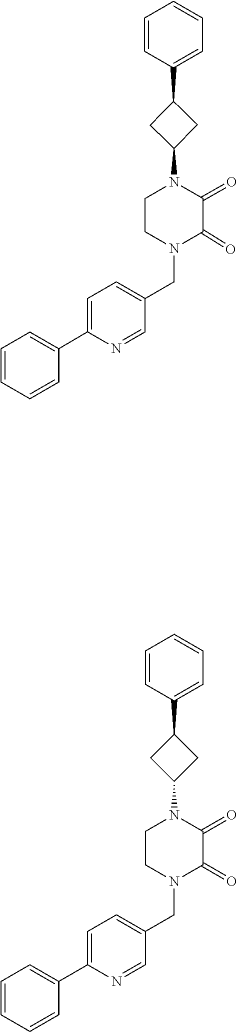 [SMILES: O=C1C(=O)N([C@H]2C[C@@H](c3ccccc3)C2)CCN1Cc1ccc(-c2ccccc2)nc1 ; O=C1C(=O)N([C@H]2C[C@H](c3ccccc3)C2)CCN1Cc1ccc(-c2ccccc2)nc1]

The isomeric mixture of 1-(3-phenylcyclobutyl)-4-((6-phenylpyridin-3-yl)methyl)piperazine-2,3-dione was resolved by chiral SFC purification (CCA 21×250 mm column, 45% MeOH w/ 0.1% NH₄OH as cosolvent) to afford cis-1-(3-phenylcyclobutyl)-4-((6-phenylpyridin-3-yl)methyl)piperazine-2,3-dione as the first eluting peak and trans-1-(3-phenylcyclobutyl)-4-((6-phenylpyridin-3-yl)methyl)piperazine-2,3-dione as the second eluting peak.

Peak 1: ¹H NMR (500 MHz, DMSO-d₆) δ 8.63 (d, J=1.7 Hz, 1H), 8.08 (d, J=7.3 Hz, 2H), 7.96 (d, J=8.2 Hz, 1H), 7.82 (dd, J=8.2, 2.1 Hz, 1H), 7.50 (t, J=7.4 Hz, 2H), 7.44 (t, J=7.2 Hz, 1H), 7.34-7.28 (m, 4H), 7.22-7.17 (m, 1H), 4.78-4.66 (m, 1H), 4.66 (s, 2H), 3.62 (dd, J=7.2, 4.0 Hz, 2H), 3.57 (dd, J=7.0, 3.8 Hz, 2H), 3.23-3.16 (m, 1H), 2.56-2.48 (m, 2H), 2.30-2.20 (m, 2H). LC/MS (m/z): 412 (M+H)⁺

Peak 2: ¹H NMR (500 MHz, DMSO-d₆) δ 8.64 (d, J=1.8 Hz, 1H), 8.11-8.06 (m, 2H), 7.97 (d, J=8.2 Hz, 1H), 7.83 (dd, J=8.2, 2.2 Hz, 1H), 7.50 (t, J=7.4 Hz, 2H), 7.44 (t, J=7.3 Hz, 1H), 7.38-7.32 (m, 4H), 7.24-7.20 (m, 1H), 4.98 (p, J=8.6 Hz, 1H), 4.67 (s, 2H), 3.72 (dd, J=7.1, 4.3 Hz, 2H), 3.61 (dd, J=7.0, 4.3 Hz, 2H), 3.51-3.46 (m, 1H), 2.76-2.66 (m, 2H), 2.37-2.30 (m, 2H). LC/MS (m/z): 412 (M+H)⁺

### Example 267

### Preparation of 5-((4-cyclopentyl-2,3-dioxopiperazin-1-yl)methyl)-2-phenylpyridine 1-oxide

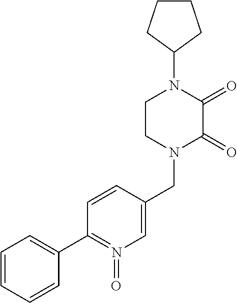 [SMILES: O=C1C(=O)N(C2CCCC2)CCN1Cc1ccc(-c2ccccc2)[n+]([O-])c1]

meta-Chloroperoxybenzoic acid (m-CPBA) (50 mg, 0.3 mmol, 85% wt) was added to a mixture of 1-cyclopentyl-4-((6-phenylpyridin-3-yl)methyl)piperazine-2,3-dione (32 mg, 0.091 mmol) in DCM (3 mL) at room temperature. The reaction mixture was stirred at room temperature for 12 hours. The mixture was concentrated under reduced pressure, and the residue was purified by silica gel chromatography (eluting methanol in dichloromethane) to afford 5-((4-cyclopentyl-2,3-dioxopiperazin-1-yl)methyl)-2-phenylpyridine 1-oxide. ¹H NMR (400 MHz, methanol-d₄) δ 8.44 (s, 1H), 7.96 (s, 1H), 7.91 (d, J=7.8 Hz, 1H), 7.81-7.76 (m, 2H), 7.67-7.61 (m, 2H), 7.47-7.38 (m, 1H), 4.83-4.77 (m, 1H), 4.72 (s, 2H), 3.69-3.62 (m, 2H), 3.61-3.54 (m, 2H), 1.97-1.85 (m, 2H), 1.81-1.71 (m, 2H), 1.70-1.54 (m, 4H). LC/MS (m/z): 366 (M+H)⁺

### Example 268

### Preparation of 1-(2-(7-chlorocinnolin-3-yl)ethyl)-4-cyclopentylpiperazine-2,3-dione

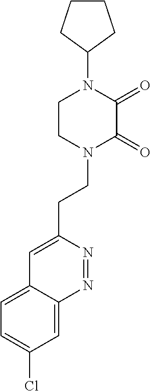 [SMILES: O=C1C(=O)N(C2CCCC2)CCN1CCc1cc2ccc(Cl)cc2nn1]

### Step A: 7-chlorocinnolin-3-yl trifluoromethanesulfonate

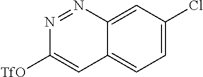 [SMILES: O=S(=O)(Oc1cc2ccc(Cl)cc2nn1)C(F)(F)F]

Triflic anhydride (0.42 mL, 2.49 mmol) was added to a mixture of TEA (0.93 mL, 6.6 mmol) and 7-chlorocinnolin-3-ol (0.300 g, 1.66 mmol) in DCM (10 mL) at 0° C. The reaction mixture was warmed to room temperature and stirred for 2 hours. The mixture was concentrated under reduced pressure. The residue was purified by silica gel chromatography (eluting ethyl acetate in petroleum ether) to afford 7-chlorocinnolin-3-yl trifluoromethanesulfonate. LC/MS (m/z): 313 (M+H)⁺

### Step B: 7-chloro-3-vinylcinnoline

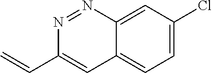

A mixture of 2,4,6-trivinylcyclotriboroxane pyridine complex (51 mg, 0.21 mmol), 7-chlorocinnolin-3-yl trifluoromethanesulfonate (220 mg, 0.71 mmol) and potassium carbonate (243 mg, 1.76 mmol) was flushed with nitrogen. 1,4-dioxane (10 mL) and water (1 mL) were added, and the mixture was sparged with nitrogen. Palladium-tetrakis(triphenylphosphine) (65 mg, 0.056 mmol) was added, and the reaction mixture was stirred and heated at 80° C. for 12 hours under a nitrogen atmosphere. The mixture was cooled to room temperature, diluted with ethyl acetate (20 mL), and washed with saturated aqueous sodium carbonate (20 mL). The organic layer was separated, dried over anhydrous sodium sulfate, filtered, and concentrated under reduced pressure. The residue was purified by silica gel chromatography (eluting ethyl acetate in petroleum ether) to afford 7-chloro-3-vinylcinnoline. LC/MS (m/z): 191 (M+H)⁺

### Step C: 1-(2-(7-chlorocinnolin-3-yl)ethyl)-4-cyclopentylpiperazine-2,3-dione

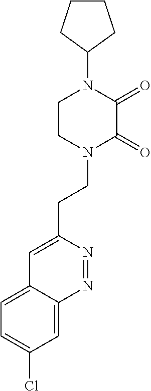 [SMILES: O=C1C(=O)N(C2CCCC2)CCN1CCc1cc2ccc(Cl)cc2nn1]

Potassium carbonate (174 mg, 1.26 mmol) was added to a mixture of 1-cyclopentylpiperazine-2,3-dione (92 mg, 0.50 mmol) and 7-chloro-3-vinylcinnoline (80 mg, 0.42 mmol) in N-methyl-2-pyrrolidone (3 mL) at room temperature. The reaction mixture was stirred and heated at 80° C. for 12 hours under a nitrogen atmosphere. The mixture was cooled to room temperature, diluted with ethyl acetate (20 mL), and washed with water (50 mL). The organic layer was separated, dried over anhydrous sodium sulfate, filtered, and concentrated under reduced pressure. The residue was purified by reverse phase HPLC (eluting acetonitrile in water, with TFA modifier) to afford 1-(2-(7-chlorocinnolin-3-yl)ethyl)-4-cyclopentylpiperazine-2,3-dione. ¹H NMR (500 MHz, methanol-d₄) δ 8.46 (s, 1H), 8.23 (s, 1H), 8.04 (d, J=8.8 Hz, 1H), 7.86 (dd, J=8.9, 2.0 Hz 1H), 4.77-4.74 (m, 1H), 4.02 (t, J=6.8 Hz, 2H), 3.65 (s, 2H), 3.58-3.51 (m, 4H), 1.91-1.84 (m, 2H), 1.77-1.76 (m, 2H), 1.68-1.58 (m, 4H). LC/MS (m/z): 373 (M+H)⁺

### Example 269

### Preparation of 1-(2-(cinnolin-3-yl)ethyl)-4-cyclopentylpiperazine-2,3-dione

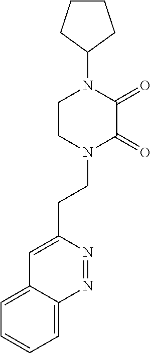 [SMILES: O=C1C(=O)N(C2CCCC2)CCN1CCc1cc2ccccc2nn1]

### Step A: 1-cyclopentyl-4-(2-(1,2-dihydrocinnolin-3-yl)ethyl)piperazine-2,3-dione

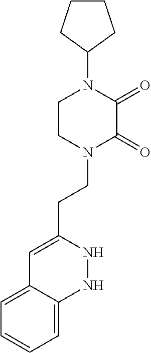 [SMILES: O=C1C(=O)N(C2CCCC2)CCN1CCC1=Cc2ccccc2NN1]

Palladium on carbon (10% w/w, 4 mg, 4 μmol) was added to a mixture of 1-(2-(7-chlorocinnolin-3-yl)ethyl)-4-cyclopentylpiperazine-2,3-dione (15 mg, 0.040 mmol) in ammonia (30% aqueous, 0.05 mL) and MeOH (2.5 mL) under a nitrogen atmosphere at room temperature. The reaction mixture was degassed and backfilled with H₂ (three times). The reaction mixture was stirred under a hydrogen atmosphere at 30° C. for 3 hours. The mixture was filtered, and the filtrate was concentrated under reduced pressure to afford 1-cyclopentyl-4-(2-(1,2-dihydrocinnolin-3-yl)ethyl)piperazine-2,3-dione which was used in the next step without purification. LC/MS (m/z): 341 (M+H)⁺

### Step B: 1-(2-(cinnolin-3-yl)ethyl)-4-cyclopentylpiperazine-2,3-dione

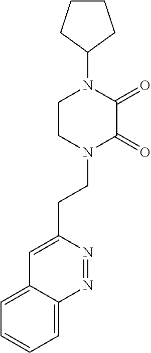 [SMILES: O=C1C(=O)N(C2CCCC2)CCN1CCc1cc2ccccc2nn1]

Manganese dioxide (5 mg, 0.06 mmol) was added to a mixture of 1-cyclopentyl-4-(2-(1,2-dihydrocinnolin-3-yl)ethyl)piperazine-2,3-dione (13 mg, 0.038 mmol) in DCM (2 mL). The reaction mixture was stirred at room temperature for 4 hours under a nitrogen atmosphere. The mixture was filtered, and the filtrate was concentrated under reduced pressure. The residue was purified by reverse phase HPLC (eluting acetonitrile in water, with TFA modifier) to afford 1-(2-(cinnolin-3-yl)ethyl)-4-cyclopentylpiperazine-2,3-dione. ¹H NMR (500 MHz, methanol-d₄) δ 8.45 (d, J=8.5 Hz, 1H), 8.36 (s, 1H), 8.07 (d, J=8.2 Hz, 1H), 8.03-7.98 (m, 1H), 7.96-7.92 (m, 1H), 4.78-4.72 (m, 1H), 4.03 (t, J=6.9 Hz, 2H), 3.70-3.62 (m, 2H), 3.59-3.51 (m, 4H), 1.92-1.84 (m, 2H), 1.81-1.73 (m, 2H), 1.68-1.56 (m, 4H). LC/MS (m/z): 339 (M+H)⁺

### Example 270

### Preparation of 1-cyclopentyl-4-((2-phenylpyrimidin-5-yl)methyl)piperazine-2,3-dione

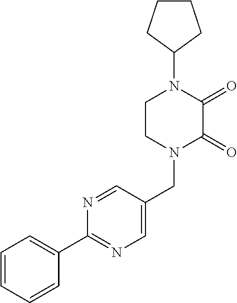 [SMILES: O=C1C(=O)N(C2CCCC2)CCN1Cc1cnc(-c2ccccc2)nc1]

A mixture of 5-bromo-2-phenylpyrimidine (117 mg, 0.499 mmol), 2-(4-cyclopentyl-2,3-dioxopiperazin-1-yl)acetic acid (100 mg, 0.4 mmol), Iridium(III) bis[2-(2,4-difluorophenyl)-5-methylpyridine-N,C20]-4,40-di-tert-butyl-2,20-bipyridine hexafluorophosphate (Ir[dF(Me)ppy]₂(dtbpy)PF₆) (4 mg, 4 μmol), picolinimidamide hydrochloride (1 mg, 8 μmol), nickel(II) bromide ethylene glycol dimethyl ether (3 mg, 8 μmol) and K₃PO₄ (265 mg, 1.25 mmol) in DMF (3 mL) was degassed and backfilled with nitrogen (three times). The reaction mixture was irradiated by 34 W blue LEDs (Kessil lamps) at room temperature for 18 hours. The reaction mixture was filtered, and the filtrate was concentrated under reduced pressure. The residue was purified by reverse phase HPLC (eluting acetonitrile in water, with ammonium bicarbonate modifier) to afford 1-cyclopentyl-4-((2-phenylpyrimidin-5-yl)methyl)piperazine-2,3-dione. ¹H NMR (400 MHz, methanol-d₄) δ 8.86 (s, 2H), 8.43-8.38 (m, 2H), 7.50-7.49 (m, 3H), 4.84-4.77 (m, 1H), 4.72 (s, 2H), 3.69-3.63 (m, 2H), 3.58-3.53 (m, 2H), 1.95-1.84 (m, 2H), 1.77-1.74 (m, 2H), 1.69-1.56 (m, 4H). LC/MS (m/z): 351 (M+H)⁺

Examples shown in Example Table 4 below, were or may be prepared according to procedures analogous to those outlined in Example 270 above using the appropriate starting materials, described in the Preparations or Intermediates above, or as obtained from commercial

Example Table 4

| | | | Mass |
|---|---|---|---|
| Example | Structure | Name | [M + H]⁺ |
| Example 271 | 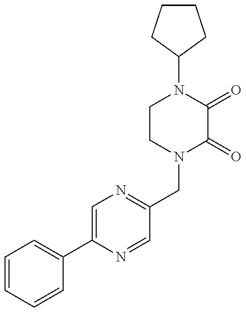 [SMILES: O=C1C(=O)N(C2CCCC2)CCN1Cc1cnc(-c2ccccc2)cn1] | 1-cyclopentyl-4-((5- phenylpyrazin-2- yl)methyl)piperazine-2,3-dione | 351 |
| Example 272 | 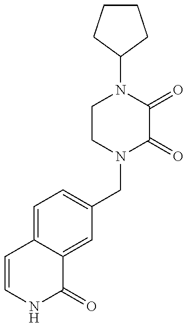 [SMILES: O=C1C(=O)N(C2CCCC2)CCN1Cc1ccc2cc[nH]c(=O)c2c1] | 1-cyclopentyl-4-((1-oxo-1,2- dihydroisoquinolin-7- yl)methyl)piperazine-2,3-dione | 340 |
| Example 273 | 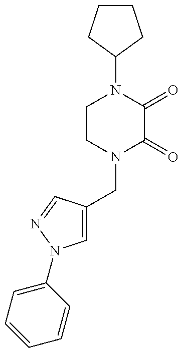 [SMILES: O=C1C(=O)N(C2CCCC2)CCN1Cc1cnn(-c2ccccc2)c1] | 1-cyclopentyl-4-((1-phenyl-1H- pyrazol-4-yl)methyl)piperazine- 2,3-dione | 339 |
| Example 274 | 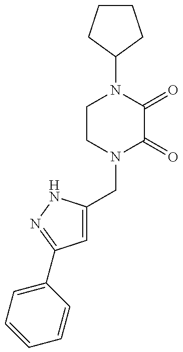 [SMILES: O=C1C(=O)N(C2CCCC2)CCN1Cc1cc(-c2ccccc2)n[nH]1] | 1-cyclopentyl-4-((3-phenyl-1H- pyrazol-5-yl)methyl)piperazine- 2,3-dione | 339 |
| Example 275 | 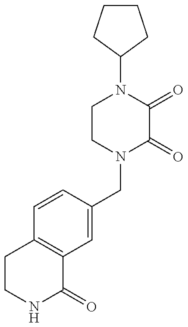 [SMILES: O=C1NCCc2ccc(CN3CCN(C4CCCC4)C(=O)C3=O)cc21] | 1-cyclopentyl-4-((1-oxo-1,2,3,4- tetrahydroisoquinolin-7- yl)methyl)piperazine-2,3-dione | 342 |

### Example 276

### Preparation of 1-(bicyclo[1.1.1]pentan-1-yl)-4-((5-phenylpyrimidin-2-yl)methyl)piperazine-2,3-dione

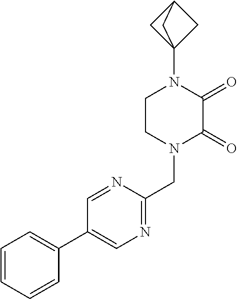 [SMILES: O=C1C(=O)N(C23CC(C2)C3)CCN1Cc1ncc(-c2ccccc2)cn1]

Bicyclo[1.1.1]pentan-1-amine (14 mg, 0.17 mmol) and sodium triacetoxyborohydride (97 mg, 0.46 mmol) were added to a mixture of ethyl 2-oxo-2-((2-oxoethyl)((5-phenylpyrimidin-2-yl)methyl)amino)acetate (0.050 g, 0.15 mmol) in 1,2-dichloroethane (2 mL) at room temperature. The reaction mixture was stirred at room temperature for 16 hours. The reaction mixture was poured into water (10 mL) and then extracted with DCM (3×10 mL). The organic layers were combined, dried over anhydrous sodium sulfate, filtered, and concentrated under reduced pressure. The residue was purified by reverse phase HPLC (eluting acetonitrile in water, with ammonium bicarbonate modifier) to afford 1-(bicyclo[1.1.1]pentan-1-yl)-4-((5-phenylpyrimidin-2-yl)methyl)piperazine-2,3-dione. ¹H NMR (400 MHz, methanol-d₄) δ 9.02 (s, 2H), 7.71 (d, J=7.1 Hz, 2H), 7.56-7.51 (m, 2H), 7.50-7.44 (m, 1H), 4.94 (s, 2H), 3.85-3.76 (m, 2H), 3.75-3.67 (m, 2H), 2.54 (s, 1H), 2.25 (s, 6H). LC/MS (m/z): 349 (M+H)⁺

Examples shown in Example Table 5 below, were or may be prepared according to procedures analogous to those outlined in Example 276 above using the appropriate starting materials, described in the Preparations or Intermediates above, or as obtained from commercial

Example Table 5

| | | | Mass |
|---|---|---|---|
| Example | Structure | Name | [M + H]⁺ |
| Example 277 | 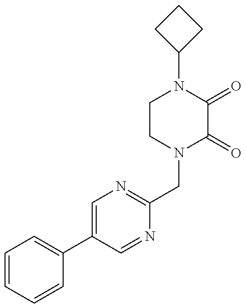 [SMILES: O=C1C(=O)N(C2CCC2)CCN1Cc1ncc(-c2ccccc2)cn1] | 1-cyclobutyl-4-((5- phenylpyrimidin-2- yl)methyl)piperazine-2,3-dione | 337 |
| Example 278 | 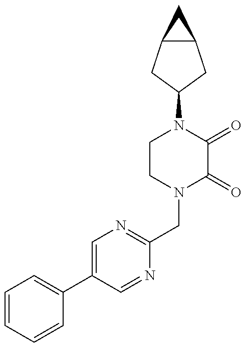 [SMILES: O=C1C(=O)N([C@H]2C[C@H]3C[C@H]3C2)CCN1Cc1ncc(-c2ccccc2)cn1] | 1-((cis)-bicyclo[3.1.0]hexan-3-yl)- 4-((5-phenylpyrimidin-2- yl)methyl)piperazine-2,3-dione | 363 |
| Example 279 | 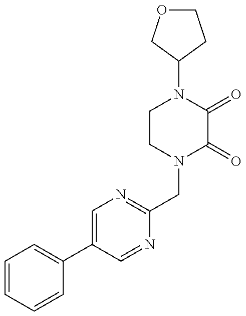 [SMILES: O=C1C(=O)N(C2CCOC2)CCN1Cc1ncc(-c2ccccc2)cn1] | 1-((5-phenylpyrimidin-2- yl)methyl)-4-(tetrahydrofuran-3- yl)piperazine-2,3-dione | 353 |
| Example 280 | 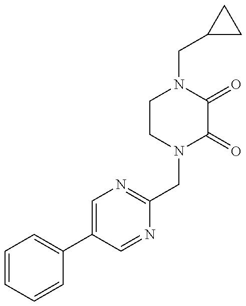 [SMILES: O=C1C(=O)N(CC2CC2)CCN1Cc1ncc(-c2ccccc2)cn1] | 1-(cyclopropylmethyl)-4-((5- phenylpyrimidin-2- yl)methyl)piperazine-2,3-dione | 337 |
| Example 281 | 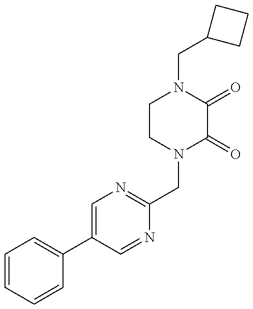 [SMILES: O=C1C(=O)N(CC2CCC2)CCN1Cc1ncc(-c2ccccc2)cn1] | 1-(cyclobutylmethyl)-4-((5- phenylpyrimidin-2- yl)methyl)piperazine-2,3-dione | 351 |
| Example 282 | 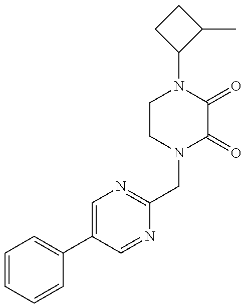 [SMILES: CC1CCC1N1CCN(Cc2ncc(-c3ccccc3)cn2)C(=O)C1=O] | 1-(2-methylcyclobutyl)-4-((5- phenylpyrimidin-2- yl)methyl)piperazine-2,3-dione | 351 |
| Example 283 | 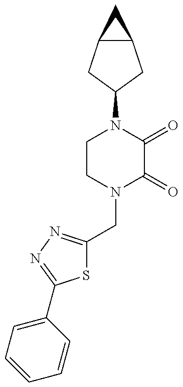 [SMILES: O=C1C(=O)N([C@H]2C[C@H]3C[C@H]3C2)CCN1Cc1nnc(-c2ccccc2)s1] | 1-((cis)-bicyclo[3.1.0]hexan-3-yl)- 4-((5-phenyl-1,3,4-thiadiazol-2- yl)methyl)piperazine-2,3-dione | 369 |
| Example 284 | 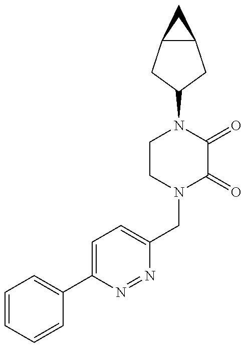 | 1-((cis)-bicyclo[3.1.0]hexan-3-yl)- 4-((6-phenylpyridazin-3- yl)methyl)piperazine-2,3-dione | 363 |
| Example 285 | 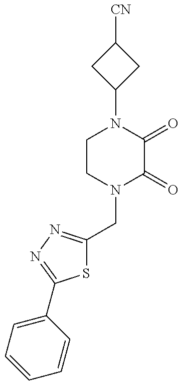 [SMILES: N#CC1CC(N2CCN(Cc3nnc(-c4ccccc4)s3)C(=O)C2=O)C1] | 3-(2,3-dioxo-4-((5-phenyl-1,3,4- thiadiazol-2-yl)methyl)piperazin- 1-yl)cyclobutane-1-carbonitrile | 368 |
| Example 286 | 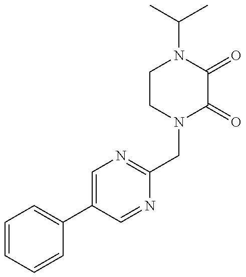 [SMILES: CC(C)N1CCN(Cc2ncc(-c3ccccc3)cn2)C(=O)C1=O] | 1-isopropyl-4-((5- phenylpyrimidin-2- yl)methyl)piperazine-2,3-dione | 325 |
| Example 287 | 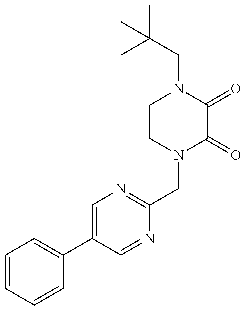 [SMILES: CC(C)(C)CN1CCN(Cc2ncc(-c3ccccc3)cn2)C(=O)C1=O] | 1-neopentyl-4-((5- phenylpyrimidin-2- yl)methyl)piperazine-2,3-dione | 353 |
| Example 288 | 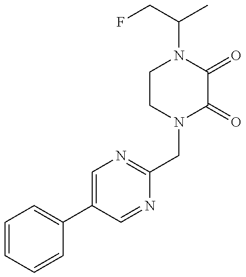 [SMILES: CC(CF)N1CCN(Cc2ncc(-c3ccccc3)cn2)C(=O)C1=O] | 1-(1-fluoropropan-2-yl)-4-((5- phenylpyrimidin-2- yl)methyl)piperazine-2,3-dione | 343 |
| Example 289 | 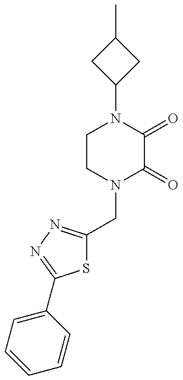 [SMILES: CC1CC(N2CCN(Cc3nnc(-c4ccccc4)s3)C(=O)C2=O)C1] | 1-(3-methylcyclobutyl)-4-((5- phenyl-1,3,4-thiadiazol-2- yl)methyl)piperazine-2,3-dione | 356 |
| Example 290 | 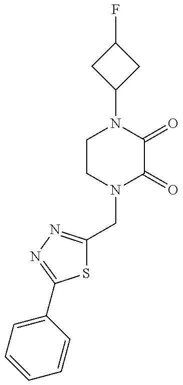 [SMILES: O=C1C(=O)N(C2CC(F)C2)CCN1Cc1nnc(-c2ccccc2)s1] | 1-(3-fluorocyclobutyl)-4-((5- phenyl-1,3,4-thiadiazol-2- yl)methyl)piperazine-2,3-dione | 361 |
| Example 291 | 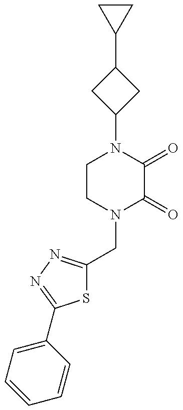 [SMILES: O=C1C(=O)N(C2CC(C3CC3)C2)CCN1Cc1nnc(-c2ccccc2)s1] | 1-(3-cyclopropylcyclobutyl)-4-((5- phenyl-1,3,4-thiadiazol-2- yl)methyl)piperazine-2,3-dione | 383 |
| Example 292 | 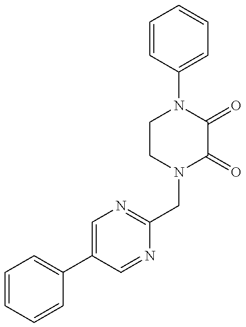 [SMILES: O=C1C(=O)N(c2ccccc2)CCN1Cc1ncc(-c2ccccc2)cn1] | 1-phenyl-4-((5-phenylpyrimidin-2- yl)methyl)piperazine-2,3-dione | 359 |
| Example 293 | 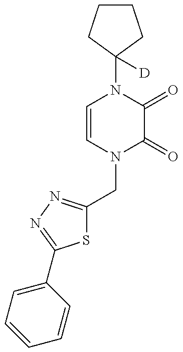 [SMILES: [2H]C1(n2ccn(Cc3nnc(-c4ccccc4)s3)c(=O)c2=O)CCCC1] | 1-(cyclopentyl-1-d)-4-((5-phenyl- 1,3,4-thiadiazol-2-yl)methyl)-1,4- dihydropyrazine-2,3-dione | 356 |
| Example 294 | 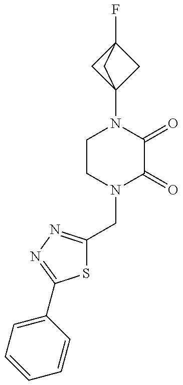 [SMILES: O=C1C(=O)N(C23CC(F)(C2)C3)CCN1Cc1nnc(-c2ccccc2)s1] | 1-(3-fluorobicyclo[1.1.1]pentan-1- yl)-4-((5-phenyl-1,3,4-thiadiazol- 2-yl)methyl)piperazine-2,3-dione | 373 |
| Example 295 | 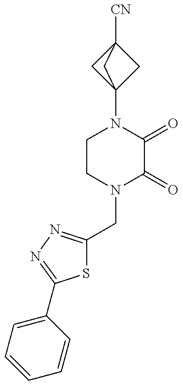 [SMILES: N#CC12CC(N3CCN(Cc4nnc(-c5ccccc5)s4)C(=O)C3=O)(C1)C2] | 3-(2,3-dioxo-4-((5-phenyl-1,3,4- thiadiazol-2-yl)methyl)piperazin- 1-yl)bicyclo[1.1.1]pentane-1- carbonitrile | 380 |
| Example 296 | 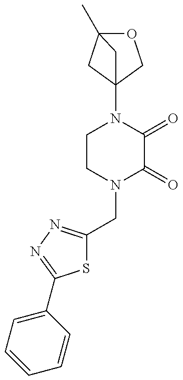 [SMILES: CC12CC(N3CCN(Cc4nnc(-c5ccccc5)s4)C(=O)C3=O)(CO1)C2] | 1-(1-methyl-2- oxabicyclo[2.1.1]hexan-4-yl)-4- ((5-phenyl-1,3,4-thiadiazol-2- yl)methyl)piperazine-2,3-dione | 385 |
| Example 297 | 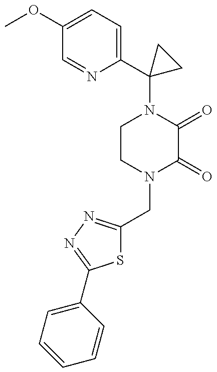 [SMILES: COc1ccc(C2(N3CCN(Cc4nnc(-c5ccccc5)s4)C(=O)C3=O)CC2)nc1] | 1-(1-(5-methoxypyridin-2- yl)cyclopropyl)-4-((5-phenyl- 1,3,4-thiadiazol-2- yl)methyl)piperazine-2,3-dione | 436 |
| Example 298 | 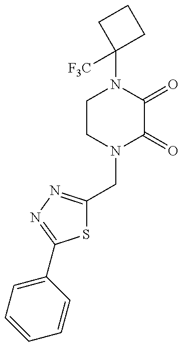 [SMILES: O=C1C(=O)N(C2(C(F)(F)F)CCC2)CCN1Cc1nnc(-c2ccccc2)s1] | 1-((5-phenyl-1,3,4-thiadiazol-2- yl)methyl)-4-(1- (trifluoromethyl)cyclobutyl) piperazine-2,3-dione | 411 |
| Example 299 | 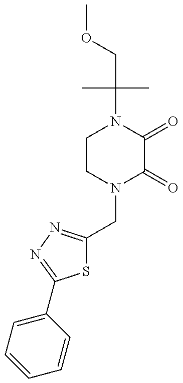 [SMILES: COCC(C)(C)N1CCN(Cc2nnc(-c3ccccc3)s2)C(=O)C1=O] | 1-(1-methoxy-2-methylpropan-2- yl)-4-((5-phenyl-1,3,4-thiadiazol- 2-yl)methyl)piperazine-2,3-dione | 375 |
| Example 300 | 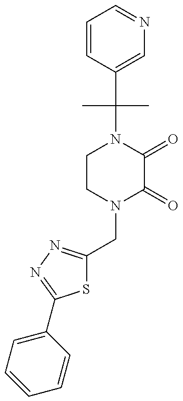 [SMILES: CC(C)(c1cccnc1)N1CCN(Cc2nnc(-c3ccccc3)s2)C(=O)C1=O] | 1-((5-phenyl-1,3,4-thiadiazol-2- yl)methyl)-4-(2-(pyridin-3- yl)propan-2-yl)piperazine-2,3- dione | 408 |
| Example 301 | 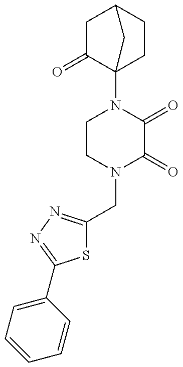 [SMILES: O=C1C(=O)N(C23CCC(CC2=O)C3)CCN1Cc1nnc(-c2ccccc2)s1] | 1-(2-oxobicyclo[2.2.1]heptan-1- yl)-4-((5-phenyl-1,3,4-thiadiazol- 2-yl)methyl)piperazine-2,3-dione | 397 |
| Example 302 | 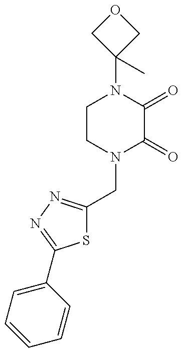 [SMILES: CC1(N2CCN(Cc3nnc(-c4ccccc4)s3)C(=O)C2=O)COC1] | 1-(3-methyloxetan-3-yl)-4-((5- phenyl-1,3,4-thiadiazol-2- yl)methyl)piperazine-2,3-dione | 359 |
| Example 303 | 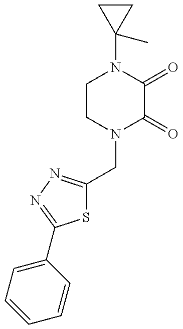 [SMILES: CC1(N2CCN(Cc3nnc(-c4ccccc4)s3)C(=O)C2=O)CC1] | 1-(1-methylcyclopropyl)-4-((5- phenyl-1,3,4-thiadiazol-2- yl)methyl)piperazine-2,3-dione | 343 |
| Example 304 | 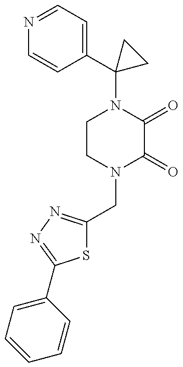 [SMILES: O=C1C(=O)N(C2(c3ccncc3)CC2)CCN1Cc1nnc(-c2ccccc2)s1] | 1-((5-phenyl-1,3,4-thiadiazol-2- yl)methyl)-4-(1-(pyridin-4- yl)cyclopropyl)piperazine-2,3- dione | 406 |
| Example 305 | 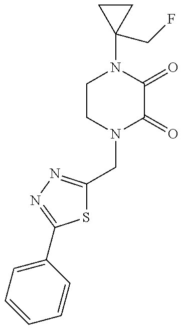 [SMILES: O=C1C(=O)N(C2(CF)CC2)CCN1Cc1nnc(-c2ccccc2)s1] | 1-(1-(fluoromethyl)cyclopropyl)- 4-((5-phenyl-1,3,4-thiadiazol-2- yl)methyl)piperazine-2,3-dione | 361 |
| Example 306 | 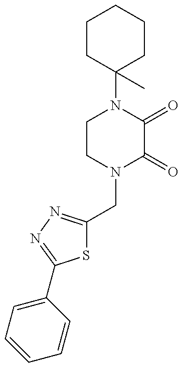 [SMILES: CC1(N2CCN(Cc3nnc(-c4ccccc4)s3)C(=O)C2=O)CCCCC1] | 1-(1-methylcyclohexyl)-4-((5- phenyl-1,3,4-thiadiazol-2- yl)methyl)piperazine-2,3-dione | 385 |
| Example 307 | 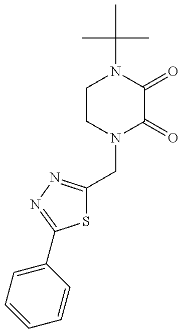 [SMILES: CC(C)(C)N1CCN(Cc2nnc(-c3ccccc3)s2)C(=O)C1=O] | 1-(tert-butyl)-4-((5-phenyl-1,3,4- thiadiazol-2-yl)methyl)piperazine- 2,3-dione | 345 |
| Example 308 | 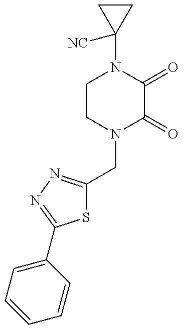 [SMILES: N#CC1(N2CCN(Cc3nnc(-c4ccccc4)s3)C(=O)C2=O)CC1] | 1-(2,3-dioxo-4-((5-phenyl-1,3,4- thiadiazol-2-yl)methyl)piperazin- 1-yl)cyclopropane-1-carbonitrile | 354 |

### Example 309

### Preparation of 1-cyclopentyl-4-((5-(piperidin-1-yl)-1,3,4-thiadiazol-2-yl)methyl)piperazine-2,3-dione

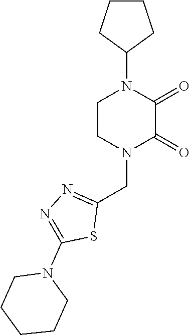 [SMILES: O=C1C(=O)N(C2CCCC2)CCN1Cc1nnc(N2CCCCC2)s1]

Piperidine (6 μL, 0.06 mmol) and Hunig's base (17 uL, 0.10 mmol) were added to a mixture of 1-((5-chloro-1,3,4-thiadiazol-2-yl)methyl)-4-cyclopentylpiperazine-2,3-dione (15 mg, 0.048 mmol) in DMF (95 μL) at room temperature. The reaction mixture was stirred at room temperature for 18 hours. The mixture was purified by reverse phase HPLC (eluting acetonitrile in water, with TFA modifier) to afford 1-cyclopentyl-4-((5-(piperidin-1-yl)-1,3,4-thiadiazol-2-yl)methyl)piperazine-2,3-dione as a TFA salt. ¹H NMR (500 MHz, DMSO-d₆) δ 4.74 (s, 2H), 4.67 (p, J=8.3 Hz, 1H), 3.55 (dd, J=7.0, 4.3 Hz, 2H), 3.50-3.39 (m, 6H), 1.83-1.45 (in, 14H). LC/MS (m/z): 364 (M+H)⁺

Examples shown in Example Table 6 below, were or may be prepared according to procedures analogous to those outlined in Example 309 above using the appropriate starting materials, described in the Preparations or Intermediates above, or as obtained from commercial

Example Table 6

| | | | Mass |
|---|---|---|---|
| Example | Structure | Name | [M + H]⁺ |
| Example 310 | 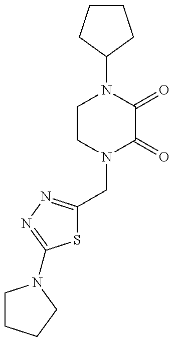 [SMILES: O=C1C(=O)N(C2CCCC2)CCN1Cc1nnc(N2CCCC2)s1] | 1-cyclopentyl-4-((5-(pyrrolidin-1- yl)-1,3,4-thiadiazol-2- yl)methyl)piperazine-2,3-dione | 350 |
| Example 311 | 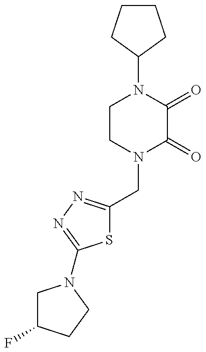 [SMILES: O=C1C(=O)N(C2CCCC2)CCN1Cc1nnc(N2CC[C@H](F)C2)s1] | (S)-1-cyclopentyl-4-((5-(3- fluoropyrrolidin-1-yl)-1,3,4- thiadiazol-2-yl)methyl)piperazine- 2,3-dione | 368 |
| Example 312 | 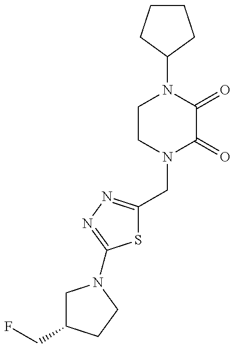 [SMILES: O=C1C(=O)N(C2CCCC2)CCN1Cc1nnc(N2CC[C@H](CF)C2)s1] | (S)-1-cyclopentyl-4-((5-(3- (fluoromethyl)pyrrolidin-1-yl)- 1,3,4-thiadiazol-2- yl)methyl)piperazine-2,3-dione | 382 |
| Example 313 | 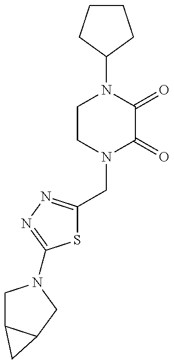 [SMILES: O=C1C(=O)N(C2CCCC2)CCN1Cc1nnc(N2CC3CC3C2)s1] | 1-((5-(3-azabicyclo[3.1.0]hexan-3- yl)-1,3,4-thiadiazol-2-yl)methyl)- 4-cyclopentylpiperazine-2,3-dione | 362 |

### Example 314

### Preparation of 1-cyclopentyl-4-((6-(pyrrolidin-1-yl)pyridazin-3-yl)methyl)piperazine-2,3-dione

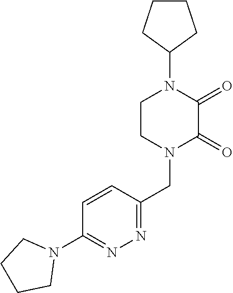 [SMILES: O=C1C(=O)N(C2CCCC2)CCN1Cc1ccc(N2CCCC2)nn1]

Triethylamine (0.032 ml, 0.23 mmol) and pyrrolidine (16 mg, 0.23 mmol) were added to a mixture of 1-((6-chloropyridazin-3-yl)methyl)-4-cyclopentylpiperazine-2,3-dione (0.070 g, 0.23 mmol) in EtOH (2 mL) at room temperature. The reaction mixture was stirred and heated to 100° C. for 12 hours. The reaction mixture was cooled to room temperature and directly purified by reverse phase HPLC (eluting acetonitrile in water, with TFA modifier) to afford 1-cyclopentyl-4-((6-(pyrrolidin-1-yl)pyridazin-3-yl)methyl)piperazine-2,3-dione. ¹H NMR (500 MHz, methanol-d₄) δ=7.80 (d, J=9.8 Hz, 1H), 7.68 (d, J=9.8 Hz, 1H), 4.85-4.75 (m, 1H), 4.78 (s, 2H), 3.75-3.52 (m, 8H), 2.20-2.08 (m, 4H), 2.00-1.85 (m, 2H), 1.83-1.72 (m, 2H), 1.70-1.56 (m, 4H). LC/MS (m/z): 344 (M+H)⁺

Examples shown in Example Table 7 below, were or may be prepared according to procedures analogous to those outlined in Example 314 above using the appropriate starting materials, described in the Preparations or Intermediates above, or as obtained from commercial sources.

Example Table 7

| | | | Mass |
|---|---|---|---|
| Example | Structure | Name | [M + H]⁺ |
| Example 315 | 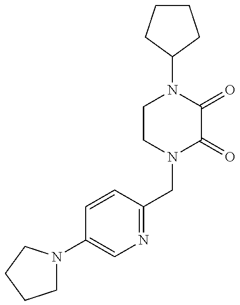 [SMILES: O=C1C(=O)N(C2CCCC2)CCN1Cc1ccc(N2CCCC2)cn1] | 1-cyclopentyl-4-((6-(pyrrolidin-1- yl)pyridin-3-yl)methyl)piperazine- 2,3-dione | 343 |
| Example 316 | 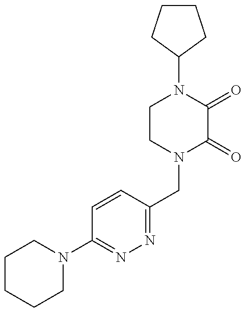 [SMILES: O=C1C(=O)N(C2CCCC2)CCN1Cc1ccc(N2CCCCC2)nn1] | 1-cyclopentyl-4-((6-(piperidin-1- yl)pyridazin-3-yl)methyl)piperazine- 2,3-dione | 358 |
| Example 317 | 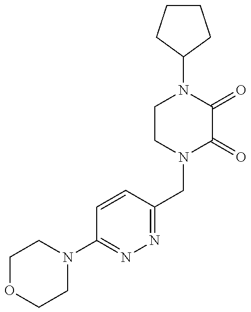 [SMILES: O=C1C(=O)N(C2CCCC2)CCN1Cc1ccc(N2CCOCC2)nn1] | 1-cyclopentyl-4-((6- morpholinopyridazin-3- yl)methyl)piperazine-2,3-dione | 360 |
| Example 318 | 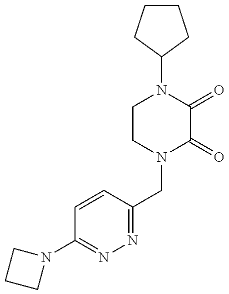 [SMILES: O=C1C(=O)N(C2CCCC2)CCN1Cc1ccc(N2CCC2)nn1] | 1-((6-(azetidin-1-yl)pyridazin-3- yl)methyl)-4-cyclopentylpiperazine- 2,3-dione | 330 |
| Example 319 | 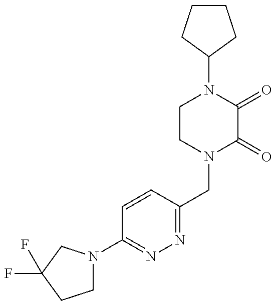 [SMILES: O=C1C(=O)N(C2CCCC2)CCN1Cc1ccc(N2CCC(F)(F)C2)nn1] | 1-cyclopentyl-4-((6-(3,3- difluoropyrrolidin-1-yl)pyridazin-3- yl)methyl)piperazine-2,3-dione | 380 |
| Example 320 | 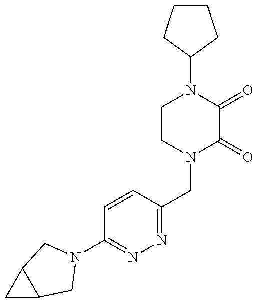 [SMILES: O=C1C(=O)N(C2CCCC2)CCN1Cc1ccc(N2CC3CC3C2)nn1] | 1-((6-(3-azabicyclo[3.1.0]hexan-3- yl)pyridazin-3-yl)methyl)-4- cyclopentylpiperazine-2,3-dione | 356 |

### Example 321

### Preparation of 1-((6-(1H-1,2,3-triazol-1-yl)pyridazin-3-yl)methyl)-4-cyclopentylpiperazine-2,3-dione

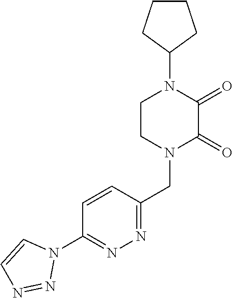 [SMILES: O=C1C(=O)N(C2CCCC2)CCN1Cc1ccc(-n2ccnn2)nn1]

A mixture of 1-((6-chloropyridazin-3-yl)methyl)-4-cyclopentylpiperazine-2,3-dione (0.030 g, 0.097 mmol), cesium carbonate (63 mg, 0.19 mmol), and 2H-1,2,3-triazole (7 μl, 0.1 mmol) in DMF (1 mL) was stirred and heated at 80° C. for 72 hours. The mixture was cooled to room temperature and diluted with DCM (5 mL). The mixture was filtered through Celite, and the filtrate was concentrated under reduced pressure. The residue was purified by silica gel chromatography (eluting [1:3 EtOH:ethyl acetate] in hexanes) followed by basic alumina chromatography (eluting [1:3 EtOH:ethyl acetate] in hexanes) to afford a 1:1 mixture of 1H- and 2-H triazole isomers. The isomeric mixture was resolved by chiral SFC purification (OJ-H 21×250 mm column, 35% MeOH w/ 0.1% NH₄OH as cosolvent) to afford 1-((6-(1H-1,2,3-triazol-1-yl)pyridazin-3-yl)methyl)-4-cyclopentylpiperazine-2,3-dione as the first eluting peak. ¹H NMR (500 MHz, DMSO-d₆) δ 9.10 (d, J=1.1 Hz, 1H), 8.44 (d, J=9.0 Hz, 1H), 8.10 (d, J=1.1 Hz, 1H), 8.02 (d, J=9.0 Hz, 1H), 4.96 (s, 2H), 4.78-4.64 (m, 1H), 3.70-3.66 (m, 2H), 3.54-3.51 (m, 2H), 1.82-1.74 (m, 2H), 1.70-1.64 (m, 2H), 1.60-1.52 (m, 4H). LC/MS (m/z): 342 (M+H)⁺

Examples shown in Example Table 8 below, were or may be prepared according to procedures analogous to those outlined in Example 321 above using the appropriate starting materials, described in the Preparations or Intermediates above, or as obtained from commercial sources.

Example Table 8

| | | | Mass |
|---|---|---|---|
| Example | Structure | Name | [M + H]⁺ |
| Example 322 | 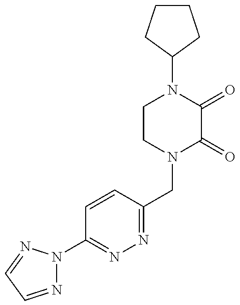 [SMILES: O=C1C(=O)N(C2CCCC2)CCN1Cc1ccc(-n2nccn2)nn1] | 1-((6-(2H-1,2,3-triazol-2- yl)pyridazin-3-yl)methyl)-4- cyclopentylpiperazine-2,3-dione | 342 |
| Example 323 | 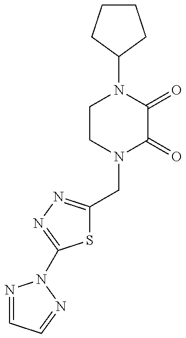 [SMILES: O=C1C(=O)N(C2CCCC2)CCN1Cc1nnc(-n2nccn2)s1] | 1-((5-(2H-1,2,3-triazol-2-yl)-1,3,4- thiadiazol-2-yl)methyl)-4- cyclopentylpiperazine-2,3-dione | 348 |
| Example 324 | 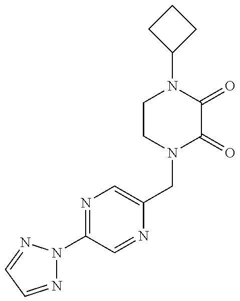 [SMILES: O=C1C(=O)N(C2CCC2)CCN1Cc1cnc(-n2nccn2)cn1] | 1-((5-(2H-1,2,3-triazol-2- yl)pyrazin-2-yl)methyl)-4- cyclobutylpiperazine-2,3-dione | 328 |
| Example 325 | 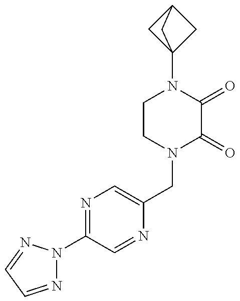 [SMILES: O=C1C(=O)N(C23CC(C2)C3)CCN1Cc1cnc(-n2nccn2)cn1] | 1-((5-(2H-1,2,3-triazol-2- yl)pyrazin-2-yl)methyl)-4- (bicyclo[1.1.1]pentan-1- yl)piperazine-2,3-dione | 340 |
| Example 326 | 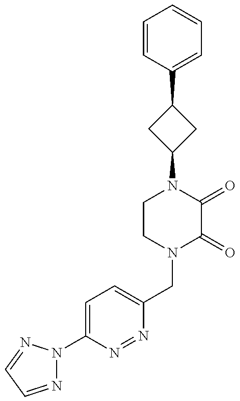 [SMILES: O=C1C(=O)N([C@H]2C[C@@H](c3ccccc3)C2)CCN1Cc1ccc(-n2nccn2)nn1] | 1-((6-(2H-1,2,3-triazol-2- yl)pyridazin-3-yl)methyl)-4-((cis)- 3-phenylcyclobutyl)piperazine- 2,3-dione | 404 |
| Example 327 | 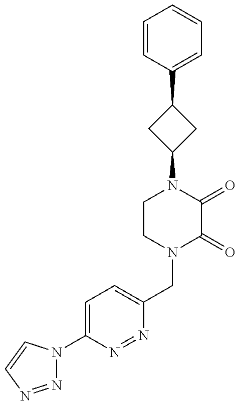 [SMILES: O=C1C(=O)N([C@H]2C[C@@H](c3ccccc3)C2)CCN1Cc1ccc(-n2ccnn2)nn1] | 1-((6-(1H-1,2,3-triazol-1- yl)pyridazin-3-yl)methyl)-4-((cis)- 3-phenylcyclobutyl)piperazine- 2,3-dione | 404 |
| Example 328 | 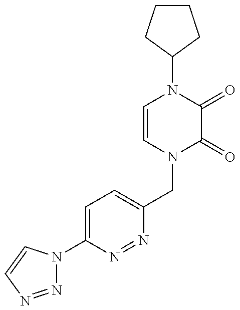 [SMILES: O=c1c(=O)n(C2CCCC2)ccn1Cc1ccc(-n2ccnn2)nn1] | 1-((6-(1H-1,2,3-triazol-1- yl)pyridazin-3-yl)methyl)-4- cyclopentyl-1,4-dihydropyrazine- 2,3-dione | 340 |
| Example 329 | 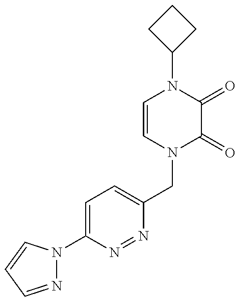 [SMILES: O=c1c(=O)n(C2CCC2)ccn1Cc1ccc(-n2cccn2)nn1] | 1-((6-(1H-pyrazol-1-yl)pyridazin- 3-yl)methyl)-4-cyclobutyl-1,4- dihydropyrazine-2,3-dione | 325 |
| Example 330 | 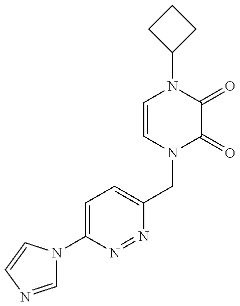 [SMILES: O=c1c(=O)n(C2CCC2)ccn1Cc1ccc(-n2ccnc2)nn1] | 1-((6-(1H-imidazol-1- yl)pyridazin-3-yl)methyl)-4- cyclobutyl-1,4-dihydropyrazine- 2,3-dione | 325 |
| Example 331 | 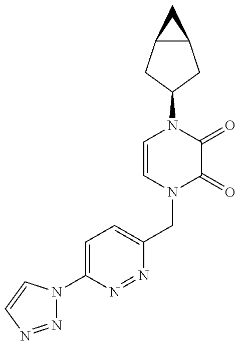 [SMILES: O=c1c(=O)n([C@H]2C[C@H]3C[C@H]3C2)ccn1Cc1ccc(-n2ccnn2)nn1] | 1-((6-(1H-1,2,3-triazol-1- yl)pyridazin-3-yl)methyl)-4-((cis)- bicyclo[3.1.0]hexan-3-yl)-1,4- dihydropyrazine-2,3-dione | 352 |
| Example 332 | 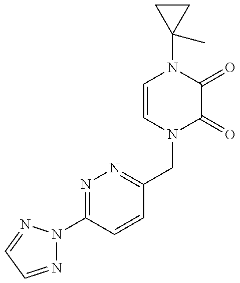 [SMILES: CC1(n2ccn(Cc3ccc(-n4nccn4)nn3)c(=O)c2=O)CC1] | 1-((6-(2H-1,2,3-triazol-2- yl)pyridazin-3-yl)methyl)-4-(1- methylcyclopropyl)-1,4- dihydropyrazine-2,3-dione | 326 |
| Example 333 | 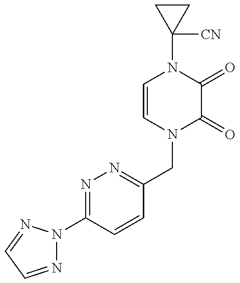 [SMILES: N#CC1(n2ccn(Cc3ccc(-n4nccn4)nn3)c(=O)c2=O)CC1] | 1-(4-((6-(2H-1,2,3-triazol-2- yl)pyridazin-3-yl)methyl)-2,3- dioxo-3,4-dihydropyrazin-1(2H)- yl)cyclopropane-1-carbonitrile | 337 |
| Example 334 | 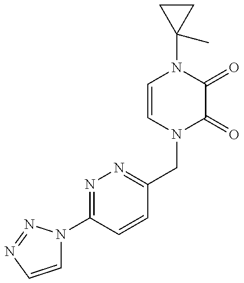 [SMILES: CC1(n2ccn(Cc3ccc(-n4ccnn4)nn3)c(=O)c2=O)CC1] | 1-((6-(1H-1,2,3-triazol-1- yl)pyridazin-3-yl)methyl)-4-(1- methylcyclopropyl)-1,4- dihydropyrazine-2,3-dione | 326 |
| Example 335 | 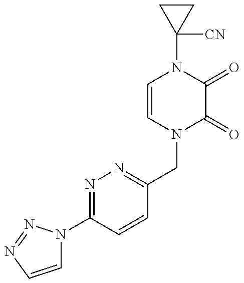 [SMILES: N#CC1(n2ccn(Cc3ccc(-n4ccnn4)nn3)c(=O)c2=O)CC1] | 1-(4-((6-(1H-1,2,3-triazol-1- yl)pyridazin-3-yl)methyl)-2,3- dioxo-3,4-dihydropyrazin-1(2H)- yl)cyclopropane-1-carbonitrile | 337 |

### Example 336

### Preparation of 1-((6-(2H-1,2,3-triazol-2-yl)pyridin-3-yl)methyl)-4-cyclopentylpiperazine-2,3-dione

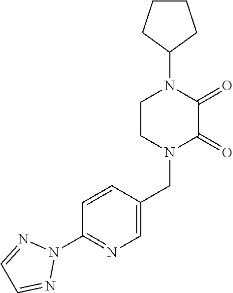 [SMILES: O=C1C(=O)N(C2CCCC2)CCN1Cc1ccc(-n2nccn2)nc1]

A mixture of 1-((6-chloropyridin-3-yl)methyl)-4-cyclopentylpiperazine-2,3-dione (0.100 g, 0.325 mmol), cesium carbonate (212 mg, 0.650 mmol), and 2H-1,2,3-triazole (0.023 ml, 0.39 mmol) in DMF (1.0 mL) and N-methyl-2-pyrrolidone (1.0 mL) was stirred and heated at 200° C. in a microwave reaction for 1 hour. The mixture was cooled to room temperature and filtered. The filtrate was purified by reverse phase HPLC (eluting acetonitrile in water, with TFA modifier) to afford 1-((6-(2H-1,2,3-triazol-2-yl)pyridin-3-yl)methyl)-4-cyclopentylpiperazine-2,3-dione. ¹H NMR (500 MHz, DMSO-d₆) δ 8.54 (s, 1H), 8.18 (s, 2H), 8.01-8.00 (m, 2H), 4.71-4.65 (m, 1H), 4.68 (s, 2H), 3.56-3.52 (m, 2H), 3.48-3.44 (m, 2H), 1.80-1.71 (m, 2H), 1.68-1.62 (m, 2H), 1.58-1.50 (m, 4H). LC/MS (m/z): 341 (M+H)⁺

### Example 337

### Preparation of 1-cyclopentyl-4-((5-phenylpyrimidin-2-yl)methyl)-1,4-dihydropyrazine-2,3-dione

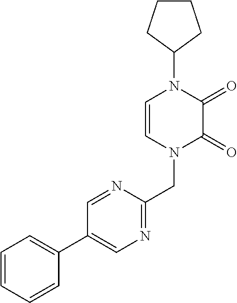 [SMILES: O=c1c(=O)n(C2CCCC2)ccn1Cc1ncc(-c2ccccc2)cn1]

Cyclopentanamine (57 mg, 0.67 mmol) was added to a mixture of ethyl 2-oxo-2-((2-oxoethyl)((5-phenylpyrimidin-2-yl)methyl)amino)acetate (200 mg, 0.6 mmol) in DCE (5 mL) at room temperature. The reaction mixture was stirred at room temperature for 16 hours. The mixture was diluted with water (10 mL) and DCM (5 mL). The aqueous layer was separated and extracted with additional DCM (2×5 mL). The organic layers were combined, dried over anhydrous sodium sulfate, filtered, and concentrated under reduced pressure. The residue was purified by reverse phase HPLC (eluting acetonitrile in water, with TFA modifier) to afford 1-cyclopentyl-4-((5-phenylpyrimidin-2-yl)methyl)-1,4-dihydropyrazine-2,3-dione. ¹H NMR (400 MHz, methanol-d₄) δ 9.00 (s, 2H), 7.73-7.67 (m, 2H), 7.56-7.45 (m, 3H), 6.73 (d, J=6.1 Hz, 1H), 6.66 (d, J=6.4 Hz, 1H), 5.29 (s, 2H), 5.07 (quin, J=7.9 Hz, 1H), 2.21-2.08 (m, 2H), 1.95-1.71 (m, 6H). LC/MS (m/z): 349 (M+H)⁺

Examples shown in Example Table 9 below, were or may be prepared according to procedures analogous to those outlined in Example 337 above using the appropriate starting materials, described in the Preparations or Intermediates above, or as obtained from commercial sources.

Example Table 9

| Example | Structure | Name | Mass [M + H]⁺ |
|---|---|---|---|
| Example 338 | 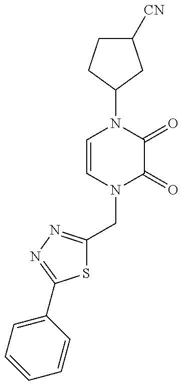 [SMILES: N#CC1CCC(n2ccn(Cc3nnc(-c4ccccc4)s3)c(=O)c2=O)C1] | 3-(2,3-dioxo-4-((5-phenyl- 1,3,4-thiadiazol-2- yl)methyl)-3,4- dihydropyrazin-1(2H)- yl)cyclopentane-1- carbonitrile | 380 |

### Example 339

### Preparation of 1-cyclopentyl-4-((6-propoxypyridazin-3-yl)methyl)piperazine-2,3-dione

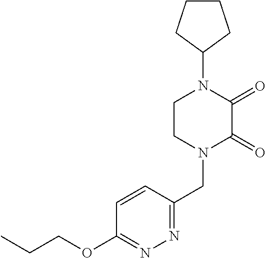 [SMILES: CCCOc1ccc(CN2CCN(C3CCCC3)C(=O)C2=O)nn1]

A mixture of 1-((6-chloropyridazin-3-yl)methyl)-4-cyclopentylpiperazine-2,3-dione (0.100 g, 0.3 mmol), propan-1-ol (58 mg, 0.97 mmol), RockPhos Pd G3 ([(2-Di-tert-butylphosphino-3-methoxy-6-methyl-2′,4′,6′-triisopropyl-1,1′-biphenyl)-2-(2-aminobiphenyl)]palladium(II) methanesulfonateprecatalyst) (3 mg, 3 μmol), and Cs₂CO₃ (211 mg, 0.648 mmol) in DMF (1 mL) was sparged with nitrogen at room temperature. The reaction mixture was stirred and heated to 90° C. for 12 hours. The reaction mixture was cooled to room temperature, filtered, and then directly purified by reverse phase HPLC (eluting acetonitrile in water, with TFA modifier) to afford 1-cyclopentyl-4-((6-propoxypyridazin-3-yl)methyl)piperazine-2,3-dione. ¹H NMR (400 MHz, methanol-d₄) δ 7.68 (d, J=9.4 Hz, 1H), 7.24 (d, J=9.4 Hz, 1H), 4.85 (s, 2H), 4.80 (t, J=8.2 Hz, 1H), 4.40 (t, J=6.7 Hz, 2H), 3.73-3.65 (m, 2H), 3.61-3.54 (m, 2H), 1.95-1.83 (m, 4H), 1.81-1.72 (m, 2H), 1.70-1.54 (m, 4H), 1.05 (t, J=7.4 Hz, 3H). LC/MS (m/z): 333 (M+H)⁺

### Example 340

### Preparation of 1-cyclopentyl-4-((6-ethoxypyridazin-3-yl)methyl)piperazine-2,3-dione

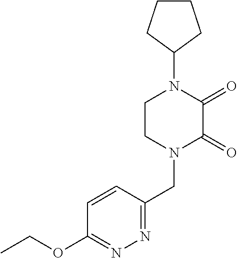 [SMILES: CCOc1ccc(CN2CCN(C3CCCC3)C(=O)C2=O)nn1]

Sodium ethoxide (110 mg, 1.6 mmol) was added to a mixture of 1-((6-chloropyridazin-3-yl)methyl)-4-cyclopentylpiperazine-2,3-dione (0.100 g, 0.32 mmol) in EtOH (10 mL) at room temperature. The reaction mixture was stirred at room temperature for 4 hours. 2 M HCl was added dropwise to the reaction mixture until pH ˜7. The mixture was concentrated under reduced pressure. The residue was purified by reverse phase HPLC (eluting acetonitrile in water, with formic acid modifier) to afford 1-cyclopentyl-4-((6-ethoxypyridazin-3-yl)methyl)piperazine-2,3-dione. ¹H NMR (400 MHz, DMSO-d₆) δ 7.54 (d, J=9.05 Hz, 1H), 7.16 (d, J=9.05 Hz, 1H), 4.72 (s, 2H), 4.62-4.68 (m, 1H), 4.38-4.42 (m, 2H), 3.51-3.58 (m, 2H), 3.37-3.47 (m, 2H), 1.78-1.67 (m, 2H), 1.67-1.57 (m, 2H), 1.57-1.43 (m, 4H), 1.33 (t, J=6.97 Hz, 3H). LC/MS (m/z): 319 (M+H)⁺

### Example 341

### Preparation of (R or S)-4-cyclopentyl-5-methyl-1-((5-phenylpyrimidin-2-yl)methyl)piperazine-2,3-dione

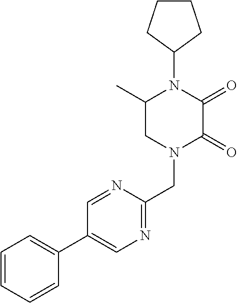 [SMILES: CC1CN(Cc2ncc(-c3ccccc3)cn2)C(=O)C(=O)N1C1CCCC1]

### Step A: ethyl 2-((2-methylallyl)amino)-2-oxoacetate

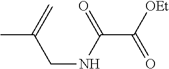

Ethyl 2-chloro-2-oxoacetate (1.6 mL, 14 mmol) was added to a mixture of 2-methylallylamine (1.0 g, 14 mmol) and triethylamine (5.9 mL, 42 mmol) in DCM (15 mL) at 0° C. The reaction mixture was warmed to room temperature and stirred for 15 hours. The reaction mixture was diluted with water (30 mL) and extracted with DCM (3×30 mL). The organic layers were combined, washed with brine (20 mL), dried over anhydrous sodium sulfate, filtered, and concentrated under reduced pressure. The residue was purified by silica gel chromatography (eluting ethyl acetate in petroleum ether) to afford ethyl 2-((2-methylallyl)amino)-2-oxoacetate. ¹H NMR (500 MHz, DMSO-d₆) δ 9.08 (s, 1H), 4.77 (s, 1H), 4.75 (s, 1H), 4.23 (q, J=7.0 Hz, 2H), 3.66 (d, J=6.0 Hz, 2H), 1.66 (s, 3H), 1.27 (t, J=7.2 Hz, 3H). LC/MS (m/z): 172 (M+H)⁺

### Step B: ethyl 2-((2-methylallyl)((5-phenylpyrimidin-2-yl)methyl)amino)-2-oxoacetate

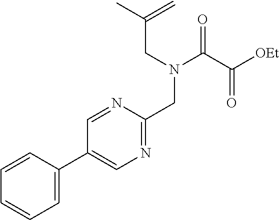 [SMILES: C=C(C)CN(Cc1ncc(-c2ccccc2)cn1)C(=O)C(=O)OCC]

NaH (83 mg, 2 mmol, 60% dispersion in mineral oil) was added to a mixture of ethyl 2-((2-methylallyl)amino)-2-oxoacetate (254 mg, 1.49 mmol) in DMF (5 mL) at 0° C. The reaction mixture was warmed to room temperature and stirred for 30 minutes. A mixture of 2-(bromomethyl)-5-phenylpyrimidine (370 mg, 1.49 mmol) in DMF (2 mL) was added to the reaction mixture at 0° C. The reaction mixture was warmed to room temperature and stirred for an additional 1 hour. The reaction mixture was diluted with water (50 mL) and extracted with ethyl acetate (3×20 mL). The organic layers were combined, washed with brine (20 mL), dried over anhydrous sodium sulfate, filtered, and concentrated under reduced pressure. The residue was purified by silica gel chromatography (eluting ethyl acetate in petroleum ether) to afford ethyl 2-((2-methylallyl)((5-phenylpyrimidin-2-yl)methyl)amino)-2-oxoacetate. LC/MS (m/z): 340 (M+H)⁺

### Step C: ethyl 2-oxo-2-((2-oxopropyl)((5-phenylpyrimidin-2-yl)methyl)amino)acetate

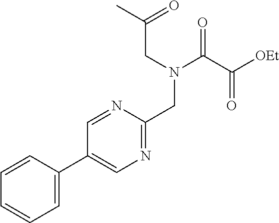 [SMILES: CCOC(=O)C(=O)N(CC(C)=O)Cc1ncc(-c2ccccc2)cn1]

A mixture of ethyl 2-((2-methylallyl)((5-phenylpyrimidin-2-yl)methyl)amino)-2-oxoacetate (163 mg, 0.48 mmol), 2,6-dimethylpyridine (0.11 mL, 0.96 mmol), potassium osmate(VI) dihydrate (35 mg, 0.095 mmol), and sodium periodate (411 mg, 1.92 mmol) in 1,4-dioxane (4 mL) and water (1 mL) was stirred at room temperature for 15 hours. The reaction mixture was quenched with saturated aqueous Na₂SO₃ (5 mL) and extracted with ethyl acetate (3×20 mL). The organic layers were combined, washed with brine (20 mL), dried over anhydrous sodium sulfate, filtered, and concentrated under reduced pressure. The residue was purified by silica gel chromatography (eluting ethyl acetate in petroleum ether) to afford ethyl 2-oxo-2-((2-oxopropyl)((5-phenylpyrimidin-2-yl)methyl)amino)acetate. LC/MS (m/z): 342 (M+H)⁺

### Step D: (R or S)-4-cyclopentyl-5-methyl-1-((5-phenylpyrimidin-2-yl)methyl)piperazine-2,3-dione

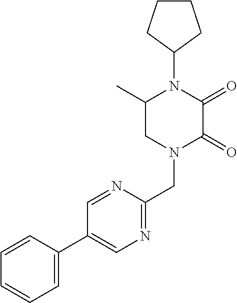 [SMILES: CC1CN(Cc2ncc(-c3ccccc3)cn2)C(=O)C(=O)N1C1CCCC1]

Sodium triacetoxyborohydride (75 mg, 0.45 mmol) was added to a mixture of ethyl 2-oxo-2-((2-oxopropyl)((5-phenylpyrimidin-2-yl)methyl)amino)acetate (0.040 g, 0.12 mmol) and cyclopentanamine (10 mg, 0.1 mmol) in DCE (1.5 mL) at room temperature. The reaction mixture was stirred at room temperature for 12 hours. The reaction mixture was quenched with water (1 mL) and then concentrated under reduced pressure. The residue was purified by reverse phase HPLC (eluting acetonitrile in water, with TFA modifier) to afford 4-cyclopentyl-5-methyl-1-((5-phenylpyrimidin-2-yl)methyl)piperazine-2,3-dione as a mixture of R and S enantiomers. The racemic mixture was resolved by chiral-SFC (eluting ammonia in methanol in CO₂) to afford (R or S)-4-cyclopentyl-5-methyl-1-((5-phenylpyrimidin-2-yl)methyl)piperazine-2,3-dione as the second eluting peak. ¹H NMR (500 MHz, methanol-d₄) δ 9.02 (s, 2H), 7.72-7.68 (m, 2H), 7.56-7.50 (m, 2H), 7.49-7.43 (m, 1H), 5.07-5.00 (m, 1H), 4.87-4.83 (m, 1H), 4.44-4.34 (m, 1H), 4.18-4.12 (m, 1H), 3.90-3.84 (m, 1H), 3.49-3.44 (m, 1H), 2.03-1.90 (m, 2H), 1.90-1.78 (m, 3H), 1.76-1.58 (m, 3H), 1.48 (d, J=6.5 Hz, 3H). LC/MS (m/z): 365 (M+H)⁺

### Example 342

### Preparation of 1-cyclopentyl-4-((5-(pyridin-2-yl)pyrimidin-2-yl)methyl)piperazine-2,3-dione

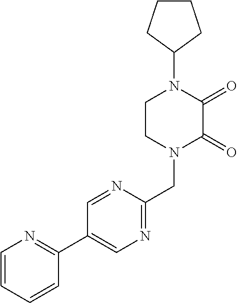 [SMILES: O=C1C(=O)N(C2CCCC2)CCN1Cc1ncc(-c2ccccn2)cn1]

A mixture of 1-cyclopentyl-4-((5-(4,4,5,5-tetramethyl-1,3,2-dioxaborolan-2-yl)pyrimidin-2-yl)methyl)piperazine-2,3-dione (20 mg, 50 μmol), 2-bromopyridine (10 mg, 60 μmol), potassium carbonate (21 mg, 150 μmol), and [1,1′-bis(diphenylphosphino)ferrocene]-dichloropalladium(II) (2 mg, 3 μmol) in water (0.25 mL) and 1,4-dioxane (1.0 mL) was sparged with nitrogen at room temperature. The reaction mixture was stirred and heated at 80° C. for 16 hours. The reaction mixture was cooled to room temperature and concentrated under reduced pressure. The residue was diluted with water (1 mL) and washed with ethyl acetate (3×3 mL). The organic layers were combined, washed with additional water (2×1 mL), dried over anhydrous sodium sulfate, filtered, and concentrated under reduced pressure. The residue was purified by reverse phase HPLC (eluting acetonitrile in water, with TFA modifier) to afford 1-cyclopentyl-4-((5-(pyridin-2-yl)pyrimidin-2-yl)methyl)piperazine-2,3-dione. LC/MS (m/z): 352 (M+H)⁺

Examples shown in Example Table 10 below, were or may be prepared according to procedures analogous to those outlined in Example 342 above using the appropriate starting materials, described in the Preparations or Intermediates above, or as obtained from commercial sources.

Example Table 10

| Example | Structure | Name | Mass [M + H]⁺ |
|---|---|---|---|
| Example 343 | 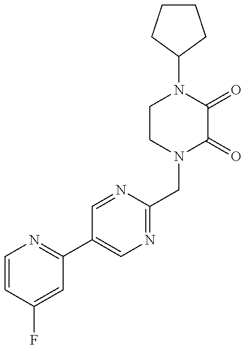 [SMILES: O=C1C(=O)N(C2CCCC2)CCN1Cc1ncc(-c2cc(F)ccn2)cn1] | 1-cyclopentyl-4-((5-(4- fluoropyridin-2-yl)pyrimidin-2- yl)methyl)piperazine-2,3-dione | 370 |

### Example 344

### Preparation of (R or S, R or S)-1-(3-methylcyclopentyl)-4-((5-phenylpyrimidin-2-yl)methyl)piperazine-2,3-dione

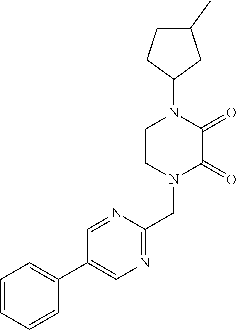 [SMILES: CC1CCC(N2CCN(Cc3ncc(-c4ccccc4)cn3)C(=O)C2=O)C1]

Sodium triacetoxyborohydride (285 mg, 1.34 mmol) was added to a mixture of ethyl 2-oxo-2-((2-oxoethyl)((5-phenylpyrimidin-2-yl)methyl)amino)acetate (220 mg, 0.67 mmol) and 3-methylcyclopentan-1-amine (133 mg, 1.34 mmol) in DCE (1 mL) at room temperature. The reaction mixture was stirred at room temperature for 12 hours. The mixture was quenched with water and extracted with ethyl acetate (3×). The organic layers were combined, washed with brine, dried over anhydrous sodium sulfate, filtered, and concentrated under reduced pressure. The residue was purified by reverse phase HPLC (eluting acetonitrile in water, with ammonium bicarbonate modifier) to afford 1-(3-methylcyclopentyl)-4-((5-phenylpyrimidin-2-yl)methyl)piperazine-2,3-dione as a mixture of R and S isomers. The racemic mixture was resolved by chiral SFC (eluting ammonia in methanol in CO₂) to afford (R or S, R or S)-1-(3-methylcyclopentyl)-4-((5-phenylpyrimidin-2-yl)methyl)piperazine-2,3-dione as the fourth eluting peak. ¹H NMR (500 MHz, methanol-d₄) δ=8.91 (s, 2H), 7.64-7.55 (m, 2H), 7.47-7.40 (m, 2H), 7.38-7.34 (m, 1H), 4.85 (s, 2H), 4.82-4.75 (m, 1H), 3.74-3.68 (m, 2H), 3.64-3.56 (m, 2H), 1.97-1.87 (m, 2H), 1.79-1.74 (m, 1H), 1.70-1.61 (m, 1H), 1.29-1.10 (m, 3H), 0.99 (d, J=6.1 Hz, 3H). LC/MS (m/z): 365 (M+H)⁺

### Example 345

### Preparation of 1-(tert-butyl)-4-((5-phenylpyrimidin-2-yl)methyl)piperazine-2,3-dione

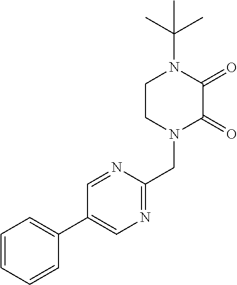 [SMILES: CC(C)(C)N1CCN(Cc2ncc(-c3ccccc3)cn2)C(=O)C1=O]

Sodium triacetoxyborohydride (194 mg, 0.916 mmol) was added to a mixture of 2-methylpropan-2-amine (25 mg, 0.34 mmol) and ethyl 2-oxo-2-((2-oxoethyl)((5-phenylpyrimidin-2-yl)methyl)amino)acetate (0.100 g, 0.31 mmol) in DCE (2 mL) at room temperature. The reaction mixture was stirred at room temperature for 12 hours and then stirred and heated at 65° C. for an additional 12 hours. The reaction mixture was cooled to room temperature and concentrated under reduced pressure. The residue was diluted with water (5 mL) and extracted with DCM (4×5 mL). The organic layers were combined, washed with brine (5 mL), dried over anhydrous sodium sulfate, filtered, and concentrated under reduced pressure. The residue was purified by reverse phase HPLC (eluting acetonitrile in water, with ammonium bicarbonate modifier) to afford 1-(tert-butyl)-4-((5-phenylpyrimidin-2-yl)methyl)piperazine-2,3-dione. ¹H NMR (400 MHz, methanol-d₄) δ 9.02 (s, 2H), 7.76-7.67 (m, 2H), 7.56-7.50 (m, 2H), 7.50-7.44 (m, 1H), 4.93 (s, 2H), 3.83-3.72 (m, 4H), 1.52 (s, 9H). LC/MS (m/z): 339 (M+H)⁺

### Example 346

### Preparation of 1-cyclopentyl-4-((5-phenyl-1,3,4-thiadiazol-2-yl)methyl)piperazine-2,3-dione

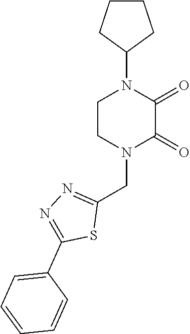 [SMILES: O=C1C(=O)N(C2CCCC2)CCN1Cc1nnc(-c2ccccc2)s1]

Acetic acid (26 μl, 0.45 mmol) and sodium triacetoxyborohydride (48 mg, 0.23 mmol) were added to a mixture of (5-phenyl-1,3,4-thiadiazol-2-yl)methanamine (29 mg, 0.15 mmol) and 4A powdered sieves (100 mg) in 1,2-dichloroethane (1 mL). A mixture of methyl 2-(cyclopentyl(2-oxoethyl)amino)-2-oxoacetate (42 mg, 0.20 mmol) in 1,2-dichloromethane (0.5 mL) was added, and the reaction mixture was stirred at room temperature for 18 hours. The reaction mixture was quenched with water and filtered. The filtrate was concentrated under reduced pressure. The residue was purified by reverse phase HPLC (eluting acetonitrile in water, with NH₄₀H as modifier) to afford 1-cyclopentyl-4-((5-phenyl-1,3,4-thiadiazol-2-yl)methyl)piperazine-2,3-dione. ¹H NMR (600 MHz, DMSO-d₆) δ 7.98 (d, J=7.7 Hz, 2H), 7.61-7.51 (m, 3H), 5.03 (s, 2H), 4.73-4.62 (m, 1H), 3.72-3.62 (m, 2H), 3.53-3.45 (m, 2H), 1.80-1.72 (m, 2H), 1.72-1.62 (m, 2H), 1.60-1.46 (m, 4H). LC/MS (m/z): 357 (M+H)⁺

Examples shown in Example Table 11 below, were or may be prepared according to procedures analogous to those outlined in Example 346 above using the appropriate starting materials, described in the Preparations or Intermediates above, or as obtained from commercial sources.

Example Table 11

| Example | Structure | Name | Mass [M + H]⁺ |
|---|---|---|---|
| Example 347 | 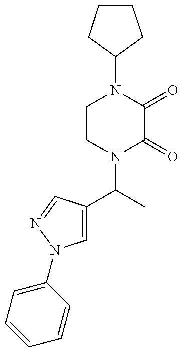 [SMILES: CC(c1cnn(-c2ccccc2)c1)N1CCN(C2CCCC2)C(=O)C1=O] | 1-cyclopentyl-4-(1-(1-phenyl-1H- pyrazol-4-yl)ethyl)piperazine-2,3- dione | 353 |
| Example 348 | 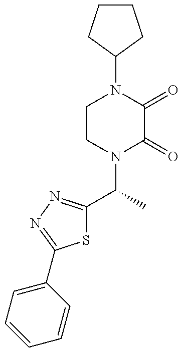 [SMILES: C[C@H](c1nnc(-c2ccccc2)s1)N1CCN(C2CCCC2)C(=O)C1=O] | (R)-1-cyclopentyl-4-(1-(5-phenyl- 1,3,4-thiadiazol-2- yl)ethyl)piperazine-2,3-dione | 371 |
| Example 349 | 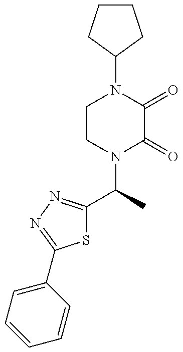 [SMILES: C[C@@H](c1nnc(-c2ccccc2)s1)N1CCN(C2CCCC2)C(=O)C1=O] | (S)-1-cyclopentyl-4-(1-(5-phenyl- 1,3,4-thiadiazol-2- yl)ethyl)piperazine-2,3-dione | 371 |
| Example 350 | 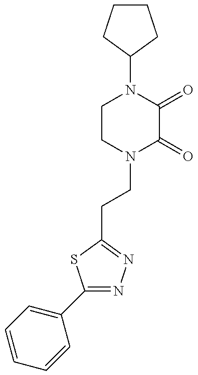 [SMILES: O=C1C(=O)N(C2CCCC2)CCN1CCc1nnc(-c2ccccc2)s1] | 1-cyclopentyl-4-(2-(5-phenyl-1,3,4- thiadiazol-2-yl)ethyl)piperazine- 2,3-dione | 371 |
| Example 351 | 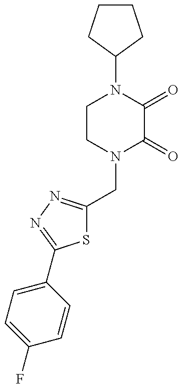 [SMILES: O=C1C(=O)N(C2CCCC2)CCN1Cc1nnc(-c2ccc(F)cc2)s1] | 1-cyclopentyl-4-((5-(4- fluorophenyl)-1,3,4-thiadiazol-2- yl)methyl)piperazine-2,3-dione | 375 |
| Example 352 | 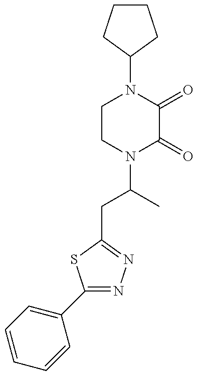 [SMILES: CC(Cc1nnc(-c2ccccc2)s1)N1CCN(C2CCCC2)C(=O)C1=O] | 1-cyclopentyl-4-(1-(5-phenyl-1,3,4- thiadiazol-2-yl)propan-2- yl)piperazine-2,3-dione | 385 |
| Example 353 | 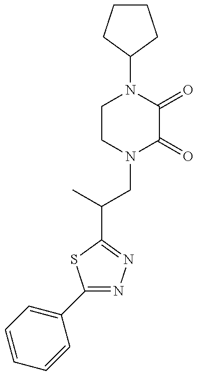 [SMILES: CC(CN1CCN(C2CCCC2)C(=O)C1=O)c1nnc(-c2ccccc2)s1] | 1-cyclopentyl-4-(2-(5-phenyl-1,3,4- thiadiazol-2-yl)propyl)piperazine- 2,3-dione | 385 |
| Example 354 | 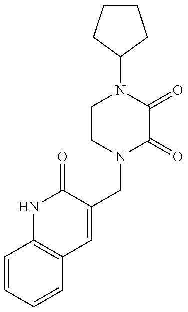 [SMILES: O=C1C(=O)N(C2CCCC2)CCN1Cc1cc2ccccc2[nH]c1=O] | 1-cyclopentyl-4-((2-oxo-1,2- dihydroquinolin-3- yl)methyl)piperazine-2,3-dione | 340 |
| Example 355 | 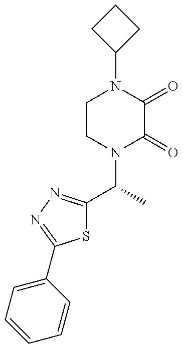 [SMILES: C[C@H](c1nnc(-c2ccccc2)s1)N1CCN(C2CCC2)C(=O)C1=O] | (R)-1-cyclobutyl-4-(1-(5-phenyl- 1,3,4-thiadiazol-2- yl)ethyl)piperazine-2,3-dione | 357 |
| Example 356 | 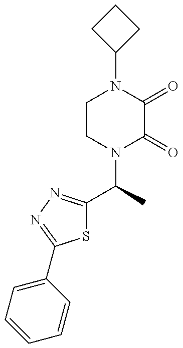 [SMILES: C[C@@H](c1nnc(-c2ccccc2)s1)N1CCN(C2CCC2)C(=O)C1=O] | (S)-1-cyclobutyl-4-(1-(5-phenyl- 1,3,4-thiadiazol-2- yl)ethyl)piperazine-2,3-dione | 357 |
| Example 357 | 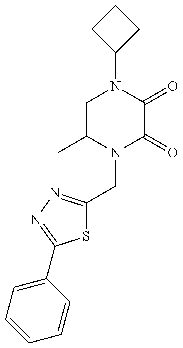 [SMILES: CC1CN(C2CCC2)C(=O)C(=O)N1Cc1nnc(-c2ccccc2)s1] | 1-cyclobutyl-5-methyl-4-((5- phenyl-1,3,4-thiadiazol-2- yl)methyl)piperazine-2,3-dione | 357 |

### Examples 358A and 358B

### Preparation of 1-cyclopentyl-4-((3-phenylisoxazol-5-yl)methyl)piperazine-2,3-dione and 1-cyclopentyl-4-((3-phenylisoxazol-5-yl)methyl)-1,4-dihydropyrazine-2,3-dione

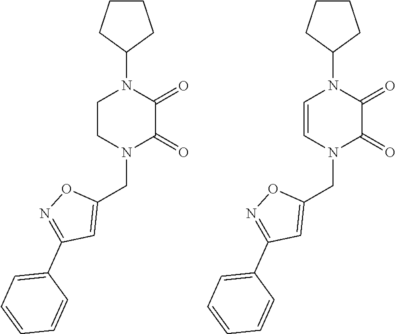 [SMILES: O=C1C(=O)N(C2CCCC2)CCN1Cc1cc(-c2ccccc2)no1 ; O=c1c(=O)n(C2CCCC2)ccn1Cc1cc(-c2ccccc2)no1]

A mixture of (3-phenylisoxazol-5-yl)methanamine (43 mg, 0.25 mmol), ethyl 2-(cyclopentyl(2-oxoethyl)amino)-2-oxoacetate (56 mg, 0.25 mmol), and sodium triacetoxyborohydride (131 mg, 0.616 mmol) in DCE (3 mL) was stirred at room temperature for 16 hours. The mixture was concentrated under reduced pressure, and the residue was partitioned between water (5 mL) and DCM (5 mL). The organic layer was separated, and the aqueous was re-extracted with DCM (3×5 mL). The organic layers were combined, washed with brine (5 mL), dried over anhydrous Na₂SO₄, filtered, and concentrated under reduced pressure. The residue was purified by reverse phase HPLC (eluting acetonitrile in water, with TFA modifier) to afford 1-cyclopentyl-4-((3-phenylisoxazol-5-yl)methyl)piperazine-2,3-dione and 1-cyclopentyl-4-((3-phenylisoxazol-5-yl)methyl)-1,4-dihydropyrazine-2,3-dione as an inseparable mixture. The mixture was further resolved by chiral-SFC (Column Daicel Chiralcel OD-H [250 mm×30 mm, 5 um]; eluting 55% [0.1% ammonia in ethanol] in CO₂) to afford 1-cyclopentyl-4-((3-phenylisoxazol-5-yl)methyl)piperazine-2,3-dione) as the first eluting peak and 1-cyclopentyl-4-((3-phenylisoxazol-5-yl)methyl)-1,4-dihydropyrazine-2,3-dione as the second eluting peak.

Peak 1: 1-cyclopentyl-4-((3-phenylisoxazol-5-yl)methyl)piperazine-2,3-dione): ¹H NMR (400 MHz, methanol-d₄) δ 7.86-7.81 (m, 2H), 7.51-7.45 (m, 3H), 6.89 (s, 1H), 4.89-4.88 (m, 2H), 4.81 (br t, J=8.3 Hz, 1H), 3.77-3.67 (m, 2H), 3.64-3.58 (m, 2H), 1.98-1.85 (m, 2H), 1.78 (br s, 2H), 1.69-1.59 (m, 4H). LC/MS (m/z): 340 (M+H)⁺

Peak 2: 1-cyclopentyl-4-((3-phenylisoxazol-5-yl)methyl)-1,4-dihydropyrazine-2,3-dione: ¹H NMR (400 MHz, methanol-d₄) δ 7.85-7.80 (m, 2H), 7.50-7.45 (m, 3H), 6.90 (s, 1H), 6.73 (d, J=6.4 Hz, 1H), 6.62 (d, J=6.4 Hz, 1H), 5.21 (s, 2H), 5.08-4.98 (m, 1H), 2.14-2.04 (m, 2H), 1.89 (br s, 2H), 1.80-1.70 (m, 4H). LC/MS (m/z): 338 (M+H)⁺

Examples shown in Example Table 12 below, were or may be prepared according to procedures analogous to those outlined in Examples 358A and 358B above using the appropriate starting materials, described in the Preparations or Intermediates above, or as obtained from commercial sources.

Example Table 12

| Example | Structure | Name | Mass [M + H]⁺ |
|---|---|---|---|
| Example 359 | 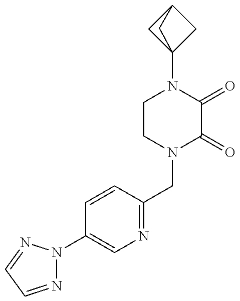 [SMILES: O=C1C(=O)N(C23CC(C2)C3)CCN1Cc1ccc(-n2nccn2)cn1] | 1-((5-(2H-1,2,3-triazol-2- yl)pyridin-2-yl)methyl)-4- (bicyclo[1.1.1]pentan-1- yl)piperazine-2,3-dione | 339 |
| Example 360 | 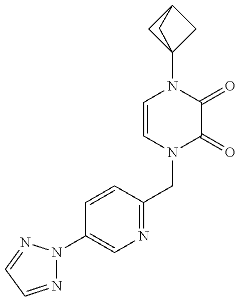 [SMILES: O=c1c(=O)n(C23CC(C2)C3)ccn1Cc1ccc(-n2nccn2)cn1] | 1-((5-(2H-1,2,3-triazol-2- yl)pyridin-2-yl)methyl)-4- (bicyclo[1.1.1]pentan-1- yl)-1,4-dihydropyrazine- 2,3-dione | 337 |

### Example 361

### Preparation of 1-cyclopentyl-4-((5-(thiazol-2-yl)pyrimidin-2-yl)methyl)piperazine-2,3-dione

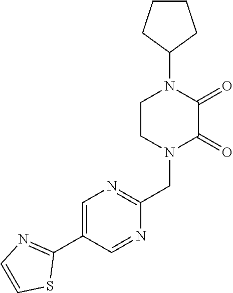 [SMILES: O=C1C(=O)N(C2CCCC2)CCN1Cc1ncc(-c2nccs2)cn1]

2-(Tributylstannyl)thiazole (0.052 ml, 0.11 mmol) was added to a mixture of 1-((5-bromopyrimidin-2-yl)methyl)-4-cyclopentylpiperazine-2,3-dione (0.030 g, 0.085 mmol) and Xphos-Pd G2 precatalyst (6.7 mg, 8.5 μmol) in 1,4-dioxane (0.5 mL) under a nitrogen atmosphere. The mixture was sparged with nitrogen for 1 minute. The reaction mixture was stirred and heated at 100° C. for 16 hours. The mixture was cooled to room temperature, diluted with DMSO, and filtered. The filtrate was purified by reverse phase HPLC (eluting acetonitrile in water, with TFA modifier) to afford 1-cyclopentyl-4-((5-(thiazol-2-yl)pyrimidin-2-yl)methyl)piperazine-2,3-dione. ¹H NMR (500 MHz, DMSO-d₆) δ 9.32 (s, 2H), 8.07 (d, J=3.2 Hz, 1H), 7.98 (d, J=3.2 Hz, 1H), 4.89 (s, 2H), 4.76-4.69 (m, 1H), 3.72-3.68 (m, 2H), 3.58-3.55 (m, 2H), 1.83-1.76 (m, 2H), 1.71-1.66 (m, 2H), 1.61-1.53 (m, 4H). LC/MS (m/z): 358 (M+H)⁺

Examples shown in Example Table 13 below, were or may be prepared according to procedures analogous to those outlined in Example 361 above using the appropriate starting materials, described in the Preparations or Intermediates above, or as obtained from commercial sources.

Example Table 13

| Example | Structure | Name | Mass [M + H]⁺ |
|---|---|---|---|
| Example 362 | 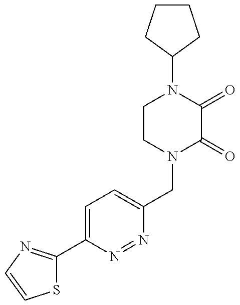 [SMILES: O=C1C(=O)N(C2CCCC2)CCN1Cc1ccc(-c2nccs2)nn1] | 1-cyclopentyl-4-((6- (thiazol-2-yl)pyridazin-3- yl)methyl)piperazine-2,3- dione | 358 |
| Example 363 | 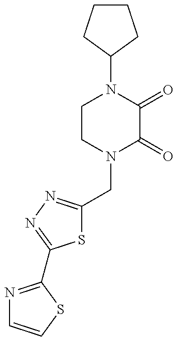 [SMILES: O=C1C(=O)N(C2CCCC2)CCN1Cc1nnc(-c2nccs2)s1] | 1-cyclopentyl-4-((5- (thiazol-2-yl)-1,3,4- thiadiazol-2- yl)methyl)piperazine-2,3- dione | 364 |
| Example 364 | 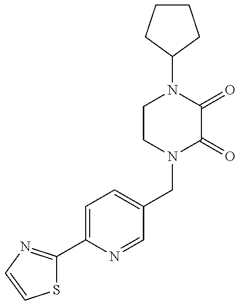 [SMILES: O=C1C(=O)N(C2CCCC2)CCN1Cc1ccc(-c2nccs2)nc1] | 1-cyclopentyl-4-((6- (thiazol-2-yl)pyridin-3- yl)methyl)piperazine-2,3- dione | 357 |
| Example 365 | 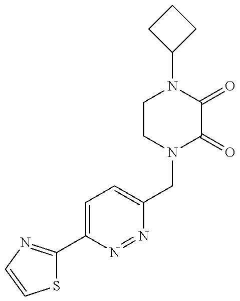 [SMILES: O=C1C(=O)N(C2CCC2)CCN1Cc1ccc(-c2nccs2)nn1] | 1-cyclobutyl-4-((6- (thiazol-2-yl)pyridazin-3- yl)methyl)piperazine-2,3- dione | 344 |
| Example 366 | 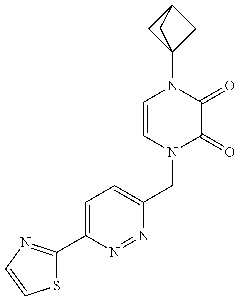 [SMILES: O=c1c(=O)n(C23CC(C2)C3)ccn1Cc1ccc(-c2nccs2)nn1] | 1-(bicyclo[1.1.1]pentan-1- yl)-4-((6-(thiazol-2- yl)pyridazin-3-yl)methyl)- 1,4-dihydropyrazine-2,3- dione | 354 |
| Example 367 | 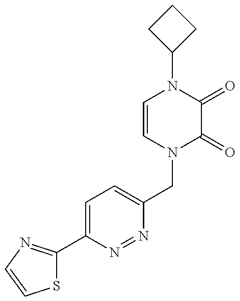 [SMILES: O=c1c(=O)n(C2CCC2)ccn1Cc1ccc(-c2nccs2)nn1] | 1-cyclobutyl-4-((6- (thiazol-2-yl)pyridazin-3- yl)methyl)-1,4- dihydropyrazine-2,3-dione | 342 |
| Example 368 | 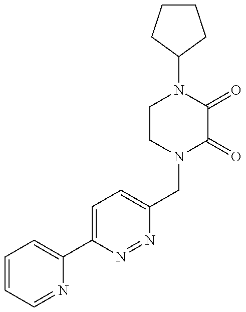 [SMILES: O=C1C(=O)N(C2CCCC2)CCN1Cc1ccc(-c2ccccn2)nn1] | 1-cyclopentyl-4-((6- (pyridin-2-yl)pyridazin-3- yl)methyl)piperazine-2,3- dione | 352 |
| Example 369 | 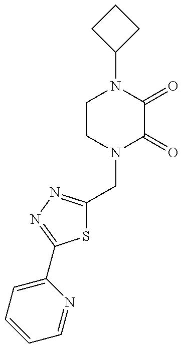 [SMILES: O=C1C(=O)N(C2CCC2)CCN1Cc1nnc(-c2ccccn2)s1] | 1-cyclobutyl-4-((5- (pyridin-2-yl)-1,3,4- thiadiazol-2- yl)methyl)piperazine-2,3- dione | 344 |
| Example 370 | 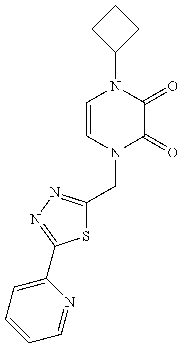 [SMILES: O=c1c(=O)n(C2CCC2)ccn1Cc1nnc(-c2ccccn2)s1] | 1-cyclobutyl-4-((5- (pyridin-2-yl)-1,3,4- thiadiazol-2-yl)methyl)- 1,4-dihydropyrazine-2,3- dione | 342 |
| Example 371 | 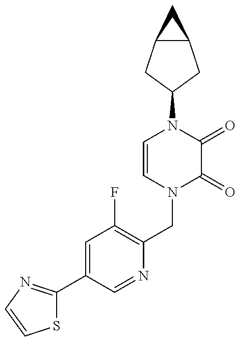 [SMILES: O=c1c(=O)n([C@H]2C[C@H]3C[C@H]3C2)ccn1Cc1ncc(-c2nccs2)cc1F] | 1-((cis)- bicyclo[3.1.0]hexan-3-yl)- 4-((3-fluoro-5-(thiazol-2- yl)pyridin-2-yl)methyl)- 1,4-dihydropyrazine-2,3- dione | 385 |
| Example 372 | 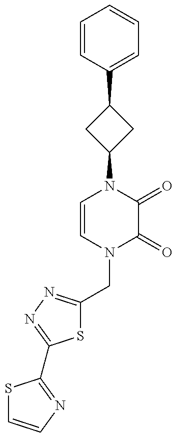 [SMILES: O=c1c(=O)n([C@H]2C[C@@H](c3ccccc3)C2)ccn1Cc1nnc(-c2nccs2)s1] | 1-((cis)-3- phenylcyclobutyl)-4-((5- (thiazol-2-yl)-1,3,4- thiadiazol-2-yl)methyl)- 1,4-dihydropyrazine-2,3- dione | 424 |
| Example 373 | 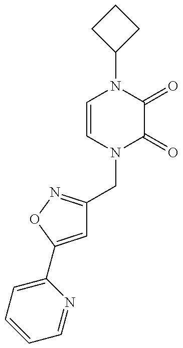 [SMILES: O=c1c(=O)n(C2CCC2)ccn1Cc1cc(-c2ccccn2)on1] | 1-cyclobutyl-4-((5- (pyridin-2-yl)isoxazol-3- yl)methyl)-1,4- dihydropyrazine-2,3-dione | 325 |
| Example 374 | 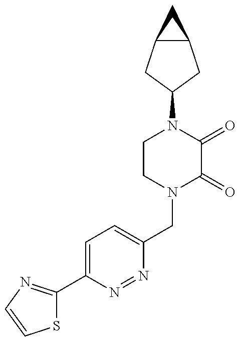 [SMILES: O=C1C(=O)N([C@H]2C[C@H]3C[C@H]3C2)CCN1Cc1ccc(-c2nccs2)nn1] | 1-((cis)- bicyclo[3.1.0]hexan-3-yl)- 4-((6-(thiazol-2- yl)pyridazin-3- yl)methyl)piperazine-2,3- dione | 370 |
| Example 375 | 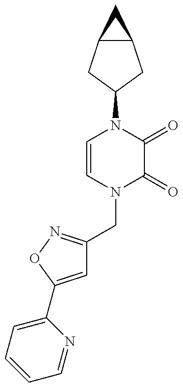 [SMILES: O=c1c(=O)n([C@H]2C[C@H]3C[C@H]3C2)ccn1Cc1cc(-c2ccccn2)on1] | 1-((cis)- bicyclo[3.1.0]hexan-3-yl)- 4-((5-(pyridin-2- yl)isoxazol-3-yl)methyl)- 1,4-dihydropyrazine-2,3- dione | 351 |
| Example 376 | 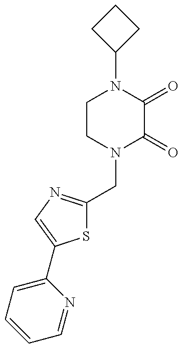 [SMILES: O=C1C(=O)N(C2CCC2)CCN1Cc1ncc(-c2ccccn2)s1] | 1-cyclobutyl-4-((5- (pyridin-2-yl)thiazol-2- yl)methyl)piperazine-2,3- dione | 343 |
| Example 377 | 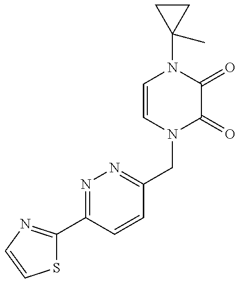 [SMILES: CC1(n2ccn(Cc3ccc(-c4nccs4)nn3)c(=O)c2=O)CC1] | 1-(1-methylcyclopropyl)- 4-((6-(thiazol-2- yl)pyridazin-3-yl)methyl)- 1,4-dihydropyrazine-2,3- dione | 342 |

### Example 378

### Preparation of 1-((5-(2H-1,2,3-triazol-2-yl)pyrimidin-2-yl)methyl)-4-cyclopentylpiperazine-2,3-dione

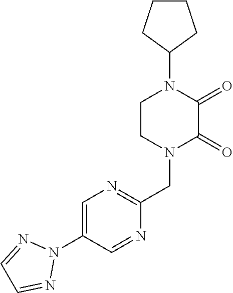 [SMILES: O=C1C(=O)N(C2CCCC2)CCN1Cc1ncc(-n2nccn2)cn1]

A mixture of 1-((5-bromopyrimidin-2-yl)methyl)-4-cyclopentylpiperazine-2,3-dione (0.050 g, 0.14 mmol), cesium carbonate (138 mg, 0.425 mmol), bis((Z)-2-methyl-1-(2-oxocyclohexylidene)propoxy)copper (28 mg, 0.071 mmol), and 2H-1,2,3-triazole (0.014 ml, 0.24 mmol) in 1,4-dioxane (1.5 mL) was sparged with nitrogen for 1 minute. The reaction mixture was stirred and heated at 120° C. for 16 hours. The mixture was cooled to room temperature, diluted with DCM (3 mL), and filtered. The filtrate was purified by silica gel chromatography (eluting methanol in DCM) and by basic alumina chromatography (eluting [1:3 EtOH:EtOAc] in hexanes) to afford 1-((5-(2H-1,2,3-triazol-2-yl)pyrimidin-2-yl)methyl)-4-cyclopentylpiperazine-2,3-dione. ¹H NMR (500 MHz, DMSO-d₆) δ 9.41 (s, 2H), 8.29 (s, 2H), 4.91 (s, 2H), 4.76-4.69 (m, 1H), 3.72-3.68 (m, 2H), 3.57-3.54 (m, 2H), 1.84-1.76 (m, 2H), 1.72-1.66 (m, 2H), 1.61-1.52 (m, 4H). LC/MS (m/z): 342 (M+H)⁺

Examples shown in Example Table 14 below, were or may be prepared according to procedures analogous to those outlined in Example 378 above using the appropriate starting materials, described in the Preparations or Intermediates above, or as obtained from commercial sources.

Example Table 14

| Example | Structure | Name | Mass [M + H]⁺ |
|---|---|---|---|
| Example 379 | 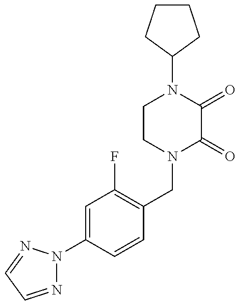 [SMILES: O=C1C(=O)N(C2CCCC2)CCN1Cc1ccc(-n2nccn2)cc1F] | 1-cyclopentyl-4-(2-fluoro-4- (2H-1,2,3-triazol-2- yl)benzyl)piperazine-2,3-dione | 358 |
| Example 380 | 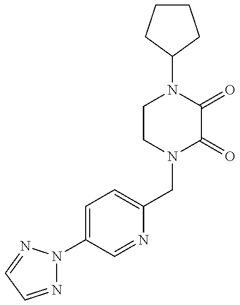 [SMILES: O=C1C(=O)N(C2CCCC2)CCN1Cc1ccc(-n2nccn2)cn1] | 1-((5-(2H-1,2,3-triazol-2- yl)pyridin-2-yl)methyl)-4- cyclopentylpiperazine-2,3-dione | 341 |
| Example 381 | 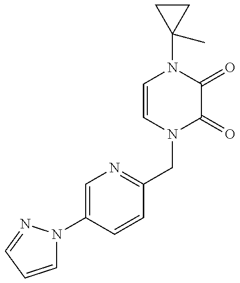 [SMILES: CC1(n2ccn(Cc3ccc(-n4cccn4)cn3)c(=O)c2=O)CC1] | 1-((5-(1H-pyrazol-1-yl)pyridin- 2-yl)methyl)-4-(1- methylcyclopropyl)-1,4- dihydropyrazine-2,3-dione | 324 |
| Example 382 | 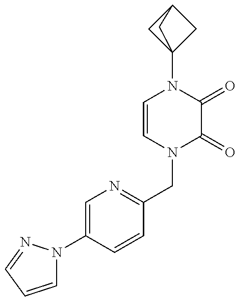 [SMILES: O=c1c(=O)n(C23CC(C2)C3)ccn1Cc1ccc(-n2cccn2)cn1] | 1-((5-(1H-pyrazol-1-yl)pyridin- 2-yl)methyl)-4- (bicyclo[1.1.1]pentan-1-yl)-1,4- dihydropyrazine-2,3-dione | 336 |
| Example 383 | 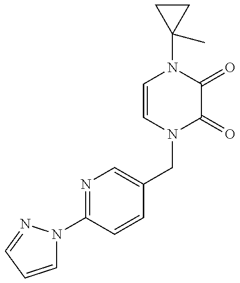 [SMILES: CC1(n2ccn(Cc3ccc(-n4cccn4)nc3)c(=O)c2=O)CC1] | 1-((6-(1H-pyrazol-1-yl)pyridin- 3-yl)methyl)-4-(1- methylcyclopropyl)-1,4- dihydropyrazine-2,3-dione | 324 |
| Example 384 | 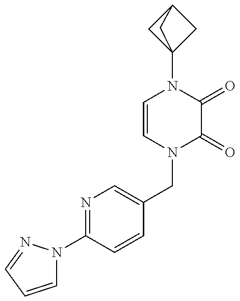 [SMILES: O=c1c(=O)n(C23CC(C2)C3)ccn1Cc1ccc(-n2cccn2)nc1] | 1-((6-(1H-pyrazol-1-yl)pyridin- 3-yl)methyl)-4- (bicyclo[1.1.1]pentan-1-yl)-1,4- dihydropyrazine-2,3-dione | 336 |
| Example 385 | 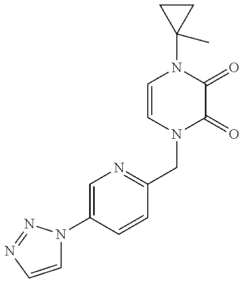 [SMILES: CC1(n2ccn(Cc3ccc(-n4ccnn4)cn3)c(=O)c2=O)CC1] | 1-((5-(1H-1,2,3-triazol-1- yl)pyridin-2-yl)methyl)-4-(1- methylcyclopropyl)-1,4- dihydropyrazine-2,3-dione | 325 |
| Example 386 | 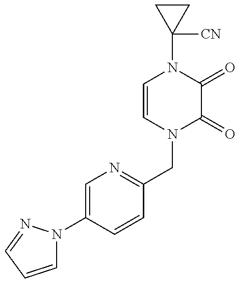 [SMILES: N#CC1(n2ccn(Cc3ccc(-n4cccn4)cn3)c(=O)c2=O)CC1] | 1-(4-((5-(1H-pyrazol-1- yl)pyridin-2-yl)methyl)-2,3- dioxo-3,4-dihydropyrazin- 1(2H)-yl)cyclopropane-1- carbonitrile | 335 |
| Example 387 | 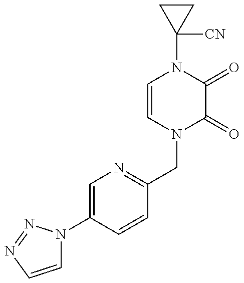 [SMILES: N#CC1(n2ccn(Cc3ccc(-n4ccnn4)cn3)c(=O)c2=O)CC1] | 1-(4-((5-(1H-1,2,3-triazol-1- yl)pyridin-2-yl)methyl)-2,3- dioxo-3,4-dihydropyrazin- 1(2H)-yl)cyclopropane-1- carbonitrile | 336 |
| Example 388 | 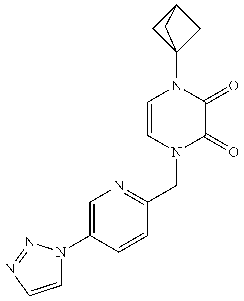 [SMILES: O=c1c(=O)n(C23CC(C2)C3)ccn1Cc1ccc(-n2ccnn2)cn1] | 1-((5-(1H-1,2,3-triazol-1- yl)pyridin-2-yl)methyl)-4- (bicyclo[1.1.1]pentan-1-yl)-1,4- dihydropyrazine-2,3-dione | 337 |
| Example 389 | 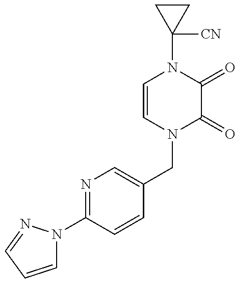 [SMILES: N#CC1(n2ccn(Cc3ccc(-n4cccn4)nc3)c(=O)c2=O)CC1] | 1-(4-((6-(1H-pyrazol-1- yl)pyridin-3-yl)methyl)-2,3- dioxo-3,4-dihydropyrazin- 1(2H)-yl)cyclopropane-1- carbonitrile | 335 |
| Example 390 | 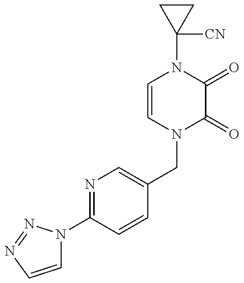 [SMILES: N#CC1(n2ccn(Cc3ccc(-n4ccnn4)nc3)c(=O)c2=O)CC1] | 1-(4-((6-(1H-1,2,3-triazol-1- yl)pyridin-3-yl)methyl)-2,3- dioxo-3,4-dihydropyrazin- 1(2H)-yl)cyclopropane-1- carbonitrile | 336 |
| Example 391 | 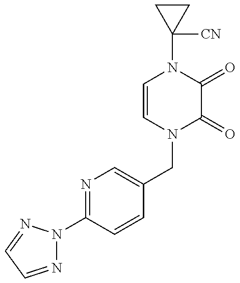 [SMILES: N#CC1(n2ccn(Cc3ccc(-n4nccn4)nc3)c(=O)c2=O)CC1] | 1-(4-((6-(2H-1,2,3-triazol-2- yl)pyridin-3-yl)methyl)-2,3- dioxo-3,4-dihydropyrazin- 1(2H)-yl)cyclopropane-1- carbonitrile | 336 |

### Example 392

### Preparation of 1-cyclopentyl-4-((6-(3,3-difluoropyrrolidin-1-yl)pyridin-3-yl)methyl)piperazine-2,3-dione

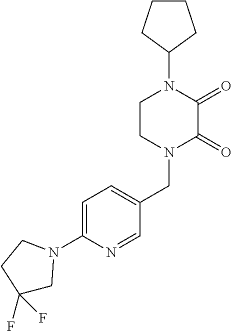 [SMILES: O=C1C(=O)N(C2CCCC2)CCN1Cc1ccc(N2CCC(F)(F)C2)nc1]

A mixture of 1-((6-chloropyridin-3-yl)methyl)-4-cyclopentylpiperazine-2,3-dione (0.031 g, 0.10 mmol), 3,3-difluoropyrrolidine hydrochloride (0.021 g, 0.15 mmol), RuPhos Pd G3 precatalyst (0.012 g, 0.015 mmol), and sodium tert-butoxide (0.029 g, 0.30 mmol) in 1,4-dioxane (1 mL) was purged with argon for 5 minutes. The reaction mixture was stirred and heated to 80° C. for 1 hour. The reaction mixture was cooled to room temperature, quenched with saturated aqueous NH₄Cl, and extracted with DCM. The organic layer was concentrated under reduced pressure. The residue was purified by reverse phase HPLC (eluting acetonitrile in water, with TFA modifier) to afford 1-cyclopentyl-4-((6-(3,3-difluoropyrrolidin-1-yl)pyridin-3-yl)methyl)piperazine-2,3-dione. ¹H NMR (500 MHz, DMSO-d₆) δ 8.07-7.99 (s, 1H), 7.58 (d, J=8.7 Hz, 1H), 6.64 (d, J=8.7 Hz, 1H), 4.65 (q, J=8.2 Hz, 1H), 4.45 (s, 2H), 3.84 (t, J=13.2 Hz, 2H), 3.54-3.47 (m, 4H), 3.44-3.34 (m, 4H), 1.80-1.69 (m, 2H), 1.63 (m, 2H), 1.50 (m, 4H). LC/MS (m/z): 379 (M+H)⁺

Examples shown in Example Table 15 below, were or may be prepared according to procedures analogous to those outlined in Example 392 above using the appropriate starting materials, described in the Preparations or Intermediates above, or as obtained from commercial sources.

Example Table 15

| Example | Structure | Name | Mass [M + H]⁺ |
|---|---|---|---|
| Example 393 | 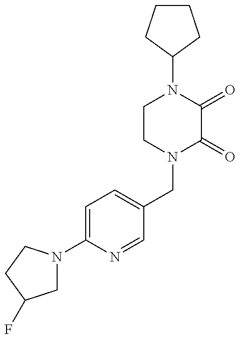 [SMILES: O=C1C(=O)N(C2CCCC2)CCN1Cc1ccc(N2CCC(F)C2)nc1] | 1-cyclopentyl-4-((6-(3- fluoropyrrolidin-1-yl)pyridin- 3-yl)methyl)piperazine-2,3- dione | 361 |

### Example 394

### Preparation of 1-cyclobutyl-4-((6-(oxazol-5-yl)pyridazin-3-yl)methyl)piperazine-2,3-dione

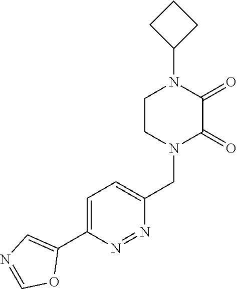 [SMILES: O=C1C(=O)N(C2CCC2)CCN1Cc1ccc(-c2cnco2)nn1]

A mixture of 1-((6-chloropyridazin-3-yl)methyl)-4-cyclobutylpiperazine-2,3-dione (0.070 g, 0.24 mmol), K₂CO₃ (82 mg, 0.59 mmol), oxazole (0.031 mL, 0.48 mmol), pivalic acid (10 mg, 0.1 mmol), palladium(II) acetate (3 mg, 0.01 mmol), and butyl di-1-adamantylphosphine (10 mg, 0.03 mmol) in DMA (3 mL) was sparged with N₂ at room temperature. The reaction mixture was stirred and heated to 110° C. for 12 hours. The mixture was cooled to room temperature and filtered. The filtrate was purified by reverse phase HPLC (eluting acetonitrile in water, with TFA modifier) to afford 1-cyclobutyl-4-((6-(oxazol-5-yl)pyridazin-3-yl)methyl)piperazine-2,3-dione. ¹H NMR (500 MHz, methanol-d₄) δ 8.48 (s, 1H), 8.10 (d, J=8.8 Hz, 1H), 7.99 (s, 1H), 7.85 (d, J=8.9 Hz, 1H), 5.01 (s, 2H), 4.88-4.84 (m, 1H), 3.84-3.79 (m, 2H), 3.76-3.71 (m, 2H), 2.32-2.25 (m, 2H), 2.25-2.17 (m, 2H), 1.89-1.69 (m, 2H). LC/MS (m/z): 328 (M+H)⁺

Examples shown in Example Table 16 below, were or may be prepared according to procedures analogous to those outlined in Example 394 above using the appropriate starting materials, described in the Preparations or Intermediates above, or as obtained from commercial sources.

Example Table 16

| Example | Structure | Name | Mass [M + H]⁺ |
|---|---|---|---|
| Example 395 | 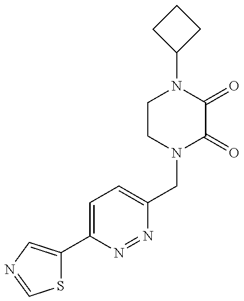 [SMILES: O=C1C(=O)N(C2CCC2)CCN1Cc1ccc(-c2cncs2)nn1] | 1-cyclobutyl-4-((6-(thiazol-5- yl)pyridazin-3- yl)methyl)piperazine-2,3-dione | 344 |

### Example 396

### Preparation of 1-cyclobutyl-4-((5-phenyl-1,3,4-thiadiazol-2-yl)methyl)-1,4-dihydropyrazine-2,3-dione

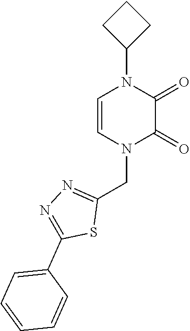 [SMILES: O=c1c(=O)n(C2CCC2)ccn1Cc1nnc(-c2ccccc2)s1]

### Step A: N-(2-chloroacetyl)benzohydrazide

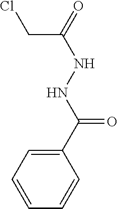 [SMILES: O=C(CCl)NNC(=O)c1ccccc1]

Chloroacetyl chloride (6.7 mL, 84 mmol) was added dropwise to a mixture of benzohydrazide (9.5 g, 70 mmol) in ethyl acetate (140 mL) at 0° C. The reaction mixture was stirred and heated at 80° C. for 3 hours. The mixture was concentrated under reduced pressure to give afford N′-(2-chloroacetyl)benzohydrazide which was used without purification. LC/MS (m/z): 213 (M+H)⁺

### Step B: 2-(chloromethyl)-5-phenyl-1,3,4-thiadiazole

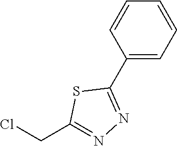 [SMILES: ClCc1nnc(-c2ccccc2)s1]

Lawesson's reagent (17 g, 42 mmol) was added to a mixture of N-(2-chloroacetyl)benzohydrazide (15 g, 70 mmol) in THE (300 mL). The mixture was stirred and heated at 70° C. for 4 hours. The reaction mixture was quenched with NaHCO₃ (1 N, 300 mL) and extracted with EtOAc (3×300 mL). The organic layers were combined, washed with brine (500 mL), dried over anhydrous sodium sulfate, filtered, and concentrated under reduced pressure. The residue was purified by silica gel chromatography (eluting ethyl acetate in petroleum ether) to afford 2-(chloromethyl)-5-phenyl-1,3,4-thiadiazole. ¹H NMR (500 MHz, chloroform-d) δ 7.96 (dd, J=1.5, 7.9 Hz, 2H), 7.56-7.47 (m, 3H), 4.98 (s, 2H). LC/MS (m/z): 211 (M+H)⁺

### Step C: N-((5-phenyl-1,3,4-thiadiazol-2-yl)methyl)prop-2-en-1-amine

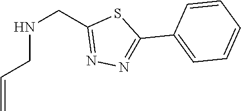 [SMILES: C=CCNCc1nnc(-c2ccccc2)s1]

Sodium iodide (1.28 g, 8.54 mmol) and 2-(chloromethyl)-5-phenyl-1,3,4-thiadiazole (1.8 g, 8.5 mmol) were added to a stirred mixture of K₂CO₃ (1.54 g, 11.1 mmol) and prop-2-en-1-amine (1.75 mL, 23.3 mmol) in THE (30 mL) at 0° C. under a nitrogen atmosphere. The reaction mixture was stirred at room temperature for 12 hours. The mixture was filtered, and the filtrate was concentrated under reduced pressure to afford N-((5-phenyl-1,3,4-thiadiazol-2-yl)methyl)prop-2-en-1-amine, which was used without purification. LC/MS (m/z): 232 (M+H)⁺

### Step D: ethyl 2-(allyl((5-phenyl-1,3,4-thiadiazol-2-yl)methyl)amino)-2-oxoacetate

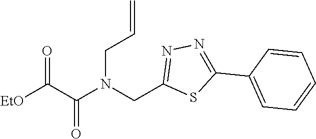 [SMILES: C=CCN(Cc1nnc(-c2ccccc2)s1)C(=O)C(=O)OCC]

Triethylamine (3.6 mL, 26 mmol) was added to a mixture of N-((5-phenyl-1,3,4-thiadiazol-2-yl)methyl)prop-2-en-1-amine (2.0 g, 8.5 mmol) in DCM (40 mL). The mixture was stirred for 5 min at 0° C., and then ethyl 2-chloro-2-oxoacetate (1.37 mL, 12.8 mmol) was added to the mixture at 0° C. The reaction mixture was stirred at room temperature for an additional 2 hours. The mixture was concentrated under reduced pressure, and the residue was partitioned between water (80 mL) and EtOAc (60 mL). The organic layer was separated, and the aqueous layer was re-extracted with EtOAc (3×60 mL). The organic layers were combined, washed with brine (100 mL), dried over anhydrous Na₂SO₄, filtered, and concentrated under reduced pressure. The residue was purified by silica gel chromatography (eluting ethyl acetate in petroleum ether) to afford ethyl 2-(allyl((5-phenyl-1,3,4-thiadiazol-2-yl)methyl)amino)-2-oxoacetate. ¹H NMR (400 MHz, chloroform-d) δ 8.01-7.92 (m, 2H), 7.54-7.45 (m, 3H), 5.92-5.73 (m, 1H), 5.42-5.27 (m, 2H), 4.99-4.91 (m, 2H), 4.46-4.32 (m, 2H), 4.12-4.01 (m, 2H), 1.44-1.35 (m, 3H). LC/MS (m/z): 332 (M+H)⁺

### Step E: ethyl 2-oxo-2-((2-oxoethyl)((5-phenyl-1,3,4-thiadiazol-2-yl)methyl)amino)acetate

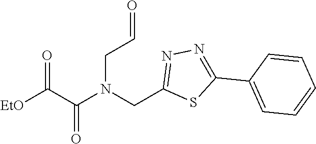 [SMILES: CCOC(=O)C(=O)N(CC=O)Cc1nnc(-c2ccccc2)s1]

(Osmium tetroxide) (15 mg, 60 μmol) was added to a mixture of ethyl 2-(allyl((5-phenyl-1,3,4-thiadiazol-2-yl)methyl)amino)-2-oxoacetate (100 mg, 300 μmol) and NaIO₅ (194 mg, 905 μmol) in THE (1.5 mL) and water (0.5 mL) at 0° C. The reaction mixture was stirred at room temperature for 1 hour. The reaction mixture was quenched with sodium bisulfite (saturated aqueous solution) and then stirred for 15 minutes. The mixture was diluted with water (10 mL) and extracted with EtOAc (3×10 mL). The organic layers were combined, washed with brine (20 mL), dried over anhydrous Na₂SO₄, filtered, and concentrated under reduced pressure to afford ethyl 2-oxo-2-((2-oxoethyl)((5-phenyl-1,3,4-thiadiazol-2-yl)methyl)amino)acetate, which was used without purification. LC/MS (m/z): 334 (M+H)⁺

### Step F: 1-cyclobutyl-4-((5-phenyl-1,3,4-thiadiazol-2-yl)methyl)-1,4-dihydropyrazine-2,3-dione

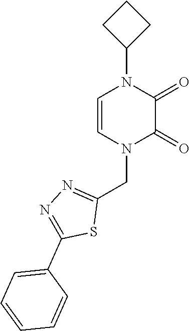 [SMILES: O=c1c(=O)n(C2CCC2)ccn1Cc1nnc(-c2ccccc2)s1]

Sodium triacetoxyborohydride (127 mg, 600 μmol) was added to a mixture of ethyl 2-oxo-2-((2-oxoethyl)((5-phenyl-1,3,4-thiadiazol-2-yl)methyl)amino)acetate (100 mg, 300 μmol) and cyclobutanamine (24 mg, 330 μmol) in 1,2-dichloroethane (3 mL) at room temperature. The reaction mixture was stirred at room temperature for 3 hours. The reaction mixture was quenched with MeOH (5 mL) and then concentrated under reduced pressure. The residue was purified by reverse phase HPLC (eluting acetonitrile in water, with ammonia modifier) to give a mixture of 1-cyclobutyl-4-((5-phenyl-1,3,4-thiadiazol-2-yl)methyl)piperazine-2,3-dione and 1-cyclobutyl-4-((5-phenyl-1,3,4-thiadiazol-2-yl)methyl)-1,4-dihydropyrazine-2,3-dione. The mixture was further resolved by Chiral-SFC (Chiral OD-H (250 mm*30 mm) 50% EtOH with 0.1% ammonia modifier) to afford 1-cyclobutyl-4-((5-phenyl-1,3,4-thiadiazol-2-yl)methyl)-1,4-dihydropyrazine-2,3-dione as the second eluting peak. ¹H NMR (400 MHz, methanol-d₄) δ 7.96 (dd, J=1.6, 7.8 Hz, 2H), 7.59-7.49 (m, 3H), 6.79 (s, 2H), 5.45 (s, 2H), 4.99-4.93 (m, 1H), 2.42-2.26 (m, 4H), 1.90-1.79 (m, 2H). LC/MS (m/z): 341 (M+H)⁺

### Example 397

### Preparation of 1-((5-(2H-1,2,3-triazol-2-yl)pyridin-2-yl)methyl)-4-cyclobutyl-1,4-dihydropyrazine-2,3-dione

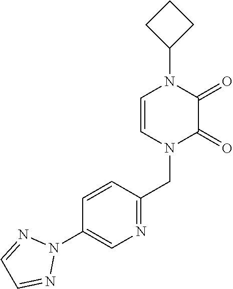 [SMILES: O=c1c(=O)n(C2CCC2)ccn1Cc1ccc(-n2nccn2)cn1]

A mixture of 1-((5-bromopyridin-2-yl)methyl)-4-cyclobutyl-1,4-dihydropyrazine-2,3-dione (2.50 g, 7.44 mmol), 2H-1,2,3-triazole (0.56 mL, 9.7 mmol), Pd₂(dba)₃ (0.68 g, 0.74 mmol), 2-di-tert-butylphosphino-3,4,5,6-tetramethyl-2′,4′,6′-triisopropyl-1,1′-biphenyl (0.89 g, 1.9 mmol) and potassium phosphate tribasic (3.16 g, 14.9 mmol) was degassed with argon for 5 minutes. Toluene (74 mL) was added, and the mixture was degassed with argon for an additional 5 minutes. The mixture was heated to 100° C. and stirred under an argon atmosphere for 18 hours. Additional Pd₂(dba)₃ (0.34 g, 0.37 mmol) and 2-di-tert-butylphosphino-3,4,5,6-tetramethyl-2′,4′,6′-triisopropyl-1,1′-biphenyl (0.43 g, 0.93 mmol) were added to the mixture, and the mixture was stirred and heated at 110° C. for an additional 24 hours. The reaction mixture was cooled to room temperature and diluted with dichloromethane (100 mL). The mixture was filtered, and the filtrate was concentrated under reduced pressure. The residue was purified by silica gel chromatography (eluting with [3:1 ethyl acetate:ethanol] in dichloromethane) to afford the product as a mixture with impurities. The residue was suspended in ethyl acetate (200 mL) and stirred for 18 hours. The mixture was filtered, and the collected solids were re-purified by silica gel chromatography (eluting with [3:1 ethyl acetate:ethanol] in dichloromethane) followed by purification by SFC (eluting ammonia in methanol in CO₂) to afford 1-((5-(2H-1,2,3-triazol-2-yl)pyridin-2-yl)methyl)-4-cyclobutyl-1,4-dihydropyrazine-2,3-dione. ¹H NMR (499 MHz, DMSO-d₆) δ 9.18 (d, J=2.4 Hz, 1H), 8.38 (dd, J=8.5, 2.6 Hz, 1H), 8.21 (s, 2H), 7.55 (d, J=8.5 Hz, 1H), 6.82 (d, J=6.2 Hz, 1H), 6.75 (d, J=6.2 Hz, 1H), 5.11 (s, 2H), 4.92 (p, J=9.2, 8.7 Hz, 1H), 2.35-2.19 (m, 4H), 1.85-1.63 (m, 2H). LC/MS (m/z): 325 (M+H)⁺

### Example 398

### Preparation of 1-((6-(2H-1,2,3-triazol-2-yl)pyridazin-3-yl)methyl)-4-cyclobutylpiperazine-2,3-dione

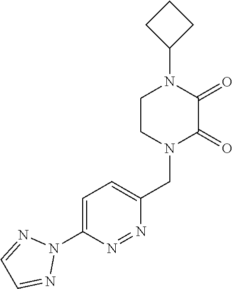 [SMILES: O=C1C(=O)N(C2CCC2)CCN1Cc1ccc(-n2nccn2)nn1]

A mixture of Pd₂(dba)₃ (8 mg, 8 μmol) and tri-tert-butylphosphonium tetrafluoroborate (5 mg, 0.02 mmol) were premixed in toluene (2 mL) and stirred at room temperature for 3 minutes under a nitrogen atmosphere. 1-((6-chloropyridazin-3-yl)methyl)-4-cyclobutylpiperazine-2,3-dione (0.050 g, 0.17 mmol), 2H-1,2,3-triazole (23 mg, 0.34 mmol) and K₃PO₄ (108 mg, 0.509 mmol) were added to the reaction mixture at room temperature under a nitrogen atmosphere. The reaction mixture was stirred and heated to 110° C. for 12 hours. The reaction mixture was cooled to room temperature and concentrated under reduced pressure. The residue was purified by reverse phase HPLC (eluting acetonitrile in water, with TFA modifier) to afford the product as mixture of triazole isomers, which was further resolved by Chiral-SFC (Chiralcel OJ-H (250 mm×30 mm, Sum); eluting 50% ethanol with 0.1% ammonia modifier) to afford 1-((6-(2H-1,2,3-triazol-2-yl)pyridazin-3-yl)methyl)-4-cyclobutylpiperazine-2,3-dione as the second eluting peak. ¹H NMR (500 MHz, methanol-d₄) δ 8.31 (d, J=9.2 Hz, 1H), 8.04 (s, 2H), 7.86 (d, J=9.0 Hz, 1H), 4.92 (s, 2H), 4.77-4.70 (m, 1H), 3.74-3.59 (m, 4H), 2.24-2.04 (m, 4H), 1.73-1.60 (m, 2H). LC/MS (m/z): 328 (M+H)⁺

Examples shown in Example Table 17 below, were or may be prepared according to procedures analogous to those outlined in Example 398 above using the appropriate starting materials, described in the Preparations or Intermediates above, or as obtained from commercial

Example Table 17

| Example | Structure | Name | Mass [M + H]⁺ |
|---|---|---|---|
| Example 399 | 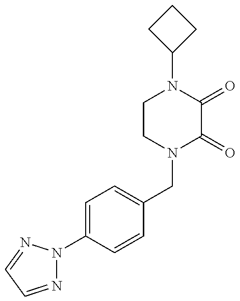 [SMILES: O=C1C(=O)N(C2CCC2)CCN1Cc1ccc(-n2nccn2)cc1] | 1-(4-(2H-1,2,3-triazol-2- yl)benzyl)-4- cyclobutylpiperazine-2,3-dione | 326 |
| Example 400 | 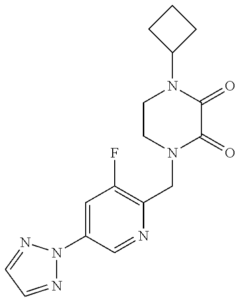 [SMILES: O=C1C(=O)N(C2CCC2)CCN1Cc1ncc(-n2nccn2)cc1F] | 1-cyclobutyl-4-((3-fluoro-5-(2H- 1,2,3-triazol-2-yl)pyridin-2- yl)methyl)piperazine-2,3-dione | 345 |
| Example 401 | 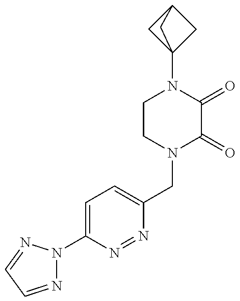 [SMILES: O=C1C(=O)N(C23CC(C2)C3)CCN1Cc1ccc(-n2nccn2)nn1] | 1-((6-(2H-1,2,3-triazol-2- yl)pyridazin-3-yl)methyl)-4- (bicyclo[1.1.1]pentan-1- yl)piperazine-2,3-dione | 340 |
| Example 402 | 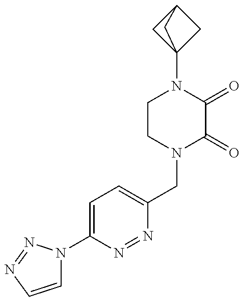 [SMILES: O=C1C(=O)N(C23CC(C2)C3)CCN1Cc1ccc(-n2ccnn2)nn1] | 1-((6-(1H-1,2,3-triazol-1- yl)pyridazin-3-yl)methyl)-4- (bicyclo[1.1.1]pentan-1- yl)piperazine-2,3-dione | 340 |
| Example 403 | 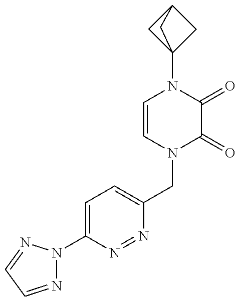 [SMILES: O=c1c(=O)n(C23CC(C2)C3)ccn1Cc1ccc(-n2nccn2)nn1] | 1-((6-(2H-1,2,3-triazol-2- yl)pyridazin-3-yl)methyl)-4- (bicyclo[1.1.1]pentan-1-yl)-1,4- dihydropyrazine-2,3-dione | 338 |
| Example 404 | 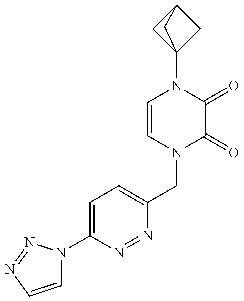 [SMILES: O=c1c(=O)n(C23CC(C2)C3)ccn1Cc1ccc(-n2ccnn2)nn1] | 1-((6-(1H-1,2,3-triazol-1- yl)pyridazin-3-yl)methyl)-4- (bicyclo[1.1.1]pentan-1-yl)-1,4- dihydropyrazine-2,3-dione | 338 |
| Example 405 | 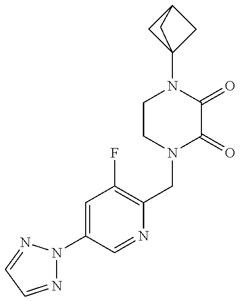 [SMILES: O=C1C(=O)N(C23CC(C2)C3)CCN1Cc1ncc(-n2nccn2)cc1F] | 1-(bicyclo[1.1.1]pentan-1-yl)-4- ((3-fluoro-5-(2H-1,2,3-triazol-2- yl)pyridin-2- yl)methyl)piperazine-2,3-dione | 357 |
| Example 406 | 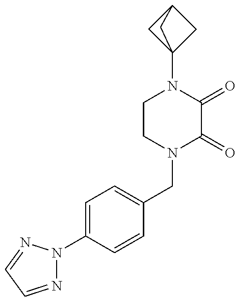 [SMILES: O=C1C(=O)N(C23CC(C2)C3)CCN1Cc1ccc(-n2nccn2)cc1] | 1-(4-(2H-1,2,3-triazol-2- yl)benzyl)-4- (bicyclo[1.1.1]pentan-1- yl)piperazine-2,3-dione | 338 |
| Example 407 | 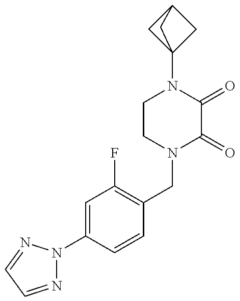 [SMILES: O=C1C(=O)N(C23CC(C2)C3)CCN1Cc1ccc(-n2nccn2)cc1F] | 1-(bicyclo[1.1.1]pentan-1-yl)-4- (2-fluoro-4-(2H-1,2,3-triazol-2- yl)benzyl)piperazine-2,3-dione | 356 |
| Example 408 | 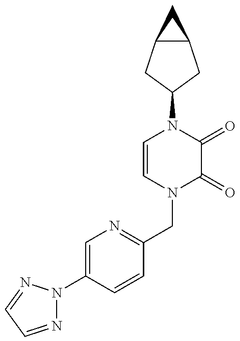 [SMILES: O=c1c(=O)n([C@H]2C[C@H]3C[C@H]3C2)ccn1Cc1ccc(-n2nccn2)cn1] | 1-((5-(2H-1,2,3-triazol-2- yl)pyridin-2-yl)methyl)-4-((cis)- bicyclo[3.1.0]hexan-3-yl)-1,4- dihydropyrazine-2,3-dione | 351 |
| Example 409 | 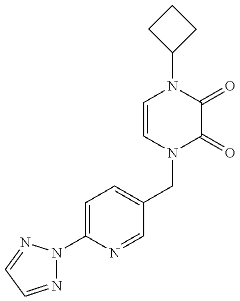 [SMILES: O=c1c(=O)n(C2CCC2)ccn1Cc1ccc(-n2nccn2)nc1] | 1-((6-(2H-1,2,3-triazol-2- yl)pyridin-3-yl)methyl)-4- cyclobutyl-1,4-dihydropyrazine- 2,3-dione | 325 |
| Example 410 | 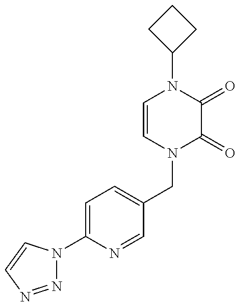 [SMILES: O=c1c(=O)n(C2CCC2)ccn1Cc1ccc(-n2ccnn2)nc1] | 1-((6-(1H-1,2,3-triazol-1- yl)pyridin-3-yl)methyl)-4- cyclobutyl-1,4-dihydropyrazine- 2,3-dione | 325 |
| Example 411 | 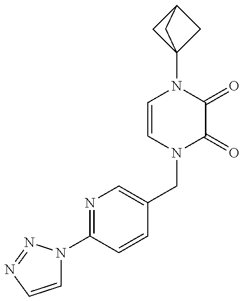 [SMILES: O=c1c(=O)n(C23CC(C2)C3)ccn1Cc1ccc(-n2ccnn2)nc1] | 1-((6-(1H-1,2,3-triazol-1- yl)pyridin-3-yl)methyl)-4- (bicyclo[1.1.1]pentan-1-yl)-1,4- dihydropyrazine-2,3-dione | 337 |
| Example 412 | 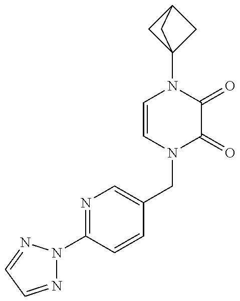 [SMILES: O=c1c(=O)n(C23CC(C2)C3)ccn1Cc1ccc(-n2nccn2)nc1] | 1-((6-(2H-1,2,3-triazol-2- yl)pyridin-3-yl)methyl)-4- (bicyclo[1.1.1]pentan-1-yl)-1,4- dihydropyrazine-2,3-dione | 337 |
| Example 413 | 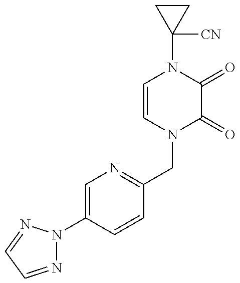 [SMILES: N#CC1(n2ccn(Cc3ccc(-n4nccn4)cn3)c(=O)c2=O)CC1] | 1-(4-((5-(2H-1,2,3-triazol-2- yl)pyridin-2-yl)methyl)-2,3- dioxo-3,4-dihydropyrazin- 1(2H)-yl)cyclopropane-1- carbonitrile | 336 |
| Example 414 | 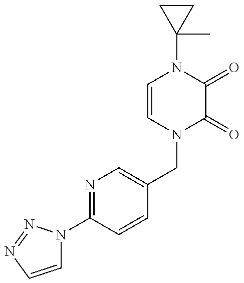 [SMILES: CC1(n2ccn(Cc3ccc(-n4ccnn4)nc3)c(=O)c2=O)CC1] | 1-((6-(1H-1,2,3-triazol-1- yl)pyridin-3-yl)methyl)-4-(1- methylcyclopropyl)-1,4- dihydropyrazine-2,3-dione | 325 |
| Example 415 | 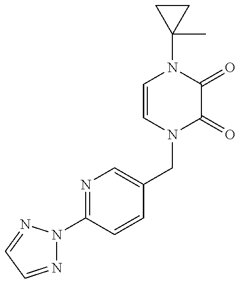 [SMILES: CC1(n2ccn(Cc3ccc(-n4nccn4)nc3)c(=O)c2=O)CC1] | 1-((6-(2H-1,2,3-triazol-2- yl)pyridin-3-yl)methyl)-4-(1- methylcyclopropyl)-1,4- dihydropyrazine-2,3-dione | 325 |

### Example 416

### Preparation of 1-((cis)-bicyclo[3.1.0]hexan-3-yl)-4-((6-(pyridin-3-yl)pyridazin-3-yl)methyl)-1,4-dihydropyrazine-2,3-dione

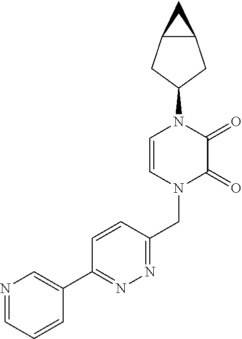 [SMILES: O=c1c(=O)n([C@H]2C[C@H]3C[C@H]3C2)ccn1Cc1ccc(-c2cccnc2)nn1]

### Step A: N-((6-chloropyridazin-3-yl)methyl)prop-2-en-1-amine

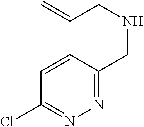

Prop-2-en-1-amine (5.20 mL, 69.2 mmol) was added to a mixture of 3-chloro-6-(chloromethyl)pyridazine hydrochloride (3.0 g, 15 mmol) and potassium carbonate (6.24 g, 45.1 mmol) in DMF (40 mL) at room temperature. The mixture was stirred and heated at 40° C. for 12 hours. The mixture was filtered and concentrated under reduced pressure to afford N-((6-chloropyridazin-3-yl)methyl)prop-2-en-1-amine, which was used without purification in the next step. LC/MS (m/z): 184 (M+H)⁺

### Step B: ethyl 2-(allyl((6-chloropyridazin-3-yl)methyl)amino)-2-oxoacetate

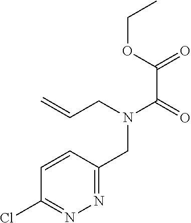 [SMILES: C=CCN(Cc1ccc(Cl)nn1)C(=O)C(=O)OCC]

Ethyl oxalyl chloride (2.52 mL, 22.5 mmol) was added to a solution of N-((6-chloropyridazin-3-yl)methyl)prop-2-en-1-amine (2.76 g, 15.0 mmol) and potassium carbonate (4.15 g, 30.1 mmol) in DMF (20 mL) at 0° C. The mixture was stirred at room temperature for 3 hours. The reaction mixture was quenched with water (400 mL) and extracted with EtOAc (3×100 mL). The organic layers were combined, washed with brine (200 mL), dried over anhydrous Na₂SO₄, filtered, and concentrated under reduced pressure. The residue was purified by silica gel chromatography (eluting ethyl acetate in petroleum ether) to afford ethyl 2-(allyl((6-chloropyridazin-3-yl)methyl)amino)-2-oxoacetate. ¹H NMR (500 MHz, methanol-d₄) δ 7.87-7.68 (m, 2H), 5.96-5.69 (m, 1H), 5.38-5.15 (m, 2H), 4.87 (s, 2H), 4.44-4.28 (m, 2H), 4.14-4.07 (m, 2H), 1.39-1.26 (m, 3H). LC/MS (m/z): 284 (M+H)⁺

### Step C: 3-((N-allyl-2-ethoxy-2-oxoacetamido)methyl)-6-chloropyridazine 1-oxide

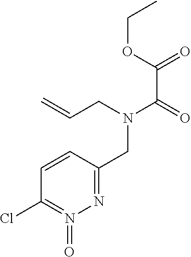 [SMILES: C=CCN(Cc1ccc(Cl)[n+]([O-])n1)C(=O)C(=O)OCC]

meta-Chloroperoxybenzoic acid (0.760 g, 3.52 mmol) was added to a solution of ethyl 2-(allyl((6-chloropyridazin-3-yl)methyl)amino)-2-oxoacetate (1.0 g, 3.5 mmol) in DCM (25 mL) at 0° C. The mixture was warmed to room temperature and stirred for 16 hours. The reaction mixture was quenched with Na₂SO₃ (25 mL) and extracted with EtOAc (3×25 mL). The combined organic layers were washed with brine (25 mL), dried over anhydrous Na₂SO₄, filtered, and concentrated under reduced pressure. The residue was purified by silica gel chromatography (eluting ethyl acetate in petroleum ether) to afford 3-((N-allyl-2-ethoxy-2-oxoacetamido)methyl)-6-chloropyridazine 1-oxide. LC/MS (m/z): 300 (M+H)⁺

### Step D: 6-chloro-3-((2-ethoxy-2-oxo-N-(2-oxoethyl)acetamido)methyl)pyridazine 1-oxide

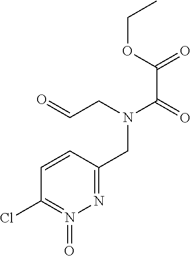 [SMILES: CCOC(=O)C(=O)N(CC=O)Cc1ccc(Cl)[n+]([O-])n1]

Osmium(VIII) oxide (34 mg, 0.13 mmol) was added to a mixture of 3-((N-allyl-2-ethoxy-2-oxoacetamido)methyl)-6-chloropyridazine 1-oxide (0.400 g, 1.33 mmol) and sodium periodate (1.13 g, 5.30 mmol) in water (5 mL) and THE (5 mL) at 0° C. The mixture was warmed to room temperature and stirred for 1.5 hours. The mixture was concentrated under reduced pressure, and the residue was partitioned between water (5 mL) and EtOAc (5 mL). The organic layer was separated, and the aqueous was extracted with EtOAc (3×5 mL). The organic layers were combined, washed with brine (5 mL), dried over anhydrous Na₂SO₄, filtered, and concentrated under reduced pressure to afford 6-chloro-3-((2-ethoxy-2-oxo-N-(2-oxoethyl)acetamido)methyl)pyridazine 1-oxide which was used in the next step without purification. LC/MS (m/z): 302 (M+H)⁺

### Step E: 3-((4-((cis)-bicyclo[3.1.0]hexan-3-yl)-2,3-dioxo-3,4-dihydropyrazin-1(2H)-yl)methyl)-6-chloropyridazine-1-oxide

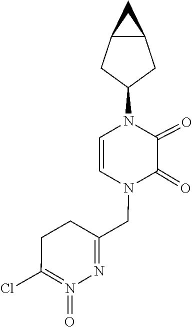 [SMILES: O=c1c(=O)n([C@H]2C[C@H]3C[C@H]3C2)ccn1CC1=N[N+]([O-])=C(Cl)CC1]

Triethylamine (0.069 ml, 0.50 mmol) was added to a mixture of cis-bicyclo[3.1.0]hexan-3-ammonium iodide (112 mg, 0.497 mmol) in DCE (2 mL) and acetic acid (0.5 mL) room temperature. 6-chloro-3-((2-ethoxy-2-oxo-N-(2-oxoethyl)acetamido)methyl)pyridazine-1-oxide (0.150 g, 0.497 mmol) was added to the mixture at room temperature, and the mixture was stirred for 2 days. The mixture was concentrated under reduced pressure, and the residue was partitioned between water (5 mL) and DCM (5 mL). The organic layer was separated, and the aqueous was re-extracted with DCM (3×5 mL). The organic layers were combined, washed with brine (5 mL), dried over anhydrous Na₂SO₄, filtered, and concentrated under reduced pressure. The residue was purified by reverse phase HPLC (eluting acetonitrile in water, with TFA modifier) to afford 3-((4-((cis)-bicyclo[3.1.0]hexan-3-yl)-2,3-dioxo-3,4-dihydropyrazin-1(2H)-yl)methyl)-6-(pyridin-3-yl)pyridazine 1-oxide. LC/MS (m/z): 335 (M+H)⁺

### Step F: 3-((4-((cis)-bicyclo[3.1.0]hexan-3-yl)-2,3-dioxo-3,4-dihydropyrazin-1(2H)-yl)methyl)-6-(pyridin-3-yl)pyridazine 1-oxide

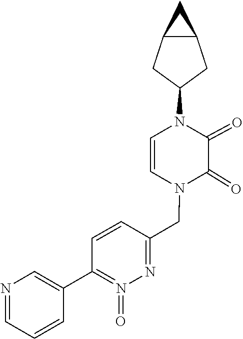 [SMILES: O=c1c(=O)n([C@H]2C[C@H]3C[C@H]3C2)ccn1Cc1ccc(-c2cccnc2)[n+]([O-])n1]

A mixture of 3-((4-(bicyclo[3.1.0]hexan-3-yl)-2,3-dioxo-3,4-dihydropyrazin-1(2H)-yl)methyl)-6-chloropyridazine 1-oxide (10 mg, 0.03 mmol), pyridin-3-ylboronic acid (6 mg, 0.05 mmol), potassium phosphate tribasic (19 mg, 0.090 mmol), and Pd(dtbpf)Cl₂ (5 mg, 7 μmol) in 1,4-dioxane (1 mL) and water (0.2 mL) was degassed and backfilled with N₂ (three times). The mixture was heated to 80° C. and stirred for 16 hours. The mixture was cooled to room temperature and concentrated under reduced pressure. The residue was partitioned between water (10 mL) and DCM (10 mL). The organic layer was separated, and the aqueous was re-extracted with DCM (3×10 mL). The organic layers were combined, washed with brine (10 mL), dried over anhydrous Na₂SO₄, filtered, and concentrated under reduced pressure. The residue was purified by reverse phase HPLC (eluting acetonitrile in water, with TFA modifier) to afford 1-((cis)-bicyclo[3.1.0]hexan-3-yl)-4-((6-(pyridin-3-yl)pyridazin-3-yl)methyl)-1,4-dihydropyrazine-2,3-dione. LC/MS (m/z): 378 (M+H)⁺

### Step G: 1-((cis)-bicyclo[3.1.0]hexan-3-yl)-4-((6-(pyridin-3-yl)pyridazin-3-yl)methyl)-1,4-dihydropyrazine-2,3-dione

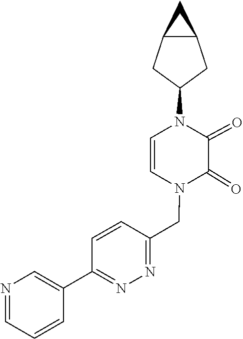 [SMILES: O=c1c(=O)n([C@H]2C[C@H]3C[C@H]3C2)ccn1Cc1ccc(-c2cccnc2)nn1]

A mixture of 6-((4-((cis)-bicyclo[3.1.0]hexan-3-yl)-2,3-dioxo-3,4-dihydropyrazin-1(2H)-yl)methyl)-3-(pyridin-3-yl)pyridazine-1-oxide (5.5 mg, 0.015 mmol), palladium on carbon (10% w/w, 3 mg, 3 μmol) and ammonium formate (3 mg, 0.04 mmol) in MeOH (1 mL) was stirred and heated at 50° C. for 1 hour. The mixture was cooled to room temperature, filtered, and concentrated under reduced pressure. The residue was purified by reverse phase HPLC (eluting acetonitrile in water, with TFA modifier) to afford 1-((cis)-bicyclo[3.1.0]hexan-3-yl)-4-((6-(pyridin-3-yl)pyridazin-3-yl)methyl)-1,4-dihydropyrazine-2,3-dione. ¹H NMR (400 MHz, methanol-d₄) δ 9.41 (s, 1H), 8.89-8.63 (m, 2H), 8.35-8.07 (m, 1H), 7.99-7.75 (m, 2H), 6.87-6.72 (m, 1H), 6.62-6.54 (m, 1H), 5.36 (s, 2H), 5.30-5.21 (m, 1H), 2.43 (ddd, J=4.5, 9.1, 14.0 Hz, 2H), 1.68 (br dd, J=8.6, 13.9 Hz, 2H), 1.40 (br s, 2H), 0.96-0.88 (m, 1H), 0.45 (q, J=4.2 Hz, 1H). LC/MS (m/z): 362 (M+H)⁺

### Example 417

### Preparation of 1-(bicyclo[1.1.1]pentan-1-yl)-4-((5-(3-fluorophenyl)isoxazol-3-yl)methyl)-1,4-dihydropyrazine-2,3-dione

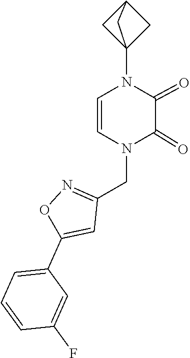 [SMILES: O=c1c(=O)n(C23CC(C2)C3)ccn1Cc1cc(-c2cccc(F)c2)on1]

A mixture of silver(I) fluoride (69 mg, 0.54 mmol), bis(acetonitrile)dichloropalladium(II) (PdCl₂(MeCN)₂) (4 mg, 0.02 mmol), 1,2-bis(diphenylphosphaneyl)benzene (12 mg, 0.027 mmol), 1-fluoro-3-iodobenzene (0.120 g, 0.540 mmol), and 1-(bicyclo[1.1.1]pentan-1-yl)-4-(isoxazol-3-ylmethyl)-1,4-dihydropyrazine-2,3-dione (0.070 g, 0.27 mmol) in DMA (2 mL) was degassed with argon at room temperature. The mixture was stirred and heated at 100° C. for 12 hours. The mixture was cooled to room temperature, filtered, and directly purified by reverse phase HPLC (eluting acetonitrile in water, with TFA modifier) to afford 1-(bicyclo[1.1.1]pentan-1-yl)-4-((5-(3-fluorophenyl)isoxazol-3-yl)methyl)-1,4-dihydropyrazine-2,3-dione. ¹H NMR (400 MHz, methanol-d₄) δ 7.67-7.62 (m, 1H), 7.60-7.54 (m, 1H), 7.54-7.46 (m, 1H), 7.23 (dt, J=1.8, 8.5 Hz, 1H), 6.87 (s, 1H), 6.58 (d, J=6.3 Hz, 1H), 6.49 (d, J=6.3 Hz, 1H), 5.11 (s, 2H), 2.63 (s, 1H), 2.33 (s, 6H). LC/MS (m/z): 354 (M+H)⁺

Examples shown in Example Table 18 below, were or may be prepared according to procedures analogous to those outlined in Example 417 above using the appropriate starting materials, described in the Preparations or Intermediates above, or as obtained from commercial sources.

Example Table 18

| Example | Structure | Name | Mass [M + H]⁺ |
|---|---|---|---|
| Example 418 | 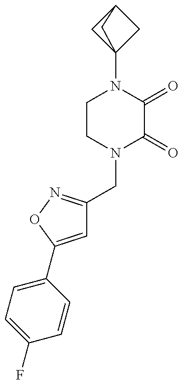 [SMILES: O=C1C(=O)N(C23CC(C2)C3)CCN1Cc1cc(-c2ccc(F)cc2)on1] | 1-(bicyclo[1.1.1]pentan-1- yl)-4-((5-(4- fluorophenyl)isoxazol-3- yl)methyl)piperazine-2,3- dione | 356 |
| Example 419 | 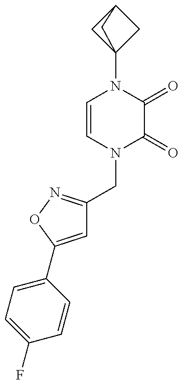 [SMILES: O=c1c(=O)n(C23CC(C2)C3)ccn1Cc1cc(-c2ccc(F)cc2)on1] | 1-(bicyclo[1.1.1]pentan-1- yl)-4-((5-(4- fluorophenyl)isoxazol-3- yl)methyl)-1,4- dihydropyrazine-2,3-dione | 354 |
| Example 420 | 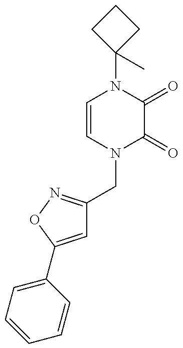 [SMILES: CC1(n2ccn(Cc3cc(-c4ccccc4)on3)c(=O)c2=O)CCC1] | 1-(1-methylcyclobutyl)-4- ((5-phenylisoxazol-3- yl)methyl)-1,4- dihydropyrazine-2,3-dione | 338 |
| Example 421 | 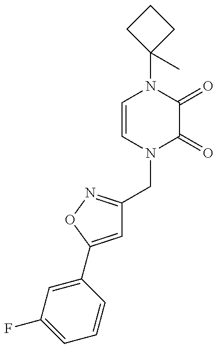 [SMILES: CC1(n2ccn(Cc3cc(-c4cccc(F)c4)on3)c(=O)c2=O)CCC1] | 1-((5-(3- fluorophenyl)isoxazol-3- yl)methyl)-4-(1- methylcyclobutyl)-1,4- dihydropyrazine-2,3-dione | 356 |
| Example 422 | 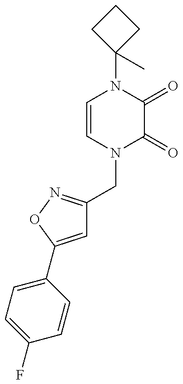 [SMILES: CC1(n2ccn(Cc3cc(-c4ccc(F)cc4)on3)c(=O)c2=O)CCC1] | 1-((5-(4- fluorophenyl)isoxazol-3- yl)methyl)-4-(1- methylcyclobutyl)-1,4- dihydropyrazine-2,3-dione | 356 |
| Example 423 | 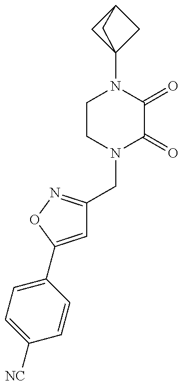 [SMILES: N#Cc1ccc(-c2cc(CN3CCN(C45CC(C4)C5)C(=O)C3=O)no2)cc1] | 4-(3-((4- (bicyclo[1.1.1]pentan-1-yl)- 2,3-dioxopiperazin-1- yl)methyl)isoxazol-5- yl)benzonitrile | 363 |
| Example 424 | 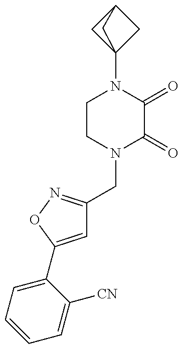 [SMILES: N#Cc1ccccc1-c1cc(CN2CCN(C34CC(C3)C4)C(=O)C2=O)no1] | 2-(3-((4- (bicyclo[1.1.1]pentan-1-yl)- 2,3-dioxopiperazin-1- yl)methyl)isoxazol-5- yl)benzonitrile | 363 |

### Example 425

### Preparation of 1-cyclobutyl-4-((5-(3-fluorophenyl)thiazol-2-yl)methyl)piperazine-2,3-dione

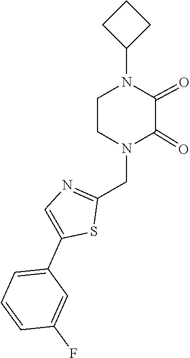 [SMILES: O=C1C(=O)N(C2CCC2)CCN1Cc1ncc(-c2cccc(F)c2)s1]

A mixture of 1-cyclobutyl-4-(thiazol-2-ylmethyl)piperazine-2,3-dione (0.100 g, 0.377 mmol), potassium carbonate (104 mg, 0.754 mmol), pivalic acid (16 mg, 0.16 mmol), cataCXium A® (14 mg, 0.039 mmol), palladium(II) acetate (9 mg, 0.04 mmol), and 1-fluoro-3-iodobenzene (126 mg, 0.565 mmol) in toluene (2 mL) was degassed with nitrogen for 5 minutes at room temperature. The mixture was stirred and heated at 100° C. for 12 hours. The mixture was cooled to room temperature, filtered, and concentrated under reduced pressure. The residue was purified by reverse phase HPLC (eluting acetonitrile in water, with TFA modifier) to afford 1-cyclobutyl-4-((5-(3-fluorophenyl)thiazol-2-yl)methyl)piperazine-2,3-dione. ¹H NMR (500 MHz, methanol-d₄) δ 8.04 (s, 1H), 7.46-7.42 (m, 2H), 7.41-7.36 (m, 1H), 7.14-7.05 (m, 1H), 4.95 (s, 2H), 4.87-4.79 (m, 1H), 3.79-3.73 (m, 2H), 3.73-3.66 (m, 2H), 2.30-2.22 (m, 2H), 2.22-2.15 (m, 2H), 1.80-1.73 (m, 2H). LC/MS (m/z): 360 (M+H)⁺

Examples shown in Example Table 19 below, were or may be prepared according to procedures analogous to those outlined in Example 425 above using the appropriate starting materials, described in the Preparations or Intermediates above, or as obtained from commercial sources.

Example Table 19

| Example | Structure | Name | Mass [M + H]⁺ |
|---|---|---|---|
| Example 426 | 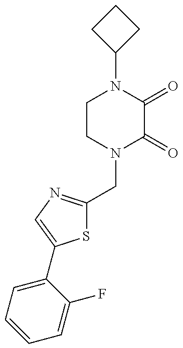 [SMILES: O=C1C(=O)N(C2CCC2)CCN1Cc1ncc(-c2ccccc2F)s1] | 1-cyclobutyl-4-((5-(2- fluorophenyl)thiazol-2- yl)methyl)piperazine-2,3- dione | 360 |

### Example 427

### Preparation of 1-(bicyclo[1.1.1]pentan-1-yl)-4-((1-(pyridin-2-yl)-1H-1,2,3-triazol-4-yl)methyl)-1,4-dihydropyrazine-2,3-dione

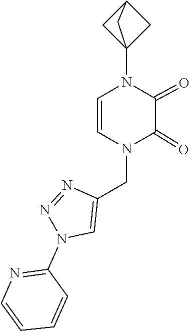 [SMILES: O=c1c(=O)n(C23CC(C2)C3)ccn1Cc1cn(-c2ccccn2)nn1]

A mixture of copper(II) sulfate (4 mg, 0.02 mmol) and (+)-sodium L-ascorbate (5 mg, 0.02 mmol) in water (0.1 mL) was added to a mixture of 1-(bicyclo[1.1.1]pentan-1-yl)-4-(prop-2-yn-1-yl)-1,4-dihydropyrazine-2,3-dione (0.050 g, 0.23 mmol) and 2-azidopyridine (56 mg, 0.46 mmol) in t-BuOH (0.9 mL) at room temperature. The mixture was stirred and heated at 50° C. for 16 hours. The mixture was filtered and the filtrate was purified by reverse phase HPLC (eluting acetonitrile in water, with ammonium bicarbonate modifier) to afford 1-(bicyclo[1.1.1]pentan-1-yl)-4-((1-(pyridin-2-yl)-1H-1,2,3-triazol-4-yl)methyl)-1,4-dihydropyrazine-2,3-dione. ¹H NMR (500 MHz, chloroform-d) δ 8.75 (br s, 1H), 8.51 (br s, 1H), 8.16 (br s, 1H), 7.92 (t, J=7.78 Hz, 1H), 7.38 (br s, 1H), 6.46 (d, J=6.10 Hz, 1H), 6.16 (d, J=5.95 Hz, 1H), 5.12 (s, 2H), 2.62 (s, 1H), 2.29 (s, 6H). LC/MS (m/z): 337 (M+H)⁺

Examples shown in Example Table 20 below, were or may be prepared according to procedures analogous to those outlined in Example 427 above using the appropriate starting materials, described in the Preparations or Intermediates above, or as obtained from commercial sources.

Example Table 20

| Example | Structure | Name | Mass [M + H]⁺ |
|---|---|---|---|
| Example 428 | 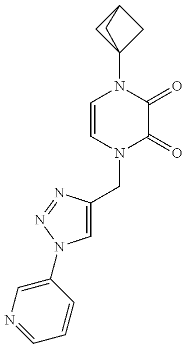 [SMILES: O=c1c(=O)n(C23CC(C2)C3)ccn1Cc1cn(-c2cccnc2)nn1] | 1-(bicyclo[1.1.1]pentan-1-yl)- 4-((1-(pyridin-3-yl)-1H-1,2,3- triazol-4-yl)methyl)-1,4- dihydropyrazine-2,3-dione | 337 |
| Example 429 | 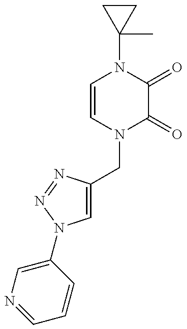 [SMILES: CC1(n2ccn(Cc3cn(-c4cccnc4)nn3)c(=O)c2=O)CC1] | 1-(1-methylcyclopropyl)-4- ((1-(pyridin-3-yl)-1H-1,2,3- triazol-4-yl)methyl)-1,4- dihydropyrazine-2,3-dione | 325 |
| Example 430 | 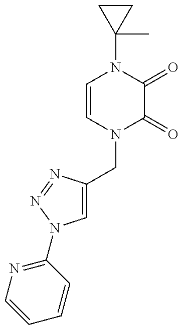 [SMILES: CC1(n2ccn(Cc3cn(-c4ccccn4)nn3)c(=O)c2=O)CC1] | 1-(1-methylcyclopropyl)-4- ((1-(pyridin-2-yl)-1H-1,2,3- triazol-4-yl)methyl)-1,4- dihydropyrazine-2,3-dione | 325 |
| Example 431 | 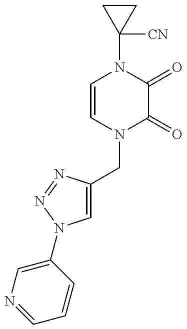 [SMILES: N#CC1(n2ccn(Cc3cn(-c4cccnc4)nn3)c(=O)c2=O)CC1] | 1-(2,3-dioxo-4-((1-(pyridin-3- yl)-1H-1,2,3-triazol-4- yl)methyl)-3,4- dihydropyrazin-1(2H)- yl)cyclopropane-1- carbonitrile | 336 |

### Example 432

### Preparation of 1-((4-bromo-5-(pyri din-2-yl)isoxazol-3-yl)methyl)-4-cyclobutylpiperazine-2,3-di one

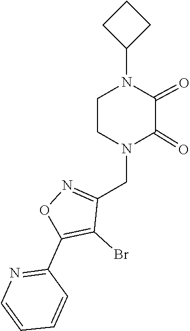 [SMILES: O=C1C(=O)N(C2CCC2)CCN1Cc1noc(-c2ccccn2)c1Br]

NBS (327 mg, 1.84 mmol) was added to a mixture of 1-cyclobutyl-4-((5-(pyridin-2-yl)isoxazol-3-yl)methyl)piperazine-2,3-dione (0.200 g, 0.613 mmol) in DMF (2 ml) at room temperature. The mixture was stirred and heated to 100° C. for 12 hours. The mixture was concentrated under reduced pressure, and the residue was purified by reverse phase HPLC (eluting acetonitrile in water, with TFA modifier) to afford 1-((4-bromo-5-(pyridin-2-yl)isoxazol-3-yl)methyl)-4-cyclobutylpiperazine-2,3-dione. ¹H NMR (500 MHz, methanol-d₄) δ 8.75 (d, J=4.7 Hz, 1H), 8.13 (d, J=7.9 Hz, 1H), 8.06-8.00 (m, 1H), 7.58-7.56 (m, 1H), 4.91-4.89 (m, 2H), 4.86-4.83 (m, 1H), 3.72 (s, 4H), 2.31-2.24 (m, 2H), 2.24-2.16 (m, 2H), 1.81-1.73 (m, 2H). LC/MS (m/z): 405, 407 (M+H)⁺

### Example 433

### Preparation of 1-((6-phenylpyridazin-3-yl)methyl)-4-(pyridin-2-yl)piperazine-2,3-dione

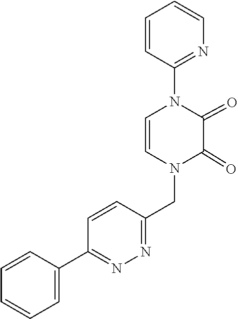 [SMILES: O=c1c(=O)n(-c2ccccn2)ccn1Cc1ccc(-c2ccccc2)nn1]

A mixture of 1-((6-phenylpyridazin-3-yl)methyl)piperazine-2,3-dione (0.050 g, 0.18 mmol), 2-iodopyridine (55 mg, 0.27 mmol), dimethylglycine (11 mg, 0.11 mmol), copper (I) iodide (10 mg, 0.053 mmol), and K₂CO₃ (73 mg, 0.53 mmol) in DMSO (1 mL) was stirred and heated at 100° C. for 4 hours. The mixture was filtered and the filtrate was directly purified by reverse phase HPLC (eluting acetonitrile in water, with TFA modifier) to afford 1-((6-phenylpyridazin-3-yl)methyl)-4-(pyridin-2-yl)piperazine-2,3-dione. ¹H NMR (400 MHz, chloroform-d) δ 8.45 (d, J=3.9 Hz, 1H), 8.11 (d, J=8.6 Hz, 1H), 8.07-8.00 (m, 3H), 7.99-7.92 (m, 1H), 7.83-7.76 (m, 1H), 7.59-7.54 (m, 3H), 7.19 (dd, J=7.2, 4.9 Hz, 1H), 5.13 (s, 2H), 4.40 (br s, 2H), 4.00 (br s, 2H). LC/MS (m/z): 360 (M+H)⁺

### Example 434

### Preparation of 1-((6-(2-fluorophenyl)pyridazin-3-yl)methyl)-4-(3-hydroxycyclobutyl)piperazine-2,3-dione

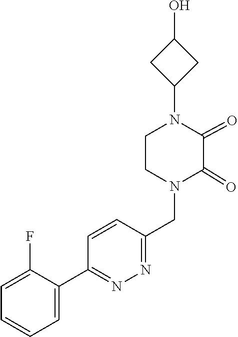 [SMILES: O=C1C(=O)N(C2CC(O)C2)CCN1Cc1ccc(-c2ccccc2F)nn1]

### Step A: 1-((6-(2-fluorophenyl)pyridazin-3-yl)methyl)-4-(3-oxocyclobutyl)piperazine-2,3-dione

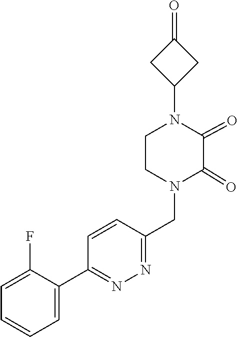 [SMILES: O=C1CC(N2CCN(Cc3ccc(-c4ccccc4F)nn3)C(=O)C2=O)C1]

A mixture of 1-((6-(2-fluorophenyl)pyridazin-3-yl)methyl)piperazine-2,3-dione (10 mg, 0.03 mmol), 3-bromocyclobutan-1-one (6 mg, 0.04 mmol), and Cs₂CO₃ (22 mg, 0.068 mmol) in DMF (1 mL) was stirred and heated at 60° C. for 12 hours. The mixture was concentrated under reduced pressure and the residue was partitioned between water (10 mL) and DCM (10 mL). The organic layer was separated and the aqueous was re-extracted with DCM (3×10 mL). The organic layers were combined, washed with brine (10 mL), dried over anhydrous sodium sulfate, filtered, and concentrated under reduced pressure afford 1-((6-(2-fluorophenyl)pyridazin-3-yl)methyl)-4-(3-oxocyclobutyl)piperazine-2,3-dione, which was used without purification in the next step. LC/MS (m/z): 369 (M+H)⁺

### Step B: 1-((6-(2-fluorophenyl)pyridazin-3-yl)methyl)-4-(3-hydroxycyclobutyl)piperazine-2,3-dione

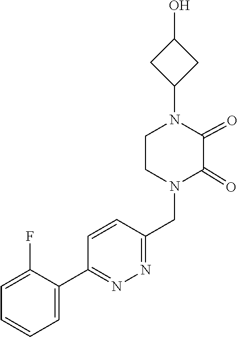 [SMILES: O=C1C(=O)N(C2CC(O)C2)CCN1Cc1ccc(-c2ccccc2F)nn1]

A mixture of 1-((6-(2-fluorophenyl)pyridazin-3-yl)methyl)-4-(3-oxocyclobutyl)piperazine-2,3-dione (0.100 g, 0.271 mmol) and NaBH₄ (10 mg, 0.3 mmol) in MeOH (4 ml) was stirred at room temperature for 12 hours. The mixture was quenched with water (1 mL). The mixture was filtered and the filtrate was concentrated under reduced pressure. The residue was purified by reverse phase HPLC (eluting acetonitrile in water, with TFA modifier) to afford 1-((6-(2-fluorophenyl)pyridazin-3-yl)methyl)-4-(3-hydroxycyclobutyl)piperazine-2,3-dione. ¹H NMR (500 MHz, methanol-d₄) δ 8.07 (dd, J=1.8, 8.7 Hz, 1H), 7.95 (br t, J 7.2 Hz, 1H), 7.82 (d, J=8.7 Hz, 1H), 7.62-7.54 (m, 1H), 7.38 (t, J=7.6 Hz, 1H), 7.31 (dd, J=8.4, 11.1 Hz, 1H), 5.04 (s, 2H), 4.45-4.35 (m, 1H), 4.02 (quin, J=7.1 Hz, 1H), 3.91-3.69 (m, 4H), 2.69-2.54 (m, 2H), 2.16-2.04 (m, 2H). LC/MS (m/z): 371 (M+H)⁺

### Examples 435A and 435B

### Preparation of 1-cyclobutyl-4-((6-phenylpyridazin-3-yl)methyl)hexahydro-1H-cyclopenta[b]pyrazine-2,3-dione (Trans stereoisomer; S,S or R,R) and 1-cyclobutyl-4-((6-phenylpyridazin-3-yl)methyl)hexahydro-1H-cyclopenta[b]pyrazine-2,3-dione (Cis stereoisomer; S,R or R,S)

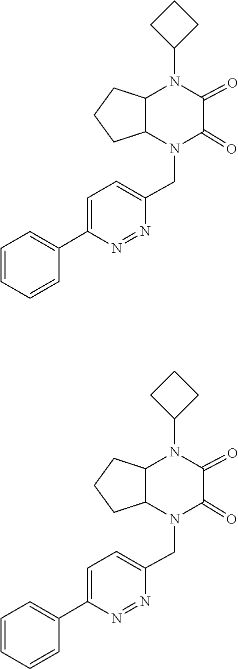 [SMILES: O=C1C(=O)N(C2CCC2)C2CCCC2N1Cc1ccc(-c2ccccc2)nn1 ; O=C1C(=O)N(C2CCC2)C2CCCC2N1Cc1ccc(-c2ccccc2)nn1]

3-(Chloromethyl)-6-phenylpyridazine (44 mg, 0.22 mmol) was added to a mixture of 1-cyclobutylhexahydro-1H-cyclopenta[b]pyrazine-2,3-dione (45 mg, 0.22 mmol) and Cs₂CO₃ (141 mg, 0.432 mmol) in DMF (2 mL). The mixture was stirred and heated to 60° C. for 2 hours. The reaction mixture was filtered and the residue was purified by reverse phase HPLC (eluting acetonitrile in water, with TFA modifier) to give 1-cyclobutyl-4-((6-phenylpyridazin-3-yl)methyl)hexahydro-1H-cyclopenta[b]pyrazine-2,3-dione as a mixture of four isomers. The residue was resolved by chiral SFC (Column: Daicel Chiralpak AD, 250×30 mm, 10 μm; eluting 45% [0.1% ammonia in water] in isopropanol) to afford the product in two fractions, each a mixture of two stereoisomers. The second fraction was further resolved by chiral SFC (Column: Daicel Chiralpak AD, 250×30 mm, 10 μm; eluting 45% [0.1% ammonia in water] in isopropanol) to afford:

Peak 1: Example 435A: (S,S or R,R)-1-cyclobutyl-4-((6-phenylpyridazin-3-yl)methyl)hexahydro-1H-cyclopenta[b]pyrazine-2,3-dione. ¹H NMR (500 MHz, chloroform-d) δ 8.08 (dd, J=1.5, 7.6 Hz, 2H), 7.89-7.84 (m, 1H), 7.82-7.79 (m, 1H), 7.56-7.47 (m, 3H), 5.15 (d, J=14.5 Hz, 1H), 4.90 (d, J=14.3 Hz, 1H), 4.19 (q, J=5.4 Hz, 1H), 3.99 (q, J=6.2 Hz, 1H), 3.59 (dd, J=7.3, 14.2 Hz, 1H), 3.20 (dd, J=7.0, 14.2 Hz, 1H), 2.20-2.08 (m, 1H), 1.96-1.88 (m, 1H), 1.74-1.65 (m, 2H), 1.03-0.97 (m, 1H), 0.61-0.46 (m, 2H), 0.40-0.23 (m, 2H). LC/MS (m/z): 377 (M+H)⁺

Peak 2: Example 435B: (S,R or S,R)-1-cyclobutyl-4-((6-phenylpyridazin-3-yl)methyl)hexahydro-1H-cyclopenta[b]pyrazine-2,3-dione. ¹H NMR (500 MHz, chloroform-d) δ 8.14-8.01 (m, 2H), 7.88-7.82 (m, 1H), 7.81-7.74 (m, 1H), 7.56-7.46 (m, 3H), 5.17 (d, J=14.6 Hz, 1H), 4.88 (d, J=14.6 Hz, 1H), 4.73-4.62 (m, 1H), 4.16 (dt, J=2.2, 5.1 Hz, 1H), 3.95-3.82 (m, 1H), 2.51-2.39 (m, 1H), 2.32-2.24 (m, 1H), 2.24-2.18 (m, 1H), 2.17-2.04 (m, 2H), 1.96-1.86 (m, 1H), 1.78-1.66 (m, 3H), 1.63-1.55 (m, 1H). LC/MS (m/z): 377 (M+H)⁺

### Example 436

### Preparation of 2-(5-((4-(bicyclo[1.1.1]pentan-1-yl)-2,3-dioxopiperazin-1-yl)methyl)-1,3,4-thiadiazol-2-yl)benzonitrile

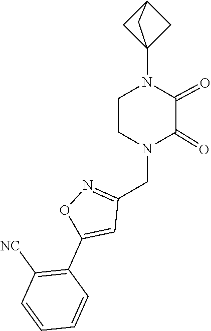 [SMILES: N#Cc1ccccc1-c1cc(CN2CCN(C34CC(C3)C4)C(=O)C2=O)no1]

A mixture of 1-(bicyclo[1.1.1]pentan-1-yl)-4-((5-(2-chlorophenyl)-1,3,4-thiadiazol-2-yl)methyl)piperazine-2,3-dione (0.090 g, 0.23 mmol), potassium ferrocyanide trihydrate (49 mg, 0.12 mmol), and Brettphos Pd G3 (42 mg, 0.046 mmol)) in DMA (1 mL) and water (0.333 mL) was degassed and backfilled with nitrogen (3×). The mixture was stirred and heated at 100° C. for 4 hours. The mixture was filtered and the filtrate was directly purified by reverse phase HPLC (eluting acetonitrile in water, with TFA modifier) to afford 2-(5-((4-(bicyclo[1.1.1]pentan-1-yl)-2,3-dioxopiperazin-1-yl)methyl)-1,3,4-thiadiazol-2-yl)benzonitrile. ¹H NMR (400 MHz, CD30D) S 8.04 (d, J=7.6 Hz, 1H), 7.95 (d, J=8.0 Hz, 1H), 7.85-7.79 (m, 1H), 7.75-7.68 (m, 1H), 5.11 (s, 2H), 3.81-3.76 (m, 2H), 3.64-3.59 (m, 2H), 2.49 (s, 1H), 2.19 (s, 6H). LC/MS (m/z): 380 (M+H)⁺

### Example 437

### Preparation of (R or S)-1-cyclobutyl-5-methyl-4-((5-phenyl-1,3,4-thiadiazol-2-yl)methyl)piperazine-2,3-dione

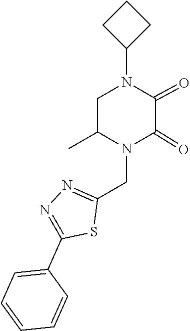 [SMILES: CC1CN(C2CCC2)C(=O)C(=O)N1Cc1nnc(-c2ccccc2)s1]

(rac)-1-Cyclobutyl-5-methyl-4-((5-phenyl-1,3,4-thiadiazol-2-yl)methyl)piperazine-2,3-dione (0.050 g, 0.106 mmol) was resolved by chiral SFC (Column: CCO F4 (21×250 mm, 5 μm); eluting 30% MeOH (with 0.1% ammonium hydroxide) in CO₂) to afford (R or S)-1-cyclobutyl-5-methyl-4-((5-phenyl-1,3,4-thiadiazol-2-yl)methyl)piperazine-2,3-dione as the first eluting peak.

Peak 1: (R or S)-1-cyclobutyl-5-methyl-4-((5-phenyl-1,3,4-thiadiazol-2-yl)methyl)piperazine-2,3-dione (tᵣ=5.5 minutes): ¹H NMR (600 MHz, DMSO-d₆) δ 8.00-7.96 (m, 2H), 7.60-7.54 (m, 3H), 5.16 (d, J=15.7 Hz, 1H), 4.88 (d, J=15.7 Hz, 1H), 4.82 (p, J=8.9 Hz, 1H), 3.98-3.92 (m, 1H), 3.72 (dd, J=13.3, 3.9 Hz, 1H), 3.53 (dd, J=13.3, 2.6 Hz, 1H), 2.20-2.10 (m, 2H), 2.09-1.99 (m, 2H), 1.70-1.64 (m, 2H), 1.26 (d, J=6.5 Hz, 3H). LC/MS (m/z): 357 (M+H)⁺

### Example 438

### Preparation of (R,S or S,R)-1-(3-hydroxycyclopentyl)-4-((5-phenyl-1,3,4-thiadiazol-2-yl)methyl)piperazine-2,3-dione

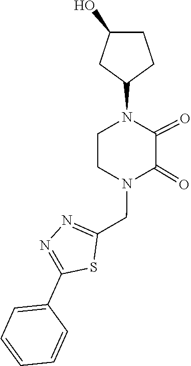

A mixture 1-((5-bromo-1,3,4-thiadiazol-2-yl)methyl)-4-(3-hydroxycyclopentyl)piperazine-2,3-dione (0.100 g, 0.266 mmol), phenylboronic acid (49 mg, 0.40 mmol), potassium phosphate tribasic (0.170 g, 0.799 mmol), and [1,1′-bis(diphenylphosphino)ferrocene]dichloropalladium (II) (20 mg, 0.027 mmol) in 1,4-dioxane (2 mL) was stirred and heated at 100° C. for 12 hours. The reaction mixture was filtered and concentrated under reduced pressure. The residue was purified by reverse phase HPLC (eluting acetonitrile in water, with TFA modifier) to afford the product as a mixture of stereoisomers. The mixture was resolved by chiral SFC (Column: Phenomenex-Cellulose-2 (250 mm×30 mm, 5 μm); eluting 5-40% ethanol (with 0.1% ammonium hydroxide modifier) in CO₂) to afford (R,S or S,R)-1-(3-hydroxycyclopentyl)-4-((5-phenyl-1,3,4-thiadiazol-2-yl)methyl)piperazine-2,3-dione as the first eluting peak.

Peak 1: (R,S or S,R)-1-(3-hydroxycyclopentyl)-4-((5-phenyl-1,3,4-thiadiazol-2-yl)methyl)piperazine-2,3-dione (tᵣ=3.67 minutes). ¹H NMR (400 MHz, CDCl₃) δ 7.95-7.87 (m, 2H), 7.54-7.41 (m, 3H), 5.04 (s, 2H), 4.88-4.73 (m, 1H), 4.35-4.30 (m, 1H), 3.75-3.62 (m, 4H), 2.28-2.19 (m, 1H), 1.98-1.73 (m, 5H). LC/MS (m/z): 373 (M+H)⁺

### Example 439

### Preparation of 1-(bicyclo[1.1.1]pentan-1-yl)-4-((1-phenyl-1H-1,2,3-triazol-4-yl)methyl)piperazine-2,3-dione

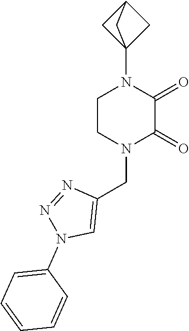 [SMILES: O=C1C(=O)N(C23CC(C2)C3)CCN1Cc1cn(-c2ccccc2)nn1]

### Step A: 1-(bicyclo[1.1.1]pentan-1-yl)-4-(prop-2-yn-1-yl)piperazine-2,3-dione

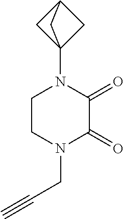 [SMILES: C#CCN1CCN(C23CC(C2)C3)C(=O)C1=O]

A mixture of 1-(bicyclo[1.1.1]pentan-1-yl)piperazine-2,3-dione (750 mg, 4.16 mmol), 3-bromoprop-1-yne (0.673 mL, 6.24 mmol) and Cs₂CO₃ (2030 mg, 6.24 mmol) in DMF (10 mL) was stirred and heated at 60° C. for 16 hours. The mixture was diluted with EtOAc (30 mL) and filtered. The filtrate was concentrated under reduced pressure. The residue was purified by silica gel chromatography (eluting ethyl acetate in petroleum ether) to afford 1-(bicyclo[1.1.1]pentan-1-yl)-4-(prop-2-yn-1-yl)piperazine-2,3-dione. ¹H NMR (400 MHz, chloroform-d) δ 4.34 (s, 2H), 3.68-3.61 (m, 2H), 3.56-3.49 (m, 2H), 2.55-2.52 (m, 1H), 2.31-2.27 (m, 1H), 2.20 (s, 6H). LC/MS (m/z): 219 (M+H)⁺

### Step B: 1-(bicyclo[1.1.1]pentan-1-yl)-4-((1-phenyl-1H-1,2,3-triazol-4-yl)methyl)piperazine-2,3-dione

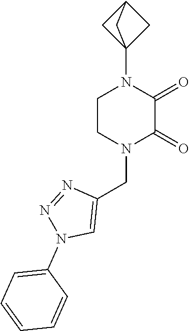 [SMILES: O=C1C(=O)N(C23CC(C2)C3)CCN1Cc1cn(-c2ccccc2)nn1]

A mixture of copper (II) sulfate (3.7 mg, 0.023 mmol) and (+)-sodium L-ascorbate (4.5 mg, 0.023 mmol) in water (0.1 mL) was added to a mixture of 1-(bicyclo[1.1.1]pentan-1-yl)-4-(prop-2-yn-1-yl)piperazine-2,3-dione (0.050 g, 0.23 mmol) and azidobenzene (55 mg, 0.46 mmol) in t-BuOH (0.9 ml) at room temperature. The mixture was stirred and heated at 50° C. for 16 hours. The mixture was filtered and the filtrate was purified by reverse phase HPLC (eluting acetonitrile in water, with TFA modifier) to afford 1-(bicyclo[1.1.1]pentan-1-yl)-4-((1-phenyl-1H-1,2,3-triazol-4-yl)methyl)piperazine-2,3-dione. ¹H NMR (500 MHz, chloroform-d) δ 8.16 (s, 1H), 7.73 (d, J=7.78 Hz, 2H), 7.56-7.51 (m, 2H), 7.48-7.43 (m, 1H), 4.79 (s, 2H), 3.78 (br s, 2H), 3.50 (br s, 2H), 2.52 (s, 1H), 2.18 (s, 6H). LC/MS (m/z): 338 (M+H)⁺

### Examples 440A, 440B, 440C and 440D

### Preparation of (R or S, R or S)-1-((5-phenyl-1,3,4-thiadiazol-2-yl)methyl)-4-(3-phenylcyclopentyl)piperazine-2,3-dione, (R or S, R or S)-1-((5-phenyl-1,3,4-thiadiazol-2-yl)methyl)-4-(3-phenylcyclopentyl)piperazine-2,3-dione, (R or S, R or S)-1-((5-phenyl-1,3,4-thiadiazol-2-yl)methyl)-4-(3-phenylcyclopentyl)piperazine-2,3-dione, and (R or S, R or S)-1-((5-phenyl-1,3,4-thiadiazol-2-yl)methyl)-4-(3-phenylcyclopentyl)piperazine-2,3-dione

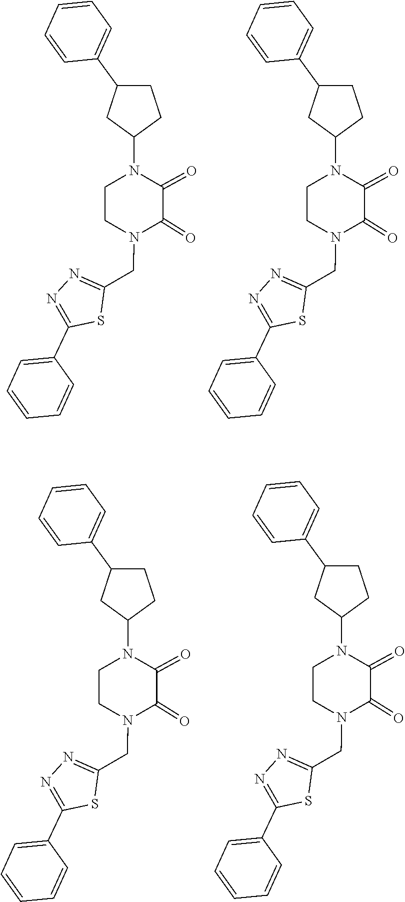 [SMILES: O=C1C(=O)N(C2CCC(c3ccccc3)C2)CCN1Cc1nnc(-c2ccccc2)s1 ; O=C1C(=O)N(C2CCC(c3ccccc3)C2)CCN1Cc1nnc(-c2ccccc2)s1 ; O=C1C(=O)N(C2CCC(c3ccccc3)C2)CCN1Cc1nnc(-c2ccccc2)s1 ; O=C1C(=O)N(C2CCC(c3ccccc3)C2)CCN1Cc1nnc(-c2ccccc2)s1]

Sodium triacetoxyborohydride (636 mg, 3.00 mmol) was added to a mixture of ethyl 2-oxo-2-((2-oxoethyl)((5-phenyl-1,3,4-thiadiazol-2-yl)methyl)amino)acetate (0.500 g, 1.50 mmol) and 3-phenylcyclopentan-1-amine oxalate (377 mg, 1.50 mmol) in DCM (10 ml) at 0° C. The mixture was stirred at room temperature for 1 hour. The mixture was filtered and the filtrate was concentrated under reduced pressure. The residue was purified by reverse phase HPLC (eluting acetonitrile in water, with TFA modifier) to afford the product as a mixture of stereoisomers. The mixture was resolved by chiral SFC (Column: Daicel-Chiralcel-OJ (100×4.6 mm, 3 μm); eluting 60% methanol (with 0.1% ammonium hydroxide modifier) in CO₂) to afford:

Peak 1: Example 440A (R or S, R or S)-1-((5-phenyl-1,3,4-thiadiazol-2-yl)methyl)-4-(3-phenylcyclopentyl)piperazine-2,3-dione (tᵣ=3.50 minutes). ¹H NMR (500 MHz, chloroform-d) δ 8.07-7.89 (m, 2H), 7.68-7.49 (m, 3H), 7.38-7.32 (m, 2H), 7.28-7.21 (m, 3H), 5.29-5.17 (m, 1H), 5.12 (s, 2H), 3.82 (br t, J=5.42 Hz, 2H), 3.68-3.52 (m, 2H), 3.37-3.16 (m, 1H), 2.30-2.18 (m, 2H), 2.16-1.99 (m, 2H), 1.74-1.68 (m, 2H). LC/MS (m/z): 433 (M+H)⁺

Peak 2: Example 440B (R or S, R or S)-1-((5-phenyl-1,3,4-thiadiazol-2-yl)methyl)-4-(3-phenylcyclopentyl)piperazine-2,3-dione (tᵣ=3.87 minutes). ¹H NMR (500 MHz, chloroform-d) δ 7.97 (br d, J=6.56 Hz, 2H), 7.58-7.46 (m, 3H), 7.33 (q, J=7.53 Hz, 2H), 7.28-7.22 (m, 3H), 5.20-5.11 (m, 1H), 5.10 (d, J=1.68 Hz, 2H), 3.86-3.71 (m, 2H), 3.58 (br t, J=5.72 Hz, 2H), 3.24-3.07 (m, 1H), 2.34 (td, J=6.43, 12.32 Hz, 1H), 2.26-2.08 (m, 2H), 1.91-1.77 (m, 3H). LC/MS (m/z): 433 (M+H)⁺

Peak 3: Example 440C (R or S, R or S)-1-((5-phenyl-1,3,4-thiadiazol-2-yl)methyl)-4-(3-phenylcyclopentyl)piperazine-2,3-dione (tᵣ=6.90 minutes). ¹H NMR (500 MHz, chloroform-d) δ 7.98 (br d, J=6.56 Hz, 2H), 7.56-7.50 (m, 3H), 7.37-7.31 (m, 2H), 7.25 (br d, J=6.71 Hz, 3H), 5.28-5.20 (m, 1H), 5.12 (s, 2H), 3.83 (br t, J=5.65 Hz, 2H), 3.67-3.58 (m, 2H), 3.31-3.19 (m, 1H), 2.28-2.17 (m, 2H), 2.16-2.06 (m, 2H), 1.78-1.69 (m, 2H). LC/MS (m/z): 433 (M+H)⁺

Peak 4: Example 440D (R or S, R or S)-1-((5-phenyl-1,3,4-thiadiazol-2-yl)methyl)-4-(3-phenylcyclopentyl)piperazine-2,3-dione (tᵣ=8.13 minutes). 7.97 (br d, J=6.71 Hz, 2H), 7.56-7.49 (m, 3H), 7.36-7.31 (m, 2H), 7.25 (br d, J=7.63 Hz, 3H), 5.18-5.12 (m, 1H), 5.11 (s, 2H), 3.79 (br d, J=3.36 Hz, 2H), 3.57 (br t, J=5.49 Hz, 2H), 3.25-3.09 (m, 1H), 2.39-2.10 (m, 4H), 1.91-1.73 (m, 2H). LC/MS (m/z): 433 (M+H)⁺

### Assay

### IL4I1 Enzymatic Assay

Interleukin 4 inducible protein 1 (IL4I1) is an L-amino oxidase that catalyzes the oxidation of aromatic residues (Phe, Trp and Tyr): L-amino acid+H₂O+O₂→2-oxo acid+NH₃+H₂O₂. Equal molar of H₂O₂ and the corresponding alpha-ketoacid are produced when IL4I1 and substrate are added. In this assay, the hydrogen peroxide generated by IL4I1 is then detected through a coupled reaction with Amplex® Red (10-acetyl-3,7-dihydroxyphenoxazine) and horseradish peroxidase (HRP) to produce resorufin product that could be detected in the form of fluorescence signals. The assessment of the inhibitory effect of small molecules (EC₅₀) on IL4I1 is measured by the effectiveness of the compounds to inhibit the production of H₂O₂.

Using this assay, the potency (EC₅₀) of each compound was determined from a ten-point (1:3 serial dilution) titration curve using the following outlined procedure. To each well of a black flat-bottom Greiner (Cat #781076) 384 well-plate, 25 nL of compound (0.1% DMSO in final assay volume of 25 μL) was dispensed, followed by the addition of 12.5 μL of 1× assay buffer (50 mM Hepes 7.0 and 0.005% Tween20 (Sigma, Cat #P8341; low peroxide grade)) containing 2 nM of recombinant IL4I1 (R&D Systems, Cat #5684-AO-020). Plates were placed in an ambient temperature humidified chamber for a four-hour pre-incubation with compound. Subsequently, each reaction was initiated by the addition of 12.5 μL 1× assay buffer containing 2 mM of each aromatic amino acids (Phe/Tyr/Trp), 0.1 mM Amplex Red and 2 U/mL of HRP. The final reaction in each well of 25 μL consisted of 1 nM of IL4I1, 1 mM of each residues (Phe, Tyr and Trp), 0.05 mM Amplex Red and 1 U/mL of HRP. It should be noted that the concentrations of Amplex Red and HRP used here were in excess such that the conversion of H₂O₂ to resorufin product occurred instantaneously and was non-rate limiting. Reactions were allowed to proceed for 120 minutes followed by fluorescence readout on a Spectramax with the following set parameters: 544 nm excitation/590 nm emission, 570 nm cutoff (EnVision is an alternative reader). Dose-response curves were generated by plotting percent effect (% product conversion; Y-axis) vs. Log₁₀ compound concentrations (X-axis). EC₅₀ values were calculated using a non-linear regression, four-parameters sigmoidal dose-response model and are shown in Potency Table 1.

Potency Table 1

| Example | EC50 (nM) (240 min) |
|---|---|
| Example 1 | 10 |
| Example 2 | 9 |
| Example 3 | 12 |
| Example 4 | 19 |
| Example 5 | 50 |
| Example 6 | 43 |
| Example 7 | 9 |
| Example 8 | 7 |
| Example 9 | 15 |
| Example 10 | 21 |
| Example 11 | 16 |
| Example 12 | 8 |
| Example 13 | 8 |
| Example 14 | 6 |
| Example 15 | 44 |
| Example 16 | 16 |
| Example 17 | 7 |
| Example 18 | 41 |
| Example 19 | 7 |
| Example 20 | 5 |
| Example 21 | 14 |
| Example 22 | 22 |
| Example 23 | 21 |
| Example 24 | 30 |
| Example 25 | 21 |
| Example 26 | 6 |
| Example 27 | 5 |
| Example 28 | 8 |
| Example 29 | 9 |
| Example 30 | 8 |
| Example 31 | 6 |
| Example 32 | 5 |
| Example 33 | 8 |
| Example 34 | 34 |
| Example 35 | 3 |
| Example 36 | 6 |
| Example 37 | 18 |
| Example 38 | 23 |
| Example 39 | 3 |
| Example 40 | 4 |
| Example 41 | 2 |
| Example 42 | 5 |
| Example 43 | 3 |
| Example 44 | 7 |
| Example 45 | 5 |
| Example 46 | 13 |
| Example 47 | 3 |
| Example 48 | 2 |
| Example 49 | 3 |
| Example 50 | 4 |
| Example 51 | 8 |
| Example 52 | 4 |
| Example 53 | 7 |
| Example 54 | 2 |
| Example 55 | 1 |
| Example 56 | 1 |
| Example 57 | 3 |
| Example 58 | 4 |
| Example 59 | 5 |
| Example 60 | 10 |
| Example 61 | 5 |
| Example 62 | 4 |
| Example 63 | 3 |
| Example 64 | 4 |
| Example 65 | 4 |
| Example 66 | 7 |
| Example 67 | 1 |
| Example 68 | 5 |
| Example 69 | 3 |
| Example 70 | 4 |
| Example 71 | 6 |
| Example 72 | 2 |
| Example 73 | 9 |
| Example 74 | 5 |
| Example 75 | 7 |
| Example 76 | 4 |
| Example 77 | 3 |
| Example 78 | 6 |
| Example 79 | 5 |
| Example 80 | 15 |
| Example 81 | 60 |
| Example 82 | 10 |
| Example 83 | 8 |
| Example 84 | 13 |
| Example 85 | 29 |
| Example 86 | 27 |
| Example 87 | 87 |
| Example 88 | 69 |
| Example 89 | 32 |
| Example 90 | 63 |
| Example 91 | 162 |
| Example 92 | 155 |
| Example 93 | 31 |
| Example 94 | 35 |
| Example 95 | 16 |
| Example 96A | 14 |
| Example 96B | 8 |
| Example 97 | 5 |
| Example 98 | 12 |
| Example 99 | 12 |
| Example 100 | 8 |
| Example 101 | 8 |
| Example 102 | 14 |
| Example 103 | 11 |
| Example 104 | 4 |
| Example 105 | 2 |
| Example 106 | 7 |
| Example 107 | 13 |
| Example 108 | 20 |
| Example 109 | 24 |
| Example 110 | 27 |
| Example 111 | 8 |
| Example 112 | 7 |
| Example 113 | 15 |
| Example 114 | 7 |
| Example 115 | 4 |
| Example 116 | 17 |
| Example 117 | 14 |
| Example 118 | 7 |
| Example 119 | 4 |
| Example 120 | 3 |
| Example 121 | 7 |
| Example 122 | 6 |
| Example 123 | 5 |
| Example 124 | 7 |
| Example 125 | 3 |
| Example 126 | 3 |
| Example 127 | 3 |
| Example 128 | 2 |
| Example 129 | 6 |
| Example 130 | 5 |
| Example 131 | 9 |
| Example 132 | 15 |
| Example 133 | 22 |
| Example 134 | 40 |
| Example 135 | 6 |
| Example 136 | 20 |
| Example 137 | 6 |
| Example 138 | 12 |
| Example 139 | 26 |
| Example 140 | 36 |
| Example 141 | 23 |
| Example 142 | 3 |
| Example 143 | 15 |
| Example 144 | 2 |
| Example 145 | 1 |
| Example 146 | 3 |
| Example 147 | 2 |
| Example 148 | 2 |
| Example 149 | 3 |
| Example 150 | 3 |
| Example 151 | 1 |
| Example 152 | 2 |
| Example 153 | 3 |
| Example 154 | 3 |
| Example 155 | 2 |
| Example 156 | 2 |
| Example 157 | 1 |
| Example 158 | 4 |
| Example 159 | 1 |
| Example 160 | 5 |
| Example 161 | 5 |
| Example 162 | 4 |
| Example 163 | 6 |
| Example 164 | 1 |
| Example 165 | 12 |
| Example 166 | 10 |
| Example 167 | 9 |
| Example 168 | 6 |
| Example 169 | 7 |
| Example 170 | 4 |
| Example 171 | 6 |
| Example 172 | 7 |
| Example 173 | 14 |
| Example 174 | 39 |
| Example 175 | 14 |
| Example 176 | 40 |
| Example 177 | 84 |
| Example 178 | 52 |
| Example 179 | 13 |
| Example 180 | 97 |
| Example 181 | 10 |
| Example 182 | 14 |
| Example 183 | 24 |
| Example 184 | 25 |
| Example 185 | 8 |
| Example 186 | 4 |
| Example 187 | 8 |
| Example 188 | 10 |
| Example 189 | 4 |
| Example 190 | 31 |
| Example 191 | 35 |
| Example 192 | 13 |
| Example 193 | 8 |
| Example 194 | 41 |
| Example 195 | 48 |
| Example 196 | 11 |
| Example 197 | 33 |
| Example 198 | 76 |
| Example 199 | 24 |
| Example 200 | 68 |
| Example 201 | 30 |
| Example 202 | 44 |
| Example 203 | 19 |
| Example 204 | 78 |
| Example 205 | 24 |
| Example 206 | 28 |
| Example 207 | 43 |
| Example 208 | 21 |
| Example 209 | 26 |
| Example 210 | 92 |
| Example 211 | 61 |
| Example 212 | 10 |
| Example 213 | 29 |
| Example 214 | 56 |
| Example 215 | 57 |
| Example 216 | 60 |
| Example 217 | 42 |
| Example 218 | 56 |
| Example 219 | 59 |
| Example 220 | 50 |
| Example 221 | 14 |
| Example 222 | 316 |
| Example 223 | 10 |
| Example 224 | 101 |
| Example 225 | 41 |
| Example 226 | 78 |
| Example 227 | 69 |
| Example 228 | 23 |
| Example 229 | 54 |
| Example 230 | 91 |
| Example 231 | 22 |
| Example 232 | 82 |
| Example 233 | 54 |
| Example 234 | 40 |
| Example 235 | 18 |
| Example 236 | 533 |
| Example 237 | 346 |
| Example 238 | 29 |
| Example 239 | 82 |
| Example 240 | 995 |
| Example 241 | 40 |
| Example 242 | 32 |
| Example 243 | 30 |
| Example 244 | 45 |
| Example 245 | 43 |
| Example 246 | 37 |
| Example 247 | 44 |
| Example 248 | 38 |
| Example 249 | 148 |
| Example 250 | 240 |
| Example 251 | 100 |
| Example 252 | 38 |
| Example 253 | 45 |
| Example 254 | 93 |
| Example 255 | 47 |
| Example 256 | 533 |
| Example 257 | 68 |
| Example 258 | 78 |
| Example 259 | 78 |
| Example 260 | 38 |
| Example 261 | 178 |
| Example 262 | 868 |
| Example 263 | 995 |
| Example 264 | 73 |
| Example 265 | 114 |
| Example 266A | 4 |
| Example 266B | 4 |
| Example 267 | 57 |
| Example 268 | 15 |
| Example 269 | 49 |
| Example 270 | 24 |
| Example 271 | 14 |
| Example 272 | 35 |
| Example 273 | 19 |
| Example 274 | 34 |
| Example 275 | 20 |
| Example 276 | 26 |
| Example 277 | 24 |
| Example 278 | 16 |
| Example 279 | 68 |
| Example 280 | 38 |
| Example 281 | 35 |
| Example 282 | 30 |
| Example 283 | 7 |
| Example 284 | 8 |
| Example 285 | 28 |
| Example 286 | 34 |
| Example 287 | 34 |
| Example 288 | 37 |
| Example 289 | 6 |
| Example 290 | 8 |
| Example 291 | 4 |
| Example 292 | 64 |
| Example 293 | 11 |
| Example 294 | 16 |
| Example 295 | 53 |
| Example 296 | 63 |
| Example 297 | 83 |
| Example 298 | 32 |
| Example 299 | 56 |
| Example 300 | 86 |
| Example 301 | 67 |
| Example 302 | 69 |
| Example 303 | 28 |
| Example 304 | 98 |
| Example 305 | 32 |
| Example 306 | 27 |
| Example 307 | 20 |
| Example 308 | 35 |
| Example 309 | 41 |
| Example 310 | 41 |
| Example 311 | 60 |
| Example 312 | 69 |
| Example 313 | 47 |
| Example 314 | 43 |
| Example 315 | 23 |
| Example 316 | 32 |
| Example 317 | 45 |
| Example 318 | 36 |
| Example 319 | 30 |
| Example 320 | 20 |
| Example 321 | 21 |
| Example 322 | 28 |
| Example 323 | 157 |
| Example 324 | 53 |
| Example 325 | 33 |
| Example 326 | 20 |
| Example 327 | 16 |
| Example 328 | 7 |
| Example 329 | 15 |
| Example 330 | 16 |
| Example 331 | 9 |
| Example 332 | 76 |
| Example 333 | 94 |
| Example 334 | 29 |
| Example 335 | 90 |
| Example 336 | 20 |
| Example 337 | 15 |
| Example 338 | 12 |
| Example 339 | 42 |
| Example 340 | 70 |
| Example 341 | 19 |
| Example 342 | 69 |
| Example 343 | 133 |
| Example 344 | 49 |
| Example 345 | 59 |
| Example 346 | 7 |
| Example 347 | 74 |
| Example 348 | 18 |
| Example 349 | 25 |
| Example 350 | 5 |
| Example 351 | 8 |
| Example 352 | 13 |
| Example 353 | 11 |
| Example 354 | 20 |
| Example 355 | 22 |
| Example 356 | 28 |
| Example 357 | 32 |
| Example 358A | 2 |
| Example 358B | 1 |
| Example 359 | 22 |
| Example 360 | 16 |
| Example 361 | 96 |
| Example 362 | 22 |
| Example 363 | 25 |
| Example 364 | 17 |
| Example 365 | 17 |
| Example 366 | 9 |
| Example 367 | 9 |
| Example 368 | 28 |
| Example 369 | 32 |
| Example 370 | 20 |
| Example 371 | 42 |
| Example 372 | 23 |
| Example 373 | 8 |
| Example 374 | 8 |
| Example 375 | 6 |
| Example 376 | 14 |
| Example 377 | 71 |
| Example 378 | 138 |
| Example 379 | 24 |
| Example 380 | 17 |
| Example 381 | 40 |
| Example 382 | 25 |
| Example 383 | 26 |
| Example 384 | 9 |
| Example 385 | 42 |
| Example 386 | 225 |
| Example 387 | 677 |
| Example 388 | 23 |
| Example 389 | 369 |
| Example 390 | 343 |
| Example 391 | 170 |
| Example 392 | 31 |
| Example 393 | 33 |
| Example 394 | 31 |
| Example 395 | 8 |
| Example 396 | 5 |
| Example 397 | 11 |
| Example 398 | 22 |
| Example 399 | 19 |
| Example 400 | 25 |
| Example 401 | 48 |
| Example 402 | 36 |
| Example 403 | 17 |
| Example 404 | 10 |
| Example 405 | 23 |
| Example 406 | 15 |
| Example 407 | 27 |
| Example 408 | 8 |
| Example 409 | 9 |
| Example 410 | 5 |
| Example 411 | 7 |
| Example 412 | 19 |
| Example 413 | 66 |
| Example 414 | 15 |
| Example 415 | 138 |
| Example 416 | 28 |
| Example 417 | 2 |
| Example 418 | 6 |
| Example 419 | 2 |
| Example 420 | 5 |
| Example 421 | 5 |
| Example 422 | 5 |
| Example 423 | 19 |
| Example 424 | 19 |
| Example 425 | 8 |
| Example 426 | 5 |
| Example 427 | 19 |
| Example 428 | 21 |
| Example 429 | 29 |
| Example 430 | 183 |
| Example 431 | 130 |
| Example 432 | 17 |
| Example 433 | 37 |
| Example 434 | 71 |
| Example 435A | 85 |
| Example 435B | 76 |
| Example 436 | 20 |
| Example 437 | 34 |
| Example 438 | 40 |
| Example 439 | 9 |
| Example 440A | 5 |
| Example 440B | 6 |
| Example 440C | 10 |
| Example 440D | 8 |

1. A compound having a structural Formula I: 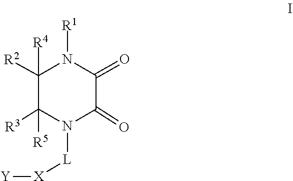
or a pharmaceutically acceptable salt thereof, wherein,
R¹ is C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl, wherein the C₁-C₆alkyl, C₃-C₆cycloalkyl, C₁-C₆alkylC₃-C₆cycloalkyl, aryl, heteroaryl, C₁-C₆alkylOC₁-C₆alkyl or heterocycloalkyl is unsubstituted or substituted with one to three substituents independently selected from the group consisting of oxo, —CN, —OH, halogen, C₁-C₆alkyl, C₁-C₆haloalkyl, phenyl, heteroaryl and C₃-C₆cycloalkyl, wherein the heteroaryl is unsubstituted or substituted with C₁-C₆alkoxy, or R¹, and the nitrogen to which it is attached, is taken with R⁴ and forms a nitrogen-containing ring;
R² is H, C₁-C₆alkyl or together with R³ forms a bond;
R³ is H, C₁-C₆alkyl or together with R² forms a bond;
R⁴ is H, halogen, or C₁-C₆alkyl or R⁴ is taken with R¹, and the nitrogen to which R¹ is attached, and forms a nitrogen-containing ring, or R⁴ is taken with R⁵ and forms a C₃-C₆cycloalkyl;
R⁵ is H, halogen, or C₁-C₆alkyl, or R⁵ is taken with R⁴ and forms a C₃-C₆cycloalkyl;
L is a C₁-C₆alkylene or C₃-C₆cycloalkylene linker;
X is phenyl, C₃-C₆cycloalkyl, or a nitrogen-containing ring, wherein the phenyl, C₃-C₆cycloalkyl or nitrogen-containing ring is unsubstituted or substituted with one or two substituents independently selected from the group consisting of halogen, —OH and oxo; and
Y is hydrogen, halogen, C₁-C₆alkoxy, phenyl, halogen-substituted phenyl, C₁-C₆alkyl-substituted phenyl, nitrogen-containing ring, CN-substituted phenyl, a halogen-substituted nitrogen-containing ring, or a haloC₁-C₆alkyl-substituted nitrogen-containing ring.

2. (canceled)

3. (canceled)

4. The compound of claim 1, or a pharmaceutically acceptable salt thereof, wherein R¹ is C₃-C₆cycloalkyl.

5. The compound of claim 1, or a pharmaceutically acceptable salt thereof, wherein R¹ is 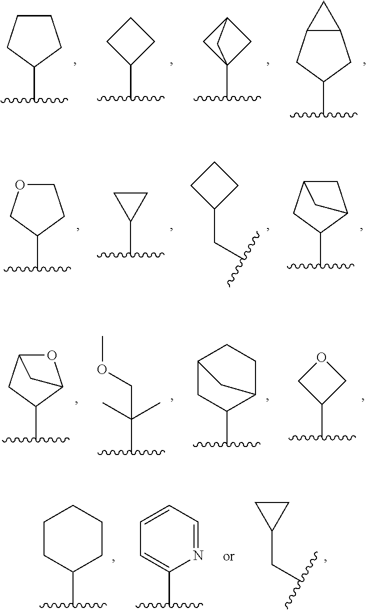
wherein R¹ is unsubstituted or substituted with one or two substituents independently selected from the group consisting of oxo, —CN, —OH, fluorine, methyl, phenyl, fluoromethyl, trifluoromethyl, pyridinyl, methoxypyridinyl and cyclopropyl.

6. The compound of claim 1, or a pharmaceutically acceptable salt thereof, wherein R² and R³ are both hydrogen.

7. (canceled)

8. (canceled)

9. (canceled)

10. The compound of claim 1, or a pharmaceutically acceptable salt thereof, wherein R⁴ and R⁵ are both hydrogen.

11. The compound of claim 1, or a pharmaceutically acceptable salt thereof, wherein L is

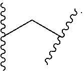 [SMILES: *C*]

12. The compound of claim 1, or a pharmaceutically acceptable salt thereof, wherein L is

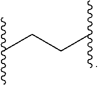 [SMILES: *CC*]

13. (canceled)

14. (canceled)

15. The compound of claim 1, or a pharmaceutically acceptable salt thereof, wherein X is phenyl, wherein the phenyl is unsubstituted or substituted one or two substitutents substituents independently selected from the group consisting of fluorine or bromine.

16. The compound of claim 1, or a pharmaceutically acceptable salt thereof, wherein X is a nitrogen-containing ring, wherein the nitrogen-containing ring is selected from the group consisting of 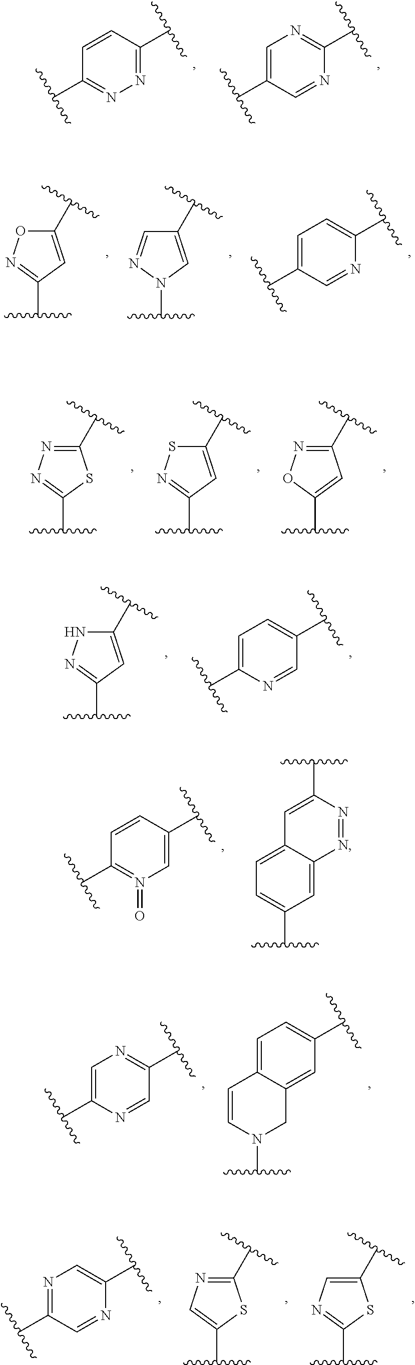 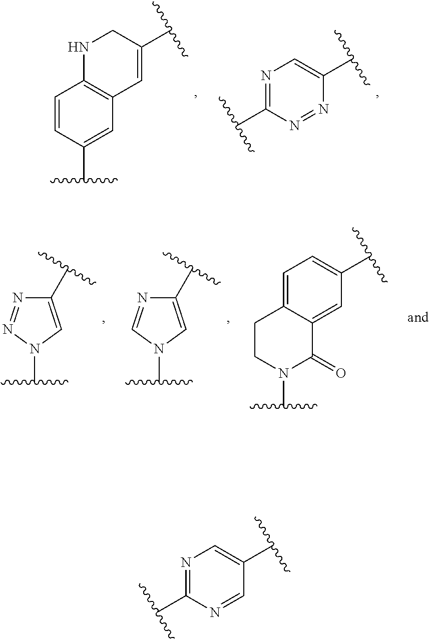
wherein X is unsubstituted or substituted one or two substituents independently selected from the group consisting of fluorine and an oxo group.

17. The compound of claim 1, or a pharmaceutically acceptable salt thereof, wherein X is a nitrogen-containing ring, wherein the nitrogen containing ring is 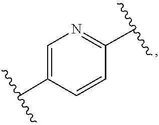
wherein X is substituted with a fluorine.

18. The compound of claim 1, or a pharmaceutically acceptable salt thereof, wherein X is a C₃-C₆cycloalkyl, wherein the C₃-C₆cycloalkyl is 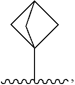
wherein X is unsubstituted or substituted with one or two fluorines.

19. The compound of claim 1, or a pharmaceutically acceptable salt thereof, wherein Y is phenyl.

20. The compound of claim 19, or a pharmaceutically acceptable salt thereof, wherein the phenyl is substituted with one to two fluorines.

21. (canceled)

22. The compound of claim 1, or a pharmaceutically acceptable salt thereof, wherein Y is a nitrogen-containing ring, wherein the nitrogen containing ring is selected from the group consisting of

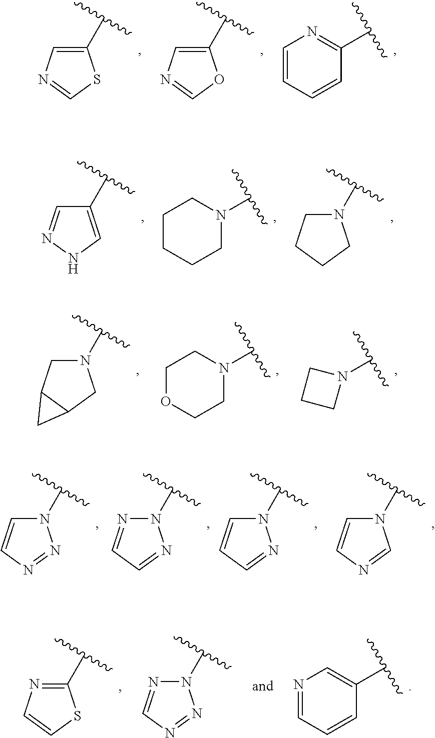

23. The compound of claim 1, or a pharmaceutically acceptable salt thereof, wherein Y is a halogen-substituted nitrogen-containing ring or a haloC₁-C₆alkyl-substituted nitrogen-containing ring, wherein the halogen-substituted nitrogen containing ring or haloC₁-C₆alkyl-substituted nitrogen-containing ring is selected from the group consisting of

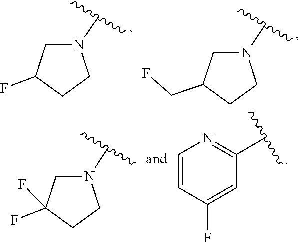

24. The compound of claim 1, or a pharmaceutically acceptable salt thereof, wherein Y is hydrogen only when X is

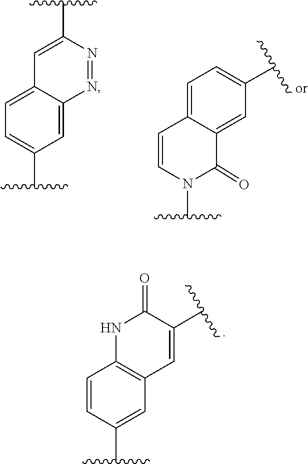 [SMILES: *c1ccc2cc(*)nnc2c1 ; *c1ccc2ccn(*)c(=O)c2c1 ; *c1ccc2[nH]c(=O)c(*)cc2c1]

25. (canceled)

26. (canceled)

27. The compound of claim 1, selected from the group consisting of 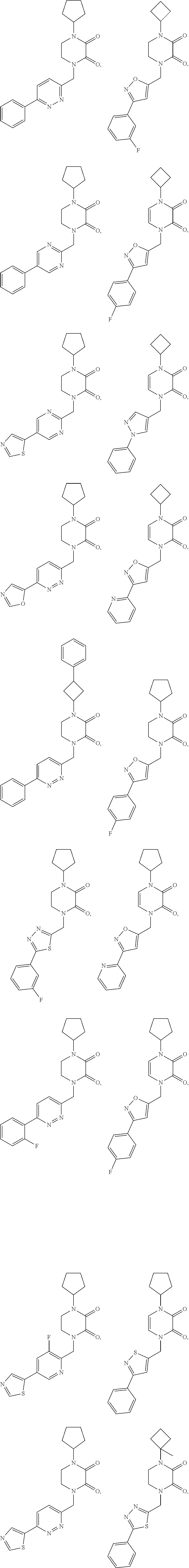 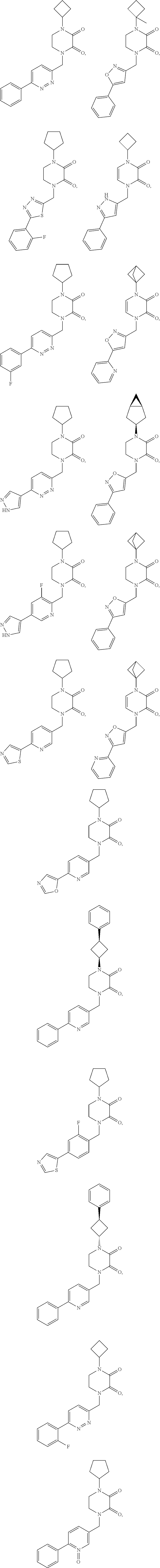 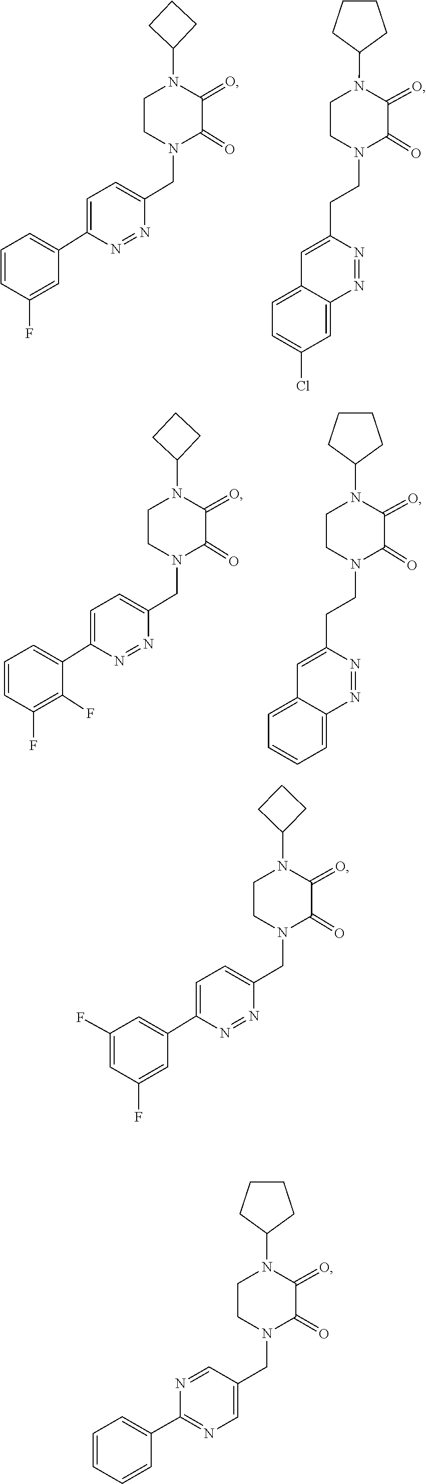 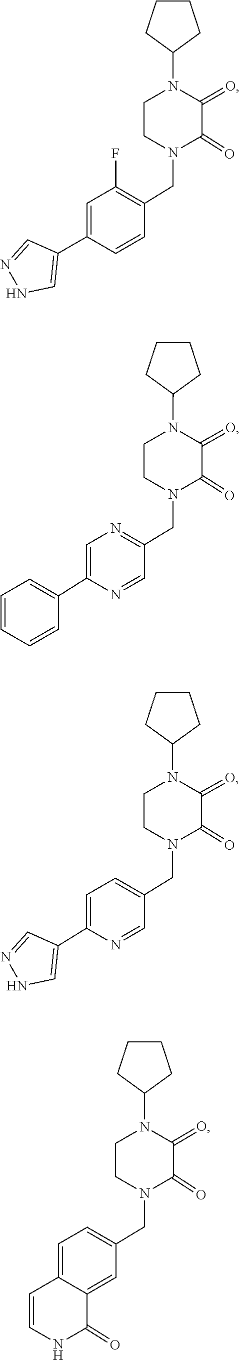 [SMILES: O=C1C(=O)N(C2CCCC2)CCN1Cc1ccc(-c2cn[nH]c2)cc1F ; O=C1C(=O)N(C2CCCC2)CCN1Cc1cnc(-c2ccccc2)cn1 ; O=C1C(=O)N(C2CCCC2)CCN1Cc1ccc(-c2cn[nH]c2)nc1 ; O=C1C(=O)N(C2CCCC2)CCN1Cc1ccc2cc[nH]c(=O)c2c1] 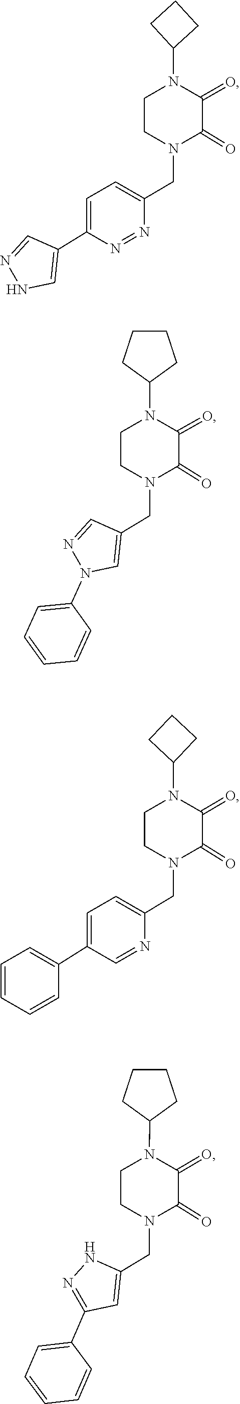 [SMILES: O=C1C(=O)N(C2CCC2)CCN1Cc1ccc(-c2cn[nH]c2)nn1 ; O=C1C(=O)N(C2CCCC2)CCN1Cc1cnn(-c2ccccc2)c1 ; O=C1C(=O)N(C2CCC2)CCN1Cc1ccc(-c2ccccc2)cn1 ; O=C1C(=O)N(C2CCCC2)CCN1Cc1cc(-c2ccccc2)n[nH]1] 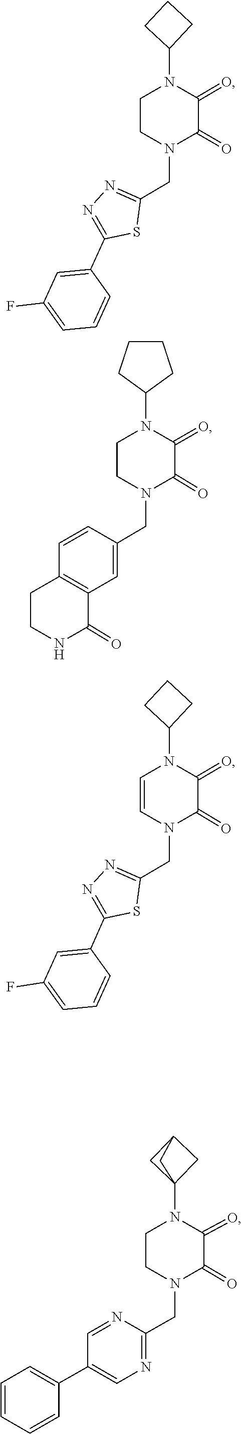 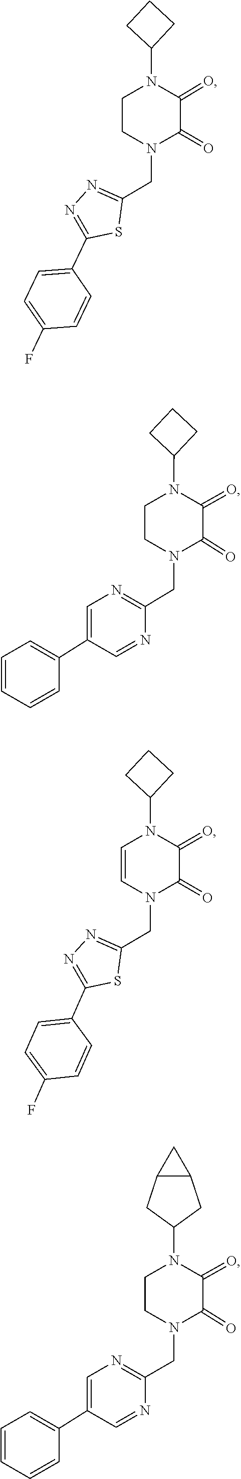 [SMILES: O=C1C(=O)N(C2CCC2)CCN1Cc1nnc(-c2ccc(F)cc2)s1 ; O=C1C(=O)N(C2CCC2)CCN1Cc1ncc(-c2ccccc2)cn1 ; O=c1c(=O)n(C2CCC2)ccn1Cc1nnc(-c2ccc(F)cc2)s1 ; O=C1C(=O)N(C2CC3CC3C2)CCN1Cc1ncc(-c2ccccc2)cn1] 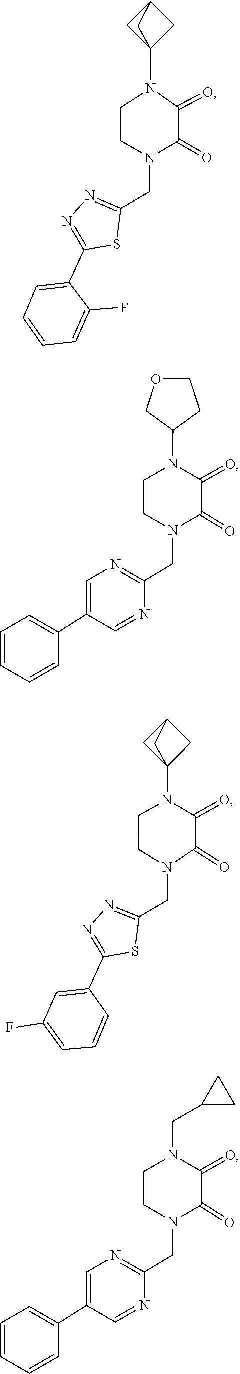 [SMILES: O=C1C(=O)N(C23CC(C2)C3)CCN1Cc1nnc(-c2ccccc2F)s1 ; O=C1C(=O)N(C2CCOC2)CCN1Cc1ncc(-c2ccccc2)cn1 ; O=C1C(=O)N(C23CC(C2)C3)CCN1Cc1nnc(-c2cccc(F)c2)s1 ; O=C1C(=O)N(CC2CC2)CCN1Cc1ncc(-c2ccccc2)cn1] 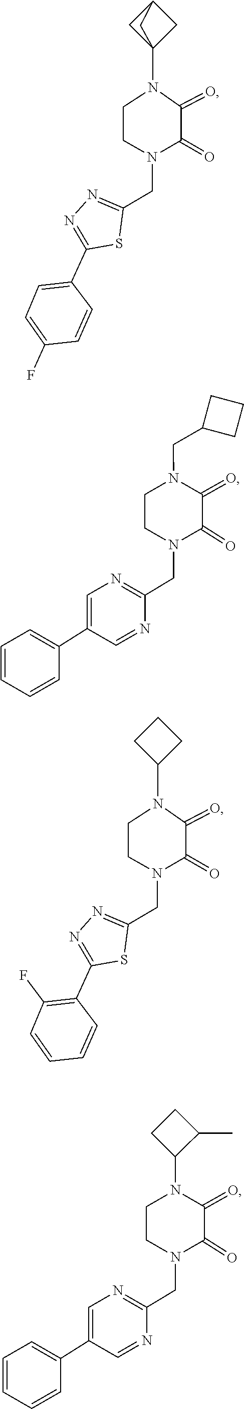 [SMILES: O=C1C(=O)N(C23CC(C2)C3)CCN1Cc1nnc(-c2ccc(F)cc2)s1 ; O=C1C(=O)N(CC2CCC2)CCN1Cc1ncc(-c2ccccc2)cn1 ; O=C1C(=O)N(C2CCC2)CCN1Cc1nnc(-c2ccccc2F)s1 ; CC1CCC1N1CCN(Cc2ncc(-c3ccccc3)cn2)C(=O)C1=O] 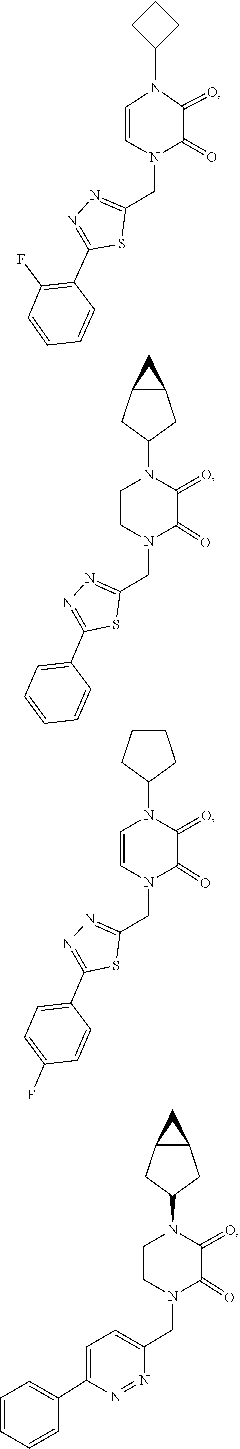 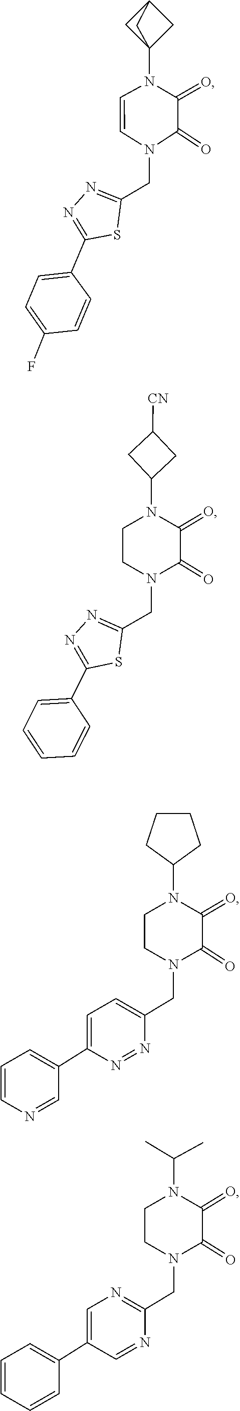 [SMILES: O=c1c(=O)n(C23CC(C2)C3)ccn1Cc1nnc(-c2ccc(F)cc2)s1 ; N#CC1CC(N2CCN(Cc3nnc(-c4ccccc4)s3)C(=O)C2=O)C1 ; O=C1C(=O)N(C2CCCC2)CCN1Cc1ccc(-c2cccnc2)nn1 ; CC(C)N1CCN(Cc2ncc(-c3ccccc3)cn2)C(=O)C1=O] 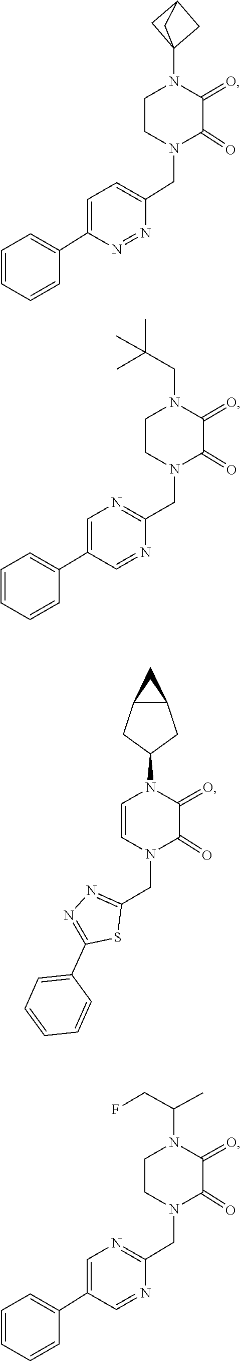 [SMILES: O=C1C(=O)N(C23CC(C2)C3)CCN1Cc1ccc(-c2ccccc2)nn1 ; CC(C)(C)CN1CCN(Cc2ncc(-c3ccccc3)cn2)C(=O)C1=O ; O=c1c(=O)n([C@H]2C[C@H]3C[C@H]3C2)ccn1Cc1nnc(-c2ccccc2)s1 ; CC(CF)N1CCN(Cc2ncc(-c3ccccc3)cn2)C(=O)C1=O] 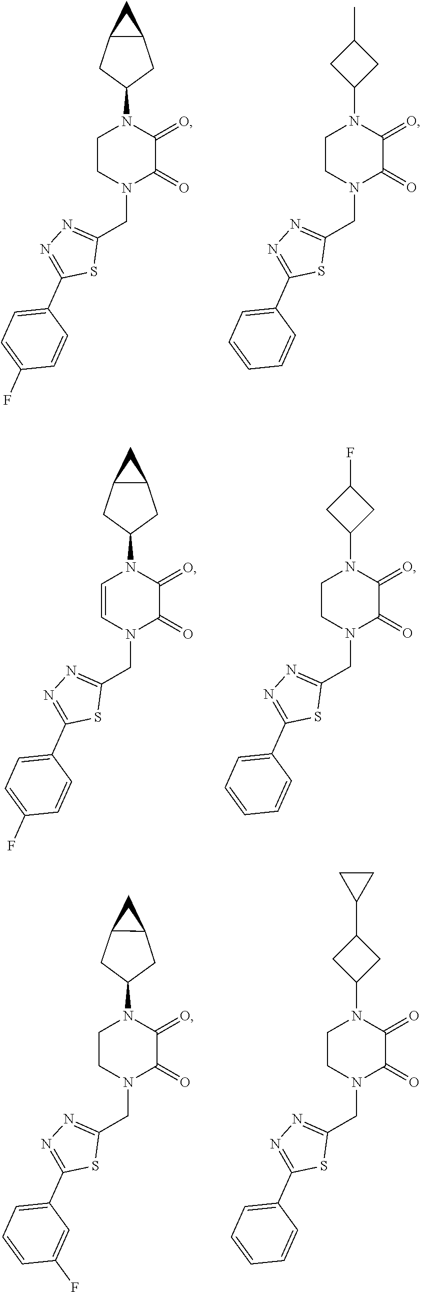 [SMILES: O=C1C(=O)N([C@H]2C[C@H]3C[C@H]3C2)CCN1Cc1nnc(-c2ccc(F)cc2)s1 ; CC1CC(N2CCN(Cc3nnc(-c4ccccc4)s3)C(=O)C2=O)C1 ; O=c1c(=O)n([C@H]2C[C@H]3C[C@H]3C2)ccn1Cc1nnc(-c2ccc(F)cc2)s1 ; O=C1C(=O)N(C2CC(F)C2)CCN1Cc1nnc(-c2ccccc2)s1 ; O=C1C(=O)N([C@H]2C[C@H]3C[C@H]3C2)CCN1Cc1nnc(-c2cccc(F)c2)s1 ; O=C1C(=O)N(C2CC(C3CC3)C2)CCN1Cc1nnc(-c2ccccc2)s1] 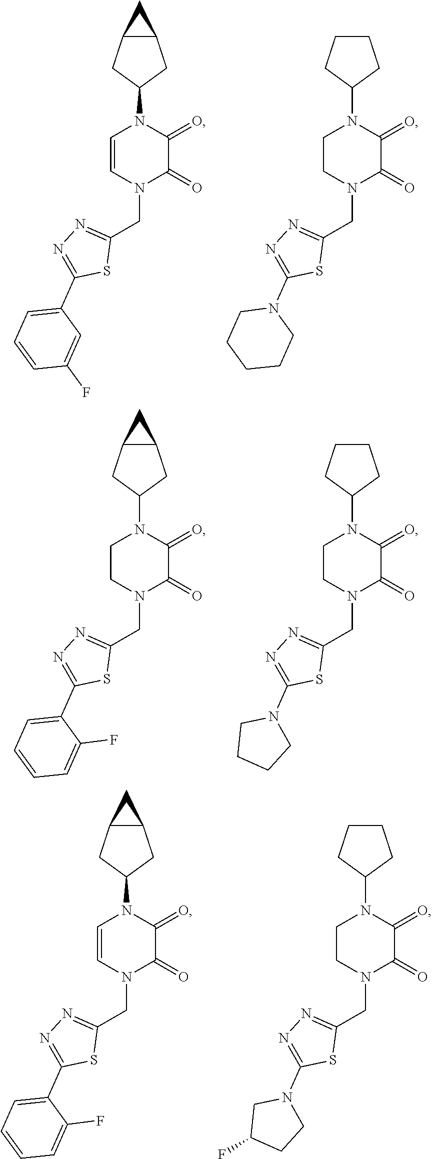 [SMILES: O=c1c(=O)n([C@H]2C[C@H]3C[C@H]3C2)ccn1Cc1nnc(-c2cccc(F)c2)s1 ; O=C1C(=O)N(C2CCCC2)CCN1Cc1nnc(N2CCCCC2)s1 ; O=C1C(=O)N(C2C[C@@H]3C[C@@H]3C2)CCN1Cc1nnc(-c2ccccc2F)s1 ; O=C1C(=O)N(C2CCCC2)CCN1Cc1nnc(N2CCCC2)s1 ; O=c1c(=O)n([C@H]2C[C@H]3C[C@H]3C2)ccn1Cc1nnc(-c2ccccc2F)s1 ; O=C1C(=O)N(C2CCCC2)CCN1Cc1nnc(N2CC[C@H](F)C2)s1] 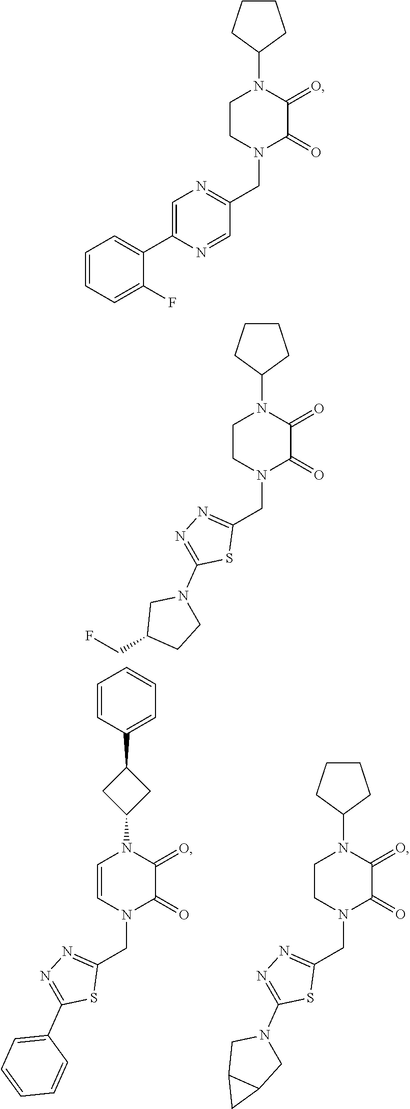 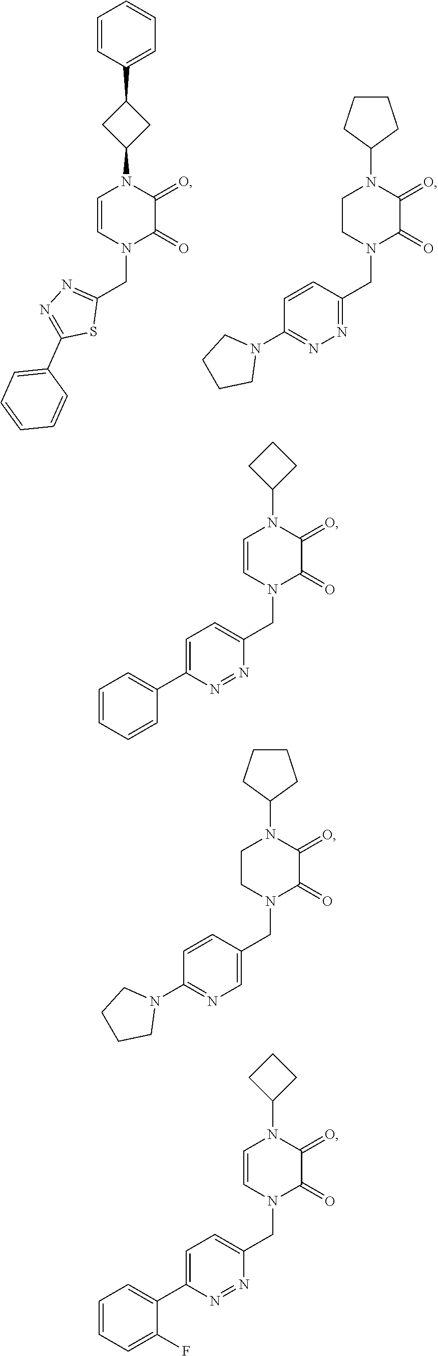 [SMILES: O=c1c(=O)n([C@H]2C[C@@H](c3ccccc3)C2)ccn1Cc1nnc(-c2ccccc2)s1 ; O=C1C(=O)N(C2CCCC2)CCN1Cc1ccc(N2CCCC2)nn1 ; O=c1c(=O)n(C2CCC2)ccn1Cc1ccc(-c2ccccc2)nn1 ; O=C1C(=O)N(C2CCCC2)CCN1Cc1ccc(N2CCCC2)nc1 ; O=c1c(=O)n(C2CCC2)ccn1Cc1ccc(-c2ccccc2F)nn1] 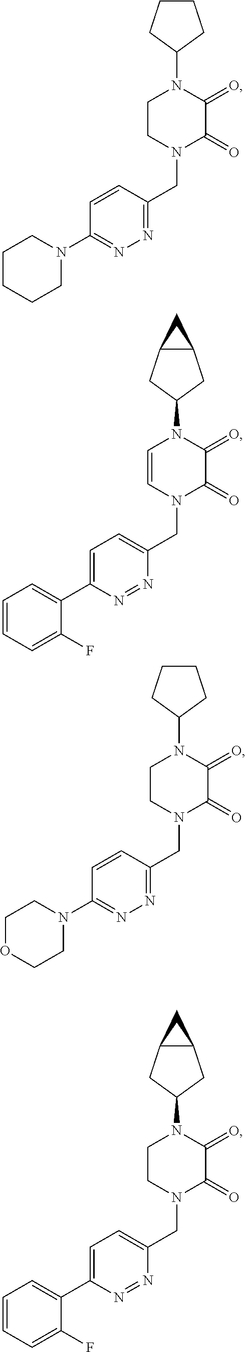 [SMILES: O=C1C(=O)N(C2CCCC2)CCN1Cc1ccc(N2CCCCC2)nn1 ; O=c1c(=O)n([C@H]2C[C@H]3C[C@H]3C2)ccn1Cc1ccc(-c2ccccc2F)nn1 ; O=C1C(=O)N(C2CCCC2)CCN1Cc1ccc(N2CCOCC2)nn1 ; O=C1C(=O)N([C@H]2C[C@H]3C[C@H]3C2)CCN1Cc1ccc(-c2ccccc2F)nn1] 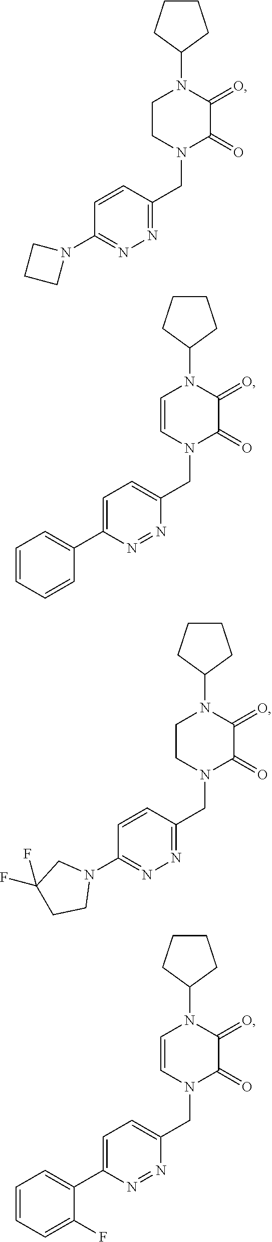 [SMILES: O=C1C(=O)N(C2CCCC2)CCN1Cc1ccc(N2CCC2)nn1 ; O=c1c(=O)n(C2CCCC2)ccn1Cc1ccc(-c2ccccc2)nn1 ; O=C1C(=O)N(C2CCCC2)CCN1Cc1ccc(N2CCC(F)(F)C2)nn1 ; O=c1c(=O)n(C2CCCC2)ccn1Cc1ccc(-c2ccccc2F)nn1] 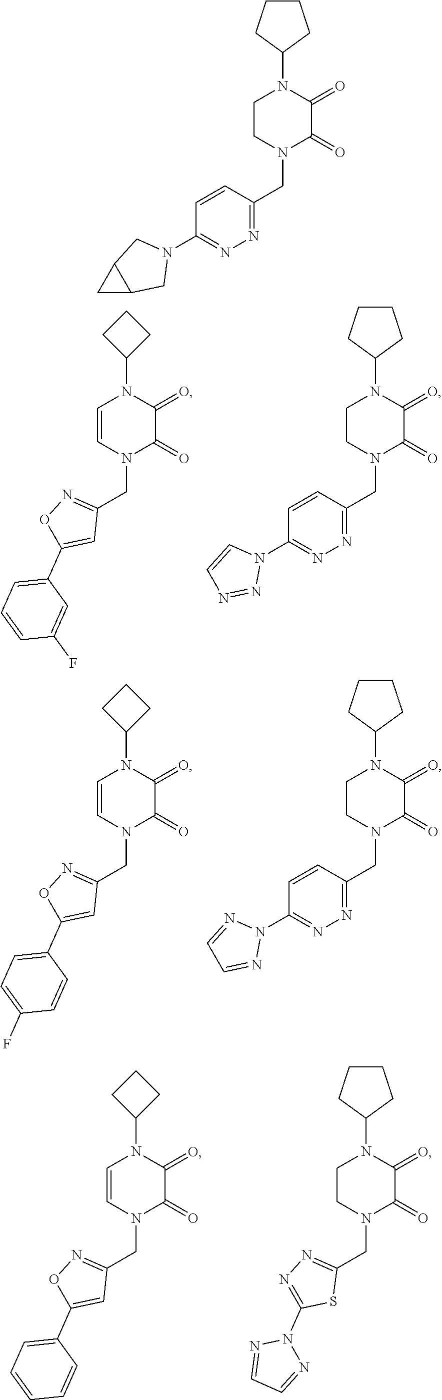 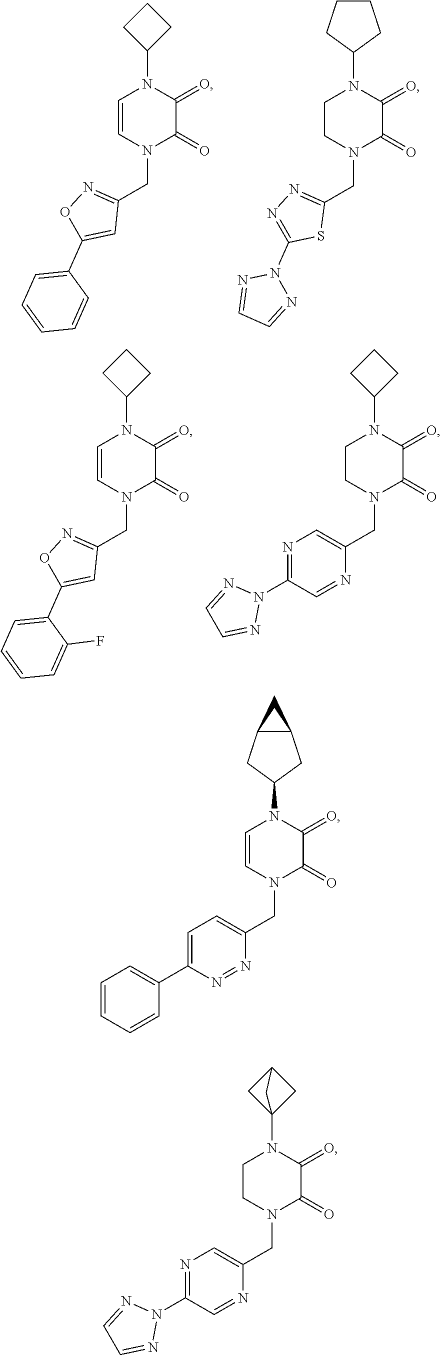 [SMILES: O=c1c(=O)n(C2CCC2)ccn1Cc1cc(-c2ccccc2)on1 ; O=C1C(=O)N(C2CCCC2)CCN1Cc1nnc(-n2nccn2)s1 ; O=c1c(=O)n(C2CCC2)ccn1Cc1cc(-c2ccccc2F)on1 ; O=C1C(=O)N(C2CCC2)CCN1Cc1cnc(-n2nccn2)cn1 ; O=c1c(=O)n([C@H]2C[C@H]3C[C@H]3C2)ccn1Cc1ccc(-c2ccccc2)nn1 ; O=C1C(=O)N(C23CC(C2)C3)CCN1Cc1cnc(-n2nccn2)cn1] 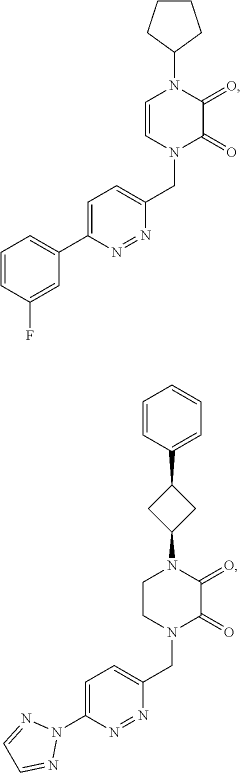 [SMILES: O=c1c(=O)n(C2CCCC2)ccn1Cc1ccc(-c2cccc(F)c2)nn1 ; O=C1C(=O)N([C@H]2C[C@@H](c3ccccc3)C2)CCN1Cc1ccc(-n2nccn2)nn1] 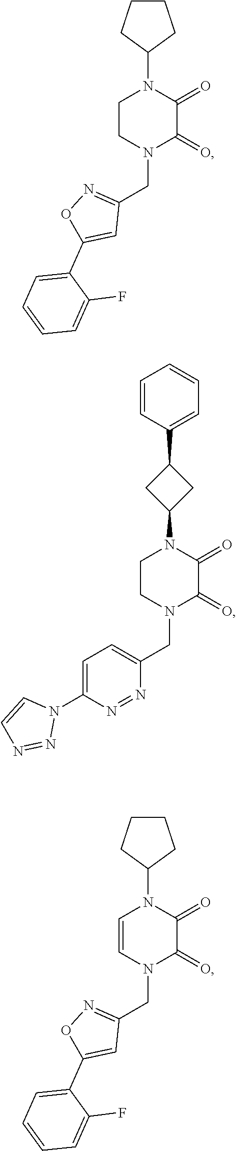 [SMILES: O=C1C(=O)N(C2CCCC2)CCN1Cc1cc(-c2ccccc2F)on1 ; O=C1C(=O)N([C@H]2C[C@@H](c3ccccc3)C2)CCN1Cc1ccc(-n2ccnn2)nn1 ; O=c1c(=O)n(C2CCCC2)ccn1Cc1cc(-c2ccccc2F)on1] 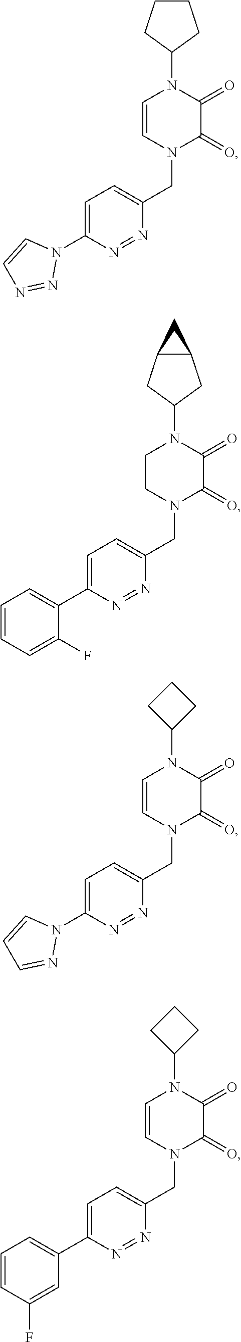 [SMILES: O=c1c(=O)n(C2CCCC2)ccn1Cc1ccc(-n2ccnn2)nn1 ; O=C1C(=O)N(C2C[C@@H]3C[C@@H]3C2)CCN1Cc1ccc(-c2ccccc2F)nn1 ; O=c1c(=O)n(C2CCC2)ccn1Cc1ccc(-n2cccn2)nn1 ; O=c1c(=O)n(C2CCC2)ccn1Cc1ccc(-c2cccc(F)c2)nn1] 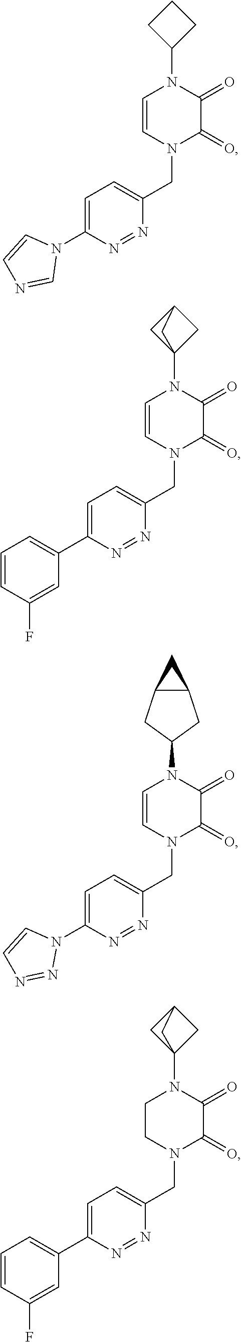 [SMILES: O=c1c(=O)n(C2CCC2)ccn1Cc1ccc(-n2ccnc2)nn1 ; O=c1c(=O)n(C23CC(C2)C3)ccn1Cc1ccc(-c2cccc(F)c2)nn1 ; O=c1c(=O)n([C@H]2C[C@H]3C[C@H]3C2)ccn1Cc1ccc(-n2ccnn2)nn1 ; O=C1C(=O)N(C23CC(C2)C3)CCN1Cc1ccc(-c2cccc(F)c2)nn1] 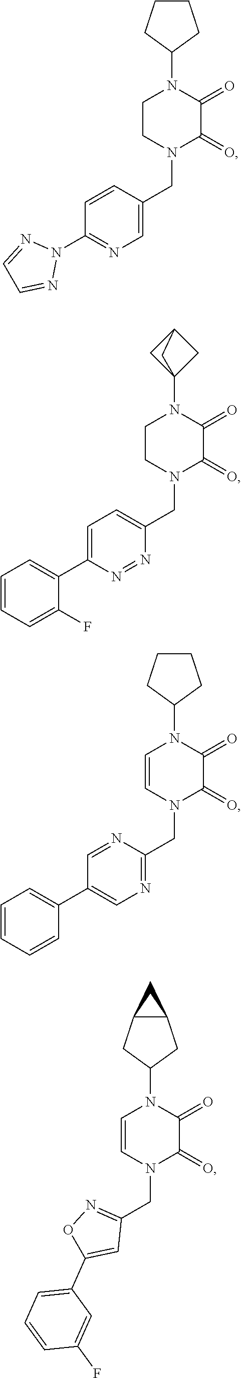 [SMILES: O=C1C(=O)N(C2CCCC2)CCN1Cc1ccc(-n2nccn2)nc1 ; O=C1C(=O)N(C23CC(C2)C3)CCN1Cc1ccc(-c2ccccc2F)nn1 ; O=c1c(=O)n(C2CCCC2)ccn1Cc1ncc(-c2ccccc2)cn1 ; O=c1c(=O)n(C2C[C@@H]3C[C@@H]3C2)ccn1Cc1cc(-c2cccc(F)c2)on1] 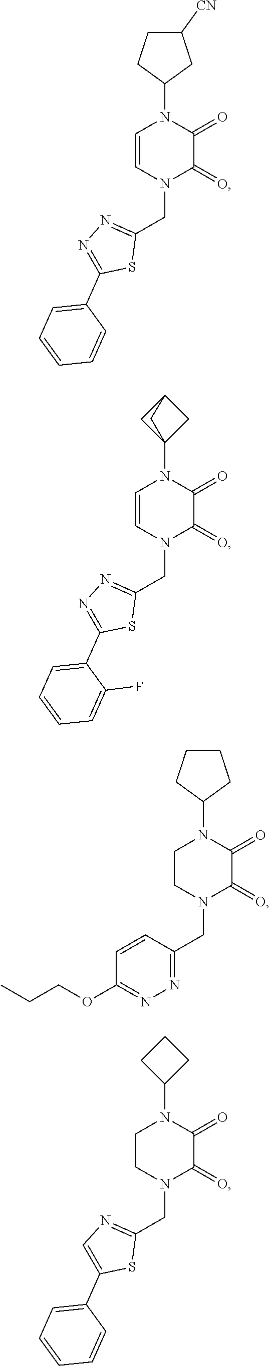 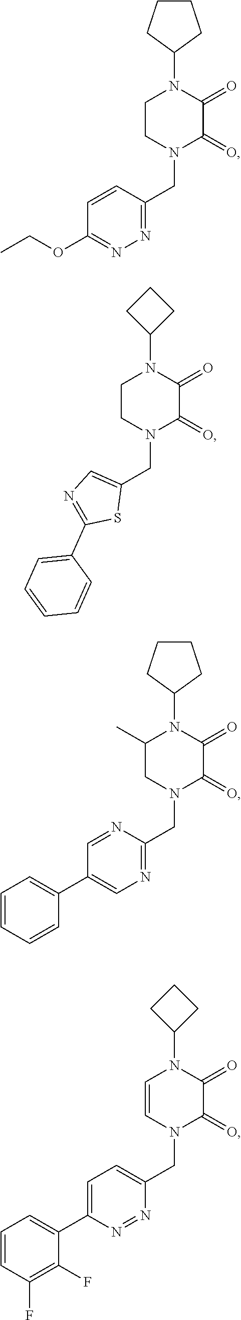 [SMILES: CCOc1ccc(CN2CCN(C3CCCC3)C(=O)C2=O)nn1 ; O=C1C(=O)N(C2CCC2)CCN1Cc1cnc(-c2ccccc2)s1 ; CC1CN(Cc2ncc(-c3ccccc3)cn2)C(=O)C(=O)N1C1CCCC1 ; O=c1c(=O)n(C2CCC2)ccn1Cc1ccc(-c2cccc(F)c2F)nn1] 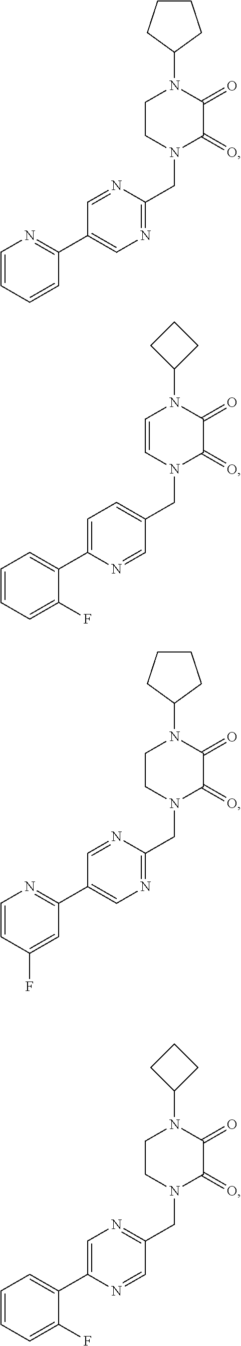 [SMILES: O=C1C(=O)N(C2CCCC2)CCN1Cc1ncc(-c2ccccn2)cn1 ; O=c1c(=O)n(C2CCC2)ccn1Cc1ccc(-c2ccccc2F)nc1 ; O=C1C(=O)N(C2CCCC2)CCN1Cc1ncc(-c2cc(F)ccn2)cn1 ; O=C1C(=O)N(C2CCC2)CCN1Cc1cnc(-c2ccccc2F)cn1] 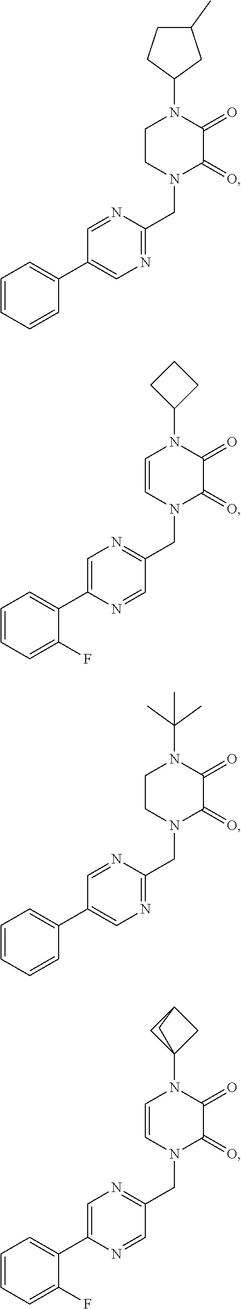 [SMILES: CC1CCC(N2CCN(Cc3ncc(-c4ccccc4)cn3)C(=O)C2=O)C1 ; O=c1c(=O)n(C2CCC2)ccn1Cc1cnc(-c2ccccc2F)cn1 ; CC(C)(C)N1CCN(Cc2ncc(-c3ccccc3)cn2)C(=O)C1=O ; O=c1c(=O)n(C23CC(C2)C3)ccn1Cc1cnc(-c2ccccc2F)cn1] 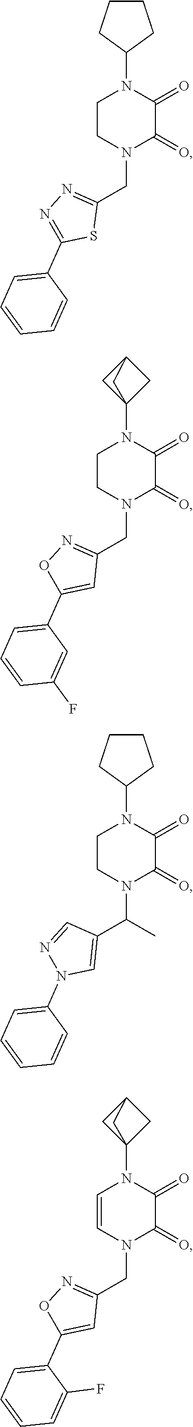 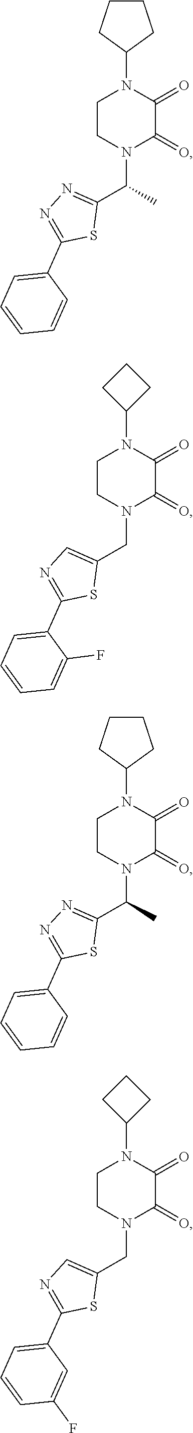 [SMILES: C[C@H](c1nnc(-c2ccccc2)s1)N1CCN(C2CCCC2)C(=O)C1=O ; O=C1C(=O)N(C2CCC2)CCN1Cc1cnc(-c2ccccc2F)s1 ; C[C@@H](c1nnc(-c2ccccc2)s1)N1CCN(C2CCCC2)C(=O)C1=O ; O=C1C(=O)N(C2CCC2)CCN1Cc1cnc(-c2cccc(F)c2)s1] 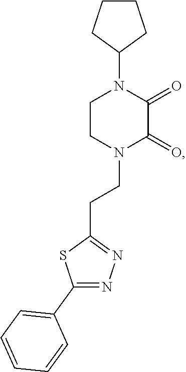 [SMILES: O=C1C(=O)N(C2CCCC2)CCN1CCc1nnc(-c2ccccc2)s1] 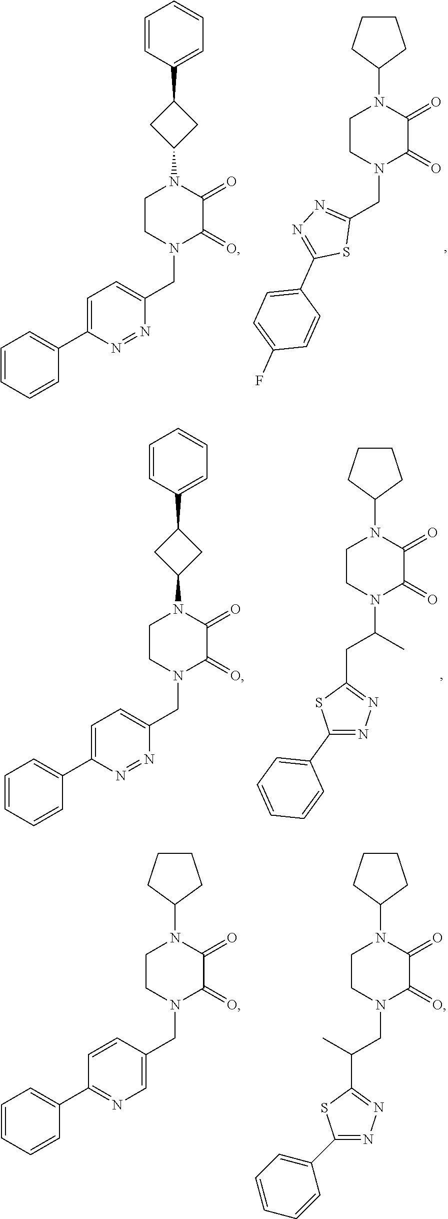 [SMILES: O=C1C(=O)N([C@H]2C[C@H](c3ccccc3)C2)CCN1Cc1ccc(-c2ccccc2)nn1 ; O=C1C(=O)N(C2CCCC2)CCN1Cc1nnc(-c2ccc(F)cc2)s1 ; O=C1C(=O)N([C@H]2C[C@@H](c3ccccc3)C2)CCN1Cc1ccc(-c2ccccc2)nn1 ; CC(Cc1nnc(-c2ccccc2)s1)N1CCN(C2CCCC2)C(=O)C1=O ; O=C1C(=O)N(C2CCCC2)CCN1Cc1ccc(-c2ccccc2)nc1 ; CC(CN1CCN(C2CCCC2)C(=O)C1=O)c1nnc(-c2ccccc2)s1] 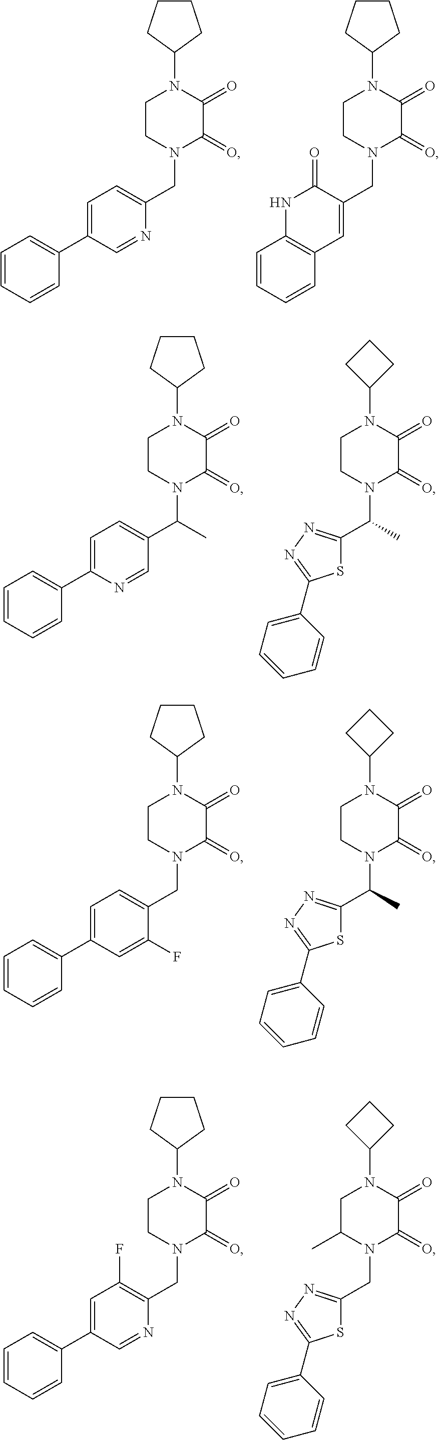 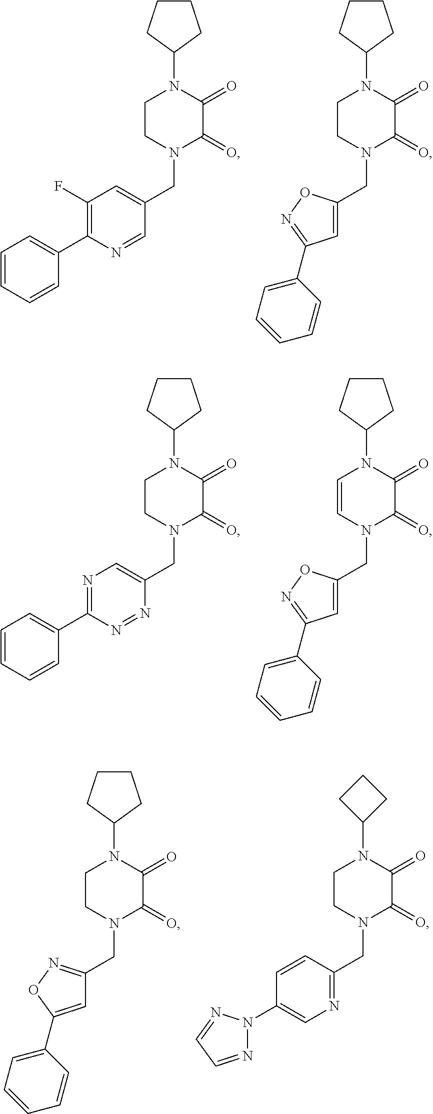 [SMILES: O=C1C(=O)N(C2CCCC2)CCN1Cc1cnc(-c2ccccc2)c(F)c1 ; O=C1C(=O)N(C2CCCC2)CCN1Cc1cc(-c2ccccc2)no1 ; O=C1C(=O)N(C2CCCC2)CCN1Cc1cnc(-c2ccccc2)nn1 ; O=c1c(=O)n(C2CCCC2)ccn1Cc1cc(-c2ccccc2)no1 ; O=C1C(=O)N(C2CCCC2)CCN1Cc1cc(-c2ccccc2)on1 ; O=C1C(=O)N(C2CCC2)CCN1Cc1ccc(-n2nccn2)cn1] 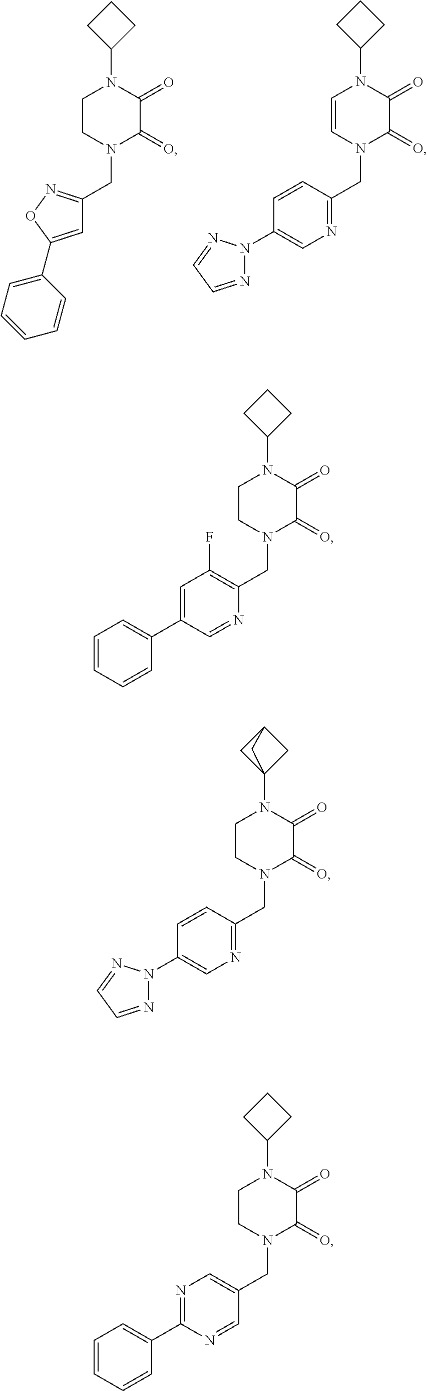 [SMILES: O=C1C(=O)N(C2CCC2)CCN1Cc1cc(-c2ccccc2)on1 ; O=c1c(=O)n(C2CCC2)ccn1Cc1ccc(-n2nccn2)cn1 ; O=C1C(=O)N(C2CCC2)CCN1Cc1ncc(-c2ccccc2)cc1F ; O=C1C(=O)N(C23CC(C2)C3)CCN1Cc1ccc(-n2nccn2)cn1 ; O=C1C(=O)N(C2CCC2)CCN1Cc1cnc(-c2ccccc2)nc1] 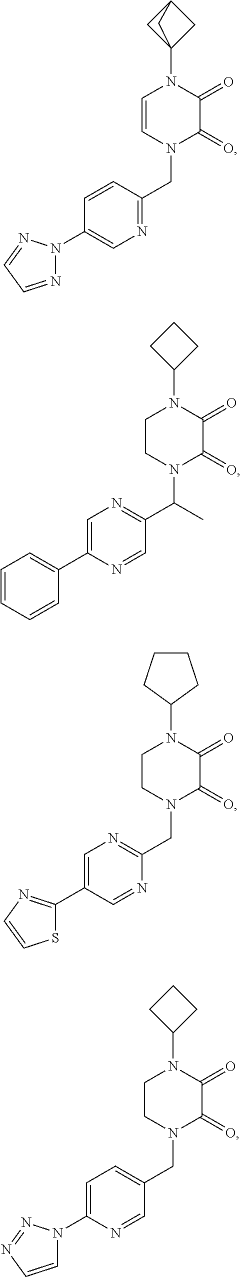 [SMILES: O=c1c(=O)n(C23CC(C2)C3)ccn1Cc1ccc(-n2nccn2)cn1 ; CC(c1cnc(-c2ccccc2)cn1)N1CCN(C2CCC2)C(=O)C1=O ; O=C1C(=O)N(C2CCCC2)CCN1Cc1ncc(-c2nccs2)cn1 ; O=C1C(=O)N(C2CCC2)CCN1Cc1ccc(-n2ccnn2)nc1] 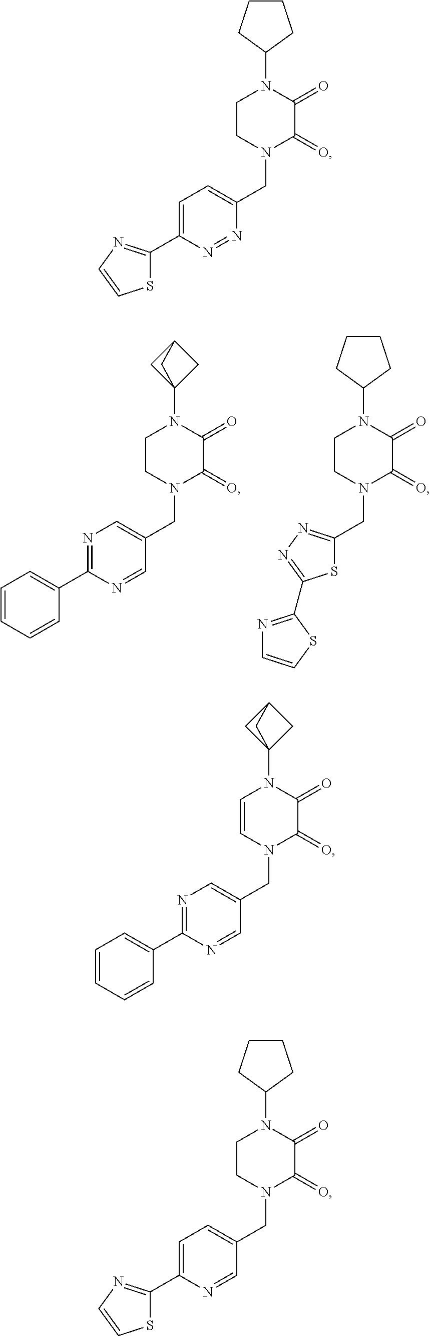 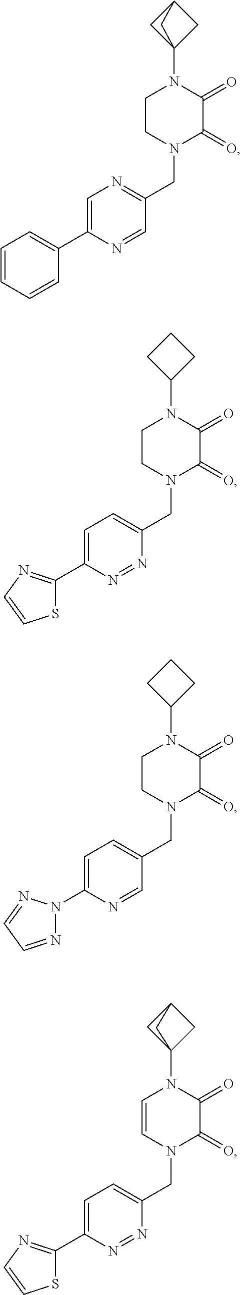 [SMILES: O=C1C(=O)N(C23CC(C2)C3)CCN1Cc1cnc(-c2ccccc2)cn1 ; O=C1C(=O)N(C2CCC2)CCN1Cc1ccc(-c2nccs2)nn1 ; O=C1C(=O)N(C2CCC2)CCN1Cc1ccc(-n2nccn2)nc1 ; O=c1c(=O)n(C23CC(C2)C3)ccn1Cc1ccc(-c2nccs2)nn1] 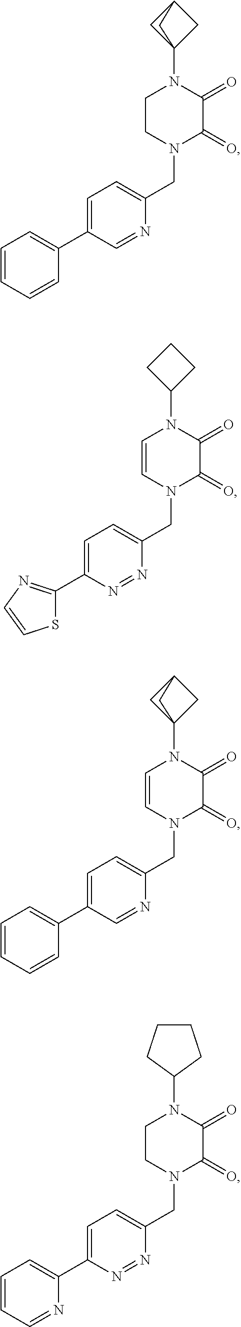 [SMILES: O=C1C(=O)N(C23CC(C2)C3)CCN1Cc1ccc(-c2ccccc2)cn1 ; O=c1c(=O)n(C2CCC2)ccn1Cc1ccc(-c2nccs2)nn1 ; O=c1c(=O)n(C23CC(C2)C3)ccn1Cc1ccc(-c2ccccc2)cn1 ; O=C1C(=O)N(C2CCCC2)CCN1Cc1ccc(-c2ccccn2)nn1] 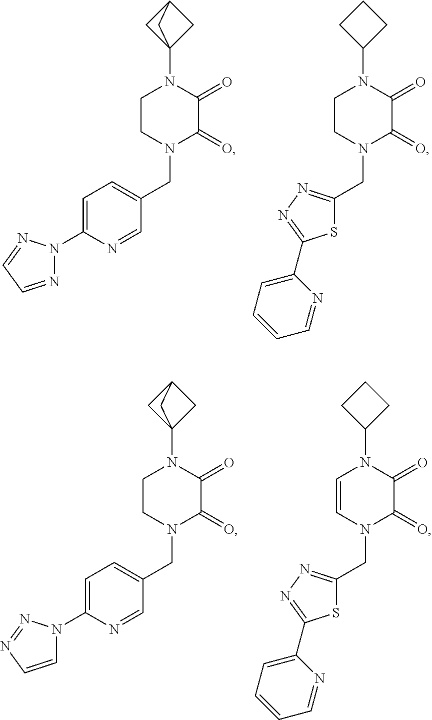 [SMILES: O=C1C(=O)N(C23CC(C2)C3)CCN1Cc1ccc(-n2nccn2)nc1 ; O=C1C(=O)N(C2CCC2)CCN1Cc1nnc(-c2ccccn2)s1 ; O=C1C(=O)N(C23CC(C2)C3)CCN1Cc1ccc(-n2ccnn2)nc1 ; O=c1c(=O)n(C2CCC2)ccn1Cc1nnc(-c2ccccn2)s1] 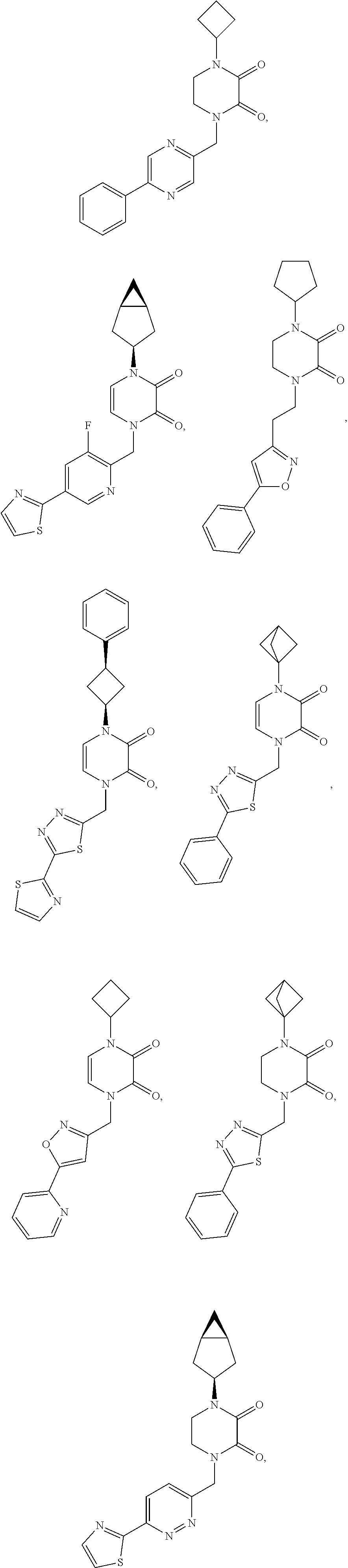 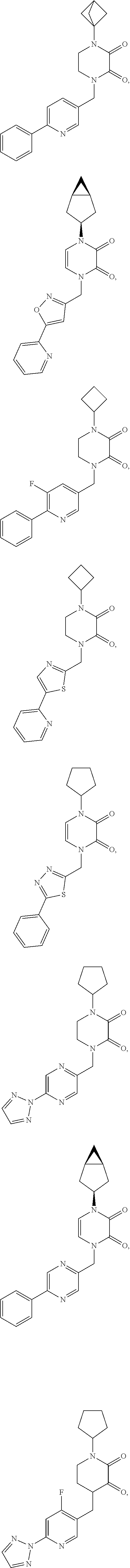 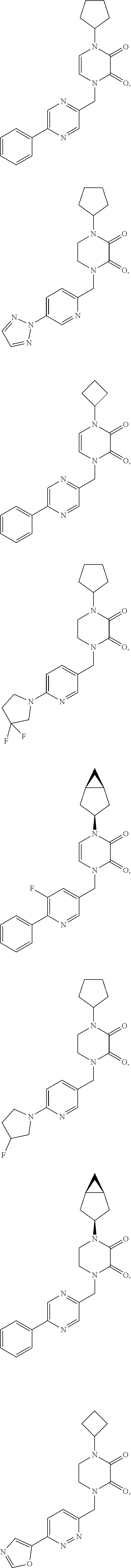 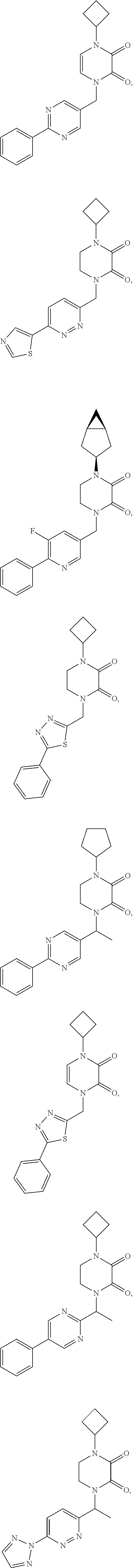 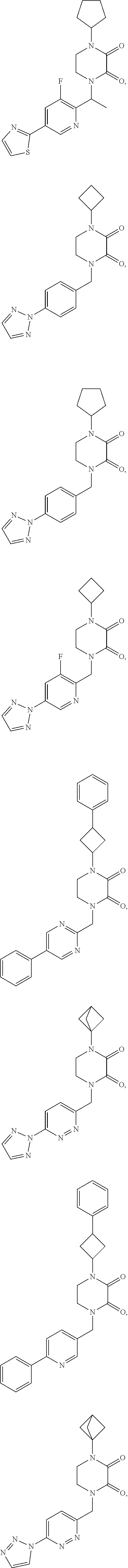 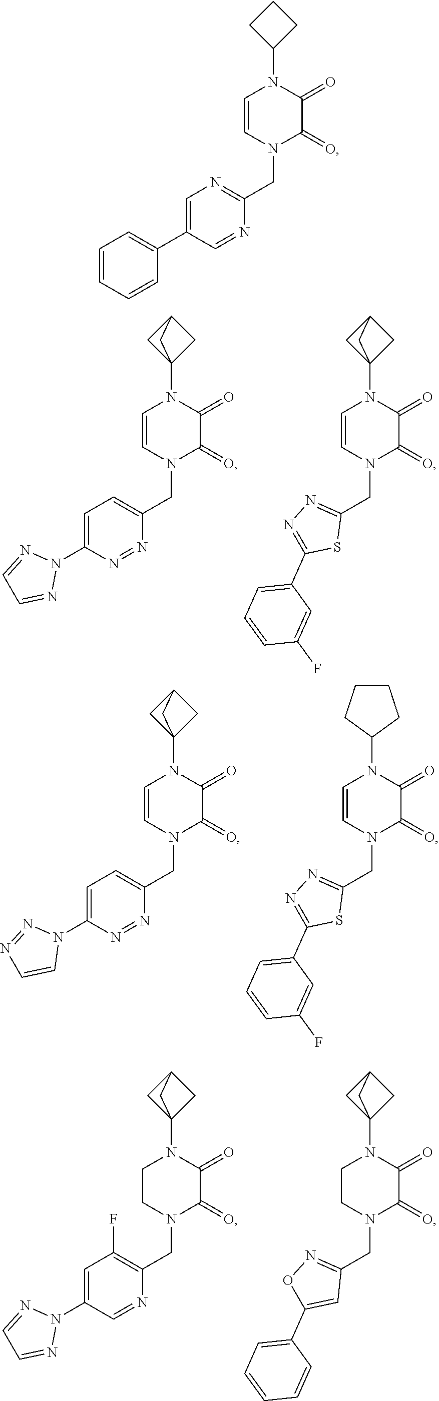 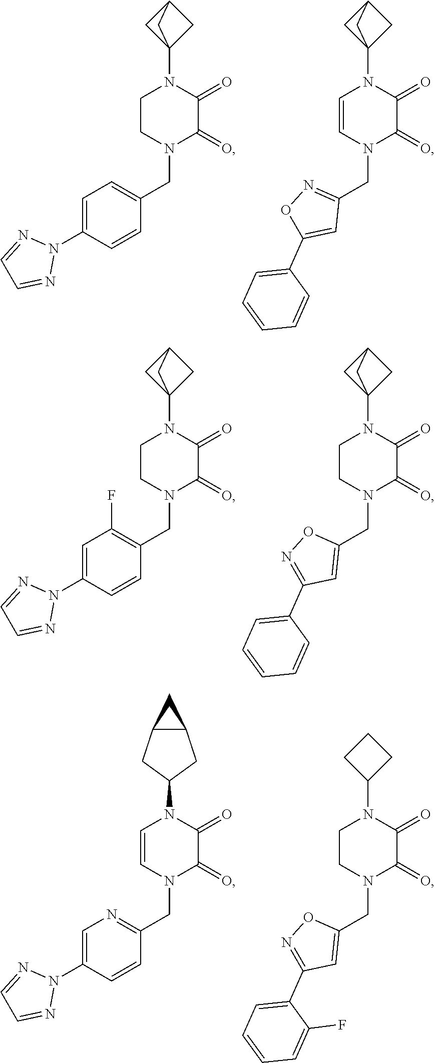 [SMILES: O=C1C(=O)N(C23CC(C2)C3)CCN1Cc1ccc(-n2nccn2)cc1 ; O=c1c(=O)n(C23CC(C2)C3)ccn1Cc1cc(-c2ccccc2)on1 ; O=C1C(=O)N(C23CC(C2)C3)CCN1Cc1ccc(-n2nccn2)cc1F ; O=C1C(=O)N(C23CC(C2)C3)CCN1Cc1cc(-c2ccccc2)no1 ; O=c1c(=O)n([C@H]2C[C@H]3C[C@H]3C2)ccn1Cc1ccc(-n2nccn2)cn1 ; O=C1C(=O)N(C2CCC2)CCN1Cc1cc(-c2ccccc2F)no1] 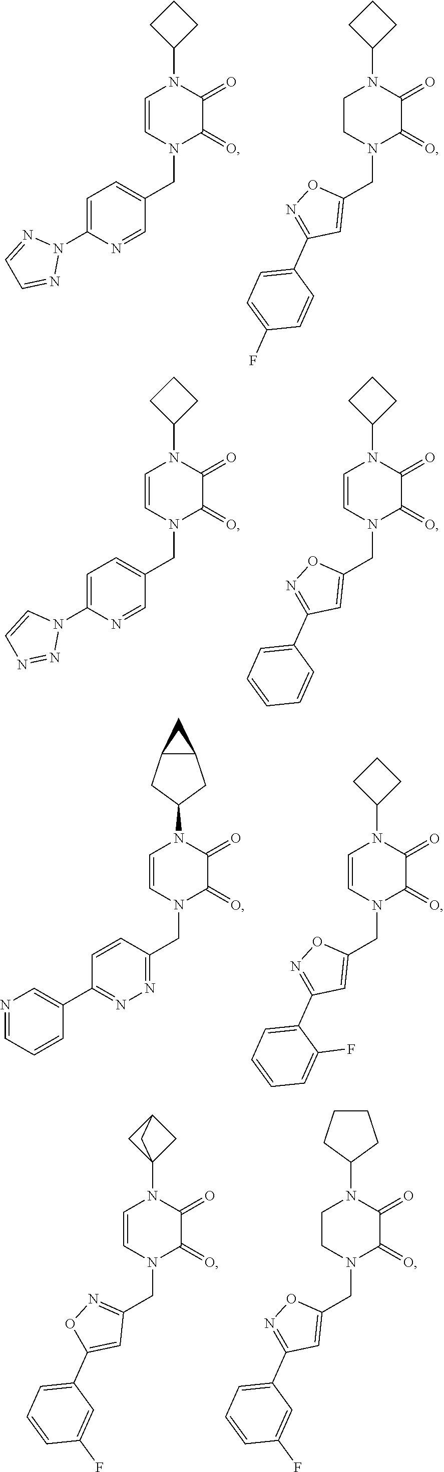 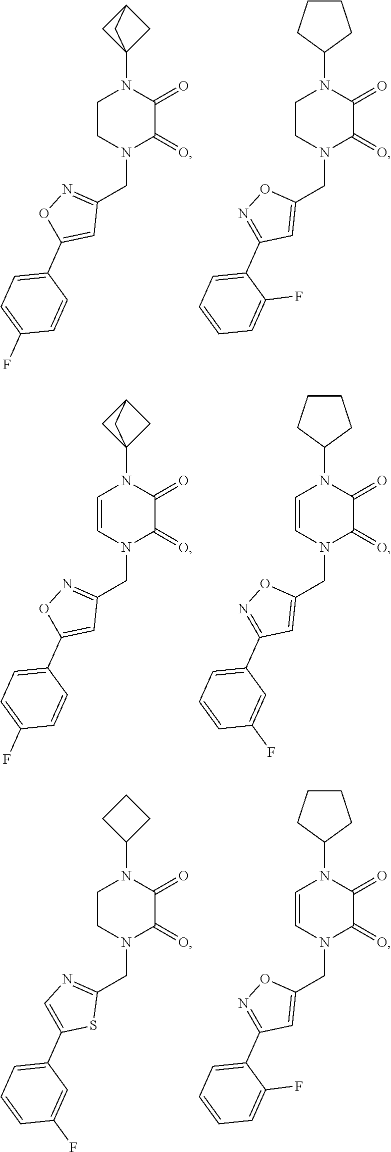 [SMILES: O=C1C(=O)N(C23CC(C2)C3)CCN1Cc1cc(-c2ccc(F)cc2)on1 ; O=C1C(=O)N(C2CCCC2)CCN1Cc1cc(-c2ccccc2F)no1 ; O=c1c(=O)n(C23CC(C2)C3)ccn1Cc1cc(-c2ccc(F)cc2)on1 ; O=c1c(=O)n(C2CCCC2)ccn1Cc1cc(-c2cccc(F)c2)no1 ; O=C1C(=O)N(C2CCC2)CCN1Cc1ncc(-c2cccc(F)c2)s1 ; O=c1c(=O)n(C2CCCC2)ccn1Cc1cc(-c2ccccc2F)no1] 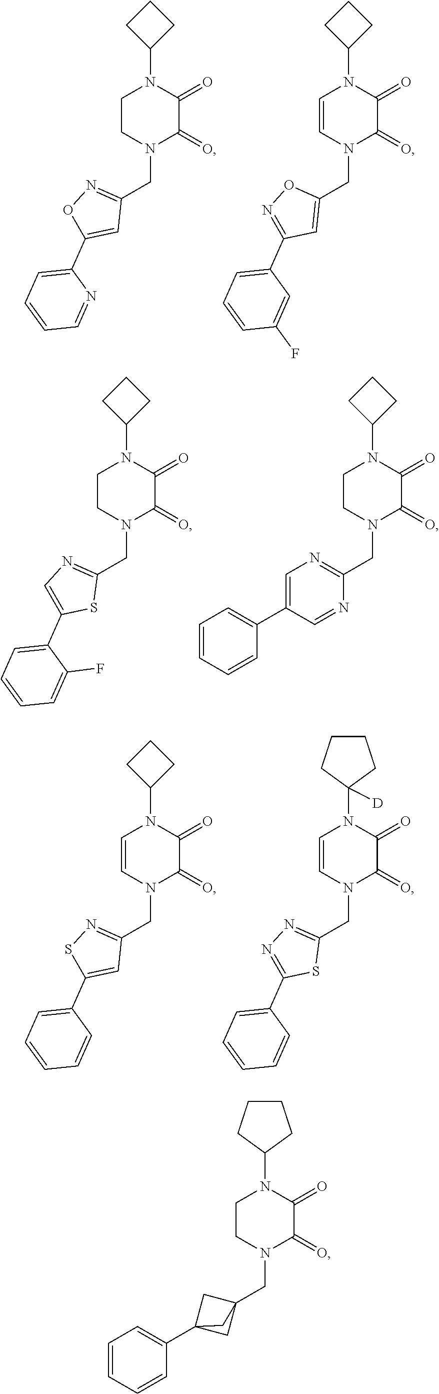 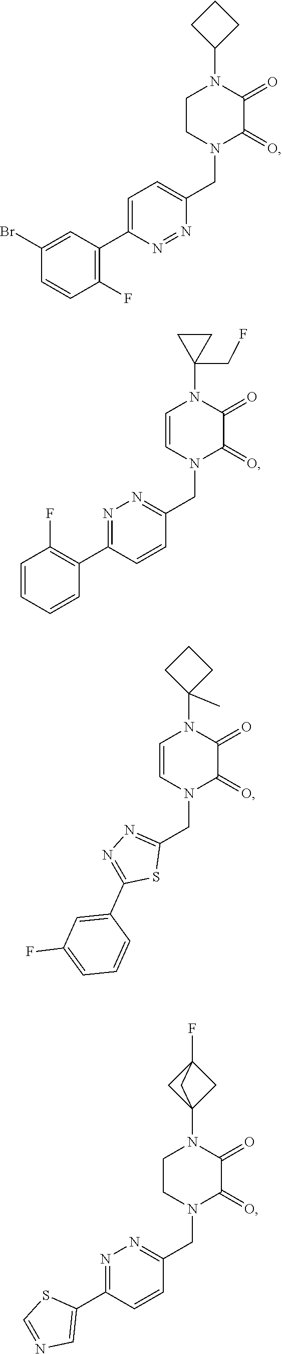 [SMILES: O=C1C(=O)N(C2CCC2)CCN1Cc1ccc(-c2cc(Br)ccc2F)nn1 ; O=c1c(=O)n(C2(CF)CC2)ccn1Cc1ccc(-c2ccccc2F)nn1 ; CC1(n2ccn(Cc3nnc(-c4cccc(F)c4)s3)c(=O)c2=O)CCC1 ; O=C1C(=O)N(C23CC(F)(C2)C3)CCN1Cc1ccc(-c2cncs2)nn1] 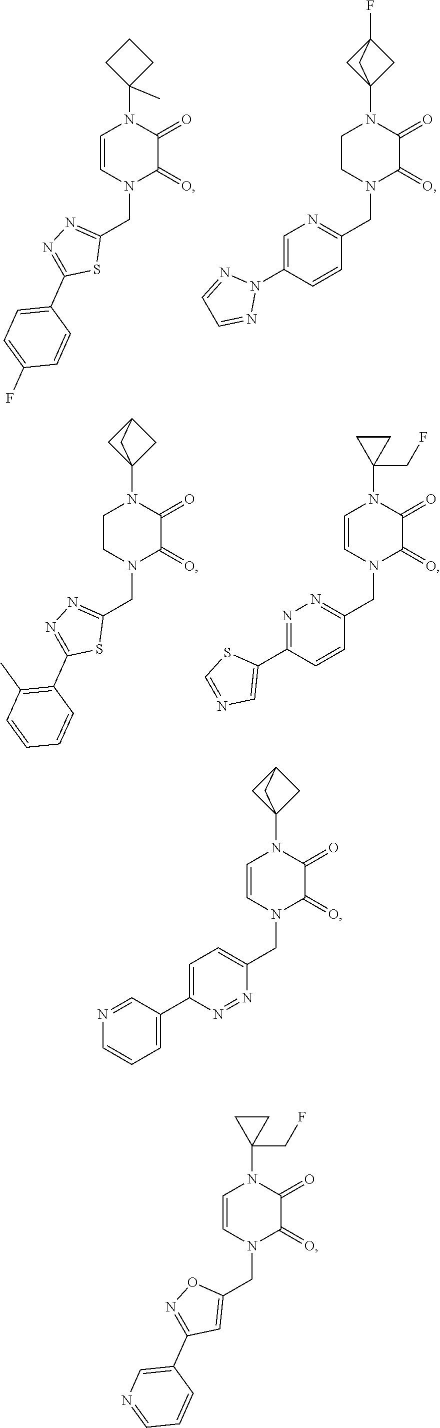 [SMILES: CC1(n2ccn(Cc3nnc(-c4ccc(F)cc4)s3)c(=O)c2=O)CCC1 ; O=C1C(=O)N(C23CC(F)(C2)C3)CCN1Cc1ccc(-n2nccn2)cn1 ; Cc1ccccc1-c1nnc(CN2CCN(C34CC(C3)C4)C(=O)C2=O)s1 ; O=c1c(=O)n(C2(CF)CC2)ccn1Cc1ccc(-c2cncs2)nn1 ; O=c1c(=O)n(C23CC(C2)C3)ccn1Cc1ccc(-c2cccnc2)nn1 ; O=c1c(=O)n(C2(CF)CC2)ccn1Cc1cc(-c2cccnc2)no1] 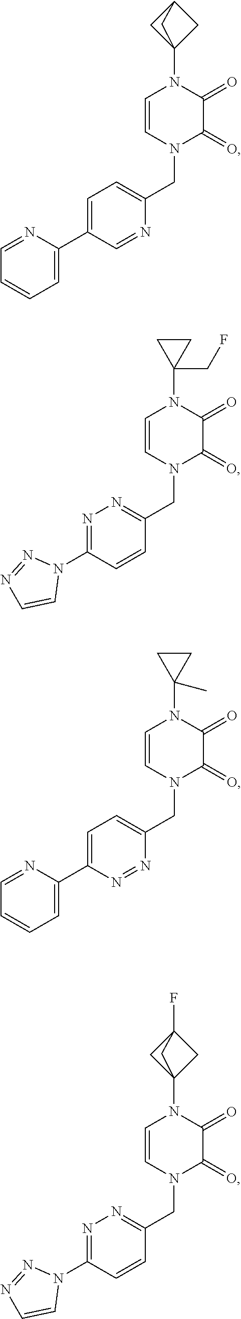 [SMILES: O=c1c(=O)n(C23CC(C2)C3)ccn1Cc1ccc(-c2ccccn2)cn1 ; O=c1c(=O)n(C2(CF)CC2)ccn1Cc1ccc(-n2ccnn2)nn1 ; CC1(n2ccn(Cc3ccc(-c4ccccn4)nn3)c(=O)c2=O)CC1 ; O=c1c(=O)n(C23CC(F)(C2)C3)ccn1Cc1ccc(-n2ccnn2)nn1] 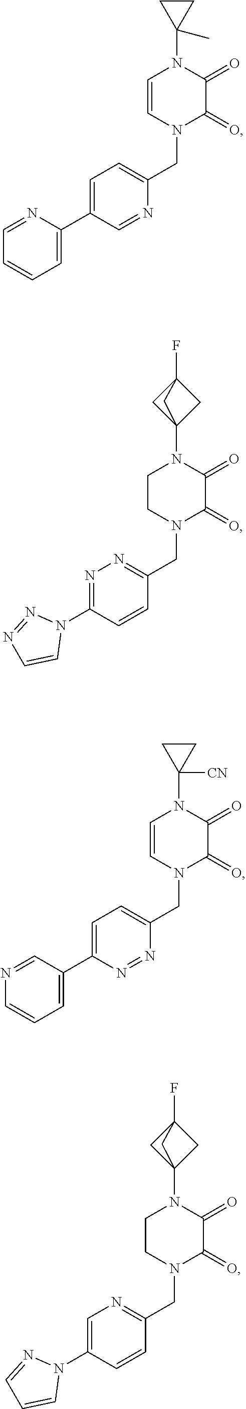 [SMILES: CC1(n2ccn(Cc3ccc(-c4ccccn4)cn3)c(=O)c2=O)CC1 ; O=C1C(=O)N(C23CC(F)(C2)C3)CCN1Cc1ccc(-n2ccnn2)nn1 ; N#CC1(n2ccn(Cc3ccc(-c4cccnc4)nn3)c(=O)c2=O)CC1 ; O=C1C(=O)N(C23CC(F)(C2)C3)CCN1Cc1ccc(-n2cccn2)cn1] 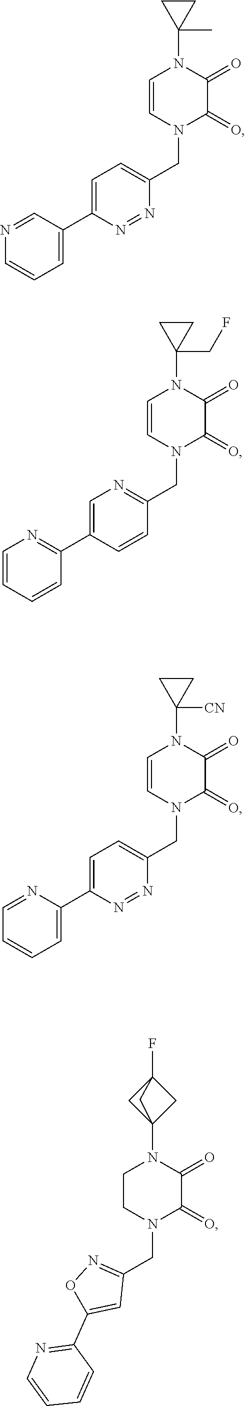 [SMILES: CC1(n2ccn(Cc3ccc(-c4cccnc4)nn3)c(=O)c2=O)CC1 ; O=c1c(=O)n(C2(CF)CC2)ccn1Cc1ccc(-c2ccccn2)cn1 ; N#CC1(n2ccn(Cc3ccc(-c4ccccn4)nn3)c(=O)c2=O)CC1 ; O=C1C(=O)N(C23CC(F)(C2)C3)CCN1Cc1cc(-c2ccccn2)on1] 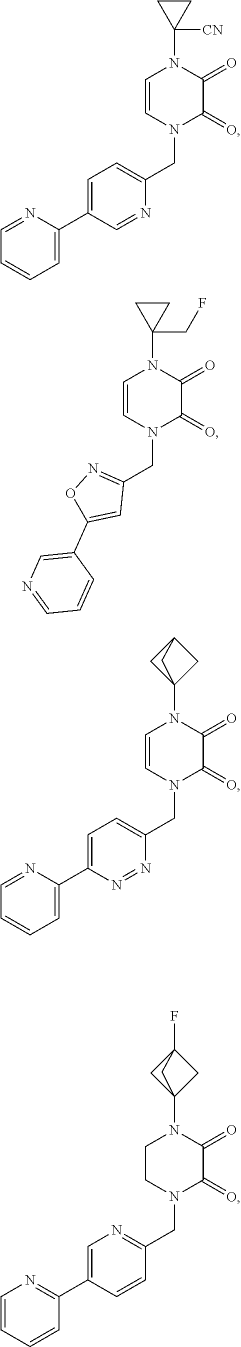 [SMILES: N#CC1(n2ccn(Cc3ccc(-c4ccccn4)cn3)c(=O)c2=O)CC1 ; O=c1c(=O)n(C2(CF)CC2)ccn1Cc1cc(-c2cccnc2)on1 ; O=c1c(=O)n(C23CC(C2)C3)ccn1Cc1ccc(-c2ccccn2)nn1 ; O=C1C(=O)N(C23CC(F)(C2)C3)CCN1Cc1ccc(-c2ccccn2)cn1] 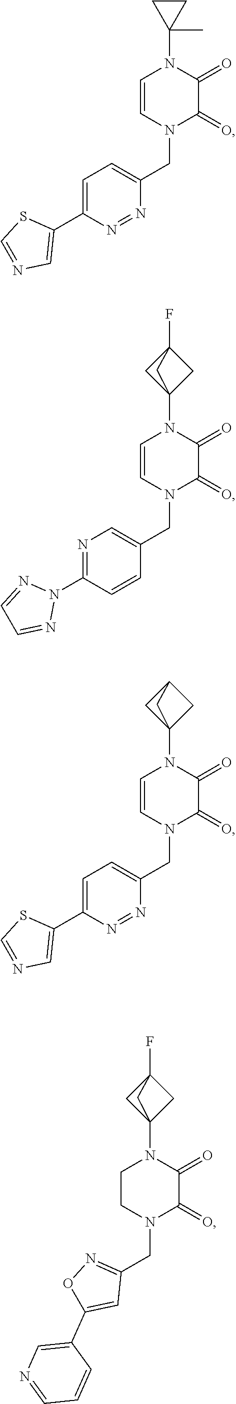 [SMILES: CC1(n2ccn(Cc3ccc(-c4cncs4)nn3)c(=O)c2=O)CC1 ; O=c1c(=O)n(C23CC(F)(C2)C3)ccn1Cc1ccc(-n2nccn2)nc1 ; O=c1c(=O)n(C23CC(C2)C3)ccn1Cc1ccc(-c2cncs2)nn1 ; O=C1C(=O)N(C23CC(F)(C2)C3)CCN1Cc1cc(-c2cccnc2)on1] 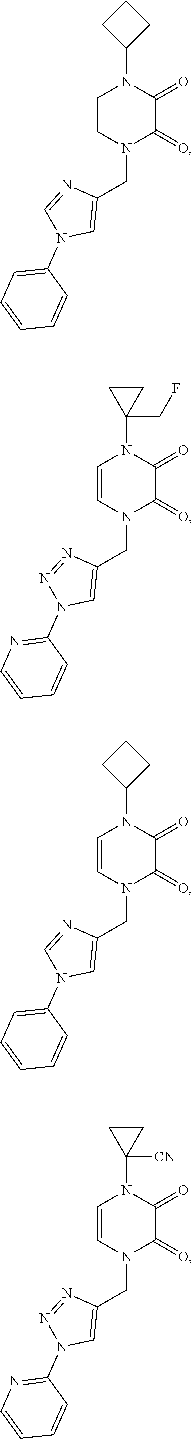 [SMILES: O=C1C(=O)N(C2CCC2)CCN1Cc1cn(-c2ccccc2)cn1 ; O=c1c(=O)n(C2(CF)CC2)ccn1Cc1cn(-c2ccccn2)nn1 ; O=c1c(=O)n(C2CCC2)ccn1Cc1cn(-c2ccccc2)cn1 ; N#CC1(n2ccn(Cc3cn(-c4ccccn4)nn3)c(=O)c2=O)CC1] 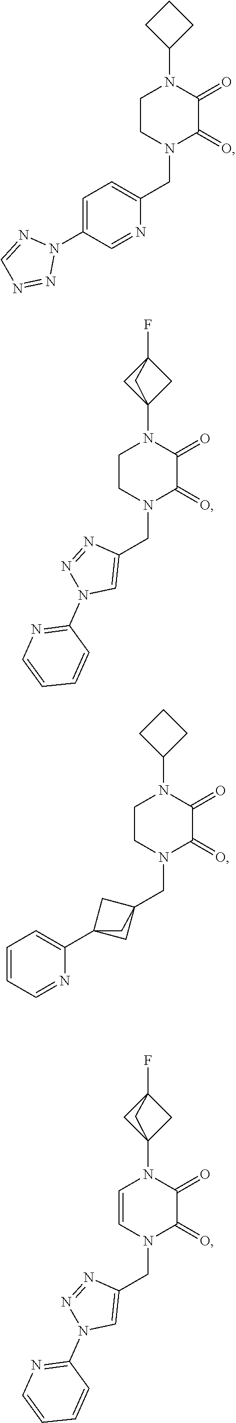 [SMILES: O=C1C(=O)N(C2CCC2)CCN1Cc1ccc(-n2ncnn2)cn1 ; O=C1C(=O)N(C23CC(F)(C2)C3)CCN1Cc1cn(-c2ccccn2)nn1 ; O=C1C(=O)N(C2CCC2)CCN1CC12CC(c3ccccn3)(C1)C2 ; O=c1c(=O)n(C23CC(F)(C2)C3)ccn1Cc1cn(-c2ccccn2)nn1] 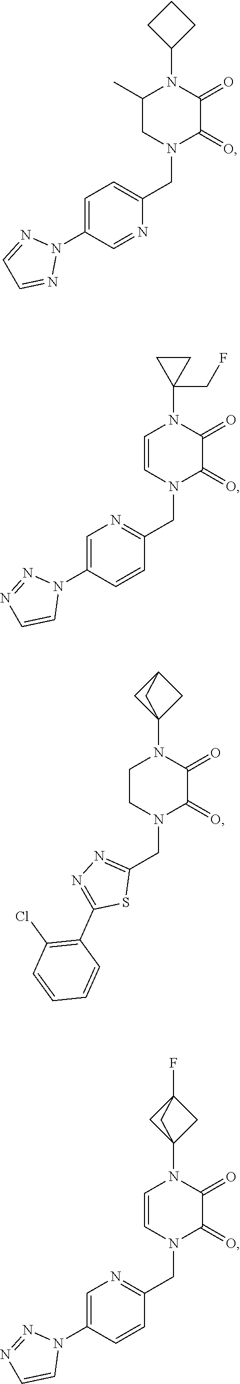 [SMILES: CC1CN(Cc2ccc(-n3nccn3)cn2)C(=O)C(=O)N1C1CCC1 ; O=c1c(=O)n(C2(CF)CC2)ccn1Cc1ccc(-n2ccnn2)cn1 ; O=C1C(=O)N(C23CC(C2)C3)CCN1Cc1nnc(-c2ccccc2Cl)s1 ; O=c1c(=O)n(C23CC(F)(C2)C3)ccn1Cc1ccc(-n2ccnn2)cn1] 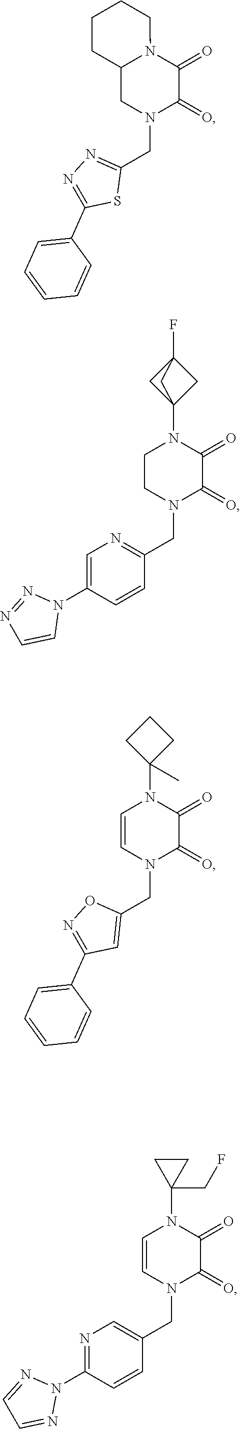 [SMILES: O=C1C(=O)N2CCCCC2CN1Cc1nnc(-c2ccccc2)s1 ; O=C1C(=O)N(C23CC(F)(C2)C3)CCN1Cc1ccc(-n2ccnn2)cn1 ; CC1(n2ccn(Cc3cc(-c4ccccc4)no3)c(=O)c2=O)CCC1 ; O=c1c(=O)n(C2(CF)CC2)ccn1Cc1ccc(-n2nccn2)nc1] 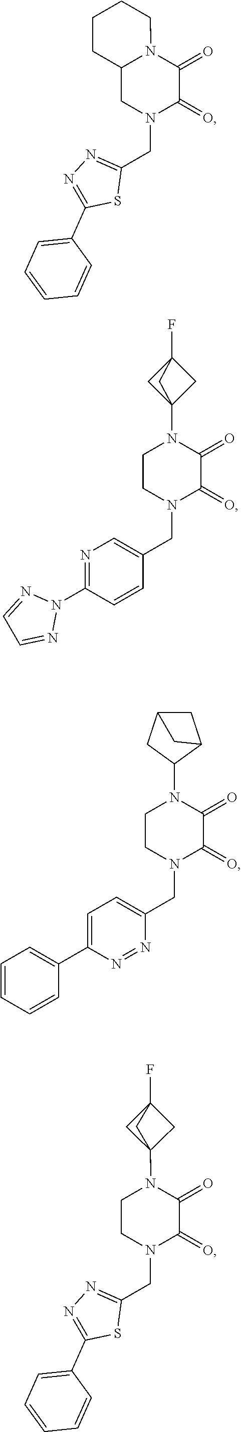 [SMILES: O=C1C(=O)N2CCCCC2CN1Cc1nnc(-c2ccccc2)s1 ; O=C1C(=O)N(C23CC(F)(C2)C3)CCN1Cc1ccc(-n2nccn2)nc1 ; O=C1C(=O)N(C2CC3CC2C3)CCN1Cc1ccc(-c2ccccc2)nn1 ; O=C1C(=O)N(C23CC(F)(C2)C3)CCN1Cc1nnc(-c2ccccc2)s1] 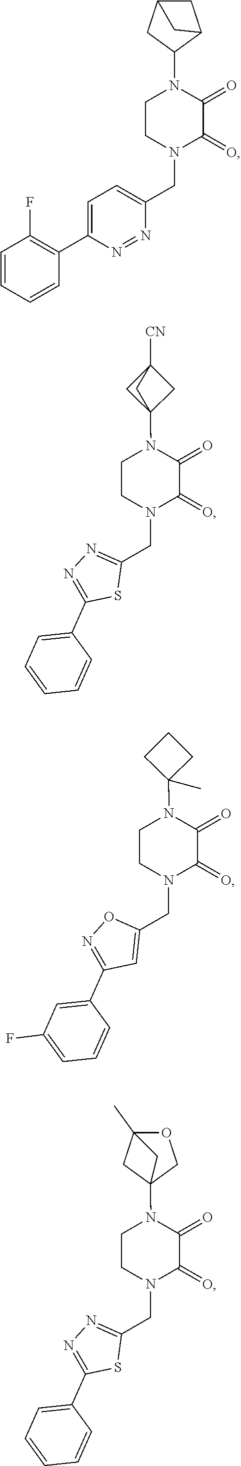 [SMILES: O=C1C(=O)N(C2CC3CC2C3)CCN1Cc1ccc(-c2ccccc2F)nn1 ; N#CC12CC(N3CCN(Cc4nnc(-c5ccccc5)s4)C(=O)C3=O)(C1)C2 ; CC1(N2CCN(Cc3cc(-c4cccc(F)c4)no3)C(=O)C2=O)CCC1 ; CC12CC(N3CCN(Cc4nnc(-c5ccccc5)s4)C(=O)C3=O)(CO1)C2] 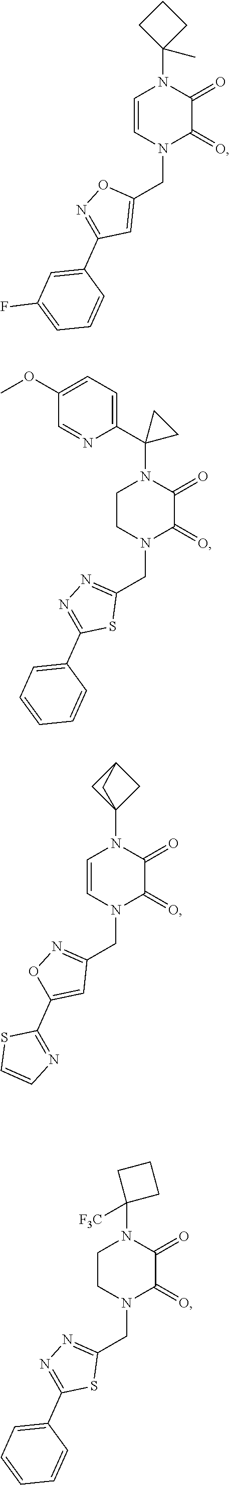 [SMILES: CC1(n2ccn(Cc3cc(-c4cccc(F)c4)no3)c(=O)c2=O)CCC1 ; COc1ccc(C2(N3CCN(Cc4nnc(-c5ccccc5)s4)C(=O)C3=O)CC2)nc1 ; O=c1c(=O)n(C23CC(C2)C3)ccn1Cc1cc(-c2nccs2)on1 ; O=C1C(=O)N(C2(C(F)(F)F)CCC2)CCN1Cc1nnc(-c2ccccc2)s1] 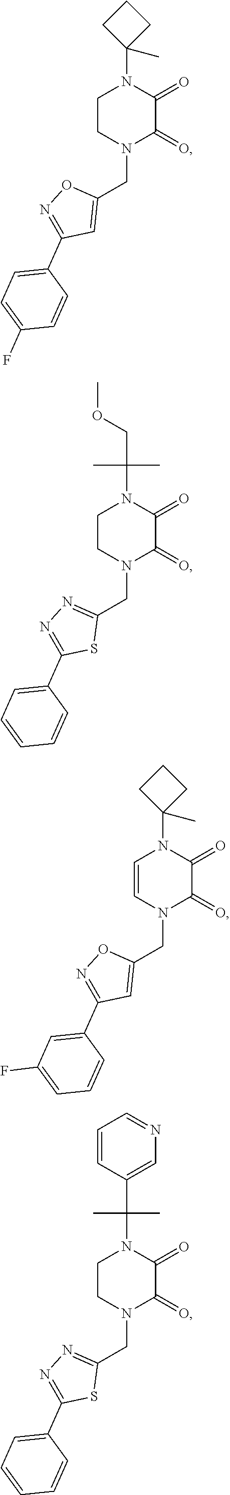 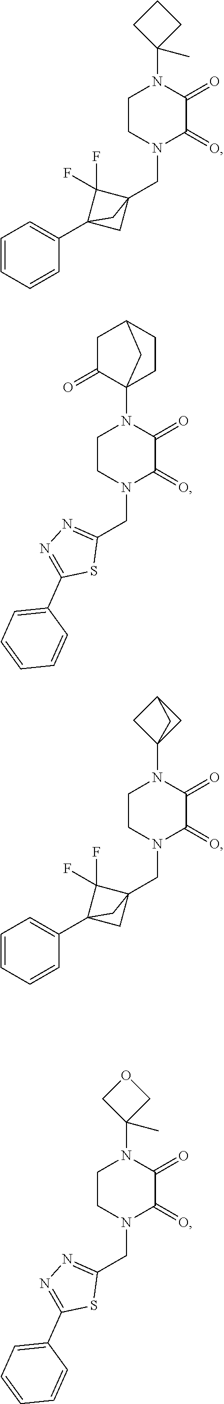 [SMILES: CC1(N2CCN(CC34CC(c5ccccc5)(C3)C4(F)F)C(=O)C2=O)CCC1 ; O=C1C(=O)N(C23CCC(CC2=O)C3)CCN1Cc1nnc(-c2ccccc2)s1 ; O=C1C(=O)N(C23CC(C2)C3)CCN1CC12CC(c3ccccc3)(C1)C2(F)F ; CC1(N2CCN(Cc3nnc(-c4ccccc4)s3)C(=O)C2=O)COC1] 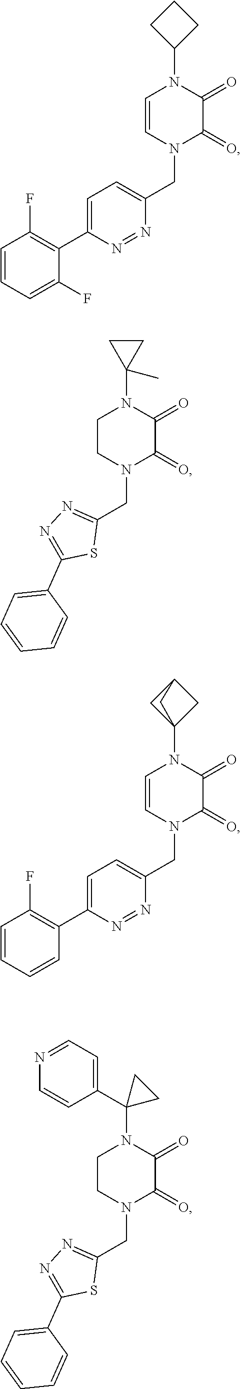 [SMILES: O=c1c(=O)n(C2CCC2)ccn1Cc1ccc(-c2c(F)cccc2F)nn1 ; CC1(N2CCN(Cc3nnc(-c4ccccc4)s3)C(=O)C2=O)CC1 ; O=c1c(=O)n(C23CC(C2)C3)ccn1Cc1ccc(-c2ccccc2F)nn1 ; O=C1C(=O)N(C2(c3ccncc3)CC2)CCN1Cc1nnc(-c2ccccc2)s1] 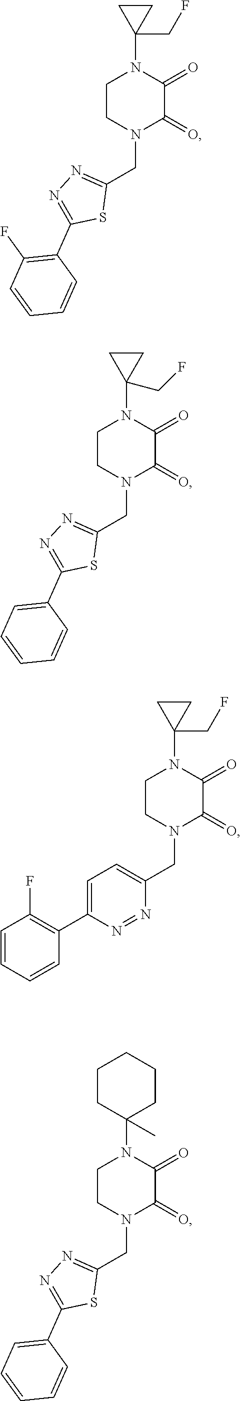 [SMILES: O=C1C(=O)N(C2(CF)CC2)CCN1Cc1nnc(-c2ccccc2F)s1 ; O=C1C(=O)N(C2(CF)CC2)CCN1Cc1nnc(-c2ccccc2)s1 ; O=C1C(=O)N(C2(CF)CC2)CCN1Cc1ccc(-c2ccccc2F)nn1 ; CC1(N2CCN(Cc3nnc(-c4ccccc4)s3)C(=O)C2=O)CCCCC1] 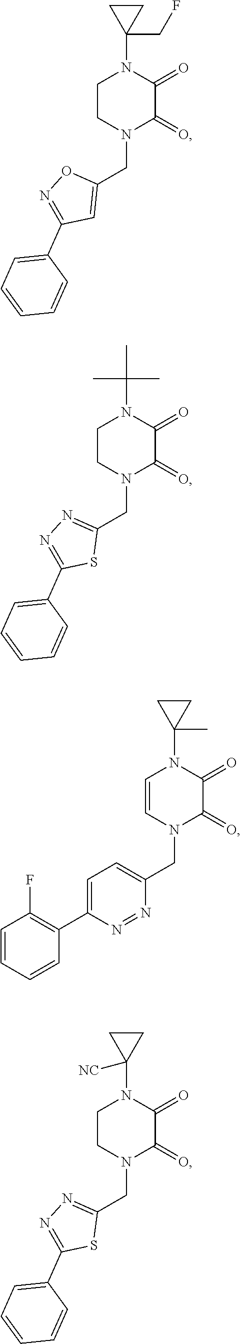 [SMILES: O=C1C(=O)N(C2(CF)CC2)CCN1Cc1cc(-c2ccccc2)no1 ; CC(C)(C)N1CCN(Cc2nnc(-c3ccccc3)s2)C(=O)C1=O ; CC1(n2ccn(Cc3ccc(-c4ccccc4F)nn3)c(=O)c2=O)CC1 ; N#CC1(N2CCN(Cc3nnc(-c4ccccc4)s3)C(=O)C2=O)CC1] 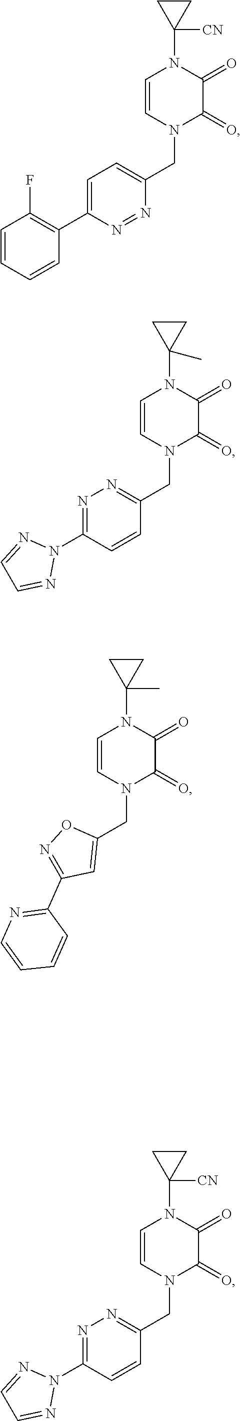 [SMILES: N#CC1(n2ccn(Cc3ccc(-c4ccccc4F)nn3)c(=O)c2=O)CC1 ; CC1(n2ccn(Cc3ccc(-n4nccn4)nn3)c(=O)c2=O)CC1 ; CC1(n2ccn(Cc3cc(-c4ccccn4)no3)c(=O)c2=O)CC1 ; N#CC1(n2ccn(Cc3ccc(-n4nccn4)nn3)c(=O)c2=O)CC1] 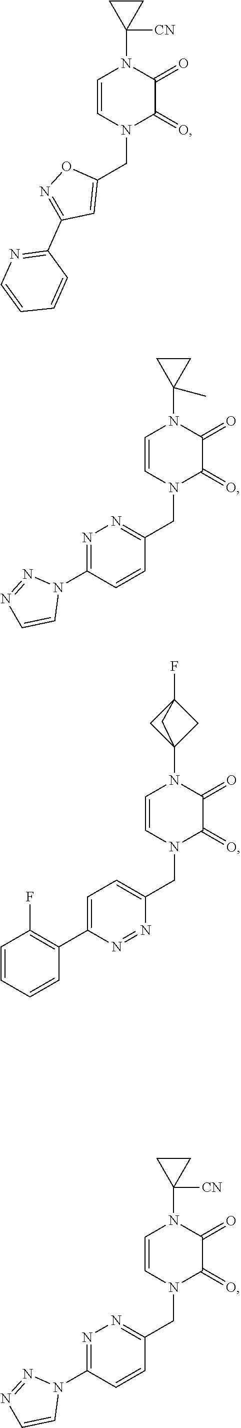 [SMILES: N#CC1(n2ccn(Cc3cc(-c4ccccn4)no3)c(=O)c2=O)CC1 ; CC1(n2ccn(Cc3ccc(-n4ccnn4)nn3)c(=O)c2=O)CC1 ; O=c1c(=O)n(C23CC(F)(C2)C3)ccn1Cc1ccc(-c2ccccc2F)nn1 ; N#CC1(n2ccn(Cc3ccc(-n4ccnn4)nn3)c(=O)c2=O)CC1] 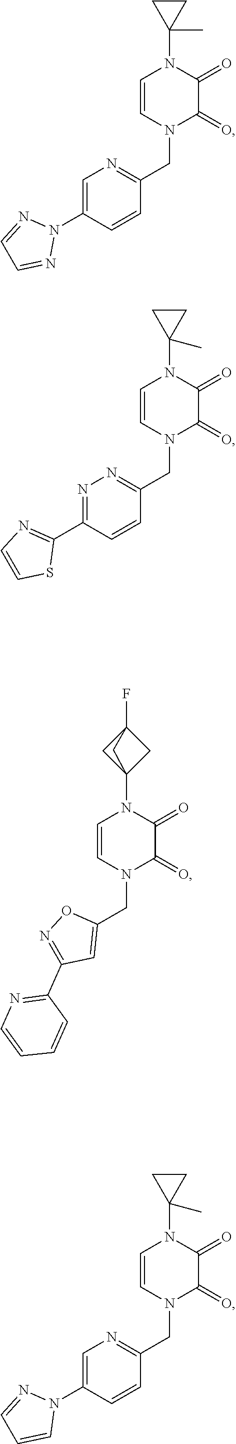 [SMILES: CC1(n2ccn(Cc3ccc(-n4nccn4)cn3)c(=O)c2=O)CC1 ; CC1(n2ccn(Cc3ccc(-c4nccs4)nn3)c(=O)c2=O)CC1 ; O=c1c(=O)n(C23CC(F)(C2)C3)ccn1Cc1cc(-c2ccccn2)no1 ; CC1(n2ccn(Cc3ccc(-n4cccn4)cn3)c(=O)c2=O)CC1] 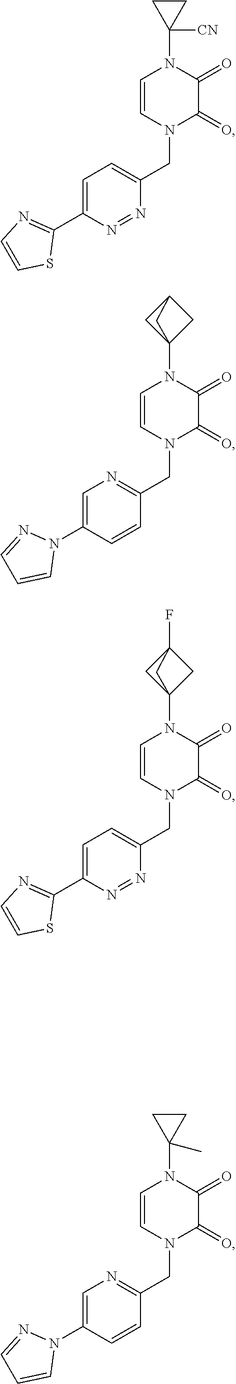 [SMILES: N#CC1(n2ccn(Cc3ccc(-c4nccs4)nn3)c(=O)c2=O)CC1 ; O=c1c(=O)n(C23CC(C2)C3)ccn1Cc1ccc(-n2cccn2)cn1 ; O=c1c(=O)n(C23CC(F)(C2)C3)ccn1Cc1ccc(-c2nccs2)nn1 ; CC1(n2ccn(Cc3ccc(-n4cccn4)cn3)c(=O)c2=O)CC1] 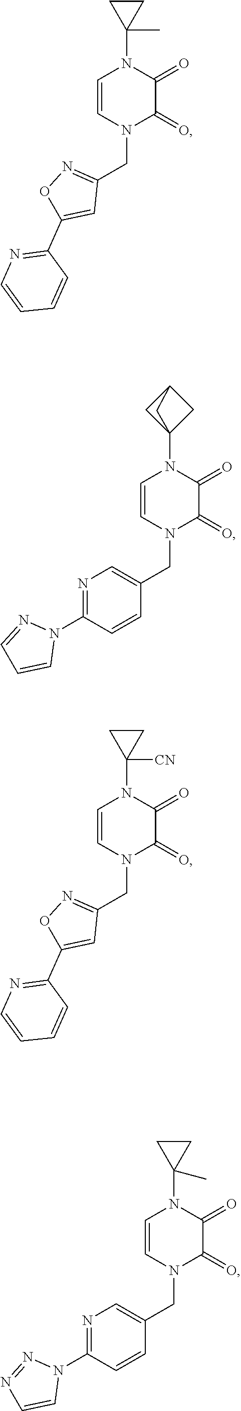 [SMILES: CC1(n2ccn(Cc3cc(-c4ccccn4)on3)c(=O)c2=O)CC1 ; O=c1c(=O)n(C23CC(C2)C3)ccn1Cc1ccc(-n2cccn2)nc1 ; N#CC1(n2ccn(Cc3cc(-c4ccccn4)on3)c(=O)c2=O)CC1 ; CC1(n2ccn(Cc3ccc(-n4ccnn4)nc3)c(=O)c2=O)CC1] 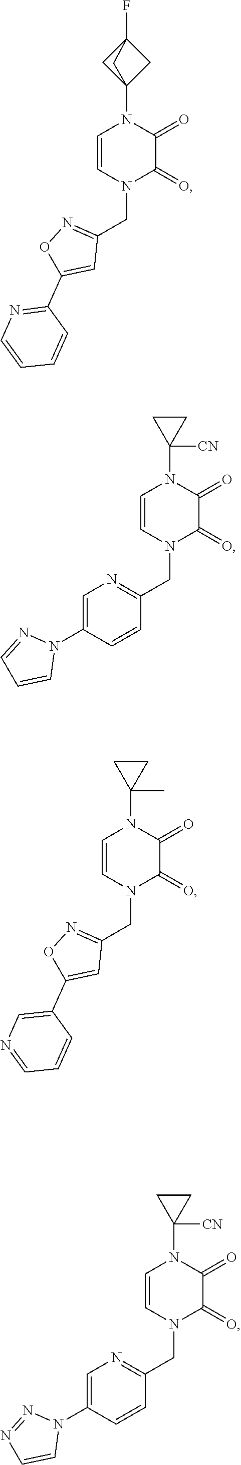 [SMILES: O=c1c(=O)n(C23CC(F)(C2)C3)ccn1Cc1cc(-c2ccccn2)on1 ; N#CC1(n2ccn(Cc3ccc(-n4cccn4)cn3)c(=O)c2=O)CC1 ; CC1(n2ccn(Cc3cc(-c4cccnc4)on3)c(=O)c2=O)CC1 ; N#CC1(n2ccn(Cc3ccc(-n4ccnn4)cn3)c(=O)c2=O)CC1] 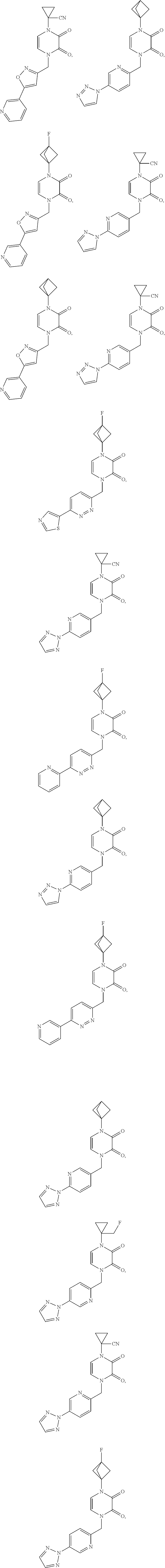 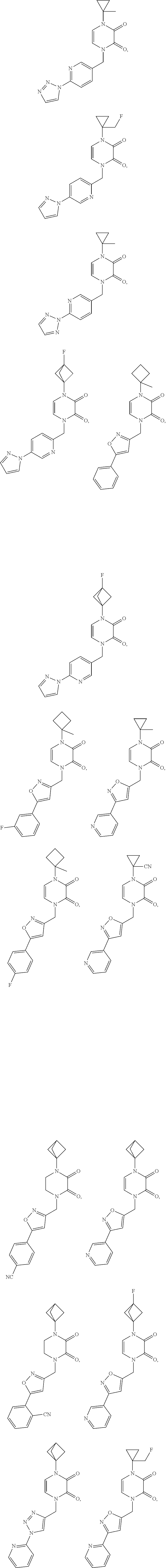 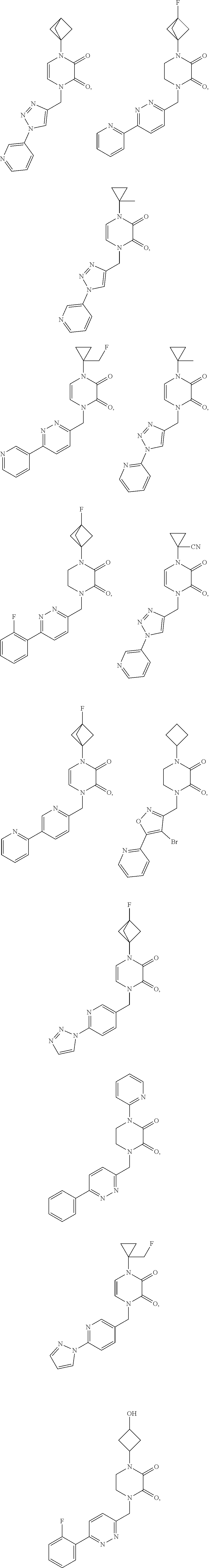 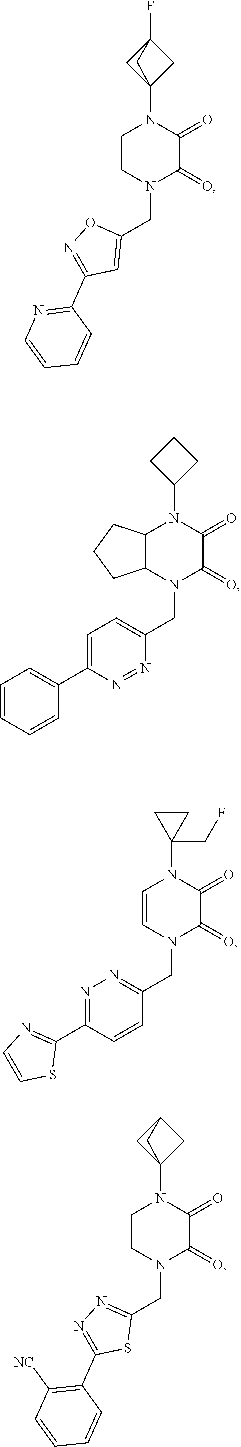 [SMILES: O=C1C(=O)N(C23CC(F)(C2)C3)CCN1Cc1cc(-c2ccccn2)no1 ; O=C1C(=O)N(C2CCC2)C2CCCC2N1Cc1ccc(-c2ccccc2)nn1 ; O=c1c(=O)n(C2(CF)CC2)ccn1Cc1ccc(-c2nccs2)nn1 ; N#Cc1ccccc1-c1nnc(CN2CCN(C34CC(C3)C4)C(=O)C2=O)s1] 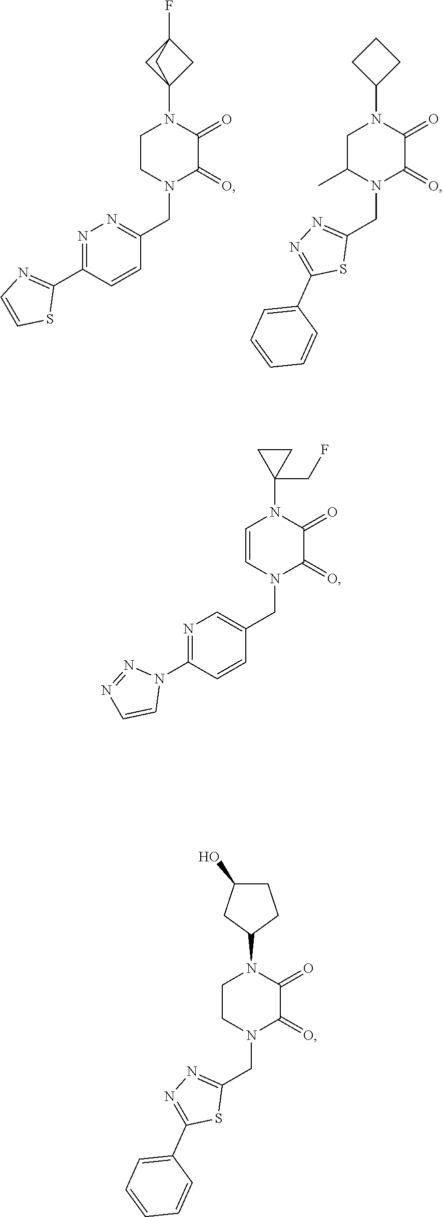 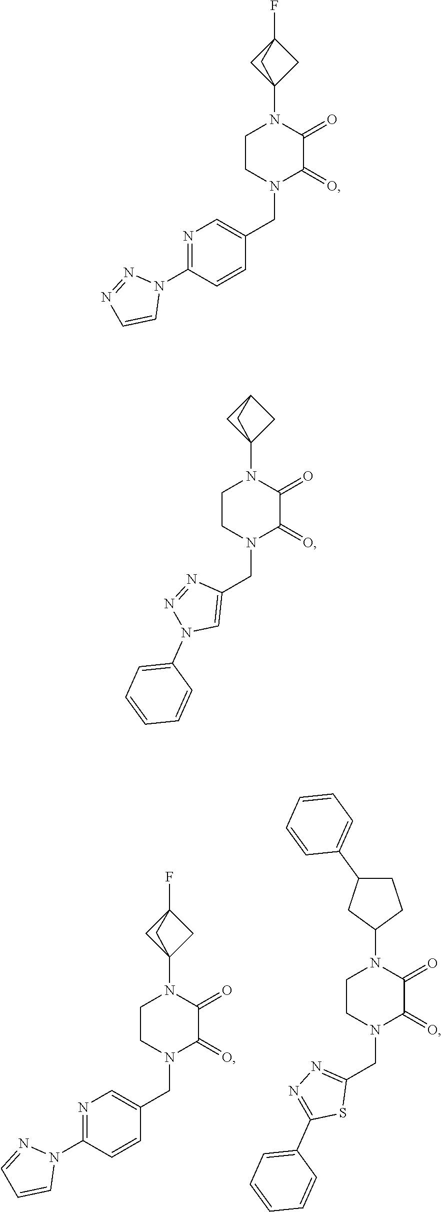 [SMILES: O=C1C(=O)N(C23CC(F)(C2)C3)CCN1Cc1ccc(-n2ccnn2)nc1 ; O=C1C(=O)N(C23CC(C2)C3)CCN1Cc1cn(-c2ccccc2)nn1 ; O=C1C(=O)N(C23CC(F)(C2)C3)CCN1Cc1ccc(-n2cccn2)nc1 ; O=C1C(=O)N(C2CCC(c3ccccc3)C2)CCN1Cc1nnc(-c2ccccc2)s1] 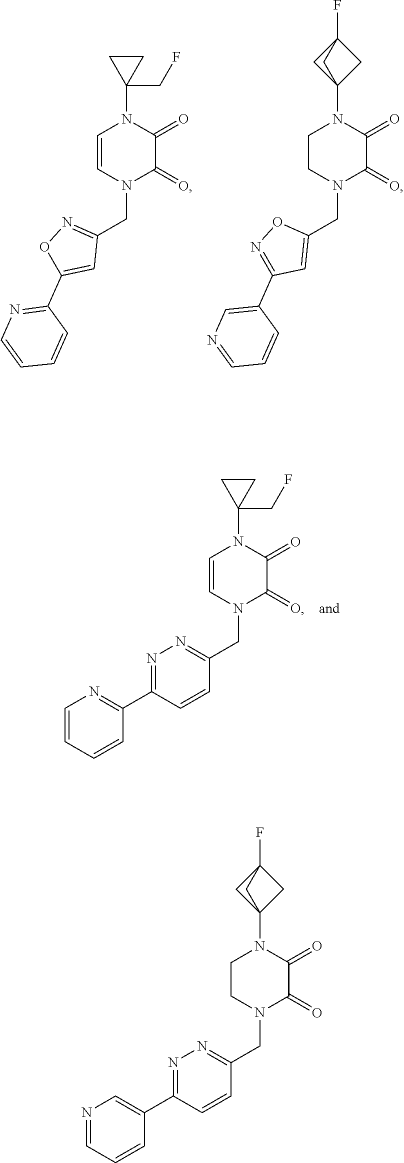 [SMILES: O=c1c(=O)n(C2(CF)CC2)ccn1Cc1cc(-c2ccccn2)on1 ; O=C1C(=O)N(C23CC(F)(C2)C3)CCN1Cc1cc(-c2cccnc2)no1 ; O=c1c(=O)n(C2(CF)CC2)ccn1Cc1ccc(-c2ccccn2)nn1 ; O=C1C(=O)N(C23CC(F)(C2)C3)CCN1Cc1ccc(-c2cccnc2)nn1]
or a pharmaceutically acceptable salt thereof.

28. A compound, or a pharmaceutically acceptable salt thereof, wherein the compound is:

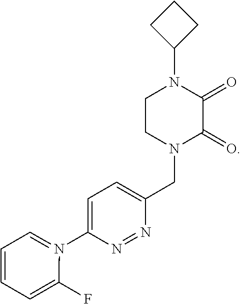

29. A compound, wherein the compound is:

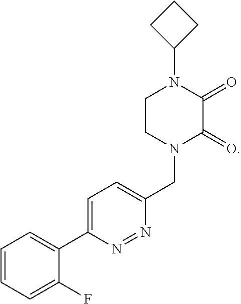 [SMILES: O=C1C(=O)N(C2CCC2)CCN1Cc1ccc(-c2ccccc2F)nn1]

30. A compound, or pharmaceutically acceptable salt thereof, wherein the compound is:

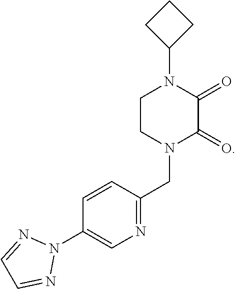 [SMILES: O=C1C(=O)N(C2CCC2)CCN1Cc1ccc(-n2nccn2)cn1]

31. A compound, wherein the compound is:

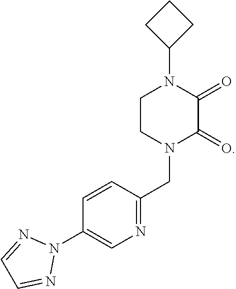 [SMILES: O=C1C(=O)N(C2CCC2)CCN1Cc1ccc(-n2nccn2)cn1]

32. A method of treating cancer in a patient comprising administering to a patient in need thereof a compound, or pharmaceutically acceptable salt thereof, of claim 1.

33. (canceled)

34. A pharmaceutical composition comprising a compound of claim 1, or a pharmaceutically acceptable salt thereof, and a pharmaceutically acceptable carrier.

35. (canceled)
